(19) United States
(12) Patent Application Publication
(10) Pub. No.: US 2023/0192896 A1
(43) Pub. Date: Jun. 22, 2023

(54) BISPECIFIC ANTIBODIES BINDING TO COAGULATION FACTOR IX AND COAGULATION FACTOR X

(71) Applicant: Bioverativ Therapeutics Inc., Waltham, MA (US)

(72) Inventors: Robert T. PETERS, Needham, MA (US); Nina LEKSA, Waltham, MA (US); Bradley R. PEARSE, Watertown, MA (US); John KULMAN, Belmont, MA (US); Maria ALEMAN, Waltham, MA (US); Allison GOODMAN, Waltham, MA (US)

(21) Appl. No.: 16/462,878

(22) PCT Filed: Nov. 22, 2017
(86) PCT No.: PCT/US2017/063135

§ 371 Date: May 21, 2019

Related U.S. Application Data
(60) Provisional application No. 62/587,284, filed on Nov. 16, 2017.
(60) Provisional application No. 62/529,805, filed on Jul. 7, 2017.
(60) Provisional application No. 62/452,809, filed on Jan. 31, 2017.
(60) Provisional application No. 62/425,921, filed on Nov. 23, 2016.

(51) Int. Cl. C07K 16/46; A61P 7/04

(52) U.S. Cl. CPC C07K 16/468; A61P 7/04; C07K 2317/565; C07K 2317/31; C07K 2317/34

(57) ABSTRACT

The present disclosure provides antibodies that selectively binds to specific forms of clotting factors, in particular, antibodies that specifically binds to activated factor IX (FIXa) wherein the anti-FIXa antibody or an antigen binding portion thereof preferentially binds to FIXa in the presence of FIXa and factor IX zymogen (FIXz), and antibodies that specifically bind to factor X zymogen (FXz) wherein the anti-FXz antibody or antigen binding portion thereof preferentially binds to FXz in the presence of FXz and activated factor X (FXa). Also provided are bispecific molecules (e.g., antibodies) comprising, e.g., an anti-FIXa antibody or antigen binding portion thereof and/or an anti-FXz antibody or antigen binding portion thereof. The disclosure also provides compositions encoding the disclosed antibodies and bispecific molecules, vectors, cells, pharmaceutical and diagnostic compositions, kits, methods of manufacture, methods of use, and immunoconjugates.

### REFERENCE TO EARLIER FILED APPLICATIONS

The present application claims benefit to U.S. Provisional Application No. 62/425,921, filed Nov. 23, 2016, U.S. Provisional Application No. 62/452,809, filed Jan. 31, 2017, U.S. Provisional Application No. 62/529,805, filed Jul. 7, 2017, and U.S. Provisional Application No. 62/587,284, filed Nov. 16, 2017, which are incorporated herein by reference in their entireties.

### REFERENCE TO SEQUENCE LISTING SUBMITTED ELECTRONICALLY VIA EFS-WEB

The content of the electronically submitted sequence listing (Name: 4159.485PC04_Sequence_listing_ST25.txt; Size: 1,053,370 bytes; and Date of Creation: Nov. 21, 2017) filed with the application is incorporated herein by reference in its entirety.

### BACKGROUND

### Field

This application pertains to, among other things, antibodies that preferentially bind to activated coagulation factor IX or coagulation factor X zymogen, as well as bispecific molecules comprising both specificities that mimic the activated factor VIII cofactor.

### Background

Hemophilia A is a severe X-chromosome-linked recessive disorder caused by mutations in the factor VIII (FVIII) gene. FVIII is involved in the intrinsic pathway of blood coagulation, and FVIII deficiency leads to blood either coagulating poorly, or barely at all. FVIII deficiency, alternatively known as hemophilia A, is one of the most common hemorrhagic disorders, and affects one in about 10,000 males (Stonebraker et al. (2012) Haemophilia 18(3):e91-4). Hemophilia A has three grades of severity defined by factor FVIII plasma levels of 1% or less (“severe”), 2 to 5% (“moderate”), and 6 to 30% (“mild”) (White et al. (2001) Thromb. Haemost. 85:560). In severe forms of the disorder, the first bleeds typically appear at 5 to 6 months of age, whereas the first bleeds are delayed until about 1 to 2 years of age in the moderate form. A bleed can appear spontaneously, or following minimum trauma. Approximately half of all patients with hemophilia A are classified as having the severe form of the disease. These patients experience severe bleeding starting in early childhood, and frequent episodes of spontaneous or excessive bleeding later in life. Bleeding commonly occurs into joints and muscles, and without appropriate treatment, recurrent bleeding can lead to irreversible hemoarthropathy (Manco-Johnson et al. (2007) N. Engl. J. Med. 357(6):535-44).

An important goal of hemophilia A treatment is maintenance of FVIII plasma levels ≥1%, which reduces bleeding risk. To achieve this, intravenous recombinant or plasma-derived FVIII is administered frequently as prophylactic therapy. However, this current standard of treatment of hemophilia A is difficult, has several drawbacks, and incurs a considerable physical and mental burden on patients and their families.

The most common hindrance in FVIII treatment is the production of alloantibodies against FVIII, which act as FVIII inhibitors. As many as 30% of severely affected patients develop such alloantibodies, and once development has occurred, the effective use of FVIII for treating on-going bleeds is restricted (Kempton & White (2009) Blood 113(1):11-7). In such cases, alternative bypassing agents are used to control bleeding. However, these agents typically have shorter half-lives and are not always effective. Furthermore, frequent administration of FVIII is required due to its short plasma half-life (an average of about 12 hours in adults, and even shorter in children). Such a regimen can be difficult, particularly in young children. Since available treatments are associated with complications and side effects, there is no single treatment that optimally and effectively treats hemophilia. Thus, there remains an unmet need for new and effective treatments that resolve the drawbacks of treating hemophilia A with FVIII.

### BRIEF SUMMARY

The present disclosure provides an isolated antibody, or an antigen binding portion thereof, that specifically binds to activated factor IX (FIXa) (“anti-FIXa antibody or antigen binding portion thereof”), wherein the anti-FIXa antibody or antigen binding portion thereof preferentially binds to FIXa in the presence of FIXa and factor IX zymogen (FIXz). In some aspects, the anti-FIXa antibody, or antigen binding portion thereof binds to FIXa with a binding affinity higher than a binding affinity of the anti-FIXa antibody or antigen binding portion thereof to FIXz. The disclosure also provides an isolated anti-FIXa antibody, or antigen binding portion thereof, which binds to FIXa with a binding affinity higher than a binding affinity of the anti-FIXa antibody or antigen binding portion thereof to FIXz. In some aspects, the anti-FIXa antibody, or antigen binding portion thereof binds to FIXa with a K_{D} of about 100 nM or less, about 90 nM or less, about 80 nM or less, about 70 nM or less, about 60 nM or less, about 50 nM or less, about 40 nM or less, about 30 nM or less, about 20 nM or less, about 10 nM or less, about 8 nM or less, about 6 nM or less, about 4 nM or less, about 2 nM or less, about 1 nM or less as determined by a Bio-Layer Interferometry (BLI) assay. In some aspects, the FIXa is free FIXa, FIXa in a tenase complex, or FIXa covalently linked to EGR-CMK (FIXa-SM). In some aspects, the FIXz comprises non-activatable Factor IX (FIXn). In some aspects, the anti-FIXa antibody, or antigen binding portion thereof cross-competes with a reference antibody selected from the group consisting of the antibodies in FIG. 3A, FIG. 3B, and FIG. 3C. In some aspects, the anti-FIXa antibody, or antigen binding portion thereof binds to the same epitope as a reference antibody selected from the group consisting of the antibodies in FIG. 3A, FIG. 3B, and FIG. 3C. In some aspects, such reference antibody is selected from BIIB-9-484, BIIB-9-440, BIIB-9-882, BIIB-9-460, BIIB-9-433, and any combination thereof. In some aspects, the anti-FIXa antibody, or antigen binding portion thereof preferentially binds to FIXa-SM compared to free FIXa or FIXz and/or binds to FIXa-SM with a binding affinity higher than a binding affinity of the anti-FIXa antibody or antigen binding portion thereof to free FIXa or FIXz. In some aspects, the anti-FIXa antibody, or antigen binding portion thereof cross-competes with a reference antibody selected from the group consisting of the antibodies in FIG. 3A. In some aspects, the anti-FIXa antibody, or antigen binding portion thereof binds to the same epitope as a reference antibody selected from the group consisting of the antibodies in FIG. 3A. In some aspects, such reference antibody is selected from BIIB-9-484, BIIB-9-440, BIIB-9-460, and any combination thereof. In some aspects, the anti-FIXa antibody, or antigen binding portion thereof preferentially binds to free FIXa compared to FIXa-SM or FIXz and/or binds to free FIXa with a binding affinity higher than a binding affinity of the anti-FIXa antibody or antigen binding portion thereof to FIXa-SM or FIXz. In some aspects, the anti-FIXa antibody, or antigen binding portion thereof, cross-competes with a reference antibody selected from the group consisting of the antibodies in FIG. 3B. In some aspects, the anti-FIXa antibody, or antigen binding portion thereof binds to the same epitope as a reference antibody selected from the group consisting of the antibodies in FIG. 3B. In some aspects, the anti-FIXa antibody, or antigen binding portion thereof preferentially binds to free FIXa or FIXa-SM compared to FIXz and/or binds to free FIXa or FIXa-SM with a binding affinity higher than a binding affinity of the anti-FIXa antibody or antigen binding portion thereof to FIXz. In some aspects, the anti-FIXa antibody, or antigen binding portion thereof cross-competes with a reference antibody selected from the group consisting of the antibodies in FIG. 3C. In some aspects, the anti-FIXa antibody, or antigen binding portion thereof binds to the same epitope as a reference antibody selected from the group consisting of the antibodies in FIG. 3C. In some aspects, such reference antibody is selected from BIIB-9-882, BIIB-9-433, and the combination thereof. In some aspects, the anti-FIXa antibody, or antigen binding portion thereof comprises CDR1, CDR2, and CDR3, wherein the CDR3 comprises a VH CDR3 selected from the group consisting of VH CDR3s in FIG. 3A, FIG. 3B, and FIG. 3C or the VH CDR3 with one or two mutations. In some aspects, the anti-FIXa antibody, or antigen binding portion thereof comprises CDR1, CDR2, and CDR3, wherein the CDR3 comprises ARDX₁X₂X₃X₄X₅X₆YYX₇MDV (SEQ ID NO:753), wherein X₁ is V or G, X₂ is G or V, X₃ is G or R, X₄ is Y or V, X₅ is A or S, X₆ is G or D, X₇ is G or none. In some aspects, the CDR3 comprises ARDVGGYAGYYGMDV (SEQ ID NO: 905, BIIB-9-484, 1335, 1336), ARDISTDGESSLYYYMDV (SEQ ID NO: 901, BIIB-9-460), ARGPTDSSGYLDMDV (SEQ ID NO: 1186, BIIB-9-882), or ARDGPRVSDYY MDV (SEQ ID NO: 912, BIIB-9-619). In some aspects, the anti-FIXa antibody, or antigen binding portion thereof comprises CDR1, CDR2, and CDR3, wherein the CDR1 comprises a VH CDR1 selected from the group consisting of VH CDR1s in FIG. 3A, FIG. 3B, and FIG. 3C or the VH CDR1 with one or two mutations. In some aspects, the anti-FIXa antibody, or antigen binding portion thereof comprises VH CDR1, CDR2, and CDR3, wherein the CDR2 comprises a VH CDR2 selected from the group consisting of VH CDR2s in FIG. 3A, FIG. 3B, and FIG. 3C or the VH CDR2 with one or two mutations. In some aspects, the anti-FIXa antibody, or antigen binding portion thereof comprises VL CDR1, CDR2, and CDR3, wherein the CDR1 comprises a VL CDR1 selected from the group consisting of VL CDR1s in FIG. 3A, FIG. 3B, and FIG. 3C or the VL CDR1 with one or two mutations. In some aspects, the anti-FIXa antibody, or antigen binding portion thereof comprises VL CDR1, CDR2, and CDR3, wherein the CDR2 comprises a VL CDR2 selected from the group consisting of VL CDR2s in FIG. 3A, FIG. 3B, and FIG. 3C or the VL CDR2 with one or two mutations. In some aspects, the anti-FIXa antibody, or antigen binding portion thereof comprises VL CDR1, CDR2, and CDR3, wherein the CDR3 comprises a VL CDR3 selected from the group consisting of VL CDR3s in FIG. 3A, FIG. 3B, and FIG. 3C or the VL CDR3 with one or two mutations.

The present disclosure also provides an isolated anti-FIXa antibody, or antigen binding portion thereof, which specifically binds to FIXa, comprising VH CDR1, CDR2, and CDR3 and VL CDR1, CDR2, and CDR3, wherein the VH CDR1, CDR2, and CDR3 and the VL CDR1, CDR2, and CDR3 comprise VH CDR1, VH CDR2, and VH CDR3 and VL CDR1, CDR2, and CDR3 of FIG. 3A, FIG. 3B, and FIG. 3C, respectively. In some aspects, the anti-FIXa antibody or antigen binding portion thereof comprises VH CDR1, CDR2, and CDR3 sequences comprising SEQ ID NOs: 815, 860, and 905, respectively, and/or VL CDR1, CDR2, and CDR3 sequences comprising SEQ ID NOs: 950, 995, and 1040, respectively (BIIB-9-484). In some aspects, the anti-FIXa antibody, or antigen binding portion thereof comprises VH CDR1, CDR2, and CDR3 sequences comprising SEQ ID NO: 809, SEQ ID NO: 854, and SEQ ID NO: 899, respectively, and/or VL CDR1, VL CDR2, and VL CDR3 sequences comprising SEQ ID NO: 944, SEQ ID NO: 989, and SEQ ID NO: 1034, respectively (BIIB-9-440). In some aspects, the anti-FIXa antibody, or antigen binding portion thereof comprises VH CDR1, CDR2, and CDR3 sequences comprising SEQ ID NO: 1102, SEQ ID NO: 1144, and SEQ ID NO: 1186, respectively, and/or VL CDR1, VL CDR2, and VL CDR3 sequences comprising SEQ ID NO: 1228, SEQ ID NO: 1270, and SEQ ID NO: 1312, respectively (BIIB-9-882). In some aspects, the anti-FIXa antibody, or antigen binding portion thereof comprises VH CDR1, CDR2, and CDR3 sequences comprising SEQ ID NO: 811, SEQ ID NO: 856, and SEQ ID NO: 901, respectively, and/or VL CDR1, VL CDR2, and VL CDR3 sequences comprising SEQ ID NO: 946, SEQ ID NO: 991, and SEQ ID NO: 1036, respectively (BIIB-9-460). In some aspects, the anti-FIXa antibody, or antigen binding portion thereof comprises VH CDR1, CDR2, and CDR3 sequences comprising SEQ ID NO: 1108, SEQ ID NO: 1150, and SEQ ID NO: 1192, respectively, and/or VL CDR1, VL CDR2, and VL CDR3 sequences comprising SEQ ID NO: 1234, SEQ ID NO: 1276, and SEQ ID NO: 1318, respectively (BIIB-9-433). In some aspects, the anti-FIXa antibody, or antigen binding portion thereof comprises VH CDR1, CDR2, and CDR3 sequences comprising SEQ ID NO: 822, SEQ ID NO: 867, and SEQ ID NO: 912, respectively, and/or VL CDR1, VL CDR2, and VL CDR3 sequences comprising SEQ ID NO: 957, SEQ ID NO: 1002, and SEQ ID NO: 1047, respectively (BIIB-9-619). In some aspects, the anti-FIXa antibody, or antigen binding portion thereof comprises VH CDR1, CDR2, and CDR3 sequences comprising SEQ ID NO: 843, SEQ ID NO: 888, and SEQ ID NO: 933, respectively, and/or VL CDR1, VL CDR2, and VL CDR3 sequences comprising SEQ ID NO: 950, SEQ ID NO: 995, and SEQ ID NO: 1040, respectively, respectively or (ii) the anti-FIXa antibody, or antigen binding portion thereof comprises VH CDR1, CDR2, and CDR3 sequences comprising SEQ ID NO: 844, SEQ ID NO: 889, and SEQ ID NO: 934, respectively, and/or VL CDR1, VL CDR2, and VL CDR3 sequences comprising SEQ ID NO: 950, SEQ ID NO: 995, and SEQ ID NO: 1040, respectively (BIIB-9-1335 and BIIB-9-1336). the anti-FIXa antibody, or antigen binding portion thereof comprises a VH and a VL, wherein the VH comprises an amino acid sequence which is at least about 80%, at least about 85%, at least about 90%, at least about 95%, at least about 96%, at least about 97%, at least about 98%, at least about 99%, or about 100% identical to the amino acid sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, and 181. In some aspects, the anti-FIXa antibody, or antigen binding portion thereof comprises a VH and a VL, wherein the VL comprises an amino acid sequence which is at least about 80%, at least about 85%, at least about 90%, at least about 95%, at least about 96%, at least about 97%, at least about 98%, at least about 99%, or about 100% identical to the amino acid sequence selected from the group consisting of SEQ ID NOs: 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, and 367. In some aspects, the anti-FIXa antibody, or antigen binding portion thereof comprises a VH and a VL, wherein the VH is derived from a germline sequence of VH1-46.0, VH1-46.4, VH1-46.5, VH1-46.7, VH1-46.9, VH1-69.9, VH3-07.0, VH3-21.0, VH3-21.2, VH3-23.0, VH3-23.1, VH4-31.0, VH4-34.0, VH4-39.0, VH4-39.2, VH4-39.3, VH4-39.5, VH4-39.6, VH4-39.8, VH4-59.6, VH4-0B.4, or VH4-0B.6. In some aspects, the anti-FIXa antibody, or antigen binding portion thereof comprises a VH and a VL, wherein the VL is derived from a germline sequence of VK1-05.0, VK1-05.6, VK1-05.9, VK1-05.21, VK1-12.0, VK1-12.3, VK1-33.0, VK1-33.1, VK1-33.2, VK1-33.8, VK1-33.10, VK1-39.0, VK1-39.6, VK2-28.0, VK2-28.1, VK3-11.0, VK3-11.2, VK3-11.6, VK3-11.10, VK3-11.14, VK3-15.0, VK3-15.6, VK3-15.8, VK3-15.11, VK3-15.20, VK3-15.26, VK3-20.0, VK3-20.4, VK3-20.5, VK3-20.8, or VK4-01.0. In some aspects, the anti-FIXa antibody, or antigen binding portion thereof comprises a VH and a VL, wherein (a1) VH and VL comprise SEQ ID NOs: 31 and 221, respectively (BIIB-9-484); (a2) VH and VL comprise SEQ ID NOs: 19 and 209, respectively (BIIB-9-440); (a3) VH and VL comprise SEQ ID NOs: 115 and 301, respectively (BIIB-9-882); (a4) VH and VL comprise SEQ ID NOs: 23 and 213, respectively (BIIB-9-460); (a5) VH and VL comprise SEQ ID NOs: 127 and 313, respectively (BIIB-9-433); (a6) VH and VL comprise SEQ ID NOs: 45 and 235, respectively (BIIB-9-619); (a7) VH and VL comprise SEQ ID NOs: 185 and 371, respectively (BIIB-9-578); (a8) VH and VL comprise SEQ ID NOs: 87 and 221, respectively (BIIB-9-1335); or, (a9) VH and VL comprise SEQ ID NOs: 89 and 221, respectively (BIIB-9-1336).

The present disclosure also provides an isolated antibody, or an antigen binding portion thereof, which specifically binds to FIXz (“anti-FIXz antibody or antigen binding portion thereof”), wherein the anti-FIXz antibody or antigen binding portion thereof preferentially binds to FIXz in the presence of free FIXa or FIXa-SM and/or the anti-FIXz antibody or antigen binding portion thereof binds to FIXz with a binding affinity higher than a binding affinity of the anti-FIXz antibody or antigen binding portion thereof to free FIXa or FIXa-SM. In some aspects, the anti-FIXz antibody, or antigen binding portion thereof cross-competes with a reference antibody selected from the group consisting of the antibodies in FIG. 3D. In some aspects, the anti-FIXz antibody, or antigen binding portion thereof binds to the same epitope as a reference antibody selected from the group consisting of the antibodies in FIG. 3D. In some aspects, such reference antibody is BIIB-9-578. In some aspects, the anti-FIXz antibody, or antigen binding portion thereof comprises CDR1, CDR2, and CDR3, wherein the CDR3 comprises a VH CDR3 selected from the group consisting of VH CDR3s in FIG. 3D or the VH CDR3 with one or two mutations. In some aspects, the CDR3 comprises ARDKYQDYSFDI (SEQ ID NO: 1355, BIIB-9-578). In some aspects, the anti-FIXz antibody, or antigen binding portion thereof comprises CDR1, CDR2, and CDR3, wherein the CDR1 comprises a VH CDR1 selected from the group consisting of VH CDR1s in FIG. 3D or the VH CDR1 with one or two mutations. In some aspects, the anti-FIXz antibody, or antigen binding portion thereof comprises VH CDR1, CDR2, and CDR3, wherein the CDR2 comprises a VH CDR2 selected from the group consisting of VH CDR2s in FIG. 3D or the VH CDR2 with one or two mutations. In some aspects, the anti-FIXz antibody, or antigen binding portion thereof comprises VL CDR1, CDR2, and CDR3, wherein the CDR1 comprises a VL CDR1 selected from the group consisting of VL CDR1s in FIG. 3D or the VL CDR1 with one or two mutations. In some aspects, the anti-FIXz antibody, or antigen binding portion thereof comprises VL CDR1, CDR2, and CDR3, wherein the CDR2 comprises a VL CDR2 selected from the group consisting of VL CDR2s in FIG. 3D or the VL CDR2 with one or two mutations. In some aspects, the anti-FIXz antibody, or antigen binding portion thereof comprises VL CDR1, CDR2, and CDR3, wherein the CDR3 comprises a VL CDR3 selected from the group consisting of VL CDR3s in FIG. 3D or the VL CDR3 with one or two mutations. In some aspects, the anti-FIX antibody is selected from the group consisting of an IgG1, an IgG2, an IgG3, an IgG4 or a variant thereof. In some aspects, the anti-FIX antibody is an IgG4 antibody. In some aspects, the anti-FIX antibody comprises an effectorless IgG4 Fc. In some aspects, the anti-FIX antibody, or antigen binding portion thereof comprises a heavy chain constant region. In some aspects, the anti-FIX antibody is a human antibody, an engineered antibody, or a humanized antibody. In some aspects, the anti-FIX antigen binding portion comprises an Fab, Fab′, F(ab′)2, Fv, or a single chain Fv (scFv).

The present disclosure also provides a bispecific molecule comprising an anti-FIX antibody or antigen binding portion thereof disclosed herein linked to a molecule having a second binding specificity. Also provides is a nucleic acid encoding the heavy and/or light chain variable region of an anti-FIX antibody, or antigen binding portion thereof disclosed herein or a bispecific molecule comprising an anti-FIX antibody, or antigen binding portion thereof disclosed herein. Also provided is an expression vector comprising a nucleic acid molecule encoding a disclosed herein. Also provided is a cell transformed with an expression vector disclosed herein. The present disclosure also provides an immunoconjugate comprising any antibody or antigen binding portion thereof disclosed herein or a bispecific molecule disclosed herein, linked to an agent. The present disclosure also provides a composition comprising (i) an antibody disclosed herein or an antigen binding portion thereof, a bispecific molecule disclosed herein, or an immunoconjugate disclosed herein, and (ii) a carrier. Also provide is a kit comprising (i) an antibody disclosed herein or an antigen binding portion thereof, a bispecific molecule disclosed herein, or an immunoconjugate disclosed herein, and (ii) instructions for use.

The present disclosure also provides a method of preparing an anti-FIX antibody, or antigen binding portion thereof, comprising expressing the antibody, or antigen binding portion thereof in a cell and isolating the antibody, or antigen binding portion thereof, from the cell. Also provided is a method of measuring a level of activated FIX in a subject in need thereof comprising contacting an anti-FIXa antibody disclosed herein or an antigen binding portion thereof, with a sample obtained from the subject under suitable conditions and measuring the binding of the anti-FIXa antibody or antigen binding portion thereof to FIXa in the sample. In some aspects, the sample is blood or serum.

The present disclosure provides an isolated antibody, or an antigen binding portion thereof, that specifically binds to factor X zymogen (FXz) (“anti-FXz antibody or antigen binding portion thereof”), wherein the anti-FXz antibody or antigen binding portion thereof preferentially binds to FXz in the presence of FXz and activated factor X (FXa). In some aspects, the anti-FXz antibody, or antigen binding portion thereof binds to FXz with a binding affinity higher than a binding affinity of the antibody or antigen binding portion thereof to FXa. Also provided is an isolated anti-FXz antibody, or antigen binding portion thereof, which binds to FXz with a binding affinity higher than a binding affinity of the antibody or antigen binding portion thereof to FXa. In some aspects, the anti-FXz antibody, or antigen binding portion thereof, binds to FXz with a K_{D} of about 100 nM or less, about 90 nM or less, about 80 nM or less, about 70 nM or less, about 60 nM or less, about 50 nM or less, about 40 nM or less, about 30 nM or less, about 20 nM or less, about 10 nM or less, about 9 nM or less, about 8 nM or less, about 7 nM or less, about 6 nM or less, about 5 nM or less, about 4 nM or less, about 3 nM or less, about 2 nM or less, about 1 nM or less as measured by BLI. In some aspects, the FXa is free FXa or FXa covalently linked to EGR-CMK (FIXa-SM). In some aspects, the FXz comprises non-activatable Factor X (FXn). In some aspects, the anti-FXz antibody, or antigen binding portion thereof cross-competes with a reference antibody selected from the group consisting of the antibodies in FIG. 12A and FIG. 12B. In some aspects, the anti-FXz antibody, or antigen binding portion thereof, binds to the same epitope as a reference antibody selected from the group consisting of the antibodies in FIG. 12A and FIG. 12B. In some aspects, the anti-FXz antibody, or antigen binding portion thereof, binds to the same epitope as a reference antibody selected from the group consisting of BIIB-12-915, BIIB-12-917, BIIB-12-932, and any combination thereof. In some aspects, the anti-FXz antibody, or antigen binding portion thereof, comprises CDR1, CDR2, and CDR3, wherein the CDR3 comprises a VH CDR3 selected from the group consisting of VH CDR3s in FIG. 12A and FIG. 12B or the VH CDR3 with one or two mutations. In some aspects, the anti-FXz antibody, or antigen binding portion thereof, comprises CDR1, CDR2, and CDR3, wherein the CDR3 comprises ARX₁X₂X₃RX₄X₅X₆X₇FDX₈ (SEQ ID NO: 766), wherein X₁ is G or L, X₂ is R or G, X₃ is F or Y, X₄ is P or G, X₅ is R or A, X₆ is G or S, X₇ is R or A, and X₈ is Y or I. In some aspects, the anti-FXz antibody, or antigen binding portion thereof, comprises CDR1, CDR2, and CDR3, wherein the CDR3 comprises ARGRFRPRGRFDY (SEQ ID NO: 1575, BIIB-12-917), ARLGYRGASAFDI (SEQ ID NO: 1589, BIIB-12-932), or ARVGGGYANP (SEQ ID NO: 1573, BIIB-12-915). In some aspects, the anti-FXz antibody, or antigen binding portion thereof, comprises VH CDR1, CDR2, and CDR3, wherein the CDR1 comprises a VH CDR1 selected from the group consisting of VH CDR1s in FIG. 12A and FIG. 12B or the VH CDR1 with one or two mutations. In some aspects, the anti-FXz antibody, or antigen binding portion thereof, comprises VH CDR1, CDR2, and CDR3, wherein the CDR2 comprises a VH CDR2 selected from the group consisting of VH CDR2s in FIG. 12A and FIG. 12B or the VH CDR2 with one or two mutations. In some aspects, the anti-FXz antibody, or antigen binding portion thereof, comprises VL CDR1, CDR2, and CDR3, wherein the CDR1 comprises a VL CDR1 selected from the group consisting of VL CDR1s in FIG. 12A and FIG. 12B or the VL CDR1 with one or two mutations. In some aspects, the anti-FXz antibody, or antigen binding portion thereof, comprises VL CDR1, CDR2, and CDR3, wherein the CDR2 comprises a VL CDR2 selected from the group consisting of VL CDR2s in FIG. 12A and FIG. 12B or the VL CDR2 with one or two mutations. In some aspects, the anti-FXz antibody, or antigen binding portion thereof, comprises VL CDR1, CDR2, and CDR3, wherein the CDR3 comprises a VL CDR3 selected from the group consisting of VL CDR3s in FIG. 12A and FIG. 12B or the VL CDR3 with one or two mutations.

The present disclosure also provides an isolated antibody, or antigen binding portion thereof, which specifically binds to FXz, comprising VH CDR1, CDR2, and CDR3 and VL CDR1, CDR2, and CDR3, wherein the VH CDR1, CDR2, and CDR3 and VL CDR1, CDR2, and CDR3 comprise VH CDR1, CDR2, and CDR3 and VL CDR1, CDR2, and CDR3 of FIG. 12A and FIG. 12B, respectively. In some aspects, the anti-FXz antibody, or antigen binding portion thereof, comprises VH CDR1, CDR2, and CDR3 sequences comprising SEQ ID NOs: 1393, 1483, or 1573, respectively, and/or VL CDR1, VL CDR2, and VL CDR3 sequences comprising SEQ ID NOs: 1663, 1753, or 1843, respectively (BIIB-12-915). In some aspects, the anti-FXz antibody, or antigen binding portion thereof, comprises VH CDR1, CDR2, and CDR3 sequences comprising SEQ ID NOs: 1395, 1485, or 1575, respectively, and/or VL CDR1, VL CDR2, and VL CDR3 sequences comprising SEQ ID NOs: 1665, 1755, or 1845, respectively (BIIB-12-917). In some aspects, the anti-FXz antibody, or antigen binding portion thereof, comprises VH CDR1, CDR2, and CDR3 sequences comprising SEQ ID NOs: 1409, 1499, or 1589, respectively, and/or VL CDR1, VL CDR2, and VL CDR3 sequences comprising SEQ ID NOs: 1679, 1769, or 1859, respectively (BIIB-12-932). In some aspects, the anti-FXz antibody, or antigen binding portion thereof, comprises VH and VL, wherein the VH comprises an amino acid sequence which is at least about 80%, at least about 85%, at least about 90%, at least about 95%, at least about 96%, at least about 97%, at least about 98%, at least about 99%, or about 100% identical to the amino acid sequence selected from the group consisting of SEQ ID NOs: 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, and 555. In some aspects, the anti-FXz antibody, or antigen binding portion thereof, comprises VH and VL, wherein the VL comprises an amino acid sequence which is at least about 80%, at least about 85%, at least about 90%, at least about 95%, at least about 96%, at least about 97%, at least about 98%, at least about 99%, or about 100% identical to the amino acid sequence selected from the group consisting of SEQ ID NOs: 565, 567, 569, 571, 573, 575, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, and 743. In some aspects, the anti-FXz antibody, or antigen binding portion thereof, comprises a VH and a VL, wherein the VH is derived from a germline sequence of VH1-18.0, VH1-18.1, VH1-18.8, VH1-46.0, VH1-46.4, VH1-46.5, VH1-46.6, VH1-46.7, VH1-46.8, VH1-46.9, VH3-21.0, VH3-23.0, VH3-23.2, VH3-23.6, VH3-30.0, VH4-31.5, VH4-39.0, VH4-39.5. VH4-0B.4, or VH5-51.1. In some aspects, the anti-FXz antibody, or antigen binding portion thereof, comprises a VH and a VL, wherein the VL is derived from a germline sequence of VK1-05.6, VK1-05.12, VK1-12.0, VK1-12.4, VK1-12.7, VK1-12.10, VK1-12.15, VK1-39.0, VK1-39.3, VK1-39.15, VK2-28.0, VK2-28.1, VK2-28.5, VK3-11.0, VK3-11.2, VK3-11.6, VK3-11.14, VK3-15.0, VK3-15.8, VK3-15.10, VK3-20.0, VK3-20.1, VK3-20.4, VK3-20.5, VK4-01.0, VK4-01.4, VK4-01.20. In some aspects, the anti-FX antibody, or antigen binding portion thereof, comprises VH and VL, wherein (b1) VH and VL comprise SEQ ID NOs: 423 and 611, respectively (BIIB-12-915); (b2) VH and VL comprise SEQ ID NOs: 427 and 615, respectively (BIIB-12-917); or (b3) VH and VL comprise SEQ ID NOs: 455 and 643, respectively (BIIB-12-932).

The present disclosure also provides an isolated antibody, or an antigen binding portion thereof, that specifically binds to activated factor X (FXa) (“anti-FXa antibody or antigen binding portion thereof”), wherein the anti-FXa antibody or antigen binding portion thereof preferentially binds to FXa in the presence of FXz and FXa and/or binds to FXa with a binding affinity higher than a binding affinity of the antibody or antigen binding portion thereof to FXz. In some aspects, the anti-FXa antibody, or antigen binding portion thereof, cross-competes with a reference antibody selected from the group consisting of the antibodies in FIG. 12C. In some aspects, the anti-FXa antibody, or antigen binding portion thereof, binds to the same epitope as a reference antibody selected from the group consisting of the antibodies in FIG. 12C. In some aspects, the anti-FXa antibody, or antigen binding portion thereof, binds to the same epitope as a reference antibody selected from the group consisting of BIIB-12-925. In some aspects, the anti-FXa antibody, or antigen binding portion thereof, comprises CDR1, CDR2, and CDR3, wherein the CDR3 comprises a VH CDR3 selected from the group consisting of VH CDR3s in FIG. 12C or the VH CDR3 with one or two mutations. In some aspects, the anti-FXa antibody, or antigen binding portion thereof, comprises CDR1, CDR2, and CDR3, wherein the CDR3 comprises AKGPRYYWYSWYFDL (SEQ ID NO: 1919, BIIB-12-925). In some aspects, the anti-FXa antibody, or antigen binding portion thereof, comprises VH CDR1, CDR2, and CDR3, wherein the CDR1 comprises a VH CDR1 selected from the group consisting of VH CDR1s in FIG. 12C or the VH CDR1 with one or two mutations. In some aspects, the anti-FXa antibody, or antigen binding portion thereof, comprises VH CDR1, CDR2, and CDR3, wherein the CDR2 comprises a VH CDR2 selected from the group consisting of VH CDR2s in FIG. 12C or the VH CDR2 with one or two mutations. In some aspects, the anti-FXa antibody, or antigen binding portion thereof, comprises VL CDR1, CDR2, and CDR3, wherein the CDR1 comprises a VL CDR1 selected from the group consisting of VL CDR1s in FIG. 12C or the VL CDR1 with one or two mutations. In some aspects, the anti-FXa antibody, or antigen binding portion thereof, comprises VL CDR1, CDR2, and CDR3, wherein the CDR2 comprises a VL CDR2 selected from the group consisting of VL CDR2s in FIG. 12C or the VL CDR2 with one or two mutations. In some aspects, the anti-FXa antibody, or antigen binding portion thereof, comprises VL CDR1, CDR2, and CDR3, wherein the CDR3 comprises a VL CDR3 selected from the group consisting of VL CDR3s in FIG. 12C or the VL CDR3 with one or two mutations.

The present disclosure also provides an isolated antibody, or antigen binding portion thereof, which specifically binds to FXa, comprising VH CDR1, CDR2, and CDR3 and VL CDR1, CDR2, and CDR3, wherein the VH CDR1, CDR2, and CDR3 and VL CDR1, CDR2, and CDR3 comprise VH CDR1, CDR2, and CDR3 and VL CDR1, CDR2, and CDR3 of FIG. 12C, respectively. In some aspects, the anti-FXa antibody, or antigen binding portion thereof, comprises VH CDR1, CDR2, and CDR3 sequences comprising SEQ ID NOs: 1911, 1915, or 1919, respectively, and/or VL CDR1, VL CDR2, and VL CDR3 sequences comprising SEQ ID NOs: 1923, 1927, or 1931, respectively (BIIB-12-925). In some aspects, the anti-FXa antibody, or antigen binding portion thereof, comprises VH and VL, wherein the VH and VL comprise SEQ ID NOs: 559 and 747, respectively (BIIB-12-925). In some aspects, the anti-FX antibody, or antigen binding portion thereof, is selected from the group consisting of an IgG1, an IgG2, an IgG3, an IgG4 or a variant thereof. In some aspects, the anti-FX antibody, or antigen binding portion thereof, is an IgG4 antibody. In some aspects, the anti-FX antibody, or antigen binding portion thereof, comprises an effectorless IgG4 Fc. In some aspects, the anti-FX antibody, or antigen binding portion thereof, comprises a heavy chain constant region. In some aspects, the anti-FX antibody is a human antibody, an engineered antibody, or a humanized antibody. In some aspects, the antigen binding portion thereof comprises an Fab, Fab′, F(ab′)2, Fv, or a single chain Fv (scFv). The present disclosure also provides a bispecific molecule comprising an anti-FX antibody disclosed herein linked to a molecule having a second binding specificity. Also provided is a nucleic acid encoding the heavy and/or light chain variable region of an anti-FX antibody disclosed herein, or antigen binding portion thereof, or a bispecific molecule comprising an anti-FX antibody or antigen binding portion thereof disclosed herein. Also provided is an expression vector comprising the nucleic acid molecule. Also provided is a cell transformed with the expression vector. Also provided is an immunoconjugate comprising the antibody, or antigen binding portion thereof, or the bispecific molecule, linked to an agent. Also provided is a composition comprising (i) the antibody, or antigen binding portion thereof, or the bispecific molecule, or the immunoconjugate, and (ii) a carrier. Also provided is a kit comprising (i) the antibody, or antigen binding portion thereof, or the bispecific molecule, or the immunoconjugate, and (ii) instructions for use. Also provided is a method of preparing an anti-FX antibody, or antigen binding portion thereof, comprising expressing the antibody, or antigen binding portion thereof, in a cell and isolating the antibody, or antigen binding portion thereof, from the cell.

The present disclosure also provides a method of measuring zymogen FX (FXz) in a subject in need thereof comprising contacting the anti-FX antibody, or antigen binding portion thereof, disclosed herein with a sample obtained from the subject under suitable conditions and measuring the binding of the anti-FX antibody or antigen binding portion thereof to FXz in the sample. In some aspects, the sample is blood or serum from the subject. The present disclosure also provides a bispecific molecule comprising (i) an anti-FIX antibody, or antigen binding portion thereof, disclosed herein, and (ii) an anti-FX antibody, or antigen binding portion thereof, disclosed herein. In some aspects, the bispecific molecule cross-competes with a reference bispecific antibody, wherein the reference bispecific antibody comprises a VH and a VL of an anti-FIX antibody selected from the group consisting of the anti-FIX antibodies in FIG. 3A, FIG. 3B, FIG. 3C, and FIG. 3D, and a VH and a VL of an anti-FX antibody selected from the group consisting of the anti-FX antibodies in FIG. 12A, FIG. 12B, and FIG. 12C. In some aspects, the bispecific molecule binds to the same epitope as a reference bispecific antibody, wherein the reference bispecific antibody comprises a VH and a VL of an anti-FIX antibody selected from the group consisting of the anti-FIX antibodies in FIG. 3A, FIG. 3B, FIG. 3C, and FIG. 3D and a VH and a VL of an anti-FX antibody selected from the group consisting of the anti-FX antibodies in FIG. 12A, FIG. 12B, and FIG. 12C. In some of aspects of the bispecific molecules disclosed herein (i) the anti-FIX antibody, or antigen binding portion thereof, comprises VH CDR1, CDR2, and CDR3 and VL CDR1, CDR2, and CDR3, wherein the VH CDR1, CDR2, and CDR3 and the VL CDR1, CDR2, and CDR3 are selected from the group consisting of VH CDR1s, VH CDR2s, and VH CDR3s and VL CDR1s, VL CDR2s, and VL CDR3s of the anti-FIX (BIIB-9) antibodies in FIG. 16A, FIG. 16B, FIG. 16C, and FIG. 16D; and, (ii) the anti-FX antibody, or antigen binding portion thereof, comprises VH CDR1, CDR2, and CDR3 and VL CDR1, CDR2, and CDR3, wherein the VH CDR1, CDR2, and CDR3 and the VL CDR1, CDR2, and CDR3 are selected from the group consisting of VH CDR1s, VH CDR2s, and VH CDR3s and VL CDR1s, VL CDR2s, and VL CDR3s of the anti-FX (BIIB-12) antibodies in FIG. 16A, FIG. 16B, FIG. 16C, and FIG. 16D. In some aspects of the bispecific molecule disclosed herein (a) the anti-FIX antibody, or antigen binding portion thereof, comprises (a1) VH CDR1, CDR2, and CDR3 sequences comprising SEQ ID NOs: 815, 860, or 905, respectively, and/or VL CDR1, VL CDR2, and VL CDR3 sequences comprising SEQ ID NOs: 950, 995, or 1040, respectively (BIIB-9-484); (a2) VH CDR1, CDR2, and CDR3 sequences comprising SEQ ID NOs: 822, 867, and 912, respectively, and/or VL CDR1, CDR2, and CDR3 sequences comprising SEQ ID NOs: 957, 1002, and 1047, respectively (BIIB-9-619); (a3) VH CDR1, CDR2, and CDR3 sequences comprising SEQ ID NOs: 1347, 1351, and 1355, respectively, and/or VL CDR1, CDR2, and CDR3 sequences comprising SEQ ID NOs: 1359, 1363, and 1367, respectively (BIIB-9-578); (a4) VH CDR1, CDR2, and CDR3 sequences comprising SEQ ID NOs: 843, 888, and 933, respectively, and/or VL CDR1, CDR2, and CDR3 sequences comprising SEQ ID NOs: 978, 1023, and 1068, respectively (BIIB-9-1335); or (a5) VH CDR1, CDR2, and CDR3 sequences comprising SEQ ID NOs: 844, 889, and 934, respectively, and/or VL CDR1, CDR2, and CDR3 sequences comprising SEQ ID NOs: 979, 1024, and 1069, respectively (BIIB-9-1336); and (b) the anti-FX antibody, or antigen binding portion thereof, comprises (b1) VH CDR1, CDR2, and CDR3 sequences comprising SEQ ID NOs: 1393, 1483, and 1573, respectively, and/or VL CDR1, CDR2, and CDR3 sequences comprising SEQ ID NOs: 1663, 1753, and 1843, respectively (BIIB-12-915); (b2) VH CDR1, CDR2, and CDR3 sequences comprising SEQ ID NOs: 1395, 1485, and 1575, respectively, and/or VL CDR1, CDR2, and CDR3 sequences comprising SEQ ID NOs: 1665, 1755, and 1845, respectively (BIIB-12-917); (b3) VH CDR1, CDR2, and CDR3 sequences comprising SEQ ID NOs: 1911, 1915, and 1919, respectively, and/or VL CDR1, CDR2, and CDR3 sequences comprising SEQ ID NOs: 1923, 1927, and 1931, respectively (BIIB-12-925); (b4) VH CDR1, CDR2, and CDR3 sequences comprising SEQ ID NOs: 1409, 1499, and 1589, respectively, and/or VL CDR1, CDR2, and CDR3 sequences comprising SEQ ID NOs: 1679, 1769, and 1859, respectively (BIIB-12-932); or (b5) VH CDR1, CDR2, and CDR3 sequences comprising SEQ ID NOs: 1433, 1523, and 1613, respectively, and/or VL CDR1, CDR2, and CDR3 sequences comprising SEQ ID NOs: 1703, 1793, and 1883, respectively (BIIB-12-1306). In some aspects of the bispecific molecule disclosed herein (a) the anti-FIX antibody, or antigen binding portion thereof, comprises (a1) a VH and a VL comprising SEQ ID NOs: 31 and 221, respectively (BIIB-9-484); (a2) a VH and a VL comprising SEQ ID NOs: 45 and 235, respectively (BIIB-9-619); (a3) a VH and a VL comprising SEQ ID NOs: 185 and 371, respectively (BIIB-9-578); (a4) a VH and a VL comprising SEQ ID NOs: 87 and 221, respectively (BIIB-9-1335); or (a5) a VH and a VL comprising SEQ ID NOs: 89 and 221, respectively (BIIB-9-1336); and, (b) the anti-FX antibody, or antigen binding portion thereof, comprises (b1) a VH and a VL comprising SEQ ID NOs: 423 and 611, respectively (BIIB-12-915); (b2) a VH and a VL comprising SEQ ID NOs: 427 and 615, respectively (BIIB-12-917); (b3) a VH and a VL comprising SEQ ID NOs: 559 and 747, respectively (BIIB-12-925); (b4) a VH and VL comprising SEQ ID NOs: 455 and 643, respectively (BIIB-12-932); or, (b5) a VH and a VL comprising SEQ ID NOs: 503 and 691, respectively (BIIB-12-1306). In some aspects of the bispecific molecule disclosed herein (i) the anti-FIX antibody, or antigen binding portion thereof, comprises a VH and a VL comprising SEQ ID NOs: 31 and 221, respectively (BIIB-9-484); and the anti-FX antibody, or antigen binding portion thereof, comprises a VH and a VL comprising SEQ ID NOs: 423 and 611, respectively (BIIB-12-915); or (ii) the anti-FIX antibody, or antigen binding portion thereof, comprises a VH and a VL comprising SEQ ID NOs: 31 and 221, respectively (BIIB-9-484); and the anti-FX antibody, or antigen binding portion thereof, comprises a VH and a VL comprising SEQ ID NOs: 427 and 615, respectively (BIIB-12-917); or (iii) the anti-FIX antibody, or antigen binding portion thereof, comprises a VH and a VL comprising SEQ ID NOs: 31 and 221, respectively (BIIB-9-484); and the anti-FX antibody, or antigen binding portion thereof, comprises a VH and a VL comprising SEQ ID NOs: 559 and 747, respectively (BIIB-12-925); or (iv) the anti-FIX antibody, or antigen binding portion thereof, comprises a VH and a VL comprising SEQ ID NOs: 31 and 221, respectively (BIIB-9-484); and the anti-FX antibody, or antigen binding portion thereof, comprises a VH and a VL comprising SEQ ID NOs: 455 and 643, respectively (BIIB-12-932); or, (v) the anti-FIX antibody, or antigen binding portion thereof, comprises a VH and a VL comprising SEQ ID NOs: 185 and 371, respectively (BIIB-9-578); and the anti-FX antibody, or antigen binding portion thereof, comprises a VH and a VL comprising SEQ ID NOs: 423 and 611, respectively (BIIB-12-915); or, (vi) the anti-FIX antibody, or antigen binding portion thereof, comprises a VH and a VL comprising SEQ ID NOs: 185 and 371, respectively (BIIB-9-578); and the anti-FX antibody, or antigen binding portion thereof, comprises a VH and a VL comprising SEQ ID NOs: 427 and 615, respectively (BIIB-12-917); or, (vii) the anti-FIX antibody, or antigen binding portion thereof, comprises a VH and a VL comprising SEQ ID NOs: 45 and 235, respectively (BIIB-9-619); and the anti-FX antibody, or antigen binding portion thereof, comprises a VH and a VL comprising SEQ ID NOs: 427 and 615, respectively (BIIB-12-917); or, (viii) the anti-FIX antibody, or antigen binding portion thereof, comprises a VH and a VL comprising SEQ ID NOs: 45 and 235, respectively (BIIB-9-619); and the anti-FX antibody, or antigen binding portion thereof, comprises a VH and a VL comprising SEQ ID NOs: 559 and 747, respectively (BIIB-12-925). In some aspects, the bispecific molecule functionally mimics activated factor VIII (FVIIIa) cofactor in at least one FVIIIa activity assay. In some aspects, the FVIIIa activity assay is selected from a chromogenic FXa generation assay, a one-stage clotting assay, or the combination thereof. In some aspects, the FVIIIa activity achieves at least 10%, 20%, 30%, 35%, 40%, 45% 50%, 60%, 70%, 80%, 90%, 100%, 110%, 120%, 130%, 140%, 150%, 160%, 170%, 180%, 190%, or 200% of the activity otherwise achieved by FVIII in the same assay. In some aspects, the bispecific molecule is capable of generating thrombin from prothrombin, fibrin from fibrinogen, and/or fibrin clot in vitro or in vivo. In some aspects, the bispecific molecule concurrently binds to both FIXa and FX, as determined by BLI. In some aspects, the bispecific molecule is of the IgG isotype. In some aspects, the IgG isotype is of the IgG1 subclass. In some aspects, the IgG isotype is of the IgG4 subclass. In some aspects, the bispecific molecule is of a bispecific IgG format and is selected from the group consisting of the antibodies in TABLE 2. In some aspects, the bispecific molecule is of a bispecific heterodimeric format. In some aspects, the bispecific molecule comprises two different heavy chains and two different light chains. In some aspects, the bispecific molecule comprises two identical light chains and two different heavy chains. In some aspects, the bispecific molecule is capable of controlling or reducing the incidence of bleeding episodes in a subject having hemophilia. In some aspects, the bispecific molecule is capable of maintaining homeostasis or in a subject having hemophilia. In some aspects, the bispecific molecule is capable of providing routine prophylaxis in a subject having hemophilia. In some aspects, the subject has developed or is expected to develop neutralizing antibodies against Factor VIII.

The present disclosure also provides an immunoconjugate comprising a bispecific molecule disclosed herein linked to an agent, e.g., a therapeutic agent. Also provided is a composition comprising (i) a bispecific molecule disclosed herein or an immunoconjugate comprising the bispecific molecule, and (ii) a carrier. Also provide is a kit comprising (i) a bispecific molecule disclosed herein or an immunoconjugate comprising the bispecific molecule, and (ii) instructions for use. Also provided is a nucleic acid sequence encoding the bispecific molecule disclosed herein. Also provided are a vector comprising the nucleic acid, and a host cell comprising the vector. In some aspects, the host cell is a prokaryotic cell, a eukaryotic cell, a protist cell, an animal cell, a plant cell, a fungal cell, a yeast cell, an Sf9 cell, a mammalian cell, an avian cell, an insect cell, a CHO cell, a HEK cell, or a COS cell. Also provided is a method of producing a bispecific molecule disclosed herein, comprising culturing a host cell disclosed herein under conditions that allow the expression of the bispecific molecule. In some aspects, the method of producing a bispecific molecule disclosed herein further comprises using conditions that enhance heterodimerization.

The present disclosure also provides a method of promoting FX activation in a subject in need thereof, comprising administering to the subject a therapeutically effective amount of a bispecific molecule disclosed herein, or an immunoconjugate, composition, nucleic acid, vector, or host cell disclosed herein which comprises or encodes a bispecific molecule disclosed herein.

The present disclosure also provides a method of reducing the frequency or degree of a bleeding episode in a subject in need thereof, comprising administering to the subject an effective amount of a bispecific molecule disclosed herein or an immunoconjugate, composition, nucleic acid, vector, or host cell disclosed herein which comprises or encodes a bispecific molecule disclosed herein. In some aspects, the subject has developed or has a tendency to develop an inhibitor against Factor VIII (“FVIII”). In some aspects, the inhibitor against FVIII is a neutralizing antibody against FVIII. In some aspects, the bleeding episode is the result of hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, bleeding in the illiopsoas sheath, or any combinations thereof.

The present disclosure also provides a method of treating a blood coagulation disorder in a subject in need thereof, comprising administering to the subject an effective amount of a bispecific molecule disclosed herein or an immunoconjugate, composition, nucleic acid, vector, or host cell disclosed herein which comprises or encodes a bispecific molecule disclosed herein. In some aspects, the blood coagulation disorder is hemophilia A or hemophilia B. In some aspects, the subject is a human subject. In some aspects, the subject is undergoing or has undergone FVIII replacement therapy. In some aspects, the bispecific molecule is administered in combination with a hemophilia therapy. In some aspects, the hemophilia therapy is a FVIII replacement therapy. In some aspects, the bispecific molecule, immunoconjugate, composition, nucleic acid, vector, or host cell is administered before, during or after administration of the hemophilia therapy. In some aspects, the bispecific molecule, immunoconjugate, composition, nucleic acid, vector, or host cell is administered intravenously or subcutaneously. In some aspects, administration of the bispecific molecule, immunoconjugate, composition, nucleic acid, vector, or host cell reduces the frequency of break-through bleeding episodes, spontaneous bleeding episodes, or acute bleeding. In some aspects, administration of the bispecific molecule, immunoconjugate, composition, nucleic acid, vector, or host cell reduces the annualized bleed rate by 5%, 10%, 20%, 30%, or 50%.

The present disclosure also provides an anti-FIXa antibody, or antigen binding portion thereof, which binds to the same epitope as BIIB-9-1336. Also provides is an anti-FIXa antibody, or antigen binding portion thereof, which binds to an epitope overlapping BIIB-9-1336 epitope.

The present disclosure also provides an anti-FIXa antibody, or antigen binding portion thereof, which binds to an epitope region comprising at least one amino acid located between chymotrypsinogen numbering positions (i) 91 and 101, (ii) 125 and 128, (iii) 165 and 179, or (iv) 232 and 241 in the sequence of the heavy chain of FIXa. Also provided is an anti-FIXa antibody, or antigen binding portion thereof, which binds to an epitope comprising at least one of chymotrypsinogen numbering amino acid residues H91, H92, N93, H101, D125, K126, E127, Y128, R165, Y177, N178, N179, S232, R233, Y234, V235, N236, W237, E240, and K241 of the sequence of the heavy chain of FIXa. In some aspects, the epitope comprises chymotrypsinogen numbering amino acid residues N93, R165, N178, and R233 of the sequence of the heavy chain of FIXa. In other aspects, the epitope comprises chymotrypsinogen numbering amino acid residues H91, H92, N93, H101, D125, K126, E127, Y128, R165, Y177, N178, N179, S232, R233, Y234, V235, N236, W237, E240, and K241 of the sequence of the heavy chain of FIXa. In some aspects, the epitope does not comprise at least one of chymotrypsinogen numbering amino acid residues N100, K132, Y137, R170, T172, F174, T175, H185, E202, and G205 of the sequence of the heavy chain of FIXa. In some aspects, the epitope does not comprise chymotrypsinogen numbering amino acid residues N100, K132, Y137, R170, T172, F174, T175, H185, E202, and G205 of the sequence of the heavy chain of FIXa. In some aspects, the anti-FIXa antibody, or antigen binding portion thereof, binds to at least one amino acid residue in the light chain of FIXa (SEQ ID NO:756). In some aspects, the amino acid residue in the light chain of FIXa (SEQ ID NO:756) is K100. In some aspects, the epitope overlaps the binding site of FVIIIa to FIXa. In some aspects, the anti-FIXa antibody, or antigen binding portion thereof, cross-competes with FVIIIa for binding to FIXa. In some aspects, the anti-FIXa antibody, or antigen binding portion thereof, blocks binding of FVIIIa to FIXa.

In some aspects, the anti-FIXa antibody, or antigen binding portion thereof, disclosed herein which specifically binds to FIXa, comprises VH CDR1, CDR2, and CDR3 and VL CDR1, CDR2, and CDR3, wherein (i) the VH CDR1 comprises a VH CDR1 selected from the group consisting of VH CDR1s in TABLE 7 or the VH CDR1 with one or two mutations; and/or, (ii) the VH CDR2 comprises a VH CDR2 selected from the group consisting of VH CDR2s in TABLE 7 or the VH CDR2 with one or two mutations; and/or, (iii) the VH CDR3 comprises a VH CDR3 selected from the group consisting of VH CDR3s in TABLE 7 or the VH CDR3 with one or two mutations; and/or, (iv) the VL CDR1 comprises a VL CDR1 selected from the group consisting of VL CDR1s in TABLE 7 or the VL CDR1 with one or two mutations; and/or, (v) the VL CDR2 comprises a VL CDR2 selected from the group consisting of VL CDR2s in TABLE 7 or the VL CDR2 with one or two mutations; and/or, (vi) the VL CDR3 comprises a VL CDR3 selected from the group consisting of VL CDR3s in TABLE 7 or the VL CDR3 with one or two mutations.

In some aspects, the anti-FIXa antibody, or antigen binding portion thereof, disclosed herein which specifically binds to FIXa, comprises VH CDR1, CDR2, and CDR3 and VL CDR1, CDR2, and CDR3, wherein the VH CDR3 comprises the amino acid sequence ARDXIGGYAGYYGMDV (SEQ ID NO: 2196), wherein X₁ is L or V. In some aspects, (i) the VH CDR1 comprises the amino acid sequence FTFX₁SX₂X₃MX₄ (SEQ ID NO: 2194), wherein X₁ is S, G or E, X₂ is Y or F, X₃ is S, E, G, or D, and X₄ is N, V, A, or T; and/or (ii) the VH CDR2 comprises the amino acid sequence X₅ISX₆X₇X₈X₉X₁₀IYYADSVKG (SEQ ID NO: 2195), wherein X₅ is S, A, Y, or G, X₆ is S or A, X₇ is S, A, or G, X₈ is S, G, or D, X₉ is S, T, or G, and X₁₀ is Y or T.

In some aspects, the anti-FIXa antibody, or antigen binding portion thereof, disclosed herein which specifically binds to FIXa, comprises VL CDR1, CDR2 and CDR3, wherein the VL CDR3 comprises the amino acid sequence QQYANFPYT (SEQ ID NO:2168). In some aspects, (i) the VL CDR1 comprises the amino acid sequence QASQDIANYLN (SEQ ID NO:2116); and/or, (ii) the VL CDR2 comprises the amino acid sequence DASNLET (SEQ ID NO:2142).

In some aspects, the anti-FIXa antibody, or antigen binding portion thereof, disclosed herein which specifically binds to FIXa, comprises (i) VH CDR1, CDR2, and CDR3, wherein VH CDR1 is selected from SEQ ID NOs: 2038 to 2047, VH CDR2 is selected from SEQ ID NOs: 2064 to 2073, and VH CDR3 is selected from SEQ ID NOs: 2090 to 2099, and/or (ii) VL CDR1, CDR2, and CDR3, wherein VL CDR1 is selected from SEQ ID NOs: 2116 to 2125, a VL CDR2 is selected from SEQ ID NOs: 2142 to 2151, and a VL CDR3 is selected from SEQ ID NOs: 2168 to 2177.

In some aspects, the anti-FIXa antibody, or antigen binding portion thereof, disclosed herein which specifically binds to FIXa, comprises VH CDR1, CDR2, and CDR3 and VL CDR1, CDR2, and CDR3, wherein the VH CDR3 comprises the amino acid sequence X₁RDVX₂GYAGX₃YGMDV (SEQ ID NO: 2198), wherein X₁ is A or V, X₂ is G or S, and X₃ is Y or F. In some aspects, (i) the VH CDR1 comprises the amino acid sequence FTFGSYDMN (SEQ ID NO: 2048); and/or (ii) the VH CDR2 comprises the amino acid sequence SISX₁X₂X₃SYIX₄YAX₅SVKG (SEQ ID NO: 2197), wherein X₁ is S or D, X₂ is G or S, X₃ is E or A, X₄ is Y or A, and X₅ is E or D.

In some aspects, the anti-FIXa antibody, or antigen binding portion thereof, disclosed herein which specifically binds to FIXa, comprises VH CDR1, CDR2, and CDR3 and VL CDR1, CDR2, and CDR3, wherein the VL CDR3 comprises the amino acid sequence X₁QYAX₂FPYT (SEQ ID NO: 2201), wherein X₁ is Q or S, and X₂ is N or R. In some aspects, the anti-FIXa antibody, or antigen binding portion thereof, disclosed herein which specifically binds to FIXa, comprises VL CDR1, CDR2 and CDR3, wherein (i) the VL CDR1 comprises the amino acid sequence X₁AX₂X₃X₄IX₅X₆YLN (SEQ ID NO: 2199), wherein X₁ is Q, G, or E, X₂ is S or N, X₃ is Q or E, X₄ is D or Y, X₅ is A or S, X₆ is N or D; and/or (ii) the VL CDR2 comprises the amino acid sequence DAX₇NLX₈X₉ (SEQ ID NO: 2200), wherein X₇ is S or A, X₈ is E, H or Q, and X₉ is T or Y.

In some aspects, the anti-FIXa antibody, or antigen binding portion thereof, disclosed herein which specifically binds to FIXa, comprises (i) VH CDR1, CDR2, and CDR3, wherein VH CDR1 is selected from SEQ ID NOs: 2048 to 2052, VH CDR2 is selected from SEQ ID NOs: 2074 to 2078, and VH CDR3 is selected from SEQ ID NOs: 2100 to 2104, and/or (ii) VL CDR1, CDR2, and CDR3 wherein VL CDR1 is selected from SEQ ID NOs: 2126 to 2130, VL CDR2 is selected from SEQ ID NOs: 2152 to 2156, and VL CDR3 is selected from SEQ ID NOs: 2178 to 2182.

In some aspects, the anti-FIXa antibody, or antigen binding portion thereof, disclosed herein which specifically binds to FIXa, comprises VH CDR1, CDR2, and CDR3 and VL CDR1, CDR2, and CDR3, wherein the VH CDR3 comprises the amino acid sequence ARDGPX₁X₂X₃DYYMDV (SEQ ID NO: 2204), wherein X₁ is R or Q, X₂ is V, D, L or E, and X₃ is S or V. In some aspects, the anti-FIXa antibody, or antigen binding portion thereof, disclosed herein which specifically binds to FIXa, comprises VH CDR1, CDR2 and CDR3, wherein (i) the VH CDR1 comprises the amino acid sequence YTFX₁X₂YX₃MH (SEQ ID NO: 2202), wherein X₁ is T or H, X₂ is S, G, or H, and X₃ is Y or P; and/or (ii) the VH CDR2 comprises the amino acid sequence X₄INPSX₅GX₆TX₇YAQKFQG (SEQ ID NO: 2203), wherein X₄ is I or S, X₅ is G or R, X₆ is S or R, and X₇ is S or E.

In some aspects, the anti-FIXa antibody, or antigen binding portion thereof, disclosed herein which specifically binds to FIXa, comprises VH CDR1, CDR2, and CDR3 and VL CDR1, CDR2, and CDR3, wherein the VL CDR3 comprises the amino acid sequence QQRDNWPFT (SEQ ID NO:2116). In some aspects, the anti-FIXa antibody, or antigen binding portion thereof, disclosed herein which specifically binds to FIXa, comprises VL CDR1, CDR2 and CDR3, wherein (i) the VL CDR1 comprises the amino acid sequence RASQSVSSYLA (SEQ ID NO:2116); and/or, (ii) the VL CDR2 comprises the amino acid sequence DASNRAT (SEQ ID NO:2116).

In some aspects, the anti-FIXa antibody, or antigen binding portion thereof, disclosed herein which specifically binds to FIXa, comprises (i) VH CDR1, CDR2, and CDR3, wherein VH CDR1 is selected from SEQ ID NOs: 2053 to 2057, VH CDR2 is selected from SEQ ID NOs: 2079 to 2083, and VH CDR3 is selected from SEQ ID NOs: 2105 to 2109, and/or (ii) VL CDR1, CDR2, and CDR3, wherein VL CDR1 is selected from SEQ ID NOs: 2131 to 2135, VL CDR2 is selected from SEQ ID NOs: 2157 to 2161, and VL CDR3 is selected from SEQ ID NOs: 2183 to 2187.

In some aspects, the anti-FIXa antibody, or antigen binding portion thereof, disclosed herein which specifically binds to FIXa, comprises VH CDR1, CDR2, and CDR3 and VL CDR1, CDR2, and CDR3, wherein the VH CDR3 comprises the amino acid sequence ARDKYQDYSX₁DI (SEQ ID NO: 2207), wherein X₁ is F or V. In some aspects, the anti-FIXa antibody, or antigen binding portion thereof, disclosed herein which specifically binds to FIXa, comprises VH CDR1, CDR2 and CDR3, wherein (i) the VH CDR1 comprises the amino acid sequence GSIX₁SX₂X₃YX₄WX₅(SEQ ID NO: 2205), wherein X₁ is S or A, X₂ is S, T, G, or V, X₃ is S or A, X₄ is Y or A, and X₅ is G, V, N, or S; and/or (ii) the VH CDR2 comprises the amino acid sequence X₆IX₇X₈X₉GX₁₀TX₁₁YNPSLKS (SEQ ID NO: 2206), wherein X₆ is S or Y, X₇ is S, Y, R, T or Q, X₈ is Y, G, P or A, X₉ is S or Q, X₁₀ is S or K, and X₁₁ is Y or Q.

In some aspects, the anti-FIXa antibody, or antigen binding portion thereof, disclosed herein which specifically binds to FIXa, comprises VL CDR1, CDR2 and CDR3, wherein the VL CDR3 comprises the amino acid sequence QQANFLPFT (SEQ ID NO:2188). In some aspects, the anti-FIXa antibody, or antigen binding portion thereof, disclosed herein which specifically binds to FIXa, comprises VL CDR1, CDR2 and CDR3, wherein (i) the VL CDR1 comprises the amino acid sequence RASQGIDSWLA (SEQ ID NO:2136); and/or, (ii) the VL CDR2 comprises the amino acid sequence AASSLQS (SEQ ID NO:2162).

In some aspects, the anti-FIXa antibody, or antigen binding portion thereof, disclosed herein which specifically binds to FIXa, comprises (i) VH CDR1, CDR2, and CDR3, wherein VH CDR1 is selected from SEQ ID NOs: 2058 to 2063, VH CDR2 is selected from SEQ ID NOs: 2084 to 2089, and VH CDR3 is selected from SEQ ID NOs: 2110 to 2115, and/or (ii) VL CDR1, CDR2, and CDR3, wherein VL CDR1 is selected from SEQ ID NOs: 2136 to 2141, VL CDR2 is selected from SEQ ID NOs: 2162 to 2167, and VL CDR3 is selected from SEQ ID NOs: 2188 to 2193.

In some aspects, the anti-FIXa antibody, or antigen binding portion thereof, disclosed herein which specifically binds to FIXa, comprises a VH and a VL, wherein (i) the VH comprises an amino acid sequence which is at least about 80%, at least about 85%, at least about 90%, at least about 95%, at least about 96%, at least about 97%, at least about 98%, at least about 99%, or about 100% identical to the amino acid sequence selected from the group consisting of SEQ ID NOs: 1935, 1939, 1943, 1947, 1951, 1955, 1959, 1963, 1967, 1971, 1975, 1979, 1983, 1987, 1991, 1995, 1999, 2003, 2007, 2011, 2015, 2019, 2023, 2027, 2031, and 2035; and/or (ii) the VL comprises an amino acid sequence which is at least about 80%, at least about 85%, at least about 90%, at least about 95%, at least about 96%, at least about 97%, at least about 98%, at least about 99%, or about 100% identical to the amino acid sequence selected from the group consisting of SEQ ID NOs: 1937, 1941, 1945, 1949, 1953, 1957, 1961, 1965, 1969, 1973, 1977, 1981, 1985, 1989, 1993, 1997, 2001, 2005, 2009, 2013, 2017, 2021, 2025, 2029, 2033, and 2037.

In some aspects, the anti-FIXa antibody, or antigen binding portion thereof, disclosed herein which specifically binds to FIXa, comprises a VH and a VL, wherein (a1) the VH and the VL comprise SEQ ID NOs: 1935 and 1937, respectively (BIIB-9-3595); (a2) the VH and the VL comprise SEQ ID NOs: 1939 and 1941, respectively (BIIB-9-3601); (a3) the VH and the VL comprise SEQ ID NOs: 1943 and 1945, respectively (BIIB-9-3604); (a4) the VH and the VL comprise SEQ ID NOs: 1947 and 1949, respectively (BIIB-9-3617); (a5) the VH and the VL comprise SEQ ID NOs: 1951 and 1953, respectively (BIIB-9-3618); (a6) the VH and the VL comprise SEQ ID NOs: 1955 and 1957, respectively (BIIB-9-3621); (a7) the VH and the VL comprise SEQ ID NOs: 1959 and 1961, respectively (BIIB-9-3647); (a8) the VH and the VL comprise SEQ ID NOs: 1963 and 1965, respectively (BIIB-9-3649); (a9) the VH and the VL comprise SEQ ID NOs: 1967 and 1969, respectively (BIIB-9-3650); (a10) the VH and the VL comprise SEQ ID NOs: 1971 and 1973, respectively (BIIB-9-3654); (a11) the VH and the VL comprise SEQ ID NOs: 1975 and 1977, respectively (BIIB-9-3753); (a12) the VH and the VL comprise SEQ ID NOs: 1979 and 1981, respectively (BIIB-9-3754); (a13) the VH and the VL comprise SEQ ID NOs: 1983 and 1985, respectively (BIIB-9-3756); (a14) the VH and the VL comprise SEQ ID NOs: 1987 and 1989, respectively (BIIB-9-3764); (a15) the VH and the VL comprise SEQ ID NOs: 1991 and 1993, respectively (BIIB-9-3766); (a16) the VH and the VL comprise SEQ ID NOs: 1995 and 1997, respectively (BIIB-9-3707); (a17) the VH and the VL comprise SEQ ID NOs: 1999 and 2001, respectively (BIIB-9-3709); (a18) the VH and the VL comprise SEQ ID NOs: 2003 and 2005, respectively (BIIB-9-3720); (a19) the VH and the VL comprise SEQ ID NOs: 2007 and 2009, respectively (BIIB-9-3727); (a20) the VH and the VL comprise SEQ ID NOs: 2011 and 2013, respectively (BIIB-9-3745); (a21) the VH and the VL comprise SEQ ID NOs: 2015 and 2017, respectively (BIIB-9-3780); (a22) the VH and the VL comprise SEQ ID NOs: 2019 and 2021, respectively (BIIB-9-3675); (a23) the VH and the VL comprise SEQ ID NOs: 2023 and 2025, respectively (BIIB-9-3681); (a24) the VH and the VL comprise SEQ ID NOs: 2027 and 2029, respectively (BIIB-9-3684); (a25) the VH and the VL comprise SEQ ID NOs: 2031 and 2033, respectively (BIIB-9-3698); or, (a26) the VH and the VL comprise SEQ ID NOs: 2035 and 2037, respectively (BIIB-9-3704).

### EMBODIMENTS

Embodiment 1. An isolated antibody, or an antigen binding portion thereof, that specifically binds to activated factor IX (FIXa) (“anti-FIXa antibody or antigen binding portion thereof”), wherein the anti-FIXa antibody or antigen binding portion thereof preferentially binds to FIXa in the presence of FIXa and factor IX zymogen (FIXz).

Embodiment 2. The anti-FIXa antibody, or antigen binding portion thereof, of embodiment 1, which binds to FIXa with a binding affinity higher than a binding affinity of the anti-FIXa antibody or antigen binding portion thereof to FIXz.

Embodiment 3. An isolated anti-FIXa antibody, or antigen binding portion thereof, which binds to FIXa with a binding affinity higher than a binding affinity of the anti-FIXa antibody or antigen binding portion thereof to FIXz.

Embodiment 4. The anti-FIXa antibody, or antigen binding portion thereof, of any preceding embodiments, which binds to FIXa with a K_{D} of about 100 nM or less, about 90 nM or less, about 80 nM or less, about 70 nM or less, about 60 nM or less, about 50 nM or less, about 40 nM or less, about 30 nM or less, about 20 nM or less, about 10 nM or less, about 1 nM or less as determined by a Bio-Layer Interferometry (BLI) assay.

Embodiment 5. The anti-FIXa antibody, or antigen binding portion thereof, of any preceding embodiments, wherein the FIXa is free FIXa, FIXa in a tenase complex, or FIXa covalently linked to EGR-CMK (FIXa-SM).

Embodiment 6. The anti-FIXa antibody, or antigen binding portion thereof, of any preceding embodiments, wherein the FIXz comprises non-activatable Factor IX (FIXn).

Embodiment 7. The anti-FIXa antibody, or antigen binding portion thereof, of any preceding embodiments, which cross-competes with a reference antibody selected from the group consisting of the antibodies in FIG. 3A, FIG. 3B, and FIG. 3C.

Embodiment 8. The anti-FIXa antibody, or antigen binding portion thereof, of any preceding embodiments, which binds to the same epitope as a reference antibody selected from the group consisting of the antibodies in FIG. 3A, FIG. 3B, and FIG. 3C.

Embodiment 9. The anti-FIXa antibody, or antigen binding portion thereof, of embodiment 7 or 8, wherein the reference antibody is selected from BIIB-9-484, BIIB-9-440, BIIB-9-882, BIIB-9-460, BIIB-9-433, and any combination thereof.

Embodiment 10. The anti-FIXa antibody, or antigen binding portion thereof, of any preceding embodiments, which preferentially binds to FIXa-SM compared to free FIXa or FIXz and/or binds to FIXa-SM with a binding affinity higher than a binding affinity of the anti-FIXa antibody or antigen binding portion thereof to free FIXa or FIXz.

Embodiment 11. The anti-FIXa antibody, or antigen binding portion thereof, of any preceding embodiments, which cross-competes with a reference antibody selected from the group consisting of the antibodies in FIG. 3A.

Embodiment 12. The anti-FIXa antibody, or antigen binding portion thereof, of any preceding embodiments, which binds to the same epitope as a reference antibody selected from the group consisting of the antibodies in FIG. 3A.

Embodiment 13. The anti-FIXa antibody, or antigen binding portion thereof, of embodiment 11 or 12, wherein the reference antibody is selected from BIIB-9-484, BIIB-9-440, BIIB-9-460, and any combination thereof.

Embodiment 14. The anti-FIXa antibody, or antigen binding portion thereof, of any preceding embodiments, which preferentially binds to free FIXa compared to FIXa-SM or FIXz and/or binds to free FIXa with a binding affinity higher than a binding affinity of the anti-FIXa antibody or antigen binding portion thereof to FIXa-SM or FIXz.

Embodiment 15. The anti-FIXa antibody, or antigen binding portion thereof, of any preceding embodiments, which cross-competes with a reference antibody selected from the group consisting of the antibodies in FIG. 3B.

Embodiment 16. The anti-FIXa antibody, or antigen binding portion thereof, of any preceding embodiments, which binds to the same epitope as a reference antibody selected from the group consisting of the antibodies in FIG. 3B.

Embodiment 17. The anti-FIXa antibody, or antigen binding portion thereof, of any preceding embodiments, which preferentially binds to free FIXa or FIXa-SM compared to FIXz and/or binds to free FIXa or FIXa-SM with a binding affinity higher than a binding affinity of the anti-FIXa antibody or antigen binding portion thereof to FIXz.

Embodiment 18. The anti-FIXa antibody, or antigen binding portion thereof, of any preceding embodiments, which cross-competes with a reference antibody selected from the group consisting of the antibodies in FIG. 3C. 19. Embodiment The anti-FIXa antibody, or antigen binding portion thereof, of any preceding embodiments, which binds to the same epitope as a reference antibody selected from the group consisting of the antibodies in FIG. 3C.

Embodiment 20. The anti-FIXa antibody, or antigen binding portion thereof, of embodiment 18 or 19, wherein the reference antibody is selected from BIIB-9-882, BIIB-9-433, and the combination thereof.

21. The anti-FIXa antibody, or antigen binding portion thereof, of any preceding embodiments, which comprises CDR1, CDR2, and CDR3, wherein the CDR3 comprises a VH CDR3 selected from the group consisting of VH CDR3s in FIG. 3A, FIG. 3B, and FIG. 3C or the VH CDR3 with one or two mutations.

Embodiment 22. The anti-FIXa antibody, or antigen binding portion thereof, of any one of embodiments 1 to 21, which comprises CDR1, CDR2, and CDR3, wherein the CDR3 comprises ARDX₁X₂X₃X₄X₅X₆YYX₇MDV (SEQ ID NO:753), wherein X₁ is V or G, X₂ is G or V, X₃ is G or R, X₄ is Y or V, X₅ is A or S, X₆ is G or D, X₇ is G or none.

Embodiment 23. The anti-FIXa antibody, or antigen binding portion thereof, of embodiment 22, wherein the CDR3 comprises ARDVGGYAGYYGMDV (SEQ ID NO: 905, BIIB-9-484, 1335, 1336), ARDISTDGESSLYYYMDV (SEQ ID NO: 901, BIIB-9-460), ARGPTDSSGYLDMDV (SEQ ID NO: 1186, BIIB-9-882), or ARDGPRVSDYY MDV (SEQ ID NO: 912, BIIB-9-619).

Embodiment 24. The anti-FIXa antibody, or antigen binding portion thereof, of any preceding embodiments, which comprises CDR1, CDR2, and CDR3, wherein the CDR1 comprises a VH CDR1 selected from the group consisting of VH CDR1s in FIG. 3A, FIG. 3B, and FIG. 3C or the VH CDR1 with one or two mutations.

Embodiment 25. The anti-FIXa antibody, or antigen binding portion thereof, of any preceding embodiments, which comprises VH CDR1, CDR2, and CDR3, wherein the CDR2 comprises a VH CDR2 selected from the group consisting of VH CDR2s in FIG. 3A, FIG. 3B, and FIG. 3C or the VH CDR2 with one or two mutations.

Embodiment 26. The anti-FIXa antibody, or antigen binding portion thereof, of any preceding embodiments, which comprises VL CDR1, CDR2, and CDR3, wherein the CDR1 comprises a VL CDR1 selected from the group consisting of VL CDR1s in FIG. 3A, FIG. 3B, and FIG. 3C or the VL CDR1 with one or two mutations.

Embodiment 27. The anti-FIXa antibody, or antigen binding portion thereof, of any preceding embodiments, which comprises VL CDR1, CDR2, and CDR3, wherein the CDR2 comprises a VL CDR2 selected from the group consisting of VL CDR2s in FIG. 3A, FIG. 3B, and FIG. 3C or the VL CDR2 with one or two mutations.

Embodiment 28. The anti-FIXa antibody, or antigen binding portion thereof, of any preceding embodiments, which comprises VL CDR1, CDR2, and CDR3, wherein the CDR3 comprises a VL CDR3 selected from the group consisting of VL CDR3s in FIG. 3A, FIG. 3B, and FIG. 3C or the VL CDR3 with one or two mutations.

Embodiment 29. An isolated anti-FIXa antibody, or antigen binding portion thereof, which specifically binds to FIXa, comprising VH CDR1, CDR2, and CDR3 and VL CDR1, CDR2, and CDR3, wherein the VH CDR1, CDR2, and CDR3 and the VL CDR1, CDR2, and CDR3 comprise VH CDR1, VH CDR2, and VH CDR3 and VL CDR1, CDR2, and CDR3 of FIG. 3A, FIG. 3B, and FIG. 3C, respectively.

Embodiment 30. The anti-FIXa antibody, or antigen binding portion thereof, of embodiment 29, wherein the anti-FIXa antibody or antigen binding portion thereof comprises VH CDR1, CDR2, and CDR3 sequences comprising SEQ ID NOs: 815, 860, and 905, respectively, and/or VL CDR1, CDR2, and CDR3 sequences comprising SEQ ID NOs: 950, 995, and 1040, respectively (BIIB-9-484).

Embodiment 31. The anti-FIXa antibody, or antigen binding portion thereof, of embodiment 29, wherein (a1) the antibody comprises VH CDR1, CDR2, and CDR3 sequences comprising SEQ ID NO: 809, SEQ ID NO: 854, and SEQ ID NO: 899, respectively, and/or VL CDR1, VL CDR2, and VL CDR3 sequences comprising SEQ ID NO: 944, SEQ ID NO: 989, and SEQ ID NO: 1034, respectively (BIIB-9-440); (a2) the antibody comprises VH CDR1, CDR2, and CDR3 sequences comprising SEQ ID NO: 1102, SEQ ID NO: 1144, and SEQ ID NO: 1186, respectively, and/or VL CDR1, VL CDR2, and VL CDR3 sequences comprising SEQ ID NO: 1228, SEQ ID NO: 1270, and SEQ ID NO: 1312, respectively (BIIB-9-882); (a3) the antibody comprises VH CDR1, CDR2, and CDR3 sequences comprising SEQ ID NO: 811, SEQ ID NO: 856, and SEQ ID NO: 901, respectively, and/or VL CDR1, VL CDR2, and VL CDR3 sequences comprising SEQ ID NO: 946, SEQ ID NO: 991, and SEQ ID NO: 1036, respectively (BIIB-9-460); or, (a4) the antibody comprises VH CDR1, CDR2, and CDR3 sequences comprising SEQ ID NO: 1108, SEQ ID NO: 1150, and SEQ ID NO: 1192, respectively, and/or VL CDR1, VL CDR2, and VL CDR3 sequences comprising SEQ ID NO: 1234, SEQ ID NO: 1276, and SEQ ID NO: 1318, respectively (BIIB-9-433).

Embodiment 32. The anti-FIXa antibody, or antigen binding portion thereof, of embodiment 29, wherein the antibody comprises VH CDR1, CDR2, and CDR3 sequences comprising SEQ ID NO: 822, SEQ ID NO: 867, and SEQ ID NO: 912, respectively, and/or VL CDR1, VL CDR2, and VL CDR3 sequences comprising SEQ ID NO: 957, SEQ ID NO: 1002, and SEQ ID NO: 1047, respectively (BIIB-9-619).

Embodiment 33. The anti-FIXa antibody, or antigen binding portion thereof, of embodiment 29, wherein (i) the antibody comprises VH CDR1, CDR2, and CDR3 sequences comprising SEQ ID NO: 843, SEQ ID NO: 888, and SEQ ID NO: 933, respectively, and/or VL CDR1, VL CDR2, and VL CDR3 sequences comprising SEQ ID NO: 950, SEQ ID NO: 995, and SEQ ID NO: 1040, respectively, respectively or (ii) the antibody comprises VH CDR1, CDR2, and CDR3 sequences comprising SEQ ID NO: 844, SEQ ID NO: 889, and SEQ ID NO: 934, respectively, and/or VL CDR1, VL CDR2, and VL CDR3 sequences comprising SEQ ID NO: 950, SEQ ID NO: 995, and SEQ ID NO: 1040, respectively (BIIB-9-1335 and BIIB-9-1336).

Embodiment 34. The anti-FIXa antibody, or antigen binding portion thereof, of any preceding embodiments, comprising VH and VL, wherein the VH comprises an amino acid sequence which is at least about 80%, at least about 85%, at least about 90%, at least about 95%, at least about 96%, at least about 97%, at least about 98%, at least about 99%, or about 100% identical to the amino acid sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, and 181.

Embodiment 35. The anti-FIXa antibody, or antigen binding portion thereof of any preceding embodiments, comprising VH and VL, wherein the VL comprises an amino acid sequence which is at least about 80%, at least about 85%, at least about 90%, at least about 95%, at least about 96%, at least about 97%, at least about 98%, at least about 99%, or about 100% identical to the amino acid sequence selected from the group consisting of SEQ ID NOs: 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, and 367.

Embodiment 36. The anti-FIXa antibody, or antigen binding portion thereof of any preceding embodiments, which comprises a VH and a VL, wherein the VH is derived from a germline sequence of VH1-46.0, VH1-46.4, VH1-46.5, VH1-46.7, VH1-46.9, VH1-69.9, VH3-07.0, VH3-21.0, VH3-21.2, VH3-23.0, VH3-23.1, VH4-31.0, VH4-34.0, VH4-39.0, VH4-39.2, VH4-39.3, VH4-39.5, VH4-39.6, VH4-39.8, VH4-59.6, VH4-0B.4, or VH4-0B.6.

Embodiment 37. The anti-FIXa antibody, or antigen binding portion thereof of any preceding embodiments, which comprises a VH and a VL, wherein the VL is derived from a germline sequence of VK1-05.0, VK1-05.6, VK1-05.9, VK1-05.21, VK1-12.0, VK1-12.3, VK1-33.0, VK1-33.1, VK1-33.2, VK1-33.8, VK1-33.10, VK1-39.0, VK1-39.6, VK2-28.0, VK2-28.1, VK3-11.0, VK3-11.2, VK3-11.6, VK3-11.10, VK3-11.14, VK3-15.0, VK3-15.6, VK3-15.8, VK3-15.11, VK3-15.20, VK3-15.26, VK3-20.0, VK3-20.4, VK3-20.5, VK3-20.8, or VK4-01.0.

Embodiment 38. The anti-FIXa antibody, or antigen binding portion thereof of any preceding embodiments, comprising VH and VL, wherein (a1) VH and VL comprise SEQ ID NOs: 31 and 221, respectively (BIIB-9-484); (a2) VH and VL comprise SEQ ID NOs: 19 and 209, respectively (BIIB-9-440); (a3) VH and VL comprise SEQ ID NOs: 115 and 301, respectively (BIIB-9-882); (a4) VH and VL comprise SEQ ID NOs: 23 and 213, respectively (BIIB-9-460); (a5) VH and VL comprise SEQ ID NOs: 127 and 313, respectively (BIIB-9-433); (a6) VH and VL comprise SEQ ID NOs: 45 and 235, respectively (BIIB-9-619); (a7) VH and VL comprise SEQ ID NOs: 185 and 371, respectively (BIIB-9-578); (a8) VH and VL comprise SEQ ID NOs: 87 and 221, respectively (BIIB-9-1335); or, (a9) VH and VL comprise SEQ ID NOs: 89 and 221, respectively (BIIB-9-1336).

Embodiment 39. An isolated antibody, or an antigen binding portion thereof, which specifically binds to FIXz (“anti-FIXz antibody or antigen binding portion thereof”), wherein the anti-FIXz antibody or antigen binding portion thereof preferentially binds to FIXz in the presence of free FIXa or FIXa-SM and/or the anti-FIXz antibody or antigen binding portion thereof binds to FIXz with a binding affinity higher than a binding affinity of the anti-FIXz antibody or antigen binding portion thereof to free FIXa or FIXa-SM.

Embodiment 40. The anti-FIXz antibody, or antigen binding portion thereof, of any preceding embodiments, which cross-competes with a reference antibody selected from the group consisting of the antibodies in FIG. 3D.

Embodiment 41. The anti-FIXz antibody, or antigen binding portion thereof, of any preceding embodiments, which binds to the same epitope as a reference antibody selected from the group consisting of the antibodies in FIG. 3D.

Embodiment 42. The anti-FIXz antibody, or antigen binding portion thereof, of embodiment 40 or 41, wherein the reference antibody is BIIB-9-578.

Embodiment 43. The anti-FIXz antibody, or antigen binding portion thereof, of any preceding embodiments, which comprises CDR1, CDR2, and CDR3, wherein the CDR3 comprises a VH CDR3 selected from the group consisting of VH CDR3s in FIG. 3D or the VH CDR3 with one or two mutations.

Embodiment 44. The anti-FIXz antibody, or antigen binding portion thereof, of embodiment 43, wherein the CDR3 comprises ARDKYQDYSFDI (SEQ ID NO: 1355, BIIB-9-578).

Embodiment 45. The anti-FIXz antibody, or antigen binding portion thereof, of any preceding embodiments, which comprises CDR1, CDR2, and CDR3, wherein the CDR1 comprises a VH CDR1 selected from the group consisting of VH CDR1s in FIG. 3D or the VH CDR1 with one or two mutations.

Embodiment 46. The anti-FIXz antibody, or antigen binding portion thereof, of any preceding embodiments, which comprises VH CDR1, CDR2, and CDR3, wherein the CDR2 comprises a VH CDR2 selected from the group consisting of VH CDR2s in FIG. 3D or the VH CDR2 with one or two mutations.

Embodiment 47. The anti-FIXz antibody, or antigen binding portion thereof, of any preceding embodiments, which comprises VL CDR1, CDR2, and CDR3, wherein the CDR1 comprises a VL CDR1 selected from the group consisting of VL CDR1s in FIG. 3D or the VL CDR1 with one or two mutations.

Embodiment 48. The anti-FIXz antibody, or antigen binding portion thereof, of any preceding embodiments, which comprises VL CDR1, CDR2, and CDR3, wherein the CDR2 comprises a VL CDR2 selected from the group consisting of VL CDR2s in FIG. 3D or the VL CDR2 with one or two mutations.

Embodiment 49. The anti-FIXz antibody, or antigen binding portion thereof, of any preceding embodiments, which comprises VL CDR1, CDR2, and CDR3, wherein the CDR3 comprises a VL CDR3 selected from the group consisting of VL CDR3s in FIG. 3D or the VL CDR3 with one or two mutations.

Embodiment 50. The anti-FIX antibody, or antigen binding portion thereof, of any one of the preceding embodiments and embodiments 169 to 204, wherein the antibody is selected from the group consisting of an IgG1, an IgG2, an IgG3, an IgG4 or a variant thereof.

Embodiment 51. The anti-FIX antibody, or antigen binding portion thereof, of embodiment 50, wherein the antibody is an IgG4 antibody.

Embodiment 52. The anti-FIX antibody, or antigen binding portion thereof, of embodiment 50, wherein the antibody comprises an effectorless IgG4 Fc.

Embodiment 53. The anti-FIX antibody, or antigen binding portion thereof, of any one of the preceding embodiments and embodiments 169 to 204, comprises a heavy chain constant region.

Embodiment 54. The anti-FIX antibody of any one of the preceding embodiments and embodiments 169 to 204, wherein the antibody is a human antibody, an engineered antibody, or a humanized antibody.

Embodiment 55. The anti-FIX antigen binding portion thereof of any one of the preceding embodiments and embodiments 169 to 204, wherein the antigen binding portion thereof comprises an Fab, Fab′, F(ab′)2, Fv, or a single chain Fv (scFv).

Embodiment 56. A bispecific molecule comprising the anti-FIX antibody or antigen binding portion thereof of any one of the preceding embodiments and embodiments 169 to 204 linked to a molecule having a second binding specificity.

Embodiment 57. A nucleic acid encoding the heavy and/or light chain variable region of the anti-FIX antibody, or antigen binding portion thereof, of any one of embodiments 1-55 and embodiments 169 to 204 or the bispecific molecule of embodiment 56.

Embodiment 58. An expression vector comprising the nucleic acid molecule of embodiment 57.

Embodiment 59. A cell transformed with an expression vector of embodiment 58.

Embodiment 60. An immunoconjugate comprising the antibody or antigen binding portion thereof according to any one of embodiments 1 to 55 and embodiments 169 to 204 or the bispecific molecule of embodiment 56, linked to an agent.

Embodiment 61. A composition comprising the antibody, or antigen binding portion thereof of any one of embodiment 1 to 55 and embodiments 169 to 204, the bispecific molecule of embodiment 56, or the immunoconjugate of embodiment 60, and a carrier.

Embodiment 62. A kit comprising the antibody, or antigen binding portion thereof of any one of embodiments 1 to 55 and embodiments 169 to 204, the bispecific molecule of embodiment 56, or the immunoconjugate, of embodiment 60 and instructions for use.

Embodiment 63. A method of preparing an anti-FIX antibody, or antigen binding portion thereof, comprising expressing the antibody, or antigen binding portion thereof, in the cell of embodiment 59 and isolating the antibody, or antigen binding portion thereof, from the cell.

Embodiment 64. A method of measuring a level of activated FIX in a subject in need thereof comprising contacting the anti-FIXa antibody, or antigen binding portion thereof of any one of embodiments 1 to 55 and embodiments 169 to 204, with a sample obtained from the subject under suitable conditions and measuring the binding of the anti-FIXa antibody or antigen binding portion thereof to FIXa in the sample.

Embodiment 65. The method of embodiment 64, wherein the sample is blood or serum.

Embodiment 66. An isolated antibody, or an antigen binding portion thereof, that specifically binds to factor X zymogen (FXz) (“anti-FXz antibody or antigen binding portion thereof”), wherein the anti-FXz antibody or antigen binding portion thereof preferentially binds to FXz in the presence of FXz and activated factor X (FXa).

Embodiment 67. The anti-FXz antibody, or antigen binding portion thereof, of embodiment 66, which binds to FXz with a binding affinity higher than a binding affinity of the antibody or antigen binding portion thereof to FXa.

Embodiment 68. An isolated anti-FXz antibody, or antigen binding portion thereof, which binds to FXz with a binding affinity higher than a binding affinity of the antibody or antigen binding portion thereof to FXa.

Embodiment 69. The anti-FXz antibody, or antigen binding portion thereof, of any one of embodiments 66 to 68, which binds to FXz with a KD of about 100 nM or less, about 90 nM or less, about 80 nM or less, about 70 nM or less, about 60 nM or less, about 50 nM or less, about 40 nM or less, about 30 nM or less, about 20 nM or less, about 10 nM or less, about 1 nM or less as measured by BLI.

Embodiment 70. The anti-FXz antibody, or antigen binding portion thereof, of any one of embodiments 66 to 69, wherein the FXa is free FXa or FXa covalently linked to EGR-CMK (FIXa-SM).

Embodiment 71. The anti-FXz antibody, or antigen binding portion thereof, of any one of embodiments 66 to 70, wherein the FXz comprises non-activatable Factor X (FXn).

Embodiment 72. The anti-FXz antibody, or antigen binding portion thereof, of any one of embodiments 66 to 71, which cross-competes with a reference antibody selected from the group consisting of the antibodies in FIG. 12A and FIG. 12B.

Embodiment 73. The anti-FXz antibody, or antigen binding portion thereof, of any one of embodiments 66 to 72, which binds to the same epitope as a reference antibody selected from the group consisting of the antibodies in FIG. 12A and FIG. 12B.

Embodiment 74. The anti-FXz antibody, or antigen binding portion thereof, of embodiment 73, which binds to the same epitope as a reference antibody selected from the group consisting of BIIB-12-915, BIIB-12-917, BIIB-12-932, and any combination thereof.

Embodiment 75. The anti-FXz antibody, or antigen binding portion thereof, of any one of embodiments 66 to 74, which comprises CDR1, CDR2, and CDR3, wherein the CDR3 comprises a VH CDR3 selected from the group consisting of VH CDR3s in FIG. 12A and FIG. 12B or the VH CDR3 with one or two mutations.

Embodiment 76. The anti-FXz antibody, or antigen binding portion thereof, of any one of embodiments 66 to 75, which comprises CDR1, CDR2, and CDR3, wherein the CDR3 comprises ARX₁X₂X₃RX₄X₅X₆X₇FDX₈ (SEQ ID NO: 766), wherein X₁ is G or L, X₂ is R or G, X₃ is F or Y, X₄ is P or G, X₅ is R or A, X₆ is G or S, X₇ is R or A, and X₈ is Y or I.

Embodiment 77. The anti-FXz antibody, or antigen binding portion thereof, of any one of embodiments 66 to 76, which comprises CDR1, CDR2, and CDR3, wherein the CDR3 comprises ARGRFRPRGRFDY (SEQ ID NO: 1575, BIIB-12-917), ARLGYRGASAFDI (SEQ ID NO: 1589, BIIB-12-932), or ARVGGGYANP (SEQ ID NO: 1573, BIIB-12-915).

Embodiment 78. The anti-FXz antibody, or antigen binding portion thereof, of any one of embodiments 66 to 77, which comprises VH CDR1, CDR2, and CDR3, wherein the CDR1 comprises a VH CDR1 selected from the group consisting of VH CDR1s in FIG. 12A and FIG. 12B or the VH CDR1 with one or two mutations.

Embodiment 79. The anti-FXz antibody, or antigen binding portion thereof, of any one of embodiments 66 to 78, which comprises VH CDR1, CDR2, and CDR3, wherein the CDR2 comprises a VH CDR2 selected from the group consisting of VH CDR2s in FIG. 12A and FIG. 12B or the VH CDR2 with one or two mutations.

Embodiment 80. The anti-FXz antibody, or antigen binding portion thereof, of any one of embodiments 66 to 79, which comprises VL CDR1, CDR2, and CDR3, wherein the CDR1 comprises a VL CDR1 selected from the group consisting of VL CDR1s in FIG. 12A and FIG. 12B or the VL CDR1 with one or two mutations.

Embodiment 81. The anti-FXz antibody, or antigen binding portion thereof, of any one of embodiments 66 to 80, which comprises VL CDR1, CDR2, and CDR3, wherein the CDR2 comprises a VL CDR2 selected from the group consisting of VL CDR2s in FIG. 12A and FIG. 12B or the VL CDR2 with one or two mutations.

Embodiment 82. The anti-FXz antibody, or antigen binding portion thereof, of any one of embodiments 66 to 81, which comprises VL CDR1, CDR2, and CDR3, wherein the CDR3 comprises a VL CDR3 selected from the group consisting of VL CDR3s in FIG. 12A and FIG. 12B or the VL CDR3 with one or two mutations.

Embodiment 83. An isolated antibody, or antigen binding portion thereof, which specifically binds to FXz, comprising VH CDR1, CDR2, and CDR3 and VL CDR1, CDR2, and CDR3, wherein the VH CDR1, CDR2, and CDR3 and VL CDR1, CDR2, and CDR3 comprise VH CDR1, CDR2, and CDR3 and VL CDR1, CDR2, and CDR3 of FIG. 12A and FIG. 12B, respectively.

Embodiment 84. The anti-FXz antibody, or antigen binding portion thereof, of embodiment 83, wherein the antibody comprises VH CDR1, CDR2, and CDR3 sequences comprising SEQ ID NOs: 1393, 1483, or 1573, respectively, and/or VL CDR1, VL CDR2, and VL CDR3 sequences comprising SEQ ID NOs: 1663, 1753, or 1843, respectively (BIIB-12-915).

Embodiment 85. The anti-FXz antibody, or antigen binding portion thereof, of embodiment 83, wherein the antibody comprises VH CDR1, CDR2, and CDR3 sequences comprising SEQ ID NOs: 1395, 1485, or 1575, respectively, and/or VL CDR1, VL CDR2, and VL CDR3 sequences comprising SEQ ID NOs: 1665, 1755, or 1845, respectively (BIIB-12-917).

Embodiment 86. The anti-FXz antibody, or antigen binding portion thereof, of embodiment 83, wherein the antibody comprises VH CDR1, CDR2, and CDR3 sequences comprising SEQ ID NOs: 1409, 1499, or 1589, respectively, and/or VL CDR1, VL CDR2, and VL CDR3 sequences comprising SEQ ID NOs: 1679, 1769, or 1859, respectively (BIIB-12-932).

Embodiment 87. The anti-FXz antibody, or antigen binding portion thereof, of any one of embodiments 66 to 86, comprising VH and VL, wherein the VH comprises an amino acid sequence which is at least about 80%, at least about 85%, at least about 90%, at least about 95%, at least about 96%, at least about 97%, at least about 98%, at least about 99%, or about 100% identical to the amino acid sequence selected from the group consisting of SEQ ID NOs: 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495,497, 499, 501, 503, 505, 507, 509, 511, 513, 515, 517, 519, 521, 523, 525, 527, 529, 531, 533, 535, 537, 539, 541, 543, 545, 547, 549, 551, 553, and 555.

Embodiment 88. The anti-FXz antibody, or antigen binding portion thereof of any one of embodiments 66 to 87, comprising VH and VL, wherein the VL comprises an amino acid sequence which is at least about 80%, at least about 85%, at least about 90%, at least about 95%, at least about 96%, at least about 97%, at least about 98%, at least about 99%, or about 100% identical to the amino acid sequence selected from the group consisting of SEQ ID NOs: 565, 567, 569, 571, 573, 575, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, and 743.

Embodiment 89. The anti-FXz antibody, or antigen binding portion thereof, of any one of embodiments 66 to 88, which comprises a VH and a VL, wherein the VH is derived from a germline sequence of VH1-18.0, VH1-18.1, VH1-18.8, VH1-46.0, VH1-46.4, VH1-46.5, VH1-46.6, VH1-46.7, VH1-46.8, VH1-46.9, VH3-21.0, VH3-23.0, VH3-23.2, VH3-23.6, VH3-30.0, VH4-31.5, VH4-39.0, VH4-39.5. VH4-0B.4, or VH5-51.1.

Embodiment 90. The anti-FXz antibody, or antigen binding portion thereof, of any one of embodiments 66 to 89, which comprises a VH and a VL, wherein the VL is derived from a germline sequence of VK1-05.6, VK1-05.12, VK1-12.0, VK1-12.4, VK1-12.7, VK1-12.10, VK1-12.15, VK1-39.0, VK1-39.3, VK1-39.15, VK2-28.0, VK2-28.1, VK2-28.5, VK3-11.0, VK3-11.2, VK3-11.6, VK3-11.14, VK3-15.0, VK3-15.8, VK3-15.10, VK3-20.0, VK3-20.1, VK3-20.4, VK3-20.5, VK4-01.0, VK4-01.4, VK4-01.20.

Embodiment 91. The anti-FXz antibody, or antigen binding portion thereof, of any one of embodiments 66 to 90, comprising VH and VL, wherein

(b1) VH and VL comprise SEQ ID NOs: 423 and 611, respectively (BIIB-12-915);
(b2) VH and VL comprise SEQ ID NOs: 427 and 615, respectively (BIIB-12-917); or,
(b3) VH and VL comprise SEQ ID NOs: 455 and 643, respectively (BIIB-12-932).

Embodiment 92. An isolated antibody, or an antigen binding portion thereof, that specifically binds to activated factor X (FXa) (“anti-FXa antibody or antigen binding portion thereof”), wherein the anti-FXa antibody or antigen binding portion thereof preferentially binds to FXa in the presence of FXz and FXa and/or binds to FXa with a binding affinity higher than a binding affinity of the antibody or antigen binding portion thereof to FXz.

Embodiment 93. The anti-FXa antibody, or antigen binding portion thereof, of embodiment 92, which cross-competes with a reference antibody selected from the group consisting of the antibodies in FIG. 12C.

Embodiment 94. The anti-FXa antibody, or antigen binding portion thereof, of embodiment 92 or 93, which binds to the same epitope as a reference antibody selected from the group consisting of the antibodies in FIG. 12C.

Embodiment 95. The anti-FXa antibody, or antigen binding portion thereof, of embodiment 94, which binds to the same epitope as a reference antibody selected from the group consisting of BIIB-12-925.

Embodiment 96. The anti-FXa antibody, or antigen binding portion thereof, of any one of embodiments 92 to 95, which comprises CDR1, CDR2, and CDR3, wherein the CDR3 comprises a VH CDR3 selected from the group consisting of VH CDR3s in FIG. 12C or the VH CDR3 with one or two mutations.

Embodiment 97. The anti-FXa antibody, or antigen binding portion thereof, of any one of embodiments 92 to 96, which comprises CDR1, CDR2, and CDR3, wherein the CDR3 comprises AKGPRYYWYSWYFDL (SEQ ID NO: 1919, BIIB-12-925).

Embodiment 98. The anti-FXa antibody, or antigen binding portion thereof, of any one of embodiments 92 to 97, which comprises VH CDR1, CDR2, and CDR3, wherein the CDR1 comprises a VH CDR1 selected from the group consisting of VH CDR1s in FIG. 12C or the VH CDR1 with one or two mutations.

Embodiment 99. The anti-FXa antibody, or antigen binding portion thereof, of any one of embodiments 92 to 98, which comprises VH CDR1, CDR2, and CDR3, wherein the CDR2 comprises a VH CDR2 selected from the group consisting of VH CDR2s in FIG. 12C or the VH CDR2 with one or two mutations.

Embodiment 100. The anti-FXa antibody, or antigen binding portion thereof, of any one of embodiments 92 to 99, which comprises VL CDR1, CDR2, and CDR3, wherein the CDR1 comprises a VL CDR1 selected from the group consisting of VL CDR1s in FIG. 12C or the VL CDR1 with one or two mutations.

Embodiment 101. The anti-FXa antibody, or antigen binding portion thereof, of any one of embodiments 92 to 100, which comprises VL CDR1, CDR2, and CDR3, wherein the CDR2 comprises a VL CDR2 selected from the group consisting of VL CDR2s in FIG. 12C or the VL CDR2 with one or two mutations.

Embodiment 102. The anti-FXa antibody, or antigen binding portion thereof, of any one of embodiments 92 to 101, which comprises VL CDR1, CDR2, and CDR3, wherein the CDR3 comprises a VL CDR3 selected from the group consisting of VL CDR3s in FIG. 12C or the VL CDR3 with one or two mutations.

Embodiment 103. An isolated antibody, or antigen binding portion thereof, which specifically binds to FXa, comprising VH CDR1, CDR2, and CDR3 and VL CDR1, CDR2, and CDR3, wherein the VH CDR1, CDR2, and CDR3 and VL CDR1, CDR2, and CDR3 comprise VH CDR1, CDR2, and CDR3 and VL CDR1, CDR2, and CDR3 of FIG. 12C, respectively.

Embodiment 104. The anti-FXa antibody, or antigen binding portion thereof, of embodiment 103, wherein the antibody comprises VH CDR1, CDR2, and CDR3 sequences comprising SEQ ID NOs: 1911, 1915, or 1919, respectively, and/or VL CDR1, VL CDR2, and VL CDR3 sequences comprising SEQ ID NOs: 1923, 1927, or 1931, respectively (BIIB-12-925).

Embodiment 105. The anti-FXa antibody, or antigen binding portion thereof, of any one of embodiments 92 to 104, comprising VH and VL, wherein the VH and VL comprise SEQ ID NOs: 559 and 747, respectively (BIIB-12-925).

Embodiment 106. The anti-FX antibody, or antigen binding portion thereof, of any one of embodiments 66 to 105, wherein the antibody is selected from the group consisting of an IgG1, an IgG2, an IgG3, an IgG4 or a variant thereof.

Embodiment 107. The anti-FX antibody, or antigen binding portion thereof, of embodiment 106, wherein the antibody is an IgG4 antibody.

Embodiment 108. The anti-FX antibody, or antigen binding portion thereof, of embodiment 106, wherein the antibody comprises an effectorless IgG4 Fc.

Embodiment 109. The anti-FX antibody, or antigen binding portion thereof, of any one of embodiments 66 to 108, which comprises a heavy chain constant region.

Embodiment 110. The anti-FX antibody of any one of embodiments 66 to 109, wherein the antibody is a human antibody, an engineered antibody, or a humanized antibody.

Embodiment 111. The anti-FX antigen binding portion thereof of any one of embodiments 66 to 110, wherein the antigen binding portion thereof comprises an Fab, Fab′, F(ab′)2, Fv, or a single chain Fv (scFv).

Embodiment 112. A bispecific molecule comprising the anti-FX antibody of any one of embodiments 66 to 111 linked to a molecule having a second binding specificity.

Embodiment 113. A nucleic acid encoding the heavy and/or light chain variable region of the antibody, or antigen binding portion thereof, of any one of embodiments 66-111 or the bispecific molecule of embodiment 112.

Embodiment 114. An expression vector comprising the nucleic acid molecule of embodiment 113.

Embodiment 115. A cell transformed with an expression vector of embodiment 114.

Embodiment 116. An immunoconjugate comprising the antibody, or antigen binding portion thereof, of any one of embodiments 66-111 or the bispecific molecule of embodiment 112, linked to an agent.

Embodiment 117. A composition comprising the antibody, or antigen binding portion thereof, of any one of embodiments 66-111 or the bispecific molecule of embodiment 112, or the immunoconjugate of embodiment 116, and a carrier.

Embodiment 118. A kit comprising the antibody, or antigen binding portion thereof, of any one of embodiments 66-111 or the bispecific molecule of embodiment 112, or the immunoconjugate of embodiment 116, and instructions for use.

Embodiment 119. A method of preparing an anti-FX antibody, or antigen binding portion thereof, comprising expressing the antibody, or antigen binding portion thereof, in the cell of embodiment 115 and isolating the antibody, or antigen binding portion thereof, from the cell.

Embodiment 120. A method of measuring zymogen FX (FXz) in a subject in need thereof comprising contacting the anti-FX antibody, or antigen binding portion thereof, of any one of embodiments 66 to 111 with a sample obtained from the subject under suitable conditions and measuring the binding of the anti-FX antibody or antigen binding portion thereof to FXz in the sample.

Embodiment 121. The method of embodiment 120, wherein the sample is blood or serum from the subject.

Embodiment 122. A bispecific molecule comprising the anti-FIX antibody, or antigen binding portion thereof, of any one of embodiments 1 to 55 and embodiments 169 to 204 and (ii) the anti-FX antibody, or antigen binding portion thereof, of any one of embodiments 66 to 111.

Embodiment 123. The bispecific molecule of embodiment 122, which cross-competes with a reference bispecific antibody, wherein the reference bispecific antibody comprises a VH and a VL of an anti-FIX antibody selected from the group consisting of the anti-FIX antibodies in FIG. 3A, FIG. 3B, FIG. 3C, and FIG. 3D and a VH and a VL of an anti-FX antibody selected from the group consisting of the anti-FX antibodies in FIG. 12A, FIG. 12B, and FIG. 12C.

Embodiment 124. The bispecific molecule of embodiment 122 or 123, which binds to the same epitope as a reference bispecific antibody, wherein the reference bispecific antibody comprises a VH and a VL of an anti-FIX antibody selected from the group consisting of the anti-FIX antibodies in FIG. 3A, FIG. 3B, FIG. 3C, and FIG. 3D and a VH and a VL of an anti-FX antibody selected from the group consisting of the anti-FX antibodies in FIG. 12A, FIG. 12B, and FIG. 12C.

Embodiment 125. The bispecific molecule of any one of embodiments 122 to 124, wherein (i) the anti-FIX antibody, or antigen binding portion thereof, comprises VH CDR1, CDR2, and CDR3 and VL CDR1, CDR2, and CDR3, wherein the VH CDR1, CDR2, and CDR3 and the VL CDR1, CDR2, and CDR3 are selected from the group consisting of VH CDR1s, VH CDR2s, and VH CDR3s and VL CDR1s, VL CDR2s, and VL CDR3s of the anti-FIX (BIIB-9) antibodies in FIG. 16A, FIG. 16B, FIG. 16C, and FIG. 16D; and (ii) the anti-FX antibody, or antigen binding portion thereof, comprises VH CDR1, CDR2, and CDR3 and VL CDR1, CDR2, and CDR3, wherein the VH CDR1, CDR2, and CDR3 and the VL CDR1, CDR2, and CDR3 are selected from the group consisting of VH CDR1s, VH CDR2s, and VH CDR3s and VL CDR1s, VL CDR2s, and VL CDR3s of the anti-FX (BIIB-12) antibodies in FIG. 16A, FIG. 16B, FIG. 16C, and FIG. 16D.

Embodiment 126. The bispecific molecule of any one of embodiments 122 to 124, wherein (a) the anti-FIX antibody, or antigen binding portion thereof, comprises:

(a1) VH CDR1, CDR2, and CDR3 sequences comprising SEQ ID NOs: 815, 860, or 905, respectively, and/or VL CDR1, VL CDR2, and VL CDR3 sequences comprising SEQ ID NOs: 950, 995, or 1040, respectively (BIIB-9-484);

(a2) VH CDR1, CDR2, and CDR3 sequences comprising SEQ ID NOs: 822, 867, and 912, respectively, and/or VL CDR1, CDR2, and CDR3 sequences comprising SEQ ID NOs: 957, 1002, and 1047, respectively (BIIB-9-619);

(a3) VH CDR1, CDR2, and CDR3 sequences comprising SEQ ID NOs: 1347, 1351, and 1355, respectively, and/or VL CDR1, CDR2, and CDR3 sequences comprising SEQ ID NOs: 1359, 1363, and 1367, respectively (BIIB-9-578);

(a4) VH CDR1, CDR2, and CDR3 sequences comprising SEQ ID NOs: 843, 888, and 933, respectively, and/or VL CDR1, CDR2, and CDR3 sequences comprising SEQ ID NOs: 978, 1023, and 1068, respectively (BIIB-9-1335); or,

(a5) VH CDR1, CDR2, and CDR3 sequences comprising SEQ ID NOs: 844, 889, and 934, respectively, and/or VL CDR1, CDR2, and CDR3 sequences comprising SEQ ID NOs: 979, 1024, and 1069, respectively (BIIB-9-1336); and,

(b) the anti-FX antibody, or antigen binding portion thereof, comprises:

(b1) VH CDR1, CDR2, and CDR3 sequences comprising SEQ ID NOs: 1393, 1483, and 1573, respectively, and/or VL CDR1, CDR2, and CDR3 sequences comprising SEQ ID NOs: 1663, 1753, and 1843, respectively (BIIB-12-915);

(b2) VH CDR1, CDR2, and CDR3 sequences comprising SEQ ID NOs: 1395, 1485, and 1575, respectively, and/or VL CDR1, CDR2, and CDR3 sequences comprising SEQ ID NOs: 1665, 1755, and 1845, respectively (BIIB-12-917);

(b3) VH CDR1, CDR2, and CDR3 sequences comprising SEQ ID NOs: 1911, 1915, and 1919, respectively, and/or VL CDR1, CDR2, and CDR3 sequences comprising SEQ ID NOs: 1923, 1927, and 1931, respectively (BIIB-12-925);

(b4) VH CDR1, CDR2, and CDR3 sequences comprising SEQ ID NOs: 1409, 1499, and 1589, respectively, and/or VL CDR1, CDR2, and CDR3 sequences comprising SEQ ID NOs: 1679, 1769, and 1859, respectively (BIIB-12-932); or,

(b5) VH CDR1, CDR2, and CDR3 sequences comprising SEQ ID NOs: 1433, 1523, and 1613, respectively, and/or VL CDR1, CDR2, and CDR3 sequences comprising SEQ ID NOs: 1703, 1793, and 1883, respectively (BIIB-12-1306).

Embodiment 127. The bispecific molecule of any one of embodiments 122 to 124, wherein (a) the anti-FIX antibody, or antigen binding portion thereof, comprises:

(a1) a VH and a VL comprising SEQ ID NOs: 31 and 221, respectively (BIIB-9-484);

(a2) a VH and a VL comprising SEQ ID NOs: 45 and 235, respectively (BIIB-9-619);

(a3) a VH and a VL comprising SEQ ID NOs: 185 and 371, respectively (BIIB-9-578);

(a4) a VH and a VL comprising SEQ ID NOs: 87 and 221, respectively (BIIB-9-1335); or

(a5) a VH and a VL comprising SEQ ID NOs: 89 and 221, respectively (BIIB-9-1336); and, (b) the anti-FX antibody, or antigen binding portion thereof, comprises:

(b1) a VH and a VL comprising SEQ ID NOs: 423 and 611, respectively (BIIB-12-915);

(b2) a VH and a VL comprising SEQ ID NOs: 427 and 615, respectively (BIIB-12-917);

(b3) a VH and a VL comprising SEQ ID NOs: 559 and 747, respectively (BIIB-12-925);

(b4) a VH and VL comprising SEQ ID NOs: 455 and 643, respectively (BIIB-12-932); or,

(b5) a VH and a VL comprising SEQ ID NOs: 503 and 691, respectively (BIIB-12-1306).

Embodiment 128. The bispecific molecule of any one of embodiments 122 to 127, (i) wherein the anti-FIX antibody, or antigen binding portion thereof, comprises a VH and a VL comprising SEQ ID NOs: 31 and 221, respectively; (BIIB-9-484) and the anti-FX antibody, or antigen binding portion thereof, comprises a VH and a VL comprising SEQ ID NOs: 423 and 611, respectively (BIIB-12-915); (ii) wherein the anti-FIX antibody, or antigen binding portion thereof, comprises a VH and a VL comprising SEQ ID NOs: 31 and 221, respectively; (BIIB-9-484) and the anti-FX antibody, or antigen binding portion thereof, comprises a VH and a VL comprising SEQ ID NOs: 427 and 615, respectively (BIIB-12-917); (iii) wherein the anti-FIX antibody, or antigen binding portion thereof, comprises a VH and a VL comprising SEQ ID NOs: 31 and 221, respectively; (BIIB-9-484) and the anti-FX antibody, or antigen binding portion thereof, comprises a VH and a VL comprising SEQ ID NOs: 559 and 747, respectively (BIIB-12-925); (iv) wherein the anti-FIX antibody, or antigen binding portion thereof, comprises a VH and a VL comprising SEQ ID NOs: 31 and 221, respectively; (BIIB-9-484) and the anti-FX antibody, or antigen binding portion thereof, comprises a VH and a VL comprising SEQ ID NOs: 455 and 643, respectively (BIIB-12-932); (v) wherein the anti-FIX antibody, or antigen binding portion thereof, comprises a VH and a VL comprising SEQ ID NOs: 185 and 371, respectively; (BIIB-9-578) and the anti-FX antibody, or antigen binding portion thereof, comprises a VH and a VL comprising SEQ ID NOs: 423 and 611, respectively (BIIB-12-915); (vi) wherein the anti-FIX antibody, or antigen binding portion thereof, comprises a VH and a VL comprising SEQ ID NOs: 185 and 371, respectively; (BIIB-9-578) and the anti-FX antibody, or antigen binding portion thereof, comprises a VH and a VL comprising SEQ ID NOs: 427 and 615, respectively (BIIB-12-917); (vii) wherein the anti-FIX antibody, or antigen binding portion thereof, comprises a VH and a VL comprising SEQ ID NOs: 45 and 235, respectively; (BIIB-9-619) and the anti-FX antibody, or antigen binding portion thereof, comprises a VH and a VL comprising SEQ ID NOs: 427 and 615, respectively (BIIB-12-917); or, (viii) wherein the anti-FIX antibody, or antigen binding portion thereof, comprises a VH and a VL comprising SEQ ID NOs: 45 and 235, respectively; (BIIB-9-619) and the anti-FX antibody, or antigen binding portion thereof, comprises a VH and a VL comprising SEQ ID NOs: 559 and 747, respectively (BIIB-12-925).

Embodiment 129. The bispecific molecule of any one of embodiments 122 to 128, which functionally mimics activated factor VIII (FVIIIa) cofactor in at least one FVIIIa activity assay.

Embodiment 130. The bispecific molecule of embodiment 129, wherein the FVIIIa activity assay is selected from a chromogenic FXa generation assay, a one-stage clotting assay, or the combination thereof.

Embodiment 131. The bispecific molecule of embodiment 129 or 130, wherein the FVIIIa activity achieves at least 10%, 20%, 30%, 35%, 40%, 45% 50%, 60%, 70%, 80%, 90%, 100%, 110%, 120%, 130%, 140%, 150%, 160%, 170%, 180%, 190%, or 200% of the activity otherwise achieved by FVIII in the same assay.

Embodiment 132. The bispecific molecule of any one of embodiments 122 to 131, wherein the bispecific molecule is capable of generating thrombin from prothrombin, fibrin from fibrinogen, and/or fibrin clot in vitro or in vivo.

Embodiment 133. The bispecific molecule according to any one of embodiments 122 through 132, wherein the bispecific molecule concurrently binds to both FIXa and FX, as determined by BLI.

Embodiment 134. The bispecific molecule of any one of embodiments 122 to 133, wherein the bispecific molecule is of the IgG isotype.

Embodiment 135. The bispecific molecule of embodiment 134, wherein the IgG isotype is of the IgG1 subclass.

Embodiment 136. The bispecific molecule of embodiment 134, wherein the IgG isotype is of the IgG4 subclass.

Embodiment 137. The bispecific molecule of any one of embodiments 122 to 136, wherein the bispecific molecule is of a bispecific IgG format and is selected from the group consisting of the antibodies in TABLE 2.

Embodiment 138. The bispecific molecule of embodiment 137, wherein the bispecific molecule is of a bispecific heterodimeric format.

Embodiment 139. The bispecific molecule of any one of embodiments 122 to 138, wherein the bispecific molecule comprises two different heavy chains and two different light chains.

Embodiment 140. The bispecific molecule according to any one of embodiments 122 through 138, wherein the bispecific molecule comprises two identical light chains and two different heavy chains.

Embodiment 141. The bispecific molecule of any one of embodiments 122 to 140, wherein the bispecific molecule is capable of controlling or reducing the incidence of bleeding episodes in a subject having hemophilia.

Embodiment 142. The bispecific molecule of any one of embodiments 122 to 140, wherein the bispecific molecule is capable of maintaining homeostasis or in a subject having hemophilia.

Embodiment 143. The bispecific molecule of any one of embodiments 122 to 140, wherein the bispecific molecule is capable of providing routine prophylaxis in a subject having hemophilia.

Embodiment 144. The bispecific molecule of any one of embodiments 122 to 143, wherein the subject has developed or is expected to develop neutralizing antibodies against Factor VIII.

Embodiment 145. An immunoconjugate comprising the bispecific molecule of any one of embodiments 122 to 144, linked to an agent.

Embodiment 146. A composition comprising the bispecific molecule of any one of embodiments 122 to 144 or the immunoconjugate, of embodiment 145, and a carrier.

Embodiment 147. A kit comprising the bispecific molecule of any one of embodiments 122 to 144 or the immunoconjugate of embodiment 145 and instructions for use.

Embodiment 148. A nucleic acid sequence encoding the bispecific molecule of any one of embodiments 122 to 144.

Embodiment 149. A vector comprising the nucleic acid according to embodiment 148.

Embodiment 150. A host cell comprising the vector of embodiment 149.

Embodiment 151. The host cell of embodiment 150, wherein the host cell is a prokaryotic cell, a eukaryotic cell, a protist cell, an animal cell, a plant cell, a fungal cell, a yeast cell, an Sf9 cell, a mammalian cell, an avian cell, an insect cell, a CHO cell, a HEK cell, or a COS cell.

Embodiment 152. A method of producing a bispecific molecule, comprising culturing the host cell of embodiment 150 under conditions that allow the expression of the bispecific molecule.

Embodiment 153. The method of producing the bispecific molecule of any one of embodiments 122 to 144, further comprising conditions that enhance heterodimerization.

Embodiment 154. A method of promoting FX activation in a subject in need thereof, comprising administering to the subject a therapeutically effective amount of the bispecific molecule of any one of embodiments 122 through 144, the immunoconjugate of embodiment 145, the composition of embodiment 146, the nucleic acid of embodiment 148, the vector of embodiment 149, or the host cell of embodiment 150.

Embodiment 155. A method of reducing the frequency or degree of a bleeding episode in a subject in need thereof, comprising administering to the subject an effective amount of the bispecific molecule of any one of embodiments 122 through 144, the immunoconjugate of embodiment 145, the composition of embodiment 146, the nucleic acid of embodiment 148, the vector of embodiment 149, or the host cell of embodiment 150.

Embodiment 156. The method of embodiment 155, wherein the subject has developed or has a tendency to develop an inhibitor against Factor VIII (“FVIII”).

Embodiment 157. The method of embodiment 156, wherein the inhibitor against FVIII is a neutralizing antibody against FVIII.

Embodiment 158. The method of any one of embodiments 155 to 157, wherein the bleeding episode is the result of hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, bleeding in the illiopsoas sheath, or any combinations thereof.

Embodiment 159. A method of treating a blood coagulation disorder in a subject in need thereof, comprising administering to the subject an effective amount of the bispecific molecule of any one of embodiments 122 through 144, the immunoconjugate of embodiment 145, the composition of embodiment 146, the nucleic acid of embodiment 148, the vector of embodiment 149, or the host cell of embodiment 150.

Embodiment 160. The method of embodiment 159, wherein the blood coagulation disorder is hemophilia A or hemophilia B.

Embodiment 161. The method of any one of embodiments 154 to 160, wherein the subject is a human subject.

Embodiment 162. The method of any one of embodiments 154 to 160, wherein the subject is undergoing or has undergone FVIII replacement therapy.

Embodiment 163. The method of any one of embodiments 154 through 162, wherein the bispecific molecule is administered in combination with a hemophilia therapy.

Embodiment 164. The method of embodiment 163, wherein the hemophilia therapy is a FVIII replacement therapy.

Embodiment 165. The method of embodiment 163 or 164, wherein the bispecific molecule is administered before, during or after administration of the hemophilia therapy.

Embodiment 166. The method of any one of embodiments 154 through 165, wherein the bispecific molecule is administered intravenously or subcutaneously.

Embodiment 167. The method of any one of embodiments 154 through 166, wherein administration of the bispecific molecule reduces the frequency of break-through bleeding episodes, spontaneous bleeding episodes, or acute bleeding.

Embodiment 168. The method of embodiment 167, wherein administration of the bispecific molecule reduces the annualized bleed rate by 5%, 10%, 20%, 30%, or 50%.

Embodiment 169. An anti-FIXa antibody, or antigen binding portion thereof, which binds to the same epitope as BIIB-9-1336.

Embodiment 170. An anti-FIXa antibody, or antigen binding portion thereof, which binds to an epitope overlapping BIIB-9-1336 epitope.

Embodiment 171. An anti-FIXa antibody, or antigen binding portion thereof, which binds to an epitope region comprising at least one amino acid located between chymotrypsinogen numbering positions (i) 91 and 101, (ii) 125 and 128, (iii) 165 and 179, or (iv) 232 and 241 in the sequence of the heavy chain of FIXa.

Embodiment 172. An anti-FIXa antibody, or antigen binding portion thereof, which binds to an epitope comprising at least one of chymotrypsinogen numbering amino acid residues H91, H92, N93, H101, D125, K126, E127, Y128, R165, Y177, N178, N179, S232, R233, Y234, V235, N236, W237, E240, and K241 of the sequence of the heavy chain of FIXa.

Embodiment 173. The anti-FIXa antibody, or antigen binding portion thereof, of embodiment 171 or 172, wherein the epitope comprises chymotrypsinogen numbering amino acid residues N93, R165, N178, and R233 of the sequence of the heavy chain of FIXa.

Embodiment 174. The anti-FIXa antibody, or antigen binding portion thereof, of embodiments 171 to 173 wherein the epitope comprises chymotrypsinogen numbering amino acid residues H91, H92, N93, H101, D125, K126, E127, Y128, R165, Y177, N178, N179, S232, R233, Y234, V235, N236, W237, E240, and K241 of the sequence of the heavy chain of FIXa.

Embodiment 175. The anti-FIXa antibody, or antigen binding portion thereof, of embodiments 171 to 174, wherein the epitope does not comprise at least one of chymotrypsinogen numbering amino acid residues N100, K132, Y137, R170, T172, F174, T175, H185, E202, and G205 of the sequence of the heavy chain of FIXa.

Embodiment 176. The anti-FIXa antibody, or antigen binding portion thereof, of embodiments 171 to 175, wherein the epitope does not comprise chymotrypsinogen numbering amino acid residues N100, K132, Y137, R170, T172, F174, T175, H185, E202, and G205 of the sequence of the heavy chain of FIXa.

Embodiment 177. The anti-FIXa antibody, or antigen binding portion thereof, of embodiments 171 to 176, which binds to at least one amino acid residue in the light chain of FIXa (SEQ ID NO:756).

Embodiment 178. The anti-FIXa antibody, or antigen binding portion thereof, of embodiment 177, wherein the amino acid residue in the light chain of FIXa (SEQ ID NO:756) is K100.

Embodiment 179. The anti-FIXa antibody, or antigen binding portion thereof, of any one of embodiments 169 to 178, wherein the epitope overlaps the binding site of FVIIIa to FIXa.

Embodiment 180. The anti-FIXa antibody, or antigen binding portion thereof, of any one of embodiments 169 to 179, which cross-competes with FVIIIa for binding to FIXa.

Embodiment 181. The anti-FIXa antibody, or antigen binding portion thereof, of any one of embodiments 169 to 180, wherein the antibody or antigen binding portion thereof blocks binding of FVIIIa to FIXa.

Embodiment 182. The anti-FIXa antibody, or antigen binding portion thereof, of any one of embodiments 1 to 12, which specifically binds to FIXa, comprising VH CDR1, CDR2, and CDR3 and VL CDR1, CDR2, and CDR3, wherein

(i) the VH CDR1 comprises a VH CDR1 selected from the group consisting of VH CDR1s in TABLE 7 or the VH CDR1 with one or two mutations; and/or,
(ii) the VH CDR2 comprises a VH CDR2 selected from the group consisting of VH CDR2s in TABLE 7 or the VH CDR2 with one or two mutations; and/or,
(iii) the VH CDR3 comprises a VH CDR3 selected from the group consisting of VH CDR3s in TABLE 7 or the VH CDR3 with one or two mutations; and/or,
(iv) the VL CDR1 comprises a VL CDR1 selected from the group consisting of VL CDR1s in TABLE 7 or the VL CDR1 with one or two mutations; and/or,
(v) the VL CDR2 comprises a VL CDR2 selected from the group consisting of VL CDR2s in TABLE 7 or the VL CDR2 with one or two mutations; and/or,
(vi) the VL CDR3 comprises a VL CDR3 selected from the group consisting of VL CDR3s in TABLE 7 or the VL CDR3 with one or two mutations.

Embodiment 183. The anti-FIXa antibody, or antigen binding portion thereof, of embodiment 182 which specifically binds to FIXa, comprising VH CDR1, CDR2, and CDR3 and VL CDR1, CDR2, and CDR3, wherein the VH CDR3 comprises the amino acid sequence ARDXIGGYAGYYGMDV (SEQ ID NO: 2196), wherein X₁ is L or V.

Embodiment 184. The isolated anti-FIXa antibody, or antigen binding portion thereof, of embodiment 183, wherein

(i) the VH CDR1 comprises the amino acid sequence FTFX₁SX₂X₃MX₄ (SEQ ID NO: 2194), wherein X₁ is S, G or E, X₂ is Y or F, X₃ is S, E, G, or D, and X₄ is N, V, A, or T; and/or
(ii) the VH CDR2 comprises the amino acid sequence X₅ISX₆X₇X₈X₉X₁₀IYYADSVKG (SEQ ID NO: 2195), wherein X₅ is S, A, Y, or G, X₆ is S or A, X₇ is S, A, or G, X₃ is S, G, or D, X₉ is S, T, or G, and X₁₀ is Y or T.

Embodiment 185. The isolated anti-FIXa antibody, or antigen binding portion thereof, of any one of embodiments 182 to 184, which comprises VL CDR1, CDR2 and CDR3, wherein VL CDR3 comprises the amino acid sequence QQYANFPYT (SEQ ID NO:2168).

Embodiment 186. The isolated anti-FIXa antibody, or antigen binding portion thereof, of embodiment 185, which comprises VL CDR1, CDR2 and CDR3, wherein

(i) VL CDR1 comprises the amino acid sequence QASQDIANYLN (SEQ ID NO:2116); and/or,
(ii) VL CDR2 comprises the amino acid sequence DASNLET (SEQ ID NO:2142).

Embodiment 187. The anti-FIXa antibody, or antigen binding portion thereof, of embodiment 182, which comprises VH CDR1, CDR2, and CDR3 comprising a VH CDR1 selected from SEQ ID NOs: 2038 to 2047, a VH CDR2 selected from SEQ ID NOs: 2064 to 2073, and a VH CDR3 selected from SEQ ID NOs: 2090 to 2099, and/or VL CDR1, CDR2, and CDR3 comprising a VL CDR1 selected from SEQ ID NOs: 2116 to 2125, a VL CDR2 selected from SEQ ID NOs: 2142 to 2151, and a VL CDR3 selected from SEQ ID NOs: 2168 to 2177.

Embodiment 188. The isolated anti-FIXa antibody, or antigen binding portion thereof, of embodiment 182, which specifically binds to FIXa, comprising VH CDR1, CDR2, and CDR3 and VL CDR1, CDR2, and CDR3, wherein the VH CDR3 comprises the amino acid sequence X₁RDVX₂GYAGX₃YGMDV (SEQ ID NO: 2198), wherein X₁ is A or V, X₂ is G or S, and X₃ is Y or F.

Embodiment 189. The isolated anti-FIXa antibody, or antigen binding portion thereof, of embodiment 188, wherein (i) the VH CDR1 comprises the amino acid sequence FTFGSYDMN (SEQ ID NO: 2048); and/or (ii) the VH CDR2 comprises the amino acid sequence SISX₁X₂X₃SYIX₄YAX₅SVKG (SEQ ID NO: 2197), wherein X₁ is S or D, X₂ is G or S, X₃ is E or A, X₄ is Y or A, and X₅ is E or D.

Embodiment 190. The isolated anti-FIXa antibody, or antigen binding portion thereof, of any one of embodiments 182, 188 or 189, which specifically binds to FIXa, comprising VH CDR1, CDR2, and CDR3 and VL CDR1, CDR2, and CDR3, wherein the VL CDR3 comprises the amino acid sequence X₁QYAX₂FPYT (SEQ ID NO: 2201), wherein X₁ is Q or S, and X₂ is N or R.

Embodiment 191. The isolated anti-FIXa antibody, or antigen binding portion thereof, of embodiment 190, which comprises VL CDR1, CDR2 and CDR3, wherein

(i) the VL CDR1 comprises the amino acid sequence X₁AX₂X₃X₄IX₅X₆YLN (SEQ ID NO: 2199), wherein X₁ is Q, G, or E, X₂ is S or N, X₃ is Q or E, X₄ is D or Y, X₅ is A or S, X₆ is N or D; and/or
(ii) the VL CDR2 comprises the amino acid sequence DAX₇NLX₈X₉ (SEQ ID NO: 2200), wherein X₇ is S or A, X₈ is E, H or Q, and X₉ is Tor Y.

Embodiment 192. The anti-FIXa antibody, or antigen binding portion thereof, of embodiment 182, which comprises VH CDR1, CDR2, and CDR3 comprising a VH CDR1 selected from SEQ ID NOs: 2048 to 2052, a VH CDR2 selected from SEQ ID NOs: 2074 to 2078, and a VH CDR3 selected from SEQ ID NOs: 2100 to 2104, and/or VL CDR1, CDR2, and CDR3 comprising a VL CDR1 selected from SEQ ID NOs: 2126 to 2130, a VL CDR2 selected from SEQ ID NOs: 2152 to 2156, and a VL CDR3 selected from SEQ ID NOs: 2178 to 2182.

Embodiment 193. The isolated anti-FIXa antibody, or antigen binding portion thereof, of embodiment 182, which specifically binds to FIXa, comprising VH CDR1, CDR2, and CDR3 and VL CDR1, CDR2, and CDR3, wherein the VH CDR3 comprises the amino acid sequence ARDGPX₁X₂X₃DYYMDV (SEQ ID NO: 2204), wherein X₁ is R or Q, X₂ is V, D, L or E, and X₃ is S or V.

Embodiment 194. The isolated anti-FIXa antibody, or antigen binding portion thereof, of embodiment 193, which comprises VH CDR1, CDR2 and CDR3, wherein

(i) the VH CDR1 comprises the amino acid sequence YTFX₁X₂YX₃MH (SEQ ID NO: 2202), wherein X₁ is T or H, X₂ is S, G, or H, and X₃ is Y or P; and/or
(ii) the VH CDR2 comprises the amino acid sequence X₄INPSX₈GX₆TX₇YAQKFQG (SEQ ID NO: 2203), wherein X₄ is I or S, X₅ is G or R, X₆ is S or R, and X₇ is S or E.

Embodiment 195. The isolated anti-FIXa antibody, or antigen binding portion thereof, of any one of embodiments 182, 193 or 194, which specifically binds to FIXa, comprising VH CDR1, CDR2, and CDR3 and VL CDR1, CDR2, and CDR3, wherein the VL CDR3 comprises the amino acid sequence QQRDNWPFT (SEQ ID NO:2116).

Embodiment 196. The isolated anti-FIXa antibody, or antigen binding portion thereof, of embodiment 195, which comprises VL CDR1, CDR2 and CDR3, wherein

(i) the VL CDR1 comprises the amino acid sequence RASQSVSSYLA (SEQ ID NO:2116); and/or,
(ii) the VL CDR2 comprises the amino acid sequence DASNRAT (SEQ ID NO:2116).

Embodiment 197. The anti-FIXa antibody, or antigen binding portion thereof, of embodiment 182, wherein the anti-FIXa antibody or antigen binding portion thereof comprises VH CDR1, CDR2, and CDR3 comprising a VH CDR1 selected from SEQ ID NOs: 2053 to 2057, a VH CDR2 selected from SEQ ID NOs: 2079 to 2083, and a VH CDR3 selected from SEQ ID NOs: 2105 to 2109, and/or VL CDR1, CDR2, and CDR3 comprising a VL CDR1 selected from SEQ ID NOs: 2131 to 2135, a VL CDR2 selected from SEQ ID NOs: 2157 to 2161, and a VL CDR3 selected from SEQ ID NOs: 2183 to 2187.

Embodiment 198. The isolated anti-FIXa antibody, or antigen binding portion thereof, of embodiment 182, which specifically binds to FIXa, comprising VH CDR1, CDR2, and CDR3 and VL CDR1, CDR2, and CDR3, wherein the VH CDR3 comprises the amino acid sequence ARDKYQDYSX₁DI (SEQ ID NO: 2207), wherein X₁ is F or V.

Embodiment 199. The isolated anti-FIXa antibody, or antigen binding portion thereof, of embodiment 198, which comprises VH CDR1, CDR2 and CDR3, wherein

(i) the VH CDR1 comprises the amino acid sequence GSIX₁SX₂X₃YX₄WX₅(SEQ ID NO: 2205), wherein X₁ is S or A, X₂ is S, T, G, or V, X₃ is S or A, X₄ is Y or A, and X₅ is G, V, N, or S; and/or
(ii) the VH CDR2 comprises the amino acid sequence X₆IX₇X₈X₉GX₁₀TX₁₁YNPSLKS (SEQ ID NO: 2206), wherein X₆ is S or Y, X₇ is S, Y, R, T or Q, X₈ is Y, G, P or A, X₉ is S or Q, X₁₀ is S or K, and X₁₁ is Y or Q.

Embodiment 200. The isolated anti-FIXa antibody of embodiment 182, 198 or 199, which comprises VL CDR1, CDR2 and CDR3, wherein the VL CDR3 comprises the amino acid sequence QQANFLPFT (SEQ ID NO:2188).

Embodiment 201. The isolated anti-FIXa antibody, or antigen binding portion thereof, of embodiment 200, which comprises VL CDR1, CDR2 and CDR3, wherein

(i) the VL CDR1 comprises the amino acid sequence RASQGIDSWLA (SEQ ID NO:2136); and/or,
(ii) the VL CDR2 comprises the amino acid sequence AASSLQS (SEQ ID NO:2162).

Embodiment 202. The anti-FIXa antibody, or antigen binding portion thereof, of embodiment 182, wherein the anti-FIXa antibody or antigen binding portion thereof comprises VH CDR1, CDR2, and CDR3 comprising a VH CDR1 selected from SEQ ID NOs: 2058 to 2063, a VH CDR2 selected from SEQ ID NOs: 2084 to 2089, and a VH CDR3 selected from SEQ ID NOs: 2110 to 2115, and/or VL CDR1, CDR2, and CDR3 comprising a VL CDR1 selected from SEQ ID NOs: 2136 to 2141, a VL CDR2 selected from SEQ ID NOs: 2162 to 2167, and a VL CDR3 selected from SEQ ID NOs: 2188 to 2193.

Embodiment 203. The anti-FIXa antibody, or antigen binding portion thereof, of any one of embodiments 182 to 202, comprising a VH and a VL, wherein

(i) the VH comprises an amino acid sequence which is at least about 80%, at least about 85%, at least about 90%, at least about 95%, at least about 96%, at least about 97%, at least about 98%, at least about 99%, or about 100% identical to the amino acid sequence selected from the group consisting of SEQ ID NOs: 1935, 1939, 1943, 1947, 1951, 1955, 1959, 1963, 1967, 1971, 1975, 1979, 1983, 1987, 1991, 1995, 1999, 2003, 2007, 2011, 2015, 2019, 2023, 2027, 2031, and 2035; and/or
(ii) the VL comprises an amino acid sequence which is at least about 80%, at least about 85%, at least about 90%, at least about 95%, at least about 96%, at least about 97%, at least about 98%, at least about 99%, or about 100% identical to the amino acid sequence selected from the group consisting of SEQ ID NOs: 1937, 1941, 1945, 1949, 1953, 1957, 1961, 1965, 1969, 1973, 1977, 1981, 1985, 1989, 1993, 1997, 2001, 2005, 2009, 2013, 2017, 2021, 2025, 2029, 2033, and 2037.

Embodiment 204. The anti-FIXa antibody, or antigen binding portion thereof, of any one of embodiments 182 to 203, comprising a VH and a VL, wherein

(a1) the VH and the VL comprise SEQ ID NOs: 1935 and 1937, respectively (BIIB-9-3595);
(a2) the VH and the VL comprise SEQ ID NOs: 1939 and 1941, respectively (BIIB-9-3601);
(a3) the VH and the VL comprise SEQ ID NOs: 1943 and 1945, respectively (BIIB-9-3604);
(a4) the VH and the VL comprise SEQ ID NOs: 1947 and 1949, respectively (BIIB-9-3617);
(a5) the VH and the VL comprise SEQ ID NOs: 1951 and 1953, respectively (BIIB-9-3618);
(a6) the VH and the VL comprise SEQ ID NOs: 1955 and 1957, respectively (BIIB-9-3621);
(a7) the VH and the VL comprise SEQ ID NOs: 1959 and 1961, respectively (BIIB-9-3647);
(a8) the VH and the VL comprise SEQ ID NOs: 1963 and 1965, respectively (BIIB-9-3649);
(a9) the VH and the VL comprise SEQ ID NOs: 1967 and 1969, respectively (BIIB-9-3650);
(a10) the VH and the VL comprise SEQ ID NOs: 1971 and 1973, respectively (BIIB-9-3654);
(a11) the VH and the VL comprise SEQ ID NOs: 1975 and 1977, respectively (BIIB-9-3753);
(a12) the VH and the VL comprise SEQ ID NOs: 1979 and 1981, respectively (BIIB-9-3754);
(a13) the VH and the VL comprise SEQ ID NOs: 1983 and 1985, respectively (BIIB-9-3756);
(a14) the VH and the VL comprise SEQ ID NOs: 1987 and 1989, respectively (BIIB-9-3764);
(a15) the VH and the VL comprise SEQ ID NOs: 1991 and 1993, respectively (BIIB-9-3766);
(a16) the VH and the VL comprise SEQ ID NOs: 1995 and 1997, respectively (BIIB-9-3707);
(a17) the VH and the VL comprise SEQ ID NOs: 1999 and 2001, respectively (BIIB-9-3709);
(a18) the VH and the VL comprise SEQ ID NOs: 2003 and 2005, respectively (BIIB-9-3720);
(a19) the VH and the VL comprise SEQ ID NOs: 2007 and 2009, respectively (BIIB-9-3727);
(a20) the VH and the VL comprise SEQ ID NOs: 2011 and 2013, respectively (BIIB-9-3745);
(a21) the VH and the VL comprise SEQ ID NOs: 2015 and 2017, respectively (BIIB-9-3780);
(a22) the VH and the VL comprise SEQ ID NOs: 2019 and 2021, respectively (BIIB-9-3675);
(a23) the VH and the VL comprise SEQ ID NOs: 2023 and 2025, respectively (BIIB-9-3681);
(a24) the VH and the VL comprise SEQ ID NOs: 2027 and 2029, respectively (BIIB-9-3684);
(a25) the VH and the VL comprise SEQ ID NOs: 2031 and 2033, respectively (BIIB-9-3698); or,
(a26) the VH and the VL comprise SEQ ID NOs: 2035 and 2037, respectively (BIIB-9-3704).

### BRIEF DESCRIPTION OF THE DRAWINGS/FIGURES

FIG. 1A is a schematic representation of the domain organization of FIX zymogen and activated FIX with and without a substrate mimic bound in the active site (FIXa+EGR-CMK) (free FIXa and FIXa-SM, respectively). FIG. 1B is a schematic representation of the domain organization of FX zymogen and activated FX with and without a substrate mimic bound in the active site (FXa+EGR-CMK). In this representation, HC is the heavy chain of FIX, FIXa, FX, or FXa. LC is the light chain of FIX, FIXa, FX, or FXa.

FIG. 2 shows a schematic representation of the antibody generation, antibody characterization, and functional characterization for certain embodiments described herein.

FIGS. 3A-3D depict 95 anti-FIX antibodies disclosed herein, discovered by the antibody generation process described in FIG. 2. The germline, CDR length, CDR amino acid sequences, and SEQ ID NO are provided for both the VH and VL the antibodies. The full sequences of the VH and VL are provided in TABLE 4. The SEQ ID number for each CDR sequence is represented in TABLE 4. FIGS. 3A-3C present antibodies that preferentially bind to activated clotting factor IX (FIXa) (e.g., free FIXa and/or FIXa covalently modified by EGR- or LTR-CMK (FIXa-SM) compared to FIX zymogen (e.g., non-activatable FIX). In particular, FIG. 3A lists antibodies preferentially bind to FIXa-SM compared to free FIXa or FIXa zymogen (e.g., non-activatable FIX) (Class I). FIG. 3B lists antibodies preferentially bind to free FIXa compared to FIXa-SM or FIX zymogen (e.g., non-activatable FIX) (Class II). FIG. 3C lists antibodies that bind to either FIXa-SM or free FIXa, but do not appreciably bind to FIX zymogen (e.g., non-activatable FIX) (Class III). FIG. 3D lists antibodies that preferentially bind to FIX zymogen (e.g., non-activatable FIX) compared to free FIXa or FIXa-SM) (Class IV).

FIGS. 4A and 4B shows binding by Bio-Layer Interferometry (BLI) measurements of sensor-associated IgG to the indicated antigen, FIX zymogen (e.g., non-activatable FIX) (Haematologic Technologies, Inc., Essex Junction, Vt., USA) or FIXa (Haematologic Technologies, Inc., Essex Junction, Vt., USA). The maximum BLI response (nm) for each antibody is plotted on the y-axis.

FIG. 5 is a table presenting apparent monovalent affinity values (K_{D}) to free FIXa for each of the listed antibodies as determined by 1:1 fitting algorithms provided in the ForteBio Data Analysis 9.0 software.

FIGS. 6A-6E depict the measurement of binding by BLI of sensor associated IgG to the indicated antigen (free FIXa or FIX zymogen (e.g., non-activatable FIX)). FIG. 6A shows the measurement of binding for antibody BIIB-9-484 (VH: SEQ ID NO:31; VL: SEQ ID NO:221). FIG. 6B shows the measurement of binding of antibody BIIB-9-440 (VH: SEQ ID NO:19; VL: SEQ ID NO:209). FIG. 6C shows the measurement of binding of antibody BIIB-9-882 (VH: SEQ ID NO:115; VL: SEQ ID NO:301). FIG. 6D shows the measurement of binding of antibody BIIB-9-460 (VH: SEQ ID NO:23; VL: SEQ ID NO:213). FIG. 6E shows the measurement of binding for antibody BIIB-9-433 (VH: SEQ ID NO: 127; VL: SEQ ID NO: 313). The plots show the BLI response (nm) for association and dissociation as a function of time. FIG. 6F depicts a table of apparent monovalent affinity (K_{D}) to FIX zymogen (e.g., non-activatable FIX) and free FIXa, respectively, for each of the listed antibodies (i.e., BIIB-9-484, BIIB-9-440, BIIB-9-882, BIIB-9-460, and BIIB-9-433) described in FIGS. 6A-6E, as determined by 1:1 fitting algorithms provided in the ForteBio Data Analysis 9.0 software.

FIG. 7 shows the measurement of binding by BLI of sensor-associated IgG to the indicated antigen, free FIXa (Haematologic Technologies, Inc., Essex Junction, Vt., USA) or FIXa-SM (e.g., FIXa+EGR-CMK (Haematologic Technologies, Inc., Essex Junction, Vt., USA)). The maximum BLI response (nm) for each antibody is plotted on the y-axis.

FIGS. 8A-8E exhibit the measurement of binding by BLI of sensor associated IgG to the indicated antigens (FIXa-SM, e.g., FIXa+EGF-CMK, and free FIXa). FIG. 8A shows the measurement of binding for antibody BIIB-9-484 (VH: SEQ ID NO:31; VL: SEQ ID NO:221). FIG. 8B shows the measurement of binding of antibody BIIB-9-440 (VH: SEQ ID NO:19; VL: SEQ ID NO:209). FIG. 8C shows the measurement of binding of antibody BIIB-9-882 (VH: SEQ ID NO:115; VL: SEQ ID NO:301). FIG. 8D shows the measurement of binding of antibody BIIB-9-460 (VH: SEQ ID NO:23; VL: SEQ ID NO:213). FIG. 8E shows the measurement of binding for antibody BIIB-9-433 (VH: SEQ ID NO: 127; VL: SEQ ID NO: 313). The provided plots show the BLI response (nm) for association and dissociation as a function of time. FIG. 8F depicts a table of apparent monovalent affinity (K_{D}) to (i) free FIXa or (ii) FIXa-SM (e.g., FIXa+EGR-CMK) for each of the listed antibodies (i.e., BIIB-9-484, BIIB-9-440, BIIB-9-882, BIIB-9-460, and BIIB-9-433) as determined by 1:1 fitting algorithms provided in the ForteBio Data Analysis 9.0 software.

FIGS. 9A-9D display the measurement of binding by BLI of listed sensor-associated IgG to the indicated antigen FIXn (non-activatable FIX), free FIXa, or FXa-SM (e.g., FIXa+EGR-CMK) (all obtained from Haematologic Technologies, Inc., Essex Junction, Vt., USA) as a function of time. The maximum BLI response (nm) is provided on each sensorgram. FIG. 9A shows the measurement of binding for representative antibodies in Class I (FIG. 3A), e.g., antibody BIIB-9-484 and antibody BIIB-9-460 (VH: SEQ ID NO: 23; VL: SEQ ID NO:213). FIG. 9B shows the measurement of binding for representative antibodies in Class II (FIG. 3B), e.g., antibody BIIB-9-416 (VH: SEQ ID NO:93; VL: SEQ ID NO:279) and antibody BIIB-9-885 (VH: SEQ ID NO:97; VL: SEQ ID NO:283). FIG. 9C shows the measurement of binding for a representative antibody in Class III (FIG. 3C), e.g., antibody BIIB-9-1287 (VH: SEQ ID NO:181; VL: SEQ ID NO:367). FIG. 9D shows the measurement of binding for a representative antibody in Class IV (FIG. 3D), e.g., antibody BIIB-9-397 (VH: SEQ ID NO:183; VL: SEQ ID NO:369).

FIG. 10 shows a table listing 95 antibodies disclosed herein, in addition to their assigned class based on antigen binding profiles as determined by BLI binding assays. Antibodies were assigned to classes if they exhibited a 0.1 or greater difference in BLI response to one antigen over another using the assay parameters defined in the examples. Class I antibodies corresponds to the antibodies in FIG. 3A. Class II antibodies correspond to the antibodies in FIG. 3B. Class III antibodies correspond to the antibodies in FIG. 3C. Class IV antibodies correspond to the antibodies in FIG. 3D.

FIG. 11 shows a table listing the maximum wavelength (nm) of each indicated antibody as determined by screening for propensity to self-interact by AC-SINS. A threshold value of 540 nm is set based on internal controls, with antibodies exceeding the threshold shaded in black (indicating a potential for self-interaction).

FIGS. 12A-12C are tables listing 94 antibodies disclosed herein, discovered by the antibody generation process. The germline, CDR length, CDR amino acid sequences, and SEQ ID NOs are provided for both the VH and VL of each of the 94 antibodies. The sequences of the complete VH and VL for each antibody are presented in TABLE 4. FIG. 12A and FIG. 12B present antibodies that preferentially bind to FX zymogen (e.g., non-activatable FX) compared to activated clotting factor FX (FXa) (e.g., FXa covalently modified by EGR- or LTR-CMK (FXa-SM) (Class V). FIG. 12C presents antibodies that preferentially bind to activated clotting factor FX (FXa) (e.g., FXa covalently modified by EGR- or LTR-CMK (FXa-SM) compared to FX zymogen (e.g., non-activatable FX) (Class VI).

FIGS. 13A and 13B depict the measurement of binding by BLI of sensor-associated IgG to the indicated antigen, FX zymogen or FXa-SM (e.g., FXa+EGR-CMK). The maximum BLI response (nm) for each antibody is plotted on the y-axis.

FIG. 14 shows a table of apparent monovalent affinity (K_{D}) to FX zymogen in M for each of the listed antibodies as determined by 1:1 fitting algorithms provided in the ForteBio Data Analysis 9.0 software.

FIG. 15 shows a table listing the maximum wavelength (nm) of each indicated antibody as determined by screening for propensity to self-interact by AC-SINS. A threshold value of 540 nm was set based on internal controls. Antibodies exceeding the threshold are shaded in black, which indicates a potential for self-interaction.

FIGS. 16A-16D show 202 bispecific antibodies identified as having the ability to replace FVIIIa-like function in a chromogenic Factor Xa generation assay. The 202 bispecific antibodies are separated into four groups, and four corresponding sub-figures. FIG. 16A shows the rates of FXa chromogenic substrate cleavage for the first group of bispecific antibodies (1-51 of 202). FIG. 16B shows the rates of FXa chromogenic substrate cleavage for the second group of bispecific antibodies (52-102 of 202). FIG. 16C shows the rates of FXa chromogenic substrate cleavage for the third group of bispecific antibodies (103-152 of 202). FIG. 16D shows the rates of FXa chromogenic substrate cleavage for the fourth group of bispecific antibodies (153-202 of 202). In each case, the mean baseline rate in the absence of bispecific antibody is indicated by the dashed line.

FIG. 17 depicts the rates of FXa chromogenic substrate cleavage for a subset of bispecific antibodies in the IgG4 format. The mean baseline rate in the absence of bispecific antibody is indicated by the dashed line.

FIG. 18 illustrates the kinetics of FXa chromogenic substrate cleavage in the presence of three representative bispecific antibodies. Compared to the baseline control (in which no antibody was added to the reaction mixture), BIIB-9-484/BIIIB-12-915, BIIB-9-619/BIIB-12-925, and BIIB-9-578/BIIB-12-917 were able to increase the rate of FXa chromogenic substrate cleavage, as indicated by the increase in OD over time.

FIG. 19 illustrates the ability of BIIB-9-484/BIIB-12-917, BIIB-9-484/BIIB-12-915, and BIIB-9-484/BIIB-12-1306 to replace the function of FVIIIa in a one-stage clotting assay in FVIII deficient plasma (as indicated by the decrease in clotting time). FVIII-deficient plasma with no bispecific antibody is shown for comparison.

FIG. 20 shows that the ability of a bispecific antibody, BIIB-9-484/BIIB-12-917, to replace FVIIIa-function in a one-stage clotting assay in FVIII deficient plasma is dependent on the bispecific format. Homodimeric BIIB-9-484, homodimeric BIIB-12-917, and a mixture of the two homodimers were unable to replace FVIIIa-like function. FVIII deficient plasma with no antibody is shown for comparison.

FIG. 21 exhibits the BLI-binding assay to assess co-binding of each target antigen to the indicated bispecific antibody using dip and read streptavidin biosensors. The sensorgram plots the BLI response (nm) as a function of time, with each stage of the experiment separated by a black vertical line. An increase in response at a particular stage is indicative of protein loading.

FIG. 22A lists the CDR sequences of BIIB-9-484 and two affinity matured daughters, BIIB-9-1335 and BIIB-9-1336. A CLUSTAL format multiple sequence alignment by MAFFT (v7.205) of the VH segments of BIIB-9-484, BIIB-9-1335, BIIB-9-1336 is provided. Degree of amino acid conservation is indicating above the alignment (“*”=identical; “:”=strongly conserved; “.”=poorly conserved), as well as the bars below the alignment. The VH and VL CDRs are underlined. The sequence before VH-CDR1 is framework region (FR) 1; the sequence after VH-CDR1 and before VH-CDR2 is FR2; the sequence after VH-CDR2 and before VH-CDR3 is FR3; and the sequence after VH-CDR3 is FR4. The sequence before VL-CDR1 is framework region (FR) 1; the sequence after VL-CDR1 and before VL-CDR2 is FR2; the sequence after VL-CDR2 and before VL-CDR3 is FR3; and the sequence after VL-CDR3 is FR4. FIGS. 22B-22D show the BLI-binding profile of free FIXa to BIIB-9-484, BIIB-9-1335, and BIIB-9-1336, respectively.

FIG. 23 shows that increasing the affinity of the anti-FIXa arm in the context of a bispecific antibody with FVIIIa-like activity results in higher activity in a one-stage clotting assay. An example of this is illustrated in the plot above, in which bispecific antibodies with high affinity anti-FIXa arms (BIIB-9-1335/BIIB-12-917 and BIIB-9-1336/BIIB-12-917) show a further decrease in clotting time compared to the bispecific antibody with a lower affinity anti-FIXa arm (BIIB-9-484/BIIB-12-917).

FIG. 24A shows the binding affinity of emicizumab (ACE910) sequence identical bispecific antibody (“emicizumab biosimilar”) to FIX zymogen, activated FIX, FX zymogen, and activated FX (i.e., 1 μM, 1 μM, 1 μM, and 1 μM, respectively). FIG. 24B shows the binding affinity of a bispecific molecule (BS-027125) to FIX zymogen, activated FIX, FX zymogen, and activated FX (i.e., 8 nM, 2 nM, 20 nM, and not detected, respectively).

FIG. 25 shows the clotting time (sec) of FVIII (inverted triangle), BS-025 FIXa homodimer (big diamond), BS-027 FX homodimer (small diamond), BS-027025 homodimer mixture (triangle), and BS-027025 bispecific (square) over various concentrations (IU/mL or μM).

FIG. 26 shows the FVIII activity (FVIII equivalent %) by BS-027125 (Square), BS-027125 FIXa homodimer (triangle), and BS-027125 FX homodimer (circle). The FVIII activity was measured by one-stage clotting assay.

FIG. 27 shows the nM FXa generation by rFVIII (big circle), BS-027125 (square), BS-027125 FIXa homodimer (triangle), BS-027125 FX homodimer (inverted triangle), and BS-027125 (no PL) (small circle) measured by chromogenic factor Xa (FXa) generation assay.

FIG. 28A shows the results of thrombin generation assay of BS-027125. The left panel shows the lag time (min), and the right panel shows the peak.

FIG. 28B shows the amount of thrombin generated by rFVIII (left panel) and BS-027125 (right panel).

FIG. 29 shows nM FXa generated in the presence of rFVIII, emicizumab biosimilar, or BS-027125 with either PC/PS (80%/20%) or PC/PEPS (40%/40%/20%) phospholipid vesicles (upper panels) and the fold change in activity contributed by PC/PEPS over PC/PS phospholipid vesicles (lower panels).

FIG. 30 shows the results of thrombin generation assay on PC/PEPS phospholipids in the presence of rFVIII, emicizumab biosimilar, and BS-027125. The upper panels show the lag time (min); the middle panels show the peak thrombin (nM); and the lower panels show the endogenous thrombin potential (ETP) (nM*min).

FIGS. 31A, 31B, 31C and 31D show the epitope binning of 47 different anti-FIXa antibodies against each other as determined by biolayer interferometry. FIG. 31A and FIG. 31B show the Octet profiles for non-competitive and competitive binders, respectively. FIG. 31C summarizes the 47×47 interactions tested and whether the antibodies pairs result in competitive or non-competitive binding. Dark gray squares indicate antibody pairs that cross-block, indicating that they fall into the same bin. Medium gray squares indicate antibody pairs that do not cross-block, indicating that they fall into different bins. White squares indicate unidirectional conflicts and light gray squares indicate antibodies for which data could not be analyzed. FIG. 31D shows node analysis of the binning network determined in FIG. 31C. The closer together two antibodies are on this map, the more similar their binning profiles are.

FIG. 32A shows the binding profile of BIIB-9-484 to FIXa in the presence and absence of calcium using biolayer interferometry. The dashed line indicates the end of the association phase and the start of the dissociation phase.

FIG. 32B shows the binding profile of BIIB-9-1336 to FIXa in the presence and absence of calcium using biolayer interferometry. The dashed line indicates the end of the association phase and the start of the dissociation phase.

FIG. 33A shows the rate of substrate cleavage by FIXa (250 nM) alone or in the presence of increasing concentrations of two different anti-FIXa antibodies (BIIB-9-1336 and BIIB-9-579) or an anti-FX antibody control (BIIB-12-917). BIIB-9-1336 was tested as a homodimeric, bivalent antibody (“BIIB-9-1336’), as a one armed antibody (“BIIB-9-1336 one-armed”) and in a bispecific configuration with BIIB-12-917 (“BIIB-9-1336/BIIB-12-917”). The rate of substrate cleavage is expressed in mOD/minute and increasing amounts of antibody are expressed in terms of the concentration (nM) of FIXa-Ab complex.

FIG. 33B shows the fold increase in FIXa amidolytic activity that is seen for 500 nM FIXa in the presence of a panel of BIIB-9-484, BIIB-9-1336, BIIB-9-619, and BIIB-9-578 antibodies.

FIG. 33C shows the rate of substrate cleavage by FIXa across varying concentrations of substrate in the presence or absence of saturating amounts of BIIB-9-1336.

FIG. 33D shows the K_{M} and Vₘₐₓ for FIXa in the presence and absence of BIIB-9-1336.

FIG. 34A and FIG. 34B show the rate of ATIII inhibition of FIXa in the presence or absence of anti-FIXa antibodies. FIG. 34A shows the appearance of a band at 75 kDa corresponding to ATIII-FIXa complex. The rate of complex formation is indicative of ATIII inhibition of FIXa and is increased in the presence of BIIB-9-1335 and BIIB-9-1336. FIG. 34B shows the band intensity of ATIII-FIXa complex formation quantified and graphed at different time points.

FIG. 35 is cartoon representation of the crystal structure of the Fab region of BIIB-9-1336 in complex with the EGF2 and serine protease domain of FIXa shown in two orientations.

FIG. 36 shows the serine protease domain of FIXa in surface representation with the epitopes of BIIB-9-1336 (1336 epitope) and FVIIIa (FVIIIa epitope) mapped onto the surface. The BIIB-9-1336 epitope on FIXa and the FVIIIa epitope on FIXa are colored in black. Residues shared between the two epitopes are outlined in white.

FIG. 37 lists the specific amino acids residues in the FIXa heavy chain that constitute the BIIB-9-1336 and FVIIIa epitopes and correspond to the residues highlighted in FIG. 36. Residues shared between the two epitopes are shown underlined and in bold. Residues shown in parentheses are not shown in FIG. 36 as they do not agree across published reports. The numbering of the amino acid residues are based on the chymotrypsinogen numbering. BIIB-9-1336 also contacts one residue in the light chain of FIXa and is indicated by the asterisk. Light chain contacts for FVIIIa are not listed.

FIG. 38A shows the binding of BIIB-12-917 to a panel of FX variants including wild type Factor X zymogen, wild type activated FX, activated FX that retains the activation peptide, zymogen FX lacking the activation peptide, and a chimeric FIX construct in which the FIX activation peptide has been replaced by the FX activation peptide, by biolayer interferometry. The dashed line indicates the end of the association phase and the start of the dissociation phase.

FIG. 38B shows cartoon representations of each of the above-mentioned FX variants. The + and − signs indicate whether BIIB-12-917 binds. These data show that the epitope of BIIB-12-917 is in the activation peptide region of FX.

### DETAILED DESCRIPTION OF THE INVENTION

The present disclosure provides antibodies that preferentially bind to specific forms of clotting factors. In particular, the disclosure provides antibodies and antigen binding portion thereof that specifically binds to FIX (e.g., antibodies and antigen binding portion thereof that preferentially bind to activated factor IX (FIXa) in the presence of FIXa and factor IX zymogen (FIXz)). The disclosure also provides antibodies and antigen binding portions thereof that specifically and preferentially bind to FX (FX zymogen (FXz)) in the presence of FXz and FXa.

Also provided are bispecific molecules (e.g., antibodies) comprising any one of anti-FIX antibodies or antigen binding portions thereof disclosed herein and any one of anti-FX antibodies or antigen binding portions thereof disclosed herein. These bispecific antibodies can bind simultaneously to FIX and FX and mimic the function of coagulation factor Villa.

The present disclosure also provides, for example, compositions comprising the binding molecules, e.g., antibodies, disclosed herein (e.g., pharmaceutical or diagnostic compositions), nucleic acids and vectors encoding the binding molecules disclosed herein, cells comprising nucleic acids encoding the binding molecules disclosed herein, methods of making, methods of treatment and diagnosis, immunoconjugates, and kits.

The headings provided herein are not limitations of the various aspects or aspects of the disclosure, which can be defined by reference to the specification as a whole. Accordingly, the terms defined immediately below are more fully defined by reference to the specification in its entirety. Before describing the present invention in detail, it is to be understood that this invention is not limited to specific compositions or process steps, as such can vary.

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

The invention includes embodiments in which exactly one member of the group is present in, employed in, or otherwise relevant to a given product or process. The invention includes embodiments in which more than one, or all of the group members are present in, employed in, or otherwise relevant to a given product or process.

In this specification and the appended claims, the singular forms “a”, “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Wherever aspects are described herein with the language “comprising,” otherwise analogous aspects described in terms of “consisting of” and/or “consisting essentially of” are also provided.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the invention. Where a value is explicitly recited, it is to be understood that values which are about the same quantity or amount as the recited value are also within the scope of the invention. Where a combination is disclosed, each subcombination of the elements of that combination is also specifically disclosed and is within the scope of the invention. Conversely, where different elements or groups of elements are individually disclosed, combinations thereof are also disclosed. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

Nucleotides are referred to by their commonly accepted single-letter codes. Unless otherwise indicated, nucleic acids are written left to right in 5′ to 3′ orientation. Nucleotides are referred to herein by their commonly known one-letter symbols recommended by the IUPAC-IUB Biochemical Nomenclature Commission. Accordingly, A represents adenine, C represents cytosine, G represents guanine, T represents thymine, U represents uracil.

Amino acids are referred to herein by either their commonly known three letter symbols or by the one-letter symbols recommended by the IUPAC-IUB Biochemical Nomenclature Commission. Unless otherwise indicated, amino acid sequences are written left to right in amino to carboxy orientation.

About: The term “about” as used in connection with a numerical value throughout the specification and the claims denotes an interval of accuracy, familiar and acceptable to a person skilled in the art. In general, such interval of accuracy is f 10%.

Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

Administered in combination: As used herein, the term “administered in combination,” “combined administration,” or “combination therapy” means that two or more agents, e.g., a binding molecule disclosed herein, and a second agent, are administered to a subject at the same time or within an interval such that there can be an overlap of an effect of each agent on the patient. In some embodiments, they are administered within about 60, 30, 15, 10, 5, or 1 minute of one another. In some embodiments, the administrations of the agents are spaced sufficiently closely together such that a combinatorial (e.g., a synergistic) effect is achieved.

Affinity: The term “affinity” refers to the degree to which a binding molecule, e.g., an antibody, binds to an antigen so as to shift the equilibrium of antigen and binding molecule toward the presence of a complex formed by their binding. Thus, where an antigen and binding molecule are combined in relatively equal concentration, a binding molecule of high affinity will bind to the available antigen so as to shift the equilibrium toward high concentration of the resulting complex. Binding molecules, e.g., antibodies, or antigen-binding fragments, variants or derivatives thereof of the present disclosure can also be described or specified in terms of their binding affinity to an antigen. The affinity of binding molecule, e.g., an antibody, for an antigen can be determined experimentally using any suitable method. (See, e.g., Berzofsky et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein).

The measured affinity of a particular binding molecule-antigen interaction can vary if measured under different conditions (e.g., salt concentration, pH). Thus, measurements of affinity and other antigen-binding parameters (e.g., K_{D}, Kₐ, K_{d}) are preferably made with standardized solutions of binding molecule and antigen, and a standardized buffer.

The “high affinity” for a binding molecule, e.g., an antibody, refers to an equilibrium association constant (K_{aff}) of at least about 1×10⁷ liters/mole, or at least about 1×10⁸ liters/mole, or at least about 1×10⁹ liters/mole, or at least about 1×10¹⁰ liters/mole, or at least about 1×10¹¹ liters/mole. or at least about 1×10¹² liters/mole, or at least about 1×10¹³ liters/mole, or at least about 1×10¹⁴ liters/mole or greater. “High affinity” binding can vary for antibody isotypes.

K_{D}, the equilibrium dissociation constant, is a term that is also used to describe antibody affinity and is the inverse of Kn. K_{D} is obtained from the ratio of k_{d} to kₐ (i.e., k_{d}/kₐ) and is expressed as a molar concentration (M). K_{D} values for antibodies can be determined using methods well established in the art. Available methods for determining the K_{D} of an antibody include a Bio-Layer Interferometry (BLI) assay, surface plasmon resonance, a biosensor system such as a BIACORE® system or flow cytometry and Scatchard analysis. If K_{D} is used, the term “high affinity” for an antibody refers to an equilibrium dissociation constant (K_{D}) of less than about 1×10⁻⁷ M, or less than about 1×10⁻⁸ M, or less than about 1×10⁻⁹ M, or less than about 1×10⁻¹⁰ M, or less than about 1×10⁻¹¹ M, or less than about 1×10⁻¹² M, or less than about 1×10¹³ M, less than about 1×10⁻¹⁴ M, or lower.

Amino acid substitution: The term “amino acid substitution” refers to replacing an amino acid residue present in a parent or reference sequence (e.g., a wild type sequence) with another amino acid residue. An amino acid can be substituted in a parent or reference sequence (e.g., a wild type polypeptide sequence), for example, via chemical peptide synthesis or through recombinant methods known in the art. Accordingly, a reference to a “substitution at position X” refers to the substitution of an amino acid present at position X with an alternative amino acid residue. In some aspects, substitution patterns can be described according to the schema AnY, wherein A is the single letter code corresponding to the amino acid naturally or originally present at position n, and Y is the substituting amino acid residue. In other aspects, substitution patterns can be described according to the schema An(YZ), wherein A is the single letter code corresponding to the amino acid residue substituting the amino acid naturally or originally present at position n, and Y and Z are alternative substituting amino acid residues that can replace A

In the context of the present disclosure, substitutions (even when they are referred to as amino acid substitution) are conducted at the nucleic acid level, i.e., substituting an amino acid residue with an alternative amino acid residue is conducted by substituting the codon encoding the first amino acid with a codon encoding the second amino acid.

Affinity matured: The term “affinity matured” refers to a binding molecule, e.g., an antibody, that has undergone affinity maturation, a process by which binding molecules, e.g., antibodies, with increased affinity for a target antigen are produced. Thus, an affinity matured antibody is an antibody with one or more alterations in one or more CDRs thereof which result an improvement in the affinity of the antibody for antigen, compared to a parent antibody which does not possess those alteration(s). Exemplary affinity matured antibodies will have nanomolar or even picomolar affinities for the target antigen.

Any one or more methods of preparing and/or using affinity maturation libraries available in the art may be employed in order to generate affinity matured antibodies in accordance with various embodiments of the invention disclosed herein. Exemplary affinity maturation methods include random mutagenesis, bacterial mutator strains passaging, site-directed mutagenesis, mutational hotspots targeting, parsimonious mutagenesis, antibody shuffling, light chain shuffling, heavy chain shuffling, CDR1 and/or CDR1 mutagenesis, and methods of producing and using affinity maturation libraries amenable to implementing methods and uses in accordance with various embodiments of the invention disclosed herein, include, for example, those disclosed in: Prassler et al. (2009); Immunotherapy, Vol. 1 (4), pp. 571-583; Sheedy et al. (2007), Biotechnol. Adv., Vol. 25(4), pp. 333-352; WO2012/009568; WO2009/036379; WO2010/105256; US2002/0177170; WO2003/074679, all of which are herein incorporated by reference in their entirety. Marks et al. (1992) BioTechnology 10:779-783 describes affinity maturation by VH and VL domain shuffling. Random mutagenesis of CDR and/or framework residues is described by Barbas et al. (1994) Proc. Nat. Acad. Sci. USA 91:3809-3813; Schier et al. (1995) Gene 169:147-155; Yelton et al. (1995) J. Immunol. 155:1994-2004; Jackson et al. (1995) J. Immunol. 154(7):3310-9; and Hawkins et al. (1992) J. Mol. Biol. 226:889-896. Mutation at selective mutagenesis positions, contact or hypermutation positions, with an activity enhancing amino acid residue is described in U.S. Pat. No. 6,914,128.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Antibody: The terms “antibody” and “immunoglobulin” (abbreviated “Ig”) are used interchangeably herein and refer to a molecule comprising at least one immunoglobulin domain that specifically binds to, or is immunologically reactive with, a particular antigen. The term includes whole antibodies and any antigen binding portion or single chains thereof and combinations thereof (e.g., bispecific antibodies).

A typical antibody comprises at least two heavy chains (“HC”) and two light chains (“L”) interconnected by disulfide bonds.

Each “heavy chain” is comprised of a “heavy chain variable region” (abbreviated herein as “VH”) and a “heavy chain constant region” (abbreviated herein as “CH”). The heavy chain constant region in an unmodified antibody is comprised of three constants domains, CH1, CH2, and CH3.

Each “light chain” is comprised of a “light chain variable region” (abbreviated herein as “VL”) and a “light chain constant region”. The light chain constant region in an unmodified antibody is comprised of one constant domain, “CL”. The VH and VL regions can be further subdivided into regions of hypervariability, termed Complementarity Determining Regions (“CDR”), interspersed with regions that are more conserved, termed “framework regions” (“FW”).

Each VH and VL is composed of three CDRs and four FWs, arranged from amino-terminus to carboxy-terminus in the following order: FW1, CDR1, FW2, CDR2, FW3, CDR3, FW4. The present disclosure presents VH and VL sequences as well as the subsequences corresponding to CDR1, CDR2, and CDR3. Accordingly, a person skilled in the art would understand that the sequences of FW1, FW2, FW3 and FW4 are equally disclosed. For a particular VH, FW1 is the subsequence between the N-terminus of the VH and the N-terminus of VH-CDR1, FW2 is the subsequence between the C-terminus of VH-CDR1 and the N-terminus of VH-CDR2, FW3 is the subsequence between the C-terminus of VH-CDR2 and the N-terminus of VH-CDR3, and FW4 is the subsequence between the C-terminus of VH-CDR3 and the C-terminus of the VH. Similarly, for a particular VL, FW1 is the subsequence between the N-terminus of the VL and the N-terminus of VL-CDR1, FW2 is the subsequence between the C-terminus of VL-CDR1 and the N-terminus of VL-CDR2, FW3 is the subsequence between the C-terminus of VL-CDR2 and the N-terminus of VL-CDR3, and FW4 is the subsequence between the C-terminus of VL-CDR3 and the C-terminus of the VL.

The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. Exemplary antibodies of the present disclosure include typical antibodies, scFvs, and combinations thereof where, for example, an scFv is covalently linked (for example, via peptidic bonds or via a chemical linker) to the N-terminus of either the heavy chain and/or the light chain of a typical antibody, or intercalated in the heavy chain and/or the light chain of a typical antibody.

As used herein, the term “antibody” encompasses intact polyclonal antibodies, intact monoclonal antibodies, antibody fragments (such as Fab, Fab′, F(ab′)2, and Fv fragments), single chain variable fragment (scFv), disulfide stabilized scFvs, multispecific antibodies such as bispecific antibodies generated from at least two intact antibodies and/or antigen binding portions thereof, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity.

An antibody can be of any the five major classes (isotypes) of immunoglobulins: IgA, IgD, IgE, IgG, and IgM, or subclasses thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as therapeutic agents or diagnostic agents to form immunoconjugates.

There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al. (1997) J. Molec. Biol. 273:927-948)). In addition, combinations of these two approaches are sometimes used in the art to determine CDRs. The Kabat numbering system is generally used when referring to a residue in the variable domain (approximately residues 1-107 of the light chain and residues 1-113 of the heavy chain) (e.g., Kabat et al., Sequences of Immunological Interest, 5th Ed. Public Health Service, National Institutes of Health, Bethesda, Md. (1991)).

The phrases “amino acid position numbering as in Kabat,” “Kabat position,” and grammatical variants thereof refer to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence can contain fewer or additional amino acids corresponding to a shortening of, or insertion into, a FW or CDR of the variable domain. For example, a heavy chain variable domain can include a single amino acid insert (residue 52a according to Kabat) after residue 52 of H2 and inserted residues (e.g., residues 82a, 82b, and 82c, etc. according to Kabat) after heavy chain FW residue 82. See TABLE 1.

TABLE 1

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |
| | | (Kabat Numbering) | |
| H1 | H31-H35 | H26-H35 | H26-H32 |
| | | (Chothia Numbering) | |
| H2 | H50-H65 | H50-H58 | H52-H56 |
| H3 | H95-H102 | H95-H102 | H95-H102 |

The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

IMGT (ImMunoGeneTics) also provides a numbering system for the immunoglobulin variable regions, including the CDRs. See e.g., Lefranc, M. P. et al., Dev. Comp. Immunol. 27: 55-77(2003), which is herein incorporated by reference. The IMGT numbering system was based on an alignment of more than 5,000 sequences, structural data, and characterization of hypervariable loops and allows for easy comparison of the variable and CDR regions for all species. According to the IMGT numbering schema VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97.

For all heavy chain constant region amino acid positions discussed in the present invention, numbering is according to the EU index first described in Edelman et al., 1969, Proc. Natl. Acad. Sci. USA 63(1):78-85, describing the amino acid sequence of myeloma protein EU, which is the first human lgG1 sequenced. The EU index of Edelman et al. is also set forth in Kabat et al., 1991, Sequences of Proteins of Immunological Interest, 5th Ed., United States Public Health Service, National Institutes of Health, Bethesda. Thus, the phrases “EU index as set forth in Kabat” or “EU index of Kabat” and “position . . . according to the EU index as set forth in Kabat,” and grammatical variants thereof refer to the residue numbering system based on the human igG1 EU antibody of Edelman et al. as set forth in Kabat 1991.

The numbering system used for the variable domains (both heavy chain and light chain) and light chain constant region amino acid sequence is that set forth in Kabat 1991.

As used herein the Fc region includes the polypeptides comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM Fc can include the J chain. For IgG, Fc comprises immunoglobulin domains Cgamma2 and Cgamma3 (Cγ2 and Cγ3) and the hinge between Cgamma1 (Cγ1) and Cgamma2 (Cγ2).

Although the boundaries of the Fc region can vary, the human IgG heavy chain Fc region is usually defined to comprise residues C226 or P230 to its carboxyl-terminus, wherein the numbering is according to the EU index as set forth in Kabat. Fc can refer to this region in isolation, or this region in the context of an antibody, antibody fragment, or Fc fusion protein.

Polymorphisms have been observed at a number of different positions within antibody constant regions (e.g., Fc positions, including but not limited to positions 270, 272, 312, 315, 356, and 358 as numbered by the EU index as set forth in Kabat), and thus slight differences between the presented sequence and sequences in the prior art can exist. Polymorphic forms of human immunoglobulins have been well-characterized. At present, 18 Gm allotypes are known: G1m (1, 2, 3, 17) or G1m (a, x, f, z), G2m (23) or G2m (n), G3m (5, 6, 10, 11, 13, 14, 15, 16, 21, 24, 26, 27, 28) or G3m (b1, c3, b3, b0, b3, b4, s, t, g1, c5, u, v, g5). See Lefranc, et al., The human IgG subclasses: molecular analysis of structure, function and regulation. Pergamon, Oxford, pp. 43-78 (1990); Lefranc et al. (1979) Hum. Genet.: 50, 199-211. It is specifically contemplated that the antibodies of the present invention may be incorporate any allotype, isoallotype, or haplotype of any immunoglobulin gene, and are not limited to the allotype, isoallotype or haplotype of the sequences provided herein.

Antibody binding site: The term “antibody binding site” refers to a region in the antigen (e.g., FIXa or FXz) comprising a continuous or discontinuous site (i.e., an epitope) to which a complementary antibody specifically binds. Thus, the antibody binding site can contain additional areas in the antigen which are beyond the epitope and which can determine properties such as binding affinity and/or stability, or affect properties such as antigen enzymatic activity or dimerization. Accordingly, even if two antibodies bind to the same epitope within an antigen, if the antibody molecules establish distinct intermolecular contacts with amino acids outside of the epitope, such antibodies are considered to bind to distinct antibody binding sites.

Antigen binding portion: As used herein the term “antigen binding portion” can be interchangeably used with “antigen binding fragment” and refers to a portion of an intact antibody capable of specific binding to the same epitope as the intact antibody. In particular, it refers to a portion or portions of an intact antibody comprising one or more CDRs of an intact antibody. It is known in the art that the antigen binding function of an antibody can be performed by fragments of a full-length antibody. Examples of antibody fragments include, but are not limited to Fab, Fab′, F(ab′)2, and Fv fragments, linear antibodies, single chain antibodies, and multispecific antibodies formed from antibody fragments.

Antigen binding molecule: The terms “antigen binding molecule” and “binding molecule” are used interchangeable in the present disclosure and encompass antibodies as defined herein, as well as other molecular entities comprising at least one of the CDRs of the antibodies disclosed herein which are capable of binding to the same epitopes. For example, the term includes antibody mimics based on the scaffold of the fibronectin type III domain (monobodies), other scaffolding systems (e.g., tenascin) in which one or more CDRs are grafted, aptamers, etc. In some aspects, the antigen binding molecule can be bispecific, i.e., a “bispecific binding molecule” or a “bispecific molecule”.

Approximately: As used herein, the term “approximately,” as applied to one or more values of interest, refers to a value that is similar to a stated reference value. In certain embodiments, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein with respect to a disease, the term “associated with” means that the symptom, measurement, characteristic, or status in question is linked to the diagnosis, development, presence, or progression of that disease. As association may, but need not, be causatively linked to the disease.

When used with respect to two or more moieties, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Binding affinity: “Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure.

The terms “higher binding affinity” or “greater affinity” as applied to any of the antibodies of the invention refer to increased binding affinity (as measured, for example, by the K_{D}) with respect to a reference antibody. In some embodiments, the reference antibody is an corresponding antibody that has not been affinity matured. In some embodiments, the reference antibody is another antibody with the same specificity (e.g., for an anti-FIXa disclosed herein, a reference antibody could be another anti-FIX or anti-FIXa antibody known in the art). In some embodiments, increased binding affinity can be for example, at least about 10%, at least about 20%, at least about 30%, at least about 40%, at least about 50%, at least about 60%, at least about 70%, at least about 80%, at least about 90%, or at least about 100% higher than the binding affinity of the reference antibody for the same coagulation factor (e.g., FIX or FX), form of the factor (e.g., FIXa or FXz), or antigen binding site (e.g., epitope). In some embodiments, increased binding affinity can be for example, at least about 2-fold, at least about 3-fold, at least about 4-fold, at least about 5-fold, at least about 6-fold, at least about 7-fold, at least about 8-fold, at least about 9-fold, or at least about 10-fold higher than the binding affinity of the reference antibody for the same coagulation factor (e.g., FIX or FX), form of the factor (e.g., FIXa or FXz), or antigen binding site (e.g., epitope).

Binding: The term “binding” refers to a physical interaction between two molecules, for example, an antibody and an antigen.

(i) Binding specificity: The term “specificity” refers to the ability of an binding molecule, e.g., an antibody, to bind preferentially to one antigenic site (e.g., an epitope) versus a different antigenic site and does not necessarily imply high affinity. The terms “binding specificity” and “specificity” are used interchangeably and can refer both to (i) a specific portion of a binding molecule and (ii) the ability of the binding molecule to specifically bind (see definition of “specific binding” below) to a particular epitope. For example, in some embodiments, a bispecific antibody disclosed herein comprises two binding specificities, a first binding specificity for example to FIXa and a second binding specificity for example to FXz (in this context, a “binding specificity” such as a specific region of a bispecific antibody binding to the a particular antigenic determinant would be equivalent to a “binding domain”).
(ii) Specific binding: An binding molecule, e.g., an antibody, “specifically binds” when there is an immunological reaction of specific interaction between an antigen and the binding molecule. The term “specifically binds” means that the antibody has been generated to bind to the antigen through its variable region. The term “non-specific binding” means that the antibody has not been generated to specifically bind to the antigen but does somehow bind the antigen through non-specific means. As one example, an antibody will non-specifically bind to an Fc receptor through the Fc portion of the antibody molecule. As another example, certain antibodies may inadvertently cross-react with antigens to which they were not generated.
(iii) Preferential binding: A binding molecule, e.g., an antibody, “preferentially binds” to an antigen if it binds with greater affinity, avidity, more readily, and/or with greater duration than it binds to other substances. For example, an antibody that preferentially binds to an FIXa epitope is an antibody that binds this epitope with greater affinity, avidity, more readily, and/or with greater duration than it binds to other FIXa epitopes or non-FIXa epitopes. For example, an anti-FIX antibody preferentially binds to activated FIX over FIX zymogen if more than 50%, 60%, 70%, 80%, 90%, or 95% of the anti-FIX antibody binds to FIXa in the presence of both FIXa and FIXz. It is also understood by reading this definition that, for example, an antibody (or moiety or epitope) that preferentially binds to a first target may or may not preferentially bind to a second target. As such, “preferential binding” does not necessarily require (although it can include) exclusive binding. Thus, in some aspects, “preferential binding” can be “exclusive binding.” To exemplify these concepts, if 50% of an anti-FIX specifically binds to FIX zymogen and 50% specifically binds to FIXa, such binding would be “non-selective” or “non-preferential.” If less than 50% of the anti-FIX binds to FIX zymogen and more than 50% binds to FXa, the anti-FIX would “preferentially bind” to FIXa. If the anti-FIX does not bind to FIX zymogen and only binds to FIXa, the anti-FIX would “exclusively bind” to FIXa.

Biological sample: The term “biological sample” as used herein refers to any sample obtained from an subject, cell line, tissue culture, or other source potentially comprising a molecule comprising an antigen specifically recognized by the binding molecules disclosed herein. In some aspects, the biological sample is a blood sample, or a sample derived from a blood sample (e.g., plasma). Methods for obtaining tissue biopsies and body fluids from mammals are well known in the art.

Bispecific antibody: A “bispecific antibody” is a particular type of “bispecific molecule” or “bispecific binding molecule.” The term “bispecific antibody” means an antibody that is able to bind to at least two antigenic determinants (e.g., epitopes) through two different antigen binding sites. In certain embodiments, the bispecific antibody is capable of concurrently binding two antigenic determinants (e.g., epitopes). In some embodiments, a bispecific antibody binds one antigen (or epitope) on one of its binding arms (one pair of heavy chain/light chain), and binds a different antigen (or epitope) on its second binding arm (a different pair of heavy chain/light chain). In some embodiments, a bispecific antibody can have two distinct antigen binding arms (in both specificity and CDR sequences), and is monovalent for each antigen to which it binds. Bispecific antibodies include, e.g., those generated by quadroma technology (Milstein & Cuello (1983) Nature 305(5934):537-40), by chemical conjugation of two different monoclonal antibodies (Staerz et al. (1985) Nature 314(6012):628-31), or by knob-into-hole or similar approaches which introduces mutations in the Fc region (Holliger et al. (1993) Proc. Natl. Acad. Sci. U.S.A. 90(14): 6444-6448).

A wide variety of recombinant bispecific antibody formats have been developed in the recent past, e.g. by fusion of, e.g. an IgG antibody format and single chain domains (see Kontermann R E, mAbs 4:2, (2012) 1-16). Bispecific antibodies wherein the variable domains VL and VH or the constant domains CL and CH1 are replaced by each other are described in WO2009080251 and WO2009080252.

An approach to circumvent the problem of mispaired byproducts, which is known as ‘knobs-into-holes’, aims at forcing the pairing of two different antibody heavy chains by introducing mutations into the CH3 domains to modify the contact interface. On one chain bulky amino acids were replaced by amino acids with short side chains to create a ‘hole’. Conversely, amino acids with large side chains were introduced into the other CH3 domain, to create a ‘knob’. By coexpressing these two heavy chains (and two identical light chains, which have to be appropriate for both heavy chains), high yields of heterodimer formation (‘knob-hole’) versus homodimer formation (‘hole-hole’ or ‘knob-knob’) was observed (Ridgway J B, Presta L G, Carter P; and WO1996027011). The percentage of heterodimer could be further increased by remodeling the interaction surfaces of the two CH3 domains using a phage display approach and the introduction of a disulfide bridge to stabilize the heterodimers (Merchant A. M, et al, Nature Biotech 16 (1998) 677-681; Anwell S, Ridgway J B, Wells J A, Carter P., J Mol Biol 270 (1997) 26-35). New approaches for the knobs-into-holes technology are described in e.g. in EP 1870459A1. Xie, Z., et al, J Immunol Methods 286 (2005) 95-101 refers to a format of bispecific antibody using scFvs in combination with knobs-into-holes technology for the FC part.

The modular architecture of antibodies has been exploited to create more than 60 different bispecific antibody formats. See Spiess et al. (2015) Molecular Immunology 67:95-106, which is herein incorporated by reference in its entirety. Accordingly, in some aspects, the bispecific antibody format is selected from crossMab, DAF (Dual Action Fab) (two-in-one), DAF (four-in-one), DutaMab, DT-IgG, Knobs-in-holes common LC, Knobs-in-holes assembly, Charge pair, Fab-arm exchange, SEEDbody, Triomab, LUZ-Y (bispecific antibody with a leucize zipper inducing heterodimerization of two HCs), Fcab, Kλ-body, Orthogonal Fab, DVD-IgG (dual variable domain IgG), IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, DVI-IgG (four-in-one), Nanobody, Nanobody-HSA, BiTE (bispecific T cell engager), Diabody, DART (dual-affinity-retargeting), TandAb (tandem antibody), scDiabody, scDiabody-CH3, Triple Body, Miniantibody, Minibody, TriBi minibody, scFv-CH3 KIH, Fab-scFv, scFv-CH-CL-scFv, F(ab′)2, F(ab′)2-ScFv2, scFv-KIH, Fab-scFv-Fc, Tetravalent HC Ab, scDiabody-Fc, Diabody-Fc, Tandem scFv-Fc, Intrabody, Dock and Locck, ImmTAC, HSAbody, scDiabody-HSA, Tandem scFv-Toxin, IgG-IgG, Cov-X-Body, and scFv1-PEG-scFV2.

In some aspects, the bispecific antibody is an asymmetric (e.g., heterdimeric) antibody, comprising a chain A and a chain B, wherein
(i) Chain A comprises a T336W mutation, and chain B comprises T366W, L368A, and Y407V mutations (Knows-in holes format);
(ii) Chain A comprises a F405L mutation, and chain B comprises a K409R mutation (duobody format);
(iii) Chain A comprises T350V, L351Y, F405A, and Y407V mutations, and chain B comprises T350V, T366L, K392L and T394W mutations (azymetric format);
(iv) Chain A comprises K409D and K392D mutations, and chain B comprises D399K and E356K mutations (Charge pair format);
(v) Chain A comprises D221E, P228E, and L368E mutations, and chain B comprises D221R, P228R and K409R mutations (Charge pair format);
(vi) Chain A comprises S364H and F405A mutations, and chain B comprises Y349T and T394F mutations (HA-TF format); or,
(vii) Chain A comprises an IgG/A chimera, and chain B also comprises an IgG/A chimera (SEEDbody format).

In some aspects, the bispecific antibody is a monospecific antibody engineered for bispecificity by appending either the amino or carboxy termini of either the light or heavy chains with additional antigen-binding units. Alternatives for these additional antigen-binding units include single domain antibodies (unpaired VL or VH), paired antibody variable domains (e.g., Fv or scFv) or engineered protein scaffolds. In some aspects, a bispecific molecule of the present invention comprises a bispecific antibody fragment. Numerous bispecific fragment forms lacking some or all of the bispecific antibody constant domains are known in the art. In some aspects, the bispecific molecule of the invention is a bispecific fusion protein, e.g., an ImmTAC (scFv linked to an affinity matured receptor). In other aspects, the bispecific molecule is a bispecific antibody conjugate.

Bispecific molecule: See the definition of “Antigen binding molecule”/“Binding molecule” above.

Chimeric antibody: The term “chimeric antibody” and grammatical variants thereof refers to antibodies wherein the amino acid sequence of the immunoglobulin molecule is derived from two or more animal species. Typically, the variable region of both light and heavy chains corresponds to the variable region of antibodies derived from one species of mammals (e.g., mouse, rat, rabbit, etc.) with the desired specificity, and/or affinity, and/or capability while the constant regions are homologous to the sequences in antibodies derived from another species (usually human) to avoid eliciting an immune response in that species.

Complementarity determining region: The term “complementarity determining region” or “CDR” refers to variable regions of either H (heavy) or L (light) chains contains the amino acid sequences capable of specifically binding to antigenic targets. These CDR regions account for the basic specificity of the antibody for a particular antigenic determinant structure. Such regions are also referred to as “hypervariable regions.” See definition of “Antibody” above.

Conservative amino acid substitution: A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. In another aspect, a string of amino acids can be conservatively replaced with a structurally similar string that differs in order and/or composition of side chain family members.

Non-conservative amino acid substitutions include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

Other amino acid substitutions can be readily identified by workers of ordinary skill. For example, for the amino acid alanine, a substitution can be taken from any one of D-alanine, glycine, beta-alanine, L-cysteine and D-cysteine. For lysine, a replacement can be any one of D-lysine, arginine, D-arginine, homo-arginine, methionine, D-methionine, ornithine, or D-ornithine. Generally, substitutions in functionally important regions that can be expected to induce changes in the properties of isolated polypeptides are those in which (i) a polar residue, e.g., serine or threonine, is substituted for (or by) a hydrophobic residue, e.g., leucine, isoleucine, phenylalanine, or alanine; (ii) a cysteine residue is substituted for (or by) any other residue; (iii) a residue having an electropositive side chain, e.g., lysine, arginine or histidine, is substituted for (or by) a residue having an electronegative side chain, e.g., glutamic acid or aspartic acid; or (iv) a residue having a bulky side chain, e.g., phenylalanine, is substituted for (or by) one not having such a side chain, e.g., glycine. The likelihood that one of the foregoing non-conservative substitutions can alter functional properties of the protein is also correlated to the position of the substitution with respect to functionally important regions of the protein: some non-conservative substitutions can accordingly have little or no effect on biological properties.

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some embodiments, two or more sequences are said to be “completely conserved” or “identical” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an polynucleotide or polypeptide or may apply to a portion, region or feature thereof.

Cross-compete: The terms “compete” or “cross-compete”, as used herein with regard to a binding molecule, e.g., an antibody, means that a first binding molecule, e.g., a first antibody or an antigen-binding portion thereof, binds to an epitope in a manner sufficiently similar to the binding of a second binding molecule, e.g., a second antibody or an antigen-binding portion thereof, such that the result of binding of the first binding molecule with its cognate epitope is detectably decreased in the presence of the second binding molecule compared to the binding of the first binding molecule in the absence of the second binding molecule. The alternative, where the binding of the second binding molecule to its epitope is also detectably decreased in the presence of the first binding molecule, can, but need not be the case. That is, a first binding molecule can inhibit the binding of a second binding molecule to its epitope without that second molecule inhibiting the binding of the first binding molecule to its respective epitope. However, where each binding molecule detectably inhibits the binding of the other binding molecule with its cognate epitope, whether to the same, greater, or lesser extent, the binding molecules are said to “cross-compete” with each other for binding of their respective epitope(s). Both competing and cross-competing binding molecules are encompassed by the present invention.

Binding molecules, e.g., antibodies, are said to “bind to the same epitope” or “comprising the same binding site” or have “essentially the same binding” characteristics, if the binding molecules cross-compete so that only one antibody can bind to the epitope at a given point of time, i.e., one binding molecule prevents the binding or modulating effect of the other.

Competition herein means a greater relative inhibition than at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% as determined by competition ELISA analysis or by ForteBio analysis, e.g., as described in the Examples section. It may be desirable to set a higher threshold of relative inhibition as criteria of what is a suitable level of competition in a particular context. Thus, for example, it is possible to set criteria for the competitive binding, wherein at least about 40% relative inhibition is detected, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or even about 100%, before an antibody is considered sufficiently competitive.

Effective Amount: As used herein, the term “effective amount” of an agent, e.g., a therapeutic agent such as an antibody, is that amount sufficient to effect beneficial or desired results, for example, clinical results, and, as such, an “effective amount” depends upon the context in which it is being applied. For example, in the context of administering a therapeutic agent that treats bleeding, an effective amount of an agent is, for example, an amount sufficient to reduce or decrease in bleeding occurrences, as compared to the response obtained without administration of the agent. The term “effective amount” can be used interchangeably with “effective dose,” “therapeutically effective amount,” or “therapeutically effective dose.”

Effector function: The “effector function” of an antibody is the ability to bind complement proteins which can assist in lysing the target antigen, for example, a cellular pathogen, in a process termed complement-dependent cytotoxicity (CDC). Another effector activity of the Fc region is to bind to Fc receptors (e.g., FcγRs) on the surface of immune cells, or so-called effector cells, which have the ability to trigger other immune effects. The effector function of an antibody can be avoided, e.g., by using antibody fragments lacking the Fc region (e.g., such as a Fab, F(ab′)2, or single chain Fv (scFv)), by removing sugars that are linked to particular residues in the Fc region (aglycosylated antibodies), or by employing Fc regions from an IgG4 antibody (an “effectorless IgG4 Fc”), instead of IgG1. It is well known that IgG4 antibodies are characterized by having lower levels of complement activation and antibody-dependent cellular cytotoxicity than IgG1.

Engineered antibody: As used herein, embodiments of the invention are “engineered” when they are designed to have a feature or property, whether structural or chemical, that varies from a starting point, wild type, or native molecule. In this respect, an “engineered antibody” is, for example, an antibody in which substitutions/mutations have been made to improve affinity, plasma half-life, etc., the format of the antibody has been modified (e.g., by generating an scFv or a bispecific antibody), or the antibody has been subjected to affinity maturation.

Epitope: The term “epitope” as used herein refers to an antigenic protein determinant (e.g., an amino acid subsequence of FIXa or FXz) capable of binding to a binding molecule, e.g., an antibody. Epitopes usually consist of chemically active surface groupings of molecules such as amino acids or sugar side chains and usually have specific three dimensional structural characteristics, as well as specific charge characteristics. The part of an antibody or binding molecule that recognizes the epitope is called a paratope. The epitopes of protein antigens are divided into two categories, conformational epitopes and linear epitopes, based on their structure and interaction with the paratope. A conformational epitope is composed of discontinuous sections of the antigen's amino acid sequence. These epitopes interact with the paratope based on the 3-D surface features and shape or tertiary structure of the antigen. By contrast, linear epitopes interact with the paratope based on their primary structure. A linear epitope is formed by a continuous sequence of amino acids from the antigen.

Expression vector: An “expression vector” is a polynucleotide which, when introduced into an appropriate host cell, can be transcribed and translated into a polypeptide. An “expression system” usually refers to a suitable host cell comprised of an expression vector that can function to yield a desired expression product. The antibodies (e.g., bispecific antibodies) according to the invention are preferably produced by recombinant means. Such methods are widely known in the state of the art and comprise protein expression in prokaryotic and eukaryotic cells with subsequent isolation of the antibody polypeptide and usually purification to a pharmaceutically acceptable purity.

Germline sequence: As used herein, the term “germline sequence” refers to a sequence of unrearranged immunoglobulin DNA sequences. Any suitable source of unrearranged immunoglobulin may be used. The term “germline” refers to the sequences of the V, D, and J minigenes, prior to the exposure of an antibody to an antigen. Rearranged “V-regions” describe the genetic element which results from the rearrangement event between V, D, and J (for heavy chains) or V and J minigenes (for light chains). An “antibody V-region” refers to the polypeptide region encoded by the V, D, and J element. An antibody V-region is encoded by rearranged V, D, and J minigenes. The term “V(D)J Recombination” refers to any process wherein a V, D, or J minigene is recombined to another V, D, or J minigene. A V-region may be part of a full length antibody, an Fab, a scFv, or any other derivative of an antibody (see definition of antibody below). A “germline V-region” refers to the sequence of rearranged V, D, and J minigenes prior to significant mutagenic events. A germline V-region may have random insertions or deletions at the junctions of the V-D, D-J, or V-J minigenes. A non-germline V-region (or a “mature” V-region) will differ from the germline sequences of the minigenes by usually more than 5 residues (not including the junctional deletions or insertions).

Homology: As used herein, the term “homology” refers to the overall relatedness between polymeric molecules, e.g. between nucleic acid molecules (e.g. DNA molecules and/or RNA molecules) and/or between polypeptide molecules. Generally, the term “homology” implies an evolutionary relationship between two molecules. Thus, two molecules that are homologous will have a common evolutionary ancestor. In the context of the present invention, the term homology encompasses both to identity and similarity.

In some embodiments, polymeric molecules are considered to be “homologous” to one another if at least 25%, 30%, 35%, 40%, 45%, 50%, 55%, 60%, 65%, 70%, 75%, 80%, 85%, 90%, 95%, or 99% of the monomers in the molecule are identical (exactly the same monomer) or are similar (conservative substitutions). The term “homologous” necessarily refers to a comparison between at least two sequences (polynucleotide or polypeptide sequences).

Human antibody: The term “human antibody” means an antibody produced by a human or an antibody having an amino acid sequence corresponding to an antibody produced by a human made using any technique known in the art (e.g., recombinant expression in cultures cells, or expression in transgenic animals). Thus, the term human antibody also encompasses an antibody having an amino acid sequence corresponding to an antibody originally produced by a human (or an engineered variant or derivative thereof) but expressed in a non-human system (e.g., produced by chemical synthesis; recombinantly expressed in microbial, mammal, or insect cells; or expressed in an animal subject). Accordingly, an antibody obtained from a human subject or from human cells (e.g., hybridoma or cell line expressing a recombinant antibody or fragment thereof) and subsequently expressed in an animal, e.g., mice, is considered a human antibody. This definition of a human antibody includes intact or full-length antibodies, fragments thereof, and/or antibodies comprising at least one human heavy and/or light chain polypeptide such as, for example, an antibody comprising murine light chain and human heavy chain polypeptides.

Humanized antibody: The term “humanized antibody” refers to an antibody derived from a non-human (e.g., murine) immunoglobulin, which has been engineered to contain minimal non-human (e.g., murine) sequences. Typically, humanized antibodies are human immunoglobulins in which residues from the CDRs are replaced by residues from the CDRs of a non-human species (e.g., mouse, rat, rabbit, or hamster) that have the desired specificity, affinity, and capability (Jones et al., 1986, Nature, 321:522-525; Riechmann et al., 1988, Nature, 332:323-327; Verhoeyen et al., 1988, Science, 239:1534-1536). In some instances, the FW residues of a human immunoglobulin are replaced with the corresponding residues in an antibody from a non-human species that has the desired specificity, and/or affinity, and/or capability.

The humanized antibody can be further modified by the substitution of additional residues either in the FW regions and/or within the replaced non-human residues to refine and optimize antibody specificity, and/or affinity, and/or capability. In general, the humanized antibody will comprise substantially all of at least one, and typically two or three, variable domains containing all or substantially all of the CDR regions that correspond to the non-human immunoglobulin whereas all or substantially all of the FW regions are those of a human immunoglobulin consensus sequence. The humanized antibody can also comprise at least a portion of an immunoglobulin constant region or domain (Fc), typically that of a human immunoglobulin. Examples of methods used to generate humanized antibodies are described in U.S. Pat. Nos. 5,225,539 or 5,639,641.

Identity: As used herein, the term “identity” refers to the overall monomer conservation between polymeric molecules, e.g., between polypeptide molecules or polynucleotide molecules (e.g. DNA molecules and/or RNA molecules). The term “identical” without any additional qualifiers, e.g., protein A is identical to protein B, implies the sequences are 100% identical (100% sequence identity). Describing two sequences as, e.g., “70% identical,” is equivalent to describing them as having, e.g., “70% sequence identity.”

Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. When comparing DNA and RNA, thymine (T) and uracil (U) can be considered equivalent.

Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity (% ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

Immunoconjugate: The term “immunoconjugate” as used herein refers to a compound comprising a binding molecule (e.g., an anti-FIXa, anti-FXz, or biospecific anti-FIXa/anti-FXz) and one or more moieties, e.g., therapeutic or diagnostic moieties, chemically conjugated to the binding molecule. In general an immunoconjugate is defined by a generic formula: A-(L-M)n wherein A is a binding molecule (e.g., an antibody), L is an optional linker, and M is a heterologous moiety which can be for example a therapeutic agent, a detectable label, etc., and n is an integer. Immunoconjugates can also be defined by the generic formula in reverse order. In some aspects, the immunoconjugate is an “antibody-Drug Conjugate” (“ADC”). In the context of the present disclosure the term “immunoconjugate” is not limited to chemically or enzymatically conjugates molecules. The term “immunoconjugate” as used in the present disclosure also includes genetic fusions.

Isolated: As used herein, the term “isolated” refers to a substance or entity (e.g., polypeptide, antibody, polynucleotide, vector, cell, or composition which is in a form not found in nature) that has been separated from at least some of the components with which it was associated (whether in nature or in an experimental setting). Isolated substances (e.g., nucleotide sequence or protein sequence) can have varying levels of purity in reference to the substances from which they have been associated.

Isolated substances and/or entities can be separated from at least about 10%, at least about 15%, at least about 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least 95%, or more of the other components with which they were initially associated.

In some embodiments, isolated agents are more than about 80%, about 85%, about 90%, about 91%, about 92%, about 93%, about 94%, about 95%, about 96%, about 97%, about 98%, about 99%, or more than about 99% pure.

As used herein, a substance is “pure” if it is substantially free of other components. The term “substantially isolated” means that the compound is substantially separated from the environment in which it was formed or detected. Partial separation can include, for example, a composition enriched in the compound of the present disclosure. Substantial separation can include compositions containing at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 97%, or at least about 99% by weight of the compound of the present disclosure, or salt thereof.

A polynucleotide (e.g., an antibody), vector, polypeptide, cell, or any composition disclosed herein which is “isolated” is a polynucleotide (e.g., an antibody), vector, polypeptide, cell, or composition which is in a form not found in nature. Isolated polynucleotides, vectors, polypeptides, or compositions include those which have been purified to a degree that they are no longer in a form in which they are found in nature. In some aspects, a polynucleotide, vector, polypeptide, or composition which is isolated is substantially pure.

Mimic FVIIIa activity: The ability of a binding molecule disclosed herein to “mimic FVIIIa activity,” i.e., the ability to mimic the activity of activated factor VIII, can be measured according to different methods known in the art. One such method is a chromogenic assay as described in the Examples section of this specification. In one aspect, a binding molecule disclosed herein (e.g., a bispecific antibody) is said to “mimic FVIIIa activity” is there is an observed rate of FXa substrate cleavage at least three standard deviations above the mean basal rate in the absence of the added binding molecule (e.g., a bispecific antibody). Another exemplary method is an activated Partial Thromboplastin Time (aPTT) assay. The term ‘Activated Partial Thromboplastin Time (APTT)’ derives from the original form of the test (devised in 1953) in which only the phospholipid concentration of the test was controlled (as opposed to the phospholipid and the surface activator concentrations) and the name ‘partial thromboplastin’ was applied at the time to phospholipid preparations which accelerated clotting but did not correct the prolonged clotting times of haemophilic plasma. Essentially the term ‘partial’ means phospholipid is present but no Tissue Factor. The aPIT is also known as: Kaolin Cephalin Clotting Time (KCCT) or Partial Thromboplastin Time with Kaolin (PTTK). Other useful methods include a one stage (OS) clotting assay which is modified from the traditional aPIT assay described above. The one stage clotting assay uses FVIII-deficient plasma and a diluted test sample, and can be quantitative for FVIII activity. See Example 4. In contrast, the aPTT assay uses sample plasma with the aPIT reagent and calcium and reports the clotting time.

Monoclonal Antibody: A “monoclonal antibody” refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, Fv), single chain variable fragments (scFv), fusion proteins comprising an antibody portion, and any other modified immunoglobulin molecule comprising an antigen recognition site. Furthermore, “monoclonal antibody” refers to such antibodies made in any number of ways including, but not limited to, by hybridoma, phage selection, recombinant expression, and transgenic animals (e.g., expression of a human antibody in a transgenic mouse).

Mutation: In the content of the present disclosure, the terms “mutation” and “amino acid substitution” as defined above (sometimes referred simply as a “substitution”) are considered interchangeable. In some aspects, the term mutation refers to the deletion, insertion, or substitution of any nucleotide, by chemical, enzymatic, or any other means, in a nucleic acid encoding an antibody germline gene such that the amino acid sequence of the resulting polypeptide is altered at one or more amino acid residues. In some aspects, a mutation in a nucleic acid sequence disclosed herein results in an amino acid substitution. In other aspects, the mutation of a codon in a nucleic acid sequence disclosed herein wherein the resulting codon is a synonymous codon does not result in an amino acid substitution. Accordingly, in some aspects, the nucleic acid sequences disclosed herein can be codon optimized by introducing one or more synonymous codon changes. Such codon optimization can, for example, (i) improve protein yield in recombinant protein expression, or (ii) improve the stability, half life, or other desirable property of an mRNA or a DNA encoding a binding molecule disclosed herein, wherein such mRNA or DNA is administered to a subject in need thereof.

Patient: As used herein, “patient” refers to a subject who may seek or be in need of treatment, requires treatment, is receiving treatment, will receive treatment, or a subject who is under care by a trained professional for a particular disease or condition.

Pharmaceutical composition: The term “pharmaceutical composition” refers to a preparation which is in such form as to permit the biological activity of the active ingredient (e.g., a binding molecule disclosed herein, such as an antibody) to be effective, and which contains no additional components which are unacceptably toxic to a subject to which the composition would be administered. Such composition can be sterile.

Pharmaceutically acceptable: The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio. In general, approval by a regulatory agency of the Federal or state governments (or listed in the U.S. Pharmacopeia or other generally recognized pharmacopeia) for use in animals, and more particularly in humans implies that those compounds, materials, compositions, and/or dosage forms are pharmaceutically acceptable. Compounds, materials, compositions, and/or dosage forms that are generally acceptable as safe for therapeutically purposes are “therapeutically acceptable.” Compounds, materials, compositions, and/or dosage forms that are generally acceptable as safe for diagnostic purposes are “diagnostically acceptable.”

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients can include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Excipients that are generally accepted as safe for therapeutic purposes are “therapeutically acceptable excipients.” Excipients that are generally accepted as safe for diagnostic purposes are “diagnostically acceptable excipients.”

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are used. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Pharmaceutically acceptable solvate: The term “pharmaceutically acceptable solvate,” as used herein, means a compound of the invention wherein molecules of a suitable solvent are incorporated in the crystal lattice. A suitable solvent is physiologically tolerable at the dosage administered. For example, solvates can be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Pharmacokinetic: As used herein, “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Polynucleotide: The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In particular aspects, the polynucleotide comprises an mRNA. In other aspect, the mRNA is a synthetic mRNA. In some aspects, the synthetic mRNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine). In some aspects, the polynucleotide (e.g., a synthetic RNA or a synthetic DNA) comprises only natural nucleobases, i.e., A, C, T and U in the case of a synthetic DNA, or A, C, T, and U in the case of a synthetic RNA.

The skilled artisan will appreciate that the T bases in the codon maps disclosed herein are present in DNA, whereas the T bases would be replaced by U bases in corresponding RNAs. For example, a codon-nucleotide sequence disclosed herein in DNA form, e.g., a vector or an in-vitro translation (IVT) template, would have its T bases transcribed as U based in its corresponding transcribed mRNA. In this respect, both codon-optimized DNA sequences (comprising T) and their corresponding RNA sequences (comprising U) are considered codon-optimized nucleotide sequence of the present invention. A skilled artisan would also understand that equivalent codon-maps can be generated by replaced one or more bases with non-natural bases. Thus, e.g., a TTC codon (DNA map) would correspond to a UUC codon (RNA map), which in turn would correspond to a ΨΨC codon (RNA map in which U has been replaced with pseudouridine).

Standard A-T and G-C base pairs form under conditions which allow the formation of hydrogen bonds between the N3-H and C4-oxy of thymidine and the Ni and C6-NH₂, respectively, of adenosine and between the C2-oxy, N3 and C4-NH₂, of cytidine and the C2-NH₂, N′-H and C6-oxy, respectively, of guanosine. Thus, for example, guanosine (2-amino-6-oxy-9-β-D-ribofuranosyl-purine) can be modified to form isoguanosine (2-oxy-6-amino-9-β-D-ribofuranosyl-purine). Such modification results in a nucleoside base which will no longer effectively form a standard base pair with cytosine. However, modification of cytosine (1-β-D-ribofuranosyl-2-oxy-4-amino-pyrimidine) to form isocytosine (1-β-D-ribofuranosyl-2-amino-4-oxy-pyrimidine-) results in a modified nucleotide which will not effectively base pair with guanosine but will form a base pair with isoguanosine (U.S. Pat. No. 5,681,702 to Collins et al.). Isocytosine is available from Sigma Chemical Co. (St. Louis, Mo.); isocytidine can be prepared by the method described by Switzer et al. (1993) Biochemistry 32:10489-10496 and references cited therein; 2′-deoxy-5-methyl-isocytidine can be prepared by the method of Tor et al. (1993) J. Am. Chem. Soc. 115:4461-4467, and references cited therein; and isoguanine nucleotides can be prepared using the method described by Switzer et al., 1993, supra, and Mantsch et al. (1993) Biochem. 14:5593-5601, or by the method described in U.S. Pat. No. 5,780,610 to Collins et al. Other nonnatural base pairs can be synthesized by the method described in Piccirilli et al. (1990) Nature 343:33-37, for the synthesis of 2,6-diaminopyrimidine and its complement (1-methylpyrazolo-[4,3]pyrimidine-5,7-(4H,6H)-dione. Other such modified nucleotide units which form unique base pairs are known, such as those described in Leach et al. (1992) J. Am. Chem. Soc. 114:3675-3683 and Switzer et al., supra.

Polypeptide: The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, omithine, p-acetylphenylalanine, D-amino acids, and creatine), as well as other modifications known in the art.

The term, as used herein, refers to proteins, polypeptides, and peptides of any size, structure, or function. Polypeptides include gene products, naturally occurring polypeptides, synthetic polypeptides, homologs, orthologs, paralogs, fragments and other equivalents, variants, and analogs of the foregoing. A polypeptide can be a single polypeptide or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some embodiments, a “peptide” can be less than or equal to 50 amino acids long, e.g., about 5, 10, 15, 20, 25, 30, 35, 40, 45, or 50 amino acids long.

Preventing: As used herein, the term “preventing” refers to partially or completely delaying onset of an disease, disorder and/or condition; partially or completely delaying onset of one or more symptoms, features, or clinical manifestations of a particular disease, disorder, and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition.

Prophylactic: As used herein, “prophylactic” refers to a therapeutic or course of action used to prevent the onset of a disease or condition, or to prevent or delay a symptom associated with a bleeding episode, e.g., hemophilia.

Prophylaxis: As used herein, a “prophylaxis” refers to a measure taken to maintain health and prevent or delay the onset of a bleeding episode, or to prevent or delay symptoms associated with a disease or condition.

Recombinant: A “recombinant” polypeptide or protein refers to a polypeptide or protein produced via recombinant DNA technology. Recombinantly produced polypeptides and proteins expressed in engineered host cells are considered isolated for the purpose of the invention, as are native or recombinant polypeptides which have been separated, fractionated, or partially or substantially purified by any suitable technique. The polypeptides disclosed herein can be recombinantly produced using methods known in the art. Alternatively, the proteins and peptides disclosed herein can be chemically synthesized.

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Subject: By “subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; bears, food animals such as cows, pigs, and sheep; ungulates such as deer and giraffes; rodents such as mice, rats, hamsters and guinea pigs; and so on. In certain embodiments, the mammal is a human subject. In other embodiments, a subject is a human patient. In a particular embodiment, a subject is a human patient or cells thereof whether in vivo, in vitro or ex vivo, amenable to the methods described herein.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneous: As used herein and as it relates to plurality of doses, the term means within 2 seconds.

Suffering from: An individual who is “suffering from” a disease, disorder, and/or condition has been diagnosed with or displays one or more symptoms of the disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) can be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutic Agent: The terms “therapeutic agent” or “agent” refers to a molecular entity that, when administered to a subject, has a therapeutic, diagnostic, and/or prophylactic effect and/or elicits a desired biological and/or pharmacological effect. For example, in some embodiments, a bispecific antibody disclosed herein can be a therapeutic agent. In some embodiments, an agent is another molecule (e.g., a clotting factor, cofactor, etc.) which is co-administered as part of a combination therapy with at least one of the antibodies disclosed herein.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Treat. treatment, therapy: As used herein, the terms “treat” or “treatment” or “therapy” or grammatical variants thereof refer to partially or completely alleviating, ameliorating, improving, relieving, delaying onset of, inhibiting progression of, reducing severity of, and/or reducing incidence of one or more symptoms or features of a bleeding disease, disorder, or condition, e.g., hemophilia. For example, “treating” a bleeding disorder can refer to prevent bleeding, decrease the frequency and/or severity of bleeding episodes, etc. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Vector: A “vector” is a nucleic acid molecule, in particular self-replicating, which transfers an inserted nucleic acid molecule into and/or between host cells. The term includes vectors that function primarily for insertion of DNA or RNA into a cell (e.g., chromosomal integration), replication of vectors that function primarily for the replication of DNA or RNA, and expression vectors that function for transcription and/or translation of the DNA or RNA. In some aspects, the administration and/or expression of a nucleic acid (DNA or RNA, such as an mRNA) encoding a binding molecule disclosed herein can take place in vitro (e.g., during recombinant protein production), whereas in other cases it can take place in vivo (e.g., administration of an mRNA to a subject), or ex vivo (e.g., DNA or RNA introduced into an autologous or heterologous cells for administration to a subject in need thereof). Also included are vectors that provide more than one of the functions as described.

### II. Anti-FIX and Anti-FX Binding Molecules

The present disclosure provides antibodies that bind to factor IX and to factor X, as well as antigen binding portions thereof. These antibodies are capable of preferential binding to specific functional forms of these clotting factors. For example, in some embodiments, the disclosed antibodies against FIX preferentially bind to activated FIX (FIXa), e.g., free FIXa or FIXa covalently linked to a substrate mimic in the active site (FXa+EGR-CMK). In other embodiments, the disclosed antibodies preferentially bind to FIXa-SM over free FIXa or FIX zymogen. In yet other embodiments, the disclosed antibodies preferentially bind to free FIXa over FIXa-SM or FIX zymogen. In contrast, in some embodiments, the disclosed antibodies against FX preferentially bind to FX zymogen (FXz) over activated FX (FXa). This preferential binding is critical to generate bispecific molecules comprising an anti-FIXa moiety and an anti-FXz moiety that can specifically and simultaneously bind to FIXa and FXz. Factor VIII is a cofactor for FIXa which, in the presence of Ca²⁺ and phospholipids forms a complex with FX that converts FX to the activated FXa. Therefore, the formation of an antibody-mediated complex between FIXa and FXz mimics the effect of FVIIIa.

In still other embodiments, some disclosed antibodies preferentially bind to FIX zymogen over free FIXa or FIXa-SM (“anti-FIXz antibody”). Therefore, the anti-FIXz antibody can be used to generate a bispecific molecule comprising the anti-FIXz antibody and an anti-FX antibody (e.g., an anti-FXz antibody or an anti-FXa antibody).

In certain embodiments, some disclosed anti-FX antibodies preferentially bind to FXa over FXz (“anti-FXa antibody”). The anti-FXa antibody can be used to generate a bispecific molecule comprising the anti-FXa antibody and an anti-FIX antibody (e.g., an anti-FIXa antibody or an anti-FIXz antibody).

The formation of antibody-mediated complex between FIX and FX can therefore be used to bypass FVIII replacement therapy, in particular, in subjects having developed antibodies against FVIII or at risk of developing antibodies against FVIII.

The present disclosure also provides a bispecific binding molecule that binds to FX (FXz and/or FXa) and FIX (FIXz and/or FIXa). In one embodiment, the bispecific binding molecule can be a combination of any one of anti-FIXa antibody or anti-FIXz antibody and any one of anti-FXa antibody or anti-FXz antibody. In some embodiments, the bispecific binding molecule specifically binds to FXz, FIXz, and FIXa, but has no detectable binding to FXa. In certain embodiments, the bispecific binding molecule binds to FIXz, FIXa, and FXz with different binding affinity (e.g., K_{D}). In other embodiments, the bispecific binding molecule binds with a K_{D} less than 1 μM to each of FIXz, FIXa, and FXz (e.g., 8 nM, 2 nM, or 20 nM, respectively).

### (a) Anti-FIXa Binding Molecules

The present disclosure provides anti-FIX binding molecules, e.g., anti-FIX antibodies or molecules comprising antigen binding portions thereof, that preferentially bind activated FIX (FIXa) over FIX zymogen.

Factor IX (FIX) is synthesized by liver hepatocytes as a pre-prozymogen that requires extensive posttranslational modification. The pre-prozymogen contains a pre-peptide (hydrophobic signal peptide) at its amino terminal that transports the growing polypeptide into the lumen of the Endoplasmic Reticulum. Once inside the ER, this signal peptide is cleaved by a signal peptidase. A pro-peptide functions as a recognition element for a vitamin K-dependent carboxylase (γ-glutamyl carboxylase) which modifies 12 glutamic acid residues to gamma-carboxyglutamyl (Gla) residues. These residues are required for the association with the anionic phospholipid surface through Ca2+-dependent binding.

The amino acid sequence of pre-pro-FIX zymogen is provided below (the signal sequence is underlined (1-28); the propeptide sequence (29-46) is bolded):

(SEQ ID NO: 764)
MQRVNMIMAESPGLITICLLGYLLSAECTVFLDHE
NANKILNRPKRYNSGKLEEFVQGNLERECMEEKCS
FEEAREVFENTERTTEFWKQYVDGDQCESNPCLNG
GSCKDDINSYECWCPFGFEGKNCELDVTCNIKNGR
CEQFCKNSADNKVVCSCTEGYRLAENQKSCEPAVP
FPCGRVSVSQTSKLTRAETVFPDVDYVNSTEAETI
LDNITQSTQSFNDFTRVVGGEDAKPGQFPWQVVLN
GKVDAFCGGSIVNEKWIVTAAHCVETGVKITVVAG
EHNIEETEHTEQKRNVIRIIPHHNYNAAINKYNHD
IALLELDEPLVLNSYVTPICIADKEYTNIFLKFGS
GYVSGWGRVFHKGRSALVLQYLRVPLVDRATCLRS
TKFTIYNNMFCAGFHEGGRDSCQGDSGGPHVTEVE
GTSFLTGIISWGEECAMKGKYGIYTKVSRYVNWIK
EKTKLT

After the cleavage of the signal peptide and the propeptide, FIX is in a zymogen form. FIX zymogen thus circulates as a 415 amino acid, single chain polypeptide. See, Vysotchin et al., J. Biol. Chem. 268:8436 (1993). In one embodiment, FIX zymogen is amino acids 47 to 461 of SEQ ID NO: 764. In another embodiment, FIX zymogen is amino acids 47 to 461 of SEQ ID NO: 764, wherein amino acid residue 180 is alanine instead of arginine (i.e., non-activatable FIX).

The zymogen of FIX is activated by FXIa or by the tissue factor/FVIIa complex. The first cleavage is at Arg191 (Arg145 in the mature FIX sequence), generating an inactive FIX-alpha. The second cleavage at Arg226 (Arg180 in the mature FIX sequence) removes 35 amino acids of the FIX activation peptide and results in a catalytically active molecule FIXa-beta. This catalytically active FIXa not associated with FVIIIa is also called herein as free FIXa. This resulting heterodimer is held by a disulfide bridge at Cys178-Cys335. The serine protease contains a catalytic triad of His267, Asp315, and Ser411. Upon cleavage at Arg226, Val227 can form a salt bridge with Asp410, which is a characteristic of active serine proteases. In one embodiment, free FIXa consists of amino acids 47 to 191 of SEQ ID NO: 764 and amino acids 227 to 461 of SEQ ID NO: 764, wherein amino acid 178 and amino acid 335 of SEQ ID NO: 764 form a disulfide bond.

It is known, however, that activity of free FIXa is similar to the activity of FIX zymogen. The complex formation with cofactor FVIIIa represents a critical second phase of activation, after which the intrinsic Xase complex reaches an approximately 200,000-fold enhanced activity that is strictly specific toward the physiological substrate FX and restricted to the surface of activated platelets (van Dieijen et al., J Biol Chem. 1981 Apr. 10; 256(7):3433-42). This macromolecular activation is assisted by low-molecular-weight agonists, including Ca2+(Mathur et al., Biol. Chem., 272 (1997), pp. 23418-23426). Several lines of evidence indicate that Ca2+ binding is accompanied by a conformational rearrangement (Bajaj et al., Proc. Natl. Acad. Sci. USA, 89 (1992), pp. 152-156, Enfield and Thompson, Blood, 64 (1984), pp. 821-831). This superactive FIXa in a tenase complex can be mimicked by covalently attaching a substrate mimic (e.g., Glu-Gly-Arg-chloromethyl ketone (EGR-CMK)) to the active site of FIXa (FXa+EGR-CMK, also called as FIXa-SM). Therefore, FIXa-SM can be used as an important tool to distinguish antibodies or antigen binding portion thereof that preferentially bind to superactive FIXa (in tenase complex) compared to free FIXa.

Tripeptide chloromethylketones are generally accepted in the field as substrate mimics, with the tripeptide sequence representing the native substrate sequence that is cleaved by a particular enzyme and the CMK part allows this tripeptide to be irreversibly locked into the active site since it reacts with the active site serine. As a consequence, if an enzyme is bound to a substrate-mimic, this should represent the substrate bound form, i.e. the true active conformation. See Brandsteter et al. (1995) Proc. Natl. Acad. Sci. USA 92(21):9796-80, and Hopfner et al. (1999) Structure 7(8):989-96.

The term “FIX zymogen” can be used interchangeably herein with “FIXz,” “FIX precursor,” “unactivated FIX,” “non-activated FIX,” or “non-activated FIX precursor.” In one embodiment, FIX zymogen (FIXz) includes non-activated FIX precursor in which the activation peptide (e.g., 35 activation peptide that are represented as amino acids 146 to 180 of SEQ ID NO: 764 (mature numbering) is not cleaved from the precursor. FIX zymogen can include any naturally-occurring or engineered variants. A non-limiting example of FIX zymogen is shown in SEQ ID NO: 764. In another embodiment, FIX zymogen is non-activatable FIX (FIXn), which is engineered to be non-active in the presence of Factor XIa, activated plasma thromboplastin antecedent. An example of non-activatable FIX can be FIX carrying an arginine to alanine mutation at position 180 (mature numbering) preventing its activation and maintaining Factor IX in the zymogen form (FIXz). FIX zymogen can optionally contain a signal peptide and/or propeptide.

The term “activated FIX” can be used interchangeably herein with “FIXa”. In one embodiment, activated FIX is wild-type, naturally occurring FIXa (also referred to herein as “wild-type FIXa”). In another embodiment, FIXa comprises non-naturally occurring FIXa, e.g., FIXa conformational variant. For example, FIXa can be a FIXa-SM, which is designed to have the same conformation as wild-type, naturally occurring FIXa bound to its substrate, FX. In a particular embodiment, FIXa-SM is activated FIX with a substrate mimic covalently bound to the active site, which is intended to mimic the most active conformation of activated FIX.

FIX zymogen and FIXa can include FIX variants. In one embodiment, FIX variants have been cloned, as described in U.S. Pat. Nos. 4,770,999 and 7,700,734, and cDNA coding for human Factor IX has been isolated, characterized, and cloned into expression vectors (see, for example, Choo et al., Nature 299:178-180 (1982); Fair et al., Blood 64:194-204 (1984); and Kurachi et al., Proc. Natl. Acad. Sci., U.S.A. 79:6461-6464 (1982)). One particular variant of FIX, the R338L FIX (Padua) variant, characterized by Simioni et al, 2009, comprises a gain-of-function mutation, which correlates with a nearly 8-fold increase in the activity of the Padua variant relative to native FIX. FIX variants can also include any FIX polypeptide having one or more conservative amino acid substitutions, which do not affect the FIX activity of the FIX polypeptide.

The present disclosure therefore provides an antibody (e.g., an isolated antibody), or an antigen binding portion thereof, that specifically binds to activated factor IX (FIXa) (e.g., free FIXa or FIXa-SM), wherein the anti-FIXa antibody or antigen binding portion thereof preferentially binds to FIXa in the presence of FIXa and FIX zymogen (“anti-FIXa antibody or antigen binding portion thereof”).

In some aspects, the anti-FIXa antibody, or antigen binding portion thereof, binds to FIXa with a binding affinity higher than a binding affinity of the anti-FIXa antibody or antigen binding portion thereof to FIXz. In one embodiment the binding affinity is expressed as K_{D}.

The present disclosure also provides an isolated anti-FIXa antibody, or antigen binding portion thereof, which binds to FIXa with a binding affinity higher than a binding affinity of the anti-FIXa antibody or antigen binding portion thereof to FIXz. In some aspects, the anti-FIXa antibody, or antigen binding portion thereof, binds to FIXa with a K_{D} of about 100 nM or less (e.g., 1 nM to 100 nM or 0.1 nM to 100 nM), about 95 nM or less, about 90 nM or less, about 85 nM or less, about 80 nM or less, about 75 nM or less, about 70 nM or less, about 65 nM or less, about 60 nM or less, about 55 nM or less, about 50 nM or less, about 45 nM or less, about 40 nM or less, about 35 nM or less, about 30 nM or less, about 25 nM or less, about 20 nM or less, about 15 nM or less, about 10 nM or less, about 5 nM or less, or about 1 nM or less as determined by a Bio-Layer Interferometry (BLI) assay. In other embodiments, the anti-FIXa antibody, or antigen binding portion thereof, binds to FIXa with a K_{D} of about 10 nM or less, about 9 nM or less, about 8 nM or less, about 7 nM or less, about 6 nM or less, about 5 nM or less, about 4 nM or less, about 3 nM or less, about 2 nM or less, about 1 nM or less, about 0.5 nM or less, about 0.2 nM or less, about 0.1 nM or less, or about 0.05 nM or less. In yet other embodiments, the anti-FIXa antibody, or antigen binding portion thereof, binds to FIXa with a K_{D} of 1 nM to 100 nM, 1 nM to 90 nM, 1 nM to 80 nM, 1 nM to 70 nM, 1 nM to 60 nM, 1 nM to 50 nM, 1 nM to 40 nM, 1 nM to 30 nM, 1 nM to 20 nM, 1 nM to 10 nM, 0.1 nM to 100 nM, 0.1 nM to 90 nM, 0.1 nM to 80 nM, 0.1 nM to 70 nM, 0.1 nM to 60 nM, 0.1 nM to 50 nM, 0.1 nM to 40 nM, 0.1 nM to 30 nM, 0.1 nM to 20 nM, 0.1 nM to 10 nM, or 0.1 nM to 1 nM.

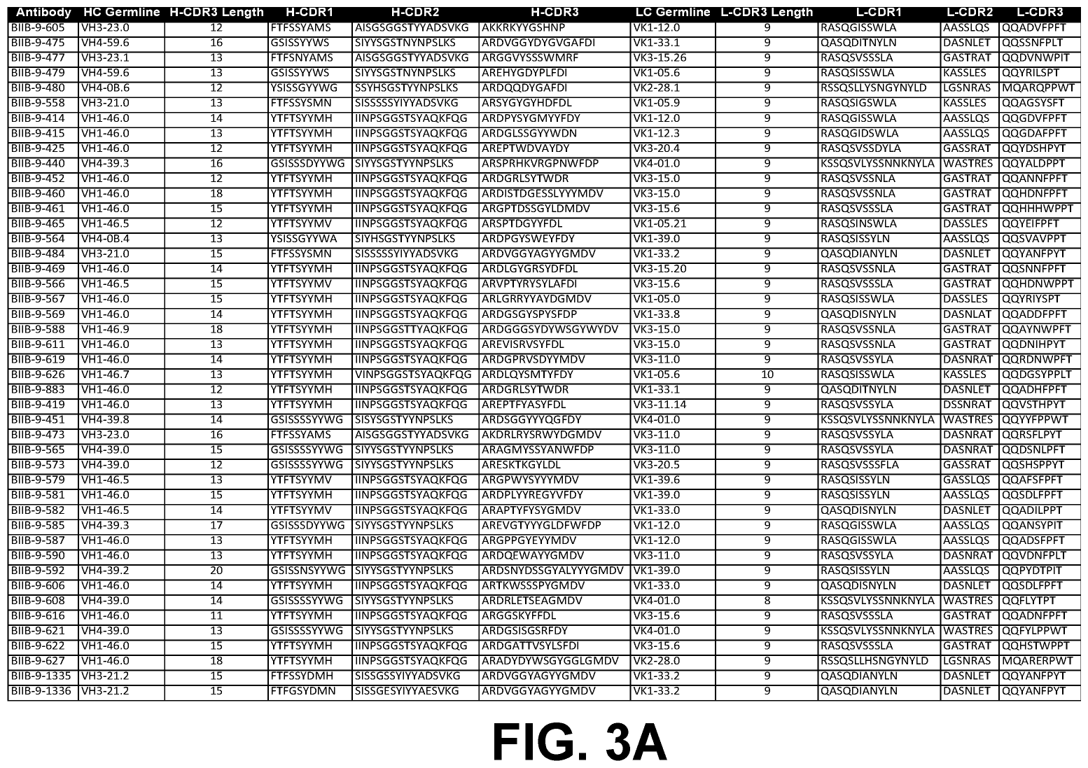

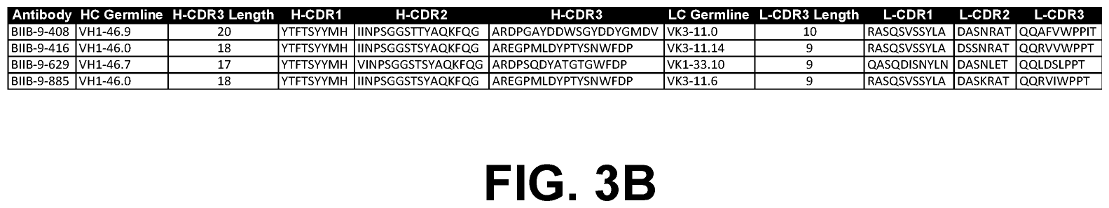

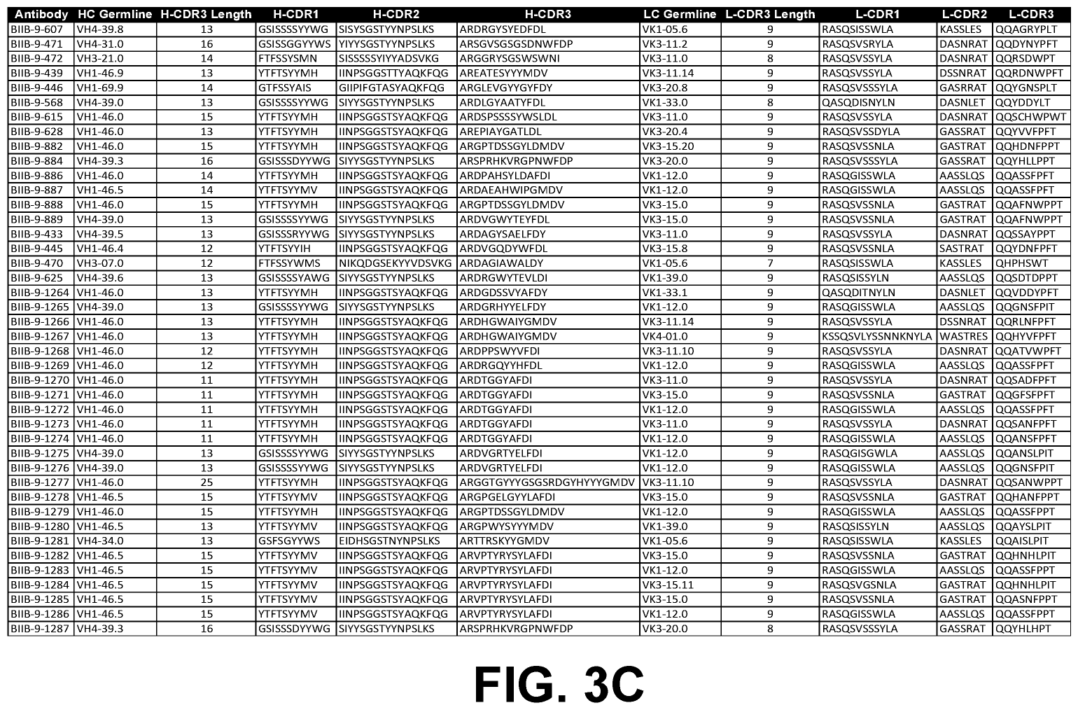

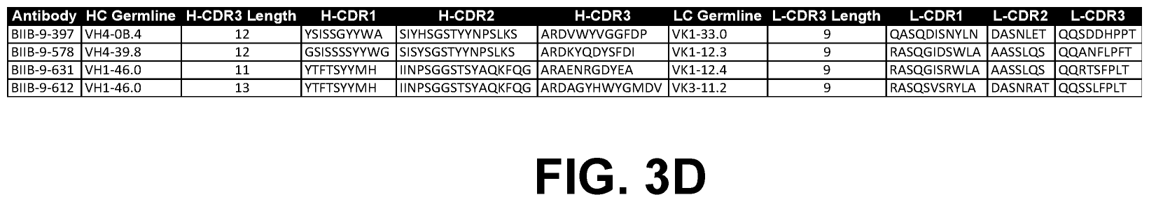

In some aspects, the anti-FIXa antibody, or antigen binding portion thereof, cross-competes with a reference antibody selected from the group consisting of the antibodies in FIGS. 3A, 3B, and/or 3C. In some aspects, the anti-FIXa antibody, or antigen binding portion thereof, binds to the same epitope as a reference antibody selected from the group consisting of the antibodies in FIGS. 3A, 3B, and/or 3C. In some aspects, the reference antibody is selected from BIIB-9-484, BIIB-9-440, BIIB-9-882, BIIB-9-460, BIIB-9-433, and any combination thereof.

In further aspects, the anti-FIXa antibody or antigen binding portion thereof can further be classified into three classes:

Class I: anti-FIXa antibodies or antigen binding portion thereof that preferentially binds to FIXa-SM over free FIXa or FIXz (FIG. 3A antibodies);
Class II: anti-FIXa antibodies or antigen binding portion thereof that preferentially binds to free FIXa over FIXa-SM or FIXz (FIG. 3B antibodies); and
Class III: anti-FIXa antibodies or antigen binding portion thereof that binds to free FIXa and FIXa-SM with near equivalency, but do not appreciably associate with FIXz (FIG. 3C antibodies).

In some embodiments, the anti-FIXa antibody, or antigen binding portion thereof, cross-competes with a reference antibody selected from the group consisting of the antibodies in FIG. 3A. In other embodiments, the anti-FIXa antibody, or antigen binding portion thereof, binds to the same epitope as a reference antibody selected from the group consisting of the antibodies in FIG. 3A. In some embodiments, the anti-FIXa antibody, or antigen binding portion thereof, cross-competes with a reference antibody selected from the group consisting of the antibodies in FIG. 3B. In other embodiments, the anti-FIXa antibody, or antigen binding portion thereof, binds to the same epitope as a reference antibody selected from the group consisting of the antibodies in FIG. 3B. In some embodiments, the anti-FIXa antibody, or antigen binding portion thereof, cross-competes with a reference antibody selected from the group consisting of the antibodies in FIG. 3C. In other embodiments, the anti-FIXa antibody, or antigen binding portion thereof, binds to the same epitope as a reference antibody selected from the group consisting of the antibodies in FIG. 3C.

In some aspects, the anti-FIXa antibody, or antigen binding portion thereof, comprises a VH CDR1, a VH CDR2, and a VH CDR3, wherein the VH CDR3 comprises
(i) a VH CDR3 sequence identical to a VH CDR3 sequence selected from the group consisting of VH CDR3 sequences in FIG. 3A, or
(ii) a VH CDR3 sequence identical to a VH CDR3 sequence selected from the group consisting of VH CDR3 sequences in FIG. 3A except for 1, 2, or 3 amino acid substitutions.

In other aspects, the anti-FIXa antibody, or antigen binding portion thereof, comprises a VH CDR1, a VH CDR2, and a VH CDR3, wherein the VH CDR3 comprises
(i) a VH CDR3 sequence identical to a VH CDR3 sequence selected from the group consisting of VH CDR3 sequences in FIG. 3B, or
(ii) a VH CDR3 sequence identical to a VH CDR3 sequence selected from the group consisting of VH CDR3 sequences in FIG. 3B except for 1, 2, or 3 amino acid substitutions.

In some aspects, the anti-FIXa antibody, or antigen binding portion thereof, comprises a VH CDR1, a VH CDR2, and a VH CDR3, wherein the VH CDR3 comprises
(i) a VH CDR3 sequence identical to a VH CDR3 sequence selected from the group consisting of VH CDR3 sequences in FIG. 3C, or
(ii) a VH CDR3 sequence identical to a VH CDR3 sequence selected from the group consisting of VH CDR3 sequences in FIG. 3C except for 1, 2, or 3 amino acid substitutions.

In some aspects, the amino acid substitutions are conservative amino acid substitutions. In other aspects, the amino acid substitutions are back mutation.

In some aspects, the anti-FIXa antibody, or antigen binding portion thereof, comprises a VH CDR1, VH CDR2, and VH CDR3, wherein the VH CDR3 sequence comprises the amino acid sequence ARDX₁X₂X₃X₄X₅X₆YYX₇MDV (SEQ ID NO:753), wherein X₁ is V or G, X₂ is G or V, X₃ is G or R, X₄ is Y or V, X₅ is A or S, X₆ is G or D, X₇ is G or none.

A person of skill in the art would understand that when a position is described as “none” or “absent” in a consensus sequence, such absence does not indicate a breakage in the polypeptide chain. These terms merely reflects the occurrence of insertions and deletions in the amino acid chains as observed in a multiple sequence alignment. Thus, when two sequences, one of which contains an amino acid insertion, are aligned to generate a consensus sequence, the sequence lacking the insertion will have an “absent” (“none”) amino acid at that position.

In some aspects, the anti-FIXa antibody, or antigen binding portion thereof, comprises a VH CDR1, a VH CDR2, and a VH CDR3, wherein the VH CDR3 sequence comprises an amino acid sequence selected from ARDVGGYAGYYGMDV (SEQ ID NO: 905; VH CDR 3 for BIIB-9-484; BIIB-9-1335; and BIIB-9-1336), ARDISTDGESSLYYYMDV (SEQ ID NO: 901; BIIB-9-460), ARGPTDSSGYLDMDV (SEQ ID NO: 1186; BIIB-9-882), ARSPRHKVRGPNWFDP (SEQ ID NO: 899; BIIB-9-440), or ARDGPRVSDYYMDV (SEQ ID NO: 912; BIIB-9-619). In some aspects, the VH CDR3 sequences disclosed herein can comprise 1, 2, or 3 amino acid substitutions.

In some aspects, the anti-FIXa antibody, or antigen binding portion thereof, comprises a VH CDR1, a VH CDR2, and VH CDR3, wherein the VH CDR1 sequence comprises
(i) a VH CDR1 sequence identical to a sequence selected from the group consisting of the VH CDR1 sequences disclosed in FIG. 3A, or
(ii) a VH CDR1 sequence identical to a sequence selected from the group consisting of the VH CDR1 sequences disclosed in FIG. 3A except for 1, 2, or 3 amino acid substitutions.

In some aspects, the anti-FIXa antibody, or antigen binding portion thereof, comprises a VH CDR1, a VH CDR2, and VH CDR3, wherein the VH CDR1 sequence comprises
(i) a VH CDR1 sequence identical to a sequence selected from the group consisting of the VH CDR1 sequences disclosed in FIG. 3B, or
(ii) a VH CDR1 sequence identical to a sequence selected from the group consisting of the VH CDR1 sequences disclosed in FIG. 3B except for 1, 2, or 3 amino acid substitutions.

In some aspects, the anti-FIXa antibody, or antigen binding portion thereof, comprises a VH CDR1, a VH CDR2, and VH CDR3, wherein the VH CDR1 sequence comprises
(i) a VH CDR1 sequence identical to a sequence selected from the group consisting of the VH CDR1 sequences disclosed in FIG. 3C, or
(ii) a VH CDR1 sequence identical to a sequence selected from the group consisting of the VH CDR1 sequences disclosed in FIG. 3C except for 1, 2, or 3 amino acid substitutions.

In some aspects, the anti-FIXa antibody, or antigen binding portion thereof, comprises a VH CDR1, a VH CDR2, and VH CDR3, wherein the VH CDR2 sequence comprises
(i) a VH CDR2 sequence identical to a sequence selected from the group consisting of the VH CDR2 sequences disclosed in FIG. 3A, or
(ii) a VH CDR2 sequence identical to a sequence selected from the group consisting of the VH CDR2 sequences disclosed in FIG. 3A except for 1, 2, or 3 amino acid substitutions.

In some aspects, the anti-FIXa antibody, or antigen binding portion thereof, comprises a VH CDR1, a VH CDR2, and VH CDR3, wherein the VH CDR2 sequence comprises
(i) a VH CDR2 sequence identical to a sequence selected from the group consisting of the VH CDR2 sequences disclosed in FIG. 3B, or
(ii) a VH CDR2 sequence identical to a sequence selected from the group consisting of the VH CDR2 sequences disclosed in FIG. 3B except for 1, 2, or 3 amino acid substitutions.

In some aspects, the anti-FIXa antibody, or antigen binding portion thereof, comprises a VH CDR1, a VH CDR2, and VH CDR3, wherein the VH CDR2 sequence comprises
(i) a VH CDR2 sequence identical to a sequence selected from the group consisting of the VH CDR2 sequences disclosed in FIG. 3C, or
(ii) a VH CDR2 sequence identical to a sequence selected from the group consisting of the VH CDR2 sequences disclosed in FIG. 3C except for 1, 2, or 3 amino acid substitutions.

In some aspects, the anti-FIXa antibody, or antigen binding portion thereof, comprises a VL CDR1, a VL CDR2, and a VL CDR3, wherein the VL CDR1 sequence comprises
(i) a VL CDR1 sequence identical to a sequence selected from the group consisting of the VL CDR1 sequences disclosed in FIG. 3A, or
(ii) a VL CDR1 sequence identical to a sequence selected from the group consisting of the VL CDR1 sequences disclosed in FIG. 3A except for 1, 2, or 3 amino acid substitutions.

In some aspects, the anti-FIXa antibody, or antigen binding portion thereof, comprises a VL CDR1, a VL CDR2, and a VL CDR3, wherein the VL CDR1 sequence comprises
(i) a VL CDR1 sequence identical to a sequence selected from the group consisting of the VL CDR1 sequences disclosed in FIG. 3B, or
(ii) a VL CDR1 sequence identical to a sequence selected from the group consisting of the VL CDR1 sequences disclosed in FIG. 3B except for 1, 2, or 3 amino acid substitutions.

In some aspects, the anti-FIXa antibody, or antigen binding portion thereof, comprises a VL CDR1, a VL CDR2, and a VL CDR3, wherein the VL CDR1 sequence comprises
(i) a VL CDR1 sequence identical to a sequence selected from the group consisting of the VL CDR1 sequences disclosed in FIG. 3C, or
(ii) a VL CDR1 sequence identical to a sequence selected from the group consisting of the VL CDR1 sequences disclosed in FIG. 3C except for 1, 2, or 3 amino acid substitutions.

In some aspects, the anti-FIXa antibody, or antigen binding portion thereof, comprises a VL CDR1, a VL CDR2, and a VL CDR3, wherein the VL CDR2 sequence comprises
(i) a VL CDR2 sequence identical to a sequence selected from the group consisting of the VL CDR2 sequences in FIG. 3A, or
(ii) a VL CDR2 sequence identical to a sequence selected from the group consisting of the VL CDR2 sequences in FIG. 3A except for 1, 2, or 3 amino acid substitutions.

In some aspects, the anti-FIXa antibody, or antigen binding portion thereof, comprises a VL CDR1, a VL CDR2, and a VL CDR3, wherein the VL CDR2 sequence comprises
(i) a VL CDR2 sequence identical to a sequence selected from the group consisting of the VL CDR2 sequences in FIG. 3B, or
(ii) a VL CDR2 sequence identical to a sequence selected from the group consisting of the VL CDR2 sequences in FIG. 3B except for 1, 2, or 3 amino acid substitutions.

In some aspects, the anti-FIXa antibody, or antigen binding portion thereof, comprises a VL CDR1, a VL CDR2, and a VL CDR3, wherein the VL CDR2 sequence comprises
(i) a VL CDR2 sequence identical to a sequence selected from the group consisting of the VL CDR2s sequences in FIG. 3C, or
(ii) a VL CDR2 sequence identical to a sequence selected from the group consisting of the VL CDR2s sequences in FIG. 3C except for 1, 2, or 3 amino acid substitutions.

In some aspects, the anti-FIXa antibody, or antigen binding portion thereof, comprises a VL CDR1, a VL CDR2, and a VL CDR3, wherein the VL CDR3 sequence comprises
(i) a VL CDR3 sequence identical to a sequence selected from the group consisting of the VL CDR3 sequences disclosed in FIG. 3A, or
(ii) a VL CDR3 sequence identical to a sequence selected from the group consisting of the VL CDR3 sequences disclosed in FIG. 3A except for 1, 2, or 3 amino acid substitutions.

In some aspects, the anti-FIXa antibody, or antigen binding portion thereof, comprises a VL CDR1, a VL CDR2, and a VL CDR3, wherein the VL CDR3 sequence comprises
(i) a VL CDR3 sequence identical to a sequence selected from the group consisting of the VL CDR3 sequences disclosed in FIG. 3B, or
(ii) a VL CDR3 sequence identical to a sequence selected from the group consisting of the VL CDR3 sequences disclosed in FIG. 3B except for 1, 2, or 3 amino acid substitutions.

In some aspects, the anti-FIXa antibody, or antigen binding portion thereof, comprises a VL CDR1, a VL CDR2, and a VL CDR3, wherein the VL CDR3 sequence comprises
(i) a VL CDR3 sequence identical to a sequence selected from the group consisting of the VL CDR3 sequences disclosed in FIG. 3C, or
(ii) a VL CDR3 sequence identical to a sequence selected from the group consisting of the VL CDR3 sequences disclosed in FIG. 3C except for 1, 2, or 3 amino acid substitutions.

The present disclosure also provides an isolated antibody, or antigen binding portion thereof, which specifically binds to FIXa, comprising a VH CDR1, a VH CDR2, and a VH CDR3, and a VL CDR1, a VL CDR2, and a VL CDR3, wherein the VH CDR1, VH CDR2, and VH CDR3 and the VL CDR1, VL CDR2, and VL CDR3 comprise the VH CDR1, VH CDR2, and VH CDR3 and the VL CDR1, VL CDR2, and VL CDR3 of an anti-FIXa antibody selected from the group consisting of the antibodies in FIG. 3A: BIIB-9-605, BIIB-9-475, BIIB-9-477, BIIB-9-479, BIIB-9-480, BIIB-9-558, BIIB-9-414, BIIB-9-415, BIIB-9-425, BIIB-9-440, BIIB-9-452, BIIB-9-460, BIIB-9-461, BIIB-9-465, BIIB-9-564, BIIB-9-484, BIIB-9-469, BIIB-9-566, BIIB-9-567, BIIB-9-569, BIIB-9-588, BIIB-9-611, BIIB-9-619, BIIB-9-626, BIIB-9-883, BIIB-9-419, BIIB-9-451, BIIB-9-473, BIIB-9-565, BIIB-9-573, BIIB-9-579, BIIB-9-581, BIIB-9-582, BIIB-9-585, BIIB-9-587, BIIB-9-590, BIIB-9-592, BIIB-9-606, BIIB-9-608 BIIB-9-616, BIIB-9-621, BIIB-9-622, BIIB-9-627, BIIB-9-1335, and BIIB-9-1336.

In some embodiments, the disclosure includes an isolated antibody, or antigen binding portion thereof, which specifically binds to FIXa, comprising a VH CDR1, a VH CDR2, and a VH CDR3, and a VL CDR1, a VL CDR2, and a VL CDR3, wherein the VH CDR1, VH CDR2, and VH CDR3 and the VL CDR1, VL CDR2, and VL CDR3 comprise the VH CDR1, VH CDR2, and VH CDR3 and the VL CDR1, VL CDR2, and VL CDR3 of an anti-FIXa antibody selected from the group consisting of the antibodies in FIG. 3B: BIIB-9-408, BIIB-9-416, BIIB-9-629, or BIIB-9-885.

In other embodiments, the disclosure provides an isolated antibody, or antigen binding portion thereof, which specifically binds to FIXa, comprising a VH CDR1, a VH CDR2, and a VH CDR3, and a VL CDR1, a VL CDR2, and a VL CDR3, wherein the VH CDR1, VH CDR2, and VH CDR3 and the VL CDR1, VL CDR2, and VL CDR3 comprise the VH CDR1, VH CDR2, and VH CDR3 and the VL CDR1, VL CDR2, and VL CDR3 of an anti-FIXa antibody selected from the group consisting of the antibodies in FIG. 3C: BIIB-9-607, BIIB-9-471, BIIB-9-472, BIIB-9-439, BIIB-9-446, BIIB-9-568, BIIB-9-615, BIIB-9-628, BIIB-9-882, BIIB-9-884, BIIB-9-886, BIIB-9-887, BIIB-9-888, BIIB-9-889, BIIB-9-433, BIIB-9-445, BIIB-9-470, BIIB-9-625, BIIB-9-1264, BIIB-9-1265, BIIB-9-1266, BIIB-9-1267, BIIB-9-1268, BIIB-9-1269, BIIB-9-1270, BIIB-9-1271, BIIB-9-1272, BIIB-9-1273, BIIB-9-1274, BIIB-9-1275, BIIB-9-1276, BIIB-9-1277, BIIB-9-1278, BIIB-9-1279, BIIB-9-1280, BIIB-9-1281, BIIB-9-1282, BIIB-9-1283, BIIB-9-1284, BIIB-9-1285, BIIB-9-1286, and BIIB-9-1287.

In some aspects, the anti-FIXa antibody, or antigen binding portion thereof, comprises VH CDR1, VH CDR2, and VH CDR3 sequences comprising SEQ ID NOs: 800-844, SEQ ID NOs: 845-889, and SEQ ID NOs: 890-934, respectively (VH CDRs for Class I antibodies), and/or VL CDR1, VL CDR2, and VL CDR3 sequences comprising SEQ ID NOs: 935-979, SEQ ID NOs: 980-1024, and SEQ ID NO: 1025-1069, respectively (VL CDRs for Class I antibodies).

In some aspects, the anti-FIXa antibody, or antigen binding portion thereof, comprises VH CDR1, VH CDR2, and VH CDR3 sequences comprising SEQ ID NOs: 1070-1073, SEQ ID NOs: 1074-1077, and SEQ ID NOs: 1078-1081, respectively (VH CDRs for Class II antibodies), and/or VL CDR1, VL CDR2, and VL CDR3 sequences comprising SEQ ID NOs: 1082-1085, SEQ ID NOs: 1086-1089, and SEQ ID NO: 1090-1093, respectively (VL CDRs for Class II antibodies).

In some aspects, the anti-FIXa antibody, or antigen binding portion thereof, comprises VH CDR1, VH CDR2, and VH CDR3 sequences comprising SEQ ID NOs: 1094-1135, SEQ ID NOs: 1136-1177, and SEQ ID NOs: 1178-1219, respectively (VH CDRs for Class III antibodies), and/or VL CDR1, VL CDR2, and VL CDR3 sequences comprising SEQ ID NOs: 1220-1261, SEQ ID NOs: 1262-1303, and SEQ ID NO: 1304-1345, respectively (VL CDRs for Class III antibodies).

In some aspects, the anti-FIXa antibody, or antigen binding portion thereof, comprises VH CDR1, VH CDR2, and VH CDR3 sequences comprising SEQ ID NO: 815, SEQ ID NO: 860, and SEQ ID NO: 905, respectively (VH CDRs for BIIB-9-484 antibody), and/or VL CDR1, VL CDR2, and VL CDR3 sequences comprising SEQ ID NO: 950, SEQ ID NO: 995, and SEQ ID NO: 1040, respectively (VL CDRs for BIIB-9-484 antibody).

In some aspects, the anti-FIXa antibody, or antigen binding portion thereof, comprises VH CDR1, VH CDR2, and VH CDR3 sequences comprising SEQ ID NO: 843, SEQ ID NO: 888, and SEQ ID NO: 933, respectively (VH CDRs for BIIB-9-1335 antibody), and/or VL CDR1, VL CDR2, and VL CDR3 sequences comprising SEQ ID NO: 950, SEQ ID NO: 995, and SEQ ID NO: 1040, respectively (VL CDRs for BIIB-9-1335 antibody).

In some aspects, the anti-FIXa antibody, or antigen binding portion thereof, comprises VH CDR1, VH CDR2, and VH CDR3 sequences comprising SEQ ID NO: 844, SEQ ID NO: 889, and SEQ ID NO: 934, respectively (VH CDRs for BIIB-9-1336 antibody), and/or VL CDR1, VL CDR2, and VL CDR3 sequences comprising SEQ ID NO: 950, SEQ ID NO: 995, and SEQ ID NO: 1040, respectively (VL CDRs for BIIB-9-1336 antibody).

In other aspects, the anti-FIXa antibody, or antigen binding portion thereof, cross-competes with antibodies BIIB-9-484, BIIB-9-1335, and BIIB-9-1336 and/or binds to the same epitope as antibodies BIIB-9-484, BIIB-9-1335, and BIIB-9-1336. In certain aspects, the anti-FIXa antibody, or antigen binding portion thereof, comprises VH CDR1, VH CDR2, and VH CDR3 and VL CDR1, VL CDR2, and VL CDR3, wherein the VH CDR3 comprises ARDVGGYAGYYGMDV (SEQ ID NO: 905, BIIB-9-484 VH CDR3), VH CDR2 comprises SISSX₁X₂SYIYYAX₃SVKG (SEQ ID NO: 754), wherein X₁ is S, G, or any conservative substitution, X₂ is S, E, or any conservative substitution, and X₃ is D, E, or any conservative substitution, VH CDR1 comprises FTFX₄SYX₅MX₆ (SEQ ID NO: 755), wherein X₄ is S, G, or any conservative substitution, X₅ is D, S, or any conservative substitution, and X₆ is H, N, or any conservative substitution. The anti-FIXa antibody, or antigen binding portion thereof, can comprises SEQ ID NO: 815 for VH CDR1, SEQ ID NO: 860 for VH CDR2, and SEQ ID NO: 905 for VH CDR3. (BIIB-9-484 VH CDRs)

In some aspects, the anti-FIXa antibody, or antigen binding portion thereof, comprises

(a1) VH CDR1, VH CDR2, and VH CDR3 sequences comprising SEQ ID NO: 809, SEQ ID NO: 854, and SEQ ID NO: 899, respectively (VH CDRs for BIIB-9-440 antibody), and/or VL CDR1, VL CDR2, and VL CDR3 sequences comprising SEQ ID NO: 944, SEQ ID NO: 989, and SEQ ID NO: 1034, respectively (VL CDRs for BIIB-9-440 antibody);

(a2) VH CDR1, VH CDR2, and VH CDR3 sequences comprising SEQ ID NO: 1102, SEQ ID NO: 1144, and SEQ ID NO: 1186, respectively (VH CDRs for BIIB-9-882 antibody), and/or VL CDR1, VL CDR2, and VL CDR3 sequences comprising SEQ ID NO: 1228, SEQ ID NO: 1270, and SEQ ID NO: 1312, respectively (VL CDRs for BIIB-9-882 antibody);

(a3) VH CDR1, VH CDR2, and VH CDR3 sequences comprising SEQ ID NO: 811, SEQ ID NO: 856, and SEQ ID NO: 901, respectively (VH CDRs for BIIB-9-460 antibody), and/or VL CDR1, VL CDR2, and VL CDR3 sequences comprising SEQ ID NO: 946, SEQ ID NO: 991, and SEQ ID NO: 1036, respectively (VL CDRs for BIIB-9-460 antibody); or,

(a4) VH CDR1, VH CDR2, and VH CDR3 sequences comprising SEQ ID NO: 1108, SEQ ID NO: 1150, and SEQ ID NO: 1192, respectively (VH CDRs for BIIB-9-433 antibody), and/or VL CDR1, VL CDR2, and VL CDR3 sequences comprising SEQ ID NO: 1234, SEQ ID NO: 1276, and SEQ ID NO: 1318, respectively (VL CDRs for BIIB-9-433 antibody);

In some aspects, the anti-FIXa antibody, or antigen binding portion thereof, comprises VH CDR1, VH CDR2, and VH CDR3 sequences comprising SEQ ID NO: 822, SEQ ID NO: 867, and SEQ ID NO: 912, respectively (VH CDRs for BIIB-9-619 antibody), and/or VL CDR1, VL CDR2, and VL CDR3 sequences comprising SEQ ID NO: 957, SEQ ID NO: 1002, and SEQ ID NO: 1047, respectively (VL CDRs for BIIB-9-619 antibody).

In some aspects, the anti-FIXa antibody, or antigen binding portion thereof, comprises

(i) VH CDR1, VH CDR2, and VH CDR3 sequences comprising SEQ ID NO: 843, SEQ ID NO: 888, and SEQ ID NO: 933, respectively (VH CDRs for BIIB-9-1335 antibody), and/or VL CDR1, VL CDR2, and VL CDR3 sequences comprising SEQ ID NO: 950, SEQ ID NO: 995, and SEQ ID NO: 1040, respectively, respectively (VL CDRs for BIIB-9-1335 antibody); or,
(ii) VH CDR1, VH CDR2, and VH CDR3 sequences comprising SEQ ID NO: 844, SEQ ID NO: 889, and SEQ ID NO: 934, respectively (VH CDRs for BIIB-9-1336 antibody), and/or VL CDR1, VL CDR2, and VL CDR3 sequences comprising SEQ ID NO: 950, SEQ ID NO: 995, and SEQ ID NO: 1040, respectively (VL CDRs for BIIB-9-1336 antibody).

In some aspects, the anti-FIXa antibody, or antigen binding portion thereof, comprises a VH, wherein the VH comprises an amino acid sequence which is at least about 70%, at least about 75%, at least about 80%, at least about 85%, at least about 90%, at least about 95%, at least about 96%, at least about 97%, at least about 98%, at least about 99%, or about 100% identical to an amino acid sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, and 181 (SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, and 89 for Class I antibodies; SEQ ID NOs: 91, 93, 95, and 97 for Class II antibodies; and SEQ ID NOs: 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, and 181 for Class III antibodies).

In some aspects, the anti-FIXa antibody, or antigen binding portion thereof, comprises a VL, wherein the VL comprises an amino acid sequence which is at least about 70%, at least about 75%, at least about 80%, at least about 85%, at least about 90%, at least about 95%, at least about 96%, at least about 97%, at least about 98%, at least about 99%, or about 100% identical an the amino acid sequence selected from the group consisting of SEQ ID NOs: 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, and 367 (SEQ ID NOs: 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, and 275 for Class I antibodies; SEQ ID NOs: 277, 279, 281, and 283 for Class II antibodies; SEQ ID NOs: 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, and 367 for Class III antibodies).

In some aspects, the anti-FIXa antibody, or antigen binding portion thereof, comprises a VH and a VL, wherein

(i) the VH comprises an amino acid sequence which is at least about 70%, at least about 75%, at least about 80%, at least about 85%, at least about 90%, at least about 95%, at least about 96%, at least about 97%, at least about 98%, at least about 99%, or about 100% identical to an amino acid sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, and 181 (SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, and 89 for Class I antibodies; SEQ ID NOs: 91, 93, 95, and 97 for Class II antibodies; and SEQ ID NOs: 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, and 181 for Class III antibodies); and,
(ii) the VL comprises an amino acid sequence which is at least about 70%, at least about 75%, at least about 80%, at least about 85%, at least about 90%, at least about 95%, at least about 96%, at least about 97%, at least about 98%, at least about 99%, or about 100% identical to an amino acid sequence selected from the group consisting of SEQ ID NOs: 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, and 367 (SEQ ID NOs: 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, and 275 for Class I antibodies; SEQ ID NOs: 277, 279, 281, and 283 for Class II antibodies; SEQ ID NOs: 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, and 367 for Class III antibodies).

In certain aspects, an anti-FIXa antibody comprises a heavy chain variable region from a particular germline heavy chain immunoglobulin gene and/or a light chain variable region from a particular germline light chain immunoglobulin gene. In some embodiments, the VH sequence of the anti-FIXa antibody can be derived from any one of V, D, or J germline sequences and/or the VL sequence of the anti-FIXa antibody can be derived from any one of kappa or lambda germline sequence.

As demonstrated herein, human antibodies specific for FIXa have been prepared that comprise a heavy chain variable region that is the product of or derived from a human germline gene. Accordingly, provided herein are isolated FIXa antibodies, or antigen-binding portions thereof, comprising a heavy chain variable region that is the product of or derived from a human VH germline gene selected from the group consisting of: VH1-18, VH1-46, VH3-21, VH3-30, VH4-31, VH4-39, VH4-0B, VH5-51, and any combination thereof. In particular embodiments, the VH germline gene is selected from the group consisting of VH1-18.0, VH1-18.1, VH1-18.8, VH1-46.0, VH1-46.4, VH1-46.5, VH1-46.6, VH1-46.7, VH1-46.8, VH1-46.9, VH3-21.0, VH3-23.0, VH3-23.2, VH3-23.6, VH3-30.0, VH4-31.5, VH4-39.0, VH4-39.5. VH4-0B.4, VH5-51.1, and any combination thereof.

In other aspects, provided herein are isolated FIXa antibodies, or antigen-binding portions thereof, comprising a heavy chain variable region that is the product of or derived from a human VL germline gene selected from the group consisting of: VK1-05, VK1-12, VK1-39, VK2-28, VK3-11, VK3-15, VK3-20, VK4-01, and any combination thereof. In particular embodiments, the VL germline gene is selected from the group consisting of VK1-05.6, VK1-05.12, VK1-12.0, VK1-12.4, VK1-12.7, VK1-12.10, VK1-12.15, VK1-39.0, VK1-39.3, VK1-39.15, VK2-28.0, VK2-28.1, VK2-28.5, VK3-11.0, VK3-11.2, VK3-11.6, VK3-11.14, VK3-15.0, VK3-15.8, VK3-15.10, VK3-20.0, VK3-20.1, VK3-20.4, VK3-20.5, VK4-01.0, VK4-01.4, VK4-01.20, and any combination thereof.

Antibodies described herein include those comprising a heavy chain variable region that is the product of or derived from one of the above-listed human germline VH genes and also comprising a light chain variable region that is the product of or derived from one of the above-listed human germline VK genes, as shown in the Figures.

As used herein, a human antibody comprises heavy and light chain variable regions that are “the product of or “derived from” a particular germline sequence if the variable regions of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody that is “the product of or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “the product of” or “derived from” a particular human germline immunoglobulin sequence can contain amino acid differences as compared to the germline sequence, due to, for example, naturally-occurring somatic mutations or intentional introduction of site-directed mutation. However, a selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody can be at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody can display no more than 5, or even no more than 4, 3, 2, or 1 amino acid difference from the amino acid sequence encoded by the germline immunoglobulin gene.

In some aspects, the anti-FIXa antibody, or antigen binding portion thereof, comprises a VH and a VL, wherein

(a1) VH comprises an amino acid sequence at least 70%, at least 80%, at least 90%, at least 95%, at least 96%, at least 97%, at least 98%, at least 99%, or 100% identical to SEQ ID NO: 31 and VL comprises an amino acid sequence at least 70%, at least 80%, at least 90%, at least 95%, at least 96%, at least 97%, at least 98%, at least 99%, or 100% identical to SEQ ID NO: 221 (VH and VL of BIIB-9-484, respectively);
(a2) VH comprises an amino acid sequence at least 70%, at least 80%, at least 90%, at least 95%, at least 96%, at least 97%, at least 98%, at least 99%, or 100% identical to SEQ ID NO: 19 and VL comprises an amino acid sequence at least 70%, at least 80%, at least 90%, at least 95%, at least 96%, at least 97%, at least 98%, at least 99%, or 100% identical to SEQ ID 209 (VH and VL of BIIB-9-440, respectively);
(a3) VH comprises an amino acid sequence at least 70%, at least 80%, at least 90%, at least 95%, at least 96%, at least 97%, at least 98%, at least 99%, or 100% identical to SEQ ID NO: 115 and VL comprises an amino acid sequence at least 70%, at least 80%, at least 90%, at least 95%, at least 96%, at least 97%, at least 98%, at least 99%, or 100% identical to SEQ ID NO: 301 (VH and VL of BIIB-9-882, respectively);
(a4) VH comprises an amino acid sequence at least 70%, at least 80%, at least 90%, at least 95%, at least 96%, at least 97%, at least 98%, at least 99%, or 100% identical to SEQ ID NO: 23 and VL comprises an amino acid sequence at least 70%, at least 80%, at least 90%, at least 95%, at least 96%, at least 97%, at least 98%, at least 99%, or 100% identical to SEQ ID NO: 213 (VH and VL of BIIB-9-460, respectively);
(a5) VH comprises an amino acid sequence at least 70%, at least 80%, at least 90%, at least 95%, at least 96%, at least 97%, at least 98%, at least 99%, or 100% identical to SEQ ID NO: 127 and VL comprises an amino acid sequence at least 70%, at least 80%, at least 90%, at least 95%, at least 96%, at least 97%, at least 98%, at least 99%, or 100% identical to SEQ ID NO: 313 (VH and VL of BIIB-9-433, respectively);
(a6) VH comprises an amino acid sequence at least 70%, at least 80%, at least 90%, at least 95%, at least 96%, at least 97%, at least 98%, at least 99%, or 100% identical to SEQ ID NO: 45 and VL comprises an amino acid sequence at least 70%, at least 80%, at least 90%, at least 95%, at least 96%, at least 97%, at least 98%, at least 99%, or 100% identical to SEQ ID NO: 235 (VH and VL of BIIB-9-619, respectively);
(a7) VH comprises an amino acid sequence at least 70%, at least 80%, at least 90%, at least 95%, at least 96%, at least 97%, at least 98%, at least 99%, or 100% identical to SEQ ID NO: 87 and VL comprises an amino acid sequence at least 70%, at least 80%, at least 90%, at least 95%, at least 96%, at least 97%, at least 98%, at least 99%, or 100% identical to SEQ ID NO: 221 (VH and VL of BIIB-9-1335, respectively); or
(a8) VH comprises an amino acid sequence at least 70%, at least 80%, at least 90%, at least 95%, at least 96%, at least 97%, at least 98%, at least 99%, or 100% identical to SEQ ID NO: 89 and VL comprises an amino acid sequence at least 70%, at least 80%, at least 90%, at least 95%, at least 96%, at least 97%, at least 98%, at least 99%, or 100% identical to SEQ ID NO: 221 (VH and VL of BIIB-9-1336, respectively).

In some aspects, the anti-FIXa antibody, or antigen binding portion thereof, binds to the same epitope as BIIB-9-1336. In other aspects, the anti-FIXa antibody, or antigen binding portion thereof, binds to an epitope overlapping BIIB-9-1336's epitope. In some embodiments, the anti-FIXa antibody, or antigen binding portion thereof, binds to an epitope region comprising at least one amino acid located between chymotrypsinogen numbering positions 91 and 101, 125 and 128, 165 and 179, or 232 and 241 in the sequence of the heavy chain of FIXa (corresponding to positions 76 to 88, 112 to 115, 153 to 167, and 222 to 231, respectively in SEQ ID NO: 758).

As used herein, the phrase “chymotrypsinogen numbering amino acid residue” and grammatical variants thereof refers to the description of certain amino acids in FIX by homology to the serine protease chymotrypsinogen. In the present disclosure the chymotrypsinogen numbering within the serine protease domain was used according to Hopfner et al. (EMBO J. 1997; 16:6626-35). For the present disclosure the chymotrypsinogen numbering is only used when explicitly indicated herein. The correspondence between the disclosed chymotripsinogen numbering amino acid residues and the amino acid position is SEQ ID NO: 758 is provided in the table below:

Correspondence between FIXa chymotrypsinogen
numbering and SEQ ID NO: 758 positions

| FIXa chymotrypsinogen numbering | SEQ ID NO: 758 |
|---|---|
| amino acid residue | amino acid residue |
| H91 | H76 |
| H92 | H77 |
| N93 | N78 |
| H101 | H88 |
| D125 | D112 |
| K126 | K113 |
| E127 | E114 |
| Y128 | Y115 |
| R165 | R153 |
| Y177 | Y165 |
| N178 | N166 |
| N179 | N167 |
| S232 | S222 |
| R233 | R223 |
| Y234 | Y224 |
| V235 | V225 |
| N236 | N226 |
| W237 | W227 |
| E240 | E230 |
| K241 | K231 |
| N100 | N87 |
| K132 | K121 |
| Y137 | Y126 |
| R170 | R158 |
| T172 | T160 |
| F174 | F162 |
| T175 | T163 |
| H185 | H174 |
| E202 | E192 |
| G205 | G195 |

In some aspects, the anti-FIXa antibody, or antigen binding portion thereof, binds to an epitope comprising at least one of chymotrypsinogen numbering amino acid residues H91, H92, N93, H101, D125, K126, E127, Y128, R165, Y177, N178, N179, S232, R233, Y234, V235, N236, W237, E240, and K241 in the sequence of the heavy chain of FIXa (corresponding to positions H76, H77, N78, H88, D112, K113, E114, Y115, R153, Y165, N166, N167, S222, R223, Y224, V225, N226, W227, E230, and K231, respectively in SEQ ID NO: 758). In some aspects, the anti-FIXa antibody, or antigen binding portion thereof, binds to an epitope comprising at least 2, 3, 4, 5, 6, 7, 8, 9, 10, 11, 12, 13, 14, 15, 16, 17, 18, or 19 amino acid residues selected from the group consisting chymotrypsinogen numbering amino acid residues H91, H92, N93, H101, D125, K126, E127, Y128, R165, Y177, N178, N179, S232, R233, Y234, V235, N236, W237, E240, and K241 in the sequence of the heavy chain of FIXa (corresponding to positions H76, H77, N78, H88, D112, K113, E114, Y115, R153, Y165, N166, N167, S222, R223, Y224, V225, N226, W227, E230, and K231, respectively in SEQ ID NO: 758).

In some aspects, the anti-FIXa antibody, or antigen binding portion thereof, binds to an epitope comprises chymotrypsinogen numbering amino acid residues H91, H92, N93, H101, D125, K126, E127, Y128, R165, Y177, N178, N179, S232, R233, Y234, V235, N236, W237, E240, and K241 in the sequence of the heavy chain of FIXa (corresponding to positions H76, H77, N78, H88, D112, K113, E114, Y115, R153, Y165, N166, N167, S222, R223, Y224, V225, N226, W227, E230, and K231, respectively in SEQ ID NO: 758).

In some aspects, the anti-FIXa antibody, or antigen binding portion thereof, binds to an epitope consisting of chymotrypsinogen numbering amino acid residues H91, H92, N93, H101, D125, K126, E127, Y128, R165, Y177, N178, N179, S232, R233, Y234, V235, N236, W237, E240, and K241 in the sequence of the heavy chain of FIXa (corresponding to positions H76, H77, N78, H88, D112, K113, E114, Y115, R153, Y165, N166, N167, S222, R223, Y224, V225, N226, W227, E230, and K231, respectively in SEQ ID NO: 758).

In some aspects, the anti-FIXa antibody, or antigen binding portion thereof, binds to an epitope comprising chymotrypsinogen numbering amino acid residues N93, R165, N178, and R233 in the sequence of the heavy chain of FIXa (corresponding to positions N78, R153, N166 and R223, respectively in SEQ ID NO: 758).

In some aspects, the anti-FIXa antibody, or antigen binding portion thereof, binds to an epitope which does not comprise at least one of chymotrypsinogen numbering amino acid residues N100, K132, Y137, R170, T172, F174, T175, H185, E202, and G205 in the sequence of the heavy chain of FIXa (corresponding to positions N87, K121, Y126, R158, T160, F162, T163, H174, E192 and G195, respectively in SEQ ID NO: 758). In some aspects, the anti-FIXa antibody, or antigen binding portion thereof, binds to an epitope which does not comprise chymotrypsinogen numbering amino acid residues N100, K132, Y137, R170, T172, F174, T175, H185, E202, and G205 in the sequence of the heavy chain of FIXa (corresponding to positions N87, K121, Y126, R158, T160, F162, T163, H174, E192 and G195, respectively in SEQ ID NO: 758).

In some aspects, the anti-FIXa antibody, or antigen binding portion thereof, binds to an epitope comprising at least one amino acid residue in the light chain of FIXa (SEQ ID NO:756). In some aspects, the epitope in the light chain of FIXa (SEQ ID NO:756) that the anti-FIXa antibody, or antigen binding portion thereof binds to is K100.

In some aspects, the anti-FIXa antibody, or antigen binding portion thereof, binds to an epitope comprising chymotrypsinogen numbering amino acid residues H91, H92, N93, H101, D125, K126, E127, Y128, R165, Y177, N178, N179, S232, R233, Y234, V235, N236, W237, E240, and K241 in the sequence of the heavy chain of FIXa (corresponding to positions H76, H77, N78, H88, D112, K113, E114, Y115, R153, Y165, N166, N167, S222, R223, Y224, V225, N226, W227, E230, and K231, respectively in SEQ ID NO: 758) and amino acid residue K100 of the sequence of the light chain of FIXa (SEQ ID NO:756). In some aspects, the anti-FIXa antibody, or antigen binding portion thereof, binds to an epitope consisting of chymotrypsinogen numbering amino acid residues H91, H92, N93, H101, D125, K126, E127, Y128, R165, Y177, N178, N179, S232, R233, Y234, V235, N236, W237, E240, and K241 in the sequence of the heavy chain of FIXa (corresponding to positions H76, H77, N78, H88, D112, K113, E114, Y115, R153, Y165, N166, N167, S222, R223, Y224, V225, N226, W227, E230, and K231, respectively in SEQ ID NO: 758) and amino acid residue K100 of the sequence of the light chain of FIXa (SEQ ID NO:756).

In some aspects, the anti-FIXa antibody, or antigen binding portion thereof, binds to an epitope that overlaps the binding site of FVIIIa to FIXa. In some aspects, the anti-FIXa antibody, or antigen binding portion thereof, cross-competes with FVIIIa for binding to FIXa. In some aspects, the anti-FXa antibody, or antigen binding portion thereof, blocks binding of FVIIIa to FIXa.

In some aspects, the anti-FIXa antibody, or antigen binding portion thereof, which specifically binds to FIXa, comprises VH CDR1, CDR2, and CDR3 sequences, wherein the VH CDR1 sequence comprises a VH CDR1 sequence selected from the group consisting of VH CDR1 sequences disclosed in TABLE 7 or a VH CDR1 sequence disclosed in TABLE 7 with one or two mutations.

In some aspects, the anti-FIXa antibody, or antigen binding portion thereof, which specifically binds to FIXa, comprises VH CDR1, CDR2, and CDR3 sequences, wherein the VH CDR2 sequence comprises a VH CDR2 sequence selected from the group consisting of VH CDR2 sequences disclosed in TABLE 7 or a VH CDR2 sequence disclosed in TABLE 7 with one or two mutations.

In some aspects, the anti-FIXa antibody, or antigen binding portion thereof, which specifically binds to FIXa, comprises VH CDR1, CDR2, and CDR3 sequences, wherein the VH CDR3 sequence comprises a VH CDR3 sequence selected from the group consisting of VH CDR3 sequence disclosed in TABLE 7 or a VH CDR3 sequence disclosed in TABLE 7 with one or two mutations.

In some aspects, the anti-FIXa antibody, or antigen binding portion thereof, which specifically binds to FIXa, comprises VL CDR1, CDR2, and CDR3 sequences, wherein the VL CDR1 sequence comprises a VL CDR1 sequence selected from the group consisting of VL CDR1 sequences disclosed in TABLE 7 or a VL CDR1 sequence disclosed in TABLE 7 with one or two mutations.

In some aspects, the anti-FIXa antibody, or antigen binding portion thereof, which specifically binds to FIXa, comprises VL CDR1, CDR2, and CDR3 sequences, wherein the VL CDR2 sequence comprises a VL CDR2 sequence selected from the group consisting of VL CDR2 sequences disclosed in TABLE 7 or a VL CDR2 sequence disclosed in TABLE 7 with one or two mutations.

In some aspects, the anti-FIXa antibody, or antigen binding portion thereof, which specifically binds to FIXa, comprises VL CDR1, CDR2, and CDR3 sequences, wherein the VL CDR3 sequence comprises a VL CDR3 sequence selected from the group consisting of VL CDR3 sequences disclosed in TABLE 7 or a VL CDR3 sequence disclosed in TABLE 7 with one or two mutations.

In some aspects, the isolated anti-FIXa antibody, or antigen binding portion thereof, comprises VH CDR1, CDR2, and CDR3 sequences, and VL CDR1, CDR2, and CDR3 sequences, wherein the VH CDR1, CDR2, and CDR3 sequences and the VL CDR1, CDR2, and CDR3 sequences comprise VH CDR1, CDR2, and CDR3 sequences and VL CDR1, CDR2, and CDR3 sequences disclosed in TABLE 7, respectively.

In some aspects, the isolated anti-FIXa antibody, or antigen binding portion thereof, comprises VH CDR1, CDR2 and CDR3, wherein
(i) the VH CDR1 comprises the amino acid sequence FTFX₁SX₂X₃MX₄ (SEQ ID NO: 2194), wherein X₁ is S, G or E, X₂ is Y or F, X₃ is S, E, G, or D, and X₄ is N, V, A, or T; and/or,
(ii) the VH CDR2 comprises the amino acid sequence X₅ISX₆X₇X₈X₉X₁₀IYYADSVKG (SEQ ID NO: 2195), wherein X₅ is S, A, Y, or G, X₆ is S or A, X₇ is S, A, or G, X₈ is S, G, or D, X₉ is S, T, or G, and X₁₀ is Y or T; and/or,
(iii) the VH CDR3 comprises the amino acid sequence ARDX₁₁GGYAGYYGMDV (SEQ ID NO: 2196), wherein X₁₁ is L or V.

In some aspects, the isolated anti-FIXa antibody, or antigen binding portion thereof, comprises VL CDR1, CDR2 and CDR3, wherein

(i) the VL CDR1 comprises the amino acid sequence QASQDIANYLN (SEQ ID NO:2116); and/or,

(ii) the VL CDR2 comprises the amino acid sequence DASNLET (SEQ ID NO:2142); and/or,

(iii) the VL CDR3 comprises the amino acid sequence QQYANFPYT (SEQ ID NO:2168).

In some aspects, the isolated anti-FIXa antibody, or antigen binding portion thereof, comprises VH CDR1, CDR2 and CDR3, wherein

(i) the VH CDR1 comprises the amino acid sequence FTFX₁SX₂X₃MX₄(SEQ ID NO: 2194), wherein X₁ is S, G or E, X₂ is Y or F, X₃ is S, E, G, or D, and X₄ is N, V, A, or T; and/or,

(ii) the VH CDR2 comprises the amino acid sequence X₅ISX₆X₇X₈X₉X₁₀IYYADSVKG (SEQ ID NO: 2195), wherein X₅ is S, A, Y, or G, X₆ is S or A, X₇ is S, A, or G, X₈ is S, G, or D, X₉ is S, T, or G, and X₁₀ is Y or T; and/or,

(iii) the VH CDR3 comprises the amino acid sequence ARDX₁₁GGYAGYYGMDV (SEQ ID NO: 2196), wherein X₁₁ is L or V; and,

further comprises VL CDR1, CDR2 and CDR3, wherein VL CDR1 comprises the amino acid sequence QASQDIANYLN (SEQ ID NO:2116); and/or, VL CDR2 comprises the amino acid sequence DASNLET (SEQ ID NO:2142); and/or, VL CDR3 comprises the amino acid sequence QQYANFPYT (SEQ ID NO:2168).

In some aspects the anti-FIXa antibody, or antigen binding portion thereof, comprises VH CDR1, CDR2, and CDR3 sequences comprising a VH CDR1 selected from SEQ ID NOs: 2038 to 2047, a VH CDR2 selected from SEQ ID NOs: 2064 to 2073, and a VH CDR3 selected from SEQ ID NOs: 2090 to 2099, and/or VL CDR1, CDR2, and CDR3 sequences comprising a VL CDR1 selected from SEQ ID NOs: 2116 to 2125, a VL CDR2 selected from SEQ ID NOs: 2142 to 2151, and a VL CDR3 selected from SEQ ID NOs: 2168 to 2177.

In some aspects, the isolated anti-FIXa antibody, or antigen binding portion thereof, comprises VH CDR1, CDR2 and CDR3, wherein
(i) the VH CDR1 comprises the amino acid sequence FTFGSYDMN (SEQ ID NO: 2048); and/or,
(ii) the VH CDR2 comprises the amino acid sequence SISX₁X₂X₃SYIX₄YAX₅SVKG (SEQ ID NO: 2197), wherein X₁ is S or D, X₂ is G or S, X₃ is E or A, X₄ is Y or A, and X₅ is E or D; and/or,
(iii) the VH CDR3 comprises the amino acid sequence X₆RDVX₇GYAGX₈YGMDV (SEQ ID NO: 2198), wherein X₆ is A or V, X₇ is G or S, and X₈ is Y or F.

In some aspects, the isolated anti-FIXa antibody, or antigen binding portion thereof, comprises VL CDR1, CDR2 and CDR3, wherein
(i) the VL CDR1 comprises the amino acid sequence X₁AX₂X₃X₄IX₅X₆YLN (SEQ ID NO: 2199), wherein X₁ is Q, G, or E, X₂ is S or N, X₃ is Q or E, X₄ is D or Y, X₅ is A or S, X₆ is N or D; and/or,
(ii) the VL CDR2 comprises the amino acid sequence DAX₇NLX₈X₉(SEQ ID NO: 2200), wherein X₇ is S or A, X₈ is E, H or Q, and X₉ is T or Y; and/or,
(iii) the VL CDR3 comprises the amino acid sequence X₁₀QYAX₁₁FPYT (SEQ ID NO: 2201), wherein X₁₀ is Q or S, and X₁₁ is N or R.

In some aspects, the isolated anti-FIXa antibody, or antigen binding portion thereof, comprises VH CDR1, CDR2 and CDR3, wherein
(i) the VH CDR1 comprises the amino acid sequence FTFGSYDMN (SEQ ID NO: 2048); and/or,
(ii) the VH CDR2 comprises the amino acid sequence SISX₁X₂X₃SYIX₄YAX₅SVKG (SEQ ID NO: 2197), wherein X₁ is S or D, X₂ is G or S, X₃ is E or A, X₄ is Y or A, and X₅ is E or D; and/or,
(iii) the VH CDR3 comprises the amino acid sequence X₆RDVX₇GYAGX₈YGMDV (SEQ ID NO: 2198), wherein X₆ is A or V, X₇ is G or S, and X₈ is Y or F; and
   further comprises VL CDR1, CDR2 and CDR3, wherein
(iv) the VL CDR1 comprises the amino acid sequence X₁AX₂X₃X₄IX₅X₆YLN (SEQ ID NO: 2199), wherein X₁ is Q, G, or E, X₂ is S or N, X₃ is Q or E, X₄ is D or Y, X₅ is A or S, X₆ is N or D; and/or,
(v) the VL CDR2 comprises the amino acid sequence DAX₇NLX₈X₉(SEQ ID NO: 2200), wherein X₇ is S or A, X₈ is E, H or Q, and X₉ is T or Y; and/or,
(vi) the VL CDR3 comprises the amino acid sequence X₁₀QYAX₁₁FPYT (SEQ ID NO: 2201), wherein X₁₀ is Q or S, and X₁₁ is N or R.

In some aspects, the anti-FIXa antibody, or antigen binding portion thereof, comprises VH CDR1, CDR2, and CDR3 sequences comprising a VH CDR1 selected from SEQ ID NOs: 2048 to 2052, a VH CDR2 selected from SEQ ID Nos: 2074 to 2078, and a VH CDR3 selected from SEQ ID NOs: 2100 to 2104, and/or VL CDR1, CDR2, and CDR3 sequences comprising a VL CDR1 selected from SEQ ID NOs: 2126 to 2130, a VL CDR2 selected from SEQ ID NOs: 2152 to 2156, and a VL CDR3 selected from SEQ ID NOs: 2178 to 2182.

In some aspects, the isolated anti-FIXa antibody, or antigen binding portion thereof, comprises VH CDR1, CDR2 and CDR3, wherein
(i) the VH CDR1 comprises the amino acid sequence YTFX₁X₂YX₃MH (SEQ ID NO: 2202), wherein X₁ is T or H, X₂ is S, G, or H, and X₃ is Y or P; and/or,
(ii) the VH CDR2 comprises the amino acid sequence X₄INPSX₅GX₆TX₇YAQKFQG (SEQ ID NO: 2203), wherein X₄ is I or S, X₅ is G or R, X₆ is S or R, and X₇ is S or E; and/or,
(iii) the VH CDR3 comprises the amino acid sequence ARDGPX₈X₉X₁₀DYYMDV (SEQ ID NO: 2204), wherein X₈ is R or Q, X₉ is V, D, L or E, and X₁₀ is S or V.

In some aspects, the isolated anti-FIXa antibody, or antigen binding portion thereof, comprises VL CDR1, CDR2 and CDR3, wherein the VL CDR1 comprises the amino acid sequence RASQSVSSYLA (SEQ ID NO:2116); and/or, the VL CDR2 comprises the amino acid sequence DASNRAT (SEQ ID NO:2116); and/or, (iii) the VL CDR3 comprises the amino acid sequence QQRDNWPFT (SEQ ID NO:2116).

In some aspects, the isolated anti-FIXa antibody, or antigen binding portion thereof, comprises VH CDR1, CDR2 and CDR3, wherein
(i) the VH CDR1 comprises the amino acid sequence YTFX₁X₂YX₃MH (SEQ ID NO: 2202), wherein X₁ is T or H, X₂ is S, G, or H, and X₃ is Y or P; and/or,
(ii) the VH CDR2 comprises the amino acid sequence X₄INPSX₅GX₆TX₇YAQKFQG (SEQ ID NO: 2203), wherein X₄ is I or S, X₅ is G or R, X₆ is S or R, and X₇ is S or E; and/or,
(iii) the VH CDR3 comprises the amino acid sequence ARDGPX₈X₉X₁₀DYYMDV (SEQ ID NO: 2204), wherein X₈ is R or Q, X₉ is V, D, L or E, and X₁₀ is S or V; and
   further comprises VL CDR1, CDR2 and CDR3, wherein the VL CDR1 comprises the amino acid sequence RASQSVSSYLA (SEQ ID NO:2116); and/or, the VL CDR2 comprises the amino acid sequence DASNRAT (SEQ ID NO:2116); and/or, (iii) the VL CDR3 comprises the amino acid sequence QQRDNWPFT (SEQ ID NO:2116).

In some aspects, the anti-FIXa antibody, or antigen binding portion thereof, comprises VH CDR1, CDR2, and CDR3 sequences comprising a VH CDR1 selected from SEQ ID NOs: 2053 to 2057, a VH CDR2 selected from SEQ ID NOs: 2079 to 2083, and a VH CDR3 selected from SEQ ID NOs: 2105 to 2109, and/or VL CDR1, CDR2, and CDR3 sequences comprising a VL CDR1 selected from SEQ ID NOs: 2131 to 2135, a VL CDR2 selected from SEQ ID NOs: 2157 to 2161, and a VL CDR3 selected from SEQ ID NOs: 2183 to 2187.

In some aspects, the isolated anti-FIXa antibody, or antigen binding portion thereof, comprises VH CDR1, CDR2 and CDR3, wherein
(i) the VH CDR1 comprises the amino acid sequence GSIX₁SX₂X₃YX₄WX₅(SEQ ID NO: 2205), wherein X₁ is S or A, X₂ is S, T, G, or V, X₃ is S or A, X₄ is Y or A, and X₅ is G, V, N, or S; and/or
(ii) the VH CDR2 comprises the amino acid sequence X₆IX₇X₈X₉GX₁₀TX₁₁YNPSLKS (SEQ ID NO: 2206), wherein X₆ is S or Y, X₇ is S, Y, R, T or Q, X₈ is Y, G, P or A, X₉ is S or Q, X₁₀ is S or K, and X₁₁ is Y or Q; and/or,
(iii) the VH CDR3 comprises the amino acid sequence ARDKYQDYSX₁₂DI, wherein X₁₂ is F or V (SEQ ID NO: 2207).

In some aspects, the isolated anti-FIXa antibody, or antigen binding portion thereof, comprises VL CDR1, CDR2 and CDR3, wherein the VL CDR1 comprises the amino acid sequence RASQGIDSWLA (SEQ ID NO:2136); and/or, the VL CDR2 comprises the amino acid sequence AASSLQS (SEQ ID NO:2162); and/or, the VL CDR3 comprises the amino acid sequence QQANFLPFT (SEQ ID NO:2188).

In some aspects, the isolated anti-FIXa antibody, or antigen binding portion thereof, comprises VH CDR1, CDR2 and CDR3, wherein
(i) the VH CDR1 comprises the amino acid sequence GSIX₁SX₂X₃YX₄WX₅ (SEQ ID NO: 2205), wherein X₁ is S or A, X₂ is S, T, G, or V, X₃ is S or A, X₄ is Y or A, and X₅ is G, V, N, or S; and/or,
(ii) the VH CDR2 comprises the amino acid sequence X₆IX₇X₈X₉GX₁₀TX₁₁YNPSLKS (SEQ ID NO: 2206), wherein X₆ is S or Y, X₇ is S, Y, R, T or Q, X₈ is Y, G, P or A, X₉ is S or Q, X₁₀ is S or K, and X₁₁ is Y or Q; and/or,
(iii) the VH CDR3 comprises the amino acid sequence ARDKYQDYSX₁₂DI (SEQ ID NO: 2207), wherein X₁₂ is F or V; and
   further comprises VL CDR1, CDR2 and CDR3, wherein the VL CDR1 comprises the amino acid sequence RASQGIDSWLA (SEQ ID NO:2136); and/or, the VL CDR2 comprises the amino acid sequence AASSLQS (SEQ ID NO:2162); and/or, and the VL CDR3 comprises the amino acid sequence QQANFLPFT (SEQ ID NO:2188).

In some aspects, the anti-FIXa antibody, or antigen binding portion thereof, comprises VH CDR1, CDR2, and CDR3 sequences comprising a VH CDR1 selected from SEQ ID NOs: 2058 to 2063, a VH CDR2 selected from SEQ ID NOs: 2084 to 2089, and a VH CDR3 selected from SEQ ID NOs: 2110 to 2115, and/or VL CDR1, CDR2, and CDR3 sequences comprising a VL CDR1 selected from SEQ ID NOs: 2136 to 2141, a VL CDR2 selected from SEQ ID NOs: 2162 to 2167, and a VL CDR3 selected from SEQ ID NOs: 2188 to 2193.

In some aspects, the anti-FIXa antibody, or antigen binding portion thereof, comprises a VH and a VL, wherein the VH comprises an amino acid sequence which is at least about 80%, at least about 85%, at least about 90%, at least about 95%, at least about 96%, at least about 97%, at least about 98%, at least about 99%, or about 100% identical to the amino acid sequence selected from the group consisting of SEQ ID NOs: 1935, 1939, 1943, 1947, 1951, 1955, 1959, 1963, 1967, 1971, 1975, 1979, 1983, 1987, 1991, 1995, 1999, 2003, 2007, 2011, 2015, 2019, 2023, 2027, 2031, and 2035.

In some aspects, the anti-FIXa antibody, or antigen binding portion thereof, comprises a VH and a VL, wherein the VL comprises an amino acid sequence which is at least about 80%, at least about 85%, at least about 90%, at least about 95%, at least about 96%, at least about 97%, at least about 98%, at least about 99%, or about 100% identical to an amino acid sequence selected from the group consisting of SEQ ID NOs: 1937, 1941, 1945, 1949, 1953, 1957, 1961, 1965, 1969, 1973, 1977, 1981, 1985, 1989, 1993, 1997, 2001, 2005, 2009, 2013, 2017, 2021, 2025, 2029, 2033, and 2037.

In some aspects, the anti-FIXa antibody, or antigen binding portion thereof, comprises a VH and a VL, wherein

(i) the VH comprises an amino acid sequence which is at least about 80%, at least about 85%, at least about 90%, at least about 95%, at least about 96%, at least about 97%, at least about 98%, at least about 99%, or about 100% identical to the amino acid sequence selected from the group consisting of SEQ ID NOs: 1935, 1939, 1943, 1947, 1951, 1955, 1959, 1963, 1967, 1971, 1975, 1979, 1983, 1987, 1991, 1995, 1999, 2003, 2007, 2011, 2015, 2019, 2023, 2027, 2031, and 2035; and/or,
(ii) the VL comprises an amino acid sequence which is at least about 80%, at least about 85%, at least about 90%, at least about 95%, at least about 96%, at least about 97%, at least about 98%, at least about 99%, or about 100% identical to an amino acid sequence selected from the group consisting of SEQ ID NOs: 1937, 1941, 1945, 1949, 1953, 1957, 1961, 1965, 1969, 1973, 1977, 1981, 1985, 1989, 1993, 1997, 2001, 2005, 2009, 2013, 2017, 2021, 2025, 2029, 2033, and 2037.

In some aspects, the anti-FIXa antibody, or antigen binding portion thereof, comprises a VH and a VL, wherein the VH comprises an amino acid sequence at least 70%, at least 80%, at least 90%, at least 95%, at least 96%, at least 97%, at least 98%, at least 99%, or 100% identical to SEQ ID NO: 89 and VL comprises an amino acid sequence at least 70%, at least 80%, at least 90%, at least 95%, at least 96%, at least 97%, at least 98%, at least 99%, or 100% identical to SEQ ID NO: 221 (VH and VL of BIIB-9-1336, respectively).

In some aspects, the anti-FIXa antibody, or antigen binding portion thereof, comprises a VH and a VL, wherein
(a1) the VH comprises an amino acid sequence at least 70%, at least 80%, at least 90%, at least 95%, at least 96%, at least 97%, at least 98%, at least 99%, or 100% identical to SEQ ID NOs: 1935 and the VL comprises an amino acid sequence at least 70%, at least 80%, at least 90%, at least 95%, at least 96%, at least 97%, at least 98%, at least 99%, or 100% identical to SEQ ID NO: 1937;
(a2) the VH comprises an amino acid sequence at least 70%, at least 80%, at least 90%, at least 95%, at least 96%, at least 97%, at least 98%, at least 99%, or 100% identical to SEQ ID NOs: 1939 and the VL comprises an amino acid sequence at least 70%, at least 80%, at least 90%, at least 95%, at least 96%, at least 97%, at least 98%, at least 99%, or 100% identical to SEQ ID NO: 1941;
(a3) the VH comprises an amino acid sequence at least 70%, at least 80%, at least 90%, at least 95%, at least 96%, at least 97%, at least 98%, at least 99%, or 100% identical to SEQ ID NOs: 1943 and the VL comprises an amino acid sequence at least 70%, at least 80%, at least 90%, at least 95%, at least 96%, at least 97%, at least 98%, at least 99%, or 100% identical to SEQ ID NO: 1945;
(a4) the VH comprises an amino acid sequence at least 70%, at least 80%, at least 90%, at least 95%, at least 96%, at least 97%, at least 98%, at least 99%, or 100% identical to SEQ ID NOs: 1947 and the VL comprises an amino acid sequence at least 70%, at least 80%, at least 90%, at least 95%, at least 96%, at least 97%, at least 98%, at least 99%, or 100% identical to SEQ ID NO: 1949;
(a5) the VH comprises an amino acid sequence at least 70%, at least 80%, at least 90%, at least 95%, at least 96%, at least 97%, at least 98%, at least 99%, or 100% identical to SEQ ID NOs: 1951 and the VL comprises an amino acid sequence at least 70%, at least 80%, at least 90%, at least 95%, at least 96%, at least 97%, at least 98%, at least 99%, or 100% identical to SEQ ID NO: 1953;
(a6) the VH comprises an amino acid sequence at least 70%, at least 80%, at least 90%, at least 95%, at least 96%, at least 97%, at least 98%, at least 99%, or 100% identical to SEQ ID NOs: 1955 and the VL comprises an amino acid sequence at least 70%, at least 80%, at least 90%, at least 95%, at least 96%, at least 97%, at least 98%, at least 99%, or 100% identical to SEQ ID NO: 1957;
(a7) the VH comprises an amino acid sequence at least 70%, at least 80%, at least 90%, at least 95%, at least 96%, at least 97%, at least 98%, at least 99%, or 100% identical to SEQ ID NOs: 1959 and the VL comprises an amino acid sequence at least 70%, at least 80%, at least 90%, at least 95%, at least 96%, at least 97%, at least 98%, at least 99%, or 100% identical to SEQ ID NO: 1961;
(a8) the VH comprises an amino acid sequence at least 70%, at least 80%, at least 90%, at least 95%, at least 96%, at least 97%, at least 98%, at least 99%, or 100% identical to SEQ ID NOs: 1963 and the VL comprises an amino acid sequence at least 70%, at least 80%, at least 90%, at least 95%, at least 96%, at least 97%, at least 98%, at least 99%, or 100% identical to SEQ ID NO: 1965;
(a9) the VH comprises an amino acid sequence at least 70%, at least 80%, at least 90%, at least 95%, at least 96%, at least 97%, at least 98%, at least 99%, or 100% identical to SEQ ID NOs: 1967 and the VL comprises an amino acid sequence at least 70%, at least 80%, at least 90%, at least 95%, at least 96%, at least 97%, at least 98%, at least 99%, or 100% identical to SEQ ID NO: 1969;
(a10) the VH comprises an amino acid sequence at least 70%, at least 80%, at least 90%, at least 95%, at least 96%, at least 97%, at least 98%, at least 99%, or 100% identical to SEQ ID NOs: 1971 and the VL comprises an amino acid sequence at least 70%, at least 80%, at least 90%, at least 95%, at least 96%, at least 97%, at least 98%, at least 99%, or 100% identical to SEQ ID NO: 1973;
(a11) the VH comprises an amino acid sequence at least 70%, at least 80%, at least 90%, at least 95%, at least 96%, at least 97%, at least 98%, at least 99%, or 100% identical to SEQ ID NOs: 1975 and the VL comprises an amino acid sequence at least 70%, at least 80%, at least 90%, at least 95%, at least 96%, at least 97%, at least 98%, at least 99%, or 100% identical to SEQ ID NO: 1977;
(a12) the VH comprises an amino acid sequence at least 70%, at least 80%, at least 90%, at least 95%, at least 96%, at least 97%, at least 98%, at least 99%, or 100% identical to SEQ ID NOs: 1979 and the VL comprises an amino acid sequence at least 70%, at least 80%, at least 90%, at least 95%, at least 96%, at least 97%, at least 98%, at least 99%, or 100% identical to SEQ ID NO: 1981;
(a13) the VH comprises an amino acid sequence at least 70%, at least 80%, at least 90%, at least 95%, at least 96%, at least 97%, at least 98%, at least 99%, or 100% identical to SEQ ID NOs: 1983 and the VL comprises an amino acid sequence at least 70%, at least 80%, at least 90%, at least 95%, at least 96%, at least 97%, at least 98%, at least 99%, or 100% identical to SEQ ID NO: 1985;
(a14) the VH comprises an amino acid sequence at least 70%, at least 80%, at least 90%, at least 95%, at least 96%, at least 97%, at least 98%, at least 99%, or 100% identical to SEQ ID NOs: 1987 and the VL comprises an amino acid sequence at least 70%, at least 80%, at least 90%, at least 95%, at least 96%, at least 97%, at least 98%, at least 99%, or 100% identical to SEQ ID NO: 1989;
(a15) the VH comprises an amino acid sequence at least 70%, at least 80%, at least 90%, at least 95%, at least 96%, at least 97%, at least 98%, at least 99%, or 100% identical to SEQ ID NOs: 1991 and the VL comprises an amino acid sequence at least 70%, at least 80%, at least 90%, at least 95%, at least 96%, at least 97%, at least 98%, at least 99%, or 100% identical to SEQ ID NO: 1993;
(a16) the VH comprises an amino acid sequence at least 70%, at least 80%, at least 90%, at least 95%, at least 96%, at least 97%, at least 98%, at least 99%, or 100% identical to SEQ ID NOs: 1995 and the VL comprises an amino acid sequence at least 70%, at least 80%, at least 90%, at least 95%, at least 96%, at least 97%, at least 98%, at least 99%, or 100% identical to SEQ ID NO: 1997;
(a17) the VH comprises an amino acid sequence at least 70%, at least 80%, at least 90%, at least 95%, at least 96%, at least 97%, at least 98%, at least 99%, or 100% identical to SEQ ID NOs: 1999 and the VL comprises an amino acid sequence at least 70%, at least 80%, at least 90%, at least 95%, at least 96%, at least 97%, at least 98%, at least 99%, or 100% identical to SEQ ID NO: 2001;
(a18) the VH comprises an amino acid sequence at least 70%, at least 80%, at least 90%, at least 95%, at least 96%, at least 97%, at least 98%, at least 99%, or 100% identical to SEQ ID NOs: 2003 and the VL comprises an amino acid sequence at least 70%, at least 80%, at least 90%, at least 95%, at least 96%, at least 97%, at least 98%, at least 99%, or 100% identical to SEQ ID NO: 2005;
(a19) the VH comprises an amino acid sequence at least 70%, at least 80%, at least 90%, at least 95%, at least 96%, at least 97%, at least 98%, at least 99%, or 100% identical to SEQ ID NOs: 2007 and the VL comprises an amino acid sequence at least 70%, at least 80%, at least 90%, at least 95%, at least 96%, at least 97%, at least 98%, at least 99%, or 100% identical to SEQ ID NO: 2009;
(a20) the VH comprises an amino acid sequence at least 70%, at least 80%, at least 90%, at least 95%, at least 96%, at least 97%, at least 98%, at least 99%, or 100% identical to SEQ ID NOs: 2011 and the VL comprises an amino acid sequence at least 70%, at least 80%, at least 90%, at least 95%, at least 96%, at least 97%, at least 98%, at least 99%, or 100% identical to SEQ ID NO: 2013;
(a21) the VH comprises an amino acid sequence at least 70%, at least 80%, at least 90%, at least 95%, at least 96%, at least 97%, at least 98%, at least 99%, or 100% identical to SEQ ID NOs: 2015 and the VL comprises an amino acid sequence at least 70%, at least 80%, at least 90%, at least 95%, at least 96%, at least 97%, at least 98%, at least 99%, or 100% identical to SEQ ID NO: 2017;
(a22) the VH comprises an amino acid sequence at least 70%, at least 80%, at least 90%, at least 95%, at least 96%, at least 97%, at least 98%, at least 99%, or 100% identical to SEQ ID NOs: 2019 and the VL comprises an amino acid sequence at least 70%, at least 80%, at least 90%, at least 95%, at least 96%, at least 97%, at least 98%, at least 99%, or 100% identical to SEQ ID NO: 2021;
(a23) the VH comprises an amino acid sequence at least 70%, at least 80%, at least 90%, at least 95%, at least 96%, at least 97%, at least 98%, at least 99%, or 100% identical to SEQ ID NOs: 2023 and the VL comprises an amino acid sequence at least 70%, at least 80%, at least 90%, at least 95%, at least 96%, at least 97%, at least 98%, at least 99%, or 100% identical to SEQ ID NO: 2025;
(a24) the VH comprises an amino acid sequence at least 70%, at least 80%, at least 90%, at least 95%, at least 96%, at least 97%, at least 98%, at least 99%, or 100% identical to SEQ ID NOs: 2027 and the VL comprises an amino acid sequence at least 70%, at least 80%, at least 90%, at least 95%, at least 96%, at least 97%, at least 98%, at least 99%, or 100% identical to SEQ ID NO: 2029;
(a25) the VH comprises an amino acid sequence at least 70%, at least 80%, at least 90%, at least 95%, at least 96%, at least 97%, at least 98%, at least 99%, or 100% identical to SEQ ID NOs: 2031 and the VL comprises an amino acid sequence at least 70%, at least 80%, at least 90%, at least 95%, at least 96%, at least 97%, at least 98%, at least 99%, or 100% identical to SEQ ID NO: 2033; or,
(a26) the VH comprises an amino acid sequence at least 70%, at least 80%, at least 90%, at least 95%, at least 96%, at least 97%, at least 98%, at least 99%, or 100% identical to SEQ ID NOs: 2035 and the VL comprises an amino acid sequence at least 70%, at least 80%, at least 90%, at least 95%, at least 96%, at least 97%, at least 98%, at least 99%, or 100% identical to SEQ ID NO: 2037.

In some aspects, the anti-FIXa antibody, or antigen binding portion thereof, comprises a VH and a VL, wherein (a1) the VH and the VL comprise SEQ ID NOs: 1935 and 1937, respectively (BIIB-9-3595);
(a2) the VH and the VL comprise SEQ ID NOs: 1939 and 1941, respectively (BIIB-9-3601);
(a3) the VH and the VL comprise SEQ ID NOs: 1943 and 1945, respectively (BIIB-9-3604);
(a4) the VH and the VL comprise SEQ ID NOs: 1947 and 1949, respectively (BIIB-9-3617);
(a5) the VH and the VL comprise SEQ ID NOs: 1951 and 1953, respectively (BIIB-9-3618);
(a6) the VH and the VL comprise SEQ ID NOs: 1955 and 1957, respectively (BIIB-9-3621);
(a7) the VH and the VL comprise SEQ ID NOs: 1959 and 1961, respectively (BIIB-9-3647);
(a8) the VH and the VL comprise SEQ ID NOs: 1963 and 1965, respectively (BIIB-9-3649);
(a9) the VH and the VL comprise SEQ ID NOs: 1967 and 1969, respectively (BIIB-9-3650);
(a10) the VH and the VL comprise SEQ ID NOs: 1971 and 1973, respectively (BIIB-9-3654);
(a11) the VH and the VL comprise SEQ ID NOs: 1975 and 1977, respectively (BIIB-9-3753);
(a12) the VH and the VL comprise SEQ ID NOs: 1979 and 1981, respectively (BIIB-9-3754);
(a13) the VH and the VL comprise SEQ ID NOs: 1983 and 1985, respectively (BIIB-9-3756);
(a14) the VH and the VL comprise SEQ ID NOs: 1987 and 1989, respectively (BIIB-9-3764);
(a15) the VH and the VL comprise SEQ ID NOs: 1991 and 1993, respectively (BIIB-9-3766);
(a16) the VH and the VL comprise SEQ ID NOs: 1995 and 1997, respectively (BIIB-9-3707);
(a17) the VH and the VL comprise SEQ ID NOs: 1999 and 2001, respectively (BIIB-9-3709);
(a18) the VH and the VL comprise SEQ ID NOs: 2003 and 2005, respectively (BIIB-9-3720);
(a19) the VH and the VL comprise SEQ ID NOs: 2007 and 2009, respectively (BIIB-9-3727);
(a20) the VH and the VL comprise SEQ ID NOs: 2011 and 2013, respectively (BIIB-9-3745);
(a21) the VH and the VL comprise SEQ ID NOs: 2015 and 2017, respectively (BIIB-9-3780);
(a22) the VH and the VL comprise SEQ ID NOs: 2019 and 2021, respectively (BIIB-9-3675);
(a23) the VH and the VL comprise SEQ ID NOs: 2023 and 2025, respectively (BIIB-9-3681);
(a24) the VH and the VL comprise SEQ ID NOs: 2027 and 2029, respectively (BIIB-9-3684);
(a25) the VH and the VL comprise SEQ ID NOs: 2031 and 2033, respectively (BIIB-9-3698); or,
(a26) the VH and the VL comprise SEQ ID NOs: 2035 and 2037, respectively (BIIB-9-3704).

In some aspects, the binding of an anti-FIXa antibody disclosed herein (e.g., BIIB-9-484) to FIXa is calcium dependent. In other aspects, the binding of an anti-FIXa antibody disclosed herein to FIXa is calcium independent. In yet other aspects, the binding of the anti-FIXa antibody (e.g., BIIB-9-1336) to FIXa is partially calcium dependent.

In some aspects, an anti-FIXa antibody disclosed herein, e.g., BIIB-9-1336, can increase the amidolytic activity of FIXa. In some aspects, an anti-FIXa antibody disclosed herein (e.g., BIIB-9-1336, or a bispecific antibody comprising BIIB-9-1336) can increase the rate of substrate cleavage (e.g., ADG299) by FIXa. In some aspects, the binding of an anti-FIXa antibody disclosed herein, e.g., BIIB-9-1336, to FIXa can increase FIXa's amidolytic activity by at least 2-fold, at least 3-fold, or at least 4-fold. In some aspects, the binding of an anti-FIXa antibody disclosed herein, e.g., BIIB-9-1336, to FIXa can increase FIXa's amidolytic activity by at least 10%, at least 20%, at least 30%, at least 40%, at least 50%, at least 60%, at least 70%, at least 80%, at least 90%, at least 100%, at least 120%, at least 140%, at least 160%, at least 180%, at least 200%, at least 220%, at least 240%, at least 260%, at least 280%, at least 300%, at least 320%, at least 340%, at least 360%, at least 380%, at least 400%, at least 420%, at least 440%, at least 460%, at least 480%, or at least 500%.

The present disclosure provides a method to increase the amidolytic activity of FIXa comprising administering an anti-FIXa antibody disclosed here, e.g., BIIB-9-1336 or a bispecific antibody comprising BIIB-9-1336, to a subject in need thereof.

In some aspects, an anti-FIXa antibody disclosed herein can increase the rate of FIXa inhibition by anti-thrombin III (ATIII). In some aspects, an anti-FIXa antibody disclosed herein (e.g., BIIB-9-1336, or a bispecific antibody comprising BIIB-9-1336) can increase the rate of FIXa inhibition by ATIII. In some aspects, the rate of FIXa inhibition by ATIII is increased at least 2-fold, at least 3-fold, or at least 4-fold. In other aspects, binding of an anti-FIXa antibody disclosed herein, e.g., BIIB-9-1336, to FIXa can increase the rate of FIXa inhibition by ATIII by at least 10%, at least 20%, at least 30%, at least 40%, at least 50%, at least 60%, at least 70%, at least 80%, at least 90%, at least 100%, at least 120%, at least 140%, at least 160%, at least 180%, at least 200%, at least 220%, at least 240%, at least 260%, at least 280%, at least 300%, at least 320%, at least 340%, at least 360%, at least 380%, or at least 400%.

The present disclosure provides a method to increase rate of FIXa inhibition by ATIII comprising administering anti-FIXa antibody disclosed here, e.g., BIIB-9-1336 or a bispecific antibody comprising BIIB-9-1336, to a subject in need thereof.

### (b) Anti-FIXz Binding Molecules

The present disclosure therefore provides an antibody (e.g., an isolated antibody), or an antigen binding portion thereof, that specifically binds to FIX zymogen (FIXz), wherein the antibody or antigen binding portion thereof preferentially binds to FIXz in the presence of FIXa and FIXz (“anti-FIXz antibody or antigen binding portion thereof”).

In some aspects, the anti-FIXz antibody, or antigen binding portion thereof, binds to FIXz with a binding affinity higher than a binding affinity of the anti-FIXz antibody or antigen binding portion thereof to FIXa (e.g., free FIXa or FIXa-SM).

The present disclosure also provides an isolated anti-FIXz antibody, or antigen binding portion thereof, which binds to FIXz with a binding affinity higher than a binding affinity of the anti-FIXz antibody or antigen binding portion thereof to FIXa. In some aspects, the anti-FIXz antibody, or antigen binding portion thereof, binds to FIXz with a K_{D} of about 100 nM or less, about 95 nM or less, about 90 nM or less, about 85 nM or less, about 80 nM or less, about 75 nM or less, about 70 nM or less, about 65 nM or less, about 60 nM or less, about 55 nM or less, about 50 nM or less, about 45 nM or less, about 40 nM or less, about 35 nM or less, about 30 nM or less, about 25 nM or less, about 20 nM or less, about 15 nM or less, about 10 nM or less, about 5 nM or less, or about 1 nM or less as determined by a Bio-Layer Interferometry (BLI) assay. In other embodiments, the anti-FIXz antibody, or antigen binding portion thereof, binds to FIXz with a K_{D} of about 10 nM or less, about 9 nM or less, about 8 nM or less, about 7 nM or less, about 6 nM or less, about 5 nM or less, about 4 nM or less, about 3 nM or less, about 2 nM or less, about 1 nM or less, about 0.5 nM or less, about 0.2 nM or less, about 0.1 nM or less, or about 0.05 nM or less.

In some aspects, the anti-FIXz antibody, or antigen binding portion thereof, cross-competes with a reference antibody selected from the group consisting of the antibodies in FIG. 3D. In some aspects, the anti-FIXa antibody, or antigen binding portion thereof, binds to the same epitope as a reference antibody selected from the group consisting of the antibodies in FIG. 3D. In some aspects, the reference antibody is BIIB-9-578.

In further aspects, the anti-FIXz antibody or antigen binding portion thereof can be also referred to as Class IV: anti-FIXz antibodies or antigen binding portion thereof that preferentially binds to FIXz over free FIXa or FIXa-SM (FIG. 3D antibodies).

In some aspects, the anti-FIXz antibody, or antigen binding portion thereof, comprises a VH CDR1, a VH CDR2, and a VH CDR3, wherein the VH CDR3 comprises
(i) a VH CDR3 sequence identical to a VH CDR3 sequence selected from the group consisting of VH CDR3 sequences in FIG. 3D, or
(ii) a VH CDR3 sequence identical to a VH CDR3 sequence selected from the group consisting of VH CDR3 sequences in FIG. 3D except for 1, 2, or 3 amino acid substitutions.

In some aspects, the amino acid substitutions are conservative amino acid substitutions. In other aspects, the amino acid substitutions are back mutation.

In some aspects, the anti-FIXz antibody, or antigen binding portion thereof, comprises a VH CDR1, a VH CDR2, and a VH CDR3, wherein the VH CDR3 sequence comprises an amino acid sequence selected from ARDKYQDYSFDI (SEQ ID NO: 1355; BIIB-9-578). In some aspects, the VH CDR3 sequences disclosed herein can comprise 1, 2, or 3 amino acid substitutions.

In some aspects, the anti-FIXz antibody, or antigen binding portion thereof, comprises a VH CDR1, a VH CDR2, and VH CDR3, wherein the VH CDR3 comprises SEQ ID NO: 1355 (BIIB-9-578) and the VH CDR1 sequence comprises
(i) a VH CDR1 sequence identical to a sequence selected from the group consisting of the VH CDR1 sequences disclosed in FIG. 3D, or
(ii) a VH CDR1 sequence identical to a sequence selected from the group consisting of the VH CDR1 sequences disclosed in FIG. 3D except for 1, 2, or 3 amino acid substitutions.

In some aspects, the anti-FIXz antibody, or antigen binding portion thereof, comprises a VH CDR1, a VH CDR2, and VH CDR3, wherein the VH CDR3 comprises SEQ ID NO: 1355 (BIIB-9-578) and the VH CDR2 sequence comprises
(i) a VH CDR2 sequence identical to a sequence selected from the group consisting of the VH CDR2 sequences disclosed in FIG. 3D, or
(ii) a VH CDR2 sequence identical to a sequence selected from the group consisting of the VH CDR2 sequences disclosed in FIG. 3D except for 1, 2, or 3 amino acid substitutions.

In some aspects, the anti-FIXz antibody, or antigen binding portion thereof, further comprises a VL CDR1, a VL CDR2, and a VL CDR3, wherein the VL CDR1 sequence comprises
(i) a VL CDR1 sequence identical to a sequence selected from the group consisting of the VL CDR1 sequences disclosed in FIG. 3D, or
(ii) a VL CDR1 sequence identical to a sequence selected from the group consisting of the VL CDR1 sequences disclosed in FIG. 3D except for 1, 2, or 3 amino acid substitutions.

In some aspects, the anti-FIXz antibody, or antigen binding portion thereof, further comprises a VL CDR1, a VL CDR2, and a VL CDR3, wherein the VL CDR2 sequence comprises
(i) a VL CDR2 sequence identical to a sequence selected from the group consisting of the VL CDR2 sequences in FIG. 3D, or
(ii) a VL CDR2 sequence identical to a sequence selected from the group consisting of the VL CDR2 sequences in FIG. 3D except for 1, 2, or 3 amino acid substitutions.

In some aspects, the anti-FIXa antibody, or antigen binding portion thereof, further comprises a VL CDR1, a VL CDR2, and a VL CDR3, wherein the VL CDR3 sequence comprises
(i) a VL CDR3 sequence identical to a sequence selected from the group consisting of the VL CDR3 sequences disclosed in FIG. 3D, or
(ii) a VL CDR3 sequence identical to a sequence selected from the group consisting of the VL CDR3 sequences disclosed in FIG. 3D except for 1, 2, or 3 amino acid substitutions.

The present disclosure also provides an isolated antibody, or antigen binding portion thereof, which preferentially binds to FIXz, comprising a VH CDR1, a VH CDR2, and a VH CDR3, and a VL CDR1, a VL CDR2, and a VL CDR3, wherein the VH CDR1, VH CDR2, and VH CDR3 and the VL CDR1, VL CDR2, and VL CDR3 comprise the VH CDR1, VH CDR2, and VH CDR3 and the VL CDR1, VL CDR2, and VL CDR3 of an anti-FIXz antibody selected from the group consisting of the antibodies in FIG. 3D: BIIB-9-397, BIIB-9-578, BIIB-9-631, and BIIB-9-612.

In some aspects, the anti-FIXz antibody, or antigen binding portion thereof, comprises VH CDR1, VH CDR2, and VH CDR3 sequences comprising SEQ ID NOs: 1346-1349, SEQ ID NOs: 1350-1353, and SEQ ID NOs: 1354-1357, respectively (VH CDRs for Class IV antibodies), and/or VL CDR1, VL CDR2, and VL CDR3 sequences comprising SEQ ID NOs: 1358-1361, SEQ ID NOs: 1362-1365, and SEQ ID NO: 1366-1369, respectively (VL CDRs for Class IV antibodies).

In some aspects, the anti-FIXz antibody, or antigen binding portion thereof, comprises VH CDR1, VH CDR2, and VH CDR3 sequences comprising SEQ ID NO: 1347, SEQ ID NO: 1351, and SEQ ID NO: 1355, respectively (VH CDRs for BIIB-9-578 antibodies), and/or VL CDR1, VL CDR2, and VL CDR3 sequences comprising SEQ ID NO: 1359, SEQ ID NO: 1363, and SEQ ID NO: 1367, respectively (VL CDRs for BIIB-9-578 antibodies).

In some aspects, the anti-FIXz antibody, or antigen binding portion thereof, comprises a VH, wherein the VH comprises an amino acid sequence which is at least about 70%, at least about 75%, at least about 80%, at least about 85%, at least about 90%, at least about 95%, at least about 96%, at least about 97%, at least about 98%, at least about 99%, or about 100% identical to an amino acid sequence selected from the group consisting of SEQ ID NOs: 183, 185, 187, and 189.

In some aspects, the anti-FIXz antibody, or antigen binding portion thereof, comprises a VL, wherein the VL comprises an amino acid sequence which is at least about 70%, at least about 75%, at least about 80%, at least about 85%, at least about 90%, at least about 95%, at least about 96%, at least about 97%, at least about 98%, at least about 99%, or about 100% identical an the amino acid sequence selected from the group consisting of SEQ ID NOs: 369, 371, 373, and 375.

In some aspects, the anti-FIXz antibody, or antigen binding portion thereof, comprises a VH and a VL, wherein
(i) the VH comprises an amino acid sequence which is at least about 70%, at least about 75%, at least about 80%, at least about 85%, at least about 90%, at least about 95%, at least about 96%, at least about 97%, at least about 98%, at least about 99%, or about 100% identical to an amino acid sequence selected from the group consisting of SEQ ID NOs: 183, 185, 187, and 189; and,
(ii) the VL comprises an amino acid sequence which is at least about 70%, at least about 75%, at least about 80%, at least about 85%, at least about 90%, at least about 95%, at least about 96%, at least about 97%, at least about 98%, at least about 99%, or about 100% identical to an amino acid sequence selected from the group consisting of SEQ ID NOs: 369, 371, 373, and 375.

In certain embodiments, an anti-FIXz antibody comprises a heavy chain variable region from a particular germline heavy chain immunoglobulin gene and/or a light chain variable region from a particular germline light chain immunoglobulin gene. In some embodiments, the VH sequence of the anti-FIXz antibody can be derived from any one of V, D, or J germline sequences and/or the VL sequence of the anti-FIXz antibody can be derived from any one of kappa or lambda germline sequence.

As demonstrated herein, human antibodies specific for FIXz have been prepared that comprise a heavy chain variable region that is the product of or derived from a human germline gene. Accordingly, provided herein are isolated FIXz antibodies, or antigen-binding portions thereof, comprising a heavy chain variable region that is the product of or derived from a human VH germline gene selected from the group consisting of: VH1-18, VH1-46, VH3-21, VH3-30, VH4-31, VH4-39, VH4-0B, VH5-51, and any combination thereof. In particular embodiments, the VH germline gene is selected from the group consisting of VH1-18.0, VH1-18.1, VH1-18.8, VH1-46.0, VH1-46.4, VH1-46.5, VH1-46.6, VH1-46.7, VH1-46.8, VH1-46.9, VH3-21.0, VH3-23.0, VH3-23.2, VH3-23.6, VH3-30.0, VH4-31.5, VH4-39.0, VH4-39.5. VH4-0B.4, VH5-51.1, and any combination thereof.

In other embodiments, provided herein are isolated FIXa antibodies, or antigen-binding portions thereof, comprising a heavy chain variable region that is the product of or derived from a human VL germline gene selected from the group consisting of: VK1-05, VK1-12, VK1-39, VK2-28, VK3-11, VK3-15, VK3-20, VK4-01, and any combination thereof. In particular embodiments, the VL germline gene is selected from the group consisting of VK1-05.6, VK1-05.12, VK1-12.0, VK1-12.4, VK1-12.7, VK1-12.10, VK1-12.15, VK1-39.0, VK1-39.3, VK1-39.15, VK2-28.0, VK2-28.1, VK2-28.5, VK3-11.0, VK3-11.2, VK3-11.6, VK3-11.14, VK3-15.0, VK3-15.8, VK3-15.10, VK3-20.0, VK3-20.1, VK3-20.4, VK3-20.5, VK4-01.0, VK4-01.4, VK4-01.20, and any combination thereof.

In some aspects, the anti-FIXz antibody, or antigen binding portion thereof, comprises a VH and a VL, wherein the VH comprises an amino acid sequence at least 70%, at least 80%, at least 90%, at least 95%, at least 96%, at least 97%, at least 98%, at least 99%, or 100% identical to SEQ ID NO: 185 and VL comprises an amino acid sequence at least 70%, at least 80%, at least 90%, at least 95%, at least 96%, at least 97%, at least 98%, at least 99%, or 100% identical to SEQ ID NO: 371 (VH and VL of BIIB-9-578 antibody, respectively).

### (c) Anti-FXz Binding Molecules

The present disclosure also provides anti-FX binding molecules, e.g., anti-FX antibodies or molecules comprising FX-binding fragments thereof, that specifically bind FX. In some aspects of the present disclosure the disclosed anti-FX binding molecules, e.g., anti-FX antibodies or molecules comprising FX-binding fragments thereof, preferentially bind FX zymogen (FXz) (referred to throughout the present disclosure as “anti-FXz antibody”).

Factor X is a vitamin-K dependent glycoprotein with a molecular weight of 58.5 kDa, which is secreted from liver cells into the plasma as a zymogen. Initially factor X is produced as a prepropeptide with a signal peptide consisting in total of 488 amino acids. The amino acid sequence of FX zymogen (FXz) is provided below (the signal sequence (1-23) is underlined and the propeptide (24-40) is boldened):

(SEQ ID NO: 765)
MGRPLHLVLLSASLAGLLLLGESLFIRREQANNIL
ARVTRANSFLEEMKKGHLERECMEETCSYEEAREV
FEDSDKTNEFWNKYKDGDQCETSPCQNQGKCKDGL
GEYTCTCLEGFEGKNCELFTRKLCSLDNGDCDQFC
HEEQNSVVCSCARGYTLADNGKACIPTGPYPCGKQ
TLERRKRSVAQATSSSGEAPDSITWKPYDAADLDP
TENPFDLLDFNQTQPERGDNNLTRIVGGQECKDGE
CPWQALLINEENEGFCGGTILSEFYILTAAHCLYQ
AKRFKVRVGDRNTEQEEGGEAVHEVEVVIKHNRFT
KETYDFDIAVLRLKTPITFRMNVAPACLPERDWAE
STLMTQKTGIVSGFGRTHEKGRQSTRLKMLEVPYV
DRNSCKLSSSFIITQNMFCAGYDTKQEDACQGDSG
GPHVTRFKDTYFVTGIVSWGEGCARKGKYGIYTKV
TAFLKWIDRSMKTRGLPKAKSHAPEVITSSPLK

The signal peptide is cleaved off by signal peptidase during export into the endoplasmic reticulum. The propeptide sequence is cleaved off after gamma carboxylation took place at the first 11 glutamic acid residues at the N-terminus of the mature N-terminal chain. A further processing step occurs by cleavage between Arg182 and Ser183. This processing step also leads concomitantly to the deletion of the tripeptide Arg180-Lys181-Arg182. The resulting secreted factor X zymogen consists of an N-terminal light chain of 139 amino acids (M, 16,200) (i.e., amino acids 41 to 179 of SEQ ID NO: 765) and a C-terminal heavy chain of 306 amino acids (M, 42,000) (i.e., amino acids 183 to 488 of SEQ ID NO: 765), which are covalently linked via a disulfide bridge between Cys172 and Cys342. Further posttranslational processing steps include the β-hydroxylation of Asp103 as well as N- and O-type glycosylation.

FX zymogen can be cleaved in its heavy chain between Arg234 and Ile235 (corresponding to SEQ ID NO: 765) by factor IXa and consequently become activated after the release of an activation peptide.

The term “FX zymogen” can be used interchangeably herein with “FXz,” “FX precursor,” “unactivated FX,” “non-activated FX,” or “non-activated FX precursor.” In one embodiment, FX zymogen (FIXz) includes non-activated FX precursor in which the activation peptide (e.g., 52 amino acids activation peptide that are represented as amino acids 183 to 234 of SEQ ID NO: 765 (mature numbering) is not cleaved from the precursor. FIX zymogen can include any naturally-occurring or engineered variants. A non-limiting example of FX zymogen is shown in SEQ ID NO: 765. In another embodiment, FX zymogen is non-activatable FX (FXn), which is engineered to be non-active in the presence of Factor FIXa. An example of non-activatable FX can be FX carrying an arginine to alanine mutation at position 194 (mature numbering) preventing its activation and maintaining Factor X in the zymogen form (FXz). FX zymogen can optionally contain a signal peptide and/or propeptide.

The term “activated FX” can be used interchangeably herein with “FXa”. In one embodiment, activated FX is wild-type, naturally occurring FXa (also referred to herein as “wild-type FXa”). In another embodiment, FXa comprises non-naturally occurring FXa, e.g., FXa conformational variant. For example, FXa can be a FXa-SM, which is designed to have a conformation similar to the substrate bound FXa. In a particular embodiment, FXa-SM is activated FX with a substrate mimic covalently bound to the active site.

The present disclosure provides an antibody (e.g., an isolated antibody), or an antigen binding portion thereof, that specifically binds to FXz, wherein the anti-FX antibody or antigen binding portion thereof preferentially binds to FXz in the presence of FXz and FXa. In one embodiment, the FXa is FXa covalently attached to a substrate mimic (i.e., Glu-Gly-Arg-chloromethyl ketone (EGR-CMK)).

In some aspects, the anti-FXz antibody, or antigen binding portion thereof, binds to FXz with a binding affinity higher than a binding affinity of the antibody or antigen binding portion thereof to FXa. The present disclosure also provides an isolated anti-FX antibody, or antigen binding portion thereof, which binds to FXz with a binding affinity higher than a binding affinity of the antibody or antigen binding portion thereof to FXa.

In some aspects, the anti-FXz antibody, or antigen binding portion thereof, binds to FXz with a K_{D} of about 100 nM or less, about 95 nM or less, about 90 nM or less, about 85 nM or less, about 80 nM or less, about 75 nM or less, about 70 nM or less, about 65 nM or less, about 60 nM or less, about 55 nM or less, about 50 nM or less, about 45 nM or less, about 40 nM or less, about 35 nM or less, about 30 nM or less, about 25 nM or less, about 20 nM or less, about 15 nM or less, about 10 nM or less, about 5 nM or less, or about 1 nM or less as determined by a BLI assay.

In other embodiments, the anti-FXz antibody, or antigen binding portion thereof, binds to FXz with a K_{D} of about 10 nM or less, about 9 nM or less, about 8 nM or less, about 7 nM or less, about 6 nM or less, about 5 nM or less, about 4 nM or less, about 3 nM or less, about 2 nM or less, about 1 nM or less, about 0.5 nM or less, about 0.2 nM or less, about 0.1 nM or less, or about 0.05 nM or less. In yet other embodiments, the anti-FXz antibody, or antigen binding portion thereof, binds to FXz with a K_{D} of 1 nM to 100 nM, 1 nM to 90 nM, 1 nM to 80 nM, 1 nM to 70 nM, 1 nM to 60 nM, 1 nM to 50 nM, 1 nM to 40 nM, 1 nM to 30 nM, 1 nM to 20 nM, 1 nM to 10 nM, 0.1 nM to 100 nM, 0.1 nM to 90 nM, 0.1 nM to 80 nM, 0.1 nM to 70 nM, 0.1 nM to 60 nM, 0.1 nM to 50 nM, 0.1 nM to 40 nM, 0.1 nM to 30 nM, 0.1 nM to 20 nM, 0.1 nM to 10 nM, or 0.1 nM to 1 nM.

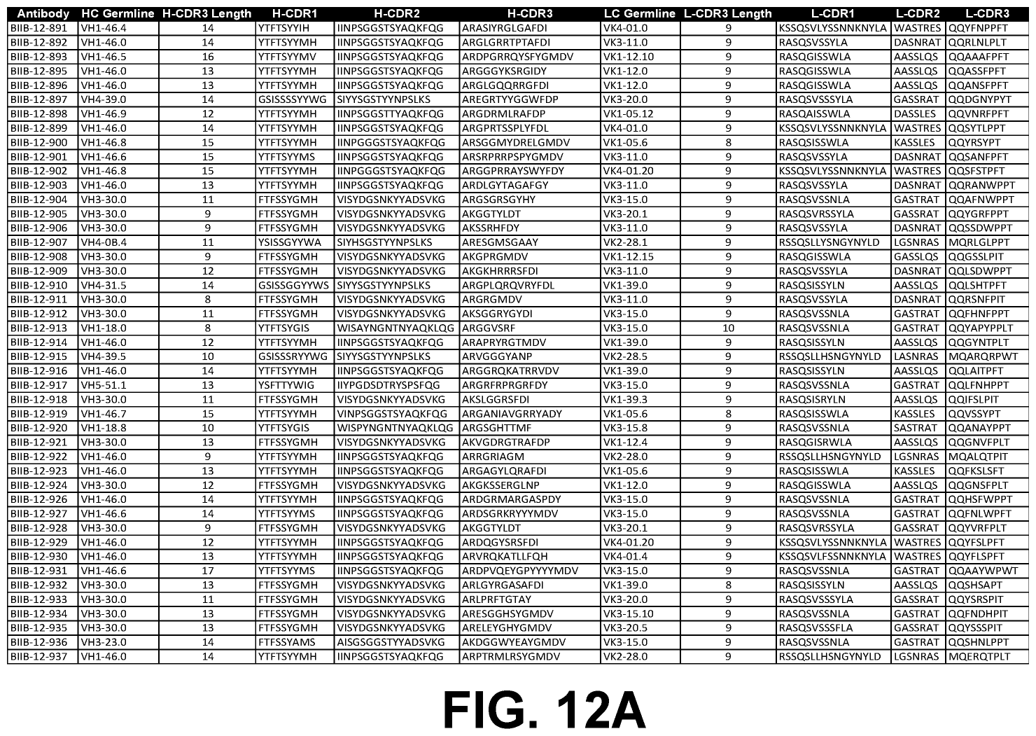

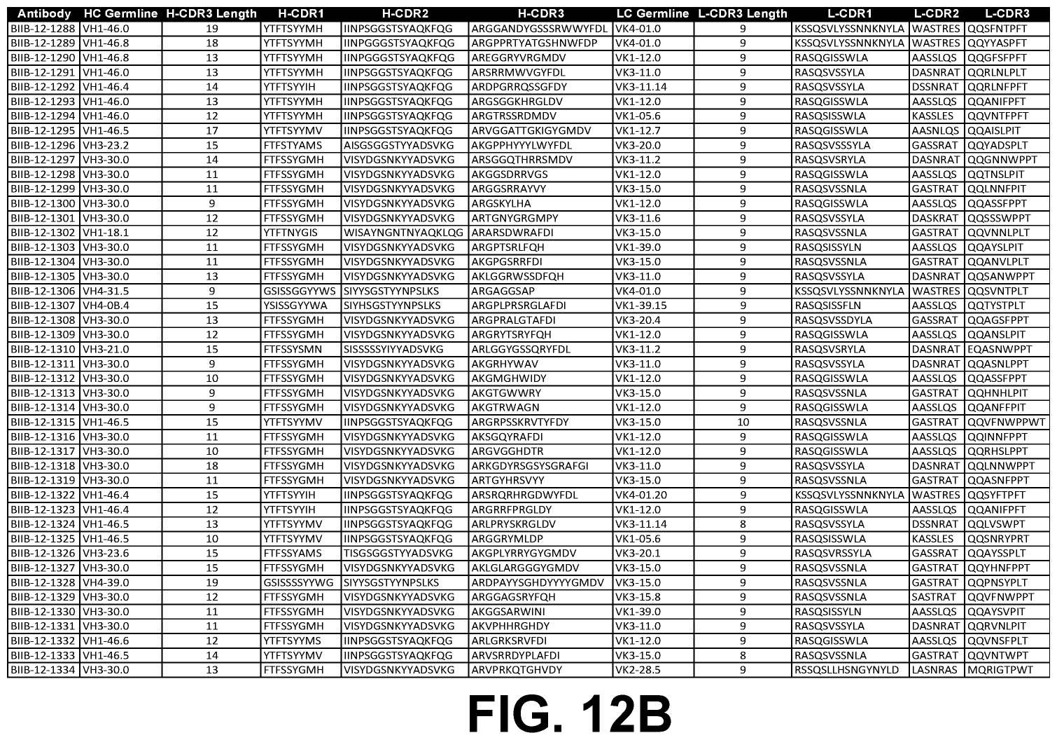

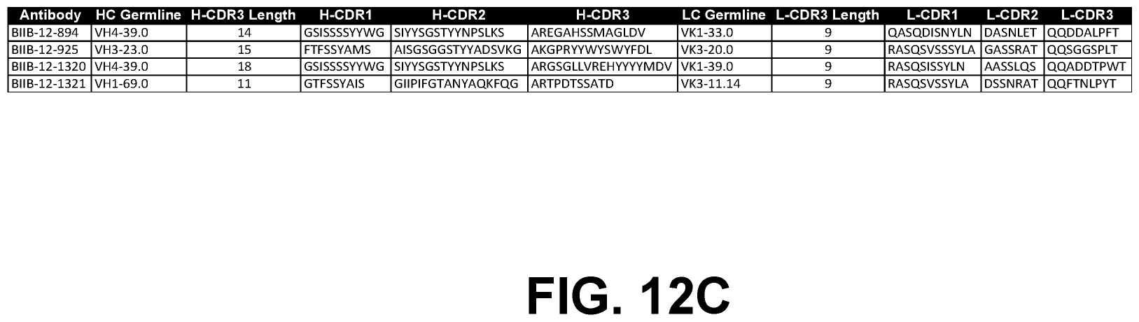

In some aspects, the anti-FXz antibody or an antigen binding portion thereof, cross-competes with a reference antibody selected from the group consisting of the antibodies in FIG. 12A and FIG. 12B. In some aspects, the anti-FXz antibody or antigen binding portion thereof, binds to the same epitope as a reference antibody selected from the group consisting of the antibodies in FIG. 12A and FIG. 12B. In some aspects, the anti-FXz or an antigen binding portion thereof, binds to the same epitope as a reference antibody selected from the group consisting of BIIB-12-915, BIIB-12-917, BIIB-12-932, and any combination thereof.

In some aspects, the anti-FXz antibody or an antigen binding portion thereof, binds to an antigen binding site (e.g., an epitope) that is substantially the same as the antigen binding site of any anti-FXz antibody or antigen binding portion thereof disclosed herein. In some aspects, the anti-FXz antibody or antigen binding portion thereof, binds to an antigen binding site (e.g., an epitope) that overlaps with the antigen binding site (e.g., epitope) of an anti-FXz antibody or antigen binding portion thereof disclosed herein.

In some aspects, the anti-FXz antibody or antigen binding portion thereof, comprises a VH CDR1, a VH CDR2, and a VH CDR3, wherein the VH CDR3 sequence comprises a
(i) a VH CDR3 sequence identical to a VH CDR3 sequence selected from the group consisting of VH CDR3 sequences in FIG. 12A or FIG. 12B; or,
(ii) a VH CDR3 sequence identical to a VH CDR3 sequence selected from the group consisting of VH CDR3 sequences in FIG. 12A or FIG. 12B except for 1, 2, or 3 amino acid substitutions.

In some aspects, the amino acid substitutions are conservative amino acid substitutions or back mutations

In some aspects, the anti-FXz antibody or antigen binding portion thereof, comprises VH CDR1, a VH CDR2, and a VH CDR3, wherein the VH CDR3 sequence comprises the amino acid sequence ARX₁X₂X₃RX₄X₅X₆X₇FDX₈ (SEQ ID NO: 766), wherein X₁ is G or L, X₂ is R or G, X₃ is F or Y, X₄ is P or G, X₅ is R or A, X₆ is G or S, X₇ is R or A, and X₈ is Y or I.

In some aspects, the anti-FXz antibody or antigen binding portion thereof, comprises VH CDR1, a VH CDR2, and a VH CDR3, wherein the VH CDR3 sequence consists or consists essentially of the amino acid sequence ARX₁X₂X₃RX₄X₅X₆X₇FDX₈ (SEQ ID NO: 766), wherein X₁ is G or L, X₂ is R or G, X₃ is F or Y, X₄ is P or G, X₅ is R or A, X₆ is G or S, X₇ is R or A, and X₈ is Y or I.

In some aspects, the anti-FXz antibody or antigen binding portion thereof, comprises a VH CDR1, a VH CDR2, and a VH CDR3, wherein the VH CDR3 sequence comprises an amino acid sequence selected from ARGRFRPRGRFDY (SEQ ID NO: 1575, BIIB-12-917), ARLGYRGASAFDI (SEQ ID NO: 1589, BIIB-12-932), or ARVGGGYANP (SEQ ID NO: 1573, BIIB-12-915).

In some aspects, the anti-FXz antibody, or antigen binding portion thereof, comprises a VH CDR1, a VH CDR2, and VH CDR3, wherein the VH CDR1 sequence comprises
(i) a VH CDR1 sequence identical to a sequence selected from the group consisting of the VH CDR1 sequences disclosed in FIG. 12A or FIG. 12B, or
(ii) a VH CDR1 sequence identical to a sequence selected from the group consisting of the VH CDR1 sequences disclosed in FIG. 12A or FIG. 12B except for 1, 2, or 3 amino acid substitutions.

In some aspects, the anti-FXz antibody, or antigen binding portion thereof, comprises a VH CDR1, a VH CDR2, and a VH CDR3, wherein the VH CDR2 sequence comprises
(i) a VH CDR2 sequence identical to a sequence selected from the group consisting of the VH CDR2 sequences disclosed in FIG. 12A or FIG. 12B, or
(ii) a VH CDR2 sequence identical to a sequence selected from the group consisting of the VH CDR2 sequences disclosed in FIG. 12A or FIG. 12B except for 1, 2, or 3 amino acid substitutions.

In some aspects, the anti-FXz antibody or antigen binding portion thereof, comprises a VL CDR1, a VL CDR2, and a VL CDR3, wherein the VL CDR1 sequence comprises
(i) a VL CDR1 sequence identical to a sequence selected from the group consisting of the VL CDR1 sequences disclosed in FIG. 12A or FIG. 12B, or
(ii) a VL CDR1 sequence identical to a sequence selected from the group consisting of the VL CDR1 sequences disclosed in FIG. 12A or FIG. 12B except for 1, 2, or 3 amino acid substitutions.

In some aspects, the anti-FXz antibody or antigen binding portion thereof, comprises a VL CDR1, a VL CDR2, and a VL CDR3, wherein the VL CDR2 sequence comprises
(i) a VL CDR2 sequence identical to a sequence selected from the group consisting of the VL CDR2 sequences in FIG. 12A or FIG. 12B, or
(ii) a VL CDR2 sequence identical to a sequence selected from the group consisting of the VL CDR2 sequences in FIG. 12A or FIG. 12B except for 1, 2, or 3 amino acid substitutions.

In some aspects, the anti-FXz antibody or antigen binding portion thereof, comprises a VL CDR1, a VL CDR2, and a VL CDR3, wherein the VL CDR3 sequence comprises
(i) a VL CDR3 sequence identical to a sequence selected from the group consisting of the VL CDR3 sequences disclosed in FIG. 12A or FIG. 12B, or
(ii) a VL CDR3 sequence identical to a sequence selected from the group consisting of the VL CDR3 sequences disclosed in FIG. 12A or FIG. 12B except for 1, 2, or 3 amino acid substitutions.

In some aspects, the anti-FXz antibody or antigen binding portion thereof, comprises a VL CDR1, a VL CDR2, and a VL CDR3, wherein the VL CDR3 sequence consists or consists essentially of
(i) a VL CDR3 sequence identical to a sequence selected from the group consisting of the VL CDR3 sequences disclosed in FIG. 12A or FIG. 12B, or
(ii) a VL CDR3 sequence identical to a sequence selected from the group consisting of the VL CDR3 sequences disclosed in FIG. 12A or FIG. 12B except for 1, 2, or 3 amino acid substitutions.

The present disclosure also provides an isolated antibody, or antigen binding portion thereof, which specifically binds to FXz, comprising a VH CDR1, a VH CDR2, and a VH CDR3 and a VL CDR1, a VL CDR2, and a VL CDR3, wherein the VH CDR1, VH CDR2, and VH CDR3 and the VL CDR1, VL CDR2, and VL CDR3 comprise the VH CDR1, VH CDR2, and VH CDR3 and the VL CDR1, VL CDR2, and VL CDR3 of an anti-FXz antibody selected from the group consisting of BIIB-12-891, BIIB-12-892, BIIB-12-893, BIIB-12-895, BIIB-12-896, BIIB-12-897, BIIB-12-898, BIIB-12-899, BIIB-12-900, BIIB-12-901, BIIB-12-902, BIIB-12-903, BIIB-12-904, BIIB-12-905, BIIB-12-906, BIIB-12-907, BIIB-12-908, BIIB-12-909, BIIB-12-910, BIIB-12-911, BIIB-12-912, BIIB-12-913, BIIB-12-914, BIIB-12-915, BIIB-12-916, BIIB-12-917, BIIB-12-918, BIIB-12-919, BIIB-12-920, BIIB-12-921, BIIB-12-922, BIIB-12-923, BIIB-12-924, BIIB-12-926, BIIB-12-927, BIIB-12-928, BIIB-12-929, BIIB-12-930, BIIB-12-931, BIIB-12-932, BIIB-12-933, BIIB-12-934, BIIB-12-935, BIIB-12-936, BIIB-12-937, BIIB-12-1288, BIIB-12-1289, BIIB-12-1290, BIIB-12-1291, BIIB-12-1292, BIIB-12-1293, BIIB-12-1294, BIIB-12-1295, BIIB-12-1296, BIIB-12-1297, BIIB-12-1298, BIIB-12-1299, BIIB-12-1300, BIIB-12-1301, BIIB-12-1302, BIIB-12-1303, BIIB-12-1304, BIIB-12-1305, BIIB-12-1306, BIIB-12-1307, BIIB-12-1308, BIIB-12-1309, BIIB-12-1310, BIIB-12-1311, BIIB-12-1312, BIIB-12-1313, BIIB-12-1314, BIIB-12-1315, BIIB-12-1316, BIIB-12-1317, BIIB-12-1318, BIIB-12-1319, BIIB-12-1322, BIIB-12-1323, BIIB-12-1324, BIIB-12-1325, BIIB-12-1326, BIIB-12-1327, BIIB-12-1328, BIIB-12-1329, BIIB-12-1330, BIIB-12-1331, BIIB-12-1332, BIIB-12-1333, or BIIB-12-1334.

In some aspects, the anti-FXz antibody, or antigen binding portion thereof, comprises VH CDR1, VH CDR2, and VH CDR3 sequences comprising SEQ ID NO: 1393, SEQ ID NO: 1483, and SEQ ID NO: 1573, respectively (VH CDRs of BIIB-12-915 antibody), and/or VL CDR1, VL CDR2, and VL CDR3 sequences comprising SEQ ID NO: 1663, SEQ ID NO: 1753, and SEQ ID NO: 1843, respectively (VL CDRs of BIIB-12-915 antibody).

In some aspects, the anti-FXz antibody, or antigen binding portion thereof, comprises VH CDR1, VH CDR2, and VH CDR3 sequences comprising SEQ ID NO: 1395, SEQ ID NO: 1485, and SEQ ID NO: 1575, respectively (VH CDRs of BIIB-12-917 antibody), and/or VL CDR1, VL CDR2, and VL CDR3 sequences comprising SEQ ID NO: 1665, SEQ ID NO: 1755, and SEQ ID NO: 1845, respectively (VL CDRs of BIIB-12-917 antibody).

In some aspects, the anti-FXz antibody, or antigen binding portion thereof, comprises VH CDR1, VH CDR2, and VH CDR3 sequences comprising SEQ ID NO: 1409, SEQ ID NO: 1499, and SEQ ID NO: 1589, respectively (VH CDRs of BIIB-12-932 antibody), and/or VL CDR1, VL CDR2, and VL CDR3 sequences comprising SEQ ID NO: 1679, SEQ ID NO: 1769, and SEQ ID NO: 1859, respectively (VL CDRs of BIIB-12-932 antibody).

In some aspects, the anti-FXz antibody or antigen binding portion thereof, comprises a VH and a VL, wherein the VH comprises an amino acid sequence which is at least about 70%, at least about 75%, at least about 80%, at least about 85%, at least about 90%, at least about 95%, at least about 96%, at least about 97%, at least about 98%, at least about 99%, or about 100% identical to the amino acid sequence selected from the group consisting of SEQ ID NOs: 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, and 555.

In some aspects, the anti-FXz antibody or antigen binding portion thereof, comprises a VH and a VL, wherein the VL comprises an amino acid sequence which is at least about 70%, at least about 75%, at least about 80%, at least about 85%, at least about 90%, at least about 95%, at least about 96%, at least about 97%, at least about 98%, at least about 99%, or about 100% identical to the amino acid sequence selected from the group consisting of SEQ ID NOs: 565, 567, 569, 571, 573, 575, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, and 743.

In some aspects, the anti-FXz antibody or antigen binding portion thereof, comprises a VH and a VL, wherein

(i) the VH comprises an amino acid sequence which is at least about 70%, at least about 75%, at least about 80%, at least about 85%, at least about 90%, at least about 95%, at least about 96%, at least about 97%, at least about 98%, at least about 99%, or about 100% identical to the amino acid sequence selected from the group consisting of SEQ ID NOs: 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, and 555; and
(ii) the VL comprises an amino acid sequence which is at least about 70%, at least about 75%, at least about 80%, at least about 85%, at least about 90%, at least about 95%, at least about 96%, at least about 97%, at least about 98%, at least about 99%, or about 100% identical to the amino acid sequence selected from the group consisting of 565, 567, 569, 571, 573, 575, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, and 743.

In certain embodiments, an anti-FIXa antibody comprises a heavy chain variable region from a particular germline heavy chain immunoglobulin gene and/or a light chain variable region from a particular germline light chain immunoglobulin gene. In some embodiments, the VH sequence of the anti-FIXa antibody can be derived from any one of V, D, or J germline sequences and/or the VL sequence of the anti-FIXa antibody can be derived from any one of kappa or lambda germline sequence.

As demonstrated herein, human antibodies specific for FIXa have been prepared that comprise a heavy chain variable region that is the product of or derived from a human germline gene. Accordingly, provided is an anti-FXz antibody or antigen binding portion thereof, comprising a VH and a VL, wherein the VH is derived from a germline sequence of VH1-18, VH1-46, VH3-21, VH3-23, VH3-30, VH4-31, VH4-39, VH4-0B, or VH5-51. In some aspects, the anti-FXz antibody or antigen binding portion thereof, comprises a VH and a VL, wherein the VL is derived from a germline sequence of VK1-05, VK1-12, VK1-39, VK2-28, VK3-11, VK3-15, VK3-20, or VK4-01. In some aspects, the anti-FXz antibody or antigen binding portion thereof, comprises a VH and a VL, wherein the VH is derived from a germline sequence of VH1-18.0, VH1-18.1, VH1-18.8, VH1-46.0, VH1-46.4, VH1-46.5, VH1-46.6, VH1-46.7, VH1-46.8, VH1-46.9, VH3-21.0, VH3-23.0, VH3-23.2, VH3-23.6, VH3-30.0, VH4-31.5, VH4-39.0, VH4-39.5. VH4-0B.4, or VH5-51.1, and the VL is derived from a germline sequence of VK1-05.6, VK1-05.12, VK1-12.0, VK1-12.4, VK1-12.7, VK1-12.10, VK1-12.15, VK1-39.0, VK1-39.3, VK1-39.15, VK2-28.0, VK2-28.1, VK2-28.5, VK3-11.0, VK3-11.2, VK3-11.6, VK3-11.14, VK3-15.0, VK3-15.8, VK3-15.10, VK3-20.0, VK3-20.1, VK3-20.4, VK3-20.5, VK4-01.0, VK4-01.4, VK4-01.20. In some aspects, the VH and/or the VL are derived from their respective germlines via affinity optimization

Antibodies described herein include those comprising a heavy chain variable region that is the product of or derived from one of the above-listed human germline VH genes and also comprising a light chain variable region that is the product of or derived from one of the above-listed human germline VK genes, as shown in the Figures.

As used herein, a human antibody comprises heavy and light chain variable regions that are “the product of or “derived from” a particular germline sequence if the variable regions of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody that is “the product of or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “the product of” or “derived from” a particular human germline immunoglobulin sequence can contain amino acid differences as compared to the germline sequence, due to, for example, naturally-occurring somatic mutations or intentional introduction of site-directed mutation. However, a selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody can be at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody can display no more than 5, or even no more than 4, 3, 2, or 1 amino acid difference from the amino acid sequence encoded by the germline immunoglobulin gene.

In some aspects, the anti-FXz antibody or antigen binding portion thereof comprises at least one VH, wherein the VH comprises, consists, or consists essentially of a sequence selected from SEQ ID NOS: 423, 427, or 455.

In some aspects, the anti-FXz antibody or antigen binding portion thereof comprises at least one VL, wherein the VL comprises, consists, or consists essentially of a sequence selected from SEQ ID NOS: 611, 615, or 643.

In some aspects, the anti-FXz antibody or antigen binding portion thereof comprises at least one VH and at least one VL, wherein
(i) at least one VH comprises, consists, or consists essentially of a sequence selected from SEQ ID NOS: 423, 427, or 455; and,
(ii) at least one VL comprises, consists, or consists essentially of a sequence selected from SEQ ID NOS: 611, 615, or 643.

In some aspects, the anti-FXz antibody or antigen binding portion thereof, comprises a VH and a VL, wherein

(b1) VH and VL comprise, consist, or consist essentially of SEQ ID NOs: 423 and 611, respectively;
(b2) VH and VL comprise, consist, or consist essentially of SEQ ID NOs: 427 and 615, respectively; or
(b3) VH and VL comprise, consist, or consist essentially of SEQ ID NOs: 455 and 643, respectively.

In some aspects, the anti-FXz antibody, or antigen binding portion thereof, comprises a VH and a VL, wherein the VH comprises an amino acid sequence at least 70%, at least 80%, at least 90%, at least 95%, at least 96%, at least 97%, at least 98%, at least 99%, or 100% identical to SEQ ID NO: 423 and VL comprises an amino acid sequence at least 70%, at least 80%, at least 90%, at least 95%, at least 96%, at least 97%, at least 98%, at least 99%, or 100% identical to SEQ ID NO: 611.

In some aspects, the anti-FXz antibody, or antigen binding portion thereof, comprises a VH and a VL, wherein the VH comprises an amino acid sequence at least 70%, at least 80%, at least 90%, at least 95%, at least 96%, at least 97%, at least 98%, at least 99%, or 100% identical to SEQ ID NO: 427 and VL comprises an amino acid sequence at least 70%, at least 80%, at least 90%, at least 95%, at least 96%, at least 97%, at least 98%, at least 99%, or 100% identical to SEQ ID NO: 615.

In some aspects, the anti-FXz antibody, or antigen binding portion thereof, comprises a VH and a VL, wherein the VH comprises an amino acid sequence at least 70%, at least 80%, at least 90%, at least 95%, at least 96%, at least 97%, at least 98%, at least 99%, or 100% identical to SEQ ID NO: 455 and VL comprises an amino acid sequence at least 70%, at least 80%, at least 90%, at least 95%, at least 96%, at least 97%, at least 98%, at least 99%, or 100% identical to SEQ ID NO: 643.

In certain aspects, the present anti-FXz antibody, or antigen binding portion thereof, has no detectable binding to FXa. In other aspects, a bispecific molecule of the disclosure comprises an anti-FXz antibody, or antigen binding portion thereof, that specifically binds to FXz and has no detectable binding to FXa and an anti-FIX antibody that specifically binds to both FIXz and FIXa.

### (d) Anti-FXa Binding Molecules

The present disclosure provides an antibody (e.g., an isolated antibody), or an antigen binding portion thereof, that specifically binds to activated factor X (FXa), wherein the anti-FX antibody or antigen binding portion thereof preferentially binds to FXa in the presence of FXz and FXa. In one embodiment, the FXa is FXa covalently attached to a substrate mimic (i.e., EGR-CMK).

In some aspects, the anti-FXa antibody, or antigen binding portion thereof, binds to FXa with a binding affinity higher than a binding affinity of the antibody or antigen binding portion thereof to FXz. The present disclosure also provides an isolated anti-FX antibody, or antigen binding portion thereof, which binds to FXa with a binding affinity higher than a binding affinity of the antibody or antigen binding portion thereof to FXz.

In some aspects, the anti-FXa antibody, or antigen binding portion thereof, binds to FXa with a K_{D} of about 100 nM or less, about 95 nM or less, about 90 nM or less, about 85 nM or less, about 80 nM or less, about 75 nM or less, about 70 nM or less, about 65 nM or less, about 60 nM or less, about 55 nM or less, about 50 nM or less, about 45 nM or less, about 40 nM or less, about 35 nM or less, about 30 nM or less, about 25 nM or less, about 20 nM or less, about 15 nM or less, about 10 nM or less, about 5 nM or less, or about 1 nM or less as determined by a BLI assay.

In other embodiments, the anti-FXa antibody, or antigen binding portion thereof, binds to FXa with a K_{D} of about 10 nM or less, about 9 nM or less, about 8 nM or less, about 7 nM or less, about 6 nM or less, about 5 nM or less, about 4 nM or less, about 3 nM or less, about 2 nM or less, about 1 nM or less, about 0.5 nM or less, about 0.2 nM or less, about 0.1 nM or less, or about 0.05 nM or less. In yet other embodiments, the anti-FXa antibody, or antigen binding portion thereof, binds to FXa with a K_{D} of 1 nM to 100 nM, 1 nM to 90 nM, 1 nM to 80 nM, 1 nM to 70 nM, 1 nM to 60 nM, 1 nM to 50 nM, 1 nM to 40 nM, 1 nM to 30 nM, 1 nM to 20 nM, 1 nM to 10 nM, 0.1 nM to 100 nM, 0.1 nM to 90 nM, 0.1 nM to 80 nM, 0.1 nM to 70 nM, 0.1 nM to 60 nM, 0.1 nM to 50 nM, 0.1 nM to 40 nM, 0.1 nM to 30 nM, 0.1 nM to 20 nM, 0.1 nM to 10 nM, or 0.1 nM to 1 nM.

In some aspects, the anti-FXa antibody or an antigen binding portion thereof, cross-competes with a reference antibody selected from the group consisting of the antibodies in FIG. 12C. In some aspects, the anti-FXa antibody or antigen binding portion thereof, binds to the same epitope as a reference antibody selected from the group consisting of the antibodies in FIG. 12C. In some aspects, the anti-FXa or an antigen binding portion thereof, binds to the same epitope as a reference antibody, which is BIIB-12-925.

In some aspects, the anti-FXa antibody or an antigen binding portion thereof, binds to an antigen binding site (e.g., an epitope) that is substantially the same as the antigen binding site of any anti-FXa antibody or antigen binding portion thereof disclosed herein. In some aspects, the anti-FXa antibody or antigen binding portion thereof, binds to an antigen binding site (e.g., an epitope) that overlaps with the antigen binding site (e.g., epitope) of an anti-FXa antibody or antigen binding portion thereof disclosed herein.

In some aspects, the anti-FXa antibody or antigen binding portion thereof, comprises a VH CDR1, a VH CDR2, and a VH CDR3, wherein the VH CDR3 sequence comprises a
(i) a VH CDR3 sequence identical to a VH CDR3 sequence selected from the group consisting of VH CDR3 sequences in FIG. 12C; or,
(ii) a VH CDR3 sequence identical to a VH CDR3 sequence selected from the group consisting of VH CDR3 sequences in FIG. 12C except for 1, 2, or 3 amino acid substitutions.

In some aspects, the amino acid substitutions are conservative amino acid substitutions or back mutations

In some aspects, the anti-FXa antibody or antigen binding portion thereof, comprises a VH CDR1, a VH CDR2, and a VH CDR3, wherein the VH CDR3 sequence comprises an amino acid sequence set forth as (SEQ ID NO: 1919, BIIB-12-925).

In some aspects, the anti-FXa antibody, or antigen binding portion thereof, comprises a VH CDR1, a VH CDR2, and VH CDR3, wherein the VH CDR1 sequence comprises
(i) a VH CDR1 sequence identical to a sequence selected from the group consisting of the VH CDR1 sequences disclosed in FIG. 12C, or
(ii) a VH CDR1 sequence identical to a sequence selected from the group consisting of the VH CDR1 sequences disclosed in FIG. 12C except for 1, 2, or 3 amino acid substitutions.

In some aspects, the anti-FXa antibody, or antigen binding portion thereof, comprises a VH CDR1, a VH CDR2, and a VH CDR3, wherein the VH CDR2 sequence comprises
(i) a VH CDR2 sequence identical to a sequence selected from the group consisting of the VH CDR2 sequences disclosed in FIG. 12C, or
(ii) a VH CDR2 sequence identical to a sequence selected from the group consisting of the VH CDR2 sequences disclosed in FIG. 12C except for 1, 2, or 3 amino acid substitutions.

In some aspects, the anti-FXa antibody or antigen binding portion thereof, comprises a VL CDR1, a VL CDR2, and a VL CDR3, wherein the VL CDR1 sequence comprises
(i) a VL CDR1 sequence identical to a sequence selected from the group consisting of the VL CDR1 sequences disclosed in FIG. 12C, or
(ii) a VL CDR1 sequence identical to a sequence selected from the group consisting of the VL CDR1 sequences disclosed in FIG. 12C except for 1, 2, or 3 amino acid substitutions.

In some aspects, the anti-FXa antibody or antigen binding portion thereof, comprises a VL CDR1, a VL CDR2, and a VL CDR3, wherein the VL CDR2 sequence comprises
(i) a VL CDR2 sequence identical to a sequence selected from the group consisting of the VL CDR2 sequences in FIG. 12C, or
(ii) a VL CDR2 sequence identical to a sequence selected from the group consisting of the VL CDR2 sequences in FIG. 12C except for 1, 2, or 3 amino acid substitutions.

In some aspects, the anti-FXa antibody or antigen binding portion thereof, comprises a VL CDR1, a VL CDR2, and a VL CDR3, wherein the VL CDR3 sequence comprises
(i) a VL CDR3 sequence identical to a sequence selected from the group consisting of the VL CDR3 sequences disclosed in FIG. 12C, or
(ii) a VL CDR3 sequence identical to a sequence selected from the group consisting of the VL CDR3 sequences disclosed in FIG. 12C except for 1, 2, or 3 amino acid substitutions.

In some aspects, the anti-FXa antibody or antigen binding portion thereof, comprises a VL CDR1, a VL CDR2, and a VL CDR3, wherein the VL CDR3 sequence consists or consists essentially of
(i) a VL CDR3 sequence identical to a sequence selected from the group consisting of the VL CDR3 sequences disclosed in FIG. 12C, or
(ii) a VL CDR3 sequence identical to a sequence selected from the group consisting of the VL CDR3 sequences disclosed in FIG. 12C except for 1, 2, or 3 amino acid substitutions.

The present disclosure also provides an isolated antibody, or antigen binding portion thereof, which specifically binds to FXa, comprising a VH CDR1, a VH CDR2, and a VH CDR3 and a VL CDR1, a VL CDR2, and a VL CDR3, wherein the VH CDR1, VH CDR2, and VH CDR3 and the VL CDR1, VL CDR2, and VL CDR3 comprise the VH CDR1, VH CDR2, and VH CDR3 and the VL CDR1, VL CDR2, and VL CDR3 of an anti-FXa antibody selected from the group consisting of BIIB-12-894, BIIB-12-925, BIIB-12-1320, or BIIB-12-1321.

In some aspects, the anti-FXa antibody, or antigen binding portion thereof, comprises VH CDR1, VH CDR2, and VH CDR3 sequences comprising SEQ ID NO: 1911, SEQ ID NO: 1915, and SEQ ID NO: 1919, respectively, and/or VL CDR1, VL CDR2, and VL CDR3 sequences comprising SEQ ID NO: 1923, SEQ ID NO: 1927, and SEQ ID NO: 1931, respectively.

In some aspects, the anti-FXa antibody or antigen binding portion thereof, comprises a VH and a VL, wherein the VH comprises an amino acid sequence which is at least about 70%, at least about 75%, at least about 80%, at least about 85%, at least about 90%, at least about 95%, at least about 96%, at least about 97%, at least about 98%, at least about 99%, or about 100% identical to the amino acid sequence selected from the group consisting of SEQ ID NOs: 557, 559, 561, and 563.

In some aspects, the anti-FXa antibody or antigen binding portion thereof, comprises a VH and a VL, wherein the VL comprises an amino acid sequence which is at least about 70%, at least about 75%, at least about 80%, at least about 85%, at least about 90%, at least about 95%, at least about 96%, at least about 97%, at least about 98%, at least about 99%, or about 100% identical to the amino acid sequence selected from the group consisting of SEQ ID NOs: 745, 747, 749, and 751.

In some aspects, the anti-FXa antibody or antigen binding portion thereof, comprises a VH and a VL, wherein

(i) the VH comprises an amino acid sequence which is at least about 70%, at least about 75%, at least about 80%, at least about 85%, at least about 90%, at least about 95%, at least about 96%, at least about 97%, at least about 98%, at least about 99%, or about 100% identical to the amino acid sequence selected from the group consisting of SEQ ID NOs: 557, 559, 561, and 563; and,
(ii) the VL comprises an amino acid sequence which is at least about 70%, at least about 75%, at least about 80%, at least about 85%, at least about 90%, at least about 95%, at least about 96%, at least about 97%, at least about 98%, at least about 99%, or about 100% identical to the amino acid sequence selected from the group consisting of SEQ ID NOs: 745, 747, 749, and 751.

In certain embodiments, an anti-FIXa antibody comprises a heavy chain variable region from a particular germline heavy chain immunoglobulin gene and/or a light chain variable region from a particular germline light chain immunoglobulin gene. In some embodiments, the VH sequence of the anti-FXa antibody can be derived from any one of V, D, or J germline sequences and/or the VL sequence of the anti-FXa antibody can be derived from any one of kappa or lambda germline sequence.

As demonstrated herein, human antibodies specific for FXa have been prepared that comprise a heavy chain variable region that is the product of or derived from a human germline gene. Accordingly, provided is an anti-FXa antibody or antigen binding portion thereof, comprising a VH and a VL, wherein the VH is derived from a germline sequence of VH1-18, VH1-46, VH3-21, VH3-23, VH3-30, VH4-31, VH4-39, VH4-0B, or VH5-51. In some aspects, the anti-FXa antibody or antigen binding portion thereof, comprises a VH and a VL, wherein the VL is derived from a germline sequence of VK1-05, VK1-12, VK1-39, VK2-28, VK3-11, VK3-15, VK3-20, or VK4-01. In some aspects, the anti-FXa antibody or antigen binding portion thereof, comprises a VH and a VL, wherein the VH is derived from a germline sequence of VH1-18.0, VH1-18.1, VH1-18.8, VH1-46.0, VH1-46.4, VH1-46.5, VH1-46.6, VH1-46.7, VH1-46.8, VH1-46.9, VH3-21.0, VH3-23.0, VH3-23.2, VH3-23.6, VH3-30.0, VH4-31.5, VH4-39.0, VH4-39.5. VH4-0B.4, or VH5-51.1, and the VL is derived from a germline sequence of VK1-05.6, VK1-05.12, VK1-12.0, VK1-12.4, VK1-12.7, VK1-12.10, VK1-12.15, VK1-39.0, VK1-39.3, VK1-39.15, VK2-28.0, VK2-28.1, VK2-28.5, VK3-11.0, VK3-11.2, VK3-11.6, VK3-11.14, VK3-15.0, VK3-15.8, VK3-15.10, VK3-20.0, VK3-20.1, VK3-20.4, VK3-20.5, VK4-01.0, VK4-01.4, VK4-01.20. In some aspects, the VH and/or the VL are derived from their respective germlines via affinity optimization

In some aspects, the anti-FXa antibody, or antigen binding portion thereof, comprises a VH and a VL, wherein the VH comprises an amino acid sequence at least 70%, at least 80%, at least 90%, at least 95%, at least 96%, at least 97%, at least 98%, at least 99%, or 100% identical to SEQ ID NO: 559 and VL comprises an amino acid sequence at least 70%, at least 80%, at least 90%, at least 95%, at least 96%, at least 97%, at least 98%, at least 99%, or 100% identical to SEQ ID NO: 747.

### (e) Common Aspects

Certain aspects of the present disclosure pertains to an anti-FIX antibody (e.g., anti-FIXa antibody or anti-FIXz antibody) and anti-FX antibody (e.g., anti-FXa antibody or anti-FXz antibody) disclosed herein comprising the VH and VL CDR sequences disclosed herein, yet containing different framework sequences from the antibodies disclosed herein. Such framework sequences can be obtained from public DNA databases or published references that include germline antibody gene sequences. For example, germline DNA sequences for human heavy and light chain variable region genes can be found in the “VBase” human germline sequence database (available on the Internet at www.mrc-cpe.cam.ac.uk/vbase), as well as in Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline VH Sequences Reveals about Fifty Groups of VH Segments with Different Hypervariable Loops” Mol. Biol. 227:776-798; and Cox, J. P. L. et al. (1994) “A Directory of Human Germ-line VH Segments Reveals a Strong Bias in their Usage” Eur. J. Immunol. 24:827-836; the contents of each of which are expressly incorporated herein by reference.

Exemplary framework sequences for use in the antibodies described herein are those that are structurally similar to the framework sequences used by antibodies described herein. The VH CDR1, 2 and 3 sequences, and the VL CDR1, 2 and 3 sequences, can be grafted onto framework regions that have the identical sequence as that found in the germline immunoglobulin gene from which the framework sequence derive, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al).

Engineered antibodies described herein include those in which modifications have been made to framework residues within VH and/or VL, e.g., to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis. Such “backmutated” antibodies are also intended to be encompassed. Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Patent Publication No. 20030153043 by Carr et al.

Another type of variable region modification is to mutate amino acid residues within the VH and/or VL CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding, or other functional property of interest, can be evaluated in in vitro or in vivo assays as described herein and provided in the Examples. In some embodiments, conservative modifications (as discussed above) are introduced. The mutations can be amino acid substitutions, additions or deletions. Moreover, typically no more than one, two, three, four or five residues within a CDR region are altered.

In some aspects, methionine residues in CDRs of antibodies can be oxidized, resulting in potential chemical degradation and consequent reduction in potency of the antibody. Accordingly, also provided are anti-FIX/FX antibodies which have one or more methionine residues in the heavy and/or light chain CDRs replaced with amino acid residues which do not undergo oxidative degradation. In one embodiment, the methionine residues in the CDRs of the antibodies disclosed herein are replaced with amino acid residues which do not undergo oxidative degradation. Similarly, deamidation sites may be removed from any of the antibodies, particularly in the CDRs.

In certain aspects, any one of the anti-FIX antibodies (e.g., anti-FIXa antibodies or anti-FIXz antibodies) and anti-FX antibodies (e.g., anti-FXa antibodies or anti-FXz antibodies) disclosed herein can be an IgG. In some aspects, the IgG is an IgG1, an IgG2, an IgG3, an IgG4 or a variant thereof. In some aspects, any one of the anti-FIX antibodies (e.g., anti-FIXa antibodies or anti-FIXz antibodies) and anti-FX antibodies (e.g., anti-FXa antibodies or anti-FXz antibodies) disclosed herein is an IgG4 antibody. In some aspects, an anti-FIX antibody (e.g., anti-FIXa antibody or anti-FIXz antibody) and anti-FX antibody (e.g., anti-FXa antibody or anti-FXz antibody) disclosed herein comprises an effectorless IgG4 Fc.

In some aspects, the heavy chain constant region or fragment thereof of an anti-FIX antibody (e.g., anti-FIXa antibody or anti-FIXz antibody) or anti-FX antibody (e.g., anti-FXa antibody or anti-FXz antibody) is an IgG constant region. In some aspects, the IgG constant region or fragment thereof is an IgG1, IgG2, IgG3, or IgG4 constant region. In some aspects, an anti-FIX antibody (e.g., anti-FIXa antibody or anti-FIXz antibody) or anti-FX antibody (e.g., anti-FXa antibody or anti-FXz antibody) comprises a VL comprising a light chain constant region (LC), wherein the LC constant region is a kappa constant region. In some aspects, an anti-FIX antibody (e.g., anti-FIXa antibody or anti-FIXz antibody) or anti-FX antibody (e.g., anti-FXa antibody or anti-FXz antibody) comprises a VL comprising a light chain constant region (LC), wherein the LC constant region is a lambda constant region.

In some aspects, an anti-FIX antibody (e.g., anti-FIXa antibody or anti-FIXz antibody) or anti-FX antibody (e.g., anti-FXa antibody or anti-FXz antibody) comprises a heavy chain constant region (CH). In some aspects, an anti-FIX antibody (e.g., anti-FIXa antibody or anti-FIXz antibody) or anti-FX antibody (e.g., anti-FXa antibody or anti-FXz antibody) comprises a CH1 domain, CH2 domain, or CH3 domain.

In some aspects, a FIX or FX antigen binding portion thereof comprises an Fab, Fab′, F(ab′)2, Fv, or a single chain Fv (scFv). In other aspects, a FIX or FX antigen binding portion thereof comprises an Fd, scFv, disulfide stabilized scFv, a disulfide linked Fv, a V-NAR domain, an IgNar, an intrabody, an IgG CH2, a minibody, a F(ab′)₃, a tetrabody, a triabody, a diabody, a single-domain antibody, DVD-Ig, Fcab, mAb², a (scFv)₂, or a scFv-Fc.

In some aspects, the anti-FIX antibody (e.g., anti-FIXa antibody or anti-FIXz antibody) or anti-FX antibody (e.g., anti-FXa antibody or anti-FXz antibody) disclosed herein are monospecific. In other aspects, the anti-FIX antibody (e.g., anti-FIXa antibody or anti-FIXz antibody) or anti-FX antibody (e.g., anti-FXa antibody or anti-FXz antibody) is bispecific, trispecific, tetraspecific, etc. In other aspects, the anti-FIX antibody (e.g., anti-FIXa antibody or anti-FIXz antibody) or anti-FX antibody (e.g., anti-FXa antibody or anti-FXz antibody) is multispecific. In some aspects, the anti-FIX antibody (e.g., anti-FIXa antibody or anti-FIXz antibody) or anti-FX antibody (e.g., anti-FXa antibody or anti-FXz antibody) is monovalent, bivalent, trivalent, tetravalent, etc. In yet other aspects, the anti-FIX antibody (e.g., anti-FIXa antibody or anti-FIXz antibody) or anti-FX antibody (e.g., anti-FXa antibody or anti-FXz antibody) is multivalent. In specific aspects, the anti-FIX antibody (e.g., anti-FIXa antibody or anti-FIXz antibody) or anti-FX antibody (e.g., anti-FXa antibody or anti-FXz antibody) is bivalent, e.g., an antibody comprising several specific antigen binding sites. In specific aspects, the anti-FIX antibody (e.g., anti-FIXa antibody or anti-FIXz antibody) or anti-FX antibody (e.g., anti-FXa antibody or anti-FXz antibody) is bispecific, i.e., the molecule can specifically bind to two different antigens (e.g., two different epitopes on the same or different molecules). In some specific aspects, the anti-FIX antibody (e.g., anti-FIXa antibody or anti-FIXz antibody) or anti-FX antibody (e.g., anti-FXa antibody or anti-FXz antibody) is bivalent and bispecific, e.g., an antibody comprising four two-binding sites that are capable of binding to two different antigens (e.g., two different epitopes on the same or different molecules).

In some aspects, the anti-FIX antibody (e.g., anti-FIXa antibody or anti-FIXz antibody) or anti-FX antibody (e.g., anti-FXa antibody or anti-FXz antibody) disclosed herein is a human antibody, an engineered antibody, a chimeric antibody, a humanized antibody, or an optimized antibody. In some aspects the optimized antibody is an affinity optimized antibody. In some aspects, the antibody has been optimized for desirable physicochemical or functional property, e.g., extended plasma half-life, low aggregation, thermal stability, etc.

### III. Bispecific Anti-FIXa/Anti-FXz Binding Molecules

The present disclosure also provides bispecific molecules comprising an anti-FIX specificity (e.g., an anti-FIXa antibody or antigen binding portion disclosed herein or an anti-FIXz antibody or antigen binding portion thereof disclosed herein) linked to a molecule having a second binding specificity. Also provided are bispecific molecules comprising an anti-FX specificity (e.g., an anti-FXz antibody or antigen binding portion thereof disclosed herein or an anti-FXa antibody or antigen binding portion thereof disclosed herein) linked to a molecule having a second binding specificity. Also provided is a bispecific molecule comprising (i) an anti-FIX specificity (e.g., an anti-FIXa antibody or antigen binding portion disclosed herein or an anti-FIXz antibody or antigen binding portion thereof disclosed herein), linked to (ii) an anti-FXz specificity (e.g., an anti-FXz antibody or antigen binding portion thereof disclosed herein or an anti-FXa antibody or antigen binding portion thereof disclosed herein). The bispecific molecules disclosed herein are not limited to bispecific molecules with an immunoglobulin architecture or a structure derived from an antibody for example by rearranging domains. The bispecific molecules disclosed herein comprise also molecular scaffolds onto which the CDRs disclosed herein or combinations thereof can be grafted (e.g., fibronectin III or tenascin-C scaffolds).

In some aspects, the bispecific molecule cross-competes with a reference bispecific antibody, wherein the reference bispecific antibody comprises a VH and a VL of an anti-FIXa antibody selected from the group consisting of the anti-FIXa antibodies in FIG. 3A and a VH and a VL of an anti-FXz antibody selected from the group consisting of the anti-FX antibodies in FIG. 12A and FIG. 12B. In other aspects, the bispecific molecule cross-competes with a reference bispecific antibody, wherein the reference bispecific antibody comprises a VH and a VL of an anti-FIXa antibody selected from the group consisting of the anti-FIXa antibodies in FIG. 3B and a VH and a VL of an anti-FXz antibody selected from the group consisting of the anti-FXz antibodies in FIG. 12A and FIG. 12B. In some aspects, the bispecific molecule cross-competes with a reference bispecific antibody, wherein the reference bispecific antibody comprises a VH and a VL of an anti-FIXa antibody selected from the group consisting of the anti-FIXa antibodies in FIG. 3C and a VH and a VL of an anti-FXz antibody selected from the group consisting of the anti-FXz antibodies in FIG. 12A and FIG. 12B.

In certain aspects, the bispecific molecule cross-competes with a reference bispecific antibody, wherein the reference bispecific antibody comprises a VH and a VL of an anti-FIXz antibody selected from the group consisting of the anti-FIXz antibodies in FIG. 3D and a VH and a VL of an anti-FXz antibody selected from the group consisting of the anti-FXz antibodies in FIG. 12A and FIG. 12B.

Other aspects of the disclosure also provides a bispecific molecule cross-competing with a reference bispecific antibody, wherein the reference bispecific antibody comprises a VH and a VL of an anti-FIXa antibody selected from the group consisting of the anti-FIXa antibodies in FIG. 3A and a VH and a VL of an anti-FXa antibody selected from the group consisting of the anti-FXa antibodies in FIG. 12C. In some embodiments, the bispecific molecule cross-competes with a reference bispecific antibody, wherein the reference bispecific antibody comprises a VH and a VL of an anti-FIXa antibody selected from the group consisting of the anti-FIXa antibodies in FIG. 3B and a VH and a VL of an anti-FXa antibody selected from the group consisting of the anti-FXa antibodies in FIG. 12C. In other embodiments, the bispecific molecule cross-competes with a reference bispecific antibody, wherein the reference bispecific antibody comprises a VH and a VL of an anti-FIXa antibody selected from the group consisting of the anti-FIXa antibodies in FIG. 3C and a VH and a VL of an anti-FXa antibody selected from the group consisting of the anti-FXa antibodies in FIG. 12C. In yet other embodiments, the bispecific molecule cross-competes with a reference bispecific antibody, wherein the reference bispecific antibody comprises a VH and a VL of an anti-FIXz antibody selected from the group consisting of the anti-FIXz antibodies in FIG. 3D and a VH and a VL of an anti-FXa antibody selected from the group consisting of the anti-FXa antibodies in FIG. 12C.

In some aspects, the bispecific molecule binds to the same epitope as a reference bispecific antibody, wherein the reference bispecific antibody comprises a VH and a VL of an anti-FIXa antibody selected from the group consisting of the anti-FIXa antibodies in FIG. 3A and a VH and a VL of an anti-FXz antibody selected from the group consisting of the anti-FXz antibodies in FIG. 12A and FIG. 12B. In other aspects, the bispecific molecule binds to the same epitope as a reference bispecific antibody, wherein the reference bispecific antibody comprises a VH and a VL of an anti-FIXa antibody selected from the group consisting of the anti-FIXa antibodies in FIG. 3B and a VH and a VL of an anti-FXz antibody selected from the group consisting of the anti-FXz antibodies in FIG. 12A and FIG. 12B. In yet other aspects, the bispecific molecule binds to the same epitope as a reference bispecific antibody, wherein the reference bispecific antibody comprises a VH and a VL of an anti-FIXa antibody selected from the group consisting of the anti-FIXa antibodies in FIG. 3C and a VH and a VL of an anti-FXz antibody selected from the group consisting of the anti-FXz antibodies in FIG. 12A and FIG. 12B.

In some other aspects, the bispecific molecule binds to the same epitope as a reference bispecific antibody, wherein the reference bispecific antibody comprises a VH and a VL of an anti-FIXz antibody selected from the group consisting of the anti-FIXz antibodies in FIG. 3D and a VH and a VL of an anti-FXz antibody selected from the group consisting of the anti-FXz antibodies in FIG. 12A and FIG. 12B. In still other aspects, the bispecific molecule binds to the same epitope as a reference bispecific antibody, wherein the reference bispecific antibody comprises a VH and a VL of an anti-FIXz antibody selected from the group consisting of the anti-FIXz antibodies in FIG. 3D and a VH and a VL of an anti-FXa antibody selected from the group consisting of the anti-FXa antibodies in FIG. 12C.

In some aspects, the bispecific molecule binds to the same epitope as a reference bispecific antibody, wherein the reference bispecific antibody comprises a VH and a VL of an anti-FIXa antibody selected from the group consisting of the anti-FIXa antibodies in FIG. 3A and a VH and a VL of an anti-FXa antibody selected from the group consisting of the anti-FXa antibodies in FIG. 12C. In some aspects, the bispecific molecule binds to the same epitope as a reference bispecific antibody, wherein the reference bispecific antibody comprises a VH and a VL of an anti-FIXa antibody selected from the group consisting of the anti-FIXa antibodies in FIG. 3B and a VH and a VL of an anti-FXa antibody selected from the group consisting of the anti-FXa antibodies in FIG. 12C. In other aspects, In some aspects, the bispecific molecule binds to the same epitope as a reference bispecific antibody, wherein the reference bispecific antibody comprises a VH and a VL of an anti-FIXa antibody selected from the group consisting of the anti-FIXa antibodies in FIG. 3C and a VH and a VL of an anti-FXa antibody selected from the group consisting of the anti-FXa antibodies in FIG. 12C.

In some aspects, the bispecific molecule comprises

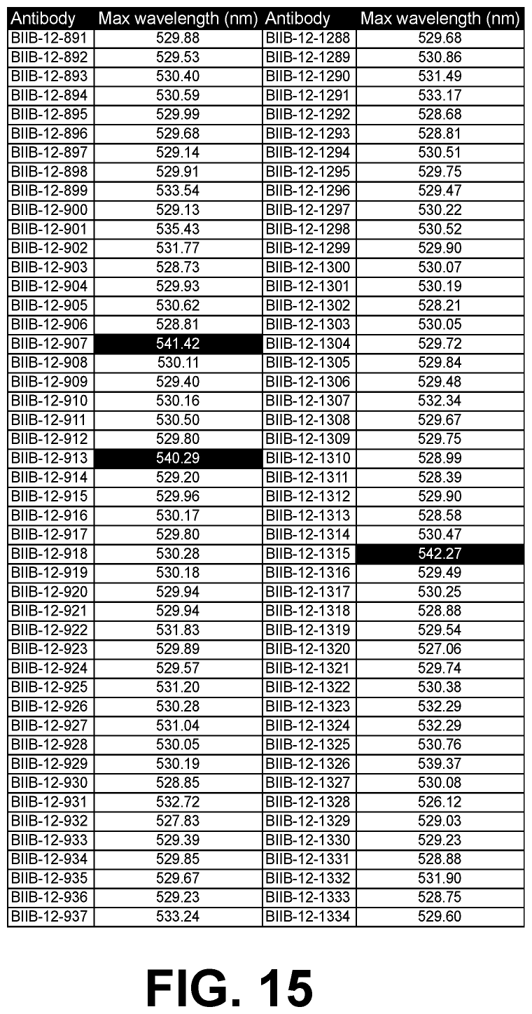

(i) an anti-FIXa antibody, or antigen binding portion thereof, comprising a VH CDR1, a VH CDR2, and a VH CDR3, and a VL CDR1, a VL CDR2, and a VL CDR3, wherein the VH CDR1, VH CDR2, and VH CDR3 and the VL CDR1, VL CDR2, and VL CDR3 are selected from the group consisting of VH CDR1s, VH CDR2s, and VH CDR3s and VL CDR1s, VL CDR2s, and VL CDR3s of the anti-FIXa (BIIB-9) antibodies in FIGS. 3A, 3B, 3C, and 3D (e.g., the CDRs of FIGS. 15A, 15B, 15C, and 15D); and
(ii) an anti-FX antibody, or antigen binding portion thereof, comprising, comprises VH CDR1, VH CDR2, and VH CDR3, and VL CDR1, VL CDR2, and VL CDR3, wherein the VH CDR1, VH CDR2, and VH CDR3 and the VL CDR1, VL CDR2, and VL CDR3 are selected from the group consisting of VH CDR1s, VH CDR2s, and VH CDR3s and VL CDR1s, VL CDR2s, and VL CDR3s of the anti-FX (BIIB-12) antibodies in FIGS. 12A and 12B (e.g., the CDRS of FIGS. 15A, 15B, 15C, and 15D).

In some aspects, the bispecific molecule comprises

(a) an anti-FIX antibody, or antigen binding portion thereof, comprising:

(a1) VH CDR1, VH CDR2, and VH CDR3 sequences comprising SEQ ID NOs: 815, 860, or 905, respectively, and/or VL CDR1, VL CDR2, and VL CDR3 sequences comprising SEQ ID NOs: 950, 995, or 1040, respectively; (BIIB-9-484)

(a2) VH CDR1, VL CDR2, and VL CDR3 sequences comprising SEQ ID NOs: 822, 867, or 912, respectively, and/or VL CDR1, VL CDR2, and VL CDR3 sequences comprising SEQ ID NOs: 957, 1002, or 1047, respectively; (BIIB-9-619)

(a3) VH CDR1, VH CDR2, and VH CDR3 sequences comprising SEQ ID NOs: 1347, 1351, or 1355, respectively, and/or VL CDR1, VL CDR2, and VL CDR3 sequences comprising SEQ ID NOs: 1359, 1363, or 1367, respectively; (BIIB-9-578)

(a4) VH CDR1, VH CDR2, and VH CDR3 sequences comprising SEQ ID NOs: 843, 888, or 933, respectively, and/or VL CDR1, VL CDR2, and VL CDR3 sequences comprising SEQ ID NOs: 978, 1023, or 1068, respectively; (BIIB-9-1335) or,

(a5) VH CDR1, VH CDR2, and VH CDR3 sequences comprising SEQ ID NOs: 844, 889, or 934, respectively, and/or VL CDR1, VL CDR2, and VL CDR3 sequences comprising SEQ ID NOs: 979, 1024, or 1069, respectively; (BIIB-9-1336) and,

(b) an anti-FX antibody, or antigen binding portion thereof, comprising:

(b1) VH CDR1, VH CDR2, and VH CDR3 sequences comprising SEQ ID NOs: 1393, 1483, or 1573, respectively, and/or VL CDR1, VL CDR2, and VL CDR3 sequences comprising SEQ ID NOs: 1663, 1753, or 1843, respectively; (BIIB-12-915)

(b2) VH CDR1, VH CDR2, and VH CDR3 sequences comprising SEQ ID NOs: 1395, 1485, or 1575, respectively, and/or VL CDR1, VL CDR2, and VL CDR3 sequences comprising SEQ ID NOs: 1665, 1755, or 1845, respectively; (BIIB-12-917)

(b3) VH CDR1, VH CDR2, and VH CDR3 sequences comprising SEQ ID NOs: 1911, 1915, or 1919, respectively, and/or VL CDR1, VL CDR2, and VL CDR3 sequences comprising SEQ ID NOs: 1923, 1927, or 1931, respectively; (BIIB-12-925)

(b4) VH CDR1, VH CDR2, and VH CDR3 sequences comprising SEQ ID NOs: 1409, 1499, or 1589, respectively, and/or VL CDR1, VL CDR2, and VL CDR3 sequences comprising SEQ ID NOs: 1679, 1769, or 1859, respectively; or (BIIB-12-932)

(b5) VH CDR1, VH CDR2, and VH CDR3 sequences comprising SEQ ID NOs: 1433, 1523, or 1613, respectively, and/or VL CDR1, VL CDR2, and VL CDR3 sequences comprising SEQ ID NOs: 1703, 1793, or 1883, respectively. (BIIB-12-1306)

In some aspects, the bispecific molecule comprises

(a) an anti-FIX antibody, or antigen binding portion thereof, comprising:

(a1) a VH and a VL comprising SEQ ID NOs: 31 and 221, respectively; (BIIB-9-484)

(a2) a VH and a VL comprising SEQ ID NOs: 45 and 235, respectively; (BIIB-9-619)

(a3) a VH and a VL comprising SEQ ID NOs: 185 and 371, respectively; (BIIB-9-578)

(a4) a VH and a VL comprising SEQ ID NOs: 87 and 221, respectively; or (BIIB-9-1335)

(a5) a VH and a VL comprising SEQ ID NOs: 89 and 221, respectively; (BIIB-9-1336)

(b) an anti-FX antibody, or antigen binding portion thereof, comprising:

(b1) a VH and a VL comprising SEQ ID NOs: 423 and 611, respectively; (BIIB-12-915)

(b2) a VH and a VL comprising SEQ ID NOs: 427 and 615, respectively; (BIIB-12-917)

(b3) a VH and a VL comprising SEQ ID NOs: 559 and 747, respectively; (BIIB-12-925)

(b4) a VH and VL comprising SEQ ID NOs: 455 and 643, respectively; or (BIIB-12-932)

(b5) a VH and a VL comprising SEQ ID NOs: 503 and 691, respectively. (BIIB-12-1306)

In some aspects, the bispecific molecule comprises an anti-FIX antibody, or antigen binding portion thereof (i.e., an anti-FIXa or an anti-FIXz antibody or antigen binding portion thereof), and an anti-FX antibody, or antigen binding portion thereof (i.e., an anti-FXz or anti-FXa antibody or antigen binding portion thereof) wherein

(i) the anti-FIXa antibody, or antigen binding portion thereof, comprises a VH and a VL comprising SEQ ID NOs: 31 and 221, respectively; (BIIB-9-484) and the anti-FXz antibody, or antigen binding portion thereof, comprises a VH and a VL comprising SEQ ID NOs: 423 and 611, respectively; (BIIB-12-915);
(ii) the anti-FIXa antibody, or antigen binding portion thereof, comprises a VH and a VL comprising SEQ ID NOs: 31 and 221, respectively; (BIIB-9-484) and the anti-FXz antibody, or antigen binding portion thereof, comprises a VH and a VL comprising SEQ ID NOs: 427 and 615, respectively; (BIIB-12-917);
(iii) the anti-FIXa antibody, or antigen binding portion thereof, comprises a VH and a VL comprising SEQ ID NOs: 31 and 221, respectively; (BIIB-9-484) and the anti-FXa antibody, or antigen binding portion thereof, comprises a VH and a VL comprising SEQ ID NOs: 559 and 747, respectively; (BIIB-12-925);
(iv) the anti-FIXa antibody, or antigen binding portion thereof, comprises a VH and a VL comprising SEQ ID NOs: 31 and 221, respectively; (BIIB-9-484) and the anti-FXz antibody, or antigen binding portion thereof, comprises a VH and a VL comprising SEQ ID NOs: 455 and 643, respectively; or (BIIB-12-932);
(v) the anti-FIXz antibody, or antigen binding portion thereof, comprises a VH and a VL comprising SEQ ID NOs: 185 and 371, respectively; (BIIB-9-578) and the anti-FXz antibody, or antigen binding portion thereof, comprises a VH and a VL comprising SEQ ID NOs: 423 and 611, respectively; (BIIB-12-915);
(vi) the anti-FIXz antibody, or antigen binding portion thereof, comprises a VH and a VL comprising SEQ ID NOs: 185 and 371, respectively; (BIIB-9-578) and the anti-FXz antibody, or antigen binding portion thereof, comprises a VH and a VL comprising SEQ ID NOs: 427 and 615, respectively; (BIIB-12-917);
(vii) the anti-FIXa antibody, or antigen binding portion thereof, comprises a VH and a VL comprising SEQ ID NOs: 45 and 235, respectively; (BIIB-9-619) and the anti-FXz antibody, or antigen binding portion thereof, comprises a VH and a VL comprising SEQ ID NOs: 427 and 615, respectively; or (BIIB-12-917)
(viii) the anti-FIXa antibody, or antigen binding portion thereof, comprises a VH and a VL comprising SEQ ID NOs: 45 and 235, respectively; (BIIB-9-619) and the anti-FXa antibody, or antigen binding portion thereof, comprises a VH and a VL comprising SEQ ID NOs: 559 and 747, respectively; (BIIB-12-925).

In some aspects, the bispecific molecule comprises an anti-FIXa antibody, or antigen binding portion thereof, comprising a VH and a VL, wherein the VH comprises an amino acid sequence at least 70%, at least 80%, at least 90%, at least 95%, at least 96%, at least 97%, at least 98%, at least 99%, or 100% identical to a VH sequence disclosed in TABLE 6 and VL comprises an amino acid sequence at least 70%, at least 80%, at least 90%, at least 95%, at least 96%, at least 97%, at least 98%, at least 99%, or 100% identical to a VL sequence disclosed in TABLE 6.

In some aspects, the bispecific molecule comprises an anti-FIXa antibody or antigen binding portion thereof, comprising a VH CDR1, a VH CDR2, and a VH CDR3, wherein the VH CDR1 sequence comprises
(iii) a VH CDR1 sequence identical to a sequence selected from the group consisting of the VH CDR1 sequences disclosed in TABLE 7, or
(iv) a VH CDR1 sequence identical to a sequence selected from the group consisting of the VH CDR1 sequences disclosed in TABLE 7 except for 1, 2, or 3 amino acid substitutions.

In some aspects, the bispecific molecule comprises an anti-FIXa antibody or antigen binding portion thereof, comprising a VH CDR1, a VH CDR2, and a VH CDR3, wherein the VH CDR2 sequence comprises
(iii) a VH CDR2 sequence identical to a sequence selected from the group consisting of the VH CDR2 sequences in TABLE 7, or
(iv) a VH CDR2 sequence identical to a sequence selected from the group consisting of the VH CDR2 sequences in TABLE 7 except for 1, 2, or 3 amino acid substitutions.

In some aspects, the bispecific molecule comprises an anti-FIXa antibody or antigen binding portion thereof, comprising a VH CDR1, a VH CDR2, and a VH CDR3, wherein the VH CDR3 sequence comprises
(iii) a VH CDR3 sequence identical to a sequence selected from the group consisting of the VH CDR3 sequences disclosed in TABLE 7, or
(iv) a VH CDR3 sequence identical to a sequence selected from the group consisting of the VH CDR3 sequences disclosed in TABLE 7 except for 1, 2, or 3 amino acid substitutions.

In some aspects, the bispecific molecule comprises an anti-FIXa antibody or antigen binding portion thereof, comprising a VL CDR1, a VL CDR2, and a VL CDR3, wherein the VL CDR1 sequence comprises
(v) a VL CDR1 sequence identical to a sequence selected from the group consisting of the VL CDR1 sequences disclosed in TABLE 7, or
(vi) a VL CDR1 sequence identical to a sequence selected from the group consisting of the VL CDR1 sequences disclosed in TABLE 7 except for 1, 2, or 3 amino acid substitutions.

In some aspects, the bispecific molecule comprises an anti-FIXa antibody or antigen binding portion thereof, comprising a VL CDR1, a VL CDR2, and a VL CDR3, wherein the VL CDR2 sequence comprises
(v) a VL CDR2 sequence identical to a sequence selected from the group consisting of the VL CDR2 sequences in TABLE 7, or
(vi) a VL CDR2 sequence identical to a sequence selected from the group consisting of the VL CDR2 sequences in TABLE 7 except for 1, 2, or 3 amino acid substitutions.

In some aspects, the bispecific molecule comprises an anti-FIXa antibody or antigen binding portion thereof, comprising a VL CDR1, a VL CDR2, and a VL CDR3, wherein the VL CDR3 sequence comprises
(v) a VL CDR3 sequence identical to a sequence selected from the group consisting of the VL CDR3 sequences disclosed in TABLE 7, or
(vi) a VL CDR3 sequence identical to a sequence selected from the group consisting of the VL CDR3 sequences disclosed in TABLE 7 except for 1, 2, or 3 amino acid substitutions.

In some aspects, the anti-FIXa antibody or antigen binding portion thereof, comprises a VH CDR1, a VH CDR2, and a VH CDR3, and VL CDR1, a VL CDR2, and a VL CDR3, selected from the VH CDR1, a VH CDR2, and a VH CDR3, and VL CDR1, a VL CDR2, and a VL CDR3 sequences disclosed in TABLE 7.

Some aspects of the disclosure pertains to a bispecific molecule that specifically binds to FIX and FX and then functionally mimics activated factor VIII (FVIIIa) cofactor in at least one FVIIIa activity assay. In some aspects, the FVIIIa activity assay is selected from a chromogenic FXa generation assay, a one-stage clotting assay, or the combination thereof. A particular aspect of the disclosure includes a bispecific molecule that preferentially binds to FIXa (e.g, FIXa in tenase complex, e.g., FIXa-SM) over free FIXa or FIX zymogen and FX zymogen over FXa (e.g., FXa-SM) and mimics an activated factor VIII cofactor activity.

In some embodiments, the FVIIIa activity achieves at least about 10%, at least about 15%, at least about 20%, at least about 25%, at least about 30%, at least about 35%, at least about 40%, at least about 45%, at least about 50%, at least about 55%, at least about 60%, at least about 60%, at least about 65%, at least about 70%, at least about 75%, at least about 80%, at least about 85%, at least about 90%, at least about 95%, at least about 100%, at least about 105%, at least about 110%, at least about 115%, at least about 120%, at least about 125%, at least about 130%, at least about 135%, at least about 140%, at least about 145%, at least about 150%, at least about 155%, at least about 160%, at least about 165%, at least about 170%, at least about 175%, at least about 180%, at least about 185%, at least about 190%, at least about 195%, or at least about 200% of the activity otherwise achieved by FVIII in the same assay.

In further embodiments, the bispecific molecule is capable of generating thrombin from prothrombin, fibrin from fibrinogen, and/or fibrin clot in vitro or in vivo. In some embodiments, the bispecific molecule concurrently binds to both FIXa and FX, as determined by BLI.

In some aspects, the bispecific molecule comprises an anti-FIX antibody, or antigen binding portion thereof (i.e., an anti-FIXa or an anti-FIXz antibody or antigen binding portion thereof), and an anti-FX antibody, or antigen binding portion thereof (i.e., an anti-FXz or anti-FXa antibody or antigen binding portion thereof), wherein the bispecific molecule shows significant loss of activity in the absence of phospholipids as measured in an FXa generation assay. In some aspects, the bispecific molecule comprises an anti-FIXa antibody or antigen binding protein thereof (i.e., an anti-FIXa or an anti-FIXz antibody or antigen binding portion thereof) and an anti-FX antibody, or antigen binding portion thereof (i.e., an anti-FXz or anti-FXa antibody or antigen binding portion thereof). In some aspects, the absence of phospholipids in the FXa generation assay results in a loss of activity of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%, with respect to the activity measured in the presence of phospholipids. In some aspects, the bispecific molecule shows minimal phospholipid-independent activity compared to the phospholipid-independent activity of a reference bispecific antibody disclosed in U.S. Pat. No. 8,062,635 (e.g., emicizumab, ACE910), which is incorporated herein by reference in its entirety. In some aspects, the bispecific molecule disclosed herein shows less than about 20%, less than about 15%, less than about 10%, or less than about 5% of the activity observed for a reference bispecific antibody disclosed in U.S. Pat. No. 8,062,635 (e.g., emicizumab, ACE910) in an FXa generation assay in the absence of phospholipids.

In some aspect, the bispecific molecule disclosed herein has higher activity in a thrombin generation assay triggered with factor XIa when the synthetic phospholipid vesicles tested are composed of PS (phosphatidylserine)/PE (phosphatidylethanolamine)/PC(phosphatidylcholine) (20%/40%/40%) than when the synthetic phospholipid vesicles tested are composed of PS/PC (20%/80%). In some aspects, the activity of the bispecific molecule disclosed herein in a thrombin generation assay triggered with factor XIa in the presence of PE-containing phospholipid vesicles (e.g, PS/PE/PC 20%/40%/40%) is at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300% higher than the activity observed under the same experimental conditions in the presence of vesicles lacking PE (e.g., PS/PC 20%/80%).

In some aspects, the phospholipid concentration that supports peak activity for the bispecific molecule of the present disclosure is higher than the phospholipid concentration that supports peak activity for rFVIII.

In some aspects, the bispecific molecule is of the IgG isotype. In some aspects, the IgG isotype is of the IgG1 subclass. In some aspects, the IgG isotype is of the IgG4 subclass.

In some aspects the bispecific molecule is of a bispecific IgG format and is selected from the group consisting of the bispecific antibodies in TABLE 2. In some aspects, the bispecific molecule is of a bispecific heterodimeric format.

In some aspects, the bispecific molecule comprises two different heavy chains and two different light chains. In some aspects, the bispecific molecule comprises two identical light chains and two different heavy chains.

In some aspects, the bispecific molecule is capable of controlling or reducing the incidence of bleeding episodes in a subject having hemophilia. In some aspects, the bispecific molecule is capable of maintaining homeostasis or in a subject having hemophilia.

In some aspects, the bispecific molecule is capable of providing routine prophylaxis in a subject having hemophilia. In some aspects, the subject has developed or is expected to develop neutralizing antibodies against Factor VIII.

In some aspects, the bispecific molecule disclosed herein (e.g., an antibody) is a (monoclonal) bispecific antibody that has binding specificities for at least two different sites and can be of any format. A wide variety of recombinant antibody formats have been developed in the recent past, e.g. bivalent, trivalent or tetravalent bispecific antibodies. Examples include the fusion of an IgG antibody format and single chain domains (for different formats see e.g. Coloma, M. J., et al, Nature Biotech 15 (1997), 159-163; WO 2001/077342; Morrison, S. L., Nature Biotech 25 (2007), 1233-1234; Holliger. P. et. al, Nature Biotech. 23 (2005), 1 126-1 136; Fischer, N., and Leger, O., Pathobiology 74 (2007), 3-14; Shen, J., et. al, J. Immunol. Methods 318 (2007), 65-74; Wu, C, et al., Nature Biotech. 25 (2007), 1290-1297).

The bispecific antibody or fragment herein also includes bivalent, trivalent or tetravalent bispecific antibodies produced according to the methods disclosed in WO2009/080251; WO2009/080252; WO 2009/080253; WO2009/080254; WO2010/112193; WO2010/115589; WO2010/136172; WO2010/145792; WO2010/145793 and WO2011/117330, all of which are herein incorporated by reference in their entireties.

In some aspects, the bispecific molecule, e.g., an antibody, disclosed herein comprises an Fd, scFv, disulfide stabilized scFv, a disulfide linked Fv, a V-NAR domain, an IgNar, an intrabody, an IgG-CH2, a minibody, a F(ab′)₃, a tetrabody, a triabody, a diabody, a single-domain antibody, DVD-Ig, Fcab, mAb², a (scFv)₂, or a scFv-Fc.

The bispecific antibodies disclosed herein may be bispecific even in cases where there are more than two binding domains (i.e., the antibody is trivalent or multivalent). Bispecific antibodies include, for example, multivalent single chain antibodies, diabodies and triabodies, as well as antibodies having the constant domain structure of full length antibodies to which further antigen-binding domains (e.g., single chain Fv, a VH domain and/or a VL domain, Fab, or (Fab)2) are linked via one or more peptide-linkers. The antibodies can be full length from a single species, or be chimerized or humanized. For an antibody with more than two antigen binding domains, some binding domains may be identical, as long as the protein has binding domains for two different antigens.

The term “valent” as used within the current application denotes the presence of a specified number of binding domains in an antibody molecule. As such, the terms “bivalent”, “tetravalent”, and “hexavalent” denote the presence of two binding domains, four binding domains, and six binding domains, respectively, in an antibody molecule. The bispecific antibodies disclosed herein are at least “bivalent” and may be “trivalent” or “multivalent” (e.g. “tetravalent” or “hexavalent”). In some aspects, the bispecific antibody according to the invention is bivalent, trivalent or tetravalent.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al, EMBO J. 10: 3655 (1991)), and “knob-in-hole” engineering (see, e.g., U.S. Pat. No. 5,731,168; U.S. Publication No. 2011/0287009). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al, Science, 229: 81 (1985)); using leucine zippers or coiled coils to produce bispecific antibodies (see, e.g., Kostelny et al, J. Immunol, 148(5): 1547-1553 (1992) and WO2011/034605); using a furin cleavable tether between a CL domain and a VH domain in a single VH/VL unit (see, e.g., WO2013/119966 and WO2013/055958); using “diabody” technology for making bispecific antibody fragments (see, e.g., Hollinger et al., Proc. Natl. Acad. Sci. USA, 90:6444-6448 (1993)); using immunoglobulin domain crossover for making bispecific antibodies (see, e.g., WO2009/080251); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Bispecific binding molecules in the context of the present disclosure may relate to an antibody molecule comprising two antibody-derived binding domains, wherein one binding domain can be a scFv. One of the binding domains consists of variable regions (or parts thereof) of an antibody, antibody fragment or derivate thereof, capable of specifically binding to/interacting with a first target molecule (e.g., FIXa). The second binding domain consists of variable regions (or parts thereof) of an antibody, antibody fragment or derivative thereof, capable of specifically binding to/interacting with a second target molecule (e.g., FXz).

The two domains/regions in the bispecific antibody molecule are preferably covalently connected to one another. This connection can be effected directly, e.g., domain 1 [specific for a first (human) target molecule, e.g., FIXa]-domain 2 [specific for second (human) target molecule, e.g., FXz] or vice versa. In other aspects, this connection can be effected through an additional polypeptide linker sequence [domain 1]-[linker sequence]-[domain2].

In the event that a linker is used, this linker is in the context of the present disclosure of a length and sequence sufficient to ensure that each of the first and second domains can, independently from each other, retain their differential binding specificities. In the context of the present disclosure the additional polypeptide linker sequence can also be a fragment of an antibody itself which may be for example the Fc part or one or more constant domains of an antibody.

In the context of the present disclosure, binding domain 1 can also be part of an antibody arm 1 and binding domain 2 can also be part of an antibody arm 2, or vice versa, wherein the two antibody arms are connected via an interface. The antibody arm 1 consists of variable regions (or parts thereof) of an antibody, antibody fragment or derivate thereof, capable of specifically binding to/interacting with a (human) target molecule 1. The antibody arm 2 consists of variable regions (or parts thereof) of an antibody, antibody fragment or derivative thereof capable of specifically binding to/interacting with a (human) target molecule 2.

The “interface” comprises those contact amino acid residues (or other non-amino acid groups such as, e.g., carbohydrate groups) in the first antibody arm which interact with one or more “contact” amino acid residues (or other non-amino acid groups) in the interface of the second antibody arm. The preferred interface is a domain of an immunoglobulin such as a constant domain (or regions thereof) of the antibody's heavy chains, wherein the binding/interaction via the interface provides for the heterodimerization of the two antibody arms. See e.g. Ridgway et al. (1996) Protein Eng. 9:617-621; International Publ. No. WO 96/027011; Merchant et al. (1998) Nature Biotech. 16:677-681; Atwell et al. (1997) J. Mol. Biol. 270:26-35; European Patent Application EP1870459A1; and International Publication Nos. WO2007/147901, WO2009/089004 and WO 2010/129304, all of which are herein incorporated by reference in their entireties.

Bispecific antibody molecule to be employed in accordance with the disclosure can be further modified using conventional techniques known in the art, for example, by using amino acid deletion(s), insertion(s), substitution(s), addition(s), and/or recombination(s) and/or any other modification(s) known in the art either alone or in combination. Methods for introducing such modifications in the DNA sequence underlying the amino acid sequence of an immunoglobulin chain arc well known to the person skilled in the art; see, e.g., Sambrook (1989), loc. cit. Fragments or derivatives of the recited Ig-derived domains define (poly) peptides which are parts of the above antibody molecules and/or which are modified by chemical/biochemical or molecular biological methods. Corresponding methods are known in the art and described inter alia in laboratory manuals (see Sambrook et al., Molecular Cloning: A Laboratory Manual: Cold Spring Harbor Laboratory Press, 2nd edition (1989) and 3rd edition (2001); Gerhardt et al., Methods for General and Molecular Bacteriology ASM Press (1994); Lefkovits, Immunology Methods Manual: The Comprehensive Sourcebook of Techniques; Academic Press (1997); Golemis, Protein-Protein Interactions: A Molecular Cloning Manual Cold Spring Harbor Laboratory Press (2002)).

The bispecific antibodies disclosed herein can comprise, for example, one or more of the following components:

(i) “Single-chain Fvs” or “scFv”: Antibody fragments that have the VH and VL domains of an antibody, wherein these domains are present in a single polypeptide chain. Generally, the scFv polypeptide further comprises a polypeptide linker between the VH and VL domains which enables the scFv to form the desired structure for antigen binding. Techniques described for the production of single chain antibodies are described, e.g., in Pluckhun in The Pharmacology of Monoclonal Antibodies, Rosenburg and Moore eds. Springer-Verlag, N.Y. 113 (1994), 269-315.
(ii) “Fab fragment”: Comprised of one light chain and the CH1 and variable regions of one heavy chain. The heavy chain of a Fab molecule cannot form a disulfide bond with another heavy chain molecule.
(iii) “Fab′ fragment”: Contains one light chain and a portion of one heavy chain that contains the VH domain and the CH1 domain and also the region between the CH1 and CH2 domains, such that an interchain disulfide bond can be formed between the two heavy chains of two Fab′ fragments to form a F(ab′)₂ molecule.
(iv) “F(ab′)₂ fragment”: Contains two light chains and two heavy chains containing a portion of the constant region between the CH1 and CH2 domains, such that an interchain disulfide bond is formed between the two heavy chains. A F(ab′)₂ fragment thus is composed of two Fab′ fragments that are held together by a disulfide bond between the two heavy chains. The “Fv region” comprises the variable regions from both the heavy and light chains, but lacks the constant regions.

It is of note that a bispecific antibody disclosed herein may comprise, in addition to the herein defined first (Ig-derived) domain and the (Ig-derived) second domain (an) additional domain(s), e.g. for the isolation and/or preparation of recombinantly produced constructs.

### IV. Constant Region of Antibodies

An anti-FIX variable region (e.g., anti-FIXa variable region or anti-FIXz variable region) and/or anti-FX variable region (e.g., anti-FXa variable region or anti-FXz variable region) described herein, as monospecific, bispecific, or multispecific molecules, can be linked (e.g., covalently linked or fused) to an Fc, e.g., an IgG1, IgG2, IgG3 or IgG4 Fc, which may be of any allotype or isoallotype, e.g., for IgG1: G1m, G1m1(a), G1m2(x), G1m3(f), G1m17(z); for IgG2: G2m, G2m23(n); for IgG3: G3m, G3m21(g1), G3m28(g5), G3m11(b0), G3m5(b1), G3m13(b3), G3m14(b4), G3m10(b5), G3m15(s), G3m16(t), G3m6(c3), G3m24(c5), G3m26(u), G3m27(v); and for K: Km, Km1, Km2, Km3 (see, e.g., Jefferies et al. (2009) mAbs 1: 1).

In certain embodiments, anti-FIX or anti-FX variable regions described herein are linked to an Fc that binds to one or more activating Fc receptors (FcγI, FcγIIa or FcγIIIa), and thereby stimulate ADCC. In certain embodiments, anti-FIX or anti-FX variable regions described herein are linked to an effectorless or mostly effectorless Fc, e.g., IgG2 or IgG4.

Anti-FIX or anti-FX variable regions described herein can be linked to a non-naturally occurring Fc region, e.g., an effectorless Fc or an Fc with enhanced binding to one or more activating Fc receptors (FcγI, FcγIIa or FcγIIIa).

Generally, variable regions described herein can be linked to an Fc comprising one or more modification, typically to alter one or more functional properties of the antibody, such as serum half-life, complement fixation, Fc receptor binding, and/or antigen-dependent cellular cytotoxicity. Furthermore, an antibody described herein can be chemically modified (e.g., one or more chemical moieties can be attached to the antibody) or be modified to alter its glycosylation, to alter one or more functional properties of the antibody. Each of these embodiments is described in further detail below. The numbering of residues in the Fc region is that of the EU index of Kabat.

The Fc region encompasses domains derived from the constant region of an immunoglobulin, preferably a human immunoglobulin, including a fragment, analog, variant, mutant or derivative of the constant region. Suitable immunoglobulins include IgG1, IgG2, IgG3, IgG4, and other classes such as IgA, IgD, IgE and IgM. The constant region of an immunoglobulin is defined as a naturally-occurring or synthetically-produced polypeptide homologous to the immunoglobulin C-terminal region, and can include a CH1 domain, a hinge, a CH2 domain, a CH3 domain, or a CH4 domain, separately or in combination.

The constant region of an immunoglobulin is responsible for many important antibody functions including Fc receptor (FcR) binding and complement fixation. There are five major classes of heavy chain constant region, classified as IgA, IgG, IgD, IgE, IgM, each with characteristic effector functions designated by isotype. For example, IgG is separated into four subclasses known as IgG1, IgG2, IgG3, and IgG4. Ig molecules interact with multiple classes of cellular receptors. For example IgG molecules interact with three classes of Fcγ receptors (FcγR) specific for the IgG class of antibody, namely FcγRI, FcγRII, and FcγRIII The important sequences for the binding of IgG to the FcγR receptors have been reported to be located in the CH2 and CH3 domains. The serum half-life of an antibody is influenced by the ability of that antibody to bind to an Fc receptor (FcR).

In certain embodiments, the Fc region is a variant Fc region, e.g., an Fc sequence that has been modified (e.g., by amino acid substitution, deletion and/or insertion) relative to a parent Fc sequence (e.g., an unmodified Fc polypeptide that is subsequently modified to generate a variant), to provide desirable structural features and/or biological activity,

For example, one may make modifications in the Fc region in order to generate an Fc variant that (a) has increased or decreased antibody-dependent cell-mediated cytotoxicity (ADCC), (b) increased or decreased complement mediated cytotoxicity (CDC), (c) has increased or decreased affinity for C1q and/or (d) has increased or decreased affinity for a Fc receptor relative to the parent Fc. Such Fc region variants will generally comprise at least one amino acid modification in the Fc region. Combining amino acid modifications is thought to be particularly desirable. For example, the variant Fc region may include two, three, four, five, etc substitutions therein, e.g., of the specific Fc region positions identified herein.

A variant Fc region may also comprise a sequence alteration wherein amino acids involved in disulfide bond formation are removed or replaced with other amino acids. Such removal may avoid reaction with other cysteine-containing proteins present in the host cell used to produce the antibodies described herein. Even when cysteine residues are removed, single chain Fc domains can still form a dimeric Fc domain that is held together non-covalently. In other embodiments, the Fc region may be modified to make it more compatible with a selected host cell. For example, one may remove the PA sequence near the N-terminus of a typical native Fc region, which may be recognized by a digestive enzyme in E. coli such as proline iminopeptidase. In other embodiments, one or more glycosylation sites within the Fc domain may be removed. Residues that are typically glycosylated (e.g., asparagine) may confer cytolytic response. Such residues may be deleted or substituted with unglycosylated residues (e.g., alanine). In other embodiments, sites involved in interaction with complement, such as the C1q binding site, may be removed from the Fc region. For example, one may delete or substitute the EKK sequence of human IgG1. In certain embodiments, sites that affect binding to Fc receptors may be removed, preferably sites other than salvage receptor binding sites. In other embodiments, an Fc region may be modified to remove an ADCC site. ADCC sites are known in the art; see, for example, Molec. Immunol. 29 (5): 633-9 (1992) with regard to ADCC sites in IgG1. Specific examples of variant Fc domains are disclosed for example, in WO 97/34631 and WO 96/32478.

In one embodiment, the hinge region of Fc is modified such that the number of cysteine residues in the hinge region is altered, e.g., increased or decreased. This approach is described further in U.S. Pat. No. 5,677,425 by Bodmer et al. The number of cysteine residues in the hinge region of Fc is altered to, for example, facilitate assembly of the light and heavy chains or to increase or decrease the stability of the antibody. In one embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcal protein A (SpA) binding relative to native Fc-hinge domain SpA binding. This approach is described in further detail in U.S. Pat. No. 6,165,745 by Ward et al.

In yet other embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the CI component of complement. This approach is described in further detail in U.S. Pat. Nos. 5,624,821 and 5,648,260, both by Winter et al.

In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In yet another example, the Fc region may be modified to increase antibody dependent cellular cytotoxicity (ADCC) and/or to increase the affinity for an Fcγ receptor by modifying one or more amino acids at the following positions: 234, 235, 236, 238, 239, 240, 241, 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 312, 313, 315, 320, 322, 324, 325, 326, 327, 329, 330, 331, 332, 333, 334, 335, 337, 338, 340, 360, 373, 376, 378, 382, 388, 389, 398, 414, 416, 419, 430, 433, 434, 435, 436, 437, 438 or 439. Exemplary substitutions include 236A, 239D, 239E, 268D, 267E, 268E, 268F, 324T, 332D, and 332E. Exemplary variants include 239D/332E, 236A/332E, 236A/239D/332E, 268F/324T, 267E/268F, 267E/324T, and 267E/268F7324T. Other modifications for enhancing FcγR and complement interactions include but are not. limited to substitutions 298 A, 333A, 334A, 326A, 2471, 339D, 339Q, 280H, 290S, 298D, 298V, 243L, 292P, 300L, 396L, 3051, and 396L. These and other modifications are reviewed in Strohl, 2009, Current Opinion in Biotechnology 20:685-691.

Fc modifications that increase binding to an Fcγ receptor include amino acid modifications at any one or more of amino acid positions 238, 239, 248, 249, 252, 254, 255, 256, 258, 265, 267, 268, 269, 270, 272, 279, 280, 283, 285, 298, 289, 290, 292, 293, 294, 295, 296, 298, 301, 303, 305, 307, 312, 315, 324, 327, 329, 330, 335, 337, 3338, 340, 360, 373, 376, 379, 382, 388, 389, 398, 414, 416, 419, 430, 434, 435, 437, 438 or 439 of the Fc region, wherein the numbering of the residues in the Fc region is that of the EU index as in Kabat (WO00/42072).

Other Fc modifications that can be made to Fes are those for reducing or ablating binding to FcγR and/or complement proteins, thereby reducing or ablating Fc-mediated effector functions such as ADCC, ADCP, and CDC. Exemplary modifications include but are not limited substitutions, insertions, and deletions at positions 234, 235, 236, 237, 267, 269, 325, and 328, wherein numbering is according to the EU index. Exemplary substitutions include but are not limited to 234G, 235G, 236R, 237K, 267R, 269R, 325L, and 328R, wherein numbering is according to the EU index. An Fc variant may comprise 236R/328R. Other modifications for reducing FcγR and complement interactions include substitutions 297A, 234A, 235A, 237A, 318A, 228P, 236E, 268Q, 309L, 330S, 331 S, 220S, 226S, 229S, 238S, 233P, and 234V, as well as removal of the glycosylation at position 297 by mutational or enzymatic means or by production in organisms such as bacteria that do not glycosylate proteins. These and other modifications are reviewed in Strohl, 2009, Current Opinion in Biotechnology 20:685-691.

Optionally, the Fc region may comprise a non-naturally occurring amino acid residue at additional and/or alternative positions known to one skilled in the art (see, e.g., U.S. Pat. Nos. 5,624,821; 6,277,375; 6,737,056; 6,194,551; 7,317,091; 8,101,720; PCX Patent Publications WO 00/42072; WO 01/58957; WO 02/06919; WO 04/016750; WO 04/029207; WO 04/035752; WO 04/074455; WO 04/099249; WO 04/063351; WO 05/070963; WO 05/040217, WO 05/092925 and WO 06/020114).

Fc variants that enhance affinity for an inhibitory receptor FcγRIIb may also be used. Such variants may provide an Fc fusion protein with immuno-modulatory activities related to FcγRIIb⁺ cells, including for example B cells and monocytes. In one embodiment, the Fc variants provide selectively enhanced affinity to FcγRIIb relative to one or more activating receptors. Modifications for altering binding to FcγRIIb include one or more modifications at a position selected from the group consisting of 234, 235, 236, 237, 239, 266, 267, 268, 325, 326, 327, 328, and 332, according to the EU index. Exemplary substitutions for enhancing FcγRIIb affinity include but are not limited to 234D, 234E, 234F, 234W, 235D, 235F, 235R, 235Y, 236D, 236N, 237D, 237N, 239D, 239E, 266M, 267D, 267E, 268D, 268E, 327D, 327E, 328F, 328W, 328Y, and 332E. Exemplary substitutions include 235Y, 236D, 239D, 266M, 267E, 268D, 268E, 328F, 328W, and 328Y. Other Fc variants for enhancing binding to FcγRIIb include 235Y/267E, 236D/267E, 239D/268D, 239D/267E, 267E/268D, 267E/268E, and 267E/328F.

The affinities and binding properties of an Fc region for its ligand may be determined by a variety of in vitro assay methods (biochemical or immunological based assays) known in the art including but not limited to, equilibrium methods (e.g., enzyme-linked immunoabsorbent assay (ELISA), or radioimmunoassay (RIA)), or kinetics (e.g., BIACORE analysis), and other methods such as indirect binding assays, competitive inhibition assays, fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental immunology, 4th Ed., Lippincott-Raven, Philadelphia (1999), which focuses on antibody-immunogen interactions.

In certain embodiments, the antibody is modified to increase its biological half-life. Various approaches are possible. For example, this may be done by increasing the binding affinity of the Fc region for FcRn, For example, one or more of more of following residues can be mutated: 252, 254, 256, 433, 435, 436, as described in U.S. Pat. No. 6,277,375. Specific exemplary substitutions include one or more of the following: T252L, T254S, and/or T256F. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. Other exemplary variants that increase binding to FcRn and/or improve pharmacokinetic properties include substitutions at positions 259, 308, 428, and 434, including for example 2591, 308F, 428L, 428M, 434S, 4341 1. 434F, 434Y, and 434X1. Other variants that increase Fc binding to FcRn include: 250E, 250Q, 428 L, 428F, 250Q/428L (Hinton et al, 2004, J. Biol. Chem. 279(8): 6213-6216, Hinton et al. 2006 Journal of Immunology 176:346-356), 256A, 272A, 286A, 305A, 307A, 307Q, 31 IA, 312A, 376A, 378Q, 380A, 382A, 434A (Shields et al, Journal of Biological Chemistry, 2001, 276(9):6591-6604), 252F, 252T, 252Y, 252W, 254T, 256S, 256R, 256Q, 256E, 256D, 256T, 309P, 31 1 S, 433R, 433S, 4331, 433P, 433Q, 434H, 434F, 434Y, 252Y/254T/256E, 433K/434F/436H, 308T/309P/31IS (Dall'Acqua et al. Journal of Immunology, 2002, 169:5171-5180, Dall'Acqua et al., 2006, Journal of Biological Chemistry 281:23514-23524). Other modifications for modulating FcRn binding are described in Yeung et al., 2010, J Immunol, 182:7663-7671. In certain embodiments, hybrid IgG isotypes with particular biological characteristics may be used. For example, an IgG1/IgG3 hybrid variant may be constructed by substituting IgG ! positions in the CH2 and/or CH3 region with the amino acids from IgG3 at positions where the two isotypes differ. Thus a hybrid variant IgG antibody may be constructed that comprises one or more substitutions, e.g., 274Q, 276K, 300F, 339T, 356E, 358M, 384S, 392N, 397M, 4221, 435R, and 436F. In other embodiments described herein, an IgG1/IgG2 hybrid variant may be constructed by substituting IgG2 positions in the CH2 and/or CH3 region with amino acids from IgG1 at positions where the two isotypes differ. Tus a hybrid variant IgG antibody may be constructed chat comprises one or more substitutions, e.g., one or more of the following amino acid substitutions: 233E, 234L, 235L, -236G (referring to an insertion of a glycine at position 236), and 321 h.

Moreover, the binding sites on human IgG1 for FcγRI, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 were shown to improve binding to FcγRIII. Additionally, the following combination mutants were shown to improve FcγRIII binding: T256A/S298A, S298A/E333A,

S298A/K224A and S298A/E333A/K334A, which has been shown to exhibit enhanced FcγRIIIa binding and ADCC activity (Shields et al., 2001). Other IgG1 variants with strongly enhanced binding to FcγRIIIa have been identified, including variants with S239D/I332E and

S239D/I332E/A330L mutations which showed the greatest increase in affinity for FcγRIIIa, a decrease in FcγRIIb binding, and strong cytotoxic activity in cynomolgus monkeys (Lazar et al., 2006). Introduction of the triple mutations into antibodies such as alemtuzumab (CD52-specific), trastuzumab (HER2/neu-specific), rituximab (CD20-specific), and cetuximab (EGFR-specific) translated into greatly enhanced ADCC activity in vitro, and the S239D/I332E variant showed an enhanced capacity to deplete B cells in monkeys (Lazar et al., 2006). In addition, IgG1 mutants containing L235V, F243L, R292P, Y300L and P396L mutations which exhibited enhanced binding to FcγRIIIa and concomitantly enhanced ADCC activity in transgenic mice expressing human FcγRIIIa in models of B cell malignancies and breast cancer have been identified (Stavenhagen et al., 2007; Nordstrom et al., 2011). Other Fc mutants that may be used include: S298A/E333A/L334A, S239D/I332E, S239D/I332E/A330L, L235V/F243L/R292P/Y300L/P396L, and M428L/N434S.

In certain embodiments, an Fc is chosen that has reduced binding to FcγRs. An exemplary Fc, e.g., IgG1 Fc, with reduced FcγR binding comprises the following three amino acid substitutions: L234A, L235E and G237A.

In certain embodiments, an Fc is chosen that has reduced complement fixation. An exemplary Fc, e.g., IgG1 Fc, with reduced complement fixation has the following two amino acid substitutions: A330S and P331S.

In certain embodiments, an Fc is chosen that has essentially no effector function, i.e., it has reduced binding to FcγRs and reduced complement fixation. An exemplary Fc, e.g., IgG1 Fc, that is effectorless comprises the following five mutations: L234A, L235E, G237A, A330S and P331S.

When using an IgG4 constant domain, it is usually preferable to include the substitution S228P, which mimics the hinge sequence in IgG1 and thereby stabilizes IgG4 molecules.

It will be noted that in certain aspects, the binding molecules disclosed herein can be engineered to fuse the CH3 domain directly to the hinge region of the respective modified antibodies or fragments thereof. In other constructs, a peptide spacer can be inserted between the hinge region and the modified CH2 and/or CH3 domains. For example, compatible constructs can be expressed wherein the CH2 domain has been deleted and the remaining CH3 domain (modified or unmodified) is joined to the hinge region with a 5-20 amino acid spacer. Such a spacer can be added, for instance, to ensure that the regulatory elements of the constant domain remain free and accessible or that the hinge region remains flexible. However, it should be noted that amino acid spacers can, in some cases, prove to be immunogenic and elicit an unwanted immune response against the construct. Accordingly, in certain aspects, any spacer added to the construct will be relatively non-immunogenic, or even omitted altogether, so as to maintain the desired biochemical qualities of the modified antibodies.

Besides the deletion of whole constant region domains, it will be appreciated that the binding molecules disclosed herein can be provided by the partial deletion or substitution of a few or even a single amino acid. For example, the mutation of a single amino acid in selected areas of the CH2 domain can be enough to substantially reduce Fc binding and thereby increase tumor localization. Moreover, as alluded to above, the constant regions of the disclosed binding molecules can be modified through the mutation or substitution of one or more amino acids that enhances the profile of the resulting construct. In this respect it is possible to disrupt the activity provided by a conserved binding site (e.g., Fc binding) while substantially maintaining the configuration and immunogenic profile of the modified antibody or antigen-binding fragment thereof. Certain aspects can comprise the addition of one or more amino acids to the constant region to enhance desirable characteristics such as decreasing effector function or provide for more therapeutic or diagnostic agent attachment. In such aspects, specific sequences derived from selected constant region domains can be inserted or replicated.

In still another embodiment, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Glycosylation of the constant region on N297 can be prevented by mutating one or more of the amino acids residues (e.g., the glycoslylated amino acid residues or an adjacent amino acids residue) to another residue, e.g., N297A, S298G, T299A, or any combination thereof.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies described herein to thereby produce an antibody with altered glycosylation. For example, EP 1,176,195 by Hanai et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Led 3 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17: 176-180).

Another modification of the antibodies described herein is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (CI-CIO) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies described herein. See for example, EP 0 154 316 by Nishimura et al. and EP 0 401 384 by Ishikawa et al.

### V. Immunoconjugates and Fusion Proteins

The present disclosure also provides immunoconjugates comprising any of the binding molecules (e.g., antibodies) disclosed herein (e.g., an anti-FIX, anti-FX, or a bispecific anti-FIX/anti-FX antibody disclosed herein). In one aspect, the immunoconjugate comprises an antibody or antigen binding portion disclosed herein linked to an agent. In one particular aspect, the immunoconjugate comprises a bispecific molecule disclosed herein linked to an agent (e.g., as therapeutic agent or a diagnostic agent).

Accordingly, the present disclosure provides immunoconjugates based on the anti-FIX disclosed herein, based on the anti-FX disclosed herein, or based on the bispecific antibodies disclosed herein, e.g., bispecific antibodies comprising an anti-FIX specificity and an anti-FX specificity.

In some aspects, the immunoconjugate comprises any of the binding molecules disclosed herein (e.g., an anti-FIX, anti-FX, or a bispecific anti-FIX/anti-FX antibody disclosed herein) conjugated to at least one therapeutic or diagnostic agent. In some aspects, the immunoconjugate further comprises at least one optional spacer which can be intercalated between the side chain or an amino acid in a polypeptide chain of a binding molecule disclosed herein (e.g., an anti-FIX, anti-FX, or a bispecific anti-FIX/anti-FX antibody disclosed herein), and the therapeutic or diagnostic moiety. In some aspects, the at least one spacer is a peptide spacer, In other aspects, the at one spacer is a non-peptidic spacer. In some aspects, the spacer is unstable, such as an acid labile spacer (e.g., a hydrazine). In other aspects, the spacer is an enzyme cleavable peptide, e.g., a cleavable dipeptide. In some aspects, the spacer is uncleavable (hydrolytically stable), for example, a thioether spacer or a hindered disulfide spacer.

In some aspects, the immunoconjugate comprises two, three, four, five, six, seven, eight, nine or ten therapeutic or diagnostic moieties. In some aspects, all therapeutic or diagnostic moieties are the same. In some aspects, at least one therapeutic or diagnostic moiety is different from the rest. In some aspects, all therapeutic or diagnostic moieties are different. In some aspects, all the spacers (e.g., peptidic and/or non-peptidic spacers) are the same. In some aspects, at least one spacer is different from the rest. In still other aspects, all the spacers are different.

In some aspects, each therapeutic or diagnostic moiety is chemically conjugated to the side chain of an amino acid at a specific position in the Fc region of a binding molecule disclosed herein (e.g., an anti-FIX, anti-FX, or a bispecific anti-FIX/anti-FX antibody disclosed herein).

In some aspects, the specific positions in the Fc region are selected from the group consisting of 239, 248, 254, 258, 273, 279, 282, 284, 286, 287, 289, 297, 298, 312, 324, 326, 330, 335, 337, 339, 350, 355, 356, 359, 360, 361, 375, 383, 384, 389, 398, 400, 413, 415, 418, 422, 435, 440, 441, 442, 443, 446, an insertion between positions 239 and 240, and combinations thereof, wherein the amino acid position numbering is according to the EU index as set forth in Kabat.

In some aspects, the amino acid side chain where the therapeutic or diagnostic moiety is conjugated is a sulfhydryl side chain, for example, the sulfhydryl group of a cysteine amino acid. In some aspects, at least one therapeutic or diagnostic moiety is chemically conjugated to the side chain of an amino acid located at a position outside of the Fc region of a binding molecule disclosed herein (e.g., an anti-FIX, anti-FX, or a bispecific anti-FIX/anti-FX antibody disclosed herein).

In some aspects, all the therapeutic or diagnostic moieties are chemically conjugated to the side chain of an amino acid located at a position outside of the Fc region of a binding molecule disclosed herein (e.g., an anti-FIX, anti-FX, or a bispecific anti-FIX/anti-FX antibody disclosed herein). In some aspects, at least one therapeutic or diagnostic moiety is genetically incorporated into the polypeptide chain of a binding molecule disclosed herein (e.g., an anti-FIX, anti-FX, or a bispecific anti-FIX/anti-FX antibody disclosed herein) using recombinant techniques known in the art.

In some embodiments, the immunoconjugates disclosed herein can comprise a moiety that targets the antibodies or binding molecules disclosed herein to the site of injury. In a particular embodiment, the moiety that targets the antibodies or binding molecules to the site of injury comprises a platelet targeting moiety, e.g., a platelet targeting moiety.

Immunoconjugates disclosed herein comprise at least one of the binding molecules disclosed herein (e.g., an anti-FIX, anti-FX, or a bispecific anti-FIX/anti-FX antibody disclosed herein) which has been derivatized or linked (e.g., chemically or recombinantly) to another molecule (e.g., a peptide, small drug molecule, detectable molecule, etc.). In general, binding molecules disclosed herein (e.g., an anti-FIX, anti-FX, or a bispecific anti-FIX/anti-FX antibody disclosed herein) are derivatized such that their binding to a specific antigen binding site (e.g., an epitope on FIXa and/or an epitope on FXz) is not affected adversely, e.g., by the chemical or enzymatic derivatization, genetic fusion, or labeling.

Accordingly, the binding molecules of the present disclosure are intended to include both intact and modified forms of the binding molecules disclosed herein (e.g., an anti-FIX, anti-FX, or a bispecific anti-FIX/anti-FX antibody disclosed herein). For example, a binding molecules disclosed herein (e.g., an anti-FIX, anti-FX, or a bispecific anti-FIX/anti-FX antibody disclosed herein) or antigen binding portion thereof can be functionally linked (by chemical coupling, genetic fusion, noncovalent association, or otherwise) to one or more other molecular entities, such as a pharmaceutical agent, a detection agent, and/or a protein or peptide that can mediate association of a binding molecule disclosed herein (e.g., an anti-FIX, anti-FX, or a bispecific anti-FIX/anti-FX antibody disclosed herein) with another molecule (such as a streptavidin core region or a polyhistidine tag).

One type of derivatized molecule can be produced by crosslinking two or more molecular entities, e.g., a binding molecule disclosed herein (e.g., an anti-FIX, anti-FX, or a bispecific anti-FIX/anti-FX antibody disclosed herein) and a therapeutic moiety. Suitable crosslinkers include those that are heterobifunctional, i.e., having two distinctly reactive groups separated by an appropriate spacer (e.g., m-maleimidobenzoyl-N-hydroxysuccinimidc ester); or homobifunctional (e.g., disuccinimidyl suberate). Such crosslinkers are available, for example, from Pierce Chemical Company, Rockford, II. Additional bifunctional coupling agents include N-succinimidyl-3-(2-pyridyldithiol) propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene).

Another type of derivatized molecule can be produced by incorporating a detectable label. Useful detection agents include fluorescent compounds (e.g., fluorescein, fluorescein isothiocyanate, rhodamine, 5-dimethylamine-l-napthalenesulfonyl chloride, phycoerythrin, lanthanide phosphors and the like), enzymes that are useful for detection (e.g., horseradish peroxidase, β-galactosidase, luciferase, alkaline phosphatase, glucose oxidase and the like), epitopes recognized by a secondary reporter (e.g., leucine zipper pair sequences, binding sites for secondary antibodies, metal binding domains, epitope tags, etc.). In some aspects, detectable labels can be attached by at least one spacer arm. Spacer arms can be of various lengths to reduce potential steric hindrance.

A binding molecule disclosed herein (e.g., an anti-FIX, anti-FX, or a bispecific anti-FIX/anti-FX antibody) can also be labeled with a radiolabeled, for example, for diagnostic purposes. A binding molecule disclosed herein can also be derivatized with a chemical group, for example a polymer such as polyethylene glycol (PEG), a methyl group, an ethyl group, or a carbohydrate group. These groups can be useful to improve the biological characteristics of the binding molecule (e.g., an anti-FIX, anti-FX, or a bispecific anti-FIX/anti-FX antibody disclosed herein) such as increasing blood serum half-life or to increase tissue binding.

### VI. Nucleic Acids, Expression Vectors, and Cells

The present disclosure also provides nucleic acids encoding the binding molecules disclosed herein, e.g., any of the antibodies or binding molecules disclosed herein. In some aspects, the nucleic acid encodes the heavy and/or light chain variable region of an antibody disclosed herein, or antigen binding portion thereof, or a bispecific or multispecific molecule (e.g., an antibody) disclosed herein. The polynucleotides of the instant disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand. In certain aspects the DNA is a cDNA that is used to produce a non-naturally-occurring recombinant antibody. In some aspects, the RNA is an mRNA that can express a binding molecule disclosed here after administration to a subject in need thereof. In some aspects, the expression of the mRNA can be in in vivo. mRNA expression can also be in vitro or ex vivo. In some aspects, an mRNA encoding a binding molecule disclosed herein (e.g., an anti-FIX antibody, an anti-FX antibody, or a bispecific anti-FIX/anti-FX antibody) can be chemically modified, e.g., to include modified internucleoside linkages (e.g., phosphorothioate), or modified bases (e.g., pseudouridine, thiouridine, etc.).

In certain aspects, the polynucleotides are isolated. In certain aspects, the polynucleotides are substantially pure. In certain aspects the polynucleotides comprise the coding sequence for the mature polypeptide fused in the same reading frame to a polynucleotide (either natural or heterologous) which aids, for example, in expression and secretion of a polypeptide from a host cell (e.g., a leader sequence which functions as a secretory sequence for controlling transport of a polypeptide from the cell). The polypeptide having a leader sequence is a preprotein and can have the leader sequence cleaved by the host cell to form the mature form of the polypeptide. The polynucleotides can also encode for binding molecule proprotein which is the mature protein plus additional amino acid residues, e.g., 5′ amino acid residues. In certain aspects, the polynucleotides are altered to optimize codon usage for a certain host cell.

In certain aspects the polynucleotides comprise the coding sequence for the mature binding molecule, e.g., a binding molecule disclosed herein (e.g., an anti-FIX antibody, an anti-FX antibody, or a bispecific anti-FIX/anti-FX antibody) or an antigen-binding fragment thereof fused in the same reading frame to a heterologous marker sequence that allows, for example, for purification of the encoded polypeptide.

For example, the marker sequence can be a hexa-histidine (His6) tag supplied, for example, by a pQE-9 vector to provide for purification of the mature polypeptide fused to the marker in the case of a bacterial host. In other aspects, the marker sequence can be a hemagglutinin (HA) tag derived, for example, from the influenza hemagglutinin protein, when a mammalian host (e.g., COS-7 cells) is used.

The present disclosure further relates to variants of the described polynucleotides encoding, for example, fragments, analogs, and derivatives of the binding molecules disclosed herein (e.g., anti-FIX antibodies, anti-FX antibodies, or a bispecific anti-FIX/anti-FX antibodies). The polynucleotide variants can contain alterations in the coding regions, non-coding regions, or both. In some aspects the polynucleotide variants contain alterations which produce silent substitutions, additions, or deletions, but do not alter the properties or activities of the encoded polypeptide. In some aspects, nucleotide variants are produced by silent substitutions due to the degeneracy of the genetic code. Polynucleotide variants can be produced for a variety of reasons, e.g., to optimize codon expression for a particular host (change codons in the human mRNA to those preferred by a bacterial host such as E. coli).

In some aspects a DNA sequence encoding a binding molecule disclosed herein or an antigen-binding fragment thereof can be constructed by chemical synthesis, for example, using an oligonucleotide synthesizer. Such oligonucleotides can be designed based on the amino acid sequence of the desired polypeptide and selecting those codons that are favored in the host cell in which the recombinant polypeptide of interest will be produced. Standard methods can be applied to synthesize an isolated polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Also provided is an expression vector or combination of expression vectors comprising one or more of the nucleic acid molecules disclosed herein. Once assembled (by synthesis, site-directed mutagenesis or another method), the polynucleotide sequences encoding a particular isolated polypeptide of interest will be inserted into an expression vector and operatively linked to an expression control sequence appropriate for expression of the protein in a desired host. Proper assembly can be confirmed, for example, by nucleotide sequencing, restriction mapping, and expression of a biologically active polypeptide in a suitable host. As is well known in the art, in order to obtain high expression levels of a transfected gene in a host, the gene must be operatively linked to transcriptional and translational expression control sequences that are functional in the chosen expression host.

In certain aspects, recombinant expression vectors are used to amplify and express DNA encoding the binding molecules disclosed herein. Recombinant expression vectors are replicable DNA constructs which have synthetic or cDNA-derived DNA fragments encoding, for example, a polypeptide chain of a binding molecule disclosed herein or an antigen-binding fragment thereof, operatively linked to suitable transcriptional or translational regulatory elements derived from mammalian, microbial, viral or insect genes.

A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences, as described in detail below. Such regulatory elements can include an operator sequence to control transcription.

The ability to replicate in a host, usually conferred by an origin of replication, and a selection gene to facilitate recognition of transformants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Structural elements intended for use in yeast expression systems include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

The choice of expression control sequence and expression vector will depend upon the choice of host. A wide variety of expression host/vector combinations can be employed. Useful expression vectors for eukaryotic hosts, include, for example, vectors comprising expression control sequences from SV40, bovine papilloma virus, adenovirus and cytomegalovirus. Useful expression vectors for bacterial hosts include known bacterial plasmids, such as plasmids from E. coli, including pCR 1, pBR322, pMB9 and their derivatives, wider host range plasmids, such as M13 and filamentous single-stranded DNA phages.

The present disclosure also provides a cell comprising a nucleic acid or nucleic acids disclosed herein, or an expression vector or vectors disclosed herein. In some aspects, the cell has been transformed with an expression vector or vectors disclosed herein. In some aspects, the cell is a host cell for recombinant expression. For example, in some aspects, the host cell is a prokaryotic cell, a eukaryotic cell, a protist cell, an animal cell, a plant cell, a fungal cell, a yeast cell, an Sf9 cell, a mammalian cell, an avian cell, an insect cell, a CHO cell, a HEK cell, or a COS cell. Prokaryotes include gram negative or gram positive organisms, for example E. coli or bacilli. Higher eukaryotic cells include established cell lines of mammalian origin. Cell-free translation systems can also be employed. Appropriate cloning and expression vectors for use with bacterial, fungal, yeast, and mammalian cellular hosts are described by Pouwels et al. (Cloning Vectors: A Laboratory Manual, Elsevier, N.Y., 1985), the relevant disclosure of which is hereby incorporated by reference. Additional information regarding methods of protein production, including antibody production, can be found, e.g., in U.S. Publ. No. 2008/0187954, U.S. Pat. Nos. 6,413,746 and 6,660,501, and Int'l Pat. Publ. No. WO 04009823, each of which is hereby incorporated by reference in its entirety.

Expression of recombinant proteins in mammalian cells can be performed because such proteins are generally correctly folded, appropriately modified and completely functional. Examples of suitable mammalian host cell lines include HEK-293 and HEK-293T, the COS-7 lines of monkey kidney cells, described by Gluzman (Cell 23:175, 1981), and other cell lines including, for example, L cells, C127, 3T3, Chinese hamster ovary (CHO), NSO, HeLa, and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences. Baculovirus systems for production of heterologous proteins in insect cells are reviewed by Luckow & Summers, BioTechnology 6:47 (1988).

Binding molecules disclosed herein or antigen-binding fragments thereof, produced by a transformed host can be purified according to any suitable method. Such standard methods include, for example, chromatography (e.g., ion exchange, affinity and sizing column chromatography), centrifugation, differential solubility, or by any other standard technique for protein purification. Affinity tags such as hexahistidine, maltose binding domain, influenza coat sequence, glutathione-S-transferase, etc., can be attached to the protein to allow easy purification by passage over an appropriate affinity column. Isolated proteins can also be physically characterized using, for example, proteolysis, nuclear magnetic resonance or x-ray crystallography.

For example, supernatants from systems which secrete recombinant protein into culture media can be first concentrated using a commercially available protein concentration filter, for example, an AMICON® or Millipore PELLICON® ultrafiltration unit. Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed.

Suitable cation exchangers include various insoluble matrices comprising sulfopropyl or carboxymethyl groups. Finally, one or more reversed-phase high performance liquid chromatography (RP-HPLC) steps employing hydrophobic RP-HPLC media, e.g., silica gel having pendant methyl or other aliphatic groups, can be employed to further purify a binding molecule disclosed herein or an antigen binding portion thereof. Some or all of the foregoing purification steps, in various combinations, can also be employed to provide a homogeneous recombinant protein.

A recombinant binding molecule disclosed herein or antigen binding portion thereof produced in bacterial culture can be isolated, for example, by initial extraction from cell pellets, followed by one or more concentration, salting-out, aqueous ion exchange or size exclusion chromatography steps. High performance liquid chromatography (HPLC) can be employed for final purification steps. Microbial cells employed in expression of a recombinant protein can be disrupted by any convenient method, including freeze-thaw cycling, sonication, mechanical disruption, or use of cell lysing agents.

Methods known in the art for purifying antibodies and other proteins also include, for example, those described in U.S. Pat. Publ. Nos. US20080312425, US20080177048, and US20090187005, each of which is hereby incorporated by reference in its entirety.

### VII. Methods of Making and Characterization

The present disclosure also provides methods of preparing the binding molecules disclosed herein, e.g., antibodies. In some aspects, the method of producing (i) an anti-FIX antibody, or antigen binding portion thereof, (ii) an anti-FX antibody, or antigen binding portion thereof, or (iii) a bispecific molecule disclosed herein comprises expressing the antibody antigen binding portion thereof or bispecific molecule in a cell (e.g., a host cell) and isolating the antibody, antigen binding portion thereof, or bispecific molecule from the cell.

The disclosure provides a method of producing a bispecific molecule, comprising culturing a host cell disclosed herein under conditions that allow the expression of the bispecific molecule. Also provides is a method of producing a bispecific molecule disclosed herein further comprising conditions that enhance heterodimerization.

Binding molecules disclosed herein (e.g., anti-FIX antibodies, anti-FX antibodies, or bispecific anti-FIX/anti-FX antibodies) can be prepared according to methods known in the art. For example, binding molecules disclosed herein (e.g., anti-FIX antibodies, anti-FX antibodies, or bispecific anti-FIX/anti-FX antibodies) can be generated using hybridoma methods, such as those described by Kohler & Milstein (1975) Nature 256:495.

Using the hybridoma method, a mouse, hamster, or other appropriate host animal, is immunized as described above to elicit the production by lymphocytes of antibodies that will specifically bind to an immunizing antigen. Lymphocytes can also be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen as determined by immunoprecipitation, immunoblotting, or by an in vitro binding assay (e.g. radioimmunoassay (RIA); enzyme-linked immunosorbent assay (ELISA)) can then be propagated either in in vitro culture using standard methods (Goding, Monoclonal Antibodies: Principles and Practice, Academic Press, 1986) or in vivo as ascites tumors in an animal. The monoclonal antibodies can then be purified from the culture medium or ascites fluid as described for polyclonal antibodies above.

Binding molecules disclosed herein (e.g., anti-FIX antibodies, anti-FX antibodies, or bispecific anti-FIX/anti-FX antibodies) can also be made using recombinant DNA methods as described in U.S. Pat. No. 4,816,567. The polynucleotides encoding a monoclonal antibody are isolated from mature B-cells or hybridoma cell, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells.

Also, recombinant monoclonal antibodies or molecules comprising antigen-binding fragments thereof of the desired species can be isolated from phage display libraries expressing CDRs of the desired species as described (McCafferty et al., Nature 348:552-554 (1990); Clarkson et al., Nature 352:624-628 (1991); and Marks et al., J. Mol. Biol. 222:581-597 (1991)).

The polynucleotide(s) encoding binding molecules disclosed herein (e.g., anti-FIX antibodies, anti-FX antibodies, or bispecific anti-FIX/anti-FX antibodies) can further be modified in a number of different manners using recombinant DNA technology to generate alternative binding molecules. In some aspects, the constant domains of the light and heavy chains of, for example, a mouse monoclonal antibody can be substituted (1) for those regions of, for example, a human antibody to generate a chimeric antibody or (2) for a non-immunoglobulin polypeptide to generate a fusion antibody. In some aspects, the constant regions are truncated or removed to generate the desired antibody fragment of a monoclonal antibody. Site-directed or high-density mutagenesis of the variable region can be used to optimize specificity, affinity, etc. of a monoclonal antibody.

In certain aspects, a binding molecule disclosed herein (e.g., an anti-FIX antibody, anti-FX antibody, or bispecific anti-FIX/anti-FX antibody) is a human antibody or antigen-binding fragment thereof. Human antibodies can be directly prepared using various techniques known in the art. Immortalized human B lymphocytes immunized in vitro or isolated from an immunized individual that produce an antibody directed against a target antigen can be generated (See, e.g., Cole et al., Monoclonal Antibodies and Cancer Therapy, Alan R. Liss, p. 77 (1985); Boemer et al., J. Immunol. 147:86-95 (1991); and U.S. Pat. No. 5,750,373). One or more cDNAs encoding the antibody in the immortalized B lymphocyte can then be prepared and inserted into an expression vector and/or a heterologous host cell for expression of a non-naturally-occurring recombinant version of the antibody.

Also, a binding molecule disclosed herein (e.g., an anti-FIX antibody, anti-FX antibody, or bispecific anti-FIX/anti-FX antibody) can be selected from a phage library, where that phage library expresses human antibodies or fragments thereof as fusion proteins with heterologous phage proteins, as described, for example, in Vaughan et al., Nat. Biotech. 14:309-314 (1996); Sheets et al., Proc. Natl. Acad. Sci. 95:6157-6162 (1998); Hoogenboom and Winter, J. Mol. Biol. 227:381 (1991), and Marks et al., J. Mol. Biol. 222:581 (1991)). Techniques for the generation and use of antibody phage libraries are also described in U.S. Pat. Nos. 5,969,108, 6,172,197, 5,885,793, 6,521,404; 6,544,731; 6,555,313; 6,582,915; 6,593,081; 6,300,064; 6,653,068; 6,706,484; and 7,264,963, each of which is incorporated by reference in its entirety.

Affinity maturation strategies and chain shuffling strategies (Marks et al., BioTechnology 10:779-783 (1992), incorporated by reference in its entirety) are known in the art and can be employed to generate high affinity human antibodies or antigen-binding fragments thereof.

In some aspects, a binding molecule disclosed herein (e.g., an anti-FIX antibody, anti-FX antibody, or bispecific anti-FIX/anti-FX antibody) can be a humanized antibody. Methods for engineering, humanizing or resurfacing non-human or human antibodies can also be used and are well known in the art. A humanized, resurfaced or similarly engineered antibody can have one or more amino acid residues from a source that is non-human, e.g., but not limited to, mouse, rat, rabbit, non-human primate or other mammal. These non-human amino acid residues are replaced by residues that are often referred to as “import” residues, which are typically taken from an “import” variable, constant or other domain of a known human sequence. Such imported sequences can be used to reduce immunogenicity or reduce, enhance or modify binding, affinity, on-rate, off-rate, avidity, specificity, half-life, or any other suitable characteristic, as known in the art. In general, the CDR residues are directly and most substantially involved in influencing binding to a specific anti binding site (e.g., an epitope). Accordingly, part or all of the non-human or human CDR sequences are maintained while the non-human sequences of the variable and constant regions can be replaced with human or other amino acids. In certain aspects, human CDRs are inserted into non-human antibody scaffolds to make an antibody with reduced immunogenicity in an animal model system, e.g., a “murinized” antibody.

In some aspects, a binding molecule disclosed herein (e.g., an anti-FIX antibody, anti-FX antibody, or bispecific anti-FIX/anti-FX antibody) can be humanized, resurfaced, or engineered with retention of high affinity for the antigen binding site (e.g., an epitope) and other favorable biological properties. To achieve this goal, humanized (or human), engineered, or resurfaced binding molecules can be optionally prepared by a process of analysis of the parental sequences and various conceptual humanized and engineered products using three-dimensional models of the parental, engineered, and humanized sequences. Three-dimensional immunoglobulin models are commonly available and are familiar to those skilled in the art.

Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, framework residues can be selected and combined from the consensus and import sequences so that the desired antibody characteristic, such as increased affinity for the target antigen(s), is achieved.

Humanization, resurfacing or engineering of a binding molecule disclosed herein (e.g., an anti-FIX antibody, anti-FX antibody, or bispecific anti-FIX/anti-FX antibody) can be performed using any known method, such as but not limited to those described in, Jones et al., Nature 321:522 (1986); Riechmann et al., Nature 332:323 (1988); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), U.S. Pat. Nos. 5,639,641, 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; 4,816,567, 7,557,189; 7,538,195; and 7,342,110; WO90/14443; WO90/14424; WO90/14430; and EP229246, each of which is entirely incorporated herein by reference, including the references cited therein.

In certain an antibody fragment obtained from a binding molecule disclosed herein (e.g., an anti-FIX antibody, anti-FX antibody, or bispecific anti-FIX/anti-FX antibody) is provided. Various techniques are known for the production of antibody fragments. Traditionally, these fragments are derived via proteolytic digestion of intact antibodies (for example Morimoto et al., J. Biochem. Biophy. Methods 24:107-117 (1993); Brennan et al., Science, 229:81 (1985)).

In certain aspects, antibody fragments are produced recombinantly. Fab, Fv, and scFv antibody fragments can all be expressed in and secreted from E. coli or other host cells, thus allowing the production of large amounts of these fragments. Such antibody fragments can also be isolated from the antibody phage libraries discussed above. The antibody fragments can also be linear antibodies as described in U.S. Pat. No. 5,641,870. Other techniques for the production of antibody fragments will be apparent to the skilled practitioner.

Techniques can be adapted for the production of single-chain antibodies specific to the same epitope(s) as the binding molecules disclosed herein (e.g., anti-FIX antibodies, anti-FX antibodies, or bispecific anti-FIX/anti-FX antibodies). In addition, methods can be adapted for the construction of Fab expression libraries (see, e.g., Huse et al., Science 246:1275-1281 (1989)) to allow rapid and effective identification of monoclonal Fab fragments with the desired specificity for FIX and/or FX, or derivatives, fragments, analogs or homologs thereof. Antibody fragments can be produced by techniques in the art including, but not limited to: (a) a F(ab′)₂ fragment produced by pepsin digestion of an antibody molecule; (b) a Fab fragment generated by reducing the disulfide bridges of an F(ab′)₂ fragment, (c) a Fab fragment generated by the treatment of the antibody molecule with papain and a reducing agent, and (d) Fv fragments.

The binding molecules disclosed herein and antigen binding portion thereof, variants, or derivatives thereof, can be assayed for immunospecific binding by any method known in the art. The immunoassays that can be used include but are not limited to competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds, (1994) Current Protocols in Molecular Biology (John Wiley & Sons, Inc., NY) Vol. 1, which is incorporated by reference herein in its entirety).

The binding activity of a given lot of a binding molecule disclosed herein, antigen binding portion thereof, variant, or derivative thereof, can be determined according to well-known methods. Those skilled in the art will be able to determine operative and optimal assay conditions for each determination by employing routine experimentation.

Methods and reagents suitable for determination of binding characteristics of a binding molecule disclosed herein or an antigen binding portion thereof, are known in the art and/or are commercially available. Equipment and software designed for such kinetic analyses are commercially available (e.g., BIACORE®, BIAevaluation software, GE Healthcare; KinExa Software, Sapidyne Instruments).

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); and in Ausubel et al. (1989) Current Protocols in Molecular Biology (John Wiley and Sons, Baltimore, Md.).

General principles of antibody engineering are set forth in Borrebaeck, ed. (1995) Antibody Engineering (2nd ed.; Oxford Univ. Press). General principles of protein engineering are set forth in Rickwood et al., eds. (1995) Protein Engineering, A Practical Approach (IRL Press at Oxford Univ. Press, Oxford, Eng.). General principles of antibodies and antibody-hapten binding are set forth in: Nisonoff (1984) Molecular Immunology (2nd ed.; Sinauer Associates, Sunderland, Mass.); and Steward (1984) Antibodies, Their Structure and Function (Chapman and Hall, New York, N.Y.). Additionally, standard methods in immunology known in the art and not specifically described are generally followed as in Current Protocols in Immunology, John Wiley & Sons, New York; Stites et al., eds. (1994) Basic and Clinical Immunology (8th ed; Appleton & Lange, Norwalk, Conn.) and Mishell and Shiigi (eds) (1980) Selected Methods in Cellular Immunology (W.H. Freeman and Co., NY).

Standard reference works setting forth general principles of immunology include Current Protocols in Immunology, John Wiley & Sons, New York; Klein (1982) J., Immunology: The Science of Self-Nonself Discrimination (John Wiley & Sons, NY); Kennett et al., eds. (1980) Monoclonal Antibodies, Hybridoma: A New Dimension in Biological Analyses (Plenum Press, NY); Campbell (1984) “Monoclonal Antibody Technology” in Laboratory Techniques in Biochemistry and Molecular Biology, ed. Burden et al., (Elsevier, Amsterdam); Goldsby et al., eds. (2000) Kuby Immunology (4th ed.; H. Freemand & Co.); Roitt et al. (2001) Immunology (6th ed.; London: Mosby); Abbas et al. (2005) Cellular and Molecular Immunology (5th ed.; Elsevier Health Sciences Division); Kontermann and Dubel (2001) Antibody Engineering (Springer Verlag); Sambrook and Russell (2001) Molecular Cloning: A Laboratory Manual (Cold Spring Harbor Press); Lewin (2003) Genes VIII (Prentice Hall 2003); Harlow and Lane (1988) Antibodies: A Laboratory Manual (Cold Spring Harbor Press); Dieffenbach and Dveksler (2003) PCR Primer (Cold Spring Harbor Press).

### VIII. Pharmaceutical Compositions

The present disclosure also provides pharmaceutical compositions comprising, e.g., (i) an antibody or antigen binding portion thereof disclosed herein, a bispecific molecule (e.g., a bispecific antibody) disclosed herein, or an immunoconjugate disclosed herein, and (ii) a carrier.

In particular, the disclosure provides pharmaceutical compositions (e.g., therapeutic or diagnostic compositions) comprising,

(1) at least one therapeutically or diagnostically active component selected from the group consisting of
(i) binding molecules disclosed herein, e.g., anti-FIX antibodies or antigen binding portions thereof, anti-FX antibodies or antigen binding portions thereof, bispecific molecules comprising an anti-FIX specificity and/or an anti-FX specificity;
(ii) derivatives thereof, for example, immunoconjugates, fusion proteins, or derivatives with heterologous moieties that confer a desired property to the binding molecules disclosed herein (e.g., extended plasma half life);
(iii) polynucleotides encoding the binding molecules of (i) and/or the derivatives of (ii);
(iv) vectors comprising the polynucleotides of (iii);
(v) cells comprising the polynucleotides of (iii) or vectors of (iv); or,
(vi) combinations thereof; and,
(2) one or more carriers, excipients and/or diluents.

The pharmaceutical compositions disclosed herein may be suitable for veterinary uses or pharmaceutical uses in humans. The pharmaceutical compositions disclosed herein typically comprise one or more therapeutically or diagnostically active components described herein and one or more carriers, excipients and/or diluents. The form of the pharmaceutical composition (e.g., dry powder, liquid formulation, etc.) and the excipients, diluents, and/or carriers used will depend upon the intended uses of the compositions, therapeutic or diagnostic uses, and the mode of administration.

The pharmaceutical compositions comprising the therapeutically or diagnostically active components described herein are for use in, but not limited to, diagnosing, detecting, or monitoring a disorder, in preventing, treating, managing, or ameliorating a disorder or one or more symptoms thereof, and/or in research.

For therapeutic uses, the pharmaceutical compositions may be supplied as part of a sterile pharmaceutical composition that includes a pharmaceutically acceptable carrier. This pharmaceutical composition can be in any suitable form (depending upon the desired method of administering it to a patient). Methods to accomplish administration are known to those of ordinary skill in the art. The pharmaceutical composition can be administered to a patient by a variety of routes such as orally, transdermally, subcutaneously, intranasally, intravenously, intramuscularly, intratumorally, intrathecally, topically or locally. The most suitable route for administration in any given case will depend on the particular therapeutically or diagnostically active components, the subject, and the nature and severity of the disease and the physical condition of the subject. Typically, the pharmaceutical composition will be administered subcutaneously.

Pharmaceutical compositions can be conveniently presented in unit dosage forms containing a predetermined amount of a therapeutically or diagnostically active component described herein per dose. The quantity of the therapeutically or diagnostically active component included in a unit dose will depend on the disease being treated or diagnosed, as well as other factors as are well known in the art. Such unit dosages may be in the form of a lyophilized dry powder containing an amount of the therapeutically or diagnostically active component suitable for a single administration, or in the form of a liquid. Dry powder unit dosage forms may be packaged in a kit with a syringe, a suitable quantity of diluent and/or other components useful for administration. Unit dosages in liquid form may be conveniently supplied in the form of a syringe pre-filled with a quantity of the therapeutically or diagnostically active component suitable for a single administration.

The pharmaceutical compositions may also be supplied in bulk form containing quantities of the therapeutically or diagnostically active component suitable for multiple administrations.

The pharmaceutical compositions may also be included in a container, pack, or dispenser together with instructions for administration.

Pharmaceutical compositions may be prepared for storage as lyophilized formulations or aqueous solutions by mixing a therapeutically or diagnostically active component described herein having the desired degree of purity with optional pharmaceutically-acceptable carriers, excipients, or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington's Pharmaceutical Sciences, 16th edition (Osol, ed. 1980) and Remington: The Science and Practice of Pharmacy, 22nd Edition (Edited by Allen, Loyd V. Jr., 2012). Such additives should be nontoxic to the recipients at the dosages and concentrations employed.

Solutions or suspensions used for intradermal or subcutaneous application typically include one or more of the following components: a sterile diluent such as water for injection, saline solution, fixed oils, polyethylene glycols, glycerine, propylene glycol, or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates, or phosphates; and agents for the adjustment of tonicity such as sodium chloride or dextrose. The pH can be adjusted with acids or bases, such as hydrochloric acid or sodium hydroxide. Such preparations may be enclosed in ampoules, disposable syringes, or multiple dose vials made of glass or plastic.

Pharmaceutical compositions suitable for injection include sterile aqueous solutions or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.), or phosphate buffered saline. In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants.

Preservatives may be added to retard microbial growth, and can be added in amounts ranging from about 0.2%-1% (w/v). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol and mannitol.

Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinositol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, α-monothioglycerol and sodium thiosulfate; low molecular weight polypeptides (e.g., peptides of 10 residues or fewer); proteins such as human serum albumin, bovine serum albumin, gelatin or immunoglobulins; hydrophilic polymers, such as polyvinylpyrrolidone monosaccharides, such as xylose, mannose, fructose, glucose; disaccharides such as lactose, maltose, sucrose and trehalose; and trisaccacharides such as raffinose; and polysaccharides such as dextran.

Buffering agents help to maintain the pH in the range which stabilizes the protein. They may be present at a wide variety of concentrations, but will typically be present in concentrations ranging from about 2 mM to about 50 mM.

Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium gluconate mixture, gluconic acid-sodium hydroxide mixture, gluconic acid-potassium gluconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Additional miscellaneous excipients include bulking agents (e.g., starch), chelating agents (e.g., EDTA), antioxidants (e.g., ascorbic acid, methionine, vitamin E), and co-solvents.

### IX. Treatment and Diagnostic Methods

The present disclosure also provides treatment and diagnostic methods comprising the use of the binding molecules disclosed herein (e.g., anti-FIX antibodies, anti-FX antibodies, or bispecific anti-FIX/anti-FX antibodies) or other compositions of the present disclosure (e.g., nucleic acids, vectors, cells).

(a) Therapeutic Uses

In one aspect, the binding molecules disclosed herein (e.g., anti-FIX antibodies, anti-FX antibodies, or bispecific anti-FIX/anti-FX antibodies) or other compositions of the present disclosure (e.g., nucleic acids, vectors, cells) may be used to prevent, treat, or reverse a coagulation or bleeding disorder. In another aspect, the method comprises administering to a subject in need thereof, a binding molecule disclosed herein (e.g., anti-FIX antibodies, anti-FX antibodies, or bispecific anti-FIX/anti-FX antibodies) or other composition of the present disclosure (e.g., nucleic acids, vectors, cells). The subject can be a mammal, such as but not limited to a human, a mouse, a rat, a guinea pig, a domesticated animal, such as, but not limited to, a cow, a horse, a sheep, a pig, a goat, a cat, a dog, a hamster, a donkey. In one aspect, the subject is a human.

In various aspects, the coagulation or bleeding disorder is caused by the absence of a coagulation factor. One of skill in the art would appreciate the types of coagulation or bleeding disorders associated with the absence of a coagulation factor. In some aspects, the coagulation or bleeding disorder may be hemophilia or von Willebrand disease. In another aspect, the coagulation or bleeding disorder is hemophilia A or acquired hemophilia. In a particular aspect, the coagulation or bleeding disorder is hemophilia A. In another aspect, the coagulation or bleeding disorder is acquired hemophilia where the subject no longer produces FVIII.

In various aspects, the binding molecules disclosed herein (e.g., anti-FIX antibodies, anti-FX antibodies, or bispecific anti-FIX/anti-FX antibodies) or other compositions of the present disclosure (e.g., nucleic acids, vectors, cells) may be administered to a subject with mild hemophilia A, moderate hemophilia A, or severe hemophilia A. In another aspect, the binding molecules disclosed herein (e.g., anti-FIX antibodies, anti-FX antibodies, or bispecific anti-FIX/anti-FX antibodies) or other compositions of the present disclosure (e.g., nucleic acids, vectors, cells) may be administered to a subject with factor plasma levels of 6% to 30%, 2% to 5%, or 1% or less.

In some aspects, the binding molecules disclosed herein (e.g., anti-FIX antibodies, anti-FX antibodies, or bispecific anti-FIX/anti-FX antibodies) or other compositions of the present disclosure (e.g., nucleic acids, vectors, cells) may be administered to a subject with hemophilia A or suspected of having hemophilia A when there is an external wound on the subject. In another aspect, the binding molecules disclosed herein (e.g., anti-FIX antibodies, anti-FX antibodies, or bispecific anti-FIX/anti-FX antibodies) or other compositions of the present disclosure (e.g., nucleic acids, vectors, cells) may be administered to a subject with hemophilia A or suspected of having hemophilia A with an existing external wound on the subject. In another aspect, the binding molecules disclosed herein (e.g., anti-FIX antibodies, anti-FX antibodies, or bispecific anti-FIX/anti-FX antibodies) or other compositions of the present disclosure (e.g., nucleic acids, vectors, cells) may be administered to a subject with an external wound until the wound has healed. In some aspects, the wound may include, but not limited to, an abrasion, a laceration, a puncture, or an avulsion.

In some aspects, the binding molecules disclosed herein (e.g., anti-FIX antibodies, anti-FX antibodies, or bispecific anti-FIX/anti-FX antibodies) or other compositions of the present disclosure (e.g., nucleic acids, vectors, cells) may be administered to a subject with hemophilia, A or suspected of having hemophilia A, prior to, during, or after surgery, a serious injury, or dental work.

In some aspects, the binding molecules disclosed herein (e.g., anti-FIX antibodies, anti-FX antibodies, or bispecific anti-FIX/anti-FX antibodies) or other compositions of the present disclosure (e.g., nucleic acids, vectors, cells) may be administered to a subject with hemophilia A, or suspected of having hemophilia A, and has experienced spontaneous bleeding. In another aspect, the binding molecules disclosed herein (e.g., anti-FIX antibodies, anti-FX antibodies, or bispecific anti-FIX/anti-FX antibodies) or other compositions of disclosure (e.g., nucleic acids, vectors, cells) may be administered to a subject with hemophilia A, or suspected of having hemophilia A, and has experienced bleeding once, twice, or more times in a week.

In various aspects, the binding molecules disclosed herein (e.g., anti-FIX antibodies, anti-FX antibodies, or bispecific anti-FIX/anti-FX antibodies) or other compositions of the present disclosure (e.g., nucleic acids, vectors, cells) may be administered to a subject of any age group suffering from, or suspected of having hemophilia A. In some aspects, the binding molecules disclosed herein (e.g., anti-FIX antibodies, anti-FX antibodies, or bispecific anti-FIX/anti-FX antibodies) or other compositions of the present disclosure (e.g., nucleic acids, vectors, cells) may be administered to a child of 1, 2, 3, 4, 5, 6, 7, 8, 9, 10, 11, 12, 13, 14, 15, 16, or 17 years of age suffering from, or suspected of having hemophilia A. In another aspect, the binding molecules disclosed herein (e.g., anti-FIX antibodies, anti-FX antibodies, or bispecific anti-FIX/anti-FX antibodies) or other compositions of the present disclosure (e.g., nucleic acids, vectors, cells) thereof may be administered to an infant suffering from or suspected of having hemophilia A.

In yet another aspects, the binding molecules disclosed herein (e.g., anti-FIX antibodies, anti-FX antibodies, or bispecific anti-FIX/anti-FX antibodies) or other compositions of the present disclosure (e.g., nucleic acids, vectors, cells) may be administered to a subject who is an infant of 0, 1, 2, 3, 4, 5, 6, 7, 8, 9, 10, 11, or 12 months of age suffering from, or suspected of having hemophilia A.

In some aspects, the binding molecules disclosed herein (e.g., anti-FIX antibodies, anti-FX antibodies, or bispecific anti-FIX/anti-FX antibodies) or other compositions of the present disclosure (e.g., nucleic acids, vectors, cells) are administered to a subject at an early age before the first episode of bleeding.

In other aspects, administering the binding molecules disclosed herein (e.g., anti-FIX antibodies, anti-FX antibodies, or bispecific anti-FIX/anti-FX antibodies) or other compositions of the present disclosure (e.g., nucleic acids, vectors, cells) before the first episode of bleeding protects against further bleeding and development of joint damage in the future.

In some embodiments, administering a binding molecule disclosed herein (e.g., anti-FIX antibodies, anti-FX antibodies, or bispecific anti-FIX/anti-FX antibodies) or another composition of the present disclosure (e.g., nucleic acids, vectors, cells) to subjects may have the following effects, but is not limited to, hemostasis, reduced pain, and improved mobility.

Also provided is method of promoting FX activation in a subject in need thereof, comprising administering to the subject a therapeutically effective amount of an antibody, bispecific molecule, immunoconjugate, pharmaceutical composition, nucleic acid (e.g., DNA or mRNA), vector, or cell (e.g., a host cell) disclosed herein, or a combination thereof.

Also provided is a method of reducing the frequency or degree of a bleeding episode in a subject in need thereof, comprising administering to the subject an effective amount of an antibody, bispecific molecule, immunoconjugate, pharmaceutical composition, nucleic acid (e.g., DNA or mRNA), vector, or cell (e.g., a host cell) disclosed herein, or a combination thereof.

In some aspects, the subject has developed, has a tendency to develop, and is at risk to develop an inhibitor against Factor VIII (“FVIII”). In some aspects, the inhibitor against FVIII is a neutralizing antibody against FVIII. In some aspect, the subject is undergoing treatment with FVIII or is a candidate for treatment with FVIII, e.g., FVIII replacement therapy.

In some aspects, the bleeding episode is the result of hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, bleeding in the illiopsoas sheath, or any combinations thereof.,

The present disclosure also provides a method of treating a blood coagulation disorder in a subject in need thereof, comprising administering to the subject an effective amount of a bispecific molecule, immunoconjugate, pharmaceutical composition, nucleic acid (e.g., DNA or mRNA), vector, or cell (e.g., a host cell) disclosed herein, or a combination thereof.

In some aspects, the blood coagulation disorder is hemophilia A or hemophilia B. In some aspects, the subject is a human subject.

In some aspects, the subject is undergoing or has undergone FVIII replacement therapy. In some aspects, the bispecific molecule is administered in combination with a hemophilia therapy. In some aspects, the hemophilia therapy is a FVIII replacement therapy. In some aspects, the bispecific molecule is administered before, during or after administration of the hemophilia therapy. In some aspects, the bispecific molecule is administered intravenously or subcutaneously.

In some aspects, administration of the bispecific molecule reduces the frequency of break-through bleeding episodes, spontaneous bleeding episodes, or acute bleeding. In some aspects, administration of the bispecific molecule reduces the annualized bleed rate by 5%, 10%, 20%, 30%, or 50%.

The binding molecules disclosed herein (e.g., anti-FIX antibodies, anti-FX antibodies, or bispecific anti-FIX/anti-FX antibodies) may be administered by any route appropriate to the condition to be treated. The binding molecules disclosed herein (e.g., anti-FIX antibodies, anti-FX antibodies, or bispecific anti-FIX/anti-FX antibodies) will typically be administered parenterally, i.e., infusion, subcutaneous, intramuscular, intravenous, or intradermal. In some aspects, the binding molecules disclosed herein (e.g., anti-FIX antibodies, anti-FX antibodies, or bispecific anti-FIX/anti-FX antibodies) are administered subcutaneously.

In certain aspects, the binding molecules disclosed herein (e.g., anti-FIX antibodies, anti-FX antibodies, or bispecific anti-FIX/anti-FX antibodies) are administered intermittently or discontinuously. In various aspects, dose levels of the binding molecules disclosed herein (e.g., anti-FIX antibodies, anti-FX antibodies, or bispecific anti-FIXa/anti-FX antibodies), for example, administered via injection, such as subcutaneous injection range from about 0.0001 mg/kg to about 100 mg/kg bodyweight.

In some aspects, the binding molecules disclosed herein (e.g., anti-FIX antibodies, anti-FX antibodies, or bispecific anti-FIX/anti-FX antibodies) are administered until disease progression or unacceptable toxicity.

(b) Diagnostic Uses

The present disclosure further provides diagnostic methods useful for diagnosis of diseases characterized by the abnormal or defective expression of clotting factors, such as FIX and FX. In some aspects, the diagnosis involves measuring the expression level of FIX (e.g., FIXa) and/or FX (e.g., FXz) in tissues or body fluids from an individual and comparing the measured expression level with a standard expression level of FIX (e.g., FIXa) and/or FX (e.g., FXz) in normal tissue or body fluid, whereby an increase or decrease in the expression level compared to the standard is indicative of a disorder.

The binding molecules of the instant disclosure and antigen-binding fragments, variants, and derivatives thereof, can be used to assay FIX (e.g., FIXa) and/or FX (e.g., FXz) protein levels in a biological sample using classical immunohistological methods known to those of skill in the art (e.g., see Jalkanen, et al., J. Cell. Biol. 101:976-985 (1985); Jalkanen et al., J. Cell Biol. 105:3087-3096 (1987)).

Other antibody-based methods useful for detecting FIX (e.g., FIXa) and/or FX (e.g., FXz) protein expression include immunoassays, such as the enzyme linked immunosorbent assay (ELISA), immunoprecipitation, or Western blotting. Suitable assays are described in more detail elsewhere herein.

The phrase assaying the expression level of FIX (e.g., FIXa) or FX (e.g., FXz) polypeptide refers both to qualitatively or quantitatively measuring or estimating the level of FIX (e.g., FIXa) or FX (e.g., FXz) polypeptide in a first biological sample either directly (e.g., by determining or estimating absolute protein level) or relatively (e.g., by comparing to the disease associated polypeptide level in a second biological sample).

FIX (e.g., FIXa) or FX (e.g., FXz) polypeptide expression level in the first biological sample can be measured or estimated and compared to a standard FIX (e.g., FIXa) or FX (e.g., FXz) polypeptide level, the standard being taken from a second biological sample obtained from an individual not having the disorder or being determined by averaging levels from a population of individuals not having the disorder. As will be appreciated in the art, once the “standard” FIX (e.g., FIXa) or FX (e.g., FXz) polypeptide level is known, it can be used repeatedly as a standard for comparison.

The present disclosure provides a method of measuring a level of activated FIX in a subject in need thereof comprising contacting an anti-FIXa antibody disclosed herein, or antigen binding portion thereof, with a sample obtained from the subject under suitable conditions and measuring the binding of the anti-FIXa antibody or antigen binding portion thereof to FIXa in the sample.

In one embodiment, the anti-FIXa antibody or antigen binding portion thereof in Class I can be used to measure the level of activated FIX in a tenase complex compared to free FIXa or FIX zymogen. In another embodiment, the anti-FIXz antibody or antigen binding portion thereof in Class IV can be used to measure the level of FIX zymogen compared to free FIXa or FIXa in a tenase complex. In other embodiments, the anti-FIXa antibody or antigen binding portion thereof in Class II can be used to measure the level of free FIXs compared to FIXa in a tenase complex or FIX zymogen. In some embodiments, the anti-FIXa antibody or antigen binding portion thereof in Class III can be used to measure the level of activated FIX (i.e., free FIXa and FIXa in a tenase complex) compared to FIX zymogen.

Also provided is a method of measuring zymogen FX (FXz) in a subject in need thereof comprising contacting an anti-FXz antibody, or antigen binding portion thereof, in Class V with a sample obtained from the subject under suitable conditions and measuring the binding of the anti-FX antibody or antigen binding portion thereof to FXz in the sample. In other embodiments, the anti-FXa antibody, or antigen binding portion thereof, in Class VI can be used to measure the level of activated FX compared to FX zymogen. In some aspects, the sample is blood or serum.

### X. Combination Treatments

The binding molecules disclosed herein (e.g., anti-FIX antibodies, anti-FX antibodies, or bispecific anti-FIX/anti-FX antibodies) can be administered as the sole active agent or can also be administered in combination with one or more additional medicaments or therapeutic agents useful in the treatment of various diseases, e.g., as a combination therapy. For example, the additional agent can be a therapeutic agent art-recognized as being useful to treat the disease or condition being treated by the bispecific antibody provided herein. The combination can also include more than one additional agent, e.g., two or three additional agents.

In certain embodiments, the additional medicament or therapeutic agent is an agent routinely used in the treatment of coagulation or bleeding disorders. Those skilled in the art will recognize routine therapeutic agents. In some embodiments, the additional therapeutic agent is a hemophilia therapy agent. An exemplary hemophilia therapy agent includes, but is not limited to factor concentrate replacement therapy. In particular embodiments, cofactor replacement therapy is FVIII replacement therapy. Those skilled in the art would know that replacement therapy may be plasma-derived and/or recombinant FVIII replacement. In some embodiments, the FVIII replacement therapy is recombinant FVIII. In another embodiment, the FVIII replacement therapy is plasma-derived FVIII. In various embodiments, the additional medicament or therapeutic agent may include, for example, desmopressin acetate, clot-preserving medications (e.g., antifibrinolytics), or fibrin sealants.

In certain embodiments, the additional medicament or therapeutic agent can be administered to a subject, before, during, or after administration of the antibodies described herein. In various embodiments, the additional medicament or therapeutic agent and the antibodies provided herein may be administered on the same dosing schedule. In some embodiments, the additional medicament or therapeutic agent is administered concurrently with the antibodies described herein.

In various embodiments, the additional medicament or therapeutic agent is administered prophylactically or on a need basis. In another embodiment, the additional medicament or therapeutic agent is administered as the primary treatment. In other embodiments, the additional medicament or therapeutic agent is administered as the secondary treatment.

In some embodiments, when a binding molecule disclosed herein (e.g., an anti-FIX antibody, and anti-FX antibody, a bispecific anti-FIX/anti-FX antibody, or an immunoconjugate) is administered with or adjunctive to standards of care, the treatment with the binding molecule disclosed herein can be initiated prior to the commencement of standard therapy, for example a day, several days, a week, several weeks, a month or even several months before the commencement of standard therapy.

### XI. Kits

The present disclosure also provides kits that comprise an binding molecule disclosed herein or an antigen binding portion thereof that can be used to perform the methods described herein. In certain aspects, a kit comprises (i) an antibody, bispecific molecule, immunoconjugate, pharmaceutical composition, nucleic acid (e.g., DNA or mRNA), vector, or cell (e.g., a host cell) disclosed herein, or a combination thereof, and (ii) instructions for use. In some aspects, a kit comprises an antibody, bispecific molecule, immunoconjugate, pharmaceutical composition, nucleic acid (e.g., DNA or mRNA), vector, or cell (e.g., a host cell) disclosed herein, or a combination thereof, in one or more containers.

In some aspects, the kits contain all of the components necessary and/or sufficient to perform a detection assay, including all controls, directions for performing assays, and any necessary software for analysis and presentation of results.

One skilled in the art will readily recognize that the antibodies, bispecific molecules (e.g., bispecific antibodies), immunoconjugates, pharmaceutical compositions, nucleic acids (e.g., DNA or mRNA), vectors, or cells (e.g., a host cell) disclosed herein, or combinations thereof can be readily incorporated into one of the established kit formats which are well known in the art.

### EXAMPLES

### Example 1

### Generation of Antibodies that Preferentially Bind to FIXa Over FIX

Design of Antibody Selections and Antibody Production: A series of human antibodies against human activated FIX was selected from a human antibody Adimab yeast library (ADIMAB, 7 Lucent Drive, Lebanon, N.H. 03766). Antibody selections were performed using three different Factor IX variants:

(i) “non-activatable FIX” (also abbreviated as FIXn) which is Factor IX carrying an arginine to alanine mutation at position 180 (mature numbering) preventing its activation and maintaining Factor IX in the zymogen form (FIXz);
(ii) “free FIXa,” which is a form of activated FIX and is believed to be in a conformation of activated FIX when not bound FVIIIa in a tenase complex); and
(iii) “FIXa-SM,” which is a form of activated FIX with a substrate mimic (e.g., L-Glu-Gly-Arg chloromethyl ketone, i.e., EGR-CMK) covalently bound to the active site, which is intended to mimic the most active conformation of activated FIX.

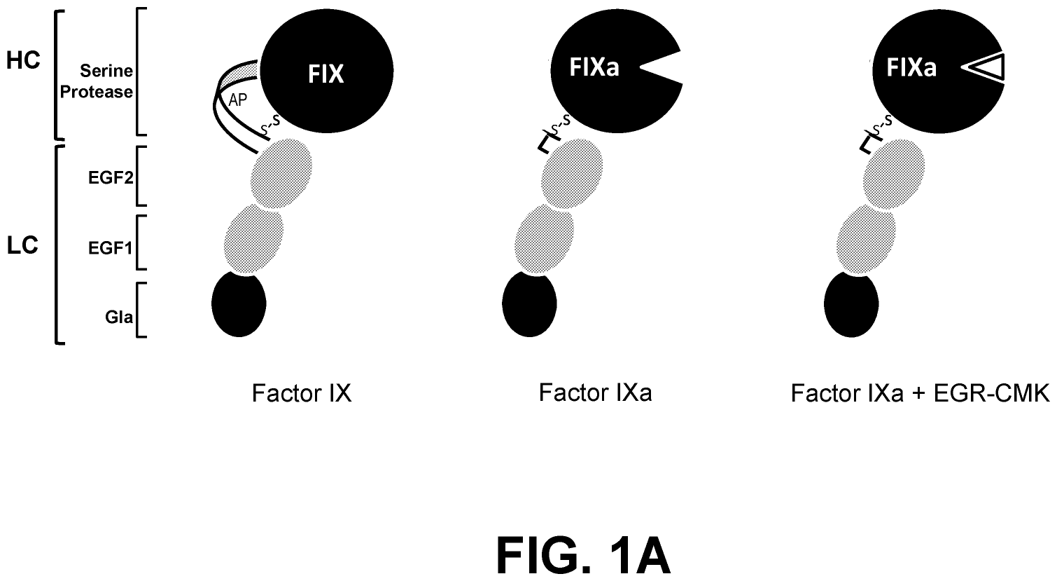

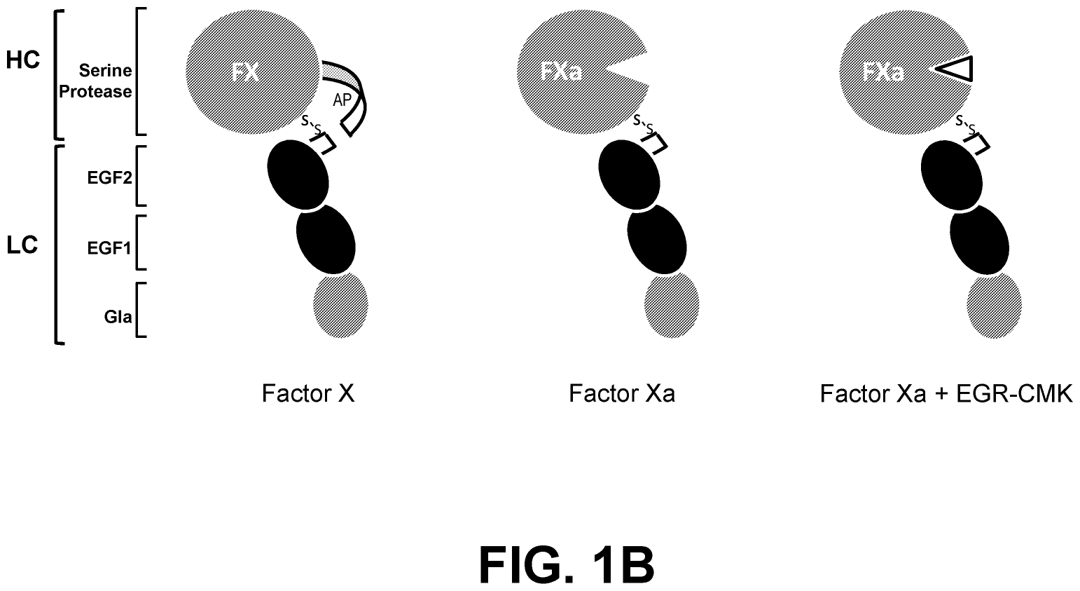

The schematic diagrams of FIX zymogen (e.g., non-activatable FIX), free activated FIX, and FIXa-SM (e.g, Factor IXa+EGR-CMK) are shown in FIG. 1A.

In each case, the FIX sequence was followed by a GS-linker and biotinylation tag at the C-terminus of the molecules. When co-expressed with the biotin ligase, BirA, in the presence of biotin, the resulting FIX molecule will carry a single biotin label to enable selections with the Adimab display library.

Non-activatable FIX was recombinantly expressed with BirA in the presence of biotin and purified according to methods known in the art. Free FIXa, also co-expressed with BirA in the presence of biotin, was generated from the expression of a non-activated precursor (FIX zymogen), which was purified in the same manner as non-activatable FIX. Non-activated FIX precursor was then activated by the addition of recombinant coagulation Factor XIa at a molar ratio of 1:500 in the presence of calcium and subsequently purified by size exclusion chromatography according to methods known in the art.

Addition of a tripeptide chloromethylketone (i.e., EGR-CMK) to free FIXa results in the covalent linkage of the peptide, or substrate mimic, and the active site of FIXa. While the irreversible linkage with the peptide neutralizes FIXa activity, the complex is thought to mimic the substrate-bound, or most active conformation of FIXa. To generate activated Factor IX with a substrate mimic covalently bound to the active site (i.e., FIXa-SM), free FIXa was incubated with a 5-fold molar excess of the peptide in the presence of calcium and 30% ethylene glycol for six hours. Excess peptide was removed either by size exclusion chromatography or dialysis and saturation of the active site was confirmed by mass spectrometry.

The FIX proteins used in the example described above were recombinantly produced according to the methods known in the art. The sequences of the clotting factors are: non-activatable FIX (SEQ ID NO: 773), free FIXa (47 to 191 of SEQ ID NO: 764 and amino acids 227 to 461 of SEQ ID NO: 764), and FIXa-SM are (47 to 191 of SEQ ID NO: 764 and amino acids 227 to 461 of SEQ ID NO: 764 with EGR-CMK covalently bound to the active site) are shown in TABLE 4.

All FIX proteins were buffer exchanged into Tris-buffered saline with 5 mM CaCl₂ as an additive, according to methods known in the art.

A set of antibodies against FIXa were generated, which are capable of preferentially associating with FIXa versus FIX, in a manner similar to that of FVIIIa, are described. To generate these classes of antibodies, Adimab expression libraries were screened in accordance with the methods disclosed in U.S. Publication Nos. 2010/0056386 and 2009/0181855, which are herein incorporated by reference in their entireties. See, e.g., Van Deventer and Wittrup (2014) Methods Mol. Biol. 1319:3-36; Chao et al. (2006), Nature Protocols 1(2):755-768; Feldhaus et al. (2003) Nature Biotechnology 21(2):163-170; Boder and Wittrup (1997) Nature Biotechnology 15(6):553-557, all of which are herein incorporated by reference in their entireties.

Following several iterative rounds of positive selective pressure towards the targeted antigen free FIXa and/or FIXa-SM and negative selective pressure against the non-activatable FIX, colonies were sequenced to identify unique clones, using techniques known in the art. Following antibody selection, 658 antibodies of these selective antibodies were expressed and purified on protein A resin from yeast, according to standard methodology. Subsequently, the 658 antibodies were screened again for their preferential binding to free FIXa and/or mimic-bound FIXa versus non-activatable FIX, this time by Bio-Layer Interferometry (BLI).

Unless otherwise noted, all the antigens in the BLI experiments disclosed herein were purchased from Haematologic Technologies, Inc. (HTI, 57 River Road, Essex Junction, Vt., USA): FIXz (Cat. No. HCIX-0040), free FIXa (Cat. No. HCIXA-0050) and FIXa-SM (Cat. No. HCIXA-EGR). The OctetRed94, Ocetet QK 384, or the Octet HTX system were used for all BLI experiments. All methods, with regard to antibody and antigen concentrations as well as incubation times for the various steps, are based on manufacturer recommendations. Baseline steps were 60 seconds. Antibodies were loaded onto anti-human IgG Quantitation probes at concentrations ranging from 100-200 nM or 10-15 ug/mL for 180 seconds. Antigen concentrations ranged from 10-250 nM and the association of the antigen to the antibody-loaded probes range from 90-180 seconds. The dissociation steps were performed in buffer alone for 90-180 seconds. All BLI experiments were performed with buffers including 100-200 nM NaCl, 25-50 mM Tris pH 7.4-8.0 or 25-50 mM Hepes pH 7.4-8.0, with or without 0.1% BSA, with or without 2.5-5 mM CaCl₂). Additional details for specific experiments are given in the examples below. The relative binding of various antibodies to a particular antigen is generally reported as the “response,” or the change in nanometer shift from the beginning to the end of the association phase. The antigen binding response is dependent on the amount of antibody loaded onto the probe during the loading phase. Therefore, if the amount of antibody loaded onto the probe is the same, and the concentration of the antigens and the length of the association phase are the same, these responses are comparable. In other cases, the ForteBio Data Analysis software may be used to calculate a K_{D}. In all cases, the lack of non-specific binding of the antigens to the anti-human IgG AHQ probes was confirmed.

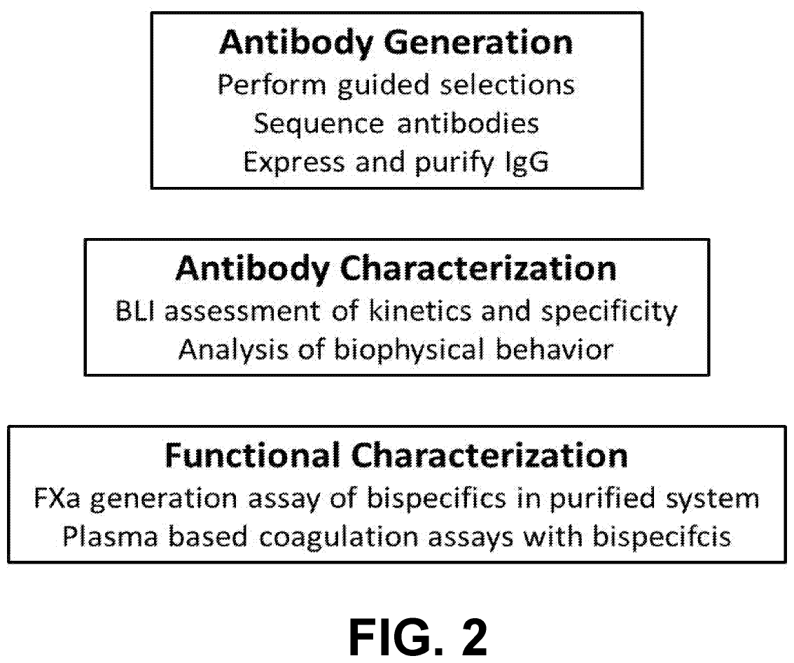

An outline of the experimental triage to identify antibodies that preferentially bound free FIXa and FIXa-SM is shown in FIG. 2. Details of the triage scheme are provided in the “Characterization of Antibody Binding to Target Antigen” and “Screening of Antibodies for Biophysical Behavior” sections below.

This additional antibody triage resulted in a set of 93 antibodies that may preferential bind to free FIXa and/or FIXa-SM. The amino acid and nucleic acid sequences of the variable regions are provided below. The germline and CDR sequences of the VH and VL of those 93 antibodies are shown in FIGS. 3A, 3B, 3C and FIG. 3D (except BIIB-9-1335 and BIIB-9-1336, which were obtained by the method shown in Example 5 below).

Characterization of Antibody Binding to Target Antigen FIXa: To identify antibodies discovered in our preliminary selections that demonstrated preferential binding to free FIXa in comparison to non-activatable FIX, the 658 antibodies purified from the yeast Adimab library were screened using BLI in a monovalent assay format with 150 nM antibody using anti-Human IgG Quantitation (AHQ) dip and read biosensors (Pall ForteBio: Catalog #18-5005). BLI was performed on the OctetRed94 or the Octet HTX system, according to standard procedure. Briefly, biosensors were equilibrated in Tris-buffered saline supplemented with 5 mM CaCl₂ and 0.1% bovine serum albumin (TBSFCa). Plates were then transferred to the instrument. A soak step was performed on the instrument, with incubation of the biosensors for 60 seconds in TBSFCa. Then, the biosensors were transferred to wells containing the representative IgG (150 nM in TBSFCa) for 180 seconds. Following this, the biosensors were incubated in TBSFCa for 60 seconds to establish baseline. Next, the biosensors were transferred to wells containing the desired antigen (100 nM in TBSFCa) and incubated for 90 seconds. Finally, the sensors were transferred to TBSFCa alone for 90 seconds incubation.

Nearly all of the 658 antibodies identified in the initial yeast library selections directed towards free FIXa specificity displayed greater approximate binding affinity to free FIXa than to non-activatable FIX. Four antibodies (i.e., BIIB-9-397, BIIB-9-578, BIIB-9-612, and BIIB-9-631) had greater binding affinity to non-activatable FIX than to free FIXa in the BLI assay.

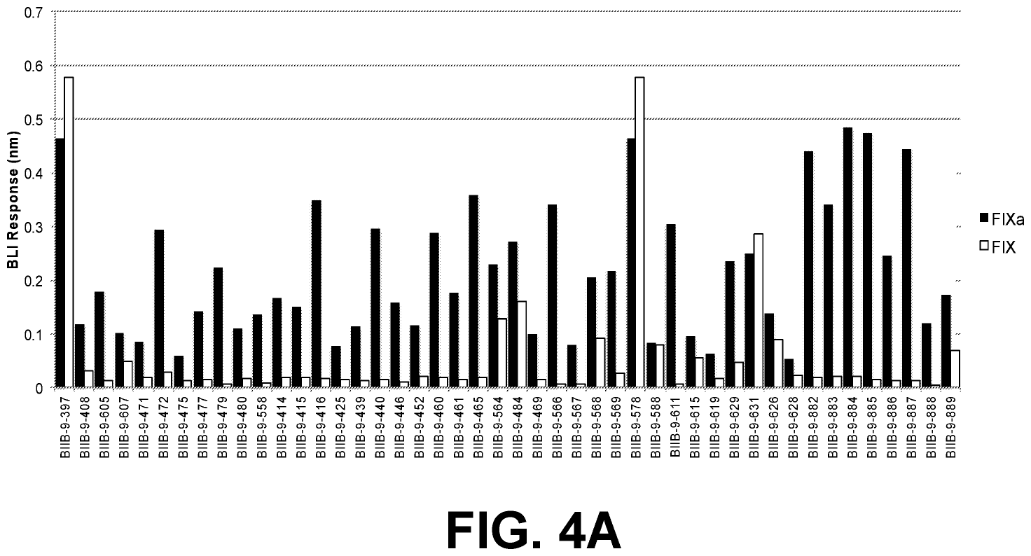

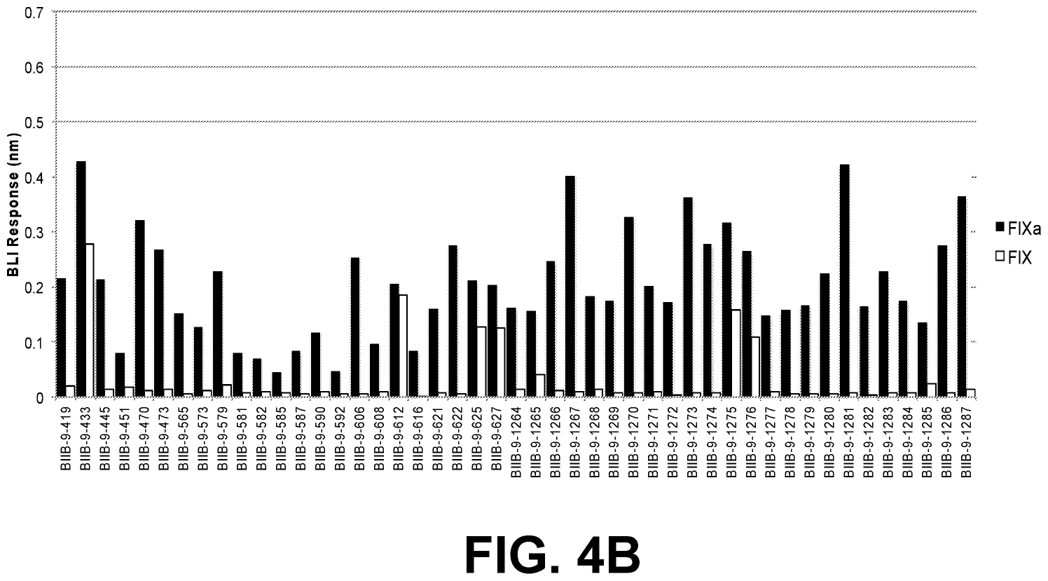

The maximum response value (nm) following the antigen association phase, for either 100 nM free FIXa or non-activatable FIX, was plotted for each of the 93 antibodies identified in the selections (FIG. 4A and FIG. 4B).

Antibodies whose binding affinity to free FIXa, as measured by BLI, was higher than to non-activatable FIX are likely to preferentially bind free FIXa, e.g., due to the selective binding to an epitope on human FIXa which is unique to FIXa, or to an epitope on FIXa which is significantly different from a corresponding epitope on FIX zymogen. In other words, the preferential binding of the antibodies to FIXa may be driven, e.g., by the binding to an epitope that is absent in FIX zymogen, or by the higher binding affinity to the same epitope or a variant thereof (e.g., an overlapping epitope or a conformationally different epitope).

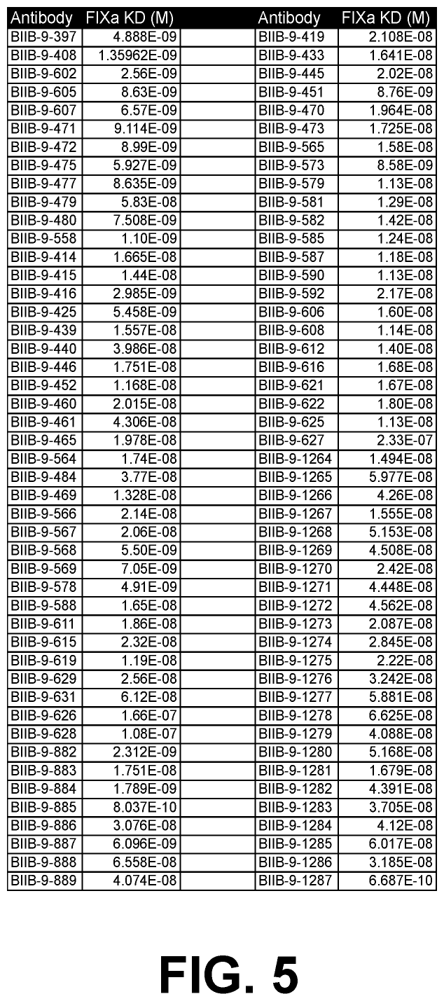

FIG. 5 shows a table of apparent monovalent affinity (K_{D}) to free FIXa for each of the listed antibodies as determined by 1:1 fitting algorithms provided in the ForteBio Data Analysis 9.0 software.

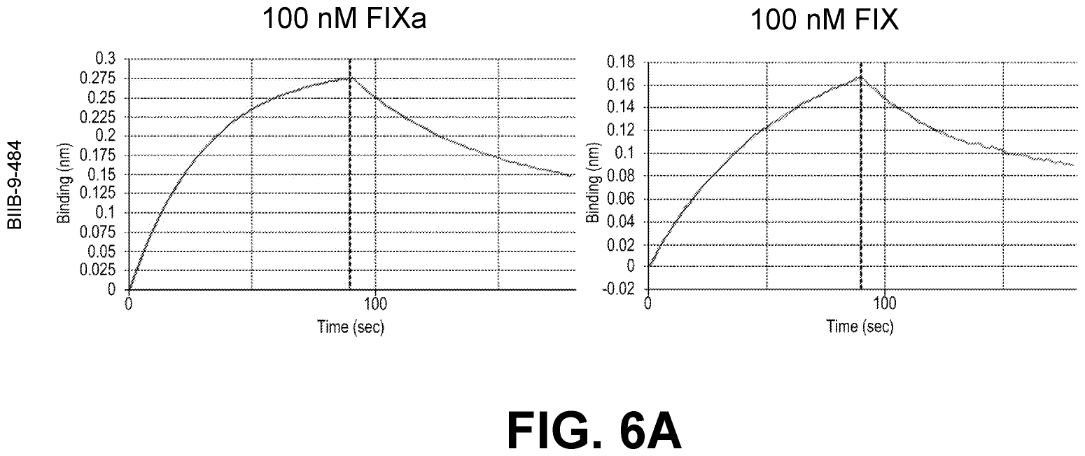

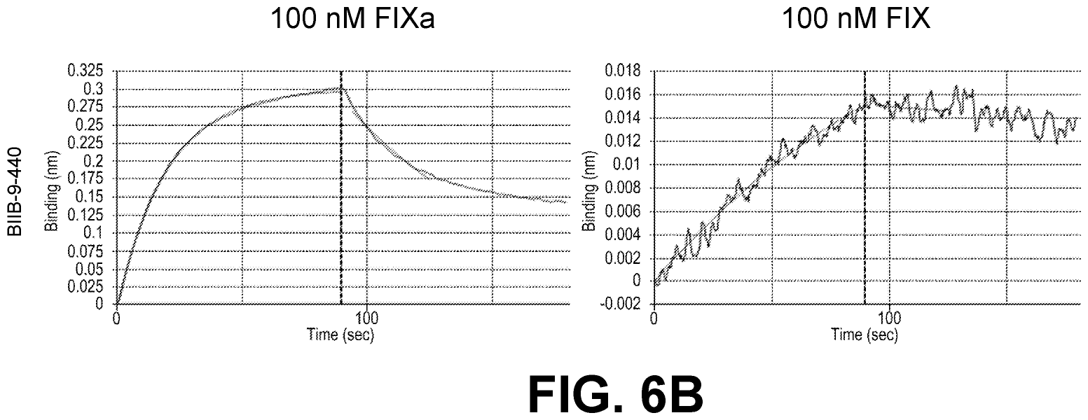

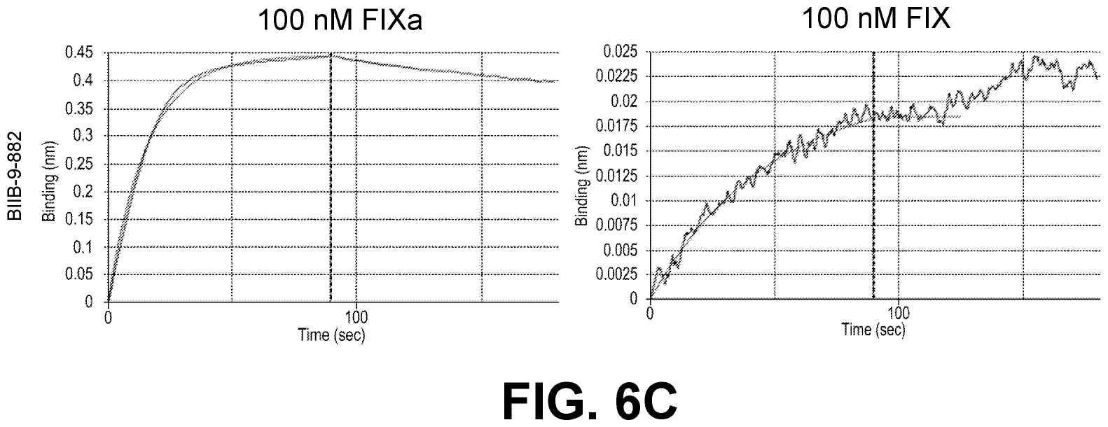

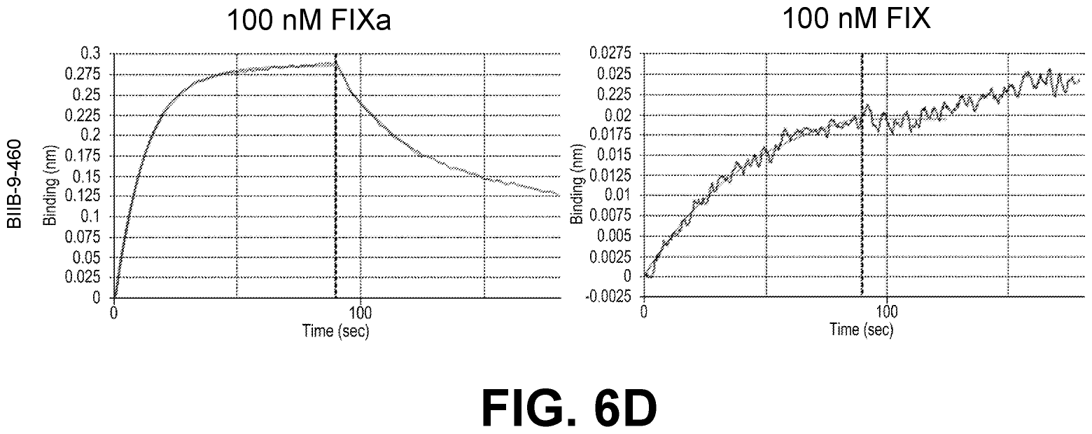

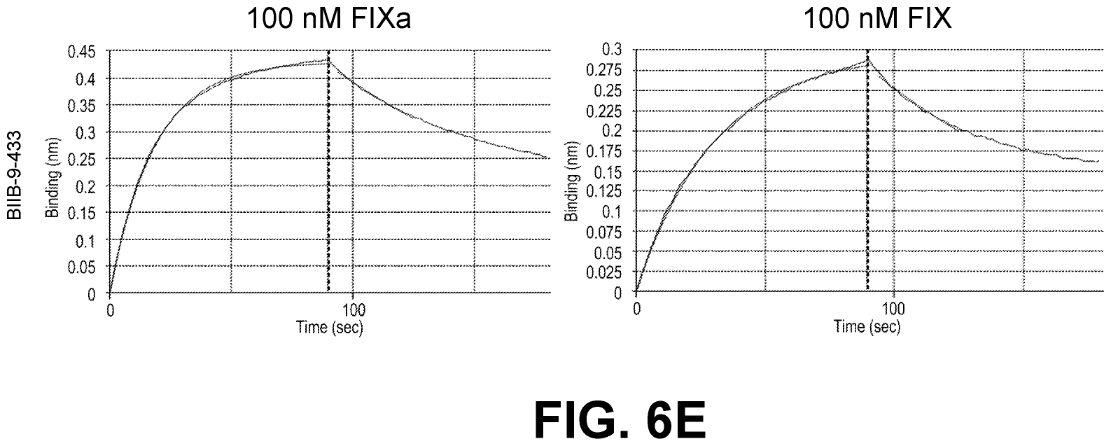

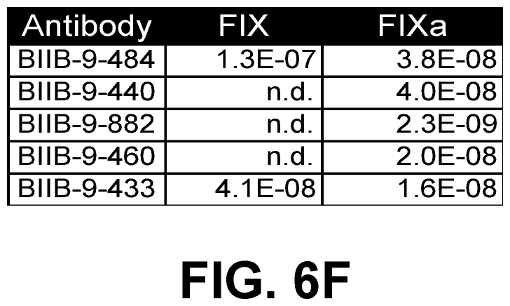

BLI binding profiles for a select subset of antibodies analyzed are provided in FIGS. 6A-6E. Approximate monovalent affinities to free FIXa or non-activatable FIX for a select set of antibodies test, as determined by BLI, are listed in FIG. 6F.

It is predicted that bispecific antibodies that preferentially recognize the more active conformation of FIXa (i.e., FXa-SM), will have higher activity than those that preferentially bind to free FIXa.

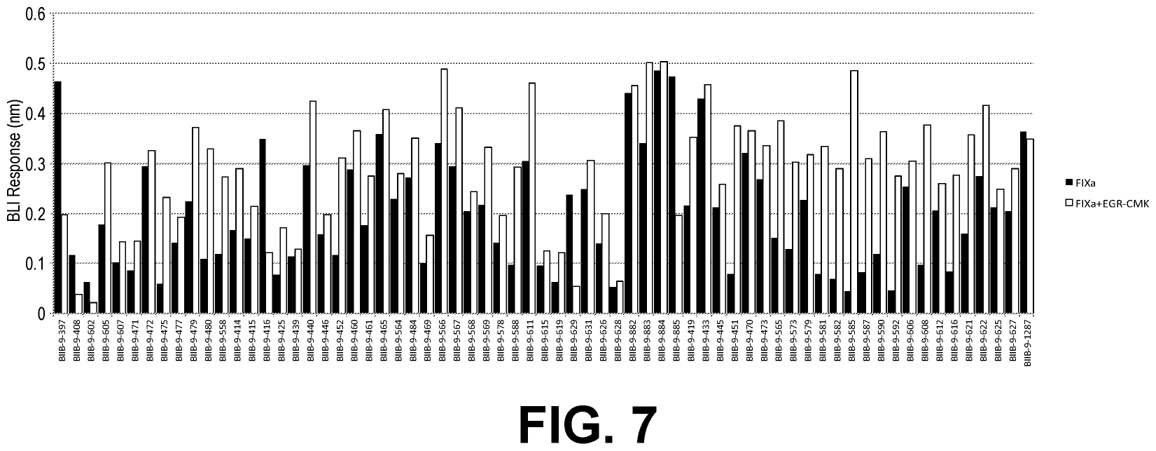

As demonstrated in FIG. 7, a subset of the 93 antibodies that displayed greater binding affinity to free FIXa than to non-activatable FIX were further assessed for their binding to 100 nM free FIXa and to 100 nM FIXa-SM in a BLI monovalent binding assay with 150 nM antibody on the OctetRed94 or the Octet HTX system according to manufacturer's procedures. Briefly, anti-Human IgG Quantitation (AHQ) dip and read biosensors (Pall Fortebio: Catalog #18-5005) were equilibrated in TBSFCa. Plates were then transferred to the instrument. A soak step was performed on the instrument, with incubation of the biosensors for 60 seconds in TBSFCa. Then, the biosensors were transferred to wells containing the representative IgG (150 nM in TBSFCa) for 180 seconds. Following this, the biosensors were incubated in TBSFCa for 60 seconds to establish baseline. Then, the biosensors were transferred to wells containing the desired antigen (100 nM in TBSFCa) and incubated for 90 seconds. Finally, the sensors were transferred to TBSFCa alone for a 90-second incubation.

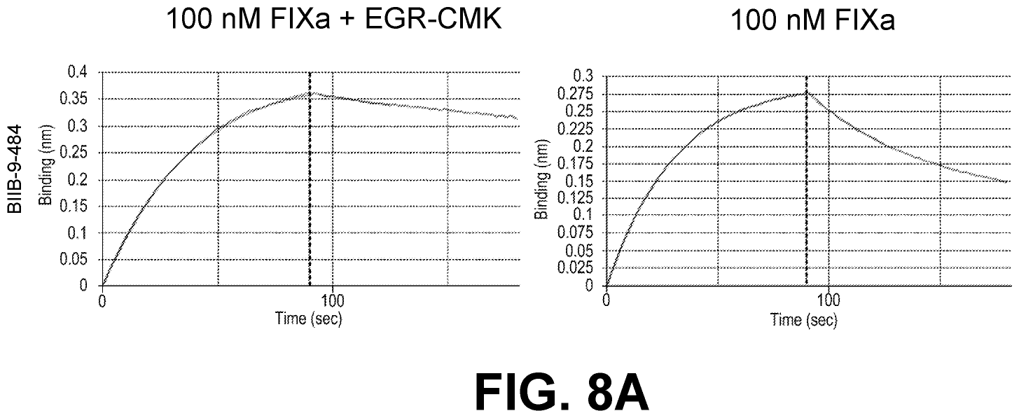

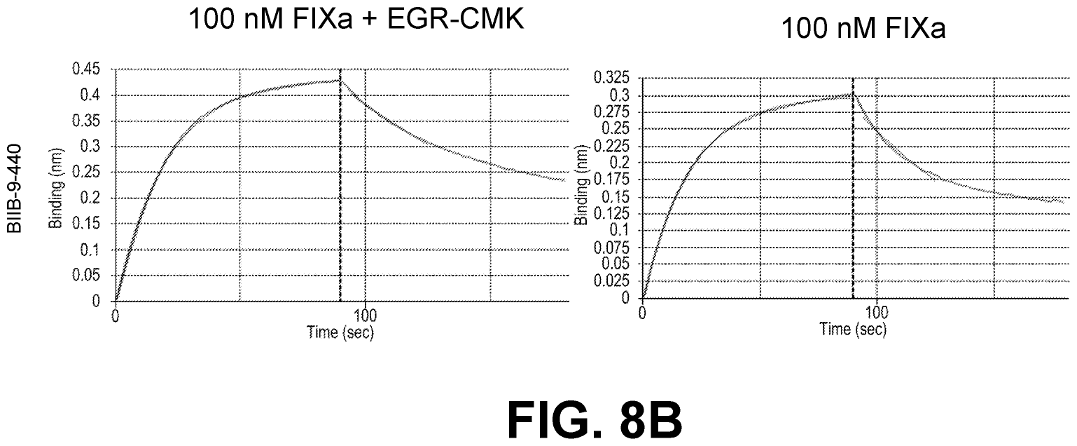

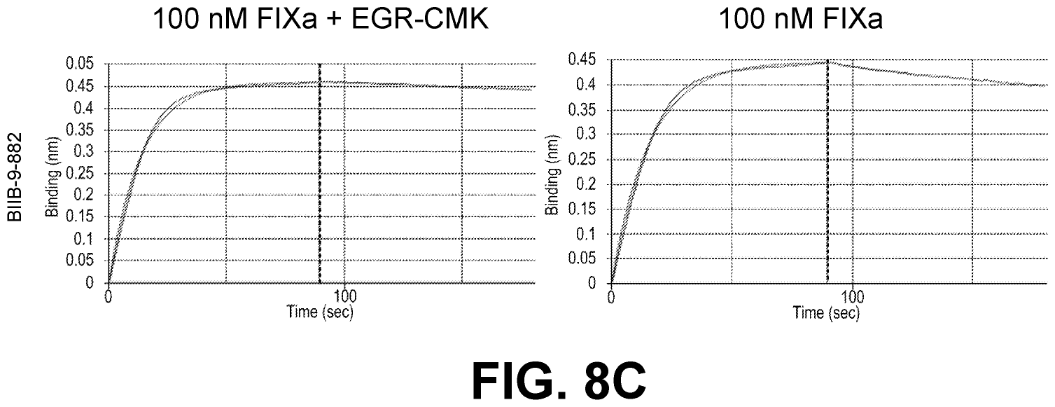

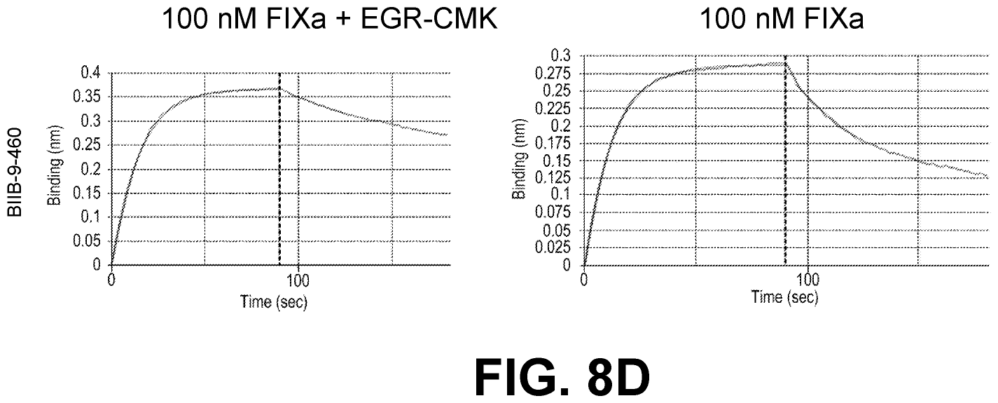

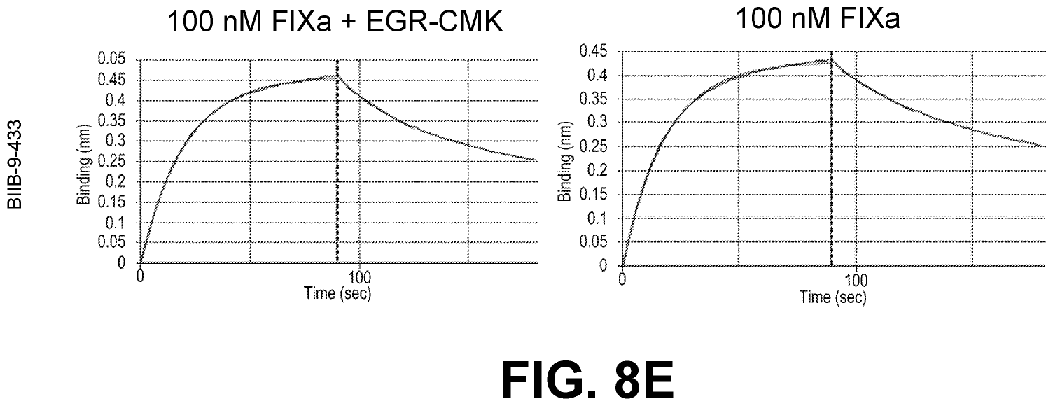

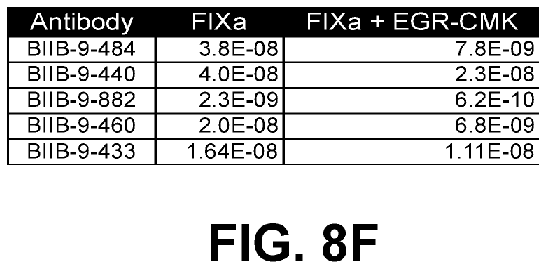

For a number of antibodies, greater binding to FIXa-SM than to free FIXa was observed. Representative examples of BLI binding profiles for association to each target antigen are provided in FIG. 8A-8E. A table in FIG. 8F, lists the apparent affinities for tested antibodies to each target antigen according to fitting algorithms provided in the ForteBio Data Analysis 9.0 software.

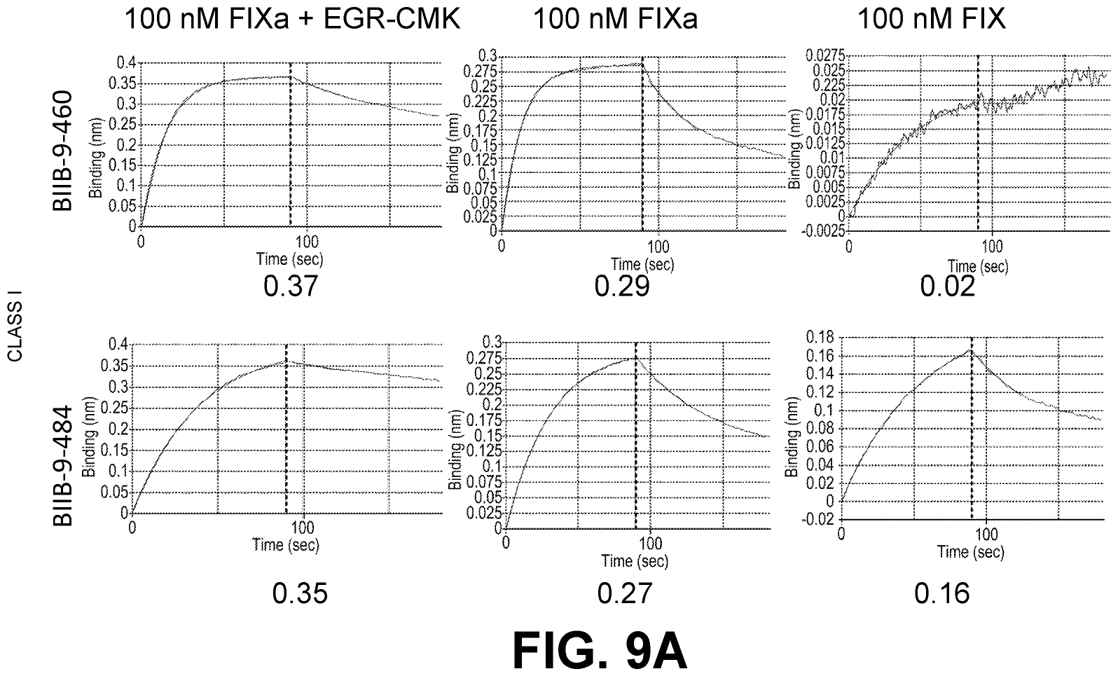

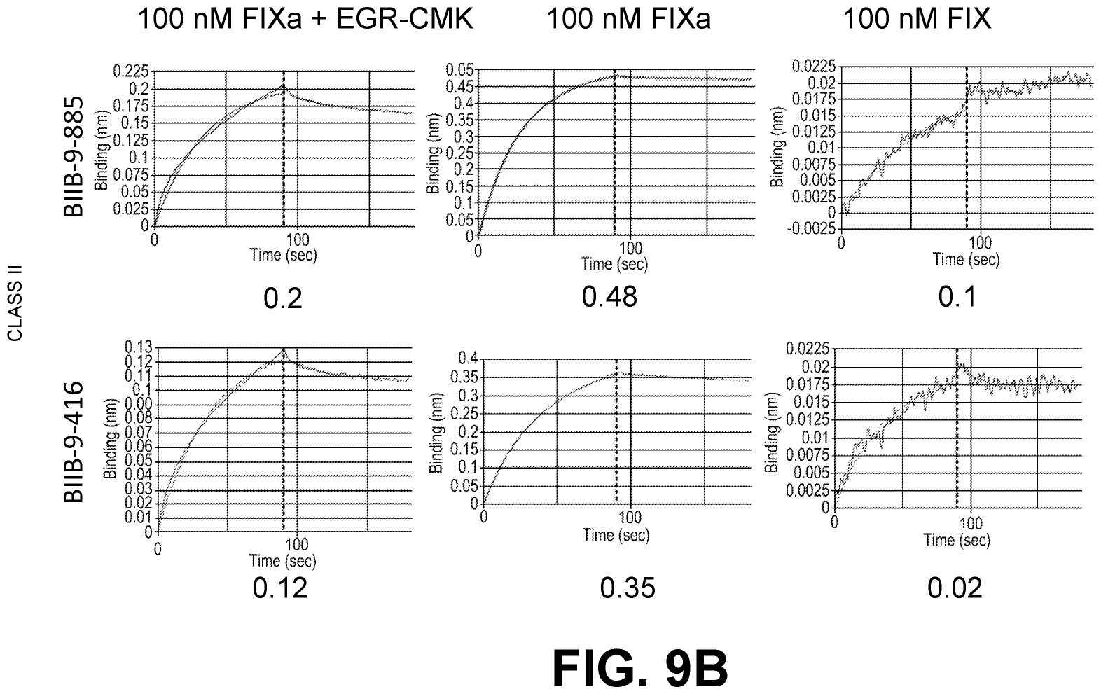

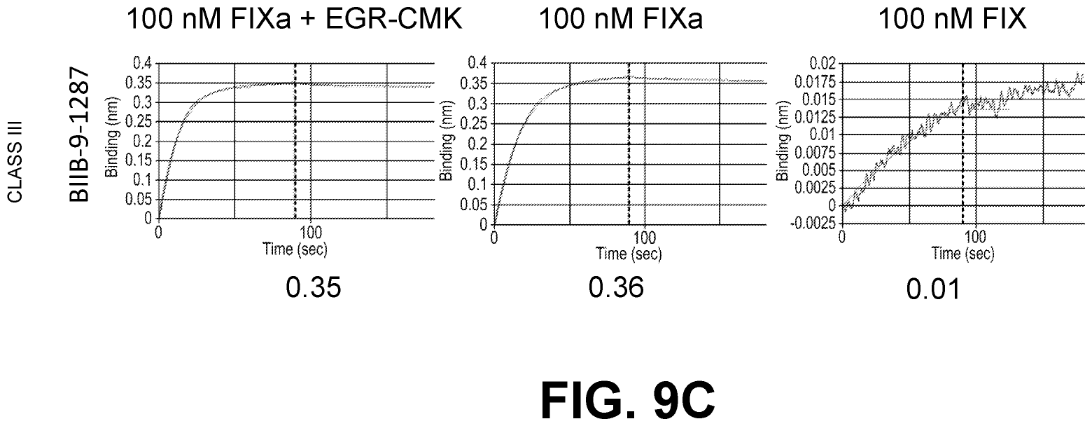

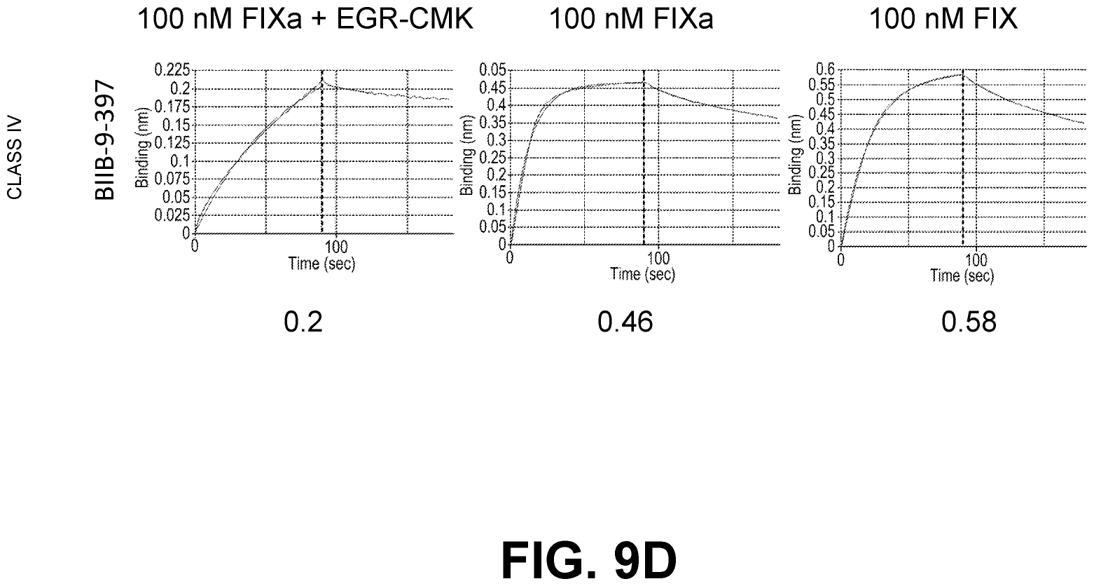

Four classes of antibodies were identified from BLI experiments intended to address specificity of the antibodies discovered in the selections: Class I: Antibodies preferentially bind to FIXa-SM compared to either free FIXa or non-activatable FIX. BIIB-9-460 and BIIB-9-484 are examples of this class, as shown in FIG. 9A; Class II: Antibodies preferentially bind to free FIXa than to FIXa-SM or non-activatable FIX, as represented by BIIB-9-885 and BIIB-9-416 (FIG. 9B); Class III: Antibodies bind to either FIXa-SM or free FIXa with near equivalency, but do not appreciably associate with non-activatable FIX. BIIB-9-1287 serves as an example of this class (FIG. 9C); and, Class IV: Antibodies preferentially bind to non-activatable FIX compared to free FIXa or FIXa-SM (FIG. 9D). BIIB-9-397 serves as an example of this class

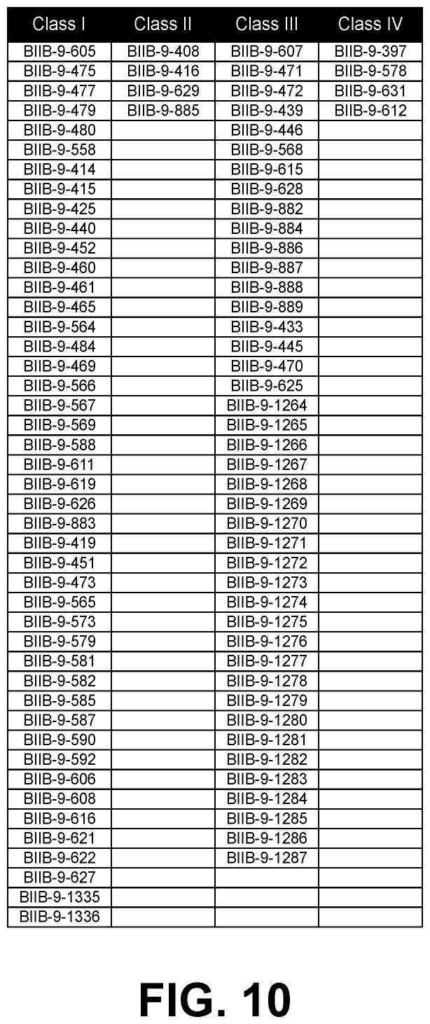

A table listing the class assignment for all of the anti-FIX antibodies (e.g., anti-FIXa antibody or anti-FIXz antibody) disclosed herein is provided in FIG. 10.

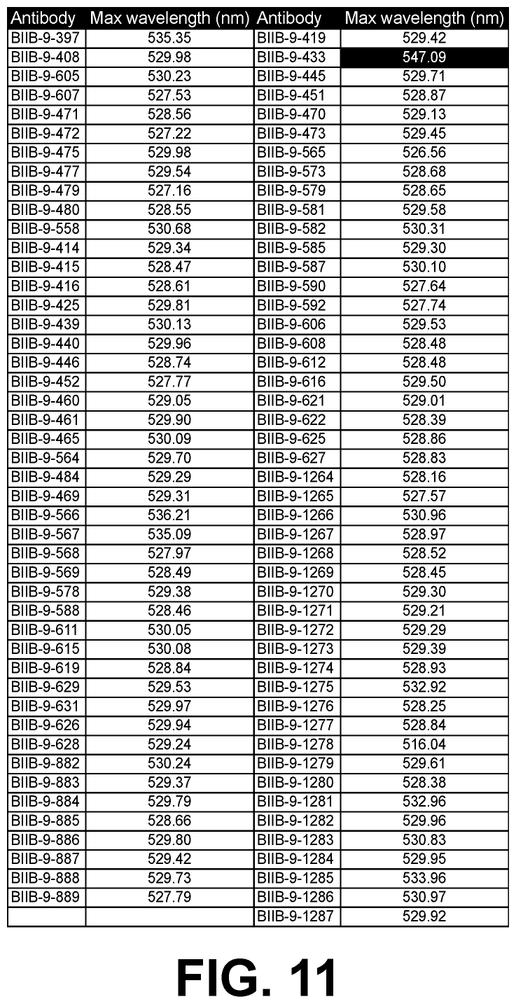

Screening of Antibodies for Biophysical Behavior: 93 anti-FIX antibodies were examined by affinity capture self-interaction nanoparticle spectroscopy (AC-SINS), an assay that micro-concentrates target antibody on the surface of nanoparticles to assess self-association. AC-SINS screens to identify antibodies with poor biophysical properties, e.g., self-aggregation, in relation to their peers were performed according to methods described in the literature. See, e.g., Liu et al. (2014) MAbs. 6(2):483-92; and Wu et al. (2015) Protein Engineering, Design and Selection 28: 403-414, both of which are herein incorporated by reference in their entireties. Each antibody was captured on the surface of gold nanoparticles with a starting concentration of 40 μg/ml in a volume of 100 μl and incubated at room temperature for two hours. Following the incubation, absorbance across a spectrum of wavelengths was determined. In cases of antibody self-association, the inter-particle distance decreases, resulting in higher wavelengths of maximum absorbance. In comparison to internal standards of biophysical behavior, maximum wavelengths of greater than 540 nm are indicative of antibodies that have a propensity to self-interact. A table listing the calculated maximum wavelengths for 93 anti-FIX antibodies as determined by AC-SINS is shown in FIG. 11.

### Example 2

### Generation of Antibodies that Preferentially Bind to FX Over FXa

Design of Antibody Selections and Antibody Production: With the goal of obtaining antibodies that selectively bind to FX zymogen (e.g., non-activatable factor X (FX)) over activated Factor X (FXa), antibody selections from an yeast Adimab human antibody library were performed using three different Factor X variants:

(i) “non-activatable Factor X” (“FXn”), which is FX carrying an arginine to alanine mutation at position 194 (mature numbering) preventing its activation and maintaining FX in the zymogen form. This was the FXz (zymogen FX) that was used for BLI experiments. FXn was produced and biotinylated in house as described below;
(ii) “free FX,” (“FXa”) which is a form of activated FX without any substrate mimic and is believed to be in a wild-type activated FX conformation (HTI, Cat No. HCXA-0060); and,
(iii) “FXa-SM,” which is a form of activated Factor X with a substrate mimic (e.g., EGR-CMK) covalently bound to the active site (HTI, Cat No. HCXA-EGR). In some cases the substrate mimic was biotinylated (BEGR-CMK) and covalently bound to the active site (HTI, Cat. No. HCXA-BEGR).

The schematic diagrams of FX zymogen, free FXa, and FXa-SM (Factor Xa+EGR-CMK) are shown in FIG. 1B.

The non-activatable FX sequence was followed by a GS-linker and biotinylation tag at the C-terminus of the molecule. When co-expressed with the biotin ligase, BirA, in the presence of biotin, the resulting FX molecule carries a single biotin label to enable selections with the Adimab display library.

Non-activatable FX was recombinantly expressed with BirA in the presence of biotin and purified according to methods known in the art.

As is the case for FIXa, a tripeptide chloromethylketone (i.e., EGR-CKM) of the sequence EGR can covalently modify the active site of FXa, mimicking a substrate-bound conformation of FXa.

For the purposes of negative selections, free FXa (HTI, Cat. No. HCXA-0060) and human FXa-SM (HTI, Catalog No. HCXA-EGR and HCXA-BEGR) were purchased from Haematologic Technologies Incorporated (HTI).

All FX proteins, whether produced in-house or purchased, were buffer exchanged into Tris-buffered saline with 5 mM CaCl₂ as an additive, according to methods known in the art. In this present disclosure, a set of antibodies generated against non-activatable FX, which demonstrate greater binding to non-activatable FX than to FXa (e.g., free FXa or FXa mimic) (in a manner similar to that of FVIIIa), are described.

As in the case of the above-disclosed anti-FIX antibodies, Adimab expression libraries were screened in accordance with the methods disclosed in U.S. Publication Nos. 20100056386 and 20090181855. Following several iterative rounds of positive selective pressure towards the targeted antigen FX, i.e., non-activatable FX, as well as negative selective pressure against the free FXa or FXa-SM colonies were sequenced to identify unique clones using techniques known in the art. Following antibody selection, more than 800 antibodies were expressed and purified on protein A resin from yeast, according to standard methodology. The experimental triage to identify antibodies that selectively bind to non-activatable FX is outlined in FIG. 2.

Details of the second step of the triage scheme are provided in the sections entitled “Characterization of Antibody Binding to Target Antigen Factor X (FX)” and “Screening of Antibodies for Biophysical Behavior,” see below. The second step of antibody triage resulted in a set of 94 antibodies against FXn, which may preferentially bind to FXn compared to active FXa (e.g., wild-type FXa or FXa-SM). The amino acid and nucleic acid sequences of the variable regions are provided above. A table of the germline and the CDR of each of the 94 antibodies is depicted in FIGS. 12A, 12B, and 12C.

Characterization of Antibody Binding to Target Antigen Factor X (FX): To further determine which of the discovered antibodies demonstrate greater binding to FX zymogen than to FXa-SM, the more than 800 antibodies purified from yeast were screened using Bio-Layer Interferometry (BLI) in a monovalent assay format with 200 nM antibody. Non-activatable FX and FXa-SM were obtained from HTI. Free FXa and FXa-SM behaved the same in binding experiments and can be used interchangeably; therefore free FXa results are not shown. Because the data presented for free FXa corresponds to FXa-SM, the observations using FXa-SM are equally applicable to free FXa.

BLI was performed on the OctetRed94 or the Octet HTX system, according to standard procedure. Briefly, anti-Human IgG Quantitation (AHQ) dip and read biosensors (Pall Fortebio: Catalog No. 18-5005) were equilibrated in TBSFCa. Plates were then transferred to the instrument. A soak step was performed on the instrument, with incubation of the biosensors for 60 seconds in TBSFCa. Then, the biosensors were transferred to wells containing the representative IgG (200 nM in TBSFCa) for 180 seconds. Following this, the biosensors were incubated in TBSFCa for 60 seconds to establish baseline. Then, the biosensors were transferred to wells containing the desired antigen (200 nM in TBSFCa), and association occurred for 90 seconds. Finally, the sensors were transferred to TBSFCa alone for a 90-second incubation. The maximum response value (nm) following the association phase, for either 200 nM FXn or FXa-SM, was plotted for each of the 94 antibodies identified in the selections. Nearly all of the antibodies identified in selections directed towards FXn specificity demonstrated greater binding affinity to FX than to covalently modified FXa (FIG. 13).

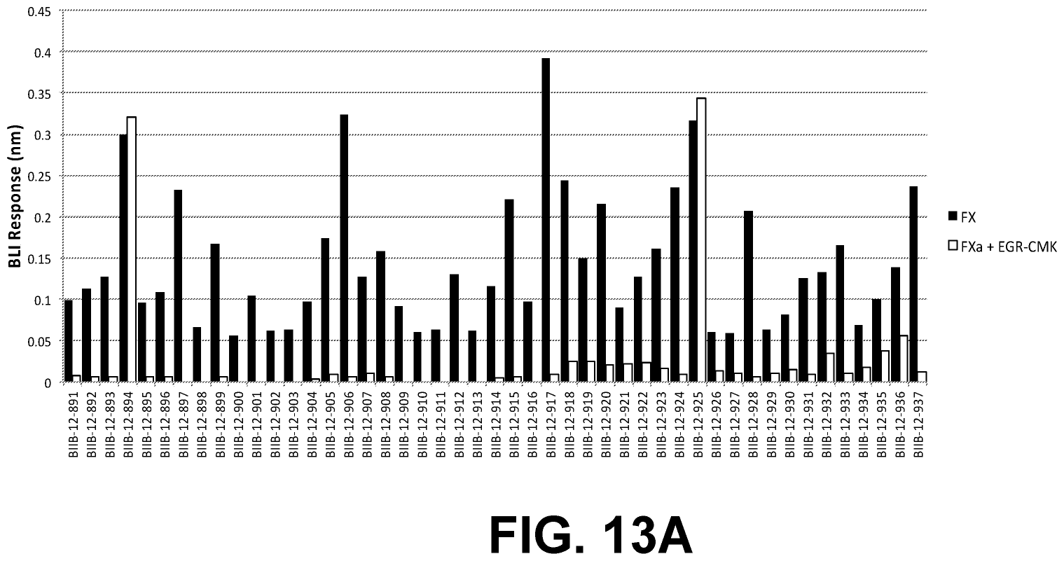

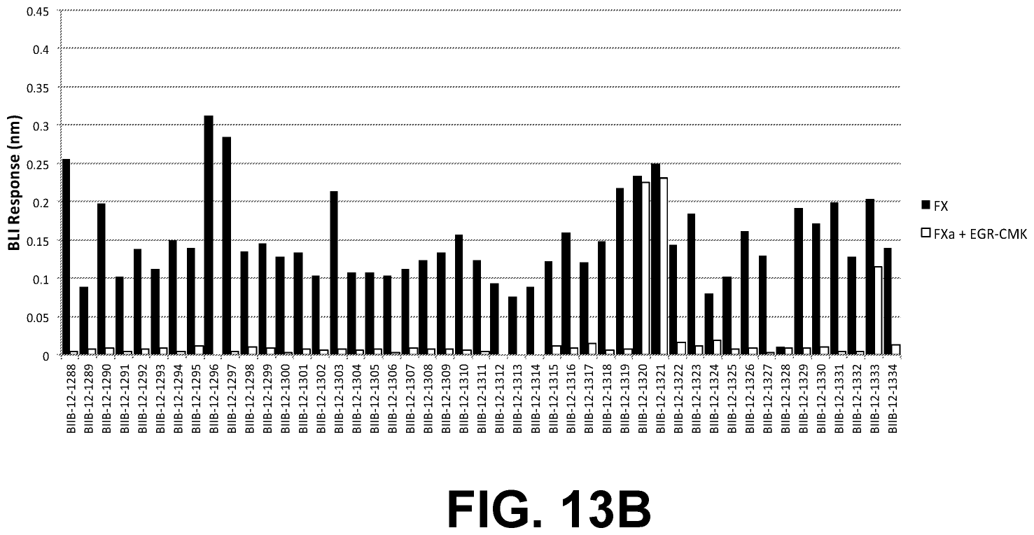

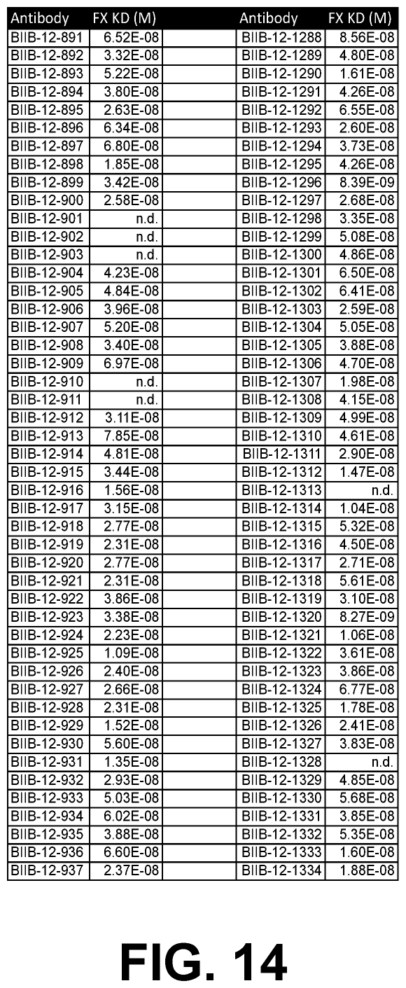

Only BIIB-12-894, BIIB-12-925, BIIB-12-1320, and BIIB-12-1321 displayed greater binding to FXa-SM. It is expected that the antibodies that preferentially bind to FXn compared to activated FX (e.g., free FXa or FXa-SM) preferentially bind to FX zymogen compared to FXa. Therefore, the anti-FIXn antibodies can also be referred to as anti-FXz antibodies. FIG. 14 includes a table listing the apparent affinities for tested antibodies to FXn, according to fitting algorithms provided in the ForteBio Data Analysis 9.0 software.

Screening of Antibodies for Biophysical Behavior: 94 anti-FXz antibodies were examined by affinity capture self-interaction nanoparticle spectroscopy (AC-SINS), as described for the above-disclosed anti-FIXa antibodies. AC-SINS screens to identify antibodies with poor biophysical properties (e.g., propensity to self-associate) in relation to their peers were performed according to methods described in the literature. See, e.g., Liu et al. (2014) MAbs 6(2):483-92. Antibody was captured on the surface of gold nanoparticles with a starting concentration of 40 pg/ml in a volume of 100 μl and incubated at room temperature for two hours. Following the incubation, the absorbance across a spectrum of wavelengths was determined. In cases of antibody self-association, the inter-particle distance decreases, resulting in higher wavelengths of maximum absorbance. In comparison to internal standards of biophysical behavior, maximum wavelengths of greater than 540 nm are indicative of antibodies that have a propensity to self-interact. FIG. 15 includes a table listing the calculated maximum wavelengths for 94 anti-FXz antibodies, as determined by AC-SINS.

### Example 3

### Construction of Bispecific Antibodies and Evaluation of FVIIIa-Like Activity in a Chromogenic Assay

Bispecific antibodies consisting of two different Fab arms, one of which preferentially binds to activated FIX (“FXa”) over FIX zymogen (Classes I, II, and III antibodies) and the second of which preferentially binds to FX zymogen over FXa (Class V antibodies) were generated from the individual antibodies described in Examples 1 and 2. Additional bispecific antibodies consisting of a first Fab arm that preferentially binds to FIX zymogen over FIXa (Class IV antibodies) and a second Fab arm that preferentially binds to FXz over FXa (Class V antibodies) or that preferentially binds to FXa over FXz (Class VI antibodies) were generated. Other bispecific antibodies consisting of a first Fab arm that preferentially binds to FIXa over FIXz (Classes I, II, and III antibodies) and a second Fab arm that preferentially binds to FXa over FXz (Class VI antibodies) were also generated.

The disclosed bispecific antibodies can be generated using methods known in the art without undue experimentation. See, e.g., Kontermann & Brinkmann (2015) Drug Discovery Today 20:838-847 and references cited therein; Spies et al. (2015) Molecular Immunology 67:95-106 and references cited therein; Byrne et al. (2013) Trends in Biotechnology 31:621-632 and references cited therein; Strop et al. (2012) J. Mol. Biol. 420:204-219 and references cited therein; all of which are herein incorporated by reference in their entireties. See also, which are herein incorporated by reference in their entireties.

The bispecific antibodies either consisted of two different heavy chains and two different light chains, or two different heavy chains and a common light chain in an IgG1-like format. In an additional subset, the bispecific antibodies were of the IgG4 subclass. These bispecific antibodies were screened for their ability to replace the activity of FVIIIa (i.e., ability to mimic FVIIIa), first in a chromogenic assay and second in a plasma-based coagulation assay.

Assay buffer, phospholipids, CaCl₂, and FXa chromogenic substrate S-2765 were purchased from Diapharma as part of the Chomogenix Coatest SP Factor VIII kit (Cat. No. K824086). All proteins were diluted using a 1X preparation of the assay buffer. Each bispecific antibody (25 μL) was mixed with 20 μL of FX (HTI, Cat. No. HCX-0050) diluted to 750 nM, 20 μL of FIXa (HTI, Cat. No. HCIXA-0050) diluted to 75 nM, and 10 μL of phospholipids at room temperature. After 5 minutes, 25 μL of CaCl₂ was added.

After an additional 10 minutes, 50 μL of substrate S-2765 was added and the absorbance at 405 nM was read in a Biotek Synergy2 plate reader every 15 seconds for one hour. Initial rates were determined by the change in OD 405 nM over time from the linear portion of each absorbance curve. In the absence of FVIIIa, hFIXa is a poor enzyme that only generates a small amount of FXa, leading to a basal level of FXa substrate cleavage as measured by the change in OD at 405 nm over time (mOD/minute). An increase in the rate of FXa substrate cleavage upon addition of the bispecific antibody indicates its ability to facilitate the activation of FX by FIXa (thereby replacing FVIIIa-like function) in a FXa generation assay. Using this method, 202 antibodies were identified that mimicked FVIIIa activity, i.e., have a rate of FXa substrate cleavage at least three standard deviations above the mean basal rate in the absence of the added bispecific antibody (5.88 mOD/minute).

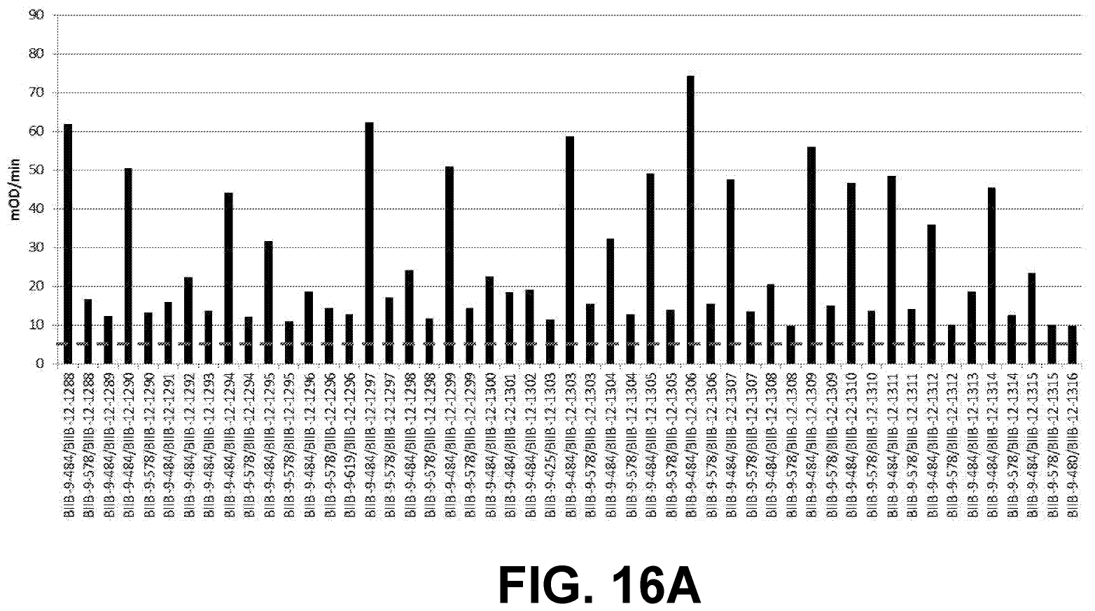

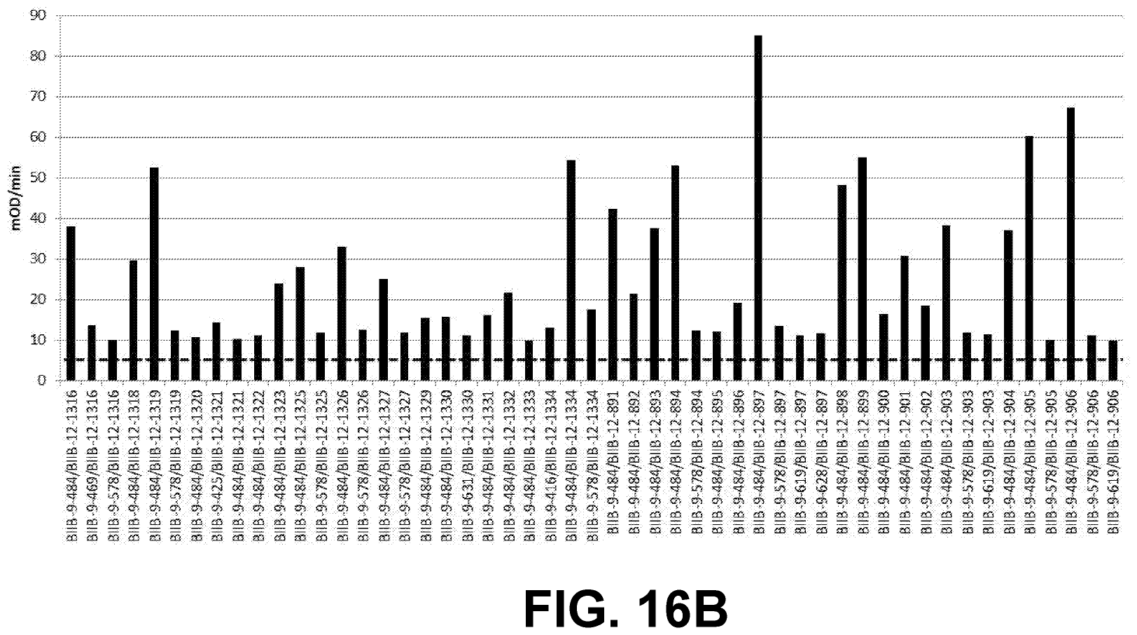

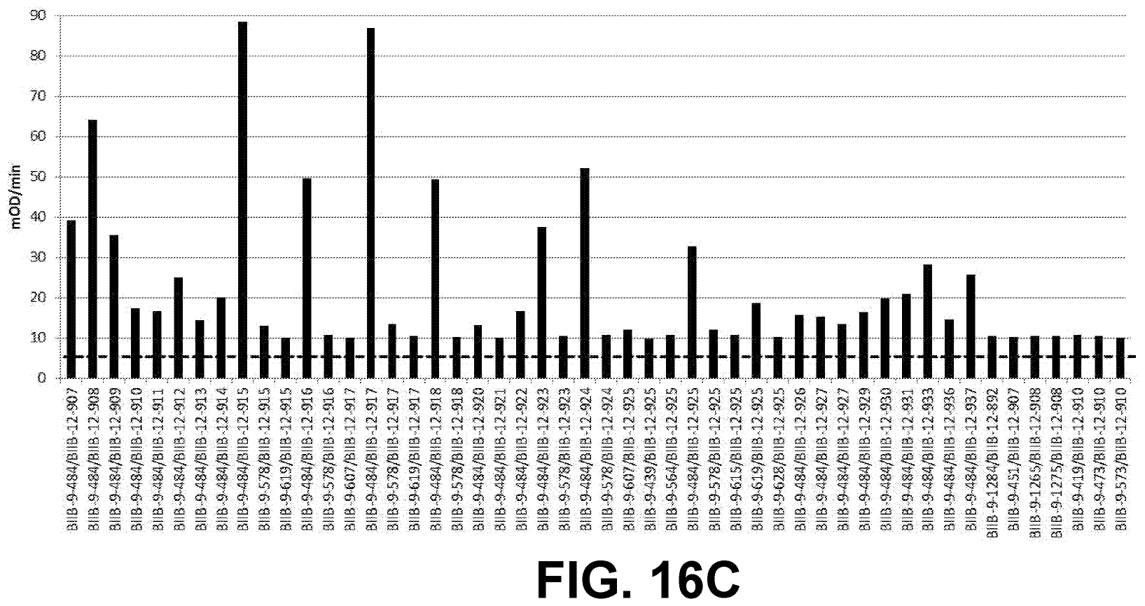

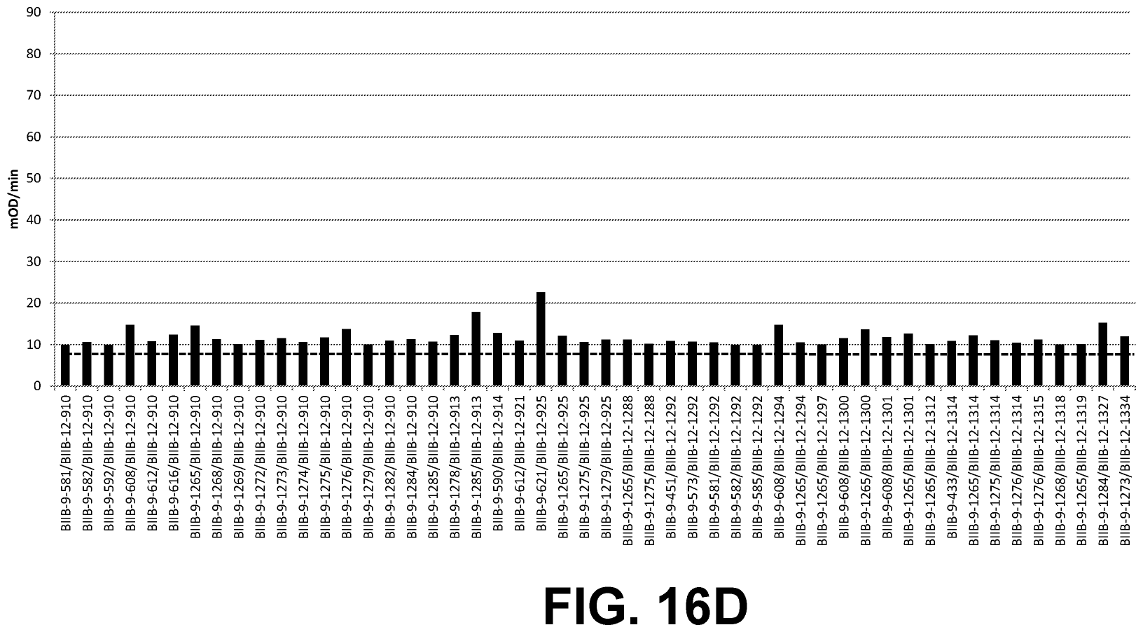

The change in OD over time in the absence of bispecific antibody (baseline), as well as the change in OD over time for 3 different bispecific antibodies: BIIB-9-484/BIIB-12-915, BIIB-9-619/BIIB-12-925 and BIIB-9-578/BIIB-12-917, which are capable of replacing FVIIIa-like function, is shown in FIGS. 16A-16D.

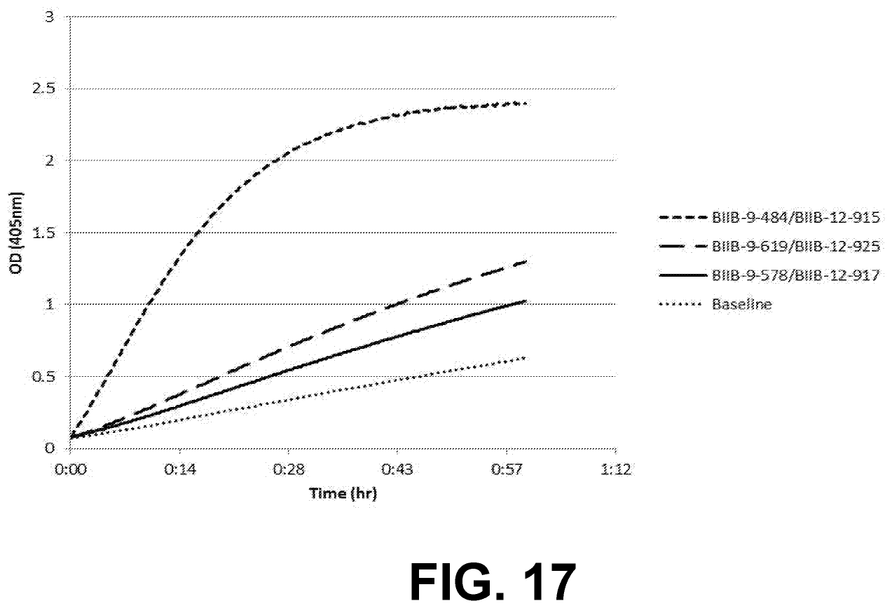

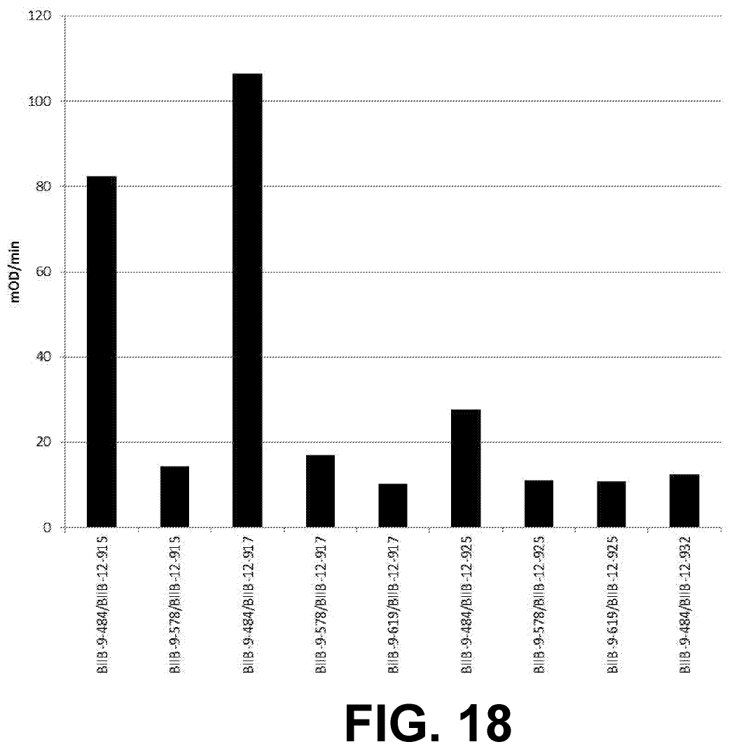

Rates for all of the 202 IgG1 bispecific antibodies capable of replacing FVIIIa-like function are shown in FIG. 16A-16D. Rates for a subset of these antibodies in the IgG4-hinge format are shown in in FIG. 17.

TABLE 2
Exemplary Bispecific Antibodies

| | | VH | VL |
|---|---|---|---|
| | | SEQ | SEQ |
| Bispecific | | ID | ID |
| Ab No. | Description | NO. | NO. |
| 1 | BIIB-9-484 | 31 | 221 |
| | BIIB-12-1288 | 467 | 655 |
| 2 | BIIB-9-578 | 185 | 371 |
| | BIIB-12-1288 | 467 | 655 |
| 3 | BIIB-9-484 | 31 | 221 |
| | BIIB-12-1289 | 469 | 657 |
| 4 | BIIB-9-484 | 31 | 221 |
| | BIIB-12-1290 | 471 | 659 |
| 5 | BIIB-9-578 | 185 | 371 |
| | BIIB-12-1290 | 471 | 659 |
| 6 | BIIB-9-484 | 31 | 221 |
| | BIIB-12-1291 | 473 | 661 |
| 7 | BIIB-9-484 | 31 | 221 |
| | BIIB-12-1292 | 475 | 663 |
| 8 | BIIB-9-484 | 31 | 221 |
| | BIIB-12-1293 | 477 | 665 |
| 9 | BIIB-9-484 | 31 | 221 |
| | BIIB-12-1294 | 479 | 667 |
| 10 | BIIB-9-578 | 185 | 371 |
| | BIIB-12-1294 | 479 | 667 |
| 11 | BIIB-9-484 | 31 | 221 |
| | BIIB-12-1295 | 481 | 669 |
| 12 | BIIB-9-578 | 185 | 371 |
| | BIIB-12-1295 | 481 | 669 |
| 13 | BIIB-9-484 | 31 | 221 |
| | BIIB-12-1296 | 483 | 671 |
| 14 | BIIB-9-578 | 185 | 371 |
| | BIIB-12-1296 | 483 | 671 |
| 15 | BIIB-9-619 | 45 | 235 |
| | BIIB-12-1296 | 483 | 671 |
| 16 | BIIB-9-484 | 31 | 221 |
| | BIIB-12-1297 | 485 | 673 |
| 17 | BIIB-9-578 | 185 | 371 |
| | BIIB-12-1297 | 485 | 673 |
| 18 | BIIB-9-484 | 31 | 221 |
| | BIIB-12-1298 | 487 | 675 |
| 19 | BIIB-9-578 | 185 | 371 |
| | BIIB-12-1298 | 487 | 675 |
| 20 | BIIB-9-484 | 31 | 221 |
| | BIIB-12-1299 | 489 | 677 |
| 21 | BIIB-9-578 | 185 | 371 |
| | BIIB-12-1299 | 489 | 677 |
| 22 | BIIB-9-484 | 31 | 221 |
| | BIIB-12-1300 | 491 | 679 |
| 23 | BIIB-9-484 | 31 | 221 |
| | BIIB-12-1301 | 493 | 681 |
| 24 | BIIB-9-484 | 31 | 221 |
| | BIIB-12-1302 | 495 | 683 |
| 25 | BIIB-9-425 | 17 | 207 |
| | BIIB-12-1303 | 497 | 685 |
| 26 | BIIB-9-484 | 31 | 221 |
| | BIIB-12-1303 | 497 | 685 |
| 27 | BIIB-9-578 | 185 | 371 |
| | BIIB-12-1303 | 497 | 685 |
| 28 | BIIB-9-484 | 31 | 221 |
| | BIIB-12-1304 | 499 | 687 |
| 29 | BIIB-9-578 | 185 | 371 |
| | BIIB-12-1304 | 499 | 687 |
| 30 | BIIB-9-484 | 31 | 221 |
| | BIIB-12-1305 | 501 | 689 |
| 31 | BIIB-9-578 | 185 | 371 |
| | BIIB-12-1305 | 501 | 689 |
| 32 | BIIB-9-484 | 31 | 221 |
| | BIIB-12-1306 | 503 | 691 |
| 33 | BIIB-9-578 | 185 | 371 |
| | BIIB-12-1306 | 503 | 691 |
| 34 | BIIB-9-484 | 31 | 221 |
| | BIIB-12-1307 | 505 | 693 |
| 35 | BIIB-9-578 | 185 | 371 |
| | BIIB-12-1307 | 505 | 693 |
| 36 | BIIB-9-484 | 31 | 221 |
| | BIIB-12-1308 | 507 | 695 |
| 37 | BIIB-9-578 | 185 | 371 |
| | BIIB-12-1308 | 507 | 695 |
| 38 | BIIB-9-484 | 31 | 221 |
| | BIIB-12-1309 | 509 | 697 |
| 39 | BIIB-9-578 | 185 | 371 |
| | BIIB-12-1309 | 509 | 697 |
| 40 | BIIB-9-484 | 31 | 221 |
| | BIIB-12-1310 | 511 | 699 |
| 41 | BIIB-9-578 | 185 | 371 |
| | BIIB-12-1310 | 511 | 699 |
| 42 | BIIB-9-484 | 31 | 221 |
| | BIIB-12-1311 | 513 | 701 |
| 43 | BIIB-9-578 | 185 | 371 |
| | BIIB-12-1311 | 513 | 701 |
| 44 | BIIB-9-484 | 31 | 221 |
| | BIIB-12-1312 | 515 | 703 |
| 45 | BIIB-9-578 | 185 | 371 |
| | BIIB-12-1312 | 515 | 703 |
| 46 | BIIB-9-484 | 31 | 221 |
| | BIIB-12-1313 | 517 | 705 |
| 47 | BIIB-9-484 | 31 | 221 |
| | BIIB-12-1314 | 519 | 707 |
| 48 | BIIB-9-578 | 185 | 371 |
| | BIIB-12-1314 | 519 | 707 |
| 49 | BIIB-9-484 | 31 | 221 |
| | BIIB-12-1315 | 521 | 709 |
| 50 | BIIB-9-578 | 185 | 371 |
| | BIIB-12-1315 | 521 | 709 |
| 51 | BIIB-9-480 | 9 | 199 |
| | BIIB-12-1316 | 523 | 711 |
| 52 | BIIB-9-484 | 31 | 221 |
| | BIIB-12-1316 | 523 | 711 |
| 53 | BIIB-9-469 | 33 | 223 |
| | BIIB-12-1316 | 523 | 711 |
| 54 | BIIB-9-578 | 185 | 371 |
| | BIIB-12-1316 | 523 | 711 |
| 55 | BIIB-9-484 | 31 | 221 |
| | BIIB-12-1318 | 527 | 715 |
| 56 | BIIB-9-484 | 31 | 221 |
| | BIIB-12-1319 | 529 | 717 |
| 57 | BIIB-9-578 | 185 | 371 |
| | BIIB-12-1319 | 529 | 717 |
| 58 | BIIB-9-484 | 31 | 221 |
| | BIIB-12-1320 | 561 | 749 |
| 59 | BIIB-9-425 | 17 | 207 |
| | BIIB-12-1321 | 563 | 751 |
| 60 | BIIB-9-484 | 31 | 221 |
| | BIIB-12-1321 | 563 | 751 |
| 61 | BIIB-9-484 | 31 | 221 |
| | BIIB12-1322 | 531 | 719 |
| 62 | BIIB-9-484 | 31 | 221 |
| | BIIB-12-1323 | 533 | 721 |
| 63 | BIIB-9-484 | 31 | 221 |
| | BIIB-12-1325 | 537 | 725 |
| 64 | BIIB-9-578 | 185 | 371 |
| | BIIB-12-1325 | 537 | 725 |
| 65 | BIIB-9-484 | 31 | 221 |
| | BIIB-12-1326 | 539 | 727 |
| 66 | BIIB-9-578 | 185 | 371 |
| | BIIB-12-1326 | 539 | 727 |
| 67 | BIIB-9-484 | 31 | 221 |
| | BIIB-12-1327 | 541 | 729 |
| 68 | BIIB-9-578 | 185 | 371 |
| | BIIB-12-1327 | 543 | 731 |
| 69 | BIIB-9-484 | 31 | 221 |
| | BIIB-12-1329 | 545 | 733 |
| 70 | BIIB-9-484 | 31 | 221 |
| | BIIB-12-1330 | 547 | 735 |
| 71 | BIIB-9-631 | 187 | 373 |
| | BIIB-12-1330 | 547 | 735 |
| 72 | BIIB-9-484 | 31 | 221 |
| | BIIB-12-1331 | 549 | 737 |
| 73 | BIIB-9-484 | 31 | 221 |
| | BIIB-12-1332 | 551 | 739 |
| 74 | BIIB-9-484 | 31 | 221 |
| | BIIB-12-1333 | 553 | 741 |
| 75 | BIIB-9-416 | 93 | 279 |
| | BIIB-12-1334 | 555 | 743 |
| 76 | BIIB-9-484 | 31 | 221 |
| | BIIB-12-1334 | 555 | 743 |
| 77 | BIIB-9-578 | 185 | 371 |
| | BIIB-12-1334 | 555 | 743 |
| 78 | BIIB-9-484 | 31 | 221 |
| | BIIB-12-891 | 377 | 565 |
| 79 | BIIB-9-484 | 31 | 221 |
| | BIIB-12-892 | 379 | 567 |
| 80 | BIIB-9-484 | 31 | 221 |
| | BIIB-12-893 | 381 | 569 |
| 81 | BIIB-9-484 | 31 | 221 |
| | BIIB-12-894 | 557 | 745 |
| 82 | BIIB-9-578 | 185 | 371 |
| | BIIB-12-894 | 557 | 745 |
| 83 | BIIB-9-484 | 31 | 221 |
| | BIIB-12-895 | 383 | 571 |
| 84 | BIIB-9-484 | 31 | 221 |
| | BIIB-12-896 | 385 | 573 |
| 85 | BIIB-9-484 | 31 | 221 |
| | BIIB-12-897 | 387 | 575 |
| 86 | BIIB-9-578 | 185 | 371 |
| | BIIB-12-897 | 387 | 575 |
| 87 | BIIB-9-619 | 45 | 235 |
| | BIIB-12-897 | 387 | 575 |
| 88 | BIIB-9-628 | 113 | 299 |
| | BIIB-12-897 | 387 | 575 |
| 89 | BIIB-9-484 | 31 | 221 |
| | BIIB-12-898 | 389 | 577 |
| 90 | BIIB-9-484 | 31 | 221 |
| | BIIB-12-899 | 391 | 579 |
| 91 | BIIB-9-484 | 31 | 221 |
| | BIIB-12-900 | 393 | 581 |
| 92 | BIIB-9-484 | 31 | 221 |
| | BIIB-12-901 | 395 | 583 |
| 93 | BIIB-9-484 | 31 | 221 |
| | BIIB-12-902 | 397 | 585 |
| 94 | BIIB-9-484 | 31 | 221 |
| | BIIB-12-903 | 399 | 587 |
| 95 | BIIB-9-578 | 185 | 371 |
| | BIIB-12-903 | 399 | 587 |
| 96 | BIIB-9-619 | 45 | 235 |
| | BIIB-12-903 | 399 | 587 |
| 97 | BIIB-9-484 | 31 | 221 |
| | BIIB-12-904 | 401 | 589 |
| 98 | BIIB-9-484 | 31 | 221 |
| | BIIB-12-905 | 403 | 591 |
| 99 | BIIB-9-578 | 185 | 371 |
| | BIIB-12-905 | 403 | 591 |
| 100 | BIIB-9-484 | 31 | 221 |
| | BIIB-12-906 | 405 | 593 |
| 101 | BIIB-9-578 | 185 | 371 |
| | BIIB-12-906 | 405 | 593 |
| 102 | BIIB-9-619 | 45 | 235 |
| | BIIB-12-906 | 405 | 593 |
| 103 | BIIB-9-484 | 31 | 221 |
| | BIIB-12-907 | 407 | 595 |
| 104 | BIIB-9-484 | 31 | 221 |
| | BIIB-12-908 | 409 | 597 |
| 105 | BIIB-9-484 | 31 | 221 |
| | BIIB-12-909 | 411 | 599 |
| 106 | BIIB-9-484 | 31 | 221 |
| | BIIB-12-910 | 413 | 601 |
| 107 | BIIB-9-484 | 31 | 221 |
| | BIIB-12-911 | 415 | 603 |
| 108 | BIIB-9-484 | 31 | 221 |
| | BIIB-12-912 | 417 | 605 |
| 109 | BIIB-9-484 | 31 | 221 |
| | BIIB-12-913 | 419 | 607 |
| 110 | BIIB-9-484 | 31 | 221 |
| | BIIB-12-914 | 421 | 609 |
| 111 | BIIB-9-484 | 31 | 221 |
| | BIIB-12-915 | 423 | 611 |
| 112 | BIIB-9-578 | 185 | 371 |
| | BIIB-12-915 | 423 | 611 |
| 113 | BIIB-9-619 | 45 | 235 |
| | BIIB-12-915 | 423 | 611 |
| 114 | BIIB-9-484 | 31 | 221 |
| | BIIB-12-916 | 425 | 613 |
| 115 | BIIB-9-578 | 185 | 371 |
| | BIIB-12-916 | 425 | 613 |
| 116 | BIIB-9-607 | 99 | 285 |
| | BIIB-12-917 | 427 | 615 |
| 117 | BIIB-9-484 | 31 | 221 |
| | BIIB-12-917 | 427 | 615 |
| 118 | BIIB-9-578 | 185 | 371 |
| | BIIB-12-917 | 427 | 615 |
| 119 | BIIB-9-619 | 45 | 235 |
| | BIIB-12-917 | 427 | 615 |
| 120 | BIIB-9-484 | 31 | 221 |
| | BIIB-12-918 | 429 | 617 |
| 121 | BIIB-9-578 | 185 | 371 |
| | BIIB-12-918 | 429 | 617 |
| 122 | BIIB-9-484 | 31 | 221 |
| | BIIB-12-920 | 433 | 621 |
| 123 | BIIB-9-484 | 31 | 221 |
| | BIIB-12-921 | 435 | 623 |
| 124 | BIIB-9-484 | 31 | 221 |
| | BIIB-12-922 | 437 | 625 |
| 125 | BIIB-9-484 | 31 | 221 |
| | BIIB-12-923 | 439 | 627 |
| 126 | BIIB-9-578 | 185 | 371 |
| | BIIB-12-923 | 439 | 627 |
| 127 | BIIB-9-484 | 31 | 221 |
| | BIIB-12-924 | 441 | 629 |
| 128 | BIIB-9-578 | 185 | 371 |
| | BIIB-12-924 | 441 | 629 |
| 129 | BIIB-9-607 | 99 | 285 |
| | BIIB-12-925 | 559 | 747 |
| 130 | BIIB-9-439 | 105 | 291 |
| | BIIB-12-925 | 559 | 747 |
| 131 | BIIB-9-564 | 29 | 219 |
| | BIIB-12-925 | 559 | 747 |
| 132 | BIIB-9-484 | 31 | 221 |
| | BIIB-12-925 | 559 | 747 |
| 133 | BIIB-9-578 | 185 | 371 |
| | BIIB-12-925 | 559 | 747 |
| 134 | BIIB-9-615 | 111 | 297 |
| | BIIB-12-925 | 559 | 747 |
| 135 | BIIB-9-619 | 45 | 235 |
| | BIIB-12-925 | 559 | 747 |
| 136 | BIIB-9-628 | 113 | 299 |
| | BIIB-12-925 | 559 | 747 |
| 137 | BIIB-9-484 | 31 | 221 |
| | BIIB-12-926 | 443 | 631 |
| 138 | BIIB-9-484 | 31 | 221 |
| | BIIB-12-927 | 445 | 633 |
| 139 | BIIB-9-484 | 31 | 221 |
| | BIIB-12-927 | 445 | 633 |
| 140 | BIIB-9-484 | 31 | 221 |
| | BIIB-12-929 | 449 | 637 |
| 141 | BIIB-9-484 | 31 | 221 |
| | BIIB-12-930 | 451 | 639 |
| 142 | BIIB-9-484 | 31 | 221 |
| | BIIB-12-931 | 453 | 641 |
| 143 | BIIB-9-484 | 31 | 221 |
| | BIIB-12-933 | 457 | 645 |
| 144 | BIIB-9-484 | 31 | 221 |
| | BIIB-12-936 | 463 | 651 |
| 145 | BIIB-9-484 | 31 | 221 |
| | BIIB-12-937 | 465 | 653 |
| 146 | BIIB-9-1284 | 175 | 361 |
| | BIIB-12-892 | 379 | 567 |
| 147 | BIIB-9-451 | 53 | 243 |
| | BIIB-12-907 | 407 | 595 |
| 148 | BIIB-9-1265 | 137 | 323 |
| | BIIB-12-908 | 409 | 597 |
| 149 | BIIB-9-1275 | 157 | 343 |
| | BIIB-12-908 | 409 | 597 |
| 150 | BIIB-9-419 | 51 | 241 |
| | BIIB-12-910 | 413 | 601 |
| 151 | BIIB-9-473 | 55 | 245 |
| | BIIB-12-910 | 413 | 601 |
| 152 | BIIB-9-573 | 59 | 249 |
| | BIIB-12-910 | 413 | 601 |
| 153 | BIIB-9-581 | 63 | 253 |
| | BIIB-12-910 | 413 | 601 |
| 154 | BIIB-9-582 | 65 | 255 |
| | BIIB-12-910 | 413 | 601 |
| 155 | BIIB-9-592 | 73 | 263 |
| | BIIB-12-910 | 413 | 601 |
| 156 | BIIB-9-608 | 77 | 267 |
| | BIIB-12-910 | 413 | 601 |
| 157 | BIIB-9-612 | 189 | 375 |
| | BIIB-12-910 | 413 | 601 |
| 158 | BIIB-9-616 | 79 | 269 |
| | BIIB-12-910 | 413 | 601 |
| 159 | BIIB-9-1265 | 137 | 323 |
| | BIIB-12-910 | 413 | 601 |
| 160 | BIIB-9-1268 | 143 | 329 |
| | BIIB-12-910 | 413 | 601 |
| 161 | BIIB-9-1269 | 145 | 331 |
| | BIIB-12-910 | 413 | 601 |
| 162 | BIIB-9-1272 | 151 | 337 |
| | BIIB-12-910 | 413 | 601 |
| 163 | BIIB-9-1273 | 153 | 339 |
| | BIIB-12-910 | 413 | 601 |
| 164 | BIIB-9-1274 | 155 | 341 |
| | BIIB-12-910 | 413 | 601 |
| 165 | BIIB-9-1275 | 157 | 343 |
| | BIIB-12-910 | 413 | 601 |
| 166 | BIIB-9-1276 | 159 | 345 |
| | BIIB-12-910 | 413 | 601 |
| 167 | BIIB-9-1279 | 165 | 351 |
| | BIIB-12-910 | 413 | 601 |
| 168 | BIIB-9-1282 | 171 | 357 |
| | BIIB-12-910 | 413 | 601 |
| 169 | BIIB-9-1284 | 175 | 361 |
| | BIIB-12-910 | 413 | 601 |
| 170 | BIIB-9-1285 | 177 | 363 |
| | BIIB-12-910 | 413 | 601 |
| 171 | BIIB-9-1278 | 163 | 349 |
| | BIIB-12-913 | 419 | 607 |
| 172 | BIIB-9-1285 | 177 | 363 |
| | BIIB-12-913 | 419 | 607 |
| 173 | BIIB-9-590 | 71 | 261 |
| | BIIB-12-914 | 421 | 609 |
| 174 | BIIB-9-612 | 189 | 375 |
| | BIIB-12-921 | 435 | 623 |
| 175 | BIIB-9-621 | 81 | 271 |
| | BIIB-12-925 | 559 | 747 |
| 176 | BIIB-9-1265 | 137 | 323 |
| | BIIB-12-925 | 559 | 747 |
| 177 | BIIB-9-1275 | 157 | 343 |
| | BIIB-12-925 | 559 | 747 |
| 178 | BIIB-9-1279 | 165 | 351 |
| | BIIB-12-925 | 559 | 747 |
| 179 | BIIB-9-1265 | 137 | 323 |
| | BIIB-12-1288 | 467 | 655 |
| 180 | BIIB-9-1275 | 157 | 343 |
| | BIIB-12-1288 | 467 | 655 |
| 181 | BIIB-9-451 | 53 | 243 |
| | BIIB-12-1292 | 475 | 663 |
| 182 | BIIB-9-573 | 59 | 249 |
| | BIIB-12-1292 | 475 | 663 |
| 183 | BIIB-9-581 | 63 | 253 |
| | BIIB-12-1292 | 475 | 663 |
| 184 | BIIB-9-582 | 65 | 255 |
| | BIIB-12-1292 | 475 | 663 |
| 185 | BIIB-9-585 | 67 | 257 |
| | BIIB-12-1292 | 475 | 663 |
| 186 | BIIB-9-608 | 77 | 267 |
| | BIIB-12-1294 | 479 | 667 |
| 187 | BIIB-9-1265 | 137 | 323 |
| | BIIB-12-1294 | 479 | 667 |
| 188 | BIIB-9-1265 | 137 | 323 |
| | BIIB-12-1297 | 485 | 673 |
| 189 | BIIB-9-608 | 77 | 267 |
| | BIIB-12-1300 | 491 | 679 |
| 190 | BIIB-9-1265 | 137 | 323 |
| | BIIB-12-1300 | 491 | 679 |
| 191 | BIIB-9-608 | 77 | 267 |
| | BIIB-12-1301 | 493 | 681 |
| 192 | BIIB-9-1265 | 137 | 323 |
| | BIIB-12-1301 | 493 | 681 |
| 193 | BIIB-9-1265 | 137 | 323 |
| | BIIB-12-1312 | 515 | 703 |
| 194 | BIIB-9-433 | 127 | 313 |
| | BIIB-12-1314 | 519 | 707 |
| 195 | BIIB-9-1265 | 137 | 323 |
| | BIIB-12-1314 | 519 | 707 |
| 196 | BIIB-9-1275 | 157 | 343 |
| | BIIB-12-1314 | 519 | 707 |
| 197 | BIIB-9-1276 | 159 | 345 |
| | BIIB-12-1314 | 519 | 707 |
| 198 | BIIB-9-1276 | 159 | 345 |
| | BIIB-12-1315 | 521 | 709 |
| 199 | BIIB-9-1268 | 143 | 329 |
| | BIIB-12-1318 | 527 | 715 |
| 200 | BIIB-9-1265 | 137 | 323 |
| | BIIB-12-1319 | 529 | 717 |
| 201 | BIIB-9-1284 | 175 | 361 |
| | BIIB-12-1327 | 541 | 729 |
| 202 | BIIB-9-1273 | 153 | 339 |
| | BIIB-12-1334 | 555 | 743 |

### Example 4

### Assessment of Bispecific Antibodies in Plasma-Based Coagulation Assays

Bispecific antibodies that were able to replace and/or mimic FVIIIa activity in a chromogenic assay were further tested for their ability to replace FVIIIa-like activity in a one stage clotting assay in plasma. Only those bispecific antibodies displaying the greatest activity in the chromogenic assay described above were selected for this study.

Bispecific antibodies (5 μL) at varying concentrations were mixed with 50 μL of FVIII-deficient plasma (Siemens) for 60 seconds. To activate the reaction, 50 μL of Actin FSL Ellagic Acid was added to the reaction mixture for 240 seconds, followed by 50 μL of CaCl₂. Time to clot was measured for 300 seconds by optical detection using a Sysmex CA-1500 system (Siemens). A decrease in clotting time in the presence of a bispecific antibody, specifically a decrease in clotting time beyond baseline to approximately 126 seconds and a dose response indicating a decrease in clotting time, is considered indicative of the ability of the antibody to replace FVIIIa function and to facilitate clot formation.

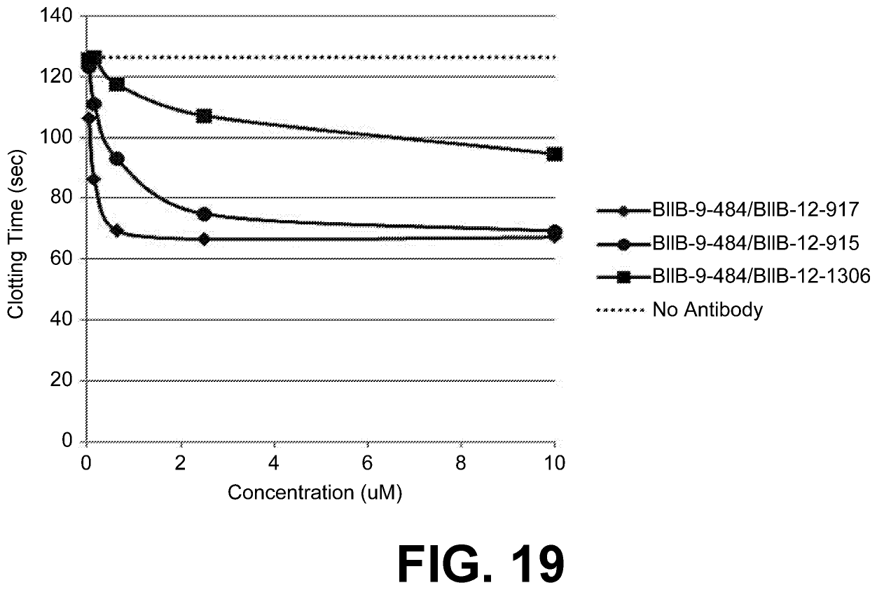

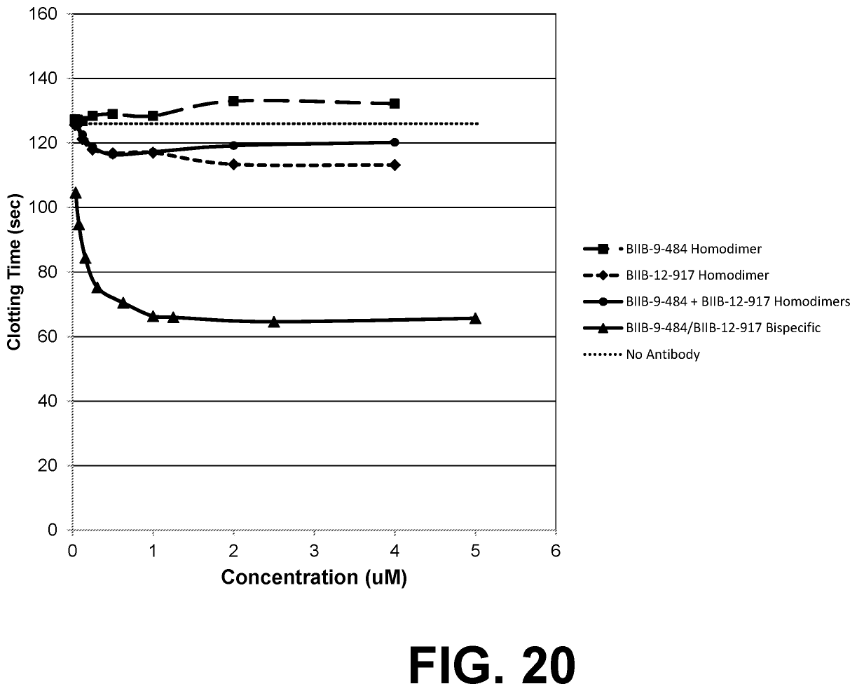

FIG. 19 shows an example of three such bispecific antibodies: BIIB-9-484/BIIB-12-917, BIIB-9-484/BIIB-12-915, and BIIB-9-484/BIIB-12-1306. To show that the FVIIIa-like activity results from the bispecific format of the antibody, the same experiment was performed with either an anti-FIXa and anti-FX homodimer alone, or as a mixed population of homodimers compared to the bispecific antibody. Results for the BIIB-9-484 and BIIB-12-917 pair are shown in FIG. 20.

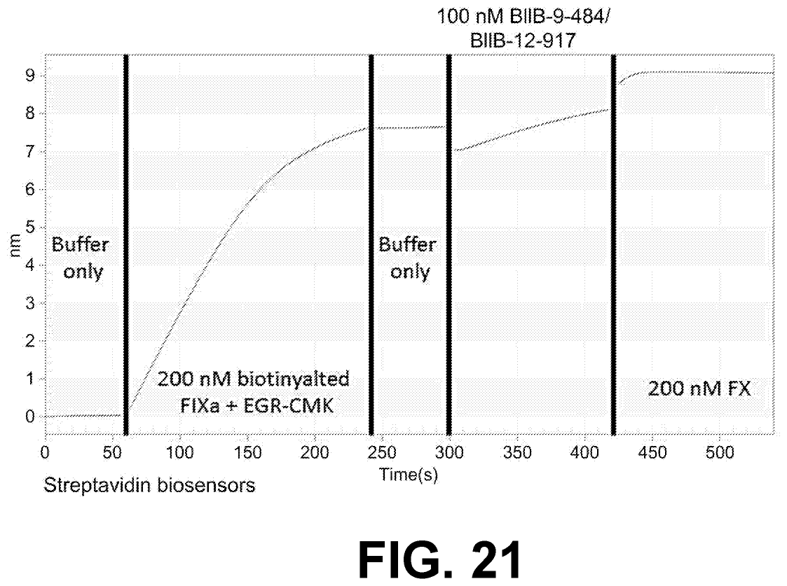

To ensure that the bispecific activity observed in FXa generation and plasma based coagulation assays involved simultaneous association of each of the target antigens, a BLI-based experiment was performed on the ForteBio HTX system using streptavidin dip and read biosensors (Pall ForteBio; Catalog No. 18-5021). Following incubation in Tris-buffered saline supplemented with 0.1% bovine serum albumin and 5 mM CaCl₂ (referred to as buffer in FIG. 21), the biosensors were then incubated with biotinylated FIXa-SM produced in-house. Subsequently, the biosensors were transferred to wells containing 100 nM of BIIB-9-484/BIIB-12-917. Finally, the same biosensors were incubated with 200 nM of non-biotinylated FX (non-activated Factor X). As demonstrated in the disclosure herein, the bispecific BIIB-9-484/BIIB-12-917 was capable of associating with each of the target antigens, presented as an example in FIG. 21. This is supportive of, and in agreement with, its observed functional activity.

### Example 5

### Engineering and Optimization of Bispecific Activity

To determine whether bispecific activity could be improved through engineering of an antibody:antigen interaction, the CDR1 and CDR2 regions of the BIIB-9-484 VH were modified to introduce amino acid diversity into the germline sequence. A library of 10⁶ sequence derivatives of BIIB-9-484 was then introduced into the Adimab platform.

To identify derivatives with improved affinity to FIXa (i.e., free FXa) and FIXa-SM relative to non-activatable FIX, expression libraries were subjected to several iterative rounds of selection with selective pressure applied towards higher affinity clones in accordance with methods disclosed in U.S. Publication Nos. 20100056386 and 20090181855, which are herein incorporated by reference in their entireties. See, also, Van Deventer and Wittrup (2014) Methods Mol. Biol. 1319:3-36; Chao et al. (2006), Nature Protocols 1(2):755-768; Feldhaus et al. (2003) Nature Biotechnology 21(2):163-170; Boder and Wittrup (1997) Nature Biotechnology 15(6):553-557, all of which are herein incorporated by reference in their entireties.

Subsequently, colonies were sequenced to identify unique derivatives, according to methods known in the art. This procedure identified at least 76 unique VH sequences. 76 antibodies were expressed and purified from yeast by protein A purification, according to standard procedures in the art.

76 unique derivative antibodies from the parental BIIB-9-484 were then re-screened for improved binding to target antigen, free FIXa (HTI) or FIXa-SM (HTI), this time using BLI in a monovalent assay format with 200 nM antibody. BLI was performed on the Octet HTX system according to manufacturer's procedures. Briefly, anti-Human IgG Quantitation (AHQ) dip and read biosensors (Pall Fortebio: Cat. No. 18-5005) were equilibrated in TBSFCa. Plates were then transferred to the instrument. A soak step was performed on the instrument, with incubation of the biosensors for 60 seconds in TBSFCa. Then, the biosensors were transferred to wells containing the representative IgG (100 nM in TBSFCa) for 180 seconds. Following this, the biosensors were incubated in TBSFCa for 60 seconds to establish baseline. Then, the biosensors were transferred to wells containing the desired antigen (10 nM in TBSFCa) and association occurred for 90-180 seconds. Finally, the sensors were transferred to TBSFCa alone for 180 second incubation.

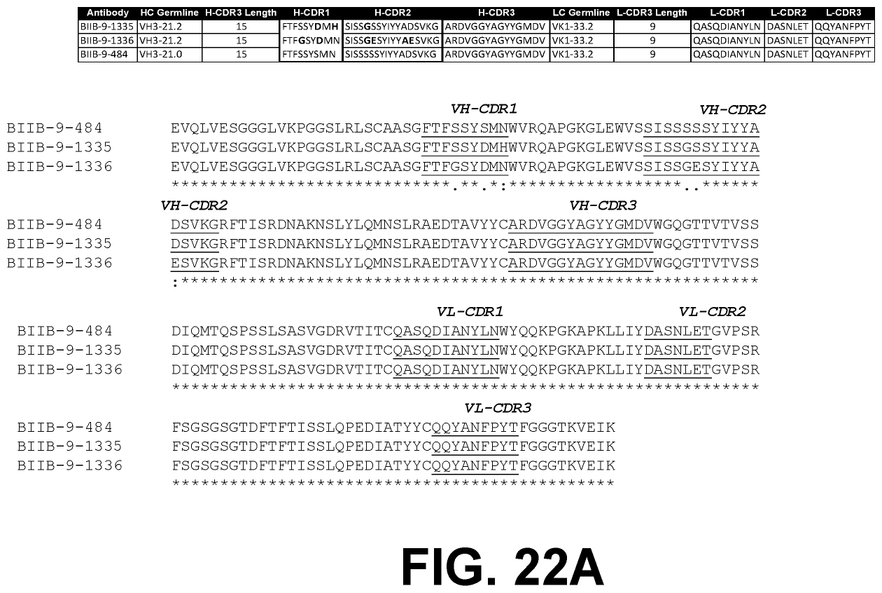

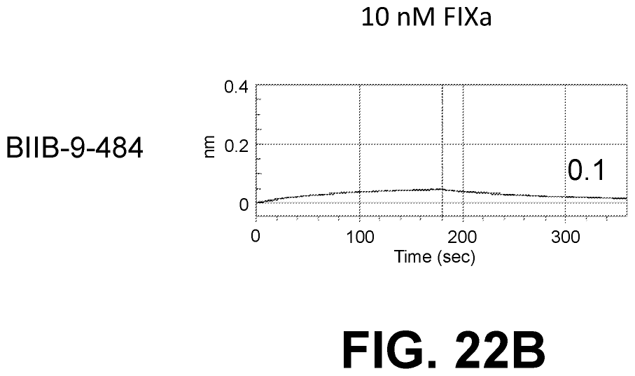

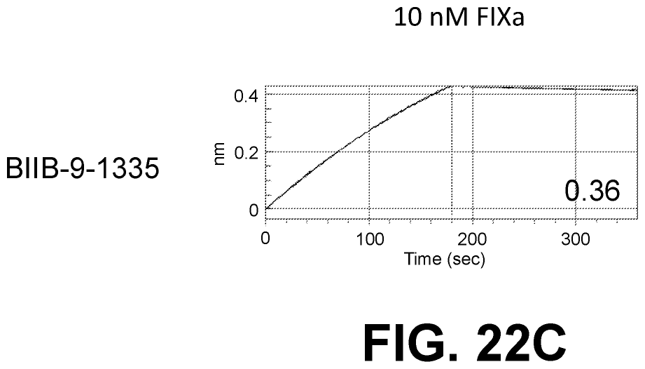

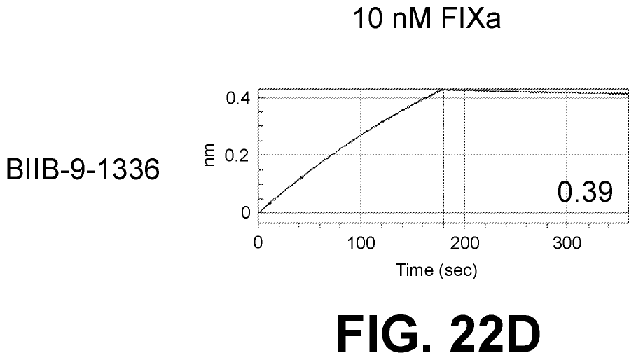

At least two derivatives of BIIB-9-484, named BIIB-9-1335 and BIIB-9-1336, respectively, demonstrated significantly improved binding to target antigen relative to the parent at a concentration of 10 nM antibody. Deviations from the BIIB-9-484 CDR1 and CDR2 regions of the heavy chain are noted in FIG. 22A. BLI binding profiles of the two derivatives are disclosed, exhibiting the stronger binding to target antigen (i.e., free FIXa) observed (FIG. 22B-22D). The amino acid and nucleic acid sequences of the modified VH regions of BIIB-9-484, BIIB-9-1335, and BIIB-9-1336 are provided below.

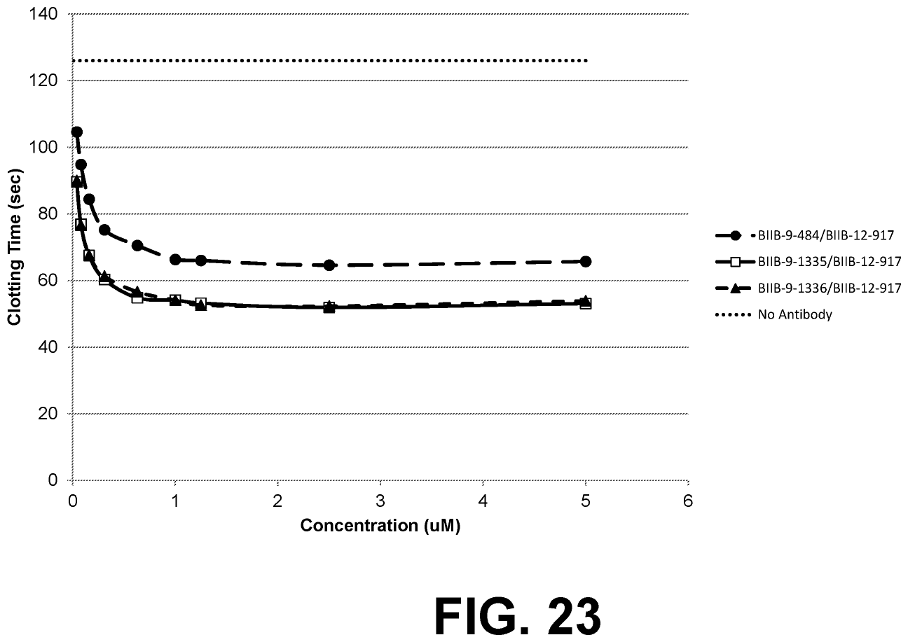

The effect of increasing the affinity of the anti-FIXa arm of the bispecific was tested by comparing the ability of each bispecific (containing either the parent anti-FIXa arm or the affinity matured anti-FIXa arm with a constant anti-FX arm) to replace FVIIIa activity in a one stage clotting assay. Experiments were performed as explained in Example 4. One such example is shown in FIG. 23, in which the anti-FX arm of the bispecific (BIIB-12-917) was paired with either the BIIB-9-484 anti-FIXa arm, or with the affinity matured daughters of the anti-FIXa arm (BIIB-9-1335 and BIIB-9-1336) (FIG. 23). Further decreases in the clotting time of the bispecific antibodies containing the affinity matured anti-FIXa arms versus the parent anti-FIXa arm indicate that increasing the affinity of the anti-FIXa arm results in an increase in activity of the resulting bispecific antibody.

### Example 6

### Benchmarking FVIIIa-Mimetic Bispecific Antibodies Against FVIII for Hemophilia A Treatment

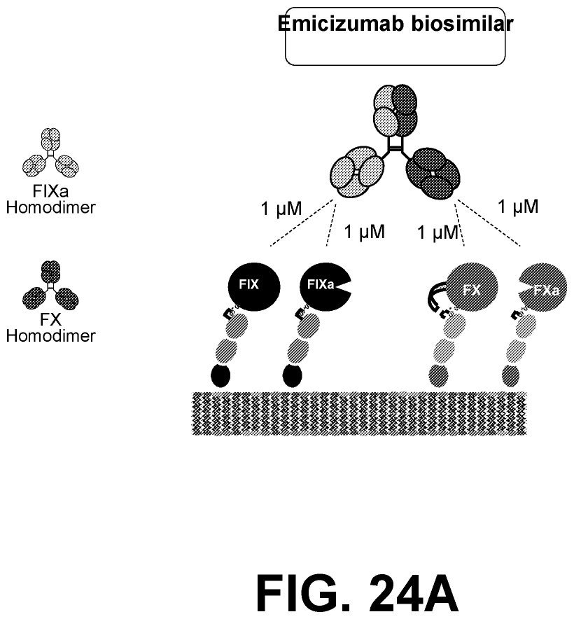

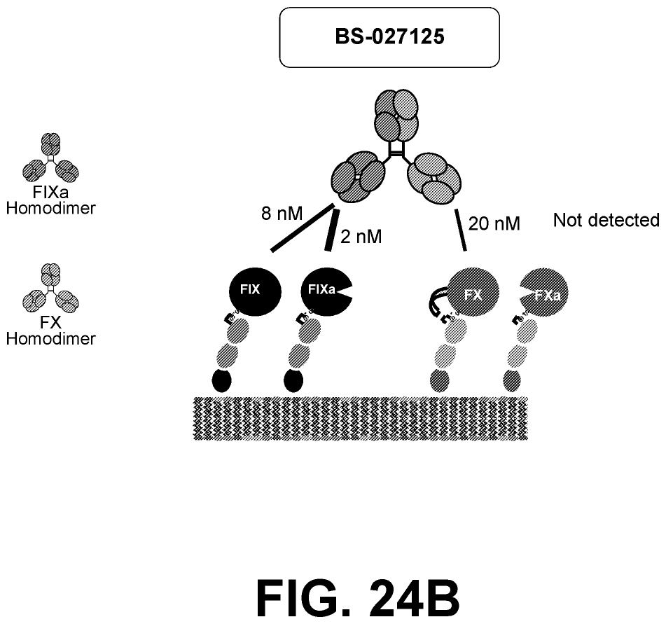

Using a number of activity assays, a reference bispecific antibody (bsAb) and a bispecific antibody of the present disclosure, BS-027125, were compared to recombinant FVIII (rFVIII). The bsAb BS-027125 comprises BIIB-9-1336 and BIIB-12-917. The reference bispecific antibody has a sequence identical to ACE910/emicizumab (“emicizumab biosimilar”); ACE910 is a recombinant humanized bispecific antibody that binds to activated factor IX and factor X and mimics the cofactor function of factor VIII (FVIII). ACE910 is disclosed in U.S. Pat. No. 8,062,635, which is incorporated herein by reference. Emicizumab biosimilar and BS-027125 are depicted in FIG. 24A and FIG. 24B, respectively. Emicizumab biosimilar binds to each of factor IX zymogen, factor IXa, factor X zymogen, and factor Xa with a K_{d} of approximately 1 μM (FIG. 24A). BS-027125 also binds to factor IX zymogen (K_{D}=8 nM), factor IXa (K_{D}=2 nM), and factor X zymogen (K_{D}=20 nM), but does not bind to factor Xa (FIG. 24B). Accordingly, BS-027125 has a higher affinity and greater specificity for activated factor IX and/or factor X zymogen than emicizumab biosimilar.

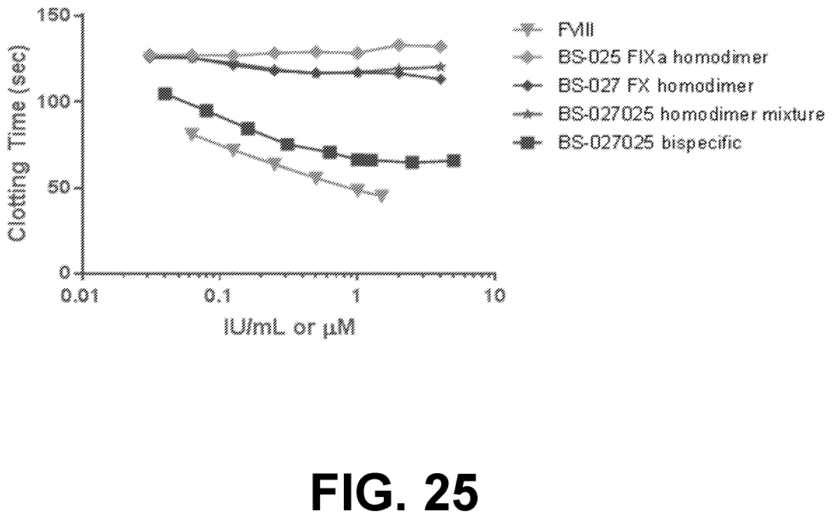

BS-027125 was designed as disclosed above using the Adimab in vitro yeast presentation platform with FACS-based selection followed by affinity maturation. The initial pool of bsAbs was generated by formatting the >200 unique antibodies specific for FIXa and the >250 unique antibodies specific for FX that were identified using Adimab into bsAbs. BS-027125's parent antibody, BS-027025, had the highest FVIIIa-like activity among this initial pool of bsAbs maintaining significant activity in a one-stage clotting assay. As depicted in FIG. 25, BS-027025 significantly shortened the clotting time, coming close to the activity of recombinant Factor VIII in the same assay. BS-027025 bound to factor XI zymogen (K_{D}=370 nM), factor IXa (K_{D}=67 nM), factor IXa-LTR (K_{D}=10.5 nM), and factor X zynmogen (K_{D}=20 nM), but not factor IXa as shown in Table 3.

TABLE 3

| Factor | BS-025 |
|---|---|
| Factor IX zymogen | 370 nM |
| Factor IXa | 67 nM |
| Factor IXa-LTR | 10.5 nM |
| | BS-027 |
| Factor X zymogen | 20 nM |
| Factor Xa | Not detected |

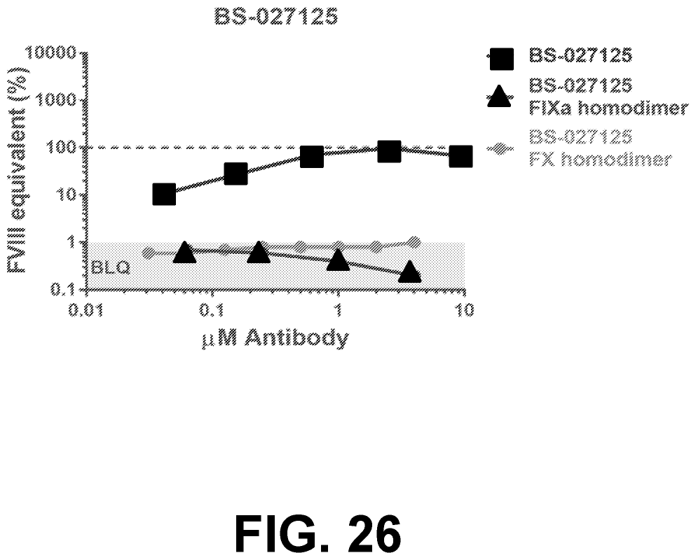

Affinity maturation of the anti-FIXa arm of BS-027025 led to the bsAb BS-027125. Increasing the affinity of the anti-FIXa arm (BS-025 to BS-125) increased the FXa generation rate of the resulting bispecific antibody, particularly when paired with BS-027 or BS-007 components in the bispecific antibody. (data not shown) Further testing indicated that BS-027125 achieved approximately 90% FVIIIa-like activity as determined by one-stage clotting assay (FIG. 26).

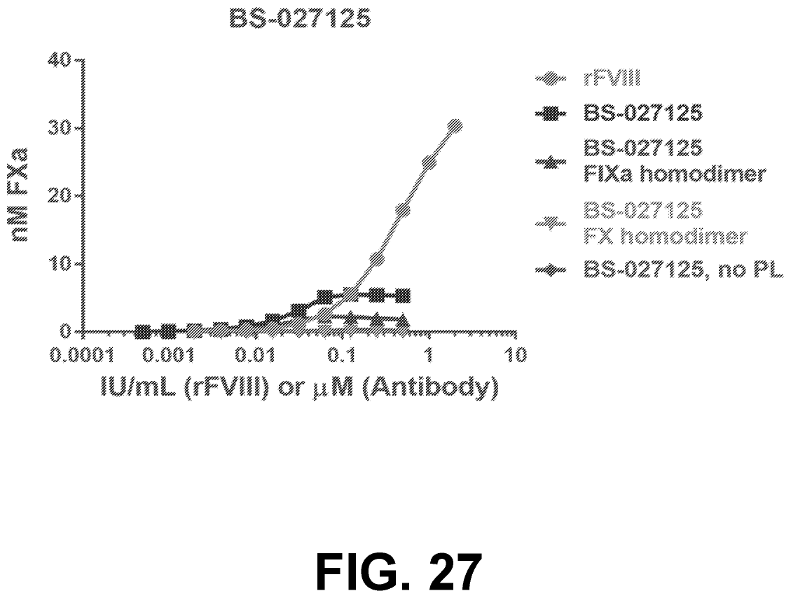

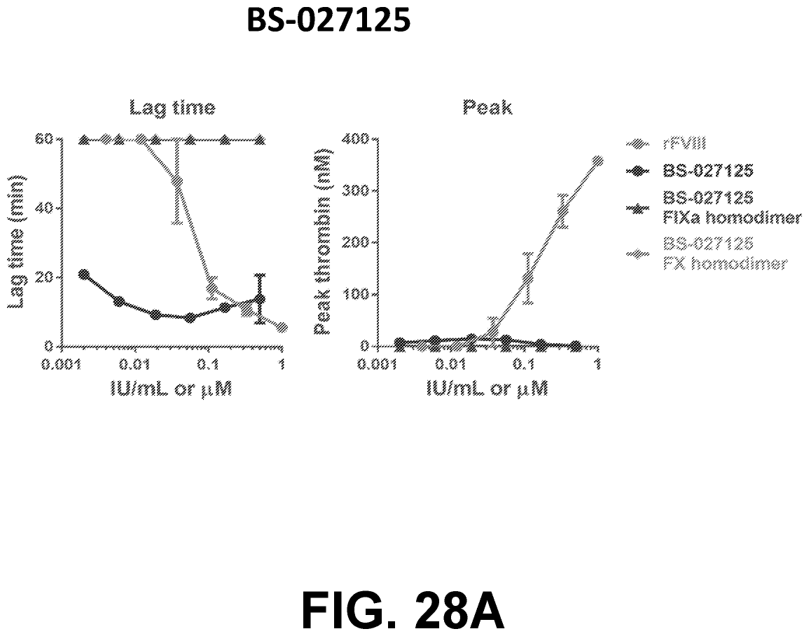

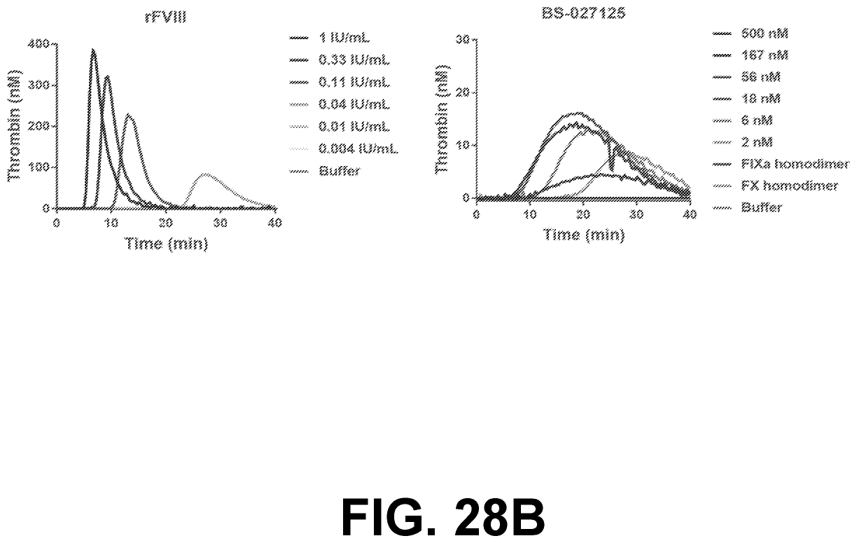

Activity of BS-027125, its respective bivalent homodimers, and rFVIII was measured by chromogenic factor Xa (FXa) generation assay (FIG. 27), thrombin generation assay triggered with factor XIa (FIGS. 28A and 28B), and activated partial thromboplastin time (aPTT) triggered with Actin FSL (FIG. 26).

Whereas the reference bsAb was highly active across all assays, the concentration at which it achieved peak activity was disparate between the assays. BS-027125 also displayed activity in all assays, and was highly active in aPTT. For both bsAbs, lag time and peak height in thrombin generation assay correlated with different levels of rFVIII activity. Both the reference bsAb bivalent homodimers exhibited significant activity in several assays, while only the BS-027125 FIXa bivalent homodimer retained moderate activity in the FXa generation assay. As expected, rFVIII lost all activity in the absence of phospholipids in FXa generation assay. BS-027125 also showed minimal phospholipid-independent activity. In contrast, the reference bsAb had very significant activity in the absence of phospholipids.

### Example 7

### Effect of Phospholipid Composition on Activity of Bispecific FVIIIa Mimetic Antibodies

As part of the tenase complex, activated factor VIII (FVIIIa) binds to exposed phosphatidylserine (PS) on cell membranes and assembles with activated factor IXa and factor X. It has been shown that FVIIIa binds preferentially to phospholipids containing both PS (phosphatidylserines) and phosphatidylethanolamine (PE). The presence of phosphatidylethanolamine (PE) in phosphatidylcholine (PC) phospholipid vesicles has been shown to reduce the amount of PS needed for optimal clotting factor activity whereas vesicles composed of only PS and PC require higher levels of PS for optimal activity. Recently, a FVIIIa mimetic bispecific antibody (emicizumab) has been developed as a potential treatment for hemophilia A patients with and without inhibitors. The FVIII-mimetic bispecific antibody BS-027125 has improved target specificity over emicizumab. Given that phospholipid composition influences tenase activity and that antibodies do not directly bind phospholipids, it is unclear whether FVIIIa mimetic antibodies will show similar phospholipid preferences.

The effect of varying phospholipid compositions and concentrations on the activity of the reference bsAb shown in Example 6, a bispecific antibody of the present disclosure, BS-027125, and recombinant FVIII (rFVIII) was compared.

The procoagulant activity of the reference bsAb, BS-027125 and rFVIII were assessed by a thrombin generation assay triggered with factor XIa. Unilamellar phospholipid vesicles were prepared by extrusion, as described in Mui B, et al. Methods Enzymol. 2003. Synthetic phospholipid vesicles tested were composed of either PS(phosphatidylserine)/PE(phosphatidylethanolamine)/PC(phosphatidylcholine) (20%/40%/40%) or PS/PC (20%/80%).

As expected, rFVIII activity was ˜2.5-fold higher on PE-containing phospholipids and activity was lost on both phospholipids when limiting or in extreme excess. Notably, whereas the reference bsAb activity was similar on both phospholipids, BS-027125 was ˜3 fold more active on PE-containing phospholipids. The phospholipid concentration that supported peak activity was higher for the reference bsAb and BS-027125 than for rFVIII. These results suggest the reference bsAb and BS-027125 function via different mechanisms.

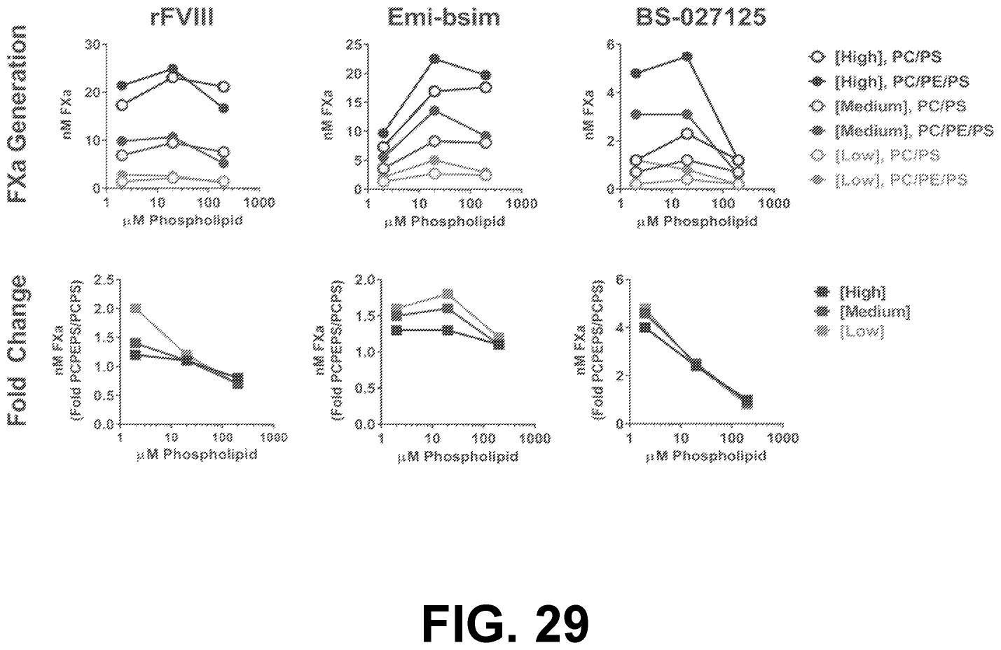

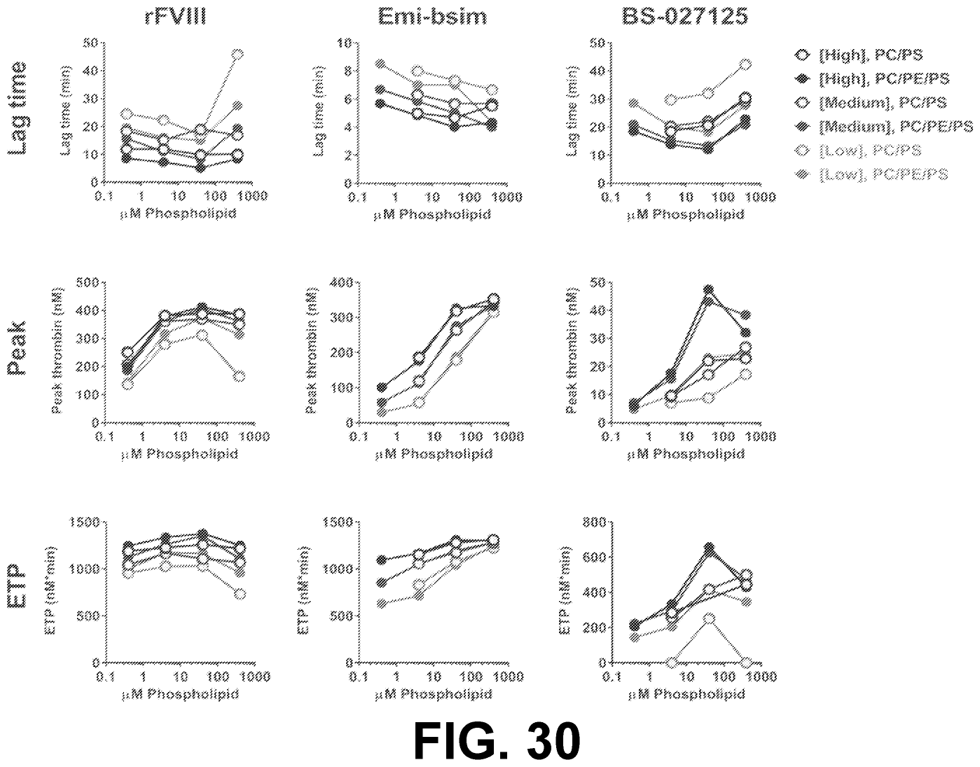

For rFVIII and BS-027125, the trend of increased activity on PE-containing phospholipids is maintained between FXa generation assay and thrombin generation assay. See FIG. 29. Emi-bsim, however, had increased activity in FXa generation, but not in thrombin generation. See FIG. 30. The different relative activities of rFVIII and FVIIIa mimetic bispecific antibodies on different phospholipid surfaces complicate direct comparisons between these molecules and highlight the influence of assay design when benchmarking the activity of FVIIIa mimetic bispecific antibodies to rFVIII. These data further suggest differences in mechanism of action between Emi-bsim and BS-027125.

### Example 8

### Epitope Binning of Anti-FIXa Antibodies

To determine whether the various pairs of anti-FIXa antibodies bind to unique sites, we performed a binning experiment using biolayer interferometry on the Octet HTX system following manufacturer's instruction. Briefly, anti-Human IgG Quantitation (AHQ) dip and read biosensors (Pall Fortebio: Catalog #18-5005) were equilibrated in TBSFCa for 60 seconds. Subsequently, the first antibody (at 200 nM in TBSFCa) was loaded onto the biosensor tip over 180 seconds, followed by a baseline step in buffer alone for 60 seconds.

Any free binding sites remaining on the probe were blocked with non-specific IgG's for 180 seconds, followed by another baseline step in buffer alone for 60 seconds. Next, FIXa+SM (100 nM in TBSFCa) was allowed to bind to the anti-FIXa antibody-loaded probes for 90 seconds. Finally, complex between the first antibody and FIXa+SM was exposed to the second anti-FIXa antibody (at 200 nM in TBSFCa).

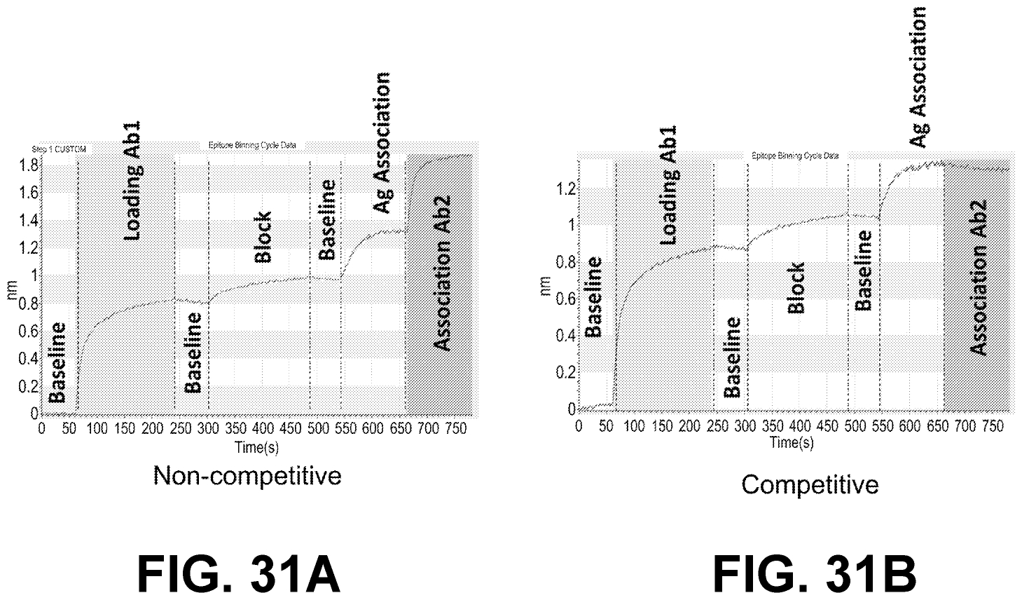

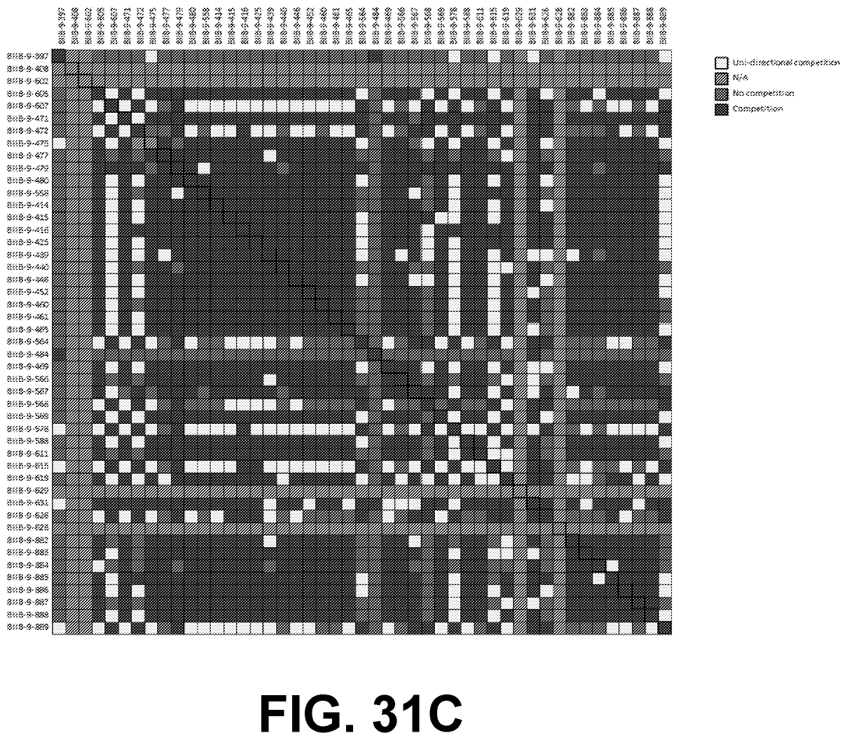

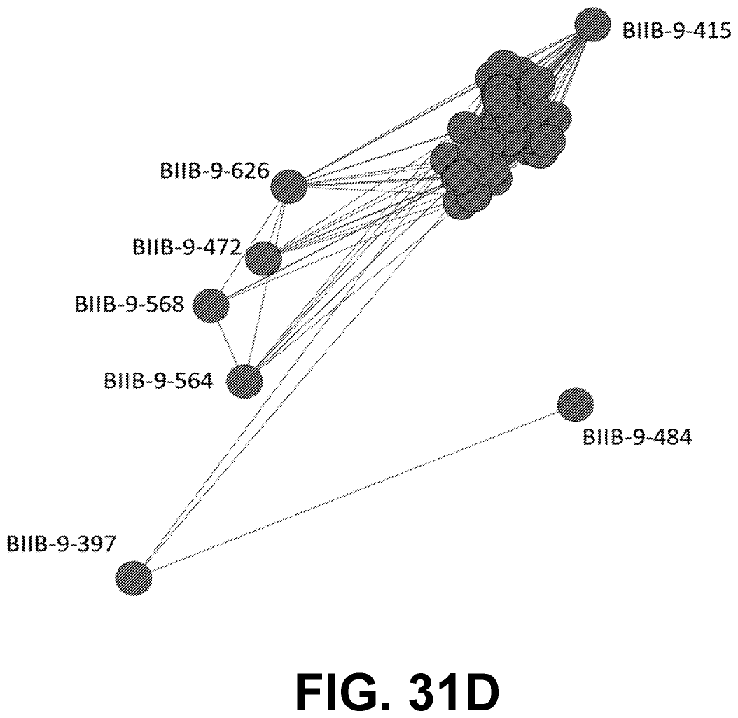

A further increase in signal indicated that antibody 1 and antibody 2 could bind to the antigen simultaneously and that they were non-competitive, meaning that they did not fall into the same bin (FIG. 31A). However, if no further increase in signal was observed, this indicated that antibody 1 and antibody 2 could not bind to the antigen simultaneously and therefore they were said to compete, meaning that they fell into the same in (FIG. 31B).

If simultaneous binding of antibody 1 and 2 was only observed in one direction (i.e. antibody1-antigen-antibody2 versus antibody 2-antigen-antibody 1), this was deemed a uni-directional conflict.

A subset of 48 antibodies were chosen for this analysis and the results are summarized in FIG. 31C. A few antibodies were dropped from the analysis owing to errors in data collection or because the given antibody did not bind to FIXa+SM (e.g, because it was specific for FIXa).

A binning network was subjected to node analysis to provide a visual representation of how closely related each of the FIXa antibodies are with regard to their binning profiles (FIG. 31D). The majority of the antibodies cluster very closely to one another, while there are a few distinct groups. BIIB-9-484 appeared to fall into a unique bin.

### Example 9

### Calcium Dependent Binding of BIIB-9-484 and BIIB-9-1336

Owing to the unique binning profile of BIIB-9-484, we investigated additional properties of this antibody as well as an affinity matured daughter, BIIB-9-1336. Since the activity and binding properties of many coagulation factors, including for FIXa, are calcium dependent, we first tested whether the binding of these two antibodies was affected by the presence or absence of calcium using biolayer interferometry (BLI).

Using the Octet QK384 system anti-Human IgG Quantitation (AHQ) dip and read biosensors (Pall Fortebio: Catalog #18-5005) were equilibrated in HBS for 60 seconds, followed by a 180 second loading step of the antibodies at 10ug/mL onto the probes. After a 60 second baseline step in HBS, the antibody-loaded probes were exposed to 200 nM FIXa in HBS or HBS with 5 mM CaCl₂) for 180 seconds, followed by a dissociation step in HBS or HBS with 5 mM CaCl₂) alone for 180 seconds.

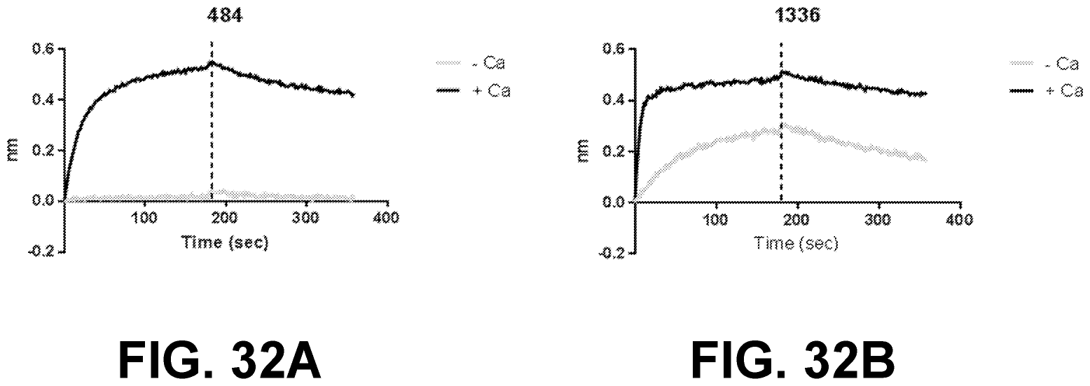

The binding data shown in FIGS. 32A and 32B indicates that the binding of both BIIB-9-484 and BIIB-9-1336 to FIXa is calcium-dependent. BIIB-9-484 is completely dependent on the presence of calcium, while the binding of BIIB-9-1336 to FIXa is significantly reduced, but still measurable, in the absence of calcium.

### Example 10

### Effect of BIIB-9-1336 on Proteolytic Activity of FIXa

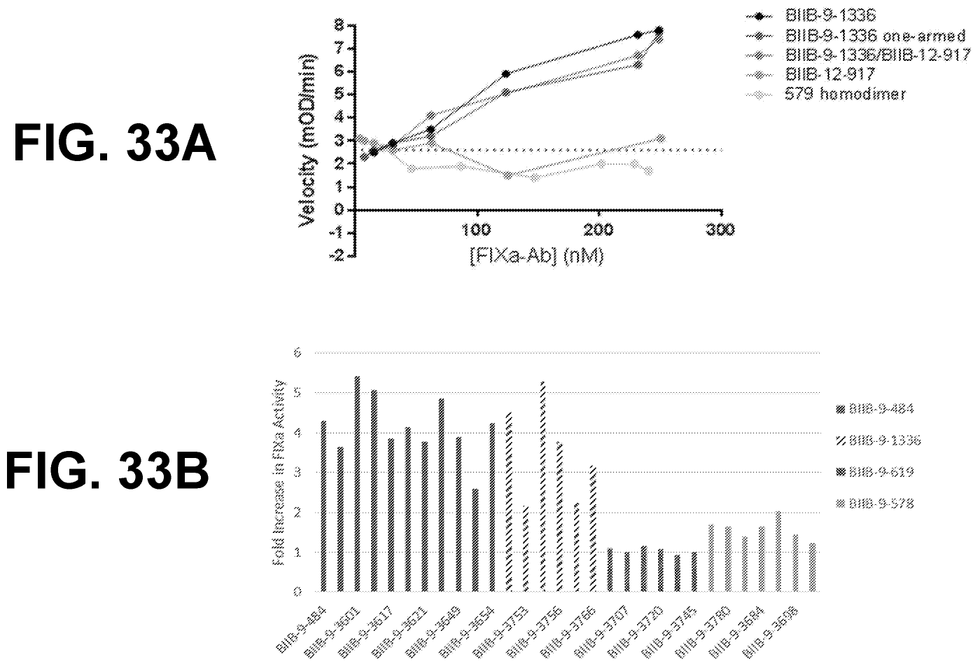

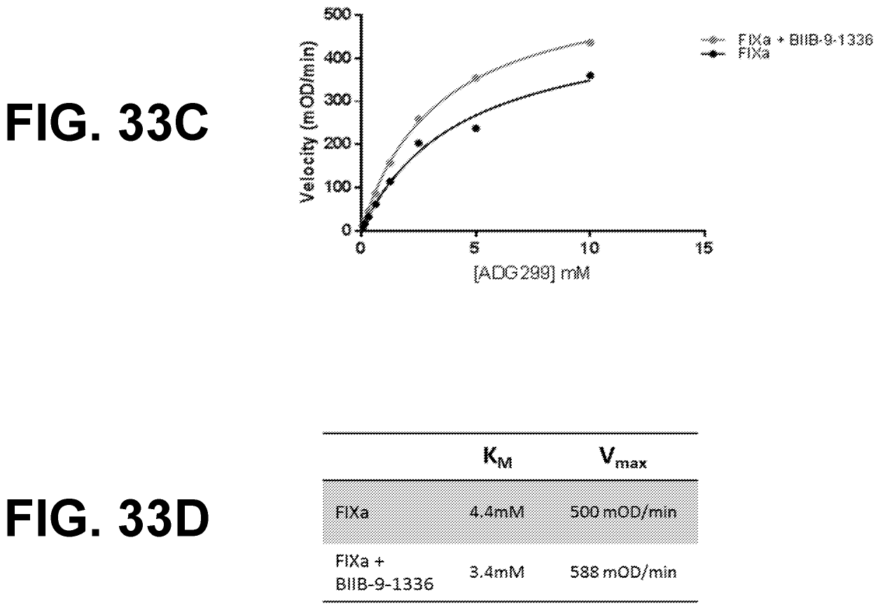

To determine whether BIIB-9-1336 has an effect on the enzymatic function of FIXa, we incubated varying amounts of antibody with 250 nM of FIXa in TBSCa for 5 minutes. Subsequently a peptide substrate, ADG299, was added to a final concentration of 0.8 mM and the rate of substrate cleavage by FIXa was measured by the change in OD over time (FIG. 33A).

For comparison, another anti-FIXa antibody, BIIB-9-579, was also tested for its ability to affect the amidolytic activity of FIXa, as well as an anti-FX antibody, BIIB-12-917, as a negative control. BIIB-9-1336 was able to increase the rate of substrate cleavage by FIXa 3-fold, while BIIB-9-579 and BIIB-12-917 had no effect.

This activity is not dependent on the homodimeric nature of BIIB-9-1336 as the one-armed and bispecific antibodies, which contain only one BIIB-9-1336 arm, showed the same 3-fold increase in rate. Since BIIB-9-1336 is a daughter of BIIB-9-484 and falls into the same unique bin, we continued to test other antibodies in this bin for the ability to increase the amidolytic activity of FIXa. Using the same assay set-up as described above, though using 500 nM FIXa, we tested 15 antibodies from the BIIB-9-484/1336 bin and an additional 11 daughter antibodies from the BIIB-9-619 and BIIB-9-578, which fall into different bins.

The fold-increase in amidolytic activity of FIXa in the presence of these antibodies is shown in FIG. 33B, and indicates that the ability to increase the amidolytic activity of FIXa is unique to antibodies form the BIIB-9-484/BIIB-9-1336 bin, reaching up to a 5-fold increase under the conditions tested. We next determined the kinetic parameters, KM and Vmax, of FIXa towards ADG299 in the presence an absence of BIIB-9-1336. Here, 500 nM of FIXa was incubated with 1000 nM of BIIB-9-1336 in TBSCa plus 33% ethylene glycol. The concentration of the substrate ADG299 was varied from 10 mM to 0.078 mM. Addition of BIIB-9-1336 reduced the KM from 4.4 mM to 3.4 mM and increased the Vmax from 500 mOD/min to 588 mOD/min (FIGS. 33C and 33D).

The ability of the BIIB-9-1336/BIIB-12-917 and one-armed BIIB-9-1336 to increase the amidolytic activity to a similar level as the homodimeric, bivalent BIIB-9-1336 indicates that this activity results from the monovalent interaction between one BIIB-9-1336 arm and FIXa.

### Example 11

### Effect of BIIB-9-1336 on ATIII Inhibition of FIXa

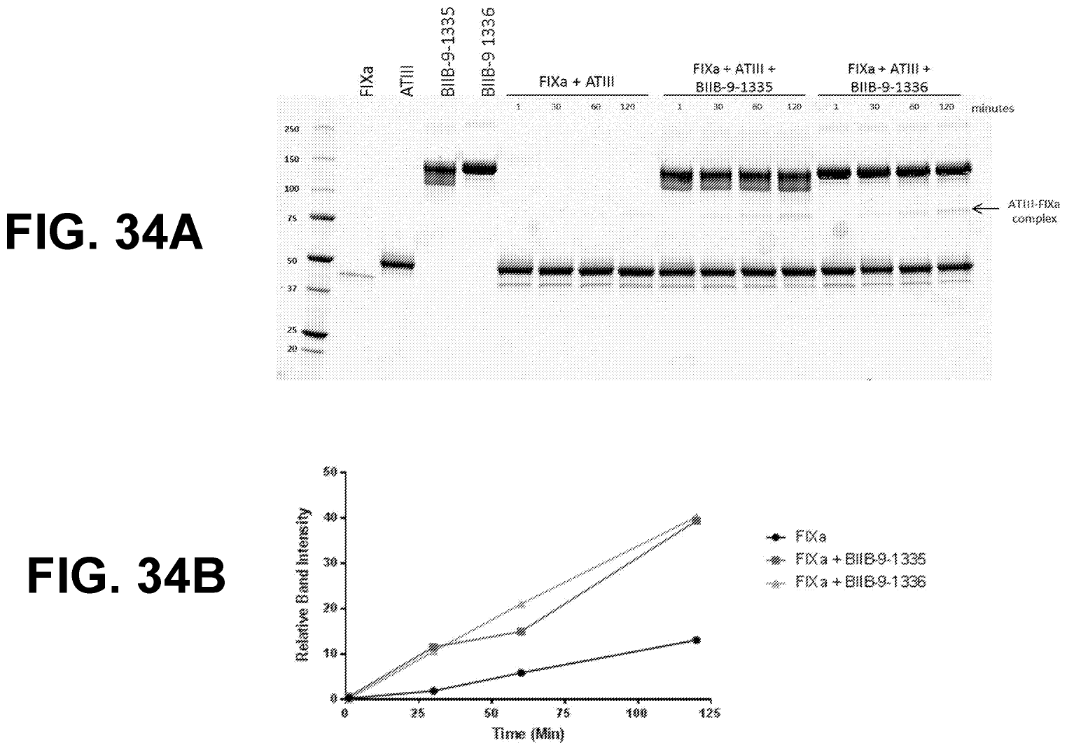

Anti-thrombin III is a serine protease inhibitor and leads to the formation of an irreversible bond between ATIII and the active site serine of FIXa. Hence, the mechanism of inhibition relies on the reactivity of the FIXa active site. Since BIIB-9-1336 is able to increase the amidolytic activity of FIXa, it stands to reason that it should also increase the rate of FIXa inhibition by ATIII. To test this, we incubated 500 nM of FIXa with 5000 nM ATIII in TBSCa in the precense of absence of 1500 nM BIIB-9-1336 or BIIB-9-1335, another anti-FIXa antibody that was identified during the affinity maturation of BIIB-9-484. Samples were removed at 1, 30, 60, and 120 minutes and mixed with non-reducing SDS loading buffer and run on a 4-20% BioRad stain-free gel (FIG. 34A).

The appearance of a band running near the 75 kDa molecular weight marker indicates formation of the ATIII-FIXa complex. Relative band intensity was quantified and plotted over time (FIG. 34B) and shows that BIIB-9-1335, and BIIB-9-1336 are able to increase the rate of ATIII inhibition of FIXa approximately 3-fold.

### Example 12

### Characterization of the BIIB-9-1336 Epitope on FIXa

To further our understanding of the interaction of BIIB-9-484 and BIIB-9-1336 with FIXa, we sought to determine the exact epitope of these antibodies. To do this, we first cloned, expressed, and purified only the Fab portion of each of these antibodies using standard methods. Subsequently, we mixed each Fab in a 1.5:1 molar ratio with FIXa in a calcium containing buffer and purified the resulting complex from excess Fab using size exclusion chromatography.

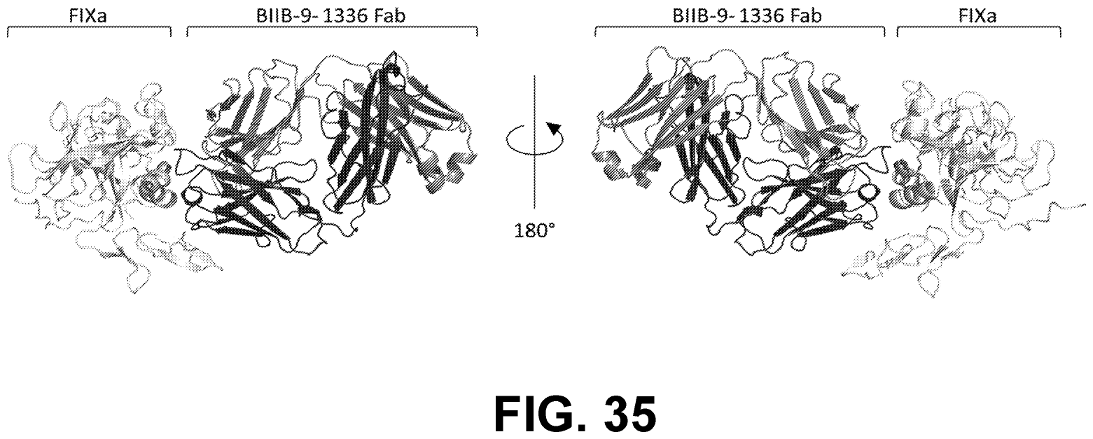

The resulting 1:1 complexes were screened in commercially available crystallization screens using the vapor diffusion method. While both complexes resulted in crystals, only the BIIB-9-1336 complex crystals produced high quality diffraction data. The resulting structure between the BIIB-9-1336 Fab and FIXa was solved by molecular replacement using standard methods and is shown in FIG. 35.

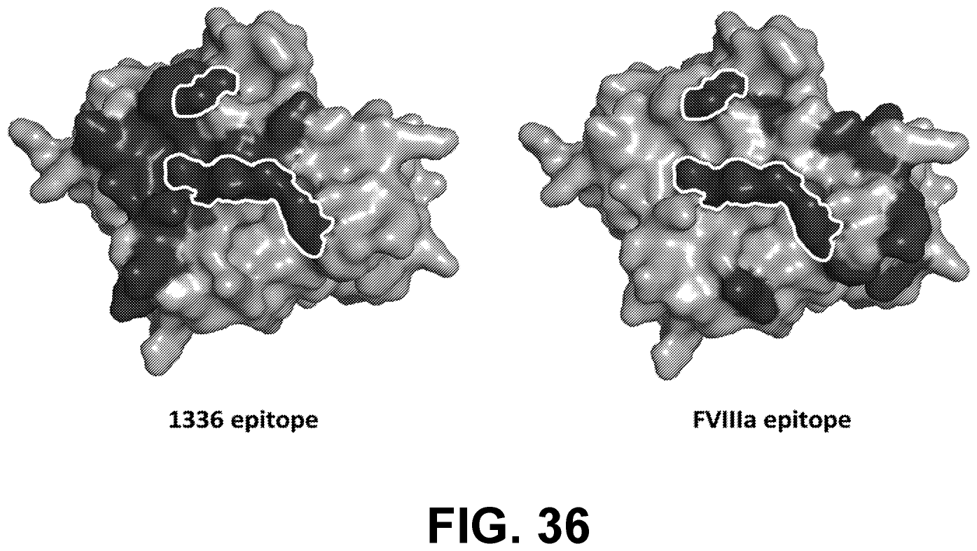

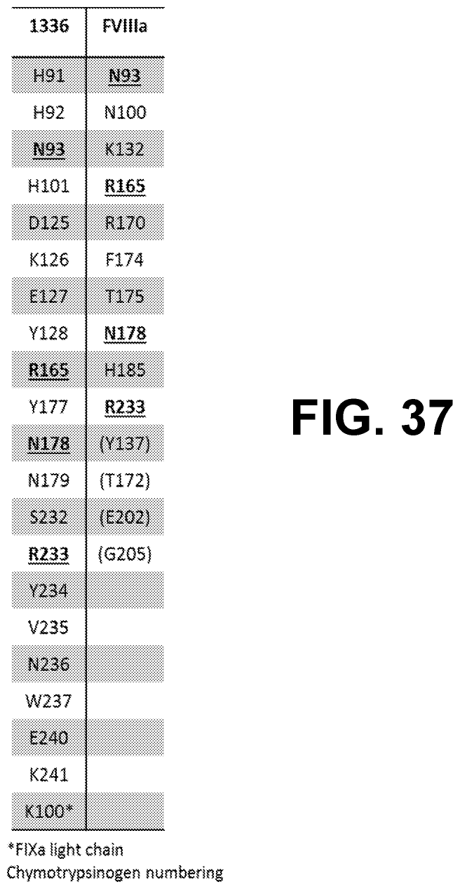

The residues on FIXa that constitute the BIIB-9-1336 epitope are shown in black in FIG. 36 and, for comparison, the residues that constitute the FVIIIa binding site on FIXa are also shown. These same residues are listed in the table in FIG. 37.

These data reveal that BIIB-9-1336 and FVIIIa share an overlapping epitope on FIXa. The residues that are shared are outlined in white in FIG. 36 and underlined and shown in bold in FIG. 37.

### Example 13

### Characterization of the BIIB-12-917 Epitope on FX

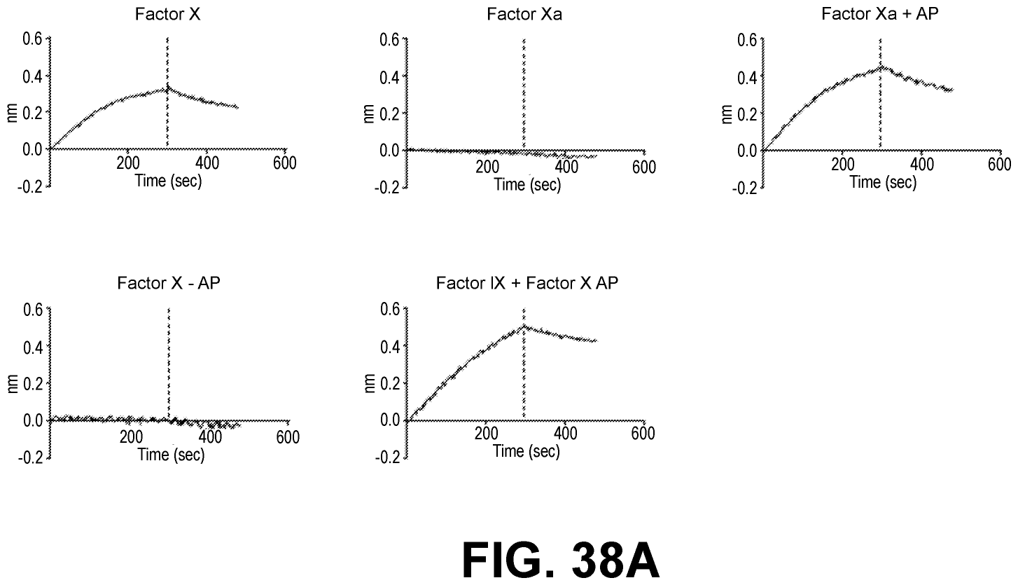

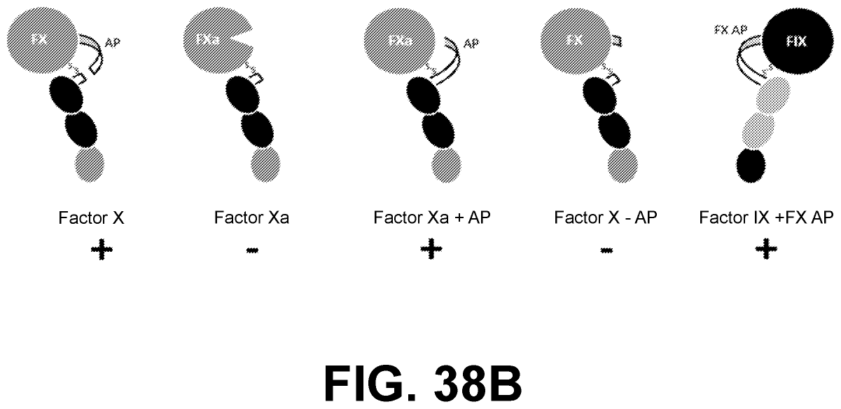

We sought to characterize the epitope of BIIB-12-917 on FX since it forms a productive bispecific antibody, with high FVIIIa-like activity, when paired with BIIB-9-484 or BIIB-9-1336. Using BLI, we tested the binding of BIIB-12-917 to a panel of different recombinant FX variants including wild type zymogen FX (FXz), wild type activated FX (FXa), activated FX which retains the activation peptide (FXa+AP), zymogen FX with the activation peptide lacking (FX-AP), and zymogen FIX which contains the activation peptide from FX (FIX+FX AP). A schematic representation of the variants is provided in FIG. 38B.

Using the Octet QK384 anti-Human IgG Quantitation (AHQ) dip and read were equilibrated in HBSCa for 60 seconds, followed by a 180 second loading step of the antibodies 15ug/mL onto the probes. After a 60 second baseline step in HBSCa, the antibody-loaded probes were exposed to 250 nM of each FX variant in HBSCa for 300 seconds, followed by a dissociation step in HBSCa for 180 seconds. The binding data in FIG. 38A show that BIIB-12-917 binds to all constructs containing the activation peptide of FX, but not to those lacking the activation peptide. Thus, the epitope of BIIB-12-917 lies within the activation peptide of FX.

### Example 14

### New Anti-FIXa Antibodies

A series of antibodies derived from BIIB-9-484, BIIB-9-1336, BIIB-9-578, or BIIB-9-619 was generated by introducing amino acid diversity into the CDR H1 and CDR H2; or CDR H3; or CDR L1, CDR L2 and CDR L3; or a combination of these. Antibodies with increased FIXa specificity and/or affinity were identified from these libraries using the methods described in Examples 1 and 5. The sequences of the VH and VL domains of these antibodies as well as their CDRs are provided below in Tables 6 and 7.

### Example 15

### Pharmacokinetics and Pharmacodynamics of Bispecific Antibodies in a Humanized Mouse Model of Hemophilia A

Pre-clinical pharmacokinetic assessment of bispecific antibodies is performed in a humanized mouse model of hemophilia A. Human FIX and human FX genes will be knocked-in, separately, to each respective mouse gene locus via homologous recombination using embryonic stem cell gene targeting. The FVIII gene of human FIX knock-in mice is subsequently edited by CRISPR/Cas9 technology to produce FVIII gene knock-outs. Breeding crosses between human FIX knock-ins and human FX knock-ins will result in mice homozygous for both human FIX and human FX (FIX-X-KI). This mouse model is useful for performing thrombosis models such as the inferior vena cava stasis model or the carotid artery ferric chloride-injury model. Breeding crosses between human FIX knock-ins lacking the mouse FVIII gene and human FX knock-ins will result in mice homozygous for human FIX, human FX, and deficient in FVIII (FIX-X-KI/FVIII-def). This mouse model is useful for performing hemostasis models such as the tail-clip model and the tail vein transection model.

In these experiments, FIX-X-KI/FVIII-def mice are dosed with a range of amounts of bispecific antibodies, control antibodies, rFVIII, and/or bypass therapies such as rFVIIa or activated prothrombin complex concentrates (aPCC). Groups of mice are dosed per molecule and per time point. At each time point post-dosing, mice are euthanized and blood is collected. A portion of the blood is used for rotational thromboelastometry (ROTEM) or other activity-based assay and the remaining blood is processed to plasma to determine circulating levels of bispecific antibodies by ELISA.

### Example 16

### Efficacy of Bispecific Antibodies in a Humanized Mouse Model of Hemophilia A

Acute efficacy of bispecific antibodies is determined with the tail-clip bleeding model in which the tail tip is amputated and total blood loss from FIX-X-KI/FVIII-def mice are dosed with a range of amounts of bispecific antibodies, control antibodies, rFVIII, and/or bypass therapies such as rFVIIa or aPCC, is measured as previously described (Dumont et al., 2012, Blood, 119(13):3024-3030). Prolonged efficacy of bispecific antibodies is determined with the tail vein transection model, as described previously (Pan et al., 2009, Blood, 114(13):2802-2811). Briefly, FIX-FX-KI/FVIII-def mice are dosed with a range of amounts of bispecific antibodies, control antibodies, rFVIII, and/or bypass therapies such as rFVIIa or aPCC and 24 hours post-dosing are anesthetized and subjected to transection of a tail vein. Bleeding time is recorded and mice are returned to cages for up to 24 hours at which point they are assessed for re-bleeding, overall responsiveness, activity and percent survival.

### Example 17

### Safety Assessment of Bispecific Antibodies in a Humanized Mouse Model

Thrombosis models are used to evaluate bispecific antibodies for potential prothrombotic properties. The inferior vena cava stasis model (Aleman et al., 2014, J Clin Invest, 124(8):3590-3600) or the carotid artery ferric chloride injury model (Machlus et al., 2011, Blood, 117(18):4953-4963) will be performed. Briefly, for the inferior vena cava model, FIX-FX-KI mice are anesthetized, dosed with a range of amounts of bispecific antibodies, control antibodies, rFVIII, and/or bypass therapies such as rFVIIa or aPCC and subjected to aseptic laparotomy to fully ligate the inferior vena cava. After 24 hours, mice are euthanized and thrombus weights are measured. For the carotid artery ferric chloride injury model, FIX-FX-KI mice are anesthetized and dosed with a range of amounts of bispecific antibodies, control antibodies, rFVIII, and/or bypass therapies such as rFVIIa or aPCC. The carotid artery is injured with a 10% ferric chloride solution. Changes in blood flow are monitored by Doppler ultrasound and the time to occlusion of the carotid artery is recorded.

### Example 18

### Pharmacokinetics and Pharmacodynamics of Bispecific Antibodies in Cynomolgus Monkeys

Pharmacokinetic and pharmacodynamic assessment of bispecific antibodies in cynomolgus monkeys is also performed, as described (Dumont et al., 2015, Thromb Res, 136(6):1266-1272). Briefly, an acquired hemophilia A model will be developed, such as described in Muto et al., 2014, Blood, 124(20):3165-3171. Hemophilic monkeys will be dosed with a range of amounts of bispecific antibodies, control antibodies, rFVIII, and/or bypass therapies such as rFVIIa or aPCC. Blood samples will be collected at a range of time points post-dose. A portion of the blood will be used for ROTEM analysis or other activity-based assay and the remaining blood will be processed to plasma to determine circulating levels of bispecific antibodies by ELISA.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

The contents of all cited references (including literature references, patents, patent applications, and websites) that may be cited throughout this application are hereby expressly incorporated by reference in their entirety for any purpose, as are the references cited therein.

TABLE 4
Sequences

| | | |
|---|---|---|
| SEQ | | |
| ID | | |
| NO | Description | Sequence |

Class I Antibodies - VH Sequences

| | | |
|---|---|---|
| 1 | BIIB-9-605_VH | EVQLLESGGGLVQPGGSLRLSCAASGFTFSSYAMSWVRQAPGKGLEWVSAISGSGGSTYYADSVKGRFTI |
| | Amino Acid | SRDNSKNTLYLQMNSLRAEDTAVYYCAKKRKYYGSHNPWGQGTLVTVSS |
| | Sequence | |
| 2 | BIIB-9-605_VH | GAGGTGCAGCTGTTGGAGTCTGGGGGAGGCTTGGTACAGCCTGGGGGGTCCCTGAGACTCTCCTGTGCAG |
| | Nucleic Acid | CCTCTGGATTCACCTTTAGCAGCTATGCCATGAGCTGGGTCCGCCAGGCTCCAGGGAAGGGGCTGGAGTG |
| | Sequence | GGTCTCAGCTATTAGTGGTAGTGGTGGTAGCACATACTACGCAGACTCCGTGAAGGGCCGGTTCACCATC |
| | | TCCAGAGACAATTCCAAGAACACGCTGTATCTGCAAATGAACAGCCTGAGAGCCGAGGACACGGCGGTGT |
| | | ACTACTGCGCTAAAAAGAGAAAGTACTACGGTTCACATAACCCATGGGGACAGGGTACATTGGTCACCGT |
| | | CTCCTCA |
| 3 | BIIB-9-475_VH | QVQLQESGPGLVKPSETLSLTCTVSGGSISSYYWSWIRQPPGKGLEWIGSIYYSGSTNYNPSLKSRVTIS |
| | Amino Acid | VDTSKNQFSLKLSSVTAADTAVYYCARDVGGYDYGVGAFDIWGQGTMVTVSS |
| | Sequence | |
| 4 | BIIB-9-475_VH | CAGGTGCAGCTGCAGGAGTCGGGCCCAGGACTGGTGAAGCCTTCGGAGACCCTGTCCCTCACCTGCACTG |
| | Nucleic Acid | TCTCTGGTGGCTCCATCAGTAGTTACTACTGGAGCTGGATCCGGCAGCCCCCAGGGAAGGGACTGGAGTG |
| | Sequence | GATTGGGTCAATCTATTACAGTGGGAGCACCAACTACAACCCCTCCCTCAAGAGTCGAGTCACCATATCA |
| | | GTAGACACGTCCAAGAACCAGTTCTCCCTGAAGCTGAGTTCTGTGACCGCCGCAGACACGGCGGTGTACT |
| | | ACTGCGCCAGAGATGTGGGCGGATACGACTACGGAGTGGGAGCCTTCGACATATGGGGTCAGGGTACAAT |
| | | GGTCACCGTCTCCTCA |
| 5 | BIIB-9-477_VH | EVQLLESGGGLVQPGGSLRLSCAASGFTFSNYAMSWVRQAPGKGLEWVSAISGSGGSTYYADSVKGRFTI |
| | Amino Acid | SRDNSKNTLYLQMNSLRAEDTAVYYCARGGVYSSSWMRFWGQGTLVTVSS |
| | Sequence | |
| 6 | BIIB-9-477_VH | GAGGTGCAGCTGTTGGAGTCTGGGGGAGGCTTGGTACAGCCTGGGGGGTCCCTGAGACTCTCCTGTGCAG |
| | Nucleic Acid | CCTCTGGATTCACCTTTAGCAATTATGCCATGAGCTGGGTCCGCCAGGCTCCAGGGAAGGGGCTGGAGTG |
| | Sequence | GGTCTCAGCTATTAGTGGTAGTGGTGGTAGCACATACTACGCAGACTCCGTGAAGGGCCGGTTCACCATC |
| | | TCCAGAGACAATTCCAAGAACACGCTGTATCTGCAAATGAACAGCCTGAGAGCCGAGGACACGGCGGTGT |
| | | ACTACTGCGCCAGAGGTGGCGTGTACAGCAGCTCGTGGATGAGATTCTGGGGACAGGGTACATTGGTCAC |
| | | CGTCTCCTCA |
| 7 | BIIB-9-479_VH | QVQLQESGPGLVKPSETLSLTCTVSGGSISSYYWSWIRQPPGKGLEWIGSIYYSGSTNYNPSLKSRVTIS |
| | Amino Acid | VDTSKNQFSLKLSSVTAADTAVYYCAREHYGDYPLFDIWGQGTMVTVSS |
| | Sequence | |
| 8 | BIIB-9-479_VH | CAGGTGCAGCTGCAGGAGTCGGGCCCAGGACTGGTGAAGCCTTCGGAGACCCTGTCCCTCACCTGCACTG |
| | Nucleic Acid | TCTCTGGTGGCTCCATCAGTAGTTACTACTGGAGCTGGATCCGGCAGCCCCCAGGGAAGGGACTGGAGTG |
| | Sequence | GATTGGGTCAATCTATTACAGTGGGAGCACCAACTACAACCCCTCCCTCAAGAGTCGAGTCACCATATCA |
| | | GTAGACACGTCCAAGAACCAGTTCTCCCTGAAGCTGAGTTCTGTGACCGCCGCAGACACGGCGGTGTACT |
| | | ACTGCGCCAGAGAGCACTACGGAGACTACCCACTATTCGACATATGGGGTCAGGGTACAATGGTCACCGT |
| | | CTCCTCA |
| 9 | BIIB-9-480_VH | QVQLQESGPGLVKPSETLSLTCAVSGYSISSGYYWGWIRQPPGKGLEWIGSSYHSGSTYYNPSLKSRVTI |
| | Amino Acid | SVDTSKNQFSLKLSSVTAADTAVYYCARDQQDYGAFDIWGQGTMVTVSS |
| | Sequence | |
| 10 | BIIB-9-480_VH | CAGGTGCAGCTGCAGGAGTCGGGCCCAGGACTGGTGAAGCCTTCGGAGACCCTGTCCCTCACCTGCGCTG |
| | Nucleic Acid | TCTCTGGTTACTCCATCAGCAGTGGTTACTACTGGGGCTGGATCCGGCAGCCCCCAGGGAAGGGGCTGGA |
| | Sequence | GTGGATTGGGAGTTCCTATCATAGTGGGAGCACCTACTACAACCCGTCCCTCAAGAGTCGAGTCACCATA |
| | | TCAGTAGACACGTCCAAGAACCAGTTCTCCCTGAAGCTGAGTTCTGTGACCGCCGCAGACACGGCGGTGT |
| | | ACTACTGCGCCAGGGACCAGCAAGACTACGGGGCCTTCGACATATGGGGTCAGGGTACAATGGTCACCGT |
| | | CTCCTCA |
| 11 | BIIB-9-558_VH | EVQLVESGGGLVKPGGSLRLSCAASGFTFSSYSMNWVRQAPGKGLEWVSSISSSSSYIYYADSVKGRFTI |
| | Amino Acid | SRDNAKNSLYLQMNSLRAEDTAVYYCARSYGYGYHDFDLWGRGTLVTVSS |
| | Sequence | |
| 12 | BIIB-9-558_VH | GAGGTGCAGCTGGTGGAGTCTGGGGGAGGCCTGGTCAAGCCTGGGGGGTCCCTGAGACTCTCCTGTGCAG |
| | Nucleic Acid | CCTCTGGATTCACCTTCAGTAGCTATAGCATGAACTGGGTCCGCCAGGCTCCAGGGAAGGGGCTGGAGTG |
| | Sequence | GGTCTCATCCATTAGTAGTAGTAGTAGTTACATATACTACGCAGACTCAGTGAAGGGCCGATTCACCATC |
| | | TCCAGAGACAACGCCAAGAACTCACTGTATCTGCAAATGAACAGCCTGAGAGCCGAGGACACGGCGGTGT |
| | | ACTACTGCGCCAGATCTTACGGATACGGATACCACGACTTCGACCTATGGGGGAGAGGTACCTTGGTCAC |
| | | CGTCTCCTCA |
| 13 | BIIB-9-414_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARDPYSYGMYYFDYWGQGTLVTVSS |
| | Sequence | |
| 14 | BIIB-9-414_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGACCCTTACTCCTACGGAATGTATTACTTTGATTACTGGGGACAGGGTACATTGGT |
| | | CACCGTCTCCTCA |
| 15 | BIIB-9-415_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARDGLSSGYYWDNWGQGTLVTVSS |
| | Sequence | |
| 16 | BIIB-9-415_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGATGGGTTGAGCAGCGGATACTACTGGGATAATTGGGGACAGGGTACATTGGTCAC |
| | | CGTCTCCTCA |
| 17 | BIIB-9-425_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCAREPTWDVAYDYWGQGTLVTVS |
| | Sequence | |
| 18 | BIIB-9-425_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGAGCCTACCTGGGACGTCGCCTACGATTATTGGGGACAGGGTACATTGGTCACCGT |
| | | CTCCTCA |
| 19 | BIIB-9-440_VH | QLQLQESGPGLVKPSETLSLTCTVSGGSISSSDYYWGWIRQPPGKGLEWIGSIYYSGSTYYNPSLKSRVT |
| | Amino Acid | ISVDTSKNQFSLKLSSVTAADTAVYYCARSPRHKVRGPNWFDPWGQGTLVTVSS |
| | Sequence | |
| 20 | BIIB-9-440_VH | CAGCTGCAGCTGCAGGAGTCGGGCCCAGGACTGGTGAAGCCTTCGGAGACCCTGTCCCTCACCTGCACTG |
| | Nucleic Acid | TCTCTGGTGGCTCCATCAGCAGTAGTGACTACTACTGGGGCTGGATCCGCCAGCCCCCAGGGAAGGGGCT |
| | Sequence | GGAGTGGATTGGGAGTATCTATTATAGTGGGAGCACCTACTACAACCCGTCCCTCAAGAGTCGAGTCACC |
| | | ATATCCGTAGACACGTCCAAGAACCAGTTCTCCCTGAAGCTGAGTTCTGTGACCGCCGCAGACACGGCGG |
| | | TGTACTACTGCGCCAGATCACCTAGGCACAAAGTGCGTGGCCCCAATTGGTTTGATCCATGGGGACAGGG |
| | | TACATTGGTCACCGTCTCCTCA |
| 21 | BIIB-9-452_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARDGRLSYTWDRWGQGTLVTVSS |
| | Sequence | |
| 22 | BIIB-9-452_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGACGGCAGACTAAGCTACACCTGGGACAGATGGGGACAGGGTACATTGGTCACCGT |
| | | CTCCTCA |
| 23 | BIIB-9-460_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARDISTDGESSLYYYMDVWGKGTTVTVSS |
| | Sequence | |
| 24 | BIIB-9-460_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCAAGGGATATTTCTACCGACGGGGAATCATCACTTTACTACTACATGGACGTATGGGGCAA |
| | | GGGTACAACTGTCACCGTCTCCTCA |
| 25 | BIIB-9-461_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARGPTDSSGYLDMDVWGKGTTVTVSS |
| | Sequence | |
| 26 | BIIB-9-461_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGGACCCACTGACAGCAGCGGATACTTGGACATGGACGTATGGGGCAAGGGTACAAC |
| | | TGTCACCGTCTCCTCA |
| 27 | BIIB-9-465_VH | QVQLVQSGAEVKKPGASVKVSCKASGYTFTSYYMVWVRQAPGQGLEWMGIINPSGGSTSYAQKFQGRVTM |
| | Amino Acid | TRDTSTSTVYMELSSLRSEDTAVYYCARSPTDGYYFDLWGRGTLVTVSS |
| | Sequence | |
| 28 | BIIB-9-465_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGGTCTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGATCACCTACGGACGGATACTATTTCGACCTATGGGGGAGAGGTACCTTGGTCACCGT |
| | | CTCCTCA |
| 29 | BIB-4-564_VH | QVQLQESGPGLVKPSETLSLTCAVSGYSISSGYYWAWIRQPPGKGLEWIGSIYHSGSTYYNPSLKSRVTI |
| | Amino Acid | SVDTSKNQFSLKLSSVTAADTAVYYCARDPGYSWEYFDYWGQGTLVTVSS |
| | Sequence | |
| 30 | BIIB-9-564_VH | CAGGTGCAGCTGCAGGAGTCGGGCCCAGGACTGGTGAAGCCTTCGGAGACCCTGTCCCTCACCTGCGCTG |
| | Nucleic Acid | TCTCTGGTTACTCCATCAGCAGTGGTTACTACTGGGCTTGGATCCGGCAGCCCCCAGGGAAGGGGCTGGA |
| | Sequence | GTGGATTGGGAGTATCTATCATAGTGGGAGCACCTACTACAACCCGTCCCTCAAGAGTCGAGTCACCATA |
| | | TCAGTAGACACGTCCAAGAACCAGTTCTCCCTGAAGCTGAGTTCTGTGACCGCCGCAGACACGGCGGTGT |
| | | ACTACTGCGCCAGAGATCCAGGATACAGCTGGGAGTACTTTGACTACTGGGGACAGGGTACATTGGTCAC |
| | | CGTCTCCTCA |
| 31 | BIIB-9-484_VH | EVQLVESGGGLVKPGGSLRLSCAASGFTFSSYSMNWVRQAPGKGLEWVSSISSSSSYIYYADSVKGRFTI |
| | Amino Acid | SRDNAKNSLYLQMNSLRAEDTAVYYCARDVGGYAGYYGMDVWGQGTTVTVSS |
| | Sequence | |
| 32 | BIIB-9-484_VH | GAGGTGCAGCTGGTGGAGTCTGGGGGAGGCCTGGTCAAGCCTGGGGGGTCCCTGAGACTCTCCTGTGCAG |
| | Nucleic Acid | CCTCTGGATTCACCTTCAGTAGCTATAGCATGAACTGGGTCCGCCAGGCTCCAGGGAAGGGGCTGGAGTG |
| | Sequence | GGTCTCATCCATTAGTAGTAGTAGTAGTTACATATACTACGCAGACTCAGTGAAGGGCCGATTCACCATC |
| | | TCCAGAGACAACGCCAAGAACTCACTGTATCTGCAAATGAACAGCCTGAGAGCCGAGGACACGGCGGTGT |
| | | ACTACTGCGCCAGAGATGTAGGAGGATACGCAGGGTACTACGGCATGGATGTATGGGGCCAGGGAACAAC |
| | | TGTCACCGTCTCCTCA |
| 33 | BIIB-9-469_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARDLGYGRSYDFDLWGRGTLVTVSS |
| | Sequence | |
| 34 | BIIB-9-469_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGACTTGGGATACGGCAGAAGTTATGACTTCGACCTATGGGGGAGAGGTACCTTGGT |
| | | CACCGTCTCCTCA |
| 35 | BIIB-9-566_VH | QVQLVQSGAEVKKPGASVKVSCKASGYTFTSYYMVWVRQAPGQGLEWMGIINPSGGSTSYAQKFQGRVTM |
| | Amino Acid | TRDTSTSTVYMELSSLRSEDTAVYYCARVPTYRYSYLAFDIWGQGTMVTVSS |
| | Sequence | |
| 36 | BIIB-9-566_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGGTCTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGTCCCTACATACAGATACAGCTACTTAGCCTTCGATATCTGGGGTCAGGGTACAAT |
| | | GGTCACCGTCTCCTCA |
| 37 | BIIB-9-567_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARLGRRYYAYDGMDVWGQGTTVTVSS |
| | Sequence | |
| 38 | BIIB-9-567_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGATTGGGAAGAAGGTACTACGCCTATGATGGGATGGATGTTTGGGGCCAGGGAACAAC |
| | | TGTCACCGTCTCCTCA |
| 39 | BIIB-9-569_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARDGSGYSPYSFDPWGQGTLVTVSS |
| | Sequence | |
| 40 | BIIB-9-569_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGACGGATCTGGATACAGTCCATACAGCTTCGACCCATGGGGACAGGGTACATTGGT |
| | | CACCGTCTCCTCA |
| 41 | BIIB-9-588_VH | QVQLVQSGAEVKKPGASVKVSCKASGYTFTSYYMHWVRQAPGQGLEWMGIINPSGGSTTYAQKFQGRVTM |
| | Amino Acid | TRDTSTSTVYMELSSLRSEDTAVYYCARDGGGSYDYWSGYWYDVWGQGTTVTVSS |
| | Sequence | |
| 42 | BIIB-9-588_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAACCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGATGGTGGCGGATCCTACGACTACTGGAGCGGATACTGGTACGACGTATGGGGTCA |
| | | GGGTACAACTGTCACCGTCTCCTCA |
| 43 | BIIB-9-611_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCAREVISRVSYFDLWGRGTLVTVSS |
| | Sequence | |
| 44 | BIIB-9-611_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGAGGTGATATCCAGGGTTAGCTACTTCGACCTATGGGGGAGAGGTACCTTGGTCAC |
| | | CGTCTCCTCA |
| 45 | BIIB-9-619_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARDGPRVSDYYMDVWGKGTTVTVSS |
| | Sequence | |
| 46 | BIIB-9-619_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGACGGACCAAGAGTCAGTGACTACTACATGGACGTATGGGGCAAGGGTACAACTGT |
| | | CACCGTCTCCTCA |
| 47 | BIIB-9-626_VH | QVQLVQSGAEVKKPGASVKVSCKASGYTFTSYYMHWVRQAPGQGLEWMGVINPSGGSTSYAQKFQGRVTM |
| | Amino Acid | TRDTSTSTVYMELSSLRSEDTAVYYCARDLQYSMTYFDYWGQGTLVTVSS |
| | Sequence | |
| 48 | BIIB-9-626_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | TGATGGGAGTCATCAACCCTAGTGGGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGACTTGCAGTATAGCATGACATACTTCGACTACTGGGGACAGGGTACATTGGTCAC |
| | | CGTCTCCTCA |
| 49 | BIIB-9-883_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARDGRLSYTWDRWGQGTLVTVSS |
| | Sequence | |
| 50 | BIIB-9-883_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGACGGCAGACTAAGCTACACCTGGGACAGATGGGGACAGGGTACATTGGTCACCGT |
| | | CTCCTCA |
| 51 | BIIB-9-419_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCAREPTFYASYFDLWGRGTLVTVS |
| | Sequence | |
| 52 | BIIB-9-419_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGAGCCTACCTTCTACGCCAGCTACTTCGACCTATGGGGGAGAGGTACCTTGGTCAC |
| | | CGTCTCCTCA |
| 53 | BIIB-9-451_VH | QLQLQESGPGLVKPSETLSLTCTVSGGSISSSSYYWGWIRQPPGKGLEWIGSISYSGSTYYNPSLKSRVT |
| | Amino Acid | ISVDTSKNQFSLKLSSVTAADTAVYYCARDSGGYYYQGFDYWGQGTLVTVSS |
| | Sequence | |
| 54 | BIIB-9-451_VH | CAGCTGCAGCTGCAGGAGTCGGGCCCAGGACTGGTGAAGCCTTCGGAGACCCTGTCCCTCACCTGCACTG |
| | Nucleic Acid | TCTCTGGTGGCTCCATCAGCAGTAGTAGTTACTACTGGGGCTGGATCCGCCAGCCCCCAGGGAAGGGGCT |
| | Sequence | GGAGTGGATTGGGAGTATCTCCTATAGTGGGAGCACCTACTACAACCCGTCCCTCAAGAGTCGAGTCACC |
| | | ATATCCGTAGACACGTCCAAGAACCAGTTCTCCCTGAAGCTGAGTTCTGTGACCGCCGCAGACACGGCGG |
| | | TGTACTACTGCGCCAGAGACTCAGGAGGATACTACTACCAGGGATTCGATTACTGGGGACAGGGTACATT |
| | | GGTCACCGTCTCCTCA |
| 55 | BIIB-9-473_VH | VQLLESGGGLVQPGGSLRLSCAASGFTFSSYAMSWVRQAPGKGLEWVSAISGSGGSTYYADSVKGRFTIS |
| | Amino Acid | RDNSKNTLYLQMNSLRAEDTAVYYCAKDRLRYSRWYDGMDVWGQGTTVTVSS |
| | Sequence | |
| 56 | BIIB-9-473_VH | GAGGTGCAGCTGTTGGAGTCTGGGGGAGGCTTGGTACAGCCTGGGGGGTCCCTGAGACTCTCCTGTGCAG |
| | Nucleic Acid | CCTCTGGATTCACCTTTAGCAGCTATGCCATGAGCTGGGTCCGCCAGGCTCCAGGGAAGGGGCTGGAGTG |
| | Sequence | GGTCTCAGCTATTAGTGGTAGTGGTGGTAGCACATACTACGCAGACTCCGTGAAGGGCCGGTTCACCATC |
| | | TCCAGAGACAATTCCAAGAACACGCTGTATCTGCAAATGAACAGCCTGAGAGCCGAGGACACGGCGGTGT |
| | | ACTACTGCGCAAAAGACAGGTTGAGATACAGCAGATGGTACGATGGGATGGATGTTTGGGGCCAGGGAAC |
| | | AACTGTCACCGTCTCCTCA |
| 57 | BIIB-9-565_VH | QLQLQESGPGLVKPSETLSLTCTVSGGSISSSSYYWGWIRQPPGKGLEWIGSIYYSGSTYYNPSLKSRVT |
| | Amino Acid | ISVDTSKNQFSLKLSSVTAADTAVYYCARAGMYSSYANWFDPWGQGTLVTVSS |
| | Sequence | |
| 58 | BIIB-9-565_VH | CAGCTGCAGCTGCAGGAGTCGGGCCCAGGACTGGTGAAGCCTTCGGAGACCCTGTCCCTCACCTGCACTG |
| | Nucleic Acid | TCTCTGGTGGCTCCATCAGCAGTAGTAGTTACTACTGGGGCTGGATCCGCCAGCCCCCAGGGAAGGGGCT |
| | Sequence | GGAGTGGATTGGGAGTATCTATTATAGTGGGAGCACCTACTACAACCCGTCCCTCAAGAGTCGAGTCACC |
| | | ATATCCGTAGACACGTCCAAGAACCAGTTCTCCCTGAAGCTGAGTTCTGTGACCGCCGCAGACACGGCGG |
| | | TGTACTACTGCGCCAGAGCTGGAATGTACTCCAGTTATGCTAACTGGTTTGACCCATGGGGACAGGGTAC |
| | | ATTGGTCACCGTCTCCTC |
| 59 | BIIB-9-573_VH | QLQLQESGPGLVKPSETLSLTCTVSGGSISSSSYYWGWIRQPPGKGLEWIGSIYYSGSTYYNPSLKSRVT |
| | Amino Acid | ISVDTSKNQFSLKLSSVTAADTAVYYCARESKTKGYLDLWGRGTLVTVSS |
| | Sequence | |
| 60 | BIIB-9-573_VH | CAGCTGCAGCTGCAGGAGTCGGGCCCAGGACTGGTGAAGCCTTCGGAGACCCTGTCCCTCACCTGCACTG |
| | Nucleic Acid | TCTCTGGTGGCTCCATCAGCAGTAGTAGTTACTACTGGGGCTGGATCCGCCAGCCCCCAGGGAAGGGGCT |
| | Sequence | GGAGTGGATTGGGAGTATCTATTATAGTGGGAGCACCTACTACAACCCGTCCCTCAAGAGTCGAGTCACC |
| | | ATATCCGTAGACACGTCCAAGAACCAGTTCTCCCTGAAGCTGAGTTCTGTGACCGCCGCAGACACGGCGG |
| | | TGTACTACTGCGCCAGAGAATCTAAGACCAAAGGGTACTTAGACCTATGGGGGAGAGGTACCTTGGTCAC |
| | | CGTCTCCTCA |
| 61 | BIIB-9-579_VH | QVQLVQSGAEVKKPGASVKVSCKASGYTFTSYYMVWVRQAPGQGLEWMGIINPSGGSTSYAQKFQGRVTM |
| | Amino Acid | TRDTSTSTVYMELSSLRSEDTAVYYCARGPWYSYYYMDVWGKGTTVTVSS |
| | Sequence | |
| 62 | BIIB-9-579_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGGTCTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGGGCCTTGGTACAGTTATTACTACATGGACGTATGGGGCAAGGGTACAACTGTCAC |
| | | CGTCTCCTCA |
| 63 | BIIB-9-581_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARDPLYYREGYVFDYWGQGTLVTVSS |
| | Sequence | |
| 64 | BIIB-9-581_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGATCCTTTGTACTACAGAGAAGGATACGTTTTCGATTACTGGGGACAGGGTACATT |
| | | GGTCACCGTCTCCTCA |
| 65 | BIIB-9-582_VH | QVQLVQSGAEVKKPGASVKVSCKASGYTFTSYYMVWVRQAPGQGLEWMGIINPSGGSTSYAQKFQGRVTM |
| | Amino Acid | TRDTSTSTVYMELSSLRSEDTAVYYCARAPTYFYSYGMDVWGQGTTVTVSS |
| | Sequence | |
| 66 | BIIB-9-582_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGGTCTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGCCCCTACATACTTCTACAGCTACGGAATGGACGTATGGGGCCAGGGAACAACTGT |
| | | CACCGTCTCCTCA |
| 67 | BIIB-9-585_VH | QLQLQESGPGLVKPSETLSLTCTVSGGSISSSDYYWGWIRQPPGKGLEWIGSIYYSGSTYYNPSLKSRVT |
| | Amino Acid | ISVDTSKNQFSLKLSSVTAADTAVYYCAREVGTYYYGLDFWFDPWGQGTLVTVSS |
| | Sequence | |
| 68 | BIIB-9-585_VH | CAGCTGCAGCTGCAGGAGTCGGGCCCAGGACTGGTGAAGCCTTCGGAGACCCTGTCCCTCACCTGCACTG |
| | Nucleic Acid | TCTCTGGTGGCTCCATCAGCAGTAGTGACTACTACTGGGGCTGGATCCGCCAGCCCCCAGGGAAGGGGCT |
| | Sequence | GGAGTGGATTGGGAGTATCTATTATAGTGGGAGCACCTACTACAACCCGTCCCTCAAGAGTCGAGTCACC |
| | | ATATCCGTAGACACGTCCAAGAACCAGTTCTCCCTGAAGCTGAGTTCTGTGACCGCCGCAGACACGGCGG |
| | | TGTACTACTGCGCCAGAGAAGTCGGAACATACTACTACGGCTTAGACTTCTGGTTTGACCCCTGGGGACA |
| | | GGGTACATTGGTCACCGTCTCCTCA |
| 69 | BIIB-9-587_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARGPPGYEYYMDVWGKGTTVTVSS |
| | Sequence | |
| 70 | BIIB-9-587_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGGACCGCCTGGATACGAGTACTACATGGACGTATGGGGCAAGGGAACAACTGTCAC |
| | | CGTCTCCTCA |
| 71 | BIIB-9-590_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARDQEWAYYGMDVWGQGTTVTVSS |
| | Sequence | |
| 72 | BIIB-9-590_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGACCAGGAGTGGGCCTACTACGGCATGGACGTATGGGGCCAGGGAACAACTGTCAC |
| | | CGTCTCCTCA |
| 73 | BIIB-9-592_VH | QLQLQESGPGLVKPSETLSLTCTVSGGSISSNSYYWGWIRQPPGKGLEWIGSIYYSGSTYYNPSLKSRVT |
| | Amino Acid | ISVDTSKNQFSLKLSSVTAADTAVYYCARDSNYDSSGYALYYYGMDVWGQGTTVTVSS |
| | Sequence | |
| 74 | BIIB-9-592_VH | CAGCTGCAGCTGCAGGAGTCGGGCCCAGGACTGGTGAAGCCTTCGGAGACCCTGTCCCTCACCTGCACTG |
| | Nucleic Acid | TCTCTGGTGGCTCCATCAGCAGTAACAGTTACTACTGGGGCTGGATCCGCCAGCCCCCAGGGAAGGGGCT |
| | Sequence | GGAGTGGATTGGGAGTATCTATTATAGTGGGAGCACCTACTACAACCCGTCCCTCAAGAGTCGAGTCACC |
| | | ATATCCGTAGACACGTCCAAGAACCAGTTCTCCCTGAAGCTGAGTTCTGTGACCGCCGCAGACACGGCGG |
| | | TGTACTACTGCGCCAGAGACTCTAACTACGACAGCAGCGGATACGCCTTATACTACTATGGGATGGATGT |
| | | ATGGGGCCAGGGAACAACTGTCACCGTCTCCTCA |
| 75 | BIIB-9-606_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARTKWSSSPYGMDVWGQGTTVTVSS |
| | Sequence | |
| 76 | BIIB-9-606_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAACTAAATGGTCATCAAGCCCATACGGAATGGACGTATGGGGCCAGGGAACAACTGT |
| | | CACCGTCTCCTCA |
| 77 | BIIB-9-608_VH | QLQLQESGPGLVKPSETLSLTCTVSGGSISSSSYYWGWIRQPPGKGLEWIGSIYYSGSTYYNPSLKSRVT |
| | Amino Acid | ISVDTSKNQFSLKLSSVTAADTAVYYCARDRLETSEAGMDVWGQGTTVTVSS |
| | Sequence | |
| 78 | BIIB-9-608_VH | CAGCTGCAGCTGCAGGAGTCGGGCCCAGGACTGGTGAAGCCTTCGGAGACCCTGTCCCTCACCTGCACTG |
| | Nucleic Acid | TCTCTGGTGGCTCCATCAGCAGTAGTAGTTACTACTGGGGCTGGATCCGCCAGCCCCCAGGGAAGGGGCT |
| | Sequence | GGAGTGGATTGGGAGTATCTATTATAGTGGGAGCACCTACTACAACCCGTCCCTCAAGAGTCGAGTCACC |
| | | ATATCCGTAGACACGTCCAAGAACCAGTTCTCCCTGAAGCTGAGTTCTGTGACCGCCGCAGACACGGCGG |
| | | TGTACTACTGCGCCAGAGACAGGTTGGAAACAAGCGAGGCAGGAATGGACGTATGGGGCCAGGGAACAAC |
| | | TGTCACCGTCTCCTCA |
| 79 | BIIB-9-616_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARGGSKYFFDLWGRGTLVTVSS |
| | Sequence | |
| 80 | BIIB-9-616_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGGCGGTTCTAAATACTTCTTCGACCTATGGGGGAGAGGTACCTTGGTCACCGTCTC |
| | | CTCA |
| 81 | BIIB-9-621_VH | QLQLQESGPGLVKPSETLSLTCTVSGGSISSSSYYWGWIRQPPGKGLEWIGSIYYSGSTYYNPSLKSRVT |
| | Amino Acid | ISVDTSKNQFSLKLSSVTAADTAVYYCARDGSISGSRFDYWGQGTLVTVSS |
| | Sequence | |
| 82 | BIIB-9-621_VH | CAGCTGCAGCTGCAGGAGTCGGGCCCAGGACTGGTGAAGCCTTCGGAGACCCTGTCCCTCACCTGCACTG |
| | Nucleic Acid | TCTCTGGTGGCTCCATCAGCAGTAGTAGTTACTACTGGGGCTGGATCCGCCAGCCCCCAGGGAAGGGGCT |
| | Sequence | GGAGTGGATTGGGAGTATCTATTATAGTGGGAGCACCTACTACAACCCGTCCCTCAAGAGTCGAGTCACC |
| | | ATATCCGTAGACACGTCCAAGAACCAGTTCTCCCTGAAGCTGAGTTCTGTGACCGCCGCAGACACGGCGG |
| | | TGTACTACTGCGCCAGAGACGGATCTATATCCGGATCTAGATTCGACTACTGGGGACAGGGTACATTGGT |
| | | CACCGTCTCCTCA |
| 83 | BIIB-9-622_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARDGATTVSYLSFDIWGQGTMVTVSS |
| | Sequence | |
| 84 | BIIB-9-622_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGATGGCGCTACCACAGTAAGCTATTTGTCATTCGACATATGGGGTCAGGGTACAAT |
| | | GGTCACCGTCTCCTCA |
| 85 | BIIB-9-627_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARADYDYWSGYGGLGMDVWGQGTTVTVSS |
| | Sequence | |
| 86 | BIIB-9-627_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGCTGACTACGACTACTGGAGCGGATACGGAGGTCTCGGAATGGACGTATGGGGCCA |
| | | GGGAACAACTGTCACCGTCTCCTCA |
| 87 | BIIB-9-1335_VH | EVQLVESGGGLVKPGGSLRLSCAASGFTFSSYDMHWVRQAPGKGLEWVSSISSGSSYIYYADSVKGRFTI |
| | Amino Acid | SRDNAKNSLYLQMNSLRAEDTAVYYCARDVGGYAGYYGMDVWGQGTTVTVSS |
| | Sequence | |
| 88 | BIIB-9-1335_VH | GAGGTGCAGCTGGTGGAGTCTGGGGGAGGCCTGGTCAAGCCTGGGGGGTCCCTGAGACTCTCCTGTGCAG |
| | Nucleic Acid | CCTCTGGATTCACCTTCAGTAGCTATGATATGCATTGGGTCCGCCAGGCTCCAGGGAAGGGGCTGGAGTG |
| | Sequence | GGTCTCATCCATTAGTAGTGGTAGTAGTTACATATACTACGCAGACTCAGTGAAGGGCCGATTCACCATC |
| | | TCCAGAGACAACGCCAAGAACTCACTGTATCTGCAAATGAACAGCCTGAGAGCCGAGGACACGGCGGTGT |
| | | ACTACTGCGCCAGAGATGTAGGAGGATACGCAGGGTACTACGGCATGGATGTATGGGGCCAGGGAACAAC |
| | | TGTCACCGTCTCCTCA |
| 89 | BIIB-9-1336_VH | EVQLVESGGGLVKPGGSLRLSCAASGFTFGSYDMNWVRQAPGKGLEWVSSISSGESYIYYAESVKGRFTI |
| | Amino Acid | SRDNAKNSLYLQMNSLRAEDTAVYYCARDVGGYAGYYGMDVWGQGTTVTVSS |
| | Sequence | |
| 90 | BIIB-9-1336_VH | GAGGTGCAGCTGGTGGAGTCTGGGGGAGGCCTGGTCAAGCCTGGGGGGTCCCTGAGACTCTCCTGTGCAG |
| | Nucleic Acid | CCTCTGGATTCACCTTCGGGAGCTATGATATGAACTGGGTCCGCCAGGCTCCAGGGAAGGGGCTGGAGTG |
| | Sequence | GGTCTCATCCATTAGTAGTGGTGAGAGTTACATATACTACGCAGAGTCAGTGAAGGGCCGATTCACCATC |
| | | TCCAGAGACAACGCCAAGAACTCACTGTATCTGCAAATGAACAGCCTGAGAGCCGAGGACACGGCGGTGT |
| | | ACTACTGCGCCAGAGATGTAGGAGGATACGCAGGGTACTACGGCATGGATGTATGGGGCCAGGGAACAAC |
| | | TGTCACCGTCTCCTCA |

Class II Antibodies - VH Sequences

| | | |
|---|---|---|
| 91 | BIIB-9-408_VH | QVQLVQSGAEVKKPGASVKVSCKASGYTFTSYYMHWVRQAPGQGLEWMGIINPSGGSTTYAQKFQGRVTM |
| | Amino Acid | TRDTSTSTVYMELSSLRSEDTAVYYCARDPGAYDDWSGYDDYGMDVWGQGTTVTVSS |
| | Sequence | |
| 92 | BIIB-9-408_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAACCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGATCCAGGAGCCTACGACGACTGGAGCGGATATGATGATTATGGAATGGACGTATG |
| | | GGGCCAGGGAACAACTGTCACCGTCTCCTCA |
| 93 | BIIB-9-416_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCAREGPMLDYPTYSNWFDPWGQGTLVTVS |
| | Sequence | |
| 94 | BIIB-9-416_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGAAGGTCCTATGCTAGACTACCCAACCTACAGCAACTGGTTCGACCCATGGGGACA |
| | | GGGTACATTGGTCACCGTCTCCTCA |
| 95 | BIIB-9-629_VH | QVQLVQSGAEVKKPGASVKVSCKASGYTFTSYYMHWVRQAPGQGLEWMGVINPSGGSTSYAQKFQGRVTM |
| | Amino Acid | TRDTSTSTVYMELSSLRSEDTAVYYCARDPSQDYATGTGWFDPWGQGTLVTVSS |
| | Sequence | |
| 96 | BIIB-9-629_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAGTCATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGATCCCTCTCAAGACTACGCAACCGGAACCGGTTGGTTCGATCCCTGGGGACAGGG |
| | | TACATTGGTCACCGTCTCCTCA |
| 97 | BIIB-9-885_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCAREGPMLDYPTYSNWFDPWGQGTLVTVSS |
| | Sequence | |
| 98 | BIIB-9-885_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGAAGGTCCTATGCTAGACTACCCAACCTACAGCAACTGGTTCGACCCATGGGGACA |
| | | GGGTACATTGGTCACCGTCTCCTCA |

Class III Antibodies - VH Sequences

| | | |
|---|---|---|
| 99 | BIIB-9-607_VH | QLQLQESGPGLVKPSETLSLTCTVSGGSISSSSYYWGWIRQPPGKGLEWIGSISYSGSTYYNPSLKSRVT |
| | Amino Acid | ISVDTSKNQFSLKLSSVTAADTAVYYCARDRGYSYEDFDLWGRGTLVTVS |
| | Sequence | |
| 100 | BIIB-9-607_VH | CAGCTGCAGCTGCAGGAGTCGGGCCCAGGACTGGTGAAGCCTTCGGAGACCCTGTCCCTCACCTGCACTG |
| | Nucleic Acid | TCTCTGGTGGCTCCATCAGCAGTAGTAGTTACTACTGGGGCTGGATCCGCCAGCCCCCAGGGAAGGGGCT |
| | Sequence | GGAGTGGATTGGGAGTATCTCCTATAGTGGGAGCACCTACTACAACCCGTCCCTCAAGAGTCGAGTCACC |
| | | ATATCCGTAGACACGTCCAAGAACCAGTTCTCCCTGAAGCTGAGTTCTGTGACCGCCGCAGACACGGCGG |
| | | TGTACTACTGCGCCAGAGATCGTGGATACAGCTACGAGGACTTCGACCTATGGGGGAGAGGTACCTTGGT |
| | | CACCGTCTCCTCA |
| 101 | BIIB-9-471_VH | QVQLQESGPGLVKPSQTLSLTCTVSGGSISSGGYYWSWIRQHPGKGLEWIGYIYYSGSTYYNPSLKSRVT |
| | Amino Acid | ISVDTSKNQFSLKLSSVTAADTAVYYCARSGVSGSGSDNWFDPWGQGTTVTVSS |
| | Sequence | |
| 102 | BIIB-9-471_VH | CAGGTGCAGCTGCAGGAGTCGGGCCCAGGACTGGTGAAGCCTTCACAGACCCTGTCCCTCACCTGTACTG |
| | Nucleic Acid | TCTCTGGTGGCTCCATCAGCAGTGGTGGTTACTACTGGAGCTGGATCCGCCAGCACCCAGGGAAGGGCCT |
| | Sequence | GGAGTGGATTGGGTACATCTATTACAGTGGGAGCACCTACTACAACCCGTCCCTCAAGAGTCGAGTTACC |
| | | ATATCAGTAGACACGTCTAAGAACCAGTTCTCCCTGAAGCTGAGTTCTGTGACCGCCGCAGACACGGCGG |
| | | TGTACTACTGCGCCAGATCAGGAGTGAGCGGATCGGGATCTGATAATTGGTTCGATCCATGGGGACAGGG |
| | | TACAACTGTCACCGTCTCCTCA |
| 103 | BIIB-9-472_VH | EVQLVESGGGLVKPGGSLRLSCAASGFTFSSYSMNWVRQAPGKGLEWVSSISSSSSYIYYADSVKGRFTI |
| | Amino Acid | SRDNAKNSLYLQMNSLRAEDTAVYYCARGGRYSGSWSWNIWGQGTMVTVSS |
| | Sequence | |
| 104 | BIIB-9-472_VH | GAGGTGCAGCTGGTGGAGTCTGGGGGAGGCCTGGTCAAGCCTGGGGGGTCCCTGAGACTCTCCTGTGCAG |
| | Nucleic Acid | CCTCTGGATTCACCTTCAGTAGCTATAGCATGAACTGGGTCCGCCAGGCTCCAGGGAAGGGGCTGGAGTG |
| | Sequence | GGTCTCATCCATTAGTAGTAGTAGTAGTTACATATACTACGCAGACTCAGTGAAGGGCCGATTCACCATC |
| | | TCCAGAGACAACGCCAAGAACTCACTGTATCTGCAAATGAACAGCCTGAGAGCCGAGGACACGGCGGTGT |
| | | ACTACTGCGCCAGAGGAGGTAGATACAGCGGCTCGTGGAGCTGGAACATATGGGGTCAGGGTACAATGGT |
| | | CACCGTCTCCTCA |
| 105 | BIIB-9-439_VH | QVQLVQSGAEVKKPGASVKVSCKASGYTFTSYYMHWVRQAPGQGLEWMGIINPSGGSTTYAQKFQGRVTM |
| | Amino Acid | TRDTSTSTVYMELSSLRSEDTAVYYCAREATESYYYMDVWGKGTTVTVSS |
| | Sequence | |
| 106 | BIIB-9-439_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAACCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGAGGCTACAGAAAGCTACTACTACATGGACGTATGGGGCAAGGGTACAACTGTCAC |
| | | CGTCTCCTCA |
| 107 | BIIB-9-446_VH | QVQLVQSGAEVKKPGSSVKVSCKASGGTFSSYAISWVRQAPGQGLEWMGGIIPIFGTASYAQKFQGRVTI |
| | Amino Acid | TADESTSTAYMELSSLRSEDTAVYYCARGLEVGYYGYFDYWGQGTLVTVSS |
| | Sequence | |
| 108 | BIIB-9-446_VH | CAGGTGCAGCTGGTGCAGTCTGGGGCTGAGGTGAAGAAGCCTGGGTCCTCGGTGAAGGTCTCCTGCAAGG |
| | Nucleic Acid | CTTCTGGAGGCACCTTCAGCAGCTATGCTATCAGCTGGGTGCGACAGGCCCCTGGACAAGGGCTTGAGTG |
| | Sequence | GATGGGAGGGATCATCCCTATCTTTGGTACAGCAAGCTACGCACAGAAGTTCCAGGGCAGAGTCACGATT |
| | | ACCGCGGACGAATCCACGAGCACAGCCTACATGGAGCTGAGCAGCCTGAGATCTGAGGACACGGCGGTGT |
| | | ACTACTGCGCCAGAGGGTTGGAAGTGGGATATTATGGATACTTTGATTACTGGGGACAGGGTACATTGGT |
| | | CACCGTCTCCTCA |
| 109 | BIIB-9-568_VH | QLQLQESGPGLVKPSETLSLTCTVSGGSISSSSYYWGWIRQPPGKGLEWIGSIYYSGSTYYNPSLKSRVT |
| | Amino Acid | ISVDTSKNQFSLKLSSVTAADTAVYYCARDLGYAATYFDLWGRGTLVTVSS |
| | Sequence | |
| 110 | BIIB-9-568_VH | CAGCTGCAGCTGCAGGAGTCGGGCCCAGGACTGGTGAAGCCTTCGGAGACCCTGTCCCTCACCTGCACTG |
| | Nucleic Acid | TCTCTGGTGGCTCCATCAGCAGTAGTAGTTACTACTGGGGCTGGATCCGCCAGCCCCCAGGGAAGGGGCT |
| | Sequence | GGAGTGGATTGGGAGTATCTATTATAGTGGGAGCACCTACTACAACCCGTCCCTCAAGAGTCGAGTCACC |
| | | ATATCCGTAGACACGTCCAAGAACCAGTTCTCCCTGAAGCTGAGTTCTGTGACCGCCGCAGACACGGCGG |
| | | TGTACTACTGCGCCAGAGACTTGGGATACGCAGCTACCTACTTCGACCTATGGGGGAGAGGTACCTTGGT |
| | | CACCGTCTCCTCA |
| 111 | BIIB-9-615_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARDSPSSSSYWSLDLWGRGTLVTVSS |
| | Sequence | |
| 112 | BIIB-9-615_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGACTCTCCTAGCAGCAGCTCGTACTGGAGTTTAGACCTATGGGGGAGAGGTACCTT |
| | | GGTCACCGTCTCCTCA |
| 113 | BIIB-9-628_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCAREPIAYGATLDLWGRGTLVTVSS |
| | Sequence | |
| 114 | BIIB-9-628_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGAGCCTATAGCCTACGGTGCTACCTTAGACCTATGGGGGAGAGGTACCTTGGTCAC |
| | | CGTCTCCTCA |
| 115 | BIIB-9-882_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARGPTDSSGYLDMDVWGKGTTVTVSS |
| | Sequence | |
| 116 | BIIB-9-882_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGGACCCACTGACAGCAGCGGATACTTGGACATGGACGTATGGGGCAAGGGTACAAC |
| | | TGTCACCGTCTCCTCA |
| 117 | BIIB-9-884_VH | QLQLQESGPGLVKPSETLSLTCTVSGGSISSSDYYWGWIRQPPGKGLEWIGSIYYSGSTYYNPSLKSRVT |
| | Amino Acid | ISVDTSKNQFSLKLSSVTAADTAVYYCARSPRHKVRGPNWFDPWGQGTLVTVSS |
| | Sequence | |
| 118 | BIIB-9-884_VH | CAGCTGCAGCTGCAGGAGTCGGGCCCAGGACTGGTGAAGCCTTCGGAGACCCTGTCCCTCACCTGCACTG |
| | Amino Acid | TCTCTGGTGGCTCCATCAGCAGTAGTGACTACTACTGGGGCTGGATCCGCCAGCCCCCAGGGAAGGGGCT |
| | Sequence | GGAGTGGATTGGGAGTATCTATTATAGTGGGAGCACCTACTACAACCCGTCCCTCAAGAGTCGAGTCACC |
| | | ATATCCGTAGACACGTCCAAGAACCAGTTCTCCCTGAAGCTGAGTTCTGTGACCGCCGCAGACACGGCGG |
| | | TGTACTACTGCGCCAGATCACCTAGGCACAAAGTGCGTGGCCCCAATTGGTTTGATCCATGGGGACAGGG |
| | | TACATTGGTCACCGTCTCCTCA |
| 119 | BIIB-9-886_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARDPAHSYLDAFDIWGQGTMVTVSS |
| | Sequence | |
| 120 | BIIB-9-886_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGATCCCGCTCACTCCTACCTAGACGCCTTTGATATTTGGGGTCAGGGTACAATGGT |
| | | CACCGTCTCCTCA |
| 121 | BIIB-9-887_VH | QVQLVQSGAEVKKPGASVKVSCKASGYTFTSYYMVWVRQAPGQGLEWMGIINPSGGSTSYAQKFQGRVTM |
| | Amino Acid | TRDTSTSTVYMELSSLRSEDTAVYYCARDAEAHWIPGMDVWGQGTTVTVSS |
| | Sequence | |
| 122 | BIIB-9-887_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGGTCTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGATGCTGAAGCACACTGGATCCCCGGAATGGACGTATGGGGCCAGGGAACAACTGT |
| | | CACCGTCTCCTCA |
| 123 | BIIB-9-888_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARGPTDSSGYLDMDVWGKGTTVTVSS |
| | Sequence | |
| 124 | BIIB-9-888_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGGACCCACTGACAGCAGCGGATACTTGGACATGGACGTATGGGGCAAGGGTACAAC |
| | | TGTCACCGTCTCCTCA |
| 125 | BIIB-9-889_VH | QLQLQESGPGLVKPSETLSLTCTVSGGSISSSSYYWGWIRQPPGKGLEWIGSIYYSGSTYYNPSLKSRVT |
| | Amino Acid | ISVDTSKNQFSLKLSSVTAADTAVYYCARDVGWYTEYFDLWGRGTLVTVSS |
| | Sequence | |
| 126 | BIIB-9-889_VH | CAGCTGCAGCTGCAGGAGTCGGGCCCAGGACTGGTGAAGCCTTCGGAGACCCTGTCCCTCACCTGCACTG |
| | Nucleic Acid | TCTCTGGTGGCTCCATCAGCAGTAGTAGTTACTACTGGGGCTGGATCCGCCAGCCCCCAGGGAAGGGGCT |
| | Sequence | GGAGTGGATTGGGAGTATCTATTATAGTGGGAGCACCTACTACAACCCGTCCCTCAAGAGTCGAGTCACC |
| | | ATATCCGTAGACACGTCCAAGAACCAGTTCTCCCTGAAGCTGAGTTCTGTGACCGCCGCAGACACGGCGG |
| | | TGTACTACTGCGCCAGAGATGTAGGATGGTACACCGAATACTTCGACCTATGGGGGAGAGGTACCTTGGT |
| | | CACCGTCTCCTCA |
| 127 | BIIB-9-433_VH | QLQLQESGPGLVKPSETLSLTCTVSGGSISSSRYYWGWIRQPPGKGLEWIGSIYYSGSTYYNPSLKSRVT |
| | Amino Acid | ISVDTSKNQFSLKLSSVTAADTAVYYCARDAGYSAELFDYWGQGTLVTVSS |
| | Sequence | |
| 128 | BIIB-9-433_VH | CAGCTGCAGCTGCAGGAGTCGGGCCCAGGACTGGTGAAGCCTTCGGAGACCCTGTCCCTCACCTGCACTG |
| | Nucleic Acid | TCTCTGGTGGCTCCATCAGCAGTAGTCGCTACTACTGGGGCTGGATCCGCCAGCCCCCAGGGAAGGGGCT |
| | Sequence | GGAGTGGATTGGGAGTATCTATTATAGTGGGAGCACCTACTACAACCCGTCCCTCAAGAGTCGAGTCACC |
| | | ATATCCGTAGACACGTCCAAGAACCAGTTCTCCCTGAAGCTGAGTTCTGTGACCGCCGCAGACACGGCGG |
| | | TGTACTACTGCGCCAGAGACGCAGGATACAGCGCAGAGTTGTTCGACTACTGGGGACAGGGTACATTGGT |
| | | CACCGTCTCCTCA |
| 129 | BIIB-9-445_VH | QVQLVQSGAEVKKPGASVKVSCKASGYTFTSYYIHWVRQAPGQGLEWMGIINPSGGSTSYAQKFQGRVTM |
| | Amino Acid | TRDTSTSTVYMELSSLRSEDTAVYYCARDVGQDYWFDLWGRGTLVTVSS |
| | Sequence | |
| 130 | BIIB-9-445_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATCCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGATGTAGGACAAGACTACTGGTTCGACCTATGGGGGAGAGGTACCTTGGTCACCGT |
| | | CTCCTCA |
| 131 | BIIB-9-470_VH | EVQLVESGGGLVQPGGSLRLSCAASGFTFSSYWMSWVRQAPGKGLEWVANIKQDGSEKYYVDSVKGRFTI |
| | Amino Acid | SRDNAKNSLYLQMNSLRAEDTAVYYCARDAGIAWALDYWGQGTLVTVS |
| | Sequence | |
| 132 | BIIB-9-470_VH | GAGGTGCAGCTGGTGGAGTCTGGGGGAGGCTTGGTCCAGCCTGGGGGGTCCCTGAGACTCTCCTGTGCAG |
| | Nucleic Acid | CCTCTGGATTCACCTTTAGTAGCTATTGGATGAGCTGGGTCCGCCAGGCTCCAGGGAAGGGGCTGGAGTG |
| | Sequence | GGTGGCCAACATAAAGCAAGATGGAAGTGAGAAATACTATGTGGACTCTGTGAAGGGCCGATTCACCATC |
| | | TCCAGAGACAACGCCAAGAACTCACTGTATCTGCAAATGAACAGCCTGAGAGCCGAGGACACGGCGGTGT |
| | | ACTACTGCGCCAGAGATGCTGGCATAGCCTGGGCCTTAGATTACTGGGGACAGGGTACATTGGTCACCGT |
| | | CTCCTCA |
| 133 | BIIB-9-625_VH | QLQLQESGPGLVKPSETLSLTCTVSGGSISSSSYAWGWIRQPPGKGLEWIGSIYYSGSTYYNPSLKSRVT |
| | Amino Acid | ISVDTSKNQFSLKLSSVTAADTAVYYCARDRGWYTEVLDIWGQGTMVTVSS |
| | Sequence | |
| 134 | BIIB-9-625_VH | CAGCTGCAGCTGCAGGAGTCGGGCCCAGGACTGGTGAAGCCTTCGGAGACCCTGTCCCTCACCTGCACTG |
| | Nucleic Acid | TCTCTGGTGGCTCCATCAGCAGTAGTAGTTACGCATGGGGCTGGATCCGCCAGCCCCCAGGGAAGGGGCT |
| | Sequence | GGAGTGGATTGGGAGTATCTATTATAGTGGGAGCACCTACTACAACCCGTCCCTCAAGAGTCGAGTCACC |
| | | ATATCCGTAGACACGTCCAAGAACCAGTTCTCCCTGAAGCTGAGTTCTGTGACCGCCGCAGACACGGCGG |
| | | TGTACTACTGCGCCAGAGATCGTGGATGGTACACCGAAGTGTTAGACATATGGGGTCAGGGTACAATGGT |
| | | CACCGTCTCCTCA |
| 135 | BIIB-9-1264_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARDGDSSVYAFDYWGQGTLVTVSS |
| | Sequence | |
| 136 | BIIB-9-1264_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGACGGAGACAGCAGCGTGTACGCCTTCGATTATTGGGGACAGGGTACATTGGTCAC |
| | | CGTCTCCTCA |
| 137 | BIIB-9-1265_VH | QLQLQESGPGLVKPSETLSLTCTVSGGSISSSSYYWGWIRQPPGKGLEWIGSIYYSGSTYYNPSLKSRVT |
| | Amino Acid | ISVDTSKNQFSLKLSSVTAADTAVYYCARDGRHYYELFDYWGQGTLVTVSS |
| | Sequence | |
| 138 | BIIB-9-1265_VH | CAGCTGCAGCTGCAGGAGTCGGGCCCAGGACTGGTGAAGCCTTCGGAGACCCTGTCCCTCACCTGCACTG |
| | Nucleic Acid | TCTCTGGTGGCTCCATCAGCAGTAGTAGTTACTACTGGGGCTGGATCCGCCAGCCCCCAGGGAAGGGGCT |
| | Sequence | GGAGTGGATTGGGAGTATCTATTATAGTGGGAGCACCTACTACAACCCGTCCCTCAAGAGTCGAGTCACC |
| | | ATATCCGTAGACACGTCCAAGAACCAGTTCTCCCTGAAGCTGAGTTCTGTGACCGCCGCAGACACGGCGG |
| | | TGTACTACTGCGCCAGAGACGGCAGACACTACTACGAGTTGTTCGACTACTGGGGACAGGGTACATTGGT |
| | | CACCGTCTCCTCA |
| 139 | BIIB-9-1266_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARDHGWAIYGMDVWGQGTTVTVSS |
| | Sequence | |
| 140 | BIIB-9-1266_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCAAGAGACCACGGATGGGCCATCTACGGAATGGACGTATGGGGCCAGGGAACAACTGTCAC |
| | | CGTCTCCTCA |
| 141 | BIIB-9-1267_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARDHGWAIYGMDVWGQGTTVTVSS |
| | Sequence | |
| 142 | BIIB-9-1267_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCAAGAGACCACGGATGGGCCATCTACGGAATGGACGTATGGGGCCAGGGAACAACTGTCAC |
| | | CGTCTCCTCA |
| 143 | BIIB-9-1268_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARDPPSWYVFDIWGQGTMVTVSS |
| | Sequence | |
| 144 | BIIB-9-1268_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGACCCACCTAGTTGGTACGTATTCGACATATGGGGTCAGGGTACAATGGTCACCGT |
| | | CTCCTCA |
| 145 | BIIB-9-1269_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARDRGQYYHFDLWGRGTLVTVSS |
| | Sequence | |
| 146 | BIIB-9-1269_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGATCGTGGACAATACTACCACTTCGACCTATGGGGGAGAGGTACCTTGGTCACCGT |
| | | CTCCTCA |
| 147 | BIIB-9-1270_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARDTGGYAFDIWGQGTLVTVSS |
| | Sequence | |
| 148 | BIIB-9-1270_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGACACGGGAGGATACGCCTTTGATATTTGGGGACAGGGTACATTGGTCACCGTCTC |
| | | CTCA |
| 149 | BIIB-9-1271_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARDTGGYAFDIWGQGTLVTVSS |
| | Sequence | |
| 150 | BIIB-9-1271_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGACACGGGAGGATACGCCTTTGATATTTGGGGACAGGGTACATTGGTCACCGTCTC |
| | | CTCA |
| 151 | BIIB-9-1272_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARDTGGYAFDIWGQGTLVTVSS |
| | Sequence | |
| 152 | BIIB-9-1272_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGACACGGGAGGATACGCCTTTGATATTTGGGGACAGGGTACATTGGTCACCGTCTC |
| | | CTCA |
| 153 | BIIB-9-1273_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARDTGGYAFDIWGQGTLVTVSS |
| | Sequence | |
| 154 | BIIB-9-1273_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGACACGGGAGGATACGCCTTTGATATTTGGGGACAGGGTACATTGGTCACCGTCTC |
| | | CTCA |
| 155 | BIIB-9-1274_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARDTGGYAFDIWGQGTLVTVSS |
| | Sequence | |
| 156 | BIIB-9-1274_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGACACGGGAGGATACGCCTTTGATATTTGGGGACAGGGTACATTGGTCACCGTCTC |
| | | CTCA |
| 157 | BIIB-9-1275_VH | QLQLQESGPGLVKPSETLSLTCTVSGGSISSSSYYWGWIRQPPGKGLEWIGSIYYSGSTYYNPSLKSRVT |
| | Amino Acid | ISVDTSKNQFSLKLSSVTAADTAVYYCARDVGRTYELFDIWGQGTMVTVSS |
| | Sequence | |
| 158 | BIIB-9-1275_VH | CAGCTGCAGCTGCAGGAGTCGGGCCCAGGACTGGTGAAGCCTTCGGAGACCCTGTCCCTCACCTGCACTG |
| | Nucleic Acid | TCTCTGGTGGCTCCATCAGCAGTAGTAGTTACTACTGGGGCTGGATCCGCCAGCCCCCAGGGAAGGGGCT |
| | Sequence | GGAGTGGATTGGGAGTATCTATTATAGTGGGAGCACCTACTACAACCCGTCCCTCAAGAGTCGAGTCACC |
| | | ATATCCGTAGACACGTCCAAGAACCAGTTCTCCCTGAAGCTGAGTTCTGTGACCGCCGCAGACACGGCGG |
| | | TGTACTACTGCGCCAGAGATGTAGGAAGAACCTACGAGCTATTCGACATATGGGGTCAGGGTACAATGGT |
| | | CACCGTCTCCTCA |
| 159 | BIIB-9-1276_VH | QLQLQESGPGLVKPSETLSLTCTVSGGSISSSSYYWGWIRQPPGKGLEWIGSIYYSGSTYYNPSLKSRVT |
| | Amino Acid | ISVDTSKNQFSLKLSSVTAADTAVYYCARDVGRTYELFDIWGQGTMVTVSS |
| | Sequence | |
| 160 | BIIB-9-1276_VH | CAGCTGCAGCTGCAGGAGTCGGGCCCAGGACTGGTGAAGCCTTCGGAGACCCTGTCCCTCACCTGCACTG |
| | Nucleic Acid | TCTCTGGTGGCTCCATCAGCAGTAGTAGTTACTACTGGGGCTGGATCCGCCAGCCCCCAGGGAAGGGGCT |
| | Sequence | GGAGTGGATTGGGAGTATCTATTATAGTGGGAGCACCTACTACAACCCGTCCCTCAAGAGTCGAGTCACC |
| | | ATATCCGTAGACACGTCCAAGAACCAGTTCTCCCTGAAGCTGAGTTCTGTGACCGCCGCAGACACGGCGG |
| | | TGTACTACTGCGCCAGAGATGTAGGAAGAACCTACGAGCTATTCGACATATGGGGTCAGGGTACAATGGT |
| | | CACCGTCTCCTCA |
| 161 | BIIB-9-1277_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARGGTGYYYGSGSRDGYHYYYGMDVWGQGTTVTVSS |
| | Sequence | |
| 162 | BIIB-9-1277_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGGTGGAACTGGGTACTACTACGGAAGCGGAAGCAGAGACGGCTACCACTATTACTA |
| | | CGGCATGGACGTATGGGGCCAGGGAACAACTGTCACCGTCTCCTCA |
| 163 | BIIB-9-1278_VH | QVQLVQSGAEVKKPGASVKVSCKASGYTFTSYYMVWVRQAPGQGLEWMGIINPSGGSTSYAQKFQGRVTM |
| | Amino Acid | TRDTSTSTVYMELSSLRSEDTAVYYCARGPGELGYYLAFDIWGQGTMVTVSS |
| | Sequence | |
| 164 | BIIB-9-1278_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGGTCTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGGACCTGGAGAATTGGGATATTATTTAGCCTTCGATATCTGGGGTCAGGGTACAAT |
| | | GGTCACCGTCTCCTCA |
| 165 | BIIB-9-1279_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARGPTDSSGYLDMDVWGKGTTVTVSS |
| | Sequence | |
| 166 | BIIB-9-1279_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGGACCCACTGACAGCAGCGGATACTTGGACATGGACGTATGGGGCAAGGGTACAAC |
| | | TGTCACCGTCTCCTCA |
| 167 | BIIB-9-1280_VH | QVQLVQSGAEVKKPGASVKVSCKASGYTFTSYYMVWVRQAPGQGLEWMGIINPSGGSTSYAQKFQGRVTM |
| | Amino Acid | TRDTSTSTVYMELSSLRSEDTAVYYCARGPWYSYYYMDVWGKGTTVTVSS |
| | Sequence | |
| 168 | BIIB-9-1280_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGGTCTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGGGCCTTGGTACAGTTATTACTACATGGACGTATGGGGCAAGGGTACAACTGTCAC |
| | | CGTCTCCTCA |
| 169 | BIIB-9-1281_VH | QVQLQQWGAGLLKPSETLSLTCAVYGGSFSGYYWSWIRQPPGKGLEWIGEIDHSGSTNYNPSLKSRVTIS |
| | Amino Acid | VDTSKNQFSLKLSSVTAADTAVYYCARTTRSKYYGMDVWGQGTMVTVSS |
| | Sequence | |
| 170 | BIIB-9-1281_VH | CAAGTACAATTACAACAGTGGGGAGCTGGTTTATTAAAGCCTTCAGAAACTTTAAGTTTGACCTGTGCTG |
| | Nucleic Acid | TTTACGGTGGATCATTTTCTGGTTATTACTGGAGTTGGATTCGTCAACCACCAGGCAAAGGATTGGAGTG |
| | Sequence | GATCGGTGAGATAGACCATTCAGGCTCCACTAACTACAATCCAAGTTTAAAATCCAGGGTTACTATCTCC |
| | | GTAGACACGTCCAAGAACCAGTTCTCCCTGAAGCTGAGTTCTGTGACCGCCGCAGACACGGCGGTGTACT |
| | | ACTGCGCCAGAACTACAAGATCCAAATACTACGGCATGGATGTATGGGGCCAGGGTACAATGGTCACCGT |
| | | CTCCTCA |
| 171 | BIIB-9-1282_VH | QVQLVQSGAEVKKPGASVKVSCKASGYTFTSYYMVWVRQAPGQGLEWMGIINPSGGSTSYAQKFQGRVTM |
| | Amino Acid | TRDTSTSTVYMELSSLRSEDTAVYYCARVPTYRYSYLAFDIWGQGTMVTVSS |
| | Sequence | |
| 172 | BIIB-9-1282_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGGTCTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGTCCCTACATACAGATACAGCTACTTAGCCTTCGATATCTGGGGTCAGGGTACAAT |
| | | GGTCACCGTCTCCTCA |
| 173 | BIIB-9-1283_VH | QVQLVQSGAEVKKPGASVKVSCKASGYTFTSYYMVWVRQAPGQGLEWMGIINPSGGSTSYAQKFQGRVTM |
| | Amino Acid | TRDTSTSTVYMELSSLRSEDTAVYYCARVPTYRYSYLAFDIWGQGTMVTVSS |
| | Sequence | |
| 174 | BIIB-9-1283_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGGTCTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGTCCCTACATACAGATACAGCTACTTAGCCTTCGATATCTGGGGTCAGGGTACAAT |
| | | GGTCACCGTCTCCTCA |
| 175 | BIIB-9-1284_VH | QVQLVQSGAEVKKPGASVKVSCKASGYTFTSYYMVWVRQAPGQGLEWMGIINPSGGSTSYAQKFQGRVTM |
| | Amino Acid | TRDTSTSTVYMELSSLRSEDTAVYYCARVPTYRYSYLAFDIWGQGTMVTVSS |
| | Sequence | |
| 176 | BIIB-9-1284_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCAGGATACACCTTCACCAGCTACTATATGGTCTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGTCCCTACATACAGATACAGCTACTTAGCCTTCGATATCTGGGGTCAGGGTACAAT |
| | | GGTCACCGTCTCCTCA |
| 177 | BIIB-9-1285_VH | QVQLVQSGAEVKKPGASVKVSCKASGYTFTSYYMVWVRQAPGQGLEWMGIINPSGGSTSYAQKFQGRVTM |
| | Amino Acid | TRDTSTSTVYMELSSLRSEDTAVYYCARVPTYRYSYLAFDIWGQGTMVTVSS |
| | Sequence | |
| 178 | BIIB-9-1285_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGGTCTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGTCCCTACATACAGATACAGCTACTTAGCCTTCGATATCTGGGGTCAGGGTACAAT |
| | | GGTCACCGTCTCCTCA |
| 179 | BIIB-9-1286_VH | KPGASVKVSCKASGYTFTSYYMVWVRQAPGQGLEWMGIINPSGGSTSYAQKFQGRVTMTRDTSTSTVYME |
| | Amino Acid | LSSLRSEDTAVYYCARVPTYRYSYLAFDIWGQGTMVTVSS |
| | Sequence | |
| 180 | BIIB-9-1286_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGGTCTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGTCCCTACATACAGATACAGCTACTTAGCCTTCGATATCTGGGGTCAGGGTACAAT |
| | | GGTCACCGTCTCCTCA |
| 181 | BIIB-9-1287_VH | QLQLQESGPGLVKPSETLSLTCTVSGGSISSSDYYWGWIRQPPGKGLEWIGSIYYSGSTYYNPSLKSRVT |
| | Amino Acid | ISVDTSKNQFSLKLSSVTAADTAVYYCARSPRHKVRGPNWFDPWGQGTLVTVSS |
| | Sequence | |
| 182 | BIIB-9-1287_VH | CAGCTGCAGCTGCAGGAGTCGGGCCCAGGACTGGTGAAGCCTTCGGAGACCCTGTCCCTCACCTGCACTG |
| | Nucleic Acid | TCTCTGGTGGCTCCATCAGCAGTAGTGACTACTACTGGGGCTGGATCCGCCAGCCCCCAGGGAAGGGGCT |
| | Sequence | GGAGTGGATTGGGAGTATCTATTATAGTGGGAGCACCTACTACAACCCGTCCCTCAAGAGTCGAGTCACC |
| | | ATATCCGTAGACACGTCCAAGAACCAGTTCTCCCTGAAGCTGAGTTCTGTGACCGCCGCAGACACGGCGG |
| | | TGTACTACTGCGCCAGATCACCTAGGCACAAAGTGCGTGGCCCCAATTGGTTTGATCCATGGGGACAGGG |
| | | TACATTGGTCACCGTCTCCTCA |

Class IV Antibodies - VH Sequences

| | | |
|---|---|---|
| 183 | BIIB-9-397_VH | QVQLQESGPGLVKPSETLSLTCAVSGYSISSGYYWAWIRQPPGKGLEWIGSIYHSGSTYYNPSLKSRVTI |
| | Amino Acid | SVDTSKNQFSLKLSSVTAADTAVYYCARDVWYVGGFDPWGQGTLVTVSS |
| | Sequence | |
| 184 | BIIB-9-397_VH | CAGGTGCAGCTGCAGGAGTCGGGCCCAGGACTGGTGAAGCCTTCGGAGACCCTGTCCCTCACCTGCGCTG |
| | Nucleic Acid | TCTCTGGTTACTCCATCAGCAGTGGTTACTACTGGGCTTGGATCCGGCAGCCCCCAGGGAAGGGGCTGGA |
| | Sequence | GTGGATTGGGAGTATCTATCATAGTGGGAGCACCTACTACAACCCGTCCCTCAAGAGTCGAGTCACCATA |
| | | TCAGTAGACACGTCCAAGAACCAGTTCTCCCTGAAGCTGAGTTCTGTGACCGCCGCAGACACGGCGGTGT |
| | | ACTACTGCGCCAGAGATGTGTGGTACGTCGGCGGTTTCGATCCCTGGGGACAGGGTACATTGGTCACCGT |
| | | CTCCTCA |
| 185 | BIIB-9-578_VH | QVQLQESGPGLVKPSETLSLTCTVSGGSISSSSYYWGWIRQPPGKGLEWIGSISYSGSTYYNPSLKSRVT |
| | Amino Acid | IVDTSKNSQFSLKLSSVTAADTAVYYCARDKYQDYSFDIWGQGTMVTVSS |
| | Sequence | |
| 186 | BIIB-9-578_VH | CAGCTGCAGCTGCAGGAGTCGGGCCCAGGACTGGTGAAGCCTTCGGAGACCCTGTCCCTCACCTGCACTG |
| | Nucleic Acid | TCTCTGGTGGCTCCATCAGCAGTAGTAGTTACTACTGGGGCTGGATCCGCCAGCCCCCAGGGAAGGGGCT |
| | Sequence | GGAGTGGATTGGGAGTATCTCCTATAGTGGGAGCACCTACTACAACCCGTCCCTCAAGAGTCGAGTCACC |
| | | ATATCCGTAGACACGTCCAAGAACCAGTTCTCCCTGAAGCTGAGTTCTGTGACCGCCGCAGACACGGCGG |
| | | TGTACTACTGCGCTAGAGATAAGTACCAAGACTATTCATTCGACATATGGGGTCAGGGTACAATGGTCAC |
| | | CGTCTCCTCA |
| 187 | BIIB-9-631_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARAENRGDYEAWGQGTLVTVSS |
| | Sequence | |
| 188 | BIIB-9-631_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGCCGAGAACAGAGGAGACTACGAGGCATGGGGACAGGGTACATTGGTCACCGTCTC |
| | | CTCA |
| 189 | BIIB-9-612_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARDAGYHWYGMDVWGQGTTVTVSS |
| | Sequence | |
| 190 | BIIB-9-612_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGACGCAGGATACCACTGGTACGGAATGGACGTATGGGGCCAGGGAACAACTGTCAC |
| | | CGTCTCCTCA |

Class I Antibodies - VL Sequences

| | | |
|---|---|---|
| 191 | BIIB-9-605_VL | DIQMTQSPSSVSASVGDRVTITCRASQGISSWLAWYQQKPGKAPKLLIYAASSLQSGVPSRFSGSGSGTD |
| | Amino Acid | FTLTISSLQPEDFATYYCQQADVFPFTFGGGTKVEIK |
| | Sequence | |
| 192 | BIIB-9-605_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGCAGACGTCT |
| | | TCCCTTTCACTTTTGGCGGAGGGACCAAGGTTGAGATCAAA |
| 193 | BIIB-9-475_VL | DIQMTQSPSSLSASVGDRVTITCQASQDITNYLNWYQQKPGKAPKLLIYDASNLETGVPSRFSGSGSGTD |
| | Amino Acid | FTFTISSLQPEDIATYYCQQSSNFPLTFGGGTKVEIK |
| | Sequence | |
| 194 | BIIB-9-475_VL | GACATCCAGATGACCCAGTCTCCATCCTCCCTGTCTGCATCTGTAGGAGACAGAGTCACCATCACTTGCC |
| | Nucleic Acid | AGGCGAGTCAGGACATTACCAACTATTTAAATTGGTATCAGCAGAAACCAGGGAAAGCCCCTAAGCTCCT |
| | Sequence | GATCTACGATGCATCCAATTTGGAAACAGGGGTCCCATCAAGGTTCAGTGGAAGTGGATCTGGGACAGAT |
| | | TTTACTTTCACCATCAGCAGCCTGCAGCCTGAAGATATTGCAACATATTACTGTCAGCAGTCCTCCAATT |
| | | TCCCTCTCACTTTTGGCGGAGGGACCAAGGTTGAGATCAAA |
| 195 | BIIB-9-477_VL | EIVMTQSPATLSVSPGERATLSCRASQSVSSSLAWYQQKPGQAPRLLIFGASTRATGIPARFSGSGSGTE |
| | Amino Acid | FTLTISSLQSEDFAVYYCQQDVNWPITFGGGTKVEIK |
| | Sequence | |
| 196 | BIIB-9-477_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAGCTTAGCCTGGTACCAGCAGAAACCTGGCCAGGCTCCCAGGCTCCT |
| | Sequence | CATCTTTGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGGACGTCAATT |
| | | GGCCTATCACTTTTGGCGGAGGGACCAAGGTTGAGATCAAA |
| 197 | BIIB-9-479_VL | DIQMTQSPSTLSASVGDRVTITCRASQSISSWLAWYQQKPGKAPKLLIYKASSLESGVPSRFSGSGSGTE |
| | Amino Acid | FTLTISSLQPDDFATYYCQQYRILSPTFGGGTKVEIK |
| | Sequence | |
| 198 | BIIB-9-479_VL | GACATCCAGATGACCCAGTCTCCTTCCACCCTGTCTGCATCTGTAGGAGACAGAGTCACCATCACTTGCC |
| | Nucleic Acid | GGGCCAGTCAGAGTATTAGTAGCTGGTTGGCCTGGTATCAGCAGAAACCAGGGAAAGCCCCTAAGCTCCT |
| | Sequence | GATCTATAAAGCCTCCAGTTTGGAAAGTGGGGTCCCATCAAGGTTCAGCGGCAGTGGATCTGGGACAGAA |
| | | TTCACTCTCACCATCAGCAGCCTGCAGCCTGATGATTTTGCAACTTATTACTGCCAGCAGTACCGCATCC |
| | | TCTCTCCTACTTTTGGCGGAGGGACCAAGGTTGAGATCAAA |
| 199 | BIIB-9-480_VL | DIVMTQSPLSLPVTPGEPASISCRSSQSLLYSNGYNYLDWYLQKPGQSPQLLIYLGSNRASGVPDRFSGS |
| | Amino Acid | GSGTDFTLKISRVEAEDVGVYYCMQARQPPWTFGGGTKVE1K |
| | Sequence | |
| 200 | BIIB-9-480_VL | GATATTGTGATGACTCAGTCTCCACTCTCCCTGCCCGTCACCCCTGGAGAGCCGGCCTCCATCTCCTGCA |
| | Nucleic Acid | GGTCTAGTCAGAGCCTCCTGTATAGTAATGGATACAACTATTTGGATTGGTACCTGCAGAAGCCAGGGCA |
| | Sequence | GTCTCCACAGCTCCTGATCTATTTGGGTTCTAATCGGGCCTCCGGGGTCCCTGACAGGTTCAGTGGCAGT |
| | | GGATCAGGCACAGATTTTACACTGAAAATCAGCAGAGTGGAGGCTGAGGATGTTGGGGTTTATTACTGCA |
| | | TGCAGGCACGACAGCCCCCTTGGACTTTTGGCGGAGGGACCAAGGTTGAGATCAAA |
| 201 | BIIB-9-558_VL | DIQMTQSPSTLSASVGDRVTITCRASQSIGSWLAWYQQKPGKAPKLLIYKASSLESGVPSRFSGSGSGTE |
| | Amino Acid | FTLTISSLQPDDFATYYCQQAGSYSFTFGGGTKVEIK |
| | Sequence | |
| 202 | BIIB-9-558_VL | GACATCCAGATGACCCAGTCTCCTTCCACCCTGTCTGCATCTGTAGGAGACAGAGTCACCATCACTTGCC |
| | Nucleic Acid | GGGCCAGTCAGAGTATTGGTAGCTGGTTGGCCTGGTATCAGCAGAAACCAGGGAAAGCCCCTAAGCTCCT |
| | Sequence | GATCTATAAAGCCTCCAGTTTGGAAAGTGGGGTCCCATCAAGGTTCAGCGGCAGTGGATCTGGGACAGAA |
| | | TTCACTCTCACCATCAGCAGCCTGCAGCCTGATGATTTTGCAACTTATTACTGCCAGCAGGCCGGAAGCT |
| | | ACTCTTTCACTTTTGGCGGAGGGACCAAGGTTGAGATCAAA |
| 203 | BIIB-9-414_VL | DIQMTQSPSSVSASVGDRVTITCRASQGISSWLAWYQQKPGKAPKLLIYAASSLQSGVPSRFSGSGSGTD |
| | Amino Acid | FTLTISSLQPEDFATYYCQQGDVFPFTFGGGTKVEIK |
| | Sequence | |
| 204 | BIIB-9-414_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGGAGACGTCT |
| | | TCCCTTTCACTTTTGGCGGAGGGACCAAGGTTGAGATCAAA |
| 205 | BIIB-9-415_VL | DIQMTQSPSSVSASVGDRVTITCRASQGIDSWLAWYQQKPGKAPKLLIYAASSLQSGVPSRFSGSGSGTD |
| | Amino Acid | FTLTISSLQPEDFATYYCQQGDAFPFTFGGGTKVEIK |
| | Sequence | |
| 206 | BIIB-9-415_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTGACAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGGAGACGCCT |
| | | TCCCTTTCACTTTTGGCGGAGGGACCAAGGTTGAGATCAAA |
| 207 | BIIB-9-425_VL | EIVLTQSPGTLSLSPGERATLSCRASQSVSSDYLAWYQQKPGQAPRLLIYGASSRATGIPDRFSGSGSGT |
| | Amino Acid | DFTLTISRLEPEDFAVYYCQQYDSHPYTFGGGTKVEIK |
| | Sequence | |
| 208 | BIIB-9-425_VL | GAAATTGTGTTGACGCAGTCTCCAGGCACCCTGTCTTTGTCTCCAGGGGAAAGAGCCACCCTCTCCTGCA |
| | Nucleic Acid | GGGCCAGTCAGAGTGTTAGCAGCGACTACTTAGCCTGGTACCAGCAGAAACCTGGCCAGGCTCCCAGGCT |
| | Sequence | CCTCATCTATGGTGCATCCAGCAGGGCCACTGGCATCCCAGACAGGTTCAGTGGCAGTGGGTCTGGGACA |
| | | GACTTCACTCTCACCATCAGCAGACTGGAGCCTGAAGATTTTGCAGTGTATTACTGTCAGCAGTACGACA |
| | | GTCACCCTTACACTTTTGGCGGAGGGACCAAGGTTGAGATCAAA |
| 209 | BIIB-9-440_VL | DIVMTQSPDSLAVSLGERATINCKSSQSVLYSSNNKNYLAWYQQKPGQPPKLLIYWASTRESGVPDRFSG |
| | Amino Acid | SGSGTDFTLTISSLQAEDVAVYYCQQYALDPPTFGGGTKVEIK |
| | Sequence | |
| 210 | BIIB-9-440_VL | GACATCGTGATGACCCAGTCTCCAGACTCCCTGGCTGTGTCTCTGGGCGAGAGGGCCACCATCAACTGCA |
| | Nucleic Acid | AGTCCAGCCAGAGTGTTTTATACAGCTCCAACAATAAGAACTACTTAGCTTGGTACCAGCAGAAACCAGG |
| | Sequence | ACAGCCTCCTAAGCTGCTCATTTACTGGGCATCTACCCGGGAATCCGGGGTCCCTGACCGATTCAGTGGC |
| | | AGCGGGTCTGGGACAGATTTCACTCTCACCATCAGCAGCCTGCAGGCTGAAGATGTGGCAGTTTATTACT |
| | | GTCAGCAGTACGCCCTCGACCCTCCTACTTTTGGCGGAGGGACCAAGGTTGAGATCAAA |
| 211 | BIIB-9-452_VL | EIVMTQSPATLSVSPGERATLSCRASQSVSSNLAWYQQKPGQAPRLLIYGASTRATGIPARFSGSGSGTE |
| | Amino Acid | FTLTISSLQSEDFAVYYCQQANNFPFTFGGGTKVEIK |
| | Sequence | |
| 212 | BIIB-9-452_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGGCCAATAATT |
| | | TCCCTTTCACTTTTGGCGGAGGGACCAAGGTTGAGATCAAA |
| 213 | BIIB-9-460_VL | EIVMTQSPATLSVSPGERATLSCRASQSVSSNLAWYQQKPGQAPRLLIYGASTRATGIPARFSGSGSGTE |
| | Amino Acid | FTLTISSLQSEDFAVYYCQQHDNFPFTFGGGTKVEIK |
| | Sequence | |
| 214 | BIIB-9-460_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGCACGACAATT |
| | | TCCCTTTCACTTTTGGCGGAGGGACCAAGGTTGAGATCAAA |
| 215 | BIIB-9-461_VL | EIVMTQSPATLSVSPGERATLSCRASQSVSSSLAWYQQKPGQAPRLLIYGASTRATGIPARFSGSGSGTE |
| | Amino Acid | FTLTISSLQSEDFAVYYCQQHHHWPPTFGGGTKVEIK |
| | Sequence | |
| 216 | BIIB-9-461_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAGCTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGCACCACCACT |
| | | GGCCTCCTACTTTTGGCGGAGGGACCAAGGTTGAGATCAAA |
| 217 | BIIB-9-465_VL | DIQMTQSPSTLSASVGDRVTITCRASQSINSWLAWYQQKPGKAPKLLISDASSLESGVPSRFSGSGSGTE |
| | Amino Acid | FTLTISSLQPDDFATYYCQQYEIFPFTFGGGTKVEIK |
| | Sequence | |
| 218 | BIIB-9-465_VL | GACATCCAGATGACCCAGTCTCCTTCCACCCTGTCTGCATCTGTAGGAGACAGAGTCACCATCACTTGCC |
| | Nucleic Acid | GGGCCAGTCAGAGTATTAATAGCTGGTTGGCCTGGTATCAGCAGAAACCAGGGAAAGCCCCTAAGCTCCT |
| | Sequence | GATCTCCGATGCCTCCAGTTTGGAAAGTGGGGTCCCATCAAGGTTCAGCGGCAGTGGATCTGGGACAGAA |
| | | TTCACTCTCACCATCAGCAGCCTGCAGCCTGATGATTTTGCAACTTATTACTGCCAGCAGTACGAAATCT |
| | | TCCCTTTCACTTTTGGCGGAGGGACCAAGGTTGAGATCAAA |
| 219 | BIB-4-564_VL | DIQMTQSPSSLSASVGDRVTITCRASQSISSYLNWYQQKPGKAPKLLIYAASSLQSGVPSRFSGSGSGTD |
| | Amino Acid | FTLTISSLQPEDFATYYCQQSVAVPPTFGGGTKVEIK |
| | Sequence | |
| 220 | BIIB-9-564_VL | GACATCCAGATGACCCAGTCTCCATCCTCCCTGTCTGCATCTGTAGGAGACAGAGTCACCATCACTTGCC |
| | Nucleic Acid | GGGCAAGTCAGAGCATTAGCAGCTATTTAAATTGGTATCAGCAGAAACCAGGGAAAGCCCCTAAGCTCCT |
| | Sequence | GATCTATGCTGCATCCAGTTTGCAAAGTGGGGTCCCATCAAGGTTCAGTGGCAGTGGATCTGGGACAGAT |
| | | TTCACTCTCACCATCAGCAGTCTGCAACCTGAAGATTTTGCAACTTACTACTGTCAGCAAAGCGTCGCCG |
| | | TCCCTCCTACTTTTGGCGGAGGGACCAAGGTTGAGATCAAA |
| 221 | BIIB-9-484_VL | DIQMTQSPSSLSASVGDRVTITCQASQDIANYLNWYQQKPGKAPKLLIYDASNLETGVPSRFSGSGSGTD |
| | Amino Acid | FTFTISSLQPEDIATYYCQQYANFPYTFGGGTKVEIK |
| | Sequence | |
| 222 | BIIB-9-484_VL | GACATCCAGATGACCCAGTCTCCATCCTCCCTGTCTGCATCTGTAGGAGACAGAGTCACCATCACTTGCC |
| | Nucleic Acid | AGGCGAGTCAGGACATTGCCAACTATTTAAATTGGTATCAGCAGAAACCAGGGAAAGCCCCTAAGCTCCT |
| | Sequence | GATCTACGATGCATCCAATTTGGAAACAGGGGTCCCATCAAGGTTCAGTGGAAGTGGATCTGGGACAGAT |
| | | TTTACTTTCACCATCAGCAGCCTGCAGCCTGAAGATATTGCAACATATTACTGTCAGCAGTACGCCAACT |
| | | TCCCTTACACTTTTGGCGGAGGGACCAAGGTTGAGATCAAA |
| 223 | BIIB-9-469_VL | EIVMTQSPATLSVSPGERATLSCRASQSVSSNLAWYQQKPGQAPRLLIFGASTRATGIPARFSGSGSGTE |
| | Amino Acid | FTLTISSLQSEDFAVYYCQQSNNFPFTFGGGTKVEIK |
| | Sequence | |
| 224 | BIIB-9-469_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTTTGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGTCCAATAATT |
| | | TCCCTTTCACTTTTGGCGGAGGGACCAAGGTTGAGATCAAA |
| 225 | BIIB-9-566_VL | EIVMTQSPATLSVSPGERATLSCRASQSVSSSLAWYQQKPGQAPRLLIYGASTRATGIPARFSGSGSGTE |
| | Amino Acid | FTLTISSLQSEDFAVYYCQQHDNWPPTFGGGTKVEIK |
| | Sequence | |
| 226 | BIIB-9-566_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAGCTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGCACGACAATT |
| | | GGCCTCCTACTTTTGGCGGAGGGACCAAGGTTGAGATCAAA |
| 227 | BIIB-9-567_VL | DIQMTQSPSTLSASVGDRVTITCRASQSISSWLAWYQQKPGKAPKLLIYDASSLESGVPSRFSGSGSGTE |
| | Amino Acid | FTLTISSLQPDDFATYYCQQYRIYSPTFGGGTKVEIK |
| | Sequence | |
| 228 | BIIB-9-567_VL | GACATCCAGATGACCCAGTCTCCTTCCACCCTGTCTGCATCTGTAGGAGACAGAGTCACCATCACTTGCC |
| | Nucleic Acid | GGGCCAGTCAGAGTATTAGTAGCTGGTTGGCCTGGTATCAGCAGAAACCAGGGAAAGCCCCTAAGCTCCT |
| | Sequence | GATCTATGATGCCTCCAGTTTGGAAAGTGGGGTCCCATCAAGGTTCAGCGGCAGTGGATCTGGGACAGAA |
| | | TTCACTCTCACCATCAGCAGCCTGCAGCCTGATGATTTTGCAACTTATTACTGCCAGCAGTACAGAATCT |
| | | ACTCTCCTACTTTTGGCGGAGGGACCAAGGTTGAGATCAAA |
| 229 | BIIB-9-569_VL | DIQMTQSPSSLSASVGDRVTITCQASQDISNYLNWYQQKPGKAPKLLIYDASNLATGVPSRFSGSGSGTD |
| | Amino Acid | FTFTISSLQPEDIATYYCQQADDFPFTFGGGTKVEIK |
| | Sequence | |
| 230 | BIIB-9-569_VL | GACATCCAGATGACCCAGTCTCCATCCTCCCTGTCTGCATCTGTAGGAGACAGAGTCACCATCACTTGCC |
| | Nucleic Acid | AGGCGAGTCAGGACATTAGCAACTATTTAAATTGGTATCAGCAGAAACCAGGGAAAGCCCCTAAGCTCCT |
| | Sequence | GATCTACGATGCATCCAATTTGGCAACAGGGGTCCCATCAAGGTTCAGTGGAAGTGGATCTGGGACAGAT |
| | | TTTACTTTCACCATCAGCAGCCTGCAGCCTGAAGATATTGCAACATATTACTGTCAGCAGGCCGATGACT |
| | | TCCCTTTCACTTTTGGCGGAGGGACCAAGGTTGAGATCAAA |
| 231 | BIIB-9-588_VL | EIVMTQSPATLSVSPGERATLSCRASQSVSSNLAWYQQKPGQAPRLLIYGASTRATGIPARFSGSGSGTE |
| | Amino Acid | FTLTISSLQSEDFAVYYCQQAYNWPFTFGGGTKVEIK |
| | Sequence | |
| 232 | BIIB-9-588_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGGCCTACAATT |
| | | GGCCTTTCACTTTTGGCGGAGGGACCAAGGTTGAGATCAAA |
| 233 | BIIB-9-611_VL | EIVMTQSPATLSVSPGERATLSCRASQSVSSNLAWYQQKPGQAPRLLIYGASTRATGIPARFSGSGSGTE |
| | Amino Acid | FTLTISSLQSEDFAVYYCQQDNIHPYTFGGGTKVEIK |
| | Sequence | |
| 234 | BIIB-9-611_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGGACAATATCC |
| | | ACCCTTACACTTTTGGCGGAGGGACCAAGGTTGAGATCAAA |
| 235 | BIIB-9-619_VL | EIVLTQSPATLSLSPGERATLSCRASQSVSSYLAWYQQKPGQAPRLLIYDASNRATGIPARFSGSGSGTD |
| | Amino Acid | FTLTISSLEPEDFAVYYCQQRDNWPFTFGGGTKVEIK |
| | Sequence | |
| 236 | BIIB-9-619_VL | GAAATTGTGTTGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGAGAGACAACT |
| | | GGCCTTTCACTTTTGGCGGAGGGACCAAGGTTGAGATCAAA |
| 237 | BIIB-9-626_VL | DIQMTQSPSTLSASVGDRVTITCRASQSISSWLAWYQQKPGKAPKLLIYKASSLESGVPSRFSGSGSGTE |
| | Amino Acid | FTLTISSLQPDDFATYYCQQDGSYPPLTFGGGTKVEIK |
| | Sequence | |
| 238 | BIIB-9-626_VL | GACATCCAGATGACCCAGTCTCCTTCCACCCTGTCTGCATCTGTAGGAGACAGAGTCACCATCACTTGCC |
| | Nucleic Acid | GGGCCAGTCAGAGTATTAGTAGCTGGTTGGCCTGGTATCAGCAGAAACCAGGGAAAGCCCCTAAGCTCCT |
| | Sequence | GATCTATAAAGCCTCCAGTTTGGAAAGTGGGGTCCCATCAAGGTTCAGCGGCAGTGGATCTGGGACAGAA |
| | | TTCACTCTCACCATCAGCAGCCTGCAGCCTGATGATTTTGCAACTTATTACTGCCAGCAGGACGGAAGTT |
| | | ACCCTCCTCTCACTTTTGGCGGAGGGACCAAGGTTGAGATCAAA |
| 239 | BIIB-9-883_VL | DIQMTQSPSSLSASVGDRVTITCQASQDITNYLNWYQQKPGKAPKLLIYDASNLETGVPSRFSGSGSGTD |
| | Amino Acid | FTFTISSLQPEDIATYYCQQADHFPFTFGGGTKVEIK |
| | Sequence | |
| 240 | BIIB-9-883_VL | GACATCCAGATGACCCAGTCTCCATCCTCCCTGTCTGCATCTGTAGGAGACAGAGTCACCATCACTTGCC |
| | Nucleic Acid | AGGCGAGTCAGGACATTACCAACTATTTAAATTGGTATCAGCAGAAACCAGGGAAAGCCCCTAAGCTCCT |
| | Sequence | GATCTACGATGCATCCAATTTGGAAACAGGGGTCCCATCAAGGTTCAGTGGAAGTGGATCTGGGACAGAT |
| | | TTTACTTTCACCATCAGCAGCCTGCAGCCTGAAGATATTGCAACATATTACTGTCAGCAGGCCGATCACT |
| | | TCCCTTTCACTTTTGGCGGAGGGACCAAGGTTGAGATCAAA |
| 241 | BIIB-9-419_VL | EIVMTQSPATLSLSPGERATLSCRASQSVSSYLAWYQQKPGQAPRLLIYDSSNRATGIPARFSGSGSGTD |
| | Amino Acid | FTLTISSLEPEDFAVYYCQQVSTHPYTFGGGTKVEIK |
| | Sequence | |
| 242 | BIIB-9-419_VL | GAAATTGTGATGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATTCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGGTCAGTACCC |
| | | ACCCTTACACTTTTGGCGGAGGGACCAAGGTTGAGATCAAA |
| 243 | BIIB-9-451_VL | DIVMTQSPDSLAVSLGERATINCKSSQSVLYSSNNKNYLAWYQQKPGQPPKLLIYWASTRESGVPDRFSG |
| | Amino Acid | SGSGTDFTLTISSLQAEDVAVYYCQQYYFPPWTFGGGTKVEIK |
| | Sequence | |
| 244 | BIIB-9-451_VL | GACATCGTGATGACCCAGTCTCCAGACTCCCTGGCTGTGTCTCTGGGCGAGAGGGCCACCATCAACTGCA |
| | Nucleic Acid | AGTCCAGCCAGAGTGTTTTATACAGCTCCAACAATAAGAACTACTTAGCTTGGTACCAGCAGAAACCAGG |
| | Sequence | ACAGCCTCCTAAGCTGCTCATTTACTGGGCATCTACCCGGGAATCCGGGGTCCCTGACCGATTCAGTGGC |
| | | AGCGGGTCTGGGACAGATTTCACTCTCACCATCAGCAGCCTGCAGGCTGAAGATGTGGCAGTTTATTACT |
| | | GTCAGCAGTACTACTTCCCCCCTTGGACTTTTGGCGGAGGGACCAAGGTTGAGATCAAA |
| 245 | BIIB-9-473_VL | EIVLTQSPATLSLSPGERATLSCRASQSVSSYLAWYQQKPGQAPRLLIYDASNRATGIPARFSGSGSGTD |
| | Amino Acid | FTLTISSLEPEDFAVYYCQQRSFLPYTFGGGTKVEIK |
| | Sequence | |
| 246 | BIIB-9-473_VL | GAAATTGTGTTGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGAGAAGTTTCC |
| | | TCCCTTACACTTTTGGCGGAGGGACCAAGGTTGAGATCAAA |
| 247 | BIIB-9-565_VL | EIVLTQSPATLSLSPGERATLSCRASQSVSSYLAWYQQKPGQAPRLLIYDASNRATGIPARFSGSGSGTD |
| | Amino Acid | FTLTISSLEPEDFAVYYCQQDSNLPFTFGGGTKVEIK |
| | Sequence | |
| 248 | BIIB-9-565_VL | GAAATTGTGTTGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGGACAGTAATC |
| | | TCCCTTTCACTTTTGGCGGAGGGACCAAGGTTGAGATCAAA |
| 249 | BIIB-9-573_VL | EIVLTQSPGTLSLSPGERATLSCRASQSVSSSFLAWYQQKPGQAPRLLIYGASSRATGIPDRFSGSGSGT |
| | Amino Acid | DFTLTISRLEPEDFAVYYCQQSHSPPYTFGGGTKVEIK |
| | Sequence | |
| 250 | BIIB-9-573_VL | GAAATTGTGTTGACGCAGTCTCCAGGCACCCTGTCTTTGTCTCCAGGGGAAAGAGCCACCCTCTCCTGCA |
| | Nucleic Acid | GGGCCAGTCAGAGTGTTAGCAGCAGCTTCTTAGCCTGGTACCAGCAGAAACCTGGCCAGGCTCCCAGGCT |
| | Sequence | CCTCATCTATGGTGCATCCAGCAGGGCCACTGGCATCCCAGACAGGTTCAGTGGCAGTGGGTCTGGGACA |
| | | GACTTCACTCTCACCATCAGCAGACTGGAGCCTGAAGATTTTGCAGTGTATTACTGTCAGCAGTCCCACA |
| | | GTCCCCCTTACACTTTTGGCGGAGGGACCAAGGTTGAGATCAAA |
| 251 | BIIB-9-579_VL | DIQMTQSPSSLSASVGDRVTITCRASQSISSYLNWYQQKPGKAPKLLIYGASSLQSGVPSRFSGSGSGTD |
| | Amino Acid | FTLTISSLQPEDFATYYCQQAFSFPFTFGGGTKVEIK |
| | Sequence | |
| 252 | BIIB-9-579_VL | GACATCCAGATGACCCAGTCTCCATCCTCCCTGTCTGCATCTGTAGGAGACAGAGTCACCATCACTTGCC |
| | Nucleic Acid | GGGCAAGTCAGAGCATTAGCAGCTATTTAAATTGGTATCAGCAGAAACCAGGGAAAGCCCCTAAGCTCCT |
| | Sequence | GATCTATGGTGCATCCAGTTTGCAAAGTGGGGTCCCATCAAGGTTCAGTGGCAGTGGATCTGGGACAGAT |
| | | TTCACTCTCACCATCAGCAGTCTGCAACCTGAAGATTTTGCAACTTACTACTGTCAGCAAGCATTCAGTT |
| | | TCCCTTTCACTTTTGGCGGAGGGACCAAGGTTGAGATCAAA |
| 253 | BIIB-9-581_VL | DIQMTQSPSSLSASVGDRVTITCRASQSISSYLNWYQQKPGKAPKLLIYAASSLQSGVPSRFSGSGSGTD |
| | Amino Acid | FTLTISSLQPEDFATYYCQQSDLFPFTFGGGTKVEIK |
| | Sequence | |
| 254 | BIIB-9-581_VL | GACATCCAGATGACCCAGTCTCCATCCTCCCTGTCTGCATCTGTAGGAGACAGAGTCACCATCACTTGCC |
| | Nucleic Acid | GGGCAAGTCAGAGCATTAGCAGCTATTTAAATTGGTATCAGCAGAAACCAGGGAAAGCCCCTAAGCTCCT |
| | Sequence | GATCTATGCTGCATCCAGTTTGCAAAGTGGGGTCCCATCAAGGTTCAGTGGCAGTGGATCTGGGACAGAT |
| | | TTCACTCTCACCATCAGCAGTCTGCAACCTGAAGATTTTGCAACTTACTACTGTCAGCAAAGCGACCTCT |
| | | TCCCTTTCACTTTTGGCGGAGGGACCAAGGTTGAGATCAAA |
| 255 | BIIB-9-582_VL | DIQMTQSPSSLSASVGDRVTITCQASQDISNYLNWYQQKPGKAPKLLIYDASNLETGVPSRFSGSGSGTD |
| | Amino Acid | FTFTISSLQPEDIATYYCQQADILPPTFGGGTKVEIK |
| | Sequence | |
| 256 | BIIB-9-582_VL | GACATCCAGATGACCCAGTCTCCATCCTCCCTGTCTGCATCTGTAGGAGACAGAGTCACCATCACTTGCC |
| | Nucleic Acid | AGGCGAGTCAGGACATTAGCAACTATTTAAATTGGTATCAGCAGAAACCAGGGAAAGCCCCTAAGCTCCT |
| | Sequence | GATCTACGATGCATCCAATTTGGAAACAGGGGTCCCATCAAGGTTCAGTGGAAGTGGATCTGGGACAGAT |
| | | TTTACTTTCACCATCAGCAGCCTGCAGCCTGAAGATATTGCAACATATTACTGTCAGCAGGCCGATATCC |
| | | TCCCTCCTACTTTTGGCGGAGGGACCAAGGTTGAGATCAAA |
| 257 | BIIB-9-585_VL | DIQMTQSPSSVSASVGDRVTITCRASQGISSWLAWYQQKPGKAPKLLIYAASSLQSGVPSRFSGSGSGTD |
| | Amino Acid | FTLTISSLQPEDFATYYCQQANSYPITFGGGTKVEIK |
| | Sequence | |
| 258 | BIIB-9-585_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGCAAATAGTT |
| | | ACCCTATCACTTTTGGCGGAGGGACCAAGGTTGAGATCAAA |
| 259 | BIIB-9-587_VL | DIQMTQSPSSVSASVGDRVTITCRASQGISSWLAWYQQKPGKAPKLLIYAASSLQSGVPSRFSGSGSGTD |
| | Amino Acid | FTLTISSLQPEDFATYYCQQADSFPFTFGGGTKVEIK |
| | Sequence | |
| 260 | BIIB-9-587_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGCAGACAGTT |
| | | TCCCTTTCACTTTTGGCGGAGGGACCAAGGTTGAGATCAAA |
| 261 | BIIB-9-590_VL | EIVLTQSPATLSLSPGERATLSCRASQSVSSYLAWYQQKPGQAPRLLIYDASNRATGIPARFSGSGSGTD |
| | Amino Acid | FTLTISSLEPEDFAVYYCQQVDNFPLTFGGGTKVEIK |
| | Sequence | |
| 262 | BIIB-9-590_VL | GAAATTGTGTTGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGGTCGACAATT |
| | | TCCCTCTCACTTTTGGCGGAGGGACCAAGGTTGAGATCAAA |
| 263 | BIIB-9-592_VL | DIQMTQSPSSLSASVGDRVTITCRASQSISSYLNWYQQKPGKAPKLLIYAASSLQSGVPSRFSGSGSGTD |
| | Amino Acid | FTLTISSLQPEDFATYYCQQPYDTPITFGGGTKVEIK |
| | Sequence | |
| 264 | BIIB-9-592_VL | GACATCCAGATGACCCAGTCTCCATCCTCCCTGTCTGCATCTGTAGGAGACAGAGTCACCATCACTTGCC |
| | Nucleic Acid | GGGCAAGTCAGAGCATTAGCAGCTATTTAAATTGGTATCAGCAGAAACCAGGGAAAGCCCCTAAGCTCCT |
| | Sequence | GATCTATGCTGCATCCAGTTTGCAAAGTGGGGTCCCATCAAGGTTCAGTGGCAGTGGATCTGGGACAGAT |
| | | TTCACTCTCACCATCAGCAGTCTGCAACCTGAAGATTTTGCAACTTACTACTGTCAGCAACCATACGACA |
| | | CTCCTATCACTTTTGGCGGAGGGACCAAGGTTGAGATCAAA |
| 265 | BIIB-9-606_VL | DIQMTQSPSSLSASVGDRVTITCQASQDISNYLNWYQQKPGKAPKLLIYDASNLETGVPSRFSGSGSGTD |
| | Amino Acid | FTFTISSLQPEDIATYYCQQSDLFPFTFGGGTKVEIK |
| | Sequence | |
| 266 | BIIB-9-606_VL | GACATCCAGATGACCCAGTCTCCATCCTCCCTGTCTGCATCTGTAGGAGACAGAGTCACCATCACTTGCC |
| | Nucleic Acid | AGGCGAGTCAGGACATTAGCAACTATTTAAATTGGTATCAGCAGAAACCAGGGAAAGCCCCTAAGCTCCT |
| | Sequence | GATCTACGATGCATCCAATTTGGAAACAGGGGTCCCATCAAGGTTCAGTGGAAGTGGATCTGGGACAGAT |
| | | TTTACTTTCACCATCAGCAGCCTGCAGCCTGAAGATATTGCAACATATTACTGTCAGCAGTCCGATCTCT |
| | | TCCCTTTCACTTTTGGCGGAGGGACCAAGGTTGAGATCAAA |
| 267 | BIIB-9-608_VL | DIVMTQSPDSLAVSLGERATINCKSSQSVLYSSNNKNYLAWYQQKPGQPPKLLIYWASTRESGVPDRFSG |
| | Amino Acid | SGSGTDFTLTISSLQAEDVAVYYCQQFLYTPTFGGGTKVEIK |
| | Sequence | |
| 268 | BIIB-9-608_VL | GACATCGTGATGACCCAGTCTCCAGACTCCCTGGCTGTGTCTCTGGGCGAGAGGGCCACCATCAACTGCA |
| | Nucleic Acid | AGTCCAGCCAGAGTGTTTTATACAGCTCCAACAATAAGAACTACTTAGCTTGGTACCAGCAGAAACCAGG |
| | Sequence | ACAGCCTCCTAAGCTGCTCATTTACTGGGCATCTACCCGGGAATCCGGGGTCCCTGACCGATTCAGTGGC |
| | | AGCGGGTCTGGGACAGATTTCACTCTCACCATCAGCAGCCTGCAGGCTGAAGATGTGGCAGTTTATTACT |
| | | GTCAGCAGTTCCTCTACACTCCTACTTTTGGCGGAGGGACCAAGGTTGAGATCAAA |
| 269 | BIIB-9-616_VL | EIVMTQSPATLSVSPGERATLSCRASQSVSSSLAWYQQKPGQAPRLLIYGASTRATGIPARFSGSGSGTE |
| | Amino Acid | FTLTISSLQSEDFAVYYCQQADNFPFTFGGGTKVEIK |
| | Sequence | |
| 270 | BIIB-9-616_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAGCTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGGCCGACAATT |
| | | TCCCTTTCACTTTTGGCGGAGGGACCAAGGTTGAGATCAAA |
| 271 | BIIB-9-621_VL | DIVMTQSPDSLAVSLGERATINCKSSQSVLYSSNNKNYLAWYQQKPGQPPKLLIYWASTRESGVPDRFSG |
| | Amino Acid | SGSGTDFTLTISSLQAEDVAVYYCQQFYLPPWTFGGGTKVEIK |
| | Sequence | |
| 272 | BIIB-9-621_VL | GACATCGTGATGACCCAGTCTCCAGACTCCCTGGCTGTGTCTCTGGGCGAGAGGGCCACCATCAACTGCA |
| | Nucleic Acid | AGTCCAGCCAGAGTGTTTTATACAGCTCCAACAATAAGAACTACTTAGCTTGGTACCAGCAGAAACCAGG |
| | Sequence | TACAGCCTCCTAAGCTGCTCATTTACGGGCATCTACCCGGGAATCCGGGGTCCCTGACCGATTCAGTGGC |
| | | AGCGGGTCTGGGACAGATTTCACTCTCACCATCAGCAGCCTGCAGGCTGAAGATGTGGCAGTTTATTACT |
| | | GTCAGCAGTTCTACCTCCCCCCTTGGACTTTTGGCGGAGGGACCAAGGTTGAGATCAAA |
| 273 | BIIB-9-622_VL | EIVMTQSPATLSVSPGERATLSCRASQSVSSSLAWYQQKPGQAPRLLIYGASTRATGIPARFSGSGSGTE |
| | Amino Acid | FTLTISSLQSEDFAVYYCQQHSTWPPTFGGGTKVEIK |
| | Sequence | |
| 274 | BIIB-9-622_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAGCTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGCACTCCACCT |
| | | GGCCTCCTACTTTTGGCGGAGGGACCAAGGTTGAGATCAAA |
| 275 | BIIB-9-627_VL | DIVMTQSPLSLPVTPGEPASISCRSSQSLLHSNGYNYLDWYLQKPGQSPQLLIYLGSNRASGVPDRFSGS |
| | Amino Acid | GSGTDFTLKISRVEAEDVGVYYCMQARERPWTFGGGTKVEIK |
| | Sequence | |
| 276 | BIIB-9-627_VL | GATATTGTGATGACTCAGTCTCCACTCTCCCTGCCCGTCACCCCTGGAGAGCCGGCCTCCATCTCCTGCA |
| | Nucleic Acid | GGTCTAGTCAGAGCCTCCTGCATAGTAATGGATACAACTATTTGGATTGGTACCTGCAGAAGCCAGGGCA |
| | Sequence | GTCTCCACAGCTCCTGATCTATTTGGGTTCTAATCGGGCCTCCGGGGTCCCTGACAGGTTCAGTGGCAGT |
| | | GGATCAGGCACAGATTTTACACTGAAAATCAGCAGAGTGGAGGCTGAGGATGTTGGGGTTTATTACTGCA |
| | | TGCAGGCACGAGAACGCCCTTGGACTTTTGGCGGAGGGACCAAGGTTGAGATCAAA |

Class II Antibodies _VL Sequences

| | | |
|---|---|---|
| 277 | BIIB-9-408_VL | EIVLTQSPATLSLSPGERATLSCRASQSVSSYLAWYQQKPGQAPRLLIYDASNRATGIPARFSGSGSGTD |
| | Amino Acid | FTLTISSLEPEDFAVYYCQQAFVWPPITFGGGTKVEIK |
| | Sequence | |
| 278 | BIIB-9-408_VL | GAAATTGTGTTGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGGCCTTCGTCT |
| | | GGCCTCCTATCACTTTTGGCGGAGGGACCAAGGTTGAGATCAAA |
| 279 | BIIB-9-416_VL | EIVMTQSPATLSLSPGERATLSCRASQSVSSYLAWYQQKPGQAPRLLIYDSSNRATGIPARFSGSGSGTD |
| | Amino Acid | FTLTISSLEPEDFAVYYCQQRVVWPPTFGGGTKVEIK |
| | Sequence | |
| 280 | BIIB-9-416_VL | GAAATTGTGATGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATTCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGAGAGTCGTCT |
| | | GGCCTCCTACTTTTGGCGGAGGGACCAAGGTTGAGATCAAA |
| 281 | BIIB-9-629_VL | DIQLTQSPSSLSASVGDRVTITCQASQDISNYLNWYQQKPGKAPKLLIYDASNLETGVPSRFSGSGSGTD |
| | Amino Acid | FTFTISSLQPEDIATYYCQQLDSLPPTFGGGTKVEIK |
| | Sequence | |
| 282 | BIIB-9-629_VL | GACATCCAGTTGACCCAGTCTCCATCCTCCCTGTCTGCATCTGTAGGAGACAGAGTCACCATCACTTGCC |
| | Nucleic Acid | AGGCGAGTCAGGACATTAGCAACTATTTAAATTGGTATCAGCAGAAACCAGGGAAAGCCCCTAAGCTCCT |
| | Sequence | GATCTACGATGCATCCAATTTGGAAACAGGGGTCCCATCAAGGTTCAGTGGAAGTGGATCTGGGACAGAT |
| | | TTTACTTTCACCATCAGCAGCCTGCAGCCTGAAGATATTGCAACATATTACTGTCAGCAGCTCGATTCCC |
| | | TCCCTCCTACTTTTGGCGGAGGGACCAAGGTTGAGATCAAA |
| 283 | BIIB-9-885_VL | EIVLTQSPATLSLSPGERATLSCRASQSVSSYLAWYQQKPGQAPRLLIYDASKRATGIPARFSGSGSGTD |
| | Amino Acid | FTLTISSLEPEDFAVYYCQQRVIWPPTFGGGTKVEIK |
| | Sequence | |
| 284 | BIIB-9-885_VL | GAAATTGTGTTGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATGCATCCAAAAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGAGAGTCATCT |
| | | GGCCTCCTACTTTTGGCGGAGGGACCAAGGTTGAGATCAAA |

Class III Antibodies _VL Sequences

| | | |
|---|---|---|
| 285 | BIIB-9-607_VL | DIQMTQSPSTLSASVGDRVTITCRASQSISSWLAWYQQKPGKAPKLLIYKASSLESGVPSRFSGSGSGTE |
| | Amino Acid | FTLTISSLQPDDFATYYCQQAGRYPLTFGGGTKVEIK |
| | Sequence | |
| 286 | BIIB-9-607_VL | GACATCCAGATGACCCAGTCTCCTTCCACCCTGTCTGCATCTGTAGGAGACAGAGTCACCATCACTTGCC |
| | Nucleic Acid | GGGCCAGTCAGAGTATTAGTAGCTGGTTGGCCTGGTATCAGCAGAAACCAGGGAAAGCCCCTAAGCTCCT |
| | Sequence | GATCTATAAAGCCTCCAGTTTGGAAAGTGGGGTCCCATCAAGGTTCAGCGGCAGTGGATCTGGGACAGAA |
| | | TTCACTCTCACCATCAGCAGCCTGCAGCCTGATGATTTTGCAACTTATTACTGCCAGCAGGCCGGACGCT |
| | | ACCCTCTCACTTTTGGCGGAGGGACCAAGGTTGAGATCAAA |
| 287 | BIIB-9-471_VL | EIVLTQSPATLSLSPGERATLSCRASQSVSRYLAWYQQKPGQAPRLLIYDASNRATGIPARFSGSGSGTD |
| | Amino Acid | FTLTISSLEPEDFAVYYCQQDYNYPFTFGGGTKVEIK |
| | Sequence | |
| 288 | BIIB-9-471_VL | GAAATTGTGTTGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGGTACTTAGCCTGGTACCAACAGAAACCTGGCCAGGCTCCCAGGCTCCT |
| | Sequence | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGGACTACAATT |
| | | ACCCTTTCACTTTTGGCGGAGGGACCAAGGTTGAGATCAAA |
| 289 | BIIB-9-472_VL | EIVLTQSPATLSLSPGERATLSCRASQSVSSYLAWYQQKPGQAPRLLIYDASNRATGIPARFSGSGSGTD |
| | Amino Acid | FTLTISSLEPEDFAVYYCQQRSDWPTFGGGTKVEIK |
| | Sequence | |
| 290 | BIIB-9-472_VL | GAAATTGTGTTGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGAGATCCGACT |
| | | GGCCTACTTTTGGCGGAGGGACCAAGGTTGAGATCAAA |
| 291 | BIIB-9-439_VL | EIVMTQSPATLSLSPGERATLSCRASQSVSSYLAWYQQKPGQAPRLLIYDSSNRATGIPARFSGSGSGTD |
| | Amino Acid | FTLTISSLEPEDFAVYYCQQRDNWPFTFGGGTKVEIK |
| | Sequence | |
| 292 | BIIB-9-439_VL | GAAATTGTGATGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATTCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGAGAGACAACT |
| | | GGCCTTTCACTTTTGGCGGAGGGACCAAGGTTGAGATCAAA |
| 293 | BIIB-9-446_VL | EIVLTQSPGTLSLSPGERATLSCRASQSVSSSYLAWYQQKPGQAPRLLIYGASRRATGIPDRFSGSGSGT |
| | Amino Acid | DFTLTISRLEPEDFAVYYCQQYGNSPLTFGGGTKVEIK |
| | Sequence | |
| 294 | BIIB-9-446_VL | GAAATTGTGTTGACGCAGTCTCCAGGCACCCTGTCTTTGTCTCCAGGGGAAAGAGCCACCCTCTCCTGCA |
| | Nucleic Acid | GGGCCAGTCAGAGTGTTAGCAGCAGCTACTTAGCCTGGTACCAGCAGAAACCTGGCCAGGCTCCCAGGCT |
| | Sequence | CCTCATCTATGGTGCATCCAGAAGGGCCACTGGCATCCCAGACAGGTTCAGTGGCAGTGGGTCTGGGACA |
| | | GACTTCACTCTCACCATCAGCAGACTGGAGCCTGAAGATTTTGCAGTGTATTACTGTCAGCAGTACGGAA |
| | | ACAGTCCTCTCACTTTTGGCGGAGGGACCAAGGTTGAGATCAAA |
| 295 | BIIB-9-568_VL | DIQMTQSPSSLSASVGDRVTITCQASQDISNYLNWYQQKPGKAPKLLIYDASNLETGVPSRFSGSGSGTD |
| | Amino Acid | FTFTISSLQPEDIATYYCQQYDDYLTFGGGTKVEIK |
| | Sequence | |
| 296 | BIIB-9-568_VL | GACATCCAGATGACCCAGTCTCCATCCTCCCTGTCTGCATCTGTAGGAGACAGAGTCACCATCACTTGCC |
| | Nucleic Acid | AGGCGAGTCAGGACATTAGCAACTATTTAAATTGGTATCAGCAGAAACCAGGGAAAGCCCCTAAGCTCCT |
| | Sequence | GATCTACGATGCATCCAATTTGGAAACAGGGGTCCCATCAAGGTTCAGTGGAAGTGGATCTGGGACAGAT |
| | | TTTACTTTCACCATCAGCAGCCTGCAGCCTGAAGATATTGCAACATATTACTGTCAGCAGTACGATGACT |
| | | ACCTCACTTTTGGCGGAGGGACCAAGGTTGAGATCAAA |
| 297 | BIIB-9-615_VL | EIVLTQSPATLSLSPGERATLSCRASQSVSSYLAWYQQKPGQAPRLLIYDASNRATGIPARFSGSGSGTD |
| | Amino Acid | FTLTISSLEPEDFAVYYCQQSCHWPWTFGGGTKVEIK |
| | Sequence | |
| 298 | BIIB-9-615_VL | GAAATTGTGTTGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGTCCTGTCACT |
| | | GGCCTTGGACTTTTGGCGGAGGGACCAAGGTTGAGATCAAA |
| 299 | BIIB-9-628_VL | EIVLTQSPGTLSLSPGERATLSCRASQSVSSDYLAWYQQKPGQAPRLLIYGASSRATGIPDRFSGSGSGT |
| | Amino Acid | DFTLTISRLEPEDFAVYYCQQYWFPFTFGGGTKVEIK |
| | Sequence | |
| 300 | BIIB-9-628_VL | GAAATTGTGTTGACGCAGTCTCCAGGCACCCTGTCTTTGTCTCCAGGGGAAAGAGCCACCCTCTCCTGCA |
| | Nucleic Acid | GGGCCAGTCAGAGTGTTAGCAGCGACTACTTAGCCTGGTACCAGCAGAAACCTGGCCAGGCTCCCAGGCT |
| | Sequence | CCTCATCTATGGTGCATCCAGCAGGGCCACTGGCATCCCAGACAGGTTCAGTGGCAGTGGGTCTGGGACA |
| | | GACTTCACTCTCACCATCAGCAGACTGGAGCCTGAAGATTTTGCAGTGTATTACTGTCAGCAGTACGTCG |
| | | TCTTCCCTTTCACTTTTGGCGGAGGGACCAAGGTTGAGATCAAA |
| 301 | BIIB-9-882_VL | EIVMTQSPATLSVSPGERATLSCRASQSVSSNLAWYQQKPGQAPRLLIFGASTRATGIPARFSGSGSGTE |
| | Amino Acid | FTLTISSLQSEDFAVYYCQQHDNFPPTFGGGTKVEIK |
| | Sequence | |
| 302 | BIIB-9-882_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTTTGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGCACGACAATT |
| | | TCCCTCCTACTTTTGGCGGAGGGACCAAGGTTGAGATCAAA |
| 303 | BIIB-9-884_VL | EIVLTQSPGTLSLSPGERATLSCRASQSVSSSYLAWYQQKPGQAPRLLIYGASSRATGIPDRFSGSGSGT |
| | Amino Acid | DFTLTISRLEPEDFAVYYCQQYHLLPPTFGGGTKVEIK |
| | Sequence | |
| 304 | BIIB-9-884_VL | GAAATTGTGTTGACGCAGTCTCCAGGCACCCTGTCTTTGTCTCCAGGGGAAAGAGCCACCCTCTCCTGCA |
| | Amino Acid | GGGCCAGTCAGAGTGTTAGCAGCAGCTACTTAGCCTGGTACCAGCAGAAACCTGGCCAGGCTCCCAGGCT |
| | Sequence | CCTCATCTATGGTGCATCCAGCAGGGCCACTGGCATCCCAGACAGGTTCAGTGGCAGTGGGTCTGGGACA |
| | | GACTTCACTCTCACCATCAGCAGACTGGAGCCTGAAGATTTTGCAGTGTATTACTGTCAGCAGTACCACC |
| | | TCCTCCCTCCTACTTTTGGCGGAGGGACCAAGGTTGAGATCAAA |
| 305 | BIIB-9-886_VL | DIQMTQSPSSVSASVGDRVTITCRASQGISSWLAWYQQKPGKAPKLLIYAASSLQSGVPSRFSGSGSGTD |
| | Amino Acid | FTLTISSLQPEDFATYYCQQASSFPFTFGGGTKVEIK |
| | Sequence | |
| 306 | BIIB-9-886_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGCATCCAGTT |
| | | TCCCTTTCACTTTTGGCGGAGGGACCAAGGTTGAGATCAAA |
| 307 | BIIB-9-887_VL | DIQMTQSPSSVSASVGDRVTITCRASQGISSWLAWYQQKPGKAPKLLIYAASSLQSGVPSRFSGSGSGTD |
| | Amino Acid | FTLTISSLQPEDFATYYCQQASSFPFTFGGGTKVEIK |
| | Sequence | |
| 308 | BIIB-9-887_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGCATCCAGTT |
| | | TCCCTTTCACTTTTGGCGGAGGGACCAAGGTTGAGATCAAA |
| 309 | BIIB-9-888_VL | EIVMTQSPATLSVSPGERATLSCRASQSVSSNLAWYQQKPGQAPRLLIYGASTRATGIPARFSGSGSGTE |
| | Amino Acid | FTLTISSLQPEDFAVYYCQQAFNWPPTFGGGTKVEIK |
| | Sequence | |
| 310 | BIIB-9-888_VL | GAAATAGTGATGACGCAGTCTCCAGCCACCCTGTCTGTGTCTCCAGGGGAAAGGGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGCCTGAAGATTTTGCAGTTTATTACTGTCAGCAGGCCTTCAACT |
| | | GGCCTCCTACTTTTGGCGGAGGGACCAAGGTTGAGATCAAA |
| 311 | BIIB-9-889_VL | EIVMTQSPATLSVSPGERATLSCRASQSVSSNLAWYQQKPGQAPRLLIYGASTRATGIPARFSGSGSGTE |
| | Amino Acid | FTLTISSLQSEDFAVYYCQQAFNWPPTFGGGTKVEIK |
| | Sequence | |
| 312 | BIIB-9-889_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATTAGCAGCCTGCAGTCTGAAGATTTTGCAGTTTATTACTGTCAGCAGGCCTTCAACT |
| | | GGCCTCCTACTTTTGGCGGAGGGACCAAGGTTGAGATCAAA |
| 313 | BIIB-9-433_VL | EIVLTQSPATLSLSPGERATLSCRASQSVSSYLAWYQQKPGQAPRLLIYDASNRATGIPARFSGSGSGTD |
| | Amino Acid | FTLTISSLEPEDFAVYYCQQSSAYPPTFGGGTKVEIK |
| | Sequence | |
| 314 | BIIB-9-433_VL | GAAATTGTGTTGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGTCCAGTGCCT |
| | | ACCCTCCTACTTTTGGCGGAGGGACCAAGGTTGAGATCAAA |
| 315 | BIIB-9-445_VL | EIVMTQSPATLSVSPGERATLSCRASQSVSSNLAWYQQKPGQAPRLLIYSASTRATGIPARFSGSGSGTE |
| | Amino Acid | FTLTISSLQSEDFAVYYCQQYDNFPFTFGGGTKVEIK |
| | Sequence | |
| 316 | BIIB-9-445_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATAGCGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGTACGACAATT |
| | | TCCCTTTCACTTTTGGCGGAGGGACCAAGGTTGAGATCAAA |
| 317 | BIIB-9-470_VL | DIQMTQSPSTLSASVGDRVTITCRASQSISSWLAWYQQKPGKAPKLLIYKASSLESGVPSRFSGSGSGTE |
| | Amino Acid | FTLTISSLQPDDFATYYCQHPHSWTFGGGTKVEIK |
| | Sequence | |
| 318 | BIIB-9-470_VL | GACATCCAGATGACCCAGTCTCCTTCCACCCTGTCTGCATCTGTAGGAGACAGAGTCACCATCACTTGCC |
| | Nucleic Acid | GGGCCAGTCAGAGTATTAGTAGCTGGTTGGCCTGGTATCAGCAGAAACCAGGGAAAGCCCCTAAGCTCCT |
| | Sequence | GATCTATAAAGCCTCCAGTTTGGAAAGTGGGGTCCCATCAAGGTTCAGCGGCAGTGGATCTGGGACAGAA |
| | | TTCACTCTCACCATCAGCAGCCTGCAGCCTGATGATTTTGCAACTTATTACTGCCAGCATCCCCACTCTT |
| | | GGACTTTTGGCGGAGGGACCAAGGTTGAGATCAAA |
| 319 | BIIB-9-625_VL | DIQMTQSPSSLSASVGDRVTITCRASQSISSYLNWYQQKPGKAPKLLIYAASSLQSGVPSRFSGSGSGTD |
| | Amino Acid | FTLTISSLQPEDFATYYCQQSDTDPPTFGGGTKVEIK |
| | Sequence | |
| 320 | BIIB-9-625_VL | GACATCCAGATGACCCAGTCTCCATCCTCCCTGTCTGCATCTGTAGGAGACAGAGTCACCATCACTTGCC |
| | Nucleic Acid | GGGCAAGTCAGAGCATTAGCAGCTATTTAAATTGGTATCAGCAGAAACCAGGGAAAGCCCCTAAGCTCCT |
| | Sequence | GATCTATGCTGCATCCAGTTTGCAAAGTGGGGTCCCATCAAGGTTCAGTGGCAGTGGATCTGGGACAGAT |
| | | TTCACTCTCACCATCAGCAGTCTGCAACCTGAAGATTTTGCAACTTACTACTGTCAGCAAAGCGACACCG |
| | | ACCCTCCTACTTTTGGCGGAGGGACCAAGGTTGAGATCAAA |
| 321 | BIIB-9-1264_VL | DIQMTQSPSSLSASVGDRVTITCQASQDITNYLNWYQQKPGKAPKLLIYDASNLETGVPSRFSGSGSGTD |
| | Amino Acid | FTFTISSLQPEDIATYYCQQVDDYPFTFGGGTKVEIK |
| | Sequence | |
| 322 | BIIB-9-1264_VL | GACATCCAGATGACCCAGTCTCCATCCTCCCTGTCTGCATCTGTAGGAGACAGAGTCACCATCACTTGCC |
| | Nucleic Acid | AGGCGAGTCAGGACATTACCAACTATTTAAATTGGTATCAGCAGAAACCAGGGAAAGCCCCTAAGCTCCT |
| | Sequence | GATCTACGATGCATCCAATTTGGAAACAGGGGTCCCATCAAGGTTCAGTGGAAGTGGATCTGGGACAGAT |
| | | TTTACTTTCACCATCAGCAGCCTGCAGCCTGAAGATATTGCAACATATTACTGTCAGCAGGTCGATGACT |
| | | ACCCTTTCACTTTTGGCGGAGGGACCAAGGTTGAGATCAAA |
| 323 | BIIB-9-1265_VL | DIQMTQSPSSVSASVGDRVTITCRASQGISSWLAWYQQKPGKAPKLLIYAASSLQSGVPSRFSGSGSGTD |
| | Amino Acid | FTLTISSLQPEDFATYYCQQGNSFPITFGGGTKVEIK |
| | Sequence | |
| 324 | BIIB-9-1265_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGGAAATAGTT |
| | | TCCCTATCACTTTTGGCGGAGGGACCAAGGTTGAGATCAAA |
| 325 | BIIB-9-1266_VL | EIVMTQSPATLSLSPGERATLSCRASQSVSSYLAWYQQKPGQAPRLLIYDSSNRATGIPARFSGSGSGTD |
| | Amino Acid | FTLTISSLEPEDFAVYYCQQRLNFPFTFGGGTKVEIK |
| | Sequence | |
| 326 | BIIB-9-1266_VL | GAAATTGTGATGACACAGTCTCCAGCCACCCTGTCTTTGTCCCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATTCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGAGACTCAATT |
| | | TCCCTTTCACTTTTGGCGGAGGGACCAAGGTTGAGATCAAA |
| 327 | BIIB-9-1267_VL | DIVMTQSPDSLAVSLGERATINCKSSQSVLYSSNNKNYLAWYQQKPGQPPKLLIYWASTRESGVPDRFSG |
| | Amino Acid | SGSGTDFTLTISSLQAEDVAVYYCQQHYVFPFTFGGGTKVEIK |
| | Sequence | |
| 328 | BIIB-9-1267_VL | GACATCGTGATGACCCAGTCTCCAGACTCCCTGGCTGTGTCTCTGGGCGAGAGGGCCACCATCAACTGCA |
| | Nucleic Acid | AGTCCAGCCAGAGTGTTTTATACAGCTCCAACAATAAGAACTACTTAGCTTGGTACCAGCAGAAACCAGG |
| | Sequence | ACAGCCTCCTAAGCTGCTCATTTACTGGGCATCTACCCGGGAATCCGGGGTCCCTGACCGATTCAGTGGC |
| | | AGCGGGTCTGGGACAGATTTCACTCTCACCATCAGCAGCCTGCAGGCTGAAGATGTGGCAGTTTATTACT |
| | | GTCAGCAGCACTACGTCTTCCCTTTCACTTTTGGCGGAGGGACCAAGGTTGAGATCAAA |
| 329 | BIIB-9-1268_VL | EIVMTQSPATLSLSPGERATLSCRASQSVSSYLAWYQQKPGQAPRLLIYDASNRATGIPARFSGSGSGTD |
| | Amino Acid | FTLTISSLEPEDFAVYYCQQATVWPFTFGGGTKVEIK |
| | Sequence | |
| 330 | BIIB-9-1268_VL | GAAATTGTGATGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGGCCACCGTCT |
| | | GGCCTTTCACTTTTGGCGGAGGGACCAAGGTTGAGATCAAA |
| 331 | BIIB-9-1269_VL | DIQMTQSPSSVSASVGDRVAITCRASQGISSWLAWYQQKPGKAPKLLIYAASSLQSGVPSRFSGSGSGTD |
| | Amino Acid | FTLTISSLQPEDFATYYCQQASSFPFTFGGGTKVEIK |
| | Sequence | |
| 332 | BIIB-9-1269_VL | GACATCCAGATGACCCAGTCTCCATCTTCCGTGTCTGCATCTGTAGGAGACAGAGTCGCCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGCATCCAGTT |
| | | TCCCTTTCACTTTTGGCGGAGGGACCAAGGTTGAGATCAAA |
| 333 | BIIB-9-1270_VL | EIVLTQSPATLSLSPGERATLSCRASQSVSSYLAWYQQKPGQAPRLLIYDASNRATGIPARFSGSGSGTD |
| | Amino Acid | FTLTISSLEPEDFAVYYCQQSADFPFTFGGGTKVEIK |
| | Sequence | |
| 334 | BIIB-9-1270_VL | GAAATTGTGTTGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGTCCGCCGATT |
| | | TCCCTTTCACTTTTGGCGGAGGGACCAAGGTTGAGATCAAA |
| 335 | BIIB-9-1271_VL | EIVMTQSPATLSVSPGERATLSCRASQSVSSNLAWYQQKPGQAPRLLIYGASTRATGIPARFSGSGSGTD |
| | Amino Acid | FTLTISSLQPEDFATYYCQQGFSFPFTFGGGTKVEIK |
| | Sequence | |
| 336 | BIIB-9-1271_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAT |
| | | TTCACTCTCACCATCAGCAGCCTGCAGCCTGAAGATTTTGCAACTTATTACTGTCAGCAGGGATTCAGTT |
| | | TCCCTTTCACTTTTGGCGGAGGGACCAAGGTTGAGATCAAA |
| 337 | BIIB-9-1272_VL | DIQMTQSPSSVSASVGDRVTITCRASQGISSWLAWYQQKPGKAPKLLIYAASSLQSGVPSRFSGSGSGTD |
| | Amino Acid | FTLTISSLQPEDFATYYCQQASSFPFTFGGGTKVEIK |
| | Sequence | |
| 338 | BIIB-9-1272_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGCATCCAGTT |
| | | TCCCTTTCACTTTTGGCGGAGGGACCAAGGTTGAGATCAAA |
| 339 | BIIB-9-1273_VL | EIVLTQYPATLSLSPGERATLSCRASQSVSSYLAWYQQKPGQAPRLLIYDASNRATGIPARFSGSGSGTD |
| | Amino Acid | FTLTISSLEPEDFAVYYCQQSANFPFTFGGGTKVEIK |
| | Sequence | |
| 340 | BIIB-9-1273_VL | GAAATTGTGTTGACACAGTATCCAGCCACCCTGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGTCCGCCAATT |
| | | TCCCTTTCACTTTTGGCGGAGGGACCAAGGTTGAGATCAAA |
| 341 | BIIB-9-1274_VL | DIQMTQSPSSVSASVGDRVTITCRASQGISSWLAWYQQKPGKAPKLLIYAASSLQSGVPSRFSGSGSGTD |
| | Amino Acid | FTLTISSLQPEDFATYYCQQANSFPFTFGGGTKVEIK |
| | Sequence | |
| 342 | BIIB-9-1274_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGCAAATTCCT |
| | | TCCCTTTCACTTTTGGCGGAGGGACCAAGGTTGAGATCAAA |
| 343 | BIIB-9-1275_VL | DIQMTQSPSSVSASVGDRVTITCRASQGISGWLAWYQQKPGKAPKLLIYAASSLQSGVPSRFSGSGSGTD |
| | Amino Acid | FTLTISSLQPEDFATYYCQQANSLPITFGGGTKVEIK |
| | Sequence | |
| 344 | BIIB-9-1275_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCGGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGCAAATTCCC |
| | | TCCCTATCACTTTTGGCGGAGGGACCAAGGTTGAGATCAAA |
| 345 | BIIB-9-1276_VL | DIQMTQSPSSVSASVGDRVTITCRASQGISSWLAWYQQKPGKAPKLLIYAASSLQSGVPSRFSGSGSGTD |
| | Amino Acid | FTLTISSLQPEDFATYYCQQGNSFPITFGGGTKVEIK |
| | Sequence | |
| 346 | BIIB-9-1276_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGGAAATAGTT |
| | | TCCCTATCACTTTTGGCGGAGGGACCAAGGTTGAGATCAAA |
| 347 | BIIB-9-1277_VL | EIVMTQSPATLSVSPGERATLSCRASQSVSSYLAWYQQKPGQAPRLLIYDASNRATGIPARFSGSGSGTD |
| | Amino Acid | FTLTISSLEPEDLAVYYCQQSANWPPTFGGGTKVEIK |
| | Sequence | |
| 348 | BIIB-9-1277_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATCTTGCAGTTTATTACTGTCAGCAGTCCGCCAATT |
| | | GGCCTCCTACTTTTGGCGGAGGGACCAAGGTTGAGATCAAA |
| 349 | BIIB-9-1278_VL | EIVMTQSPATLSVSPGERATLSCRASQSVSSNLAWYQQKPGQAPRLLIYGASTRATGIPARFSGSGSGTE |
| | Amino Acid | FTLTISSLQSEDFAVYYCQQHANFPPTFGGGTKVEIK |
| | Sequence | |
| 350 | BIIB-9-1278_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGCACGCCAATT |
| | | TTCCTCCTACTTTTGGCGGAGGGACCAAGGTTGAGATCAAA |
| 351 | BIIB-9-1279_VL | DIQMTQSPSSVSASVGDRVTITCRASQGISSWLAWYQQKPGKAPKLLIYAASSLQSGVPSRFSGSGSGTD |
| | Amino Acid | FTLTISSLQPEDFATYYCQQASSFPPTFGGGTKVEIK |
| | Sequence | |
| 352 | BIIB-9-1279_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGCATCCAGTT |
| | | TCCCTCCTACTTTTGGCGGAGGGACCAAGGTTGAGATCAAA |
| 353 | BIIB-9-1280_VL | DIQMTQSPSSLSASVGDRVTITCRASQSISSYLNWYQQKPGKAPKLLIYAASSLQSGVPSRFSGSGSGTD |
| | Amino Acid | FTLTISSLQPEDFATYYCQQAYSLPITFGGGTKVEIK |
| | Sequence | |
| 354 | BIIB-9-1280_VL | GACATCCAGATGACCCAGTCTCCATCCTCCCTGTCTGCATCTGTAGGAGACAGAGTCACCATCACTTGCC |
| | Nucleic Acid | GGGCAAGTCAGAGCATTAGCAGCTATTTAAATTGGTATCAGCAGAAACCAGGGAAAGCCCCTAAGCTCCT |
| | Sequence | GATCTATGCTGCATCCAGTTTGCAAAGTGGGGTCCCATCAAGGTTCAGTGGCAGTGGATCTGGGACAGAT |
| | | TTCACTCTCACCATCAGCAGTCTGCAACCTGAAGATTTTGCAACTTACTACTGTCAGCAAGCATACAGTC |
| | | TCCCTATCACTTTTGGCGGAGGGACCAAGGTTGAGATCAAA |
| 355 | BIIB-9-1281_VL | DIQMTQSPSTLSASVGDRVTITCRASQSISSWLAWYQQKPGKAPKLLIYKASSLESGVPSRFSGSGSGTD |
| | Amino Acid | FTLTISSLQPEDFATYYCQQAISLPITFGGGTKVEIK |
| | Sequence | |
| 356 | BIIB-9-1281_VL | GACATCCAGATGACCCAGTCTCCTTCCACCCTGTCTGCATCTGTAGGAGACAGAGTCACCATCACTTGCC |
| | Nucleic Acid | GGGCCAGTCAGAGTATTAGTAGCTGGTTGGCCTGGTATCAGCAGAAACCAGGGAAAGCCCCTAAGCTCCT |
| | Sequence | GATCTATAAAGCCTCCAGTTTGGAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGCAATCAGTC |
| | | TCCCTATCACTTTTGGCGGAGGGACCAAGGTTGAGATCAAA |
| 357 | BIIB-9-1282_VL | EIVMTQSPATLSVSPGERATLSCRASQSVSSNLAWYQQKPGQAPRLLIYGASTRATGIPARFSGSGSGTE |
| | Amino Acid | FTLTISSLQSEDFAVYYCQQHNHLPITFGGGTKVEIK |
| | Sequence | |
| 358 | BIIB-9-1282_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGCACAATCACC |
| | | TCCCTATCACTTTTGGCGGAGGGACCAAGGTTGAGATCAAA |
| 359 | BIIB-9-1283_VL | DIQMTQSPSSVSASVGDRVTITCRASQGISSWLAWYQQKPGKAPKLLIYAASSLQSGVPSRFSGSGSGTD |
| | Amino Acid | FTLTISSLQPEDFATYYCQQASSFPPTFGGGTKVEIK |
| | Sequence | |
| 360 | BIIB-9-1283_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGCATCCAGTT |
| | | TCCCTCCTACTTTTGGCGGAGGGACCAAGGTTGAGATCAAA |
| 361 | BIIB-9-1284_VL | EIVLTQSPATLSVSPGERATLSCRASQSVGSNLAWYQQKPGQAPRLLIYGASTRATGIPARFSGSGSGTE |
| | Amino Acid | FTLTISSLQSEDFAVYYCQQHNHLPITFGGGTKVEIK |
| | Sequence | |
| 362 | BIIB-9-1284_VL | GAAATAGTGTTGACGCAGTCTCCAGCCACCCTGTCTGTGTCTCCAGGGGAAAGAGCCACCCTCTCCTGCA |
| | Nucleic Acid | GGGCCAGTCAGAGTGTTGGCAGCAACTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACTATCAGCAGCCTGCAGTCTGAAGATTTTGCAGTTTATTACTGTCAGCAGCACAATCACC |
| | | TCCCTATCACTTTTGGCGGAGGGACCAAGGTTGAGATCAAA |
| 363 | BIIB-9-1285_VL | EIVMTQSPATLSVSPGERATLSCRASQSVSSNLAWYQQKPGQAPRLLIYGASTRATGIPARFSGSGSGTE |
| | Amino Acid | FTLTISSLQSEDFAVYYCQQASNFPPTFGGGTKVEIK |
| | Sequence | |
| 364 | BIIB-9-1285_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGGCCTCCAATT |
| | | TCCCTCCTACTTTTGGCGGAGGGACCAAGGTTGAGATCAAA |
| 365 | BIIB-9-1286_VL | DIQMTQSPSSVSASVGDRVTITCRASQGISSWLAWYQQKPGKAPKLLIYAASSLQSGVPSRFSGSGSGTD |
| | Amino Acid | FTLTISSLQPEDFVTYYCQQASSFPPTFGGGTKVEIK |
| | Sequence | |
| 366 | BIIB-9-1286_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGTAACTTATTACTGTCAGCAGGCATCCAGTT |
| | | TCCCTCCTACTTTTGGCGGAGGGACCAAGGTTGAGATCAAA |
| 367 | BIIB-9-1287_VL | EIVLTQSPGTLSLSPGERATLSCRASQSVSSSYLAWYQQKPGQAPRLLIYGASSRATGIPDRFSGSGSGT |
| | Amino Acid | DFTLTISRLEPEDFAVYYCQQYHLHPTFGGGTKVEIK |
| | Sequence | |
| 368 | BIIB-9-1287_VL | GAAATTGTGTTGACGCAGTCTCCAGGCACCCTGTCTTTGTCTCCAGGGGAAAGAGCCACCCTCTCCTGCA |
| | Nucleic Acid | GGGCCAGTCAGAGTGTTAGCAGCAGCTACTTAGCCTGGTACCAGCAGAAACCTGGCCAGGCTCCCAGGCT |
| | Sequence | CCTCATCTATGGTGCATCCAGCAGGGCCACTGGCATCCCAGACAGGTTCAGTGGCAGTGGGTCTGGGACA |
| | | GACTTCACTCTCACCATCAGCAGACTGGAGCCTGAAGATTTTGCAGTGTATTACTGTCAGCAGTACCACC |
| | | TCCACCCTACTTTTGGCGGAGGGACCAAGGTTGAGATCAAA |

Class IV Antibodies _VL Sequences

| | | |
|---|---|---|
| 369 | BIIB-9-397_VL | DIQMTQSPSSLSASVGDRVTITCQASQDISNYLNWYQQKPGKAPKLLIYDASNLETGVPSRFSGSGSGTD |
| | Amino Acid | FTFTISSLQPEDIATYYCQQSDDHPPTFGGGTKVEIK |
| | Sequence | |
| 370 | BIIB-9-397_VL | GACATCCAGATGACCCAGTCTCCATCCTCCCTGTCTGCATCTGTAGGAGACAGAGTCACCATCACTTGCC |
| | Nucleic Acid | AGGCGAGTCAGGACATTAGCAACTATTTAAATTGGTATCAGCAGAAACCAGGGAAAGCCCCTAAGCTCCT |
| | Sequence | GATCTACGATGCATCCAATTTGGAAACAGGGGTCCCATCAAGGTTCAGTGGAAGTGGATCTGGGACAGAT |
| | | TTTACTTTCACCATCAGCAGCCTGCAGCCTGAAGATATTGCAACATATTACTGTCAGCAGTCCGATGACC |
| | | ACCCTCCTACTTTTGGCGGAGGGACCAAGGTTGAGATCAAA |
| 371 | BIIB-9-578_VL | DIQMTQSPSSVSASVGDRVTITCRASQGIDSWLAWYQQKPGKAPKLLIYAASSLQSGVPSRFSGSGSGTD |
| | Amino Acid | FTLTISSLQPEDFATYYCQQANFLPFTFGGGTKVEIK |
| | Sequence | |
| 372 | BIIB-9-578_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTGACAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGCAAATTTCC |
| | | TCCCTTTCACTTTTGGCGGAGGGACCAAGGTTGAGATCAAA |
| 373 | BIIB-9-631_VL | DIQMTQSPSSVSASVGDRVTITCRASQGISRWLAWYQQKPGKAPKLLIYAASSLQSGVPSRFSGSGSGTD |
| | Amino Acid | FTLTISSLQPEDFATYYCQQRTSFPLTFGGGTKVEIK |
| | Sequence | |
| 374 | BIIB-9-631_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCAGGTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGAGAACCAGTT |
| | | TCCCTCTCACTTTTGGCGGAGGGACCAAGGTTGAGATCAAA |
| 375 | BIIB-9-612_VL | EIVLTQSPATLSLSPGERATLSCRASQSVSRYLAWYQQKPGQAPRLLIYDASNRATGIPARFSGSGSGTD |
| | Amino Acid | FTLTISSLEPEDFAVYYCQQSSLFPLTFGGGTKVEIK |
| | Sequence | |
| 376 | BIIB-9-612_VL | GAAATTGTGTTGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGGTACTTAGCCTGGTACCAACAGAAACCTGGCCAGGCTCCCAGGCTCCT |
| | Sequence | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGTCCAGTCTCT |
| | | TCCCTCTCACTTTTGGCGGAGGGACCAAGGTTGAGATCAAA |

Class V Antibody - VH Sequences

| | | |
|---|---|---|
| 377 | BIIB-12-891_VH | QVQLVQSGAEVKKPGASVKVSCKASGYTFTSYYIHWVRQAPGQGLEWMGIINPSGGSTSYAQKFQGRVTM |
| | Amino Acid | TRDTSTSTVYMELSSLRSEDTAVYYCARASIYRGLGAFDIWGQGTMVTVSS |
| | Sequence | |
| 378 | BIIB-12-891_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATCCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCTAGGGCATCTATATACCGAGGTCTCGGAGCCTTCGACATATGGGGTCAGGGTACAATGGT |
| | | CACCGTCTCCTCA |
| 379 | BIIB-12-892_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARGLGRRTPTAFDIWGQGTMVTVSS |
| | Sequence | |
| 380 | BIIB-12-892_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGGACTAGGAAGAAGAACTCCAACCGCCTTTGATATTTGGGGTCAGGGTACAATGGT |
| | | CACCGTCTCCTCA |
| 381 | BIIB-12-893_VH | QVQLVQSGAEVKKPGASVKVSCKASGYTFTSYYMVWVRQAPGQGLEWMGIINPSGGSTSYAQKFQGRVTM |
| | Amino Acid | TRDTSTSTVYMELSSLRSEDTAVYYCARDPGRRQYSFYGMDVWGQGTTVTVSS |
| | Sequence | |
| 382 | BIIB-12-893_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGGTCTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGATCCAGGAAGAAGGCAATACTCTTTCTACGGTATGGATGTCTGGGGCCAGGGAAC |
| | | AACTGTCACCGTCTCCTCA |
| 383 | BIIB-12-895_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARGGGYKSRGIDYWGQGTLVTVSS |
| | Sequence | |
| 384 | BIIB-12-895_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGGTGGAGGATACAAATCTAGAGGCATTGACTACTGGGGACAGGGTACATTGGTCAC |
| | | CGTCTCCTCA |
| 385 | BIIB-12-896_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARGLGQQRRGFDIWGQGTLVTVSS |
| | Sequence | |
| 386 | BIIB-12-896_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGGACTAGGACAGCAGCGGCGTGGCTTCGACATATGGGGTCAGGGTACATTGGTCAC |
| | | CGTCTCCTCA |
| 387 | BIIB-12-897_VH | QLQLQESGPGLVKPSETLSLTCTVSGGSISSSSYYWGWIRQPPGKGLEWIGSIYYSGSTYYNPSLKSRVT |
| | Amino Acid | ISVDTSKNQFSLKLSSVTAADTAVYYCAREGRTYYGGWFDPWGQGTLVTVSS |
| | Sequence | |
| 388 | BIIB-12-897_VH | CAGCTGCAGCTGCAGGAGTCGGGCCCAGGACTGGTGAAGCCTTCGGAGACCCTGTCCCTCACCTGCACTG |
| | Nucleic Acid | TCTCTGGTGGCTCCATCAGCAGTAGTAGTTACTACTGGGGCTGGATCCGCCAGCCCCCAGGGAAGGGGCT |
| | Sequence | GGAGTGGATTGGGAGTATCTATTATAGTGGGAGCACCTACTACAACCCGTCCCTCAAGAGTCGAGTCACC |
| | | ATATCCGTAGACACGTCCAAGAACCAGTTCTCCCTGAAGCTGAGTTCTGTGACCGCCGCAGACACGGCGG |
| | | TGTACTACTGCGCCAGAGAAGGGAGAACATACTACGGCGGTTGGTTCGATCCCTGGGGACAGGGTACATT |
| | | GGTCACCGTCTCCTCA |
| 389 | BIIB-12-898_VH | QVQLVQSGAEVKKPGASVKVSCKASGYTFTSYYMHWVRQAPGQGLEWMGIINPSGGSTTYAQKFQGRVTM |
| | Amino Acid | TRDTSTSTVYMELSSLRSEDTAVYYCARGDRMLRAFDPWGQGTLVTVSS |
| | Sequence | |
| 390 | BIIB-12-898_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAACCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGGGGACAGGATGTTAAGAGCATTCGACCCATGGGGACAGGGTACATTGGTCACCGT |
| | | CTCCTCA |
| 391 | BIIB-12-899_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARGPRTSSPLYFDLWGRGTLVTVSS |
| | Sequence | |
| 392 | BIIB-12-899_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGGGCCTAGAACATCATCACCTCTATACTTCGACCTATGGGGGAGAGGTACCTTGGT |
| | | CACCGTCTCCTCA |
| 393 | BIIB-12-900_VH | QVQLVQSGAEVKKPGASVKVSCKASGYTFTSYYMHWVRQAPGQGLEWMGIINPGGGSTSYAQKFQGRVTM |
| | Amino Acid | TRDTSTSTVYMELSSLRSEDTAVYYCARSGGMYDRELGMDVWGQGTTVTVSS |
| | Sequence | |
| 394 | BIIB-12-900_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTGGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGATCAGGCGGAATGTACGACCGAGAGCTCGGAATGGACGTATGGGGCCAGGGAACAAC |
| | | TGTCACCGTCTCCTCA |
| 395 | BIIB-12-901_VH | QVQLVQSGAEVKKPGASVKVSCKASGYTFTSYYMSWVRQAPGQGLEWMGIINPSGGSTSYAQKFQGRVTM |
| | Amino Acid | TRDTSTSTVYMELSSLRSEDTAVYYCARSRPRRPSPYGMDVWGQGTTVTVSS |
| | Sequence | |
| 396 | BIIB-12-901_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGTCATGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCTAGATCAAGACCAAGACGACCAAGCCCATACGGAATGGACGTATGGGGCCAGGGAACAAC |
| | | TGTCACCGTCTCCTCA |
| 397 | BIIB-12-902_VH | QVQLVQSGAEVKKPGASVKVSCKASGYTFTSYYMHWVRQAPGQGLEWMGIINPGGGSTSYAQKFQGRVTM |
| | Amino Acid | TRDTSTSTVYMELSSLRSEDTAVYYCARGGPRRAYSWYFDYWGQGTLVTVSS |
| | Sequence | |
| 398 | BIIB-12-902_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTGGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGGTGGGCCTAGAAGGGCCTACAGCTGGTACTTTGACTACTGGGGACAGGGTACATT |
| | | GGTCACCGTCTCCTCA |
| 399 | BIIB-12-903_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARDLGYTAGAFGYWGQGTLVTVSS |
| | Sequence | |
| 400 | BIIB-12-903_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGACCTGGGATACACCGCAGGGGCTTTTGGCTACTGGGGACAGGGTACATTGGTCAC |
| | | CGTCTCCTCA |
| 401 | BIIB-12-904_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCARGSGRSGYHYWGQGTLVTVSS |
| | Sequence | |
| 402 | BIIB-12-904_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAGAGGCTCTGGAAGATCCGGGTACCATTACTGGGGACAGGGTACATTGGTCACCGTCTC |
| | | CTCA |
| 403 | BIIB-12-905_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCAKGGTYLDTWGQGTLVTVSS |
| | Sequence | |
| 404 | BIIB-12-905_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAAGGGCGGAACATACTTAGACACTTGGGGACAGGGTACATTGGTCACCGTCTCCTCA |
| 405 | BIIB-12-906_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCAKSSRHFDYWGRGTLVTVSS |
| | Sequence | |
| 406 | BIIB-12-906_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAAGTCTAGTAGACATTTCGATTACTGGGGACGGGGTACATTGGTCACCGTCTCCTCA |
| 407 | BIIB-12-907_VH | QVQLQESGPGLVKPSETLSLTCAVSGYSISSGYYWAWIRQPPGKGLEWIGSIYHSGSTYYNPSLKSRVTI |
| | Amino Acid | SVDTSKNQFSLKLSSVTAADTAVYYCARESGMSGAAYWGQGTLVTVSS |
| | Sequence | |
| 408 | BIIB-12-907_VH | CAGGTGCAGCTGCAGGAGTCGGGCCCAGGACTGGTGAAGCCTTCGGAGACCCTGTCCCTCACCTGCGCTG |
| | Nucleic Acid | TCTCTGGTTACTCCATCAGCAGTGGTTACTACTGGGCTTGGATCCGGCAGCCCCCAGGGAAGGGGCTGGA |
| | Sequence | GTGGATTGGGAGTATCTATCATAGTGGGAGCACCTACTACAACCCGTCCCTCAAGAGTCGAGTCACCATA |
| | | TCAGTAGACACGTCCAAGAACCAGTTCTCCCTGAAGCTGAGTTCTGTGACCGCCGCAGACACGGCGGTGT |
| | | ACTACTGCGCCAGAGAGTCTGGAATGAGCGGAGCGGCTTACTGGGGACAGGGTACATTGGTCACCGTCTC |
| | | CTCA |
| 409 | BIIB-12-908_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCAKGPRGMDVWGQGTTVTVSS |
| | Sequence | |
| 410 | BIIB-12-908_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAAGGGCCCCAGAGGAATGGACGTATGGGGCCAGGGAACAACTGTCACCGTCTCCTCA |
| 411 | BIIB-12-909_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCAKGKHRRRSFDIWGQGTMVTVSS |
| | Sequence | |
| 412 | BIIB-12-909_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCAAAGGGCAAGCACAGAAGGAGGTCATTCGACATATGGGGTCAGGGTACAATGGTCACCGT |
| | | CTCCTCA |
| 413 | BIIB-12-910_VH | QVQLQESGPGLVKPSQTLSLTCTVSGGSISSGGYYWSWIRQHPGKGLEWIGSIYYSGSTYYNPSLKSRVT |
| | Amino Acid | ISVDTSKNQFSLKLSSVTAADTAVYYCARGPLQRQVRYFDLWGRGTLVTVSS |
| | Sequence | |
| 414 | BIIB-12-910_VH | CAGGTGCAGCTGCAGGAGTCGGGCCCAGGACTGGTGAAGCCTTCACAGACCCTGTCCCTCACCTGTACTG |
| | Nucleic Acid | TCTCTGGTGGCTCCATCAGCAGTGGTGGTTACTACTGGAGCTGGATCCGCCAGCACCCAGGGAAGGGCCT |
| | Sequence | GGAGTGGATTGGGTCAATCTATTACAGTGGGAGCACCTACTACAACCCGTCCCTCAAGAGTCGAGTTACC |
| | | ATATCAGTAGACACGTCTAAGAACCAGTTCTCCCTGAAGCTGAGTTCTGTGACCGCCGCAGACACGGCGG |
| | | TGTACTACTGCGCCAGAGGTCCCTTGCAAAGACAAGTGAGATACTTCGACCTATGGGGGAGAGGTACCTT |
| | | GGTCACCGTCTCCTCA |
| 415 | BIIB-12-911_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCARGRGMDVWGQGTTVTVSS |
| | Sequence | |
| 416 | BIIB-12-911_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAGAGGCAGGGGAATGGATGTATGGGGCCAGGGAACAACTGTCACCGTCTCCTCA |
| 417 | BIIB-12-912_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCAKSGGRYGYDIWGQGTMVTVSS |
| | Sequence | |
| 418 | BIIB-12-912_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAAGTCCGGTGGAAGATACGGGTACGACATATGGGGTCAGGGTACAATGGTCACCGTCTC |
| | | CTCA |
| 419 | BIIB-12-913_VH | QVQLVQSGAEVKKPGASVKVSCKASGYTFTSYGISWVRQAPGQGLEWMGWISAYNGNTNYAQKLQGRVTM |
| | Amino Acid | TTDTSTSTAYMELRSLRSDDTAVYYCARGGVSRFWGQGTLVTVSS |
| | Sequence | |
| 420 | BIIB-12-913_VH | CAGGTTCAGCTGGTGCAGTCTGGAGCTGAGGTGAAGAAGCCTGGGGCCTCAGTGAAGGTCTCCTGCAAGG |
| | Nucleic Acid | CTTCTGGTTACACCTTTACCAGCTATGGTATCAGCTGGGTGCGACAGGCCCCTGGACAAGGGCTTGAGTG |
| | Sequence | GATGGGATGGATCAGCGCTTACAATGGTAACACAAACTATGCACAGAAGCTCCAGGGCAGAGTCACCATG |
| | | ACCACAGACACATCCACGAGCACAGCCTACATGGAGCTGAGGAGCCTGAGATCTGACGACACGGCGGTGT |
| | | ACTACTGCGCCAGAGGTGGCGTGAGTAGATTCTGGGGACAGGGTACATTGGTCACCGTCTCCTCA |
| 421 | BIB-12-914_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARAPRYRGTMDVWGQGTTVTVSS |
| | Sequence | |
| 422 | BIIB-12-914_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGCCCCTAGATACCGAGGTACCATGGATGTGTGGGGCCAGGGAACAACTGTCACCGT |
| | | CTCCTCA |
| 423 | BIIB-12-915_VH | QLQLQESGPGLVKPSETLSLTCTVSGGSISSSRYYWGWIRQPPGKGLEWIGSIYYSGSTYYNPSLKSRVT |
| | Amino Acid | ISVDTSKNQFSLKLSSVTAADTAVYYCARVGGGYANPWGQGTLVTVSS |
| | Sequence | |
| 424 | BIIB-12-915_VH | CAGCTGCAGCTGCAGGAGTCGGGCCCAGGACTGGTGAAGCCTTCGGAGACCCTGTCCCTCACCTGCACTG |
| | Nucleic Acid | TCTCTGGTGGCTCCATCAGCAGTAGTCGCTACTACTGGGGCTGGATCCGCCAGCCCCCAGGGAAGGGGCT |
| | Sequence | GGAGTGGATTGGGAGTATCTATTATAGTGGGAGCACCTACTACAACCCGTCCCTCAAGAGTCGAGTCACC |
| | | ATATCCGTAGACACGTCCAAGAACCAGTTCTCCCTGAAGCTGAGTTCTGTGACCGCCGCAGACACGGCGG |
| | | TGTACTACTGCGCCAGAGTTGGAGGAGGATACGCCAACCCATGGGGACAGGGTACATTGGTCACCGTCTC |
| | | CTCA |
| 425 | BIIB-12-916_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARGGRQKATRRVDVWGQGTVVTVSS |
| | Sequence | |
| 426 | BIIB-12-916_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGGAGGTAGACAAAAGGCAACAAGGAGGGTAGACGTATGGGGTCAGGGTACAGTGGT |
| | | CACCGTCTCCTCA |
| 427 | BIIB-12-917_VH | EVQLVQSGAEVKKPGESLKISCKGSGYSFTTYWIGWVRQMPGKGLEWMGIIYPGDSDTRYSPSFQGQVTI |
| | Amino Acid | SADKSISTAYLQWSSLKASDTAMYYCARGRFRPRGRFDYWGQGTLVTVSS |
| | Sequence | |
| 428 | BIIB-12-917_VH | GAGGTGCAGCTGGTGCAGTCTGGAGCAGAGGTGAAAAAGCCCGGGGAGTCTCTGAAGATCTCCTGTAAGG |
| | Nucleic Acid | GTTCTGGATACAGCTTTACCACCTACTGGATCGGCTGGGTGCGCCAGATGCCCGGGAAAGGCCTGGAGTG |
| | Sequence | GATGGGGATCATCTATCCTGGTGACTCTGATACCAGATACAGCCCGTCCTTCCAAGGCCAGGTCACCATC |
| | | TCAGCCGACAAGTCCATCAGCACCGCCTACCTGCAGTGGAGCAGCCTGAAGGCCTCGGACACGGCGATGT |
| | | ACTACTGCGCCAGAGGCAGATTCAGACCTAGAGGCAGATTCGACTACTGGGGACAGGGTACATTGGTCAC |
| | | CGTCTCCTCA |
| 429 | BIIB-12-918_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCAKSLGGRSFDIWGQGTMVTVSS |
| | Sequence | |
| 430 | BIIB-12-918_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCAAAGAGCTTGGGAGGTAGATCATTCGACATATGGGGTCAGGGTACAATGGTCACCGTCTC |
| | | CTCA |
| 431 | BIIB-12-919_VH | QVQLVQSGAEVKKPGASVKVSCKASGYTFTSYYMHWVRQAPGQGLEWMGVINPSGGSTSYAQKFQGRVTM |
| | Amino Acid | TRDTSTSTVYMELSSLRSEDTAVYYCARGANIAVGRRYADYWGQGTLVTVSS |
| | Sequence | |
| 432 | BIIB-12-919_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAGTCATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGGTGCTAACATAGCAGTCGGCAGACGCTACGCAGACTACTGGGGACAGGGTACATT |
| | | GGTCACCGTCTCCTCA |
| 433 | BIIB-12-920_VH | QVQLVQSGAEVKKPGASVKVSCKASGYTFTSYGISWVRQAPGQGLEWMGWISPYNGNTNYAQKLQGRVTM |
| | Amino Acid | TTDTSTSTAYMELRSLRSDDTAVYYCARGSGHTTMFWGQGTLVTVSS |
| | Sequence | |
| 434 | BIIB-12-920_VH | CAGGTTCAGCTGGTGCAGTCTGGAGCTGAGGTGAAGAAGCCTGGGGCCTCAGTGAAGGTCTCCTGCAAGG |
| | Nucleic Acid | CTTCTGGTTACACCTTTACCAGCTATGGTATCAGCTGGGTGCGACAGGCCCCTGGACAAGGGCTTGAGTG |
| | Sequence | GATGGGATGGATCAGCCCTTACAATGGTAACACAAACTATGCACAGAAGCTCCAGGGCAGAGTCACCATG |
| | | ACCACAGACACATCCACGAGCACAGCCTACATGGAGCTGAGGAGCCTGAGATCTGACGACACGGCGGTGT |
| | | ACTACTGCGCCAGAGGGTCTGGACACACAACCATGTTCTGGGGACAGGGTACATTGGTCACCGTCTCCTC |
| | | A |
| 435 | BIIB-12-921_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCAKVGDRGTRAFDPWGQGTLVTVSS |
| | Sequence | |
| 436 | BIIB-12-921_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAAGGTAGGAGACAGAGGTACCCGTGCATTCGACCCATGGGGACAGGGTACATTGGTCAC |
| | | CGTCTCCTCA |
| 437 | BIIB-12-922_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARRGRIAGMWGQGTTVTVSS |
| | Sequence | |
| 438 | BIIB-12-922_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAAGAGGACGCATAGCAGGCATGTGGGGCCAGGGAACAACTGTCACCGTCTCCTCA |
| 439 | BIIB-12-923_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARGAGYLQRAFDIWGQGTMVTVSS |
| | Sequence | |
| 440 | BIIB-12-923_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGGTGCCGGATATCTACAGAGAGCCTTCGATATATGGGGTCAGGGTACAATGGTCAC |
| | | CGTCTCCTCA |
| 441 | BIIB-12-924_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCAKGKSSERGLNPWGQGTLVTVSS |
| | Sequence | |
| 442 | BIIB-12-924_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCAAAGGGCAAGAGCTCGGAACGAGGTCTCAACCCATGGGGACAGGGTACATTGGTCACCGT |
| | | CTCCTCA |
| 443 | BIIB-12-926_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARDGRMARGASPDYWGQGTLVTVSS |
| | Sequence | |
| 444 | BIIB-12-926_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGACGGCAGAATGGCAAGAGGTGCTAGCCCAGATTACTGGGGACAGGGTACATTGGT |
| | | CACCGTCTCCTCA |
| 445 | BIIB-12-927_VH | QVQLVQSGAEVKKPGASVKVSCKASGYTFTSYYMSWVRQAPGQGLEWMGIINPSGGSTSYAQKFQGRVTM |
| | Amino Acid | TRDTSTSTVYMELSSLRSEDTAVYYCARDSGRKRYYYMDVWGKGTTVTVSS |
| | Sequence | |
| 446 | BIIB-12-927_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGTCATGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGACTCAGGAAGGAAAAGATACTACTACATGGACGTATGGGGCAAGGGTACAACTGT |
| | | CACCGTCTCCTCA |
| 447 | BIIB-12-928_VH | QVQLVEFGVGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCAKGGTYLDTWGQGTLVTVSS |
| | Sequence | |
| 448 | BIIB-12-928_VH | CAGGTGCAGCTGGTGGAGTTTGGGGTAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAAGGGCGGAACATACTTAGACACTTGGGGACAGGGTACATTGGTCACCGTCTCCTCA ( |
| 449 | BIIB-12-929_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARDQGYSRSFDIWGQGTMVTVSS |
| | Sequence | |
| 450 | BIIB-12-929_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGGGACCAGGGATACTCAAGGTCATTCGACATATGGGGTCAGGGTACAATGGTCACCGT |
| | | CTCCTCA |
| 451 | BIIB-12-930_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARVRQKATLLFQHWGQGTLVTVSS |
| | Sequence | |
| 452 | BIIB-12-930_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGGGTCAGACAAAAGGCAACATTGTTGTTCCAACACTGGGGACAGGGTACATTGGTCAC |
| | | CGTCTCCTCA |
| 453 | BIIB-12-931_VH | QVQLVQSGAEVKKPGASVKVSCKASGYTFTSYYMSWVRQAPGQGLEWMGIINPSGGSTSYAQKFQGRVTM |
| | Amino Acid | TRDTSTSTVYMELSSLRSEDTAVYYCARDPVQEYGPYYYYMDVWGKGTTVTVSS |
| | Sequence | |
| 454 | BIIB-12-931_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGTCATGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGATCCGGTGCAGGAGTACGGCCCCTACTACTACTACATGGACGTATGGGGCAAGGG |
| | | TACAACTGTCACCGTCTCCTCA |
| 455 | BIIB-12-932_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCARLGYRGASAFDIWGQGTMVTVSS |
| | Sequence | |
| 456 | BIIB-12-932_VH | CAGGTGCAGCTGGTGGAGTCTGGGGGAGGCGTGGTCCAGCCTGGGAGGTCCCTGAGACTCTCCTGTGCAG |
| | Amino Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAGATTGGGATACAGAGGGGCCTCAGCTTTCGACATATGGGGTCAGGGTACAATGGTCAC |
| | | CGTCTCCTCA |
| 457 | BIIB-12-933_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCARLPRFTGTAYWGQGTLVTVSS |
| | Sequence | |
| 458 | BIIB-12-933_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAGACTTCCTAGATTCACTGGTACCGCTTACTGGGGACAGGGTACATTGGTCACCGTCTC |
| | | CTCA |
| 459 | BIIB-12-934_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCARESGGHSYGMDVWGQGTTVTVSS |
| | Sequence | |
| 460 | BIIB-12-934_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAGAGAGTCTGGAGGTCACAGCTACGGAATGGACGTATGGGGCCAGGGAACAACTGTCAC |
| | | CGTCTCCTCA |
| 461 | BIIB-12-935_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCARELEYGHYGMDVWGQGTTVTVSS |
| | Sequence | |
| 462 | BIIB-12-935_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAGAGAGTTGGAATATGGGCATTACGGAATGGACGTATGGGGCCAGGGAACAACTGTCAC |
| | | CGTCTCCTCA |
| 463 | BIIB-12-936_VH | EVQLLESGGGLVQPGGSLRLSCAASGFTFSSYAMSWVRQAPGKGLEWVSAISGSGGSTYYADSVKGRFTI |
| | Amino Acid | SRDNSKNTLYLQMNSLRAEDTAVYYCAKDGGWYEAYGMDVWGQGTTVTVSS |
| | Sequence | |
| 464 | BIIB-12-936_VH | GAGGTGCAGCTGTTGGAGTCTGGGGGAGGCTTGGTACAGCCTGGGGGGTCCCTGAGACTCTCCTGTGCAG |
| | Nucleic Acid | CCTCTGGATTCACCTTTAGCAGCTATGCCATGAGCTGGGTCCGCCAGGCTCCAGGGAAGGGGCTGGAGTG |
| | Sequence | GGTCTCAGCTATTAGTGGTAGTGGTGGTAGCACATACTACGCAGACTCCGTGAAGGGCCGGTTCACCATC |
| | | TCCAGAGACAATTCCAAGAACACGCTGTATCTGCAAATGAACAGCCTGAGAGCCGAGGACACGGCGGTGT |
| | | ACTACTGCGCCAAGGACGGTGGATGGTACGAGGCATACGGAATGGACGTATGGGGCCAGGGAACAACTGT |
| | | CACCGTCTCCTCA |
| 465 | BIIB-12-937_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARPTRMLRSYGMDVWGQGTTVTVSS |
| | Sequence | |
| 466 | BIIB-12-937_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGACCTACTAGGATGTTAAGGAGCTACGGAATGGACGTATGGGGCCAGGGAACAACTGT |
| | | CACCGTCTCCTCA |
| 467 | BIIB-12-1288_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARGGANDYGSSSRWWYFDLWGRGTLVTVSS |
| | Sequence | |
| 468 | BIIB-12-1288_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGGAGGTGCTAACGACTACGGCAGCAGCAGCCGATGGTGGTACTTCGACTTATGGGG |
| | | GAGAGGTACCTTGGTCACCGTCTCCTCA |
| 469 | BIIB-12-1289_VH | QVQLVQSGAEVKKPGASVKVSCKASGYTFTSYYMHWVRQAPGQGLEWMGIINPGGGSTSYAQKFQGRVTM |
| | Amino Acid | TRDTSTSTVYMELSSLRSEDTAVYYCARGPPRTYATGSHNWFDPWGQGTLVTVSS |
| | Sequence | |
| 470 | BIIB-12-1289_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTGGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGGACCGCCTAGAACCTACGCAACCGGAAGCCACAATTGGTTCGACCCCTGGGGACA |
| | | GGGTACATTGGTCACCGTCTCCTCA |
| 471 | BIIB-12-1290_VH | QVQLVQSGAEVKKPGASVKVSCKASGYTFTSYYMHWVRQAPGQGLEWMGIINPGGGSTSYAQKFQGRVTM |
| | Amino Acid | TRDTSTSTVYMELSSLRSEDTAVYYCAREGGRYVRGMDVWGQGTTVTVSS |
| | Sequence | |
| 472 | BIIB-12-1290_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTGGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGAGGGAGGAAGATACGTGAGAGGAATGGACGTATGGGGCCAGGGAACAACTGTCAC |
| | | CGTCTCCTCA |
| 473 | BIIB-12-1291_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARSRRMWVGYFDLWGRGTLVTVSS |
| | Sequence | |
| 474 | BIIB-12-1291_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCTAGATCAAGAAGGATGTGGGTAGGCTACTTCGACCTATGGGGGAGAGGTACCTTGGTCAC |
| | | CGTCTCCTCA |
| 475 | BIIB-12-1292_VH | QVQLVQSGAEVKKPGASVKVSCKASGYTFTSYYIHWVRQAPGQGLEWMGIINPSGGSTSYAQKFQGRVTM |
| | Amino Acid | TRDTSTSTVYMELSSLRSEDTAVYYCARDPGRRQSSGFDYWGQGTLVTVSS |
| | Sequence | |
| 476 | BIIB-12-1292_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATCCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGATCCAGGAAGAAGGCAAAGTTCTGGATTCGATTACTGGGGACAGGGTACATTGGT |
| | | CACCGTCTCCTCA |
| 477 | BIIB-12-1293_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARGSGGKHRGLDVWGQGTMVTVSS |
| | Sequence | |
| 478 | BIIB-12-1293_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGGCTCTGGAGGCAAACACAGAGGTCTAGACGTATGGGGTCAGGGTACAATGGTCAC |
| | | CGTCTCCTCA |
| 479 | BIIB-12-1294_VH | QVQLVQSGAEVKKPGASVKVSCKASGYTFTSYYMHWVRQAPGQGLEWMGIINPSGGSTSYAQKFQGRVTM |
| | Amino Acid | TRDTSTSTVYMELSSLRSEDTAVYYCARGTRSSRDMDVWGQGTTVTVSS |
| | Sequence | |
| 480 | BIIB-12-1294_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGGCACTAGATCCAGCAGAGACATGGATGTGTGGGGCCAGGGAACAACTGTCACCGT |
| | | CTCCTCA |
| 481 | BIIB-12-1295_VH | QVQLVQSGAEVKKPGASVKVSCKASGYTFTSYYMVWVRQAPGQGLEWMGIINPSGGSTSYAQKFQGRVTM |
| | Amino Acid | TRDTSTSTVYMELSSLRSEDTAVYYCARVGGATTGKIGYGMDVWGQGTTVTVSS |
| | Sequence | |
| 482 | BIIB-12-1295_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGGTCTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGTTGGAGGAGCCACCACAGGGAAAATCGGATACGGAATGGACGTATGGGGCCAGGG |
| | | AACAACTGTCACCGTCTCCTCA |
| 483 | BIIB-12-1296_VH | EVQLLESGGGLVQPGGSLRLSCAASGFTFSTYAMSWVRQAPGKGLEWVSAISGSGGSTYYADSVKGRFTI |
| | Amino Acid | SRDNSKNTLYLQMNSLRAEDTAVYYCAKGPPHYYYLWYFDLWGRGTLVTVSS |
| | Sequence | |
| 484 | BIIB-12-1296_VH | GAGGTGCAGCTGTTGGAGTCTGGGGGAGGCTTGGTACAGCCTGGGGGGTCCCTGAGACTCTCCTGTGCAG |
| | Nucleic Acid | CCTCTGGATTCACCTTTAGCACCTATGCCATGAGCTGGGTCCGCCAGGCTCCAGGGAAGGGGCTGGAGTG |
| | Sequence | GGTCTCAGCTATTAGTGGTAGTGGTGGTAGCACATACTACGCAGACTCCGTGAAGGGCCGGTTCACCATC |
| | | TCCAGAGACAATTCCAAGAACACGCTGTATCTGCAAATGAACAGCCTGAGAGCCGAGGACACGGCGGTGT |
| | | ACTACTGCGCCAAGGGCCCTCCTCACTACTACTATCTCTGGTACTTCGACCTATGGGGGAGAGGTACCTT |
| | | GGTCACCGTCTCCTCA |
| 485 | BIIB-12-1297_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCARSGGQTHRRSMDVWGQGTTVTVSS |
| | Sequence | |
| 486 | BIIB-12-1297_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAGATCAGGCGGACAAACACACAGGAGGTCAATGGACGTATGGGGCCAGGGAACAACTGT |
| | | CACCGTCTCCTCA |
| 487 | BIIB-12-1298_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCAKGGSDRRVGSWGQGTLVTVSS |
| | Sequence | |
| 488 | BIIB-12-1298_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAAGGGAGGTTCTGACCGCAGAGTGGGCAGTTGGGGACAGGGTACATTGGTCACCGTCTC |
| | | CTCA |
| 489 | BIIB-12-1299_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCARGGSRRAYVYWGQGTLVTVSS |
| | Sequence | |
| 490 | BIIB-12-1299_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAGAGGCGGTTCTAGAAGGGCCTACGTTTATTGGGGACAGGGTACATTGGTCACCGTCTC |
| | | CTCA |
| 491 | BIIB-12-1300_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCARGSKYLHAWGQGTLVTVSS |
| | Sequence | |
| 492 | BIIB-12-1300_VH | AGGTGCAGCTGGTGGAGTCTGGGGGAGGCGTGGTCCAGCCTGGGAGGTCCCTGAGACTCTCCTGTGCAGC |
| | Nucleic Acid | GTCTGGATTCACCTTCAGTAGCTATGGCATGCACTGGGTCCGCCAGGCTCCAGGCAAGGGGCTGGAGTGG |
| | Sequence | GTGGCAGTTATATCGTATGATGGAAGTAATAAATACTATGCAGACTCCGTGAAGGGCCGATTCACCATCT |
| | | CCAGAGACAATTCCAAGAACACGCTGTATCTGCAAATGAACAGCCTGAGAGCCGAGGACACGGCGGTGTA |
| | | CTACTGCGCCAGAGGTTCTAAATACCTCCACGCATGGGGACAGGGTACATTGGTCACCGTCTCCTCA |
| 493 | BIIB-12-1301_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCARTGNYGRGMPYWGQGTLVTVSS |
| | Sequence | |
| 494 | BIIB-12-1301_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAGAACTGGAAACTACGGAAGGGGAATGCCATACTGGGGACAGGGTACATTGGTCACCGT |
| | | CTCCTCA |
| 495 | BIIB-12-1302_VH | QVQLVQSGAEVKKPGASVKVSCKASGYTFTNYGISWVRQAPGQGLEWMGWISAYNGNTNYAQKLQGRVTM |
| | Amino Acid | TTDTSTSTAYMELRSLRSDDTAVYYCARARSDWRAFDIWGQGTMVTVSS |
| | Sequence | |
| 496 | BIIB-12-1302_VH | CAGGTTCAGCTGGTGCAGTCTGGAGCTGAGGTGAAGAAGCCTGGGGCCTCAGTGAAGGTCTCCTGCAAGG |
| | Nucleic Acid | CTTCTGGTTACACCTTTACCAACTATGGTATCAGCTGGGTGCGACAGGCCCCTGGACAAGGGCTTGAGTG |
| | Sequence | GATGGGATGGATCAGCGCTTACAATGGTAACACAAACTATGCACAGAAGCTCCAGGGCAGAGTCACCATG |
| | | ACCACAGACACATCCACGAGCACAGCCTACATGGAGCTGAGGAGCCTGAGATCTGACGACACGGCGGTGT |
| | | ACTACTGCGCAAGGGCTAGAAGTGACTGGAGAGCCTTCGATATATGGGGTCAGGGTACAATGGTCACCGT |
| | | CTCCTCA |
| 497 | BIIB-12-1303_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCARGPTSRLFQHWGQGTLVTVS |
| | Sequence | |
| 498 | BIIB-12-1303_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAGAGGACCCACTAGTAGGTTATTCCAACACTGGGGACAGGGTACATTGGTCACCGTCTC |
| | | CTCA |
| 499 | BIIB-12-1304_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCAKGPGSRRFDIWGQGTMVTVSS |
| | Sequence | |
| 500 | BIIB-12-1304_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAAGGGGCCTGGAAGTAGGAGGTTCGACATATGGGGTCAGGGTACAATGGTCACCGTCTC |
| | | CTCA |
| 501 | BIIB-12-1305_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCAKLGGRWSSDFQHWGQGTLVTVSS |
| | Sequence | |
| 502 | BIIB-12-1305_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAAGCTAGGCGGAAGATGGAGTTCTGACTTCCAACACTGGGGACAGGGTACATTGGTCAC |
| | | CGTCTCCTCA |
| 503 | BIIB-12-1306_VH | QVQLQESGPGLVKPSQTLSLTCTVSGGSISSGGYYWSWIRQHPGKGLEWIGSIYYSGSTYYNPSLKSRVT |
| | Amino Acid | ISVDTSKNQFSLKLSSVTAADTAVYYCARGAGGSAPWGQGTLVTVSS |
| | Sequence | |
| 504 | BIIB-12-1306_VH | CAGGTGCAGCTGCAGGAGTCGGGCCCAGGACTGGTGAAGCCTTCACAGACCCTGTCCCTCACCTGTACTG |
| | Nucleic Acid | TCTCTGGTGGCTCCATCAGCAGTGGTGGTTACTACTGGAGCTGGATCCGCCAGCACCCAGGGAAGGGCCT |
| | Sequence | GGAGTGGATTGGGTCAATCTATTACAGTGGGAGCACCTACTACAACCCGTCCCTCAAGAGTCGAGTTACC |
| | | ATATCAGTAGACACGTCTAAGAACCAGTTCTCCCTGAAGCTGAGTTCTGTGACCGCCGCAGACACGGCGG |
| | | TGTACTACTGCGCCAGAGGTGCCGGAGGATCTGCCCCATGGGGACAGGGTACATTGGTCACCGTCTCCTC |
| | | A |
| 505 | BIIB-12-1307_VH | QVQLQESGPGLVKPSETLSLTCAVSGYSISSGYYWAWIRQPPGKGLEWIGSIYHSGSTYYNPSLKSRVTI |
| | Amino Acid | SVDTSKNQFSLKLSSVTAADTAVYYCARGPLPRSRGLAFDIWGQGTMVTVSS |
| | Sequence | |
| 506 | BIIB-12-1307_VH | CAGGTGCAGCTGCAGGAGTCGGGCCCAGGACTGGTGAAGCCTTCGGAGACCCTGTCCCTCACCTGCGCTG |
| | Nucleic Acid | TCTCTGGTTACTCCATCAGCAGTGGTTACTACTGGGCTTGGATCCGGCAGCCCCCAGGGAAGGGGCTGGA |
| | Sequence | GTGGATTGGGAGTATCTATCATAGTGGGAGCACCTACTACAACCCGTCCCTCAAGAGTCGAGTCACCATA |
| | | TCAGTAGACACGTCCAAGAACCAGTTCTCCCTGAAGCTGAGTTCTGTGACCGCCGCAGACACGGCGGTGT |
| | | ACTACTGCGCCAGAGGTCCCTTGCCAAGATCTAGAGGCTTAGCCTTCGATATCTGGGGTCAGGGTACAAT |
| | | GGTCACCGTCTCCTCA |
| 507 | BIIB-12-1308_VH | QVQLVESGGGVVQPGRSLRLSRAASGFTFSSYGMHWVRQAPGKGLEWVAVISYDGSNKYYADSVKGRFTI |
| | Amino Acid | SRDNSKNTLYLQMNSLRAEDTAVYYCARGPRALGTAFDIWGQGTMVTVSS |
| | Sequence | |
| 508 | BIIB-12-1308_VH | CAGGTGCAGCTGGTGGAGTCTGGGGGAGGCGTGGTCCAGCCTGGGAGGTCCCTGAGACTATCCAGAGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAGAGGACCAAGAGCATTGGGAACCGCATTCGACATATGGGGTCAGGGTACAATGGTCAC |
| | | CGTCTCCTCA |
| 509 | BIIB-12-1309_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCARGRYTSRYFQHWGQGTLVTVSS |
| | Sequence | |
| 510 | BIIB-12-1309_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAGAGGCAGATACACAAGCAGATACTTCCAACACTGGGGACAGGGTACATTGGTCACCGT |
| | | CTCCTCA |
| 511 | BIIB-12-1310_VH | EVQLVESGGGLVKPGGSLRLSCAASGFTFSSYSMNWVRQAPGKGLEWVSSISSSSSYIYYADSVKGRFTI |
| | Amino Acid | SRDNAKNSLYLQMNSLRAEDTAVYYCARLGGYGSSQRYFDLWGRGTLVTVSS |
| | Sequence | |
| 512 | BIIB-12-1310_VH | GAGGTGCAGCTGGTGGAGTCTGGGGGAGGCCTGGTCAAGCCTGGGGGGTCCCTGAGACTCTCCTGTGCAG |
| | Nucleic Acid | CCTCTGGATTCACCTTCAGTAGCTATAGCATGAACTGGGTCCGCCAGGCTCCAGGGAAGGGGCTGGAGTG |
| | Sequence | GGTCTCATCCATTAGTAGTAGTAGTAGTTACATATACTACGCAGACTCAGTGAAGGGCCGATTCACCATC |
| | | TCCAGAGACAACGCCAAGAACTCACTGTATCTGCAAATGAACAGCCTGAGAGCCGAGGACACGGCGGTGT |
| | | ACTACTGCGCCAGATTGGGCGGATACGGAAGCTCGCAGCGATACTTCGACCTATGGGGGAGAGGTACCTT |
| | | GGTCACCGTCTCCTCA |
| 513 | BIIB-12-1311_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCAKGRHYWAVWGQGTLVTVSS |
| | Sequence | |
| 514 | BIIB-12-1311_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAAGGGCAGACACTACTGGGCCGTCTGGGGACAGGGTACATTGGTCACCGTCTCCTCA |
| 515 | BIIB-12-1312_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCAKGMGHWIDYWGQGTLVTVSS |
| | Sequence | |
| 516 | BIIB-12-1312_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAAGGGAATGGGACACTGGATTGACTACTGGGGACAGGGTACATTGGTCACCGTCTCCTC |
| | | A |
| 517 | BIIB-12-1313_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCAKGTGWWRYWGQGTLVTVSS |
| | Sequence | |
| 518 | BIIB-12-1313_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAAGGGAACCGGCTGGTGGCGATACTGGGGACAGGGTACATTGGTCACCGTCTCCTCA |
| 519 | BIIB-12-1314_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCAKGTRWAGNWGQGTLVTVSS |
| | Sequence | |
| 520 | BIIB-12-1314_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAAGGGAACTAGATGGGCAGGGAATTGGGGACAGGGTACATTGGTCACCGTCTCCTCA |
| 521 | BIIB-12-1315_VH | QVQLVQSGAEVKKPGASVKVSCKASGYTFTSYYMVWVRQAPGQGLEWMGIINPSGGSTSYAQKFQGRVTM |
| | Amino Acid | TRDTSTSTVYMELSSLRSEDTAVYYCARGRPSSKRVTYFDYWGQGTLVTVSS |
| | Sequence | |
| 522 | BIIB-12-1315_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGGTCTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGGAAGGCCTAGCAGCAAAAGGGTTACATACTTCGACTACTGGGGACAGGGTACATT |
| | | GGTCACCGTCTCCTCA |
| 523 | BIIB-12-1316_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCAKSGQYRAFDIWGQGTMVTVSS |
| | Sequence | |
| 524 | BIIB-12-1316_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAAGTCTGGACAGTATAGAGCCTTTGATATTTGGGGTCAGGGTACAATGGTCACCGTCTC |
| | | CTCA |
| 525 | BIIB-12-1317_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCARGVGGHDTRWGQGTLVTVSS |
| | Sequence | |
| 526 | BIIB-12-1317_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAGAGGCGTAGGAGGACACGACACGAGATGGGGACAGGGTACATTGGTCACCGTCTCCTC |
| | | A |
| 527 | BIIB-12-1318_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCARKGDYRSGSYSGRAFGIWGQGTMVTVSS |
| | Sequence | |
| 528 | BIIB-12-1318_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAGAAAGGGAGACTACAGGAGCGGAAGCTACTCCGGAAGAGCCTTCGGTATATGGGGTCA |
| | | GGGTACAATGGTCACCGTCTCCTCA |
| 529 | BIIB-12-1319_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCARTGYHRSVYYWGQGTLVTVSS |
| | Sequence | |
| 530 | BIIB-12-1319_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAGAACTGGATACCACAGAAGTGTATACTATTGGGGACAGGGTACATTGGTCACCGTCTC |
| | | CTCA |
| 531 | BIIB-12-1322_VH | QVQLVQSGAEVKKPGASVKVSCKASGYTFTSYYIHWVRQAPGQGLEWMGIINPSGGSTSYAQKFQGRVTM |
| | Amino Acid | TRDTSTSTVYMELSSLRSEDTAVYYCARSRQRHRGDWYFDLWGRGTLVTVSS |
| | Sequence | |
| 532 | BIIB-12-1322_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATCCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCTAGATCAAGACAAAGACACAGAGGTGATTGGTACTTCGATTTATGGGGGAGAGGTACCTT |
| | | GGTCACCGTCTCCTCA |
| 533 | BIIB-12-1323_VH | QVQLVQSGAEVKKPGASVKVSCKASGYTFTSYYIHWVRQAPGQGLEWMGIINPSGGSTSYAQKFQGRVTM |
| | Amino Acid | TRDTSTSTVYMELSSLRSEDTAVYYCARGRRFPRGLDYWGQGTLVTVSS |
| | Sequence | |
| 534 | BIIB-12-1323_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATCCACTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGGCAGAAGATTCCCTAGAGGCTTAGATTACTGGGGACAGGGTACATTGGTCACCGT |
| | | CTCCTCA |
| 535 | BIIB-12-1324_VH | QVQLVQSGAEVKKPGASVKVSCKASGYTFTSYYMVWVRQAPGQGLEWMGIINPSGGSTSYAQKFQGRVTM |
| | Amino Acid | TRDTSTSTVYMELSSLRSEDTAVYYCARLPRYSKRGLDVWGQGTMVTVSS |
| | Sequence | |
| 536 | BIIB-12-1324_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGGTCTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGACTTCCTAGATACAGCAAAAGAGGTCTAGACGTATGGGGTCAGGGTACAATGGTCAC |
| | | CGTCTCCTCA |
| 537 | BIIB-12-1325_VH | QVQLVQSGAEVKKPGASVKVSCKASGYTFTSYYMVWVRQAPGQGLEWMGIINPSGGSTSYAQKFQGRVTM |
| | Amino Acid | TRDTSTSTVYMELSSLRSEDTAVYYCARGGRYMLDPWGQGTLVTVSS |
| | Sequence | |
| 538 | BIIB-12-1325_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGGTCTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGGAGGTAGATATATGTTAGACCCATGGGGACAGGGTACATTGGTCACCGTCTCCTC |
| | | A |
| 539 | BIIB-12-1326_VH | EVQLLESGGGLVQPGGSLRLSCAASGFTFSSYAMSWVRQAPGKGLEWVSTISGSGGSTYYADSVKGRFTI |
| | Amino Acid | SRDNSKNTLYLQMNSLRAEDTAVYYCAKGPLYRRYGYGMDVWGQGTTVTVSS |
| | Sequence | |
| 540 | BIIB-12-1326_VH | GAGGTGCAGCTGTTGGAGTCTGGGGGAGGCTTGGTACAGCCTGGGGGGTCCCTGAGACTCTCCTGTGCAG |
| | Nucleic Acid | CCTCTGGATTCACCTTTAGCAGCTATGCCATGAGCTGGGTCCGCCAGGCTCCAGGGAAGGGGCTGGAGTG |
| | Sequence | GGTCTCAACCATTAGTGGTAGTGGTGGTAGCACATACTACGCAGACTCCGTGAAGGGCCGGTTCACCATC |
| | | TCCAGAGACAATTCCAAGAACACGCTGTATCTGCAAATGAACAGCCTGAGAGCCGAGGACACGGCGGTGT |
| | | ACTACTGCGCCAAGGGGCCGTTGTACCGCAGATACGGCTACGGTATGGACGTTTGGGGCCAGGGAACAAC |
| | | TGTCACCGTCTCCTCA |
| 541 | BIIB-12-1327_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCAKLGLARGGGYGMDVWGQGTTVTVSS |
| | Sequence | |
| 542 | BIIB-12-1327_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAAGTTAGGATTGGCAAGAGGAGGTGGATACGGAATGGACGTATGGGGCCAGGGAACAAC |
| | | TGTCACCGTCTCCTCA |
| 543 | BIIB-12-1328_VH | QLQLQESGPGLVKPSETLSLTCTVSGGSISSSSYYWGWIRQPPGKGLEWIGSIYYSGSTYYNPSLKSRVT |
| | Amino Acid | ISVDTSKNQFSLKLSSVTAADTAVYYCARDPAYYSGHDYYYYGMDVWGQGTTVTVSS |
| | Sequence | |
| 544 | BIIB-12-1328_VH | CAGCTGCAGCTGCAGGAGTCGGGCCCAGGACTGGTGAAGCCTTCGGAGACCCTGTCCCTCACCTGCACTG |
| | Nucleic Acid | TCTCTGGTGGCTCCATCAGCAGTAGTAGTTACTACTGGGGCTGGATCCGCCAGCCCCCAGGGAAGGGGCT |
| | Sequence | GGAGTGGATTGGGAGTATCTATTATAGTGGGAGCACCTACTACAACCCGTCCCTCAAGAGTCGAGTCACC |
| | | ATATCCGTAGACACGTCCAAGAACCAGTTCTCCCTGAAGCTGAGTTCTGTGACCGCCGCAGACACGGCGG |
| | | TGTACTACTGCGCCAGAGATCCCGCTTACTACTCAGGTCACGACTATTACTATTATGGAATGGATGTATG |
| | | GGGCCAGGGAACAACTGTCACCGTCTCCTCA |
| 545 | BIIB-12-1329_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCARGGAGSRYFQHWGQGTLVTVSS |
| | Sequence | |
| 546 | BIIB-12-1329_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAGAGGAGGTGCTGGATCAAGGTACTTCCAACACTGGGGACAGGGTACATTGGTCACCGT |
| | | CTCCTCA |
| 547 | BIIB-12-1330_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCAKGGSARWINIWGQGTTVTVSS |
| | Sequence | |
| 548 | BIIB-12-1330_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAAGGGAGGTTCTGCAAGATGGATCAACATATGGGGTCAGGGAACAACTGTCACCGTCTC |
| | | CTCA |
| 549 | BIIB-12-1331_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCAKVPHHRGHDYWGQGTLVTVSS |
| | Sequence | |
| 550 | BIIB-12-1331_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | ACTACTGCGCCAAGGTACCTCACCACAGAGGTCACGATTACTGGGGACAGGGTACATTGGTCACCGTCTC |
| | | CTCA |
| 551 | BIIB-12-1332 VH | QVQLVQSGAEVKKPGASVKVSCKASGYTFTSYYMSWVRQAPGQGLEWMGIINPSGGSTSYAQKFQGRVTM |
| | Amino Acid | TRDTSTSTVYMELSSLRSEDTAVYYCARLGRKSRVFDIWGQGTMVTVSS |
| | Sequence | |
| 552 | BIIB-12-1332_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGTCATGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGATTGGGAAGGAAATCAAGGGTATTCGACATATGGGGTCAGGGTACAATGGTCACCGT |
| | | CTCCTCA |
| 553 | BIIB-12-1333_VH | QVQLVQSGAEVKKPGASVKVSCKASGYTFTSYYMVWVRQAPGQGLEWMGIINPSGGSTSYAQKFQGRVTM |
| | Amino Acid | TRDTSTSTVYMELSSLRSEDTAVYYCARVSRRDYPLAFDIWGQGTMVTVSS |
| | Sequence | |
| 554 | BIIB-12-1333_VH | CAGGTGCAGCTGGTGCAGTCTGGGGCTGAGGTGAAGAAGCCTGGGGCCTCAGTGAAGGTTTCCTGCAAGG |
| | Nucleic Acid | CATCTGGATACACCTTCACCAGCTACTATATGGTCTGGGTGCGACAGGCCCCTGGACAAGGGCTTGAGTG |
| | Sequence | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCTAGAGTATCTAGAAGGGACTACCCATTAGCCTTCGATATCTGGGGTCAGGGTACAATGGT |
| | | CACCGTCTCCTCA |
| 555 | BIIB-12-1334_VH | QVQLVESGGGVVQPGRSLRLSCAASGFTFSSYGMHWVRQAPGKGLEWVAVISYDGSNKYYADSVKGRFTI |
| | Amino Acid | SRDNSKNTLYLQMNSLRAEDTAVYYCARVPRKQTGHVDYWGQGTLVTVSS |
| | Sequence | |
| 556 | BIIB-12-1334_VH | CAGGTGCAGCTGGTGGAGTCTGGGGGAGGCGTGGTCCAGCCTGGGAGGTCCCTGAGACTCTCCTGTGCAG |
| | Nucleic Acid | CGTCTGGATTCACCTTCAGTAGCTATGGCATGCACTGGGTCCGCCAGGCTCCAGGCAAGGGGCTGGAGTG |
| | Sequence | GGTGGCAGTTATATCGTATGATGGAAGTAATAAATACTATGCAGACTCCGTGAAGGGCCGATTCACCATC |
| | | TCCAGAGACAATTCCAAGAACACGCTGTATCTGCAAATGAACAGCCTGAGAGCCGAGGACACGGCGGTGT |
| | | TACTACTGCGCCAGAGTCCCTAGAAAGCAAACTGGTCACGTGGACTACGGGGACAGGGTACATTGGTCAC |
| | | CGTCTCCTCA |

Class VI Antibodies - VH Sequences

| | | |
|---|---|---|
| 557 | BIIB-12-894_VH | QLQLQESGPGLVKPSETLSLTCTVSGGSISSSSYYWGWIRQPPGKGLEWIGSIYYSGSTYYNPSLKSRVT |
| | Amino Acid | ISVDTSKNQFSLKLSSVTAADTAVYYCAREGAHSSMAGLDVWGQGTMVTVSS |
| | Sequence | |
| 558 | BIIB-12-894_VH | CAGCTGCAGCTGCAGGAGTCGGGCCCAGGACTGGTGAAGCCTTCGGAGACCCTGTCCCTCACCTGCACTG |
| | Nucleic Acid | TCTCTGGTGGCTCCATCAGCAGTAGTAGTTACTACTGGGGCTGGATCCGCCAGCCCCCAGGGAAGGGGCT |
| | Sequence | GGAGTGGATTGGGAGTATCTATTATAGTGGGAGCACCTACTACAACCCGTCCCTCAAGAGTCGAGTCACC |
| | | ATATCCGTAGACACGTCCAAGAACCAGTTCTCCCTGAAGCTGAGTTCTGTGACCGCCGCAGACACGGCGG |
| | | TGTACTACTGCGCCAGAGAAGGAGCTCACAGCAGCATGGCAGGGCTAGACGTATGGGGTCAGGGTACAAT |
| | | GGTCACCGTCTCCTCA |
| 559 | BIIB-12-925_VH | EVQLLESGGGLVQPGGSLRLSCAASGFTFSSYAMSWVRQAPGKGLEWVSAISGSGGSTYYADSVKGRFTI |
| | Amino Acid | SRDNSKNTLYLQMNSLRAEDTAVYYCAKGPRYYWYSWYFDLWGRGTLVTVSS |
| | Sequence | |
| 560 | BIIB-12-925_VH | GAGGTGCAGCTGTTGGAGTCTGGGGGAGGCTTGGTACAGCCTGGGGGGTCCCTGAGACTCTCCTGTGCAG |
| | Nucleic Acid | CCTCTGGATTCACCTTTAGCAGCTATGCCATGAGCTGGGTCCGCCAGGCTCCAGGGAAGGGGCTGGAGTG |
| | Sequence | GGTCTCAGCTATTAGTGGTAGTGGTGGTAGCACATACTACGCAGACTCCGTGAAGGGCCGGTTCACCATC |
| | | TCCAGAGACAATTCCAAGAACACGCTGTATCTGCAAATGAACAGCCTGAGAGCCGAGGACACGGCGGTGT |
| | | ACTACTGCGCCAAGGGCCCCAGATACTACTGGTACAGCTGGTACTTCGACCTATGGGGGAGAGGTACCTT |
| | | GGTCACCGTCTCCTCA |
| 561 | BIIB-12-1320_VH | QLQLQESGPGLVKPSETLSLTCTVSGGSISSSSYYWGWIRQPPGKGLEWIGSIYYSGSTYYNPSLKSRVT |
| | Amino Acid | ISVDTSKNQFSLKLSSVTAADTAVYYCARGSGLLVREHYYYYMDVWGKGTTVTVS |
| | Sequence | |
| 562 | BIIB-12-1320_VH | CAGCTGCAGCTGCAGGAGTCGGGCCCAGGACTGGTGAAGCCTTCGGAGACCCTGTCCCTCACCTGCACTG |
| | Nucleic Acid | TCTCTGGTGGCTCCATCAGCAGTAGTAGTTACTACTGGGGCTGGATCCGCCAGCCCCCAGGGAAGGGGCT |
| | Sequence | GGAGTGGATTGGGAGTATCTATTATAGTGGGAGCACCTACTACAACCCGTCCCTCAAGAGTCGAGTCACC |
| | | ATATCCGTAGACACGTCCAAGAACCAGTTCTCCCTGAAGCTGAGTTCTGTGACCGCCGCAGACACGGCGG |
| | | TGTACTACTGCGCCAGAGGCTCTGGATTGCTAGTCCGAGAGCACTACTACTACTACATGGACGTATGGGG |
| | | CAAGGGTACAACTGTCACCGTCTCCTCA |
| 563 | BIIB-12-1321_VH | QVQLVQSGAEVKKPGSSVKVSCKASGGTFSSYAISWVRQAPGQGLEWMGGIIPIFGTANYAQKFQGRVTI |
| | Amino Acid | TADESTSTAYMELSSLRSEDTAVYYCARTPDTSSATDWGQGTLVTVSS |
| | Sequence | |
| 564 | BIIB-12-1321_VH | CAGGTGCAGCTGGTGCAGTCTGGGGCTGAGGTGAAGAAGCCTGGGTCCTCGGTGAAGGTCTCCTGCAAGG |
| | Nucleic Acid | CTTCTGGAGGCACCTTCAGCAGCTATGCTATCAGCTGGGTGCGACAGGCCCCTGGACAAGGGCTTGAGTG |
| | Sequence | GATGGGAGGGATCATCCCTATCTTTGGTACAGCAAACTACGCACAGAAGTTCCAGGGCAGAGTCACGATT |
| | | ACCGCGGACGAATCCACGAGCACAGCCTACATGGAGCTGAGCAGCCTGAGATCTGAGGACACGGCGGTGT |
| | | ACTACTGCGCCAGAACTCCTGACACAAGCTCTGCTACCGATTGGGGACAGGGTACATTGGTCACCGTCTC |
| | | CTCA |

Class V Antibodies - VL Sequences

| | | |
|---|---|---|
| 565 | BIIB-12-891_VL | DIVMTQSPDSLAVSLGERATINCKSSQSVLYSSNNKNYLAWYQQKPGQPPKLLIYWASTRESGVPDRFSG |
| | Amino Acid | SGSGTDFTLTISSLQAEDVAVYYCQQYFNPPFTFGGGTKVEIK |
| | Sequence | |
| 566 | BIIB-12-891_VL | GACATCGTGATGACCCAGTCTCCAGACTCCCTGGCTGTGTCTCTGGGCGAGAGGGCCACCATCAACTGCA |
| | Nucleic Acid | AGTCCAGCCAGAGTGTTTTATACAGCTCCAACAATAAGAACTACTTAGCTTGGTACCAGCAGAAACCAGG |
| | Sequence | ACAGCCTCCTAAGCTGCTCATTTACTGGGCATCTACCCGGGAATCCGGGGTCCCTGACCGATTCAGTGGC |
| | | AGCGGGTCTGGGACAGATTTCACTCTCACCATCAGCAGCCTGCAGGCTGAAGATGTGGCAGTTTATTACT |
| | | GTCAGCAGTACTTCAACCCCCCTTTCACTTTTGGCGGAGGGACCAAGGTTGAGATCAAA |
| 567 | BIIB-12-892_VL | EIVLTQSPATLSLSPGERATLSCRASQSVSSYLAWYQQKPGQAPRLLIYDASNRATGIPARFSGSGSGTD |
| | Amino Acid | FTLTISSLEPEDFAVYYCQQRLNLPLTFGGGTKVEIK |
| | Sequence | |
| 568 | BIIB-12-892_VL | GAAATTGTGTTGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATTTATGATGCATCCAACAGGGCCACTGGCATCCCAGCCAGGTTCAGTGGCAGTGGGTCTGGGACAGAT |
| | | TTCACTCTCACCATCAGCAGCCTAGAGCCTGAAGATTTTGCAGTTTATTACTGTCAGCAGAGACTCAACC |
| | | TCCCTCTCACTTTTGGCGGAGGGACCAAGGTTGAGATCAAA |
| 569 | BIIB-12-893_VL | DIQLTQSPSSVSASVGDRVTITCRASQGISSWLAWYQQKPGKAPKLLIYAASSLQSGVPSRFSGSGSGTD |
| | Amino Acid | FTLTISSLQPEDFATYYCQQAAAFPFTFGGGTKVEIK |
| | Sequence | |
| 570 | BIIB-12-893_VL | GACATCCAGTTGACCCAGTCTCCATCTTCCGTGTCTGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGCAGCCGCCT |
| | | TCCCTTTCACTTTTGGCGGAGGGACCAAGGTTGAGATCAAA |
| 571 | BIIB-12-895_VL | DIQMTQSPSSVSASVGDRVTITCRASQGISSWLAWYQQKPGKAPKLLIYAASSLQSGVPSRFSGSGSGTD |
| | Amino Acid | FTLTISSLQPEDFATYYCQQASSFPFTFGGGTKVEI |
| | Sequence | |
| 572 | BIIB-12-895_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGCATCCAGTT |
| | | TCCCTTTCACTTTTGGCGGAGGGACCAAGGTTGAGATCAAA |
| 573 | BIIB-12-896_VL | DIQMTQSPSSVSASVGDRVTITCRASQGISSWLAWYQQKPGKAPKLLIYAASSLQSGVPSRFSGSGSGTD |
| | Amino Acid | FTLTISSLQPEDFATYYCQQANSFPFTFGGGTKVEIK |
| | Sequence | |
| 574 | BIIB-12-896_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGCAAATTCCT |
| | | TCCCTTTCACTTTTGGCGGAGGGACCAAGGTTGAGATCAAA |
| 575 | BIIB-12-897_VL | EIVLTQSPGTLSLSPGERATLSCRASQSVSSSYLAWYQQKPGQAPRLLIYGASSRATGIPDRFSGSGSGT |
| | Amino Acid | DFTLTISRLEPEDFAVYYCQQDGNYPYTFGGGTKVEIK |
| | Sequence | |
| 576 | BIIB-12-897_VL | GAAATTGTGTTGACGCAGTCTCCAGGCACCCTGTCTTTGTCTCCAGGGGAAAGAGCCACCCTCTCCTGCA |
| | Nucleic Acid | GGGCCAGTCAGAGTGTTAGCAGCAGCTACTTAGCCTGGTACCAGCAGAAACCTGGCCAGGCTCCCAGGCT |
| | Sequence | CCTCATCTATGGTGCATCCAGCAGGGCCACTGGCATCCCAGACAGGTTCAGTGGCAGTGGGTCTGGGACA |
| | | GACTTCACTCTCACCATCAGCAGACTGGAGCCTGAAGATTTTGCAGTGTATTACTGTCAGCAGGACGGAA |
| | | ACTACCCTTACACTTTTGGCGGAGGGACCAAGGTTGAGATCAAA |
| 577 | BIIB-12-898_VL | DIQLTQSPSTLSASVGDRVTITCRASQAISSWLAWYQQKPGKAPKLLIYDASSLESGVPSRFSGSGSGTE |
| | Amino Acid | FTLTISSLQPDDFATYYCQQVNRFPFTFGGGTKVEIK |
| | Sequence | |
| 578 | BIIB-12-898_VL | GACATCCAGTTGACCCAGTCTCCTTCCACCCTGTCTGCATCTGTAGGAGACAGAGTCACCATCACTTGCC |
| | Nucleic Acid | GGGCCAGTCAGGCTATTAGTAGCTGGTTGGCCTGGTATCAGCAGAAACCAGGGAAAGCCCCTAAGCTCCT |
| | Sequence | GATCTATGATGCCTCCAGTTTGGAAAGTGGGGTCCCATCAAGGTTCAGCGGCAGTGGATCTGGGACAGAA |
| | | TTCACTCTCACCATCAGCAGCCTGCAGCCTGATGATTTTGCAACTTATTACTGCCAGCAGGTCAATCGCT |
| | | TCCCTTTCACTTTTGGCGGAGGGACCAAGGTTGAGATCAAA |
| 579 | BIIB-12-899_VL | DIVMTQSPDSLAVSLGERATINCKSSQSVLYSSNNKNYLAWYQQKPGQPPKLLIYWASTRESGVPDRFSG |
| | Amino Acid | SGSGTDFTLTISSLQAEDVAVYYCQQSYTLPPTFGGGTKVEIK |
| | Sequence | |
| 580 | BIIB-12-899_VL | GACATCGTGATGACCCAGTCTCCAGACTCCCTGGCTGTGTCTCTGGGCGAGAGGGCCACCATCAACTGCA |
| | Nucleic Acid | AGTCCAGCCAGAGTGTTTTATACAGCTCCAACAATAAGAACTACTTAGCTTGGTACCAGCAGAAACCAGG |
| | Sequence | ACAGCCTCCTAAGCTGCTCATTTACTGGGCATCTACCCGGGAATCCGGGGTCCCTGACCGATTCAGTGGC |
| | | AGCGGGTCTGGGACAGATTTCACTCTCACCATCAGCAGCCTGCAGGCTGAAGATGTGGCAGTTTATTACT |
| | | GTCAGCAGTCCTACACCCTCCCTCCTACTTTTGGCGGAGGGACCAAGGTTGAGATCAAA |
| 581 | BIIB-12-900_VL | DIQMTQSPSTLSASVGDRVTITCRASQSISSWLAWYQQKPGKAPKLLIYKASSLESGVPSRFSGSGSGTE |
| | Amino Acid | FTLTISSLQPDDFATYYCQQYRSYPTFGGGTKVEIK |
| | Sequence | |
| 582 | BIIB-12-900_VL | GACATCCAGATGACCCAGTCTCCTTCCACCCTGTCTGCATCTGTAGGAGACAGAGTCACCATCACTTGCC |
| | Nucleic Acid | GGGCCAGTCAGAGTATTAGTAGCTGGTTGGCCTGGTATCAGCAGAAACCAGGGAAAGCCCCTAAGCTCCT |
| | Sequence | GATCTATAAAGCCTCCAGTTTGGAAAGTGGGGTCCCATCAAGGTTCAGCGGCAGTGGATCTGGGACAGAA |
| | | TTCACTCTCACCATCAGCAGCCTGCAGCCTGATGATTTTGCAACTTATTACTGCCAGCAGTACCGAAGCT |
| | | ACCCTACTTTTGGCGGAGGGACCAAGGTTGAGATCAAA |
| 583 | BIIB-12-901_VL | EIVLTQSPATLSLSPGERATLSCRASQSVSSYLAWYQQKPGQAPRLLIYDASNRATGIPARFSGSGSGTD |
| | Amino Acid | FTLTISSLEPEDFAVYYCQQSANFPFTFGGGTKVEIK |
| | Sequence | |
| 584 | BIIB-12-901_VL | GAAATTGTGTTGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGTCCGCCAATT |
| | | TCCCTTTCACTTTTGGCGGAGGGACCAAGGTTGAGATCAAA |
| 585 | BIIB-12-902_VL | DIVMTQSPDSLAVSLGERATINCKSSQSVLYSSNNKNYLAWYQQKPGQPPKLLISWASTRESGVPDRFSG |
| | Amino Acid | SGSGTDFTLTISSLQAEDVAVYYCQQSFSTPFTFGGGTKVEIK |
| | Sequence | |
| 586 | BIIB-12-902_VL | GACATCGTGATGACCCAGTCTCCAGACTCCCTGGCTGTGTCTCTGGGCGAGAGGGCCACCATCAACTGCA |
| | Nucleic Acid | AGTCCAGCCAGAGTGTTTTATACAGCTCCAACAATAAGAACTACTTAGCTTGGTACCAGCAGAAACCAGG |
| | Sequence | ACAGCCTCCTAAGCTGCTCATTTCCTGGGCATCTACCCGGGAATCCGGGGTCCCTGACCGATTCAGTGGC |
| | | AGCGGGTCTGGGACAGATTTCACTCTCACCATCAGCAGCCTGCAGGCTGAAGATGTGGCAGTTTATTACT |
| | | GTCAGCAGTCCTTCTCCACTCCTTTCACTTTTGGCGGAGGGACCAAGGTTGAGATCAAA |
| 587 | BIIB-12-903_VL | EIVLTQSPATLSLSPGERATLSCRASQSVSSYLAWYQQKPGQAPRLLIYDASNRATGIPARFSGSGSGTD |
| | Amino Acid | FTLTISSLEPEDFAVYYCQQRANWPPTFGGGTKVEIK |
| | Sequence | |
| 588 | BIIB-12-903_VL | GAAATTGTGTTGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGAGAGCCAACT |
| | | GGCCTCCTACTTTTGGCGGAGGGACCAAGGTTGAGATCAAA |
| 589 | BIIB-12-904_VL | EIVMTQSPATLSVSPGERATLSCRASQSVSSNLAWYQQKPGQAPRLLIYGASTRATGIPARFSGSGSGTE |
| | Amino Acid | FTLTISSLQSEDFAVYYCQQAFNWPPTFGGGTKVEIK |
| | Sequence | |
| 590 | BIIB-12-904_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGGCCTTCAACT |
| | | GGCCTCCTACTTTTGGCGGAGGGACCAAGGTTGAGATCAAA |
| 591 | BIIB-12-905_VL | EIVLTQSPGTLSLSPGERATLSCRASQSVRSSYLAWYQQKPGQAPRLLIYGASSRATGIPDRFSGSGSGT |
| | Amino Acid | DFTLTISRLEPEDFAVYYCQQYGRFPPTFGGGTKVEIK |
| | Sequence | |
| 592 | BIIB-12-905_VL | GAAATTGTGTTGACGCAGTCTCCAGGCACCCTGTCTTTGTCTCCAGGGGAAAGAGCCACCCTCTCCTGCA |
| | Nucleic Acid | GGGCCAGTCAGAGTGTTAGGAGCAGCTACTTAGCCTGGTACCAGCAGAAACCTGGCCAGGCTCCCAGGCT |
| | Sequence | CCTCATCTATGGTGCATCCAGCAGGGCCACTGGCATCCCAGACAGGTTCAGTGGCAGTGGGTCTGGGACA |
| | | GACTTCACTCTCACCATCAGCAGACTGGAGCCTGAAGATTTTGCAGTGTATTACTGTCAGCAGTACGGAC |
| | | GCTTCCCTCCTACTTTTGGCGGAGGGACCAAGGTTGAGATCAAA |
| 593 | BIIB-12-906_VL | EIVLTQSPATLSLSPGERATLSCRASQSVSSYLAWYQQKPGQAPRLLIYDASNRATGIPARFSGSGSGTD |
| | Amino Acid | FTLTISSLEPEDFAVYYCQQSSDWPPTFGGGTKVEIK |
| | Sequence | |
| 594 | BIIB-12-906_VL | GAAATTGTGTTGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGTCCTCCGACT |
| | | GGCCTCCTACTTTTGGCGGAGGGACCAAGGTTGAGATCAAA |
| 595 | BIIB-12-907_VL | DIVMTQSPLSLPVTPGEPASISCRSSQSLLYSNGYNYLDWYLQKPGQSPQLLIYLGSNRASGVPDRFSGS |
| | Amino Acid | GSGTDFTLKISRVEAEDVGVYYCMQRLGLPPTFGGGTKVEIK |
| | Sequence | |
| 596 | BIIB-12-907_VL | GATATTGTGATGACTCAGTCTCCACTCTCCCTGCCCGTCACCCCTGGAGAGCCGGCCTCCATCTCCTGCA |
| | Nucleic Acid | GGTCTAGTCAGAGCCTCCTGTATAGTAATGGATACAACTATTTGGATTGGTACCTGCAGAAGCCAGGGCA |
| | Sequence | GTCTCCACAGCTCCTGATCTATTTGGGTTCTAATCGGGCCTCCGGGGTCCCTGACAGGTTCAGTGGCAGT |
| | | GGATCAGGCACAGATTTTACACTGAAAATCAGCAGAGTGGAGGCTGAGGATGTTGGGGTTTATTACTGCA |
| | | TGCAGAGACTCGGCCTCCCTCCTACTTTTGGCGGAGGGACCAAGGTTGAGATCAAA |
| 597 | BIIB-12-908_VL | DIQLTQSPSSVSASVGDRVTITCRASQGISSWLAWYQQKPGKAPKLLIYGASSLQSGVPSRFSGSGSGTD |
| | Amino Acid | FTLTISSLQPEDFATYYCQQGSSLPITFGGGTKVEIK |
| | Sequence | |
| 598 | BIIB-12-908_VL | GACATCCAGTTGACCCAGTCTCCATCTTCCGTGTCTGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGGTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGGATCCAGTC |
| | | TCCCTATCACTTTTGGCGGAGGGACCAAGGTTGAGATCAAA |
| 599 | BIIB-12-909_VL | EIVLTQSPATLSLSPGERATLSCRASQSVSSYLAWYQQKPGQAPRLLIYDASNRATGIPARFSGSGSGTD |
| | Amino Acid | FTLTISSLEPEDFAVYYCQQLSDWPPTFGGGTKVEIK |
| | Sequence | |
| 600 | BIIB-12-909_VL | GAAATTGTGTTGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGCTCAGTGACT |
| | | GGCCTCCTACTTTTGGCGGAGGGACCAAGGTTGAGATCAAA |
| 601 | BIIB-12-910_VL | DIQMTQSPSSLSASVGDRVTITCRASQSISSYLNWYQQKPGKAPKLLIYAASSLQSGVPSRFSGSGSGTD |
| | Amino Acid | FTLTISSLQPEDFATYYCQQLSHTPFTFGGGTKVEIK |
| | Sequence | |
| 602 | BIIB-12-910_VL | GACATCCAGATGACCCAGTCTCCATCCTCCCTGTCTGCATCTGTAGGAGACAGAGTCACCATCACTTGCC |
| | Nucleic Acid | GGGCAAGTCAGAGCATTAGCAGCTATTTAAATTGGTATCAGCAGAAACCAGGGAAAGCCCCTAAGCTCCT |
| | Sequence | GATCTATGCTGCATCCAGTTTGCAAAGTGGGGTCCCATCAAGGTTCAGTGGCAGTGGATCTGGGACAGAT |
| | | TTCACTCTCACCATCAGCAGTCTGCAACCTGAAGATTTTGCAACTTACTACTGTCAGCAACTATCCCACA |
| | | CTCCTTTCACTTTTGGCGGAGGGACCAAGGTTGAGATCAAA |
| 603 | BIIB-12-911_VL | EIVLTQSPATLSLSPGERATLSCRASQSVSSYLAWYQQKPGQAPRLLIYDASNRATGIPARFSGSGSGTD |
| | Amino Acid | FTLTISSLEPEDFAVYYCQQRSNFPITFGGGTKVEIK |
| | Sequence | |
| 604 | BIIB-12-911_VL | GAAATTGTGTTGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGAGAAGTAACT |
| | | TCCCTATCACTTTTGGCGGAGGGACCAAGGTTGAGATCAAA |
| 605 | BIIB-12-912_VL | EIVMTQSPATLSVSPGERATLSCRASQSVSSNLAWYQQKPGQAPRLLIYGASTRATGIPARFSGSGSGTE |
| | Amino Acid | FTLTISSLQSEDFAVYYCQQFHNFPPTFGGGTKVEIK |
| | Sequence | |
| 606 | BIIB-12-912_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGTTCCACAATT |
| | | TCCCTCCTACTTTTGGCGGAGGGACCAAGGTTGAGATCAA |
| 607 | BIIB-12-913_VL | EIVMTQSPATLSVSPGERATLSCRASQSVSSNLAWYQQKPGQAPRLLIYGASTRATGIPARFSGSGSGTE |
| | Amino Acid | FTLTISSLQSEDFAVYYCQQYAPYPPLTFGGGTKVEIK |
| | Sequence | |
| 608 | BIIB-12-913_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGTACGCCCCCT |
| | | ACCCTCCTCTCACTTTTGGCGGAGGGACCAAGGTTGAGATCAAA |
| 609 | BIB-12-914_VL | DIQMTQSPSSLSASVGDRVTITCRASQSISSYLNWYQQKPGKAPKLLIYAASSLQSGVPSRFSGSGSGTD |
| | Amino Acid | FTLTISSLQPEDFATYYCQQGYNTPLTFGGGTKVEIK |
| | Sequence | |
| 610 | BIIB-12-914_VL | GACATCCAGATGACCCAGTCTCCATCCTCCCTGTCTGCATCTGTAGGAGACAGAGTCACCATCACTTGCC |
| | Nucleic Acid | GGGCAAGTCAGAGCATTAGCAGCTATTTAAATTGGTATCAGCAGAAACCAGGGAAAGCCCCTAAGCTCCT |
| | Sequence | GATCTATGCTGCATCCAGTTTGCAAAGTGGGGTCCCATCAAGGTTCAGTGGCAGTGGATCTGGGACAGAT |
| | | TTCACTCTCACCATCAGCAGTCTGCAACCTGAAGATTTTGCAACTTACTACTGTCAGCAAGGATACAACA |
| | | CTCCTCTCACTTTTGGCGGAGGGACCAAGGTTGAGATCAAA |
| 611 | BIIB-12-915_VL | DIVMTQSPLSLPVTPGEPASISCRSSQSLLHSNGYNYLDWYLQKPGQSPQLLIYLASNRASGVPDRFSGS |
| | Amino Acid | GSGTDFTLKISRVEAEDVGVYYCMQARQRPWTFGGGTKVEIK |
| | Sequence | |
| 612 | BIIB-12-915_VL | GATATTGTGATGACTCAGTCTCCACTCTCCCTGCCCGTCACCCCTGGAGAGCCGGCCTCCATCTCCTGCA |
| | Nucleic Acid | GGTCTAGTCAGAGCCTCCTGCATAGTAATGGATACAACTATTTGGATTGGTACCTGCAGAAGCCAGGGCA |
| | Sequence | GTCTCCACAGCTCCTGATCTATTTGGCTTCTAATCGGGCCTCCGGGGTCCCTGACAGGTTCAGTGGCAGT |
| | | GGATCAGGCACAGATTTTACACTGAAAATCAGCAGAGTGGAGGCTGAGGATGTTGGGGTTTATTACTGCA |
| | | TGCAGGCAAGACAGCGCCCTTGGACTTTTGGCGGAGGGACCAAGGTTGAGATCAAA |
| 613 | BIIB-12-916_VL | DIQMTQSPSSLSASVGDRVTITCRASQSISSYLNWYQQKPGKAPKLLIYAASSLQSGVPSRFSGSGSGTD |
| | Amino Acid | FTLTISSLQPEDFATYYCQQLAITPFTFGGGTKVEIK |
| | Sequence | |
| 614 | BIIB-12-916_VL | GACATCCAGATGACCCAGTCTCCATCCTCCCTGTCTGCATCTGTAGGAGACAGAGTCACCATCACTTGCC |
| | Nucleic Acid | GGGCAAGTCAGAGCATTAGCAGCTATTTAAATTGGTATCAGCAGAAACCAGGGAAAGCCCCTAAGCTCCT |
| | Sequence | GATCTATGCTGCATCCAGTTTGCAAAGTGGGGTCCCATCAAGGTTCAGTGGCAGTGGATCTGGGACAGAT |
| | | TTCACTCTCACCATCAGCAGTCTGCAACCTGAAGATTTTGCAACTTACTACTGTCAGCAACTAGCCATCA |
| | | CTCCTTTCACTTTTGGCGGAGGGACCAAGGTTGAGATCAAA |
| 615 | BIIB-12-917_VL | EIVMTQSPATLSVSPGERATLSCRASQSVSSNLAWYQQKPGQAPRLLIYGASTRATGIPARFSGSGSGTE |
| | Amino Acid | FTLTISSLQSEDFAVYYCQQLFNHPPTFGGGTKVEIK |
| | Sequence | |
| 616 | BIIB-12-917_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGCTCTTCAATC |
| | | ACCCTCCTACTTTTGGCGGAGGGACCAAGGTTGAGATCAAA |
| 617 | BIIB-12-918_VL | DIQMTQSPSSLSASVGDRVTITCRASQSISRYLNWYQQKPGKAPKLLIYAASSLQSGVPSRFSGSGSGTD |
| | Amino Acid | FTLTISSLQPEDFATYYCQQIFSLPITFGGGTKVEIK |
| | Sequence | |
| 618 | BIIB-12-918_VL | GACATCCAGATGACCCAGTCTCCATCCTCCCTGTCTGCATCTGTAGGAGACAGAGTCACCATCACTTGCC |
| | Nucleic Acid | GGGCAAGTCAGAGCATTAGCAGATATTTAAATTGGTATCAGCAGAAACCAGGGAAAGCCCCTAAGCTCCT |
| | Sequence | GATCTATGCTGCATCCAGTTTGCAAAGTGGGGTCCCATCAAGGTTCAGTGGCAGTGGATCTGGGACAGAT |
| | | TTCACTCTCACCATCAGCAGTCTGCAACCTGAAGATTTTGCAACTTACTACTGTCAGCAAATATTCAGTC |
| | | TCCCTATCACTTTTGGCGGAGGGACCAAGGTTGAGATCAAA |
| 619 | BIIB-12-919_VL | DIQMTQSPSTLSASVGDRVTITCRASQSISSWLAWYQQKPGKAPKLLIYKASSLESGVPSRFSGSGSGTE |
| | Amino Acid | FTLTISSLQPDDFATYYCQQVSSYPTFGGGTKVEIK |
| | Sequence | |
| 620 | BIIB-12-919_VL | GACATCCAGATGACCCAGTCTCCTTCCACCCTGTCTGCATCTGTAGGAGACAGAGTCACCATCACTTGCC |
| | Nucleic Acid | GGGCCAGTCAGAGTATTAGTAGCTGGTTGGCCTGGTATCAGCAGAAACCAGGGAAAGCCCCTAAGCTCCT |
| | Sequence | GATCTATAAAGCCTCCAGTTTGGAAAGTGGGGTCCCATCAAGGTTCAGCGGCAGTGGATCTGGGACAGAA |
| | | TTCACTCTCACCATCAGCAGCCTGCAGCCTGATGATTTTGCAACTTATTACTGCCAGCAGGTCAGCAGTT |
| | | ACCCTACTTTTGGCGGAGGGACCAAGGTTGAGATCAAA |
| 621 | BIIB-12-920_VL | EIVMTQSPATLSVSPGERATLSCRASQSVSSNLAWYQQKPGQAPRLLIYSASTRATGIPARFSGSGSGTE |
| | Amino Acid | FTLTISSLQSEDFAVYYCQQANAYPPTFGGGTKVEI |
| | Sequence | |
| 622 | BIIB-12-920_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATAGCGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGGCCAATGCCT |
| | | ACCCTCCTACTTTTGGCGGAGGGACCAAGGTTGAGATCAAA |
| 623 | BIIB-12-921_VL | DIQMTQSPSSVSASVGDRVTITCRASQGISRWLAWYQQKPGKAPKLLIYAASSLQSGVPSRFSGSGSGTD |
| | Amino Acid | FTLTISSLQPEDFATYYCQQGNVFPLTFGGGTKVEIK |
| | Sequence | |
| 624 | BIIB-12-921_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCAGGTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTACTACTGTCAGCAGGGAAATGTCT |
| | | TCCCTCTCACTTTTGGCGGAGGGACCAAGGTTGAGATCAAA |
| 625 | BIIB-12-922_VL | DIVMTQSPLSLPVTPGEPASISCRSSQSLLHSNGYNYLDWYLQKPGQSPQLLIYLGSNRASGVPDRFSGS |
| | Amino Acid | GSGTDFTLKISRVEAEDVGVYYCMQALQTPITFGGGTKVEIK |
| | Sequence | |
| 626 | BIIB-12-922_VL | GATATTGTGATGACTCAGTCTCCACTCTCCCTGCCCGTCACCCCTGGAGAGCCGGCCTCCATCTCCTGCA |
| | Nucleic Acid | GGTCTAGTCAGAGCCTCCTGCATAGTAATGGATACAACTATTTGGATTGGTACCTGCAGAAGCCAGGGCA |
| | Sequence | GTCTCCACAGCTCCTGATCTATTTGGGTTCTAATCGGGCCTCCGGGGTCCCTGACAGGTTCAGTGGCAGT |
| | | GGATCAGGCACAGATTTTACACTGAAAATCAGCAGAGTGGAGGCTGAGGATGTTGGGGTTTATTACTGCA |
| | | TGCAGGCACTACAGACTCCTATCACTTTTGGCGGAGGGACCAAGGTTGAGATCAAA |
| 627 | BIIB-12-923_VL | DIQMTQSPSTLSASVGDRVTITCRASQSISSWLAWYQQKPGKAPKLLIYKASSLESGVPSRFSGSGSGTE |
| | Amino Acid | FTLTISSLQPDDFATYYCQQFKSLSFTFGGGTKVEIK |
| | Sequence | |
| 628 | BIIB-12-923_VL | GACATCCAGATGACCCAGTCTCCTTCCACCCTGTCTGCATCTGTAGGAGACAGAGTCACCATCACTTGCC |
| | Nucleic Acid | GGGCCAGTCAGAGTATTAGTAGCTGGTTGGCCTGGTATCAGCAGAAACCAGGGAAAGCCCCTAAGCTCCT |
| | Sequence | GATCTATAAAGCCTCCAGTTTGGAAAGTGGGGTCCCATCAAGGTTCAGCGGCAGTGGATCTGGGACAGAA |
| | | TTCACTCTCACCATCAGCAGCCTGCAGCCTGATGATTTTGCAACTTATTACTGCCAGCAGTTCAAAAGTC |
| | | TCTCTTTCACTTTTGGCGGAGGGACCAAGGTTGAGATCAAA |
| 629 | BIIB-12-924_VL | DIQMTQSPSSVSASVGDRVTITCRASQGISSWLAWYQQKPGKAPKLLIYAASSLQSGVPSRFSGSGSGTD |
| | Amino Acid | FTLTISSLQPEDFATYYCQQGNSFPLTFGGGTKVEIK |
| | Sequence | |
| 630 | BIIB-12-924_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGGAAATTCCT |
| | | TCCCTCTCACTTTTGGCGGAGGGACCAAGGTTGAGATCAAA |
| 631 | BIIB-12-926_VL | EIVMTQSPATLSVSPGERATLSCRASQSVSSNLAWYQQKPGQAPRLLIYGASTRATGIPARFSGSGSGTE |
| | Amino Acid | FTLTISSLQSEDFAVYYCQQHSFWPPTFGGGTKVEIK |
| | Sequence | |
| 632 | BIIB-12-926_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGCACTCCTTCT |
| | | GGCCTCCTACTTTTGGCGGAGGGACCAAGGTTGAGATCAAA |
| 633 | BIIB-12-927_VL | EIVMTQSPATLSVSPGERATLSCRASQSVSSNLAWYQQKPGQAPRLLIYGASTRATGIPARFSGSGSGTE |
| | Amino Acid | FTLTISSLQSEDFAVYYCQQFNLWPFTFGGGTKVEIK |
| | Sequence | |
| 634 | BIIB-12-927_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGTTCAATCTCT |
| | | GGCCTTTCACTTTTGGCGGAGGGACCAAGGTTGAGATCAAA |
| 635 | BIIB-12-928_VL | EIVLTQSPGTLSLSPGERATLSCRASQSVRSSYLAWYQQKPGQAPRLLIYGASSRATGIPDRFSGSGSGT |
| | Amino Acid | DFTLTISRLEPEDFAVYYCQQYVRFPLTFGGGTKVEIK |
| | Sequence | |
| 636 | BIIB-12-928_VL | GAAATTGTGTTGACGCAGTCTCCAGGCACCCTGTCTTTGTCTCCAGGGGAAAGAGCCACCCTCTCCTGCA |
| | Nucleic Acid | GGGCCAGTCAGAGTGTTAGGAGCAGCTACTTAGCCTGGTACCAGCAGAAACCTGGCCAGGCTCCCAGGCT |
| | Sequence | CCTCATCTATGGTGCATCCAGCAGGGCCACTGGCATCCCAGACAGGTTCAGTGGCAGTGGGTCTGGGACA |
| | | GACTTCACTCTCACCATCAGCAGACTGGAGCCTGAAGATTTTGCAGTGTATTACTGTCAGCAGTACGTAC |
| | | GTTTCCCTCTCACTTTTGGCGGAGGGACCAAGGTTGAGATCAAA |
| 637 | BIIB-12-929_VL | DIVMTQSPDSLAVSLGERATINCKSSQSVLYSSNNKNYLAWYQQKPGQPPKLLISWASTRESGVPDRFSG |
| | Amino Acid | SGSGTDFTLTISSLQAEDVAVYYCQQYFSLPFTFGGGTKVEIK |
| | Sequence | |
| 638 | BIIB-12-929_VL | GACATCGTGATGACCCAGTCTCCAGACTCCCTGGCTGTGTCTCTGGGCGAGAGGGCCACCATCAACTGCA |
| | Nucleic Acid | AGTCCAGCCAGAGTGTTTTATACAGCTCCAACAATAAGAACTACTTAGCTTGGTACCAGCAGAAACCAGG |
| | Sequence | ACAGCCTCCTAAGCTGCTCATTTCCTGGGCATCTACCCGGGAATCCGGGGTCCCTGACCGATTCAGTGGC |
| | | AGCGGGTCTGGGACAGATTTCACTCTCACCATCAGCAGCCTGCAGGCTGAAGATGTGGCAGTTTATTACT |
| | | GTCAGCAGTACTTCAGTCTCCCTTTCACTTTTGGCGGAGGGACCAAGGTTGAGATCAAA |
| 639 | BIIB-12-930_VL | DIVMTQSPDSLAVSLGERATINCKSSQSVLFSSNNKNYLAWYQQKPGQPPKLLIYWASTRESGVPDRFSG |
| | Amino Acid | SGSGTDFTLTISSLQAEDVAVYYCQQYFLSPFTFGGGTKVEIK |
| | Sequence | |
| 640 | BIIB-12-930_VL | GACATCGTGATGACCCAGTCTCCAGACTCCCTGGCTGTGTCTCTGGGCGAGAGGGCCACCATCAACTGCA |
| | Nucleic Acid | AGTCCAGCCAGAGTGTTTTATTCAGCTCCAACAATAAGAACTACTTAGCTTGGTACCAGCAGAAACCAGG |
| | Sequence | ACAGCCTCCTAAGCTGCTCATTTACTGGGCATCTACCCGGGAATCCGGGGTCCCTGACCGATTCAGTGGC |
| | | AGCGGGTCTGGGACAGATTTCACTCTCACCATCAGCAGCCTGCAGGCTGAAGATGTGGCAGTTTATTACT |
| | | GTCAGCAGTACTTCCTCTCCCCTTTCACTTTTGGCGGAGGGACCAAGGTTGAGATCAAA |
| 641 | BIIB-12-931_VL | EIVMTQSPATLSVSPGERATLSCRASQSVSSNLAWYQQKPGQAPRLLIYGASTRATGIPARFSGSGSGTE |
| | Amino Acid | FTLTISSLQSEDFAVYYCQQAAYWPWTFGGGTKVEIK |
| | Sequence | |
| 642 | BIIB-12-931_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGGCCGCCTACT |
| | | GGCCTTGGACTTTTGGCGGAGGGACCAAGGTTGAGATCAAA |
| 643 | BIIB-12-932_VL | DIQMTQSPSSLSASVGDRVTITCRASQSISSYLNWYQQKPGKAPKLLIYAASSLQSGVPSRFSGSGSGTD |
| | Amino Acid | FTLTISSLQPEDFATYYCQQSHSAPTFGGGTKVEIK |
| | Sequence | |
| 644 | BIIB-12-932_VL | GACATCCAGATGACCCAGTCTCCATCCTCCCTGTCTGCATCTGTAGGAGACAGAGTCACCATCACTTGCC |
| | Amino Acid | GGGCAAGTCAGAGCATTAGCAGCTATTTAAATTGGTATCAGCAGAAACCAGGGAAAGCCCCTAAGCTCCT |
| | Sequence | GATCTATGCTGCATCCAGTTTGCAAAGTGGGGTCCCATCAAGGTTCAGTGGCAGTGGATCTGGGACAGAT |
| | | TTCACTCTCACCATCAGCAGTCTGCAACCTGAAGATTTTGCAACTTACTACTGTCAGCAAAGCCACAGTG |
| | | CCCCTACTTTTGGCGGAGGGACCAAGGTTGAGATCAAA |
| 645 | BIIB-12-933_VL | EIVLTQSPGTLSLSPGERATLSCRASQSVSSSYLAWYQQKPGQAPRLLIYGASSRATGIPDRFSGSGSGT |
| | Amino Acid | DFTLTISRLEPEDFAVYYCQQYSRSPITFGGGTKVEIK |
| | Sequence | |
| 646 | BIIB-12-933_VL | GAAATTGTGTTGACGCAGTCTCCAGGCACCCTGTCTTTGTCTCCAGGGGAAAGAGCCACCCTCTCCTGCA |
| | Nucleic Acid | GGGCCAGTCAGAGTGTTAGCAGCAGCTACTTAGCCTGGTACCAGCAGAAACCTGGCCAGGCTCCCAGGCT |
| | Sequence | CCTCATCTATGGTGCATCCAGCAGGGCCACTGGCATCCCAGACAGGTTCAGTGGCAGTGGGTCTGGGACA |
| | | GACTTCACTCTCACCATCAGCAGACTGGAGCCTGAAGATTTTGCAGTGTATTACTGTCAGCAGTACTCCC |
| | | GCTCCCCTATCACTTTTGGCGGAGGGACCAAGGTTGAGATCAAA |
| 647 | BIIB-12-934_VL | EIVLTQSPATLSVSPGERATLSCRASQSVSSNLAWYQQKPGQAPRLLIYGASTRATGIPARFSGSGSGTE |
| | Amino Acid | FTLTISSLQSEDFAVYYCQQFNDHPITFGGGTKVEI |
| | Sequence | |
| 648 | BIIB-12-934_VL | GAAATAGTGTTGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGTTCAATGACC |
| | | ACCCTATCACTTTTGGCGGAGGGACCAAGGTTGAGATCAAA |
| 649 | BIIB-12-935_VL | EIVLTQSPGTLSLSPGERATLSCRASQSVSSSFLAWYQQKPGQAPRLLIYGASSRATGIPDRFSGSGSGT |
| | Amino Acid | DFTLTISRLEPEDFAVYYCQQYSSSPITFGGGTKVEIK |
| | Sequence | |
| 650 | BIIB-12-935_VL | GAAATTGTGTTGACGCAGTCTCCAGGCACCCTGTCTTTGTCTCCAGGGGAAAGAGCCACCCTCTCCTGCA |
| | Nucleic Acid | GGGCCAGTCAGAGTGTTAGCAGCAGCTTCTTAGCCTGGTACCAGCAGAAACCTGGCCAGGCTCCCAGGCT |
| | Sequence | CCTCATCTATGGTGCATCCAGCAGGGCCACTGGCATCCCAGACAGGTTCAGTGGCAGTGGGTCTGGGACA |
| | | GACTTCACTCTCACCATCAGCAGACTGGAGCCTGAAGATTTTGCAGTGTATTACTGTCAGCAGTACTCCA |
| | | GTTCCCCTATCACTTTTGGCGGAGGGACCAAGGTTGAGATCAAA |
| 651 | BIIB-12-936_VL | EIVMTQSPATLSVSPGERATLSCRASQSVSSNLAWYQQKPGQAPRLLIYGASTRATGIPARFSGSGSGTE |
| | Amino Acid | FTLTISSLQSEDFAVYYCQQSHNLPPTFGGGTKVEIK |
| | Sequence | |
| 652 | BIIB-12-936_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGTCCCACAATC |
| | | TCCCTCCTACTTTTGGCGGAGGGACCAAGGTTGAGATCAAA |
| 653 | BIIB-12-937_VL | DIVMTQSPLSLPVTPGEPASISCRSSQSLLHSNGYNYLDWYLQKPGQSPQLLIYLGSNRASGVPDRFSGS |
| | Amino Acid | GSGTDFTLKISRVEAEDVGVYYCMQERQTPLTFGGGTKVEIK |
| | Sequence | |
| 654 | BIIB-12-937_VL | GATATTGTGATGACTCAGTCTCCACTCTCCCTGCCCGTCACCCCTGGAGAGCCGGCCTCCATCTCCTGCA |
| | Nucleic Acid | GGTCTAGTCAGAGCCTCCTGCATAGTAATGGATACAACTATTTGGATTGGTACCTGCAGAAGCCAGGGCA |
| | Sequence | GTCTCCACAGCTCCTGATCTATTTGGGTTCTAATCGGGCCTCCGGGGTCCCTGACAGGTTCAGTGGCAGT |
| | | GGATCAGGCACAGATTTTACACTGAAAATCAGCAGAGTGGAGGCTGAGGATGTTGGGGTTTATTACTGCA |
| | | TGCAGGAAAGACAAACTCCTCTCACTTTTGGCGGAGGGACCAAGGTTGAGATCAAA |
| 655 | BIIB-12-1288_VL | DIVMTQSPDSLAVSLGERATINCKSSQSVLYSSNNKNYLAWYQQKPGQPPKLLIYWASTRESGVPDRFSG |
| | Amino Acid | SGSGTDFTLTISSLQAEDVAVYYCQQSFNTPFTFGGGTKVEIK |
| | Sequence | |
| 656 | BIIB-12-1288_VL | GACATCGTGATGACCCAGTCTCCAGACTCCCTGGCTGTGTCTCTGGGCGAGAGGGCCACCATCAACTGCA |
| | Nucleic Acid | AGTCCAGCCAGAGTGTTTTATACAGCTCCAACAATAAGAACTACTTAGCTTGGTACCAGCAGAAACCAGG |
| | Sequence | ACAGCCTCCTAAGCTGCTCATTTACTGGGCATCTACCCGGGAATCCGGGGTCCCTGACCGATTCAGTGGC |
| | | AGCGGGTCTGGGACAGATTTCACTCTCACCATCAGCAGCCTGCAGGCTGAAGATGTGGCAGTTTATTACT |
| | | GTCAGCAGTCCTTCAACACTCCTTTCACTTTTGGCGGAGGGACCAAGGTTGAGATCAAA |
| 657 | BIIB-12-1289_VL | DIVMTQSPDSLAVSLGERATINCKSSQSVLYSSNNKNYLAWYQQKPGQPPKLLIYWASTRESGVPDRFSG |
| | Amino Acid | SGSGTDFTLTISSLQAEDVAVYYCQQYYASPFTFGGGTKVEIK |
| | Sequence | |
| 658 | BIIB-12-1289_VL | GACATCGTGATGACCCAGTCTCCAGACTCCCTGGCTGTGTCTCTGGGCGAGAGGGCCACCATCAACTGCA |
| | Nucleic Acid | AGTCCAGCCAGAGTGTTTTATACAGCTCCAACAATAAGAACTACTTAGCTTGGTACCAGCAGAAACCAGG |
| | Sequence | ACAGCCTCCTAAGCTGCTCATTTACTGGGCATCTACCCGGGAATCCGGGGTCCCTGACCGATTCAGTGGC |
| | | AGCGGGTCTGGGACAGATTTCACTCTCACCATCAGCAGCCTGCAGGCTGAAGATGTGGCAGTTTATTACT |
| | | GTCAGCAGTACTACGCCTCCCCTTTCACTTTTGGCGGAGGGACCAAGGTTGAGATCAAA |
| 659 | BIIB-12-1290_VL | DIQMTQSPSSVSASVGDRVTITCRASQGISSWLAWYQQKPGKAPKLLIYAASSLQSGVPSRFSGSGSGTD |
| | Amino Acid | FTLTISSLQPEDFATYYCQQGFSFPFTFGGGTKVEIK |
| | Sequence | |
| 660 | BIIB-12-1290_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGGATTCAGTT |
| | | TCCCTTTCACTTTTGGCGGAGGGACCAAGGTTGAGATCAAA |
| 661 | BIIB-12-1291_VL | EIVLTQSPATLSLSPGERATLSCRASQSVSSYLAWYQQKPGQAPRLLIYDASNRATGIPARFSGSGSGTD |
| | Amino Acid | FTLTISSLEPEDFAVYYCQQRLNLPLTFGGGTKVEIK |
| | Sequence | |
| 662 | BIIB-12-1291_VL | GAAATTGTGTTGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGAGACTCAATC |
| | | TCCCTCTCACTTTTGGCGGAGGGACCAAGGTTGAGATCAA |
| 663 | BIIB-12-1292_VL | EIVMTQSPATLSLSPGERATLSCRASQSVSSYLAWYQQKPGQAPRLLIYDSSNRATGIPARFSGSGSGTD |
| | Amino Acid | FTLTISSLEPEDFAVYYCQQRLNFPFTFGGGTKVEIK |
| | Sequence | |
| 664 | BIIB-12-1292_VL | GAAATTGTGATGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATTCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGAGACTCAATT |
| | | TCCCTTTCACTTTTGGCGGAGGGACCAAGGTTGAGATCAAA |
| 665 | BIIB-12-1293_VL | DIQMTQSPSSVSASVGDRVTITCRASQGISSWLAWYQQKPGKAPKLLIYAASSLQSGVPSRFSGSGSGTD |
| | Amino Acid | FTLTISSLQPEDFATYYCQQANIFPFTFGGGTKVEIK |
| | Sequence | |
| 666 | BIIB-12-1293_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGCAAATATCT |
| | | TCCCTTTCACTTTTGGCGGAGGGACCAAGGTTGAGATCAAA |
| 667 | BIIB-12-1294_VL | DIQMTQSPSTLSASVGDRVTITCRASQSISSWLAWYQQKPGKAPKLLIYKASSLESGVPSRFSGSGSGTE |
| | Amino Acid | FTLTISSLQPDDFATYYCQQVNTFPFTFGGGTKVEIK |
| | Sequence | |
| 668 | BIIB-12-1294_VL | GACATCCAGATGACCCAGTCTCCTTCCACCCTGTCTGCATCTGTAGGAGACAGAGTCACCATCACTTGCC |
| | Nucleic Acid | GGGCCAGTCAGAGTATTAGTAGCTGGTTGGCCTGGTATCAGCAGAAACCAGGGAAAGCCCCTAAGCTCCT |
| | Sequence | GATCTATAAAGCCTCCAGTTTGGAAAGTGGGGTCCCATCAAGGTTCAGCGGCAGTGGATCTGGGACAGAA |
| | | TTCACTCTCACCATCAGCAGCCTGCAGCCTGATGATTTTGCAACTTATTACTGCCAGCAGGTCAATACCT |
| | | TCCCTTTCACTTTTGGCGGAGGGACCAAGGTTGAGATCAAA |
| 669 | BIIB-12-1295_VL | DIQMTQSPSSVSASVGDRVTITCRASQGISSWLAWYQQKPGKAPKLLIYAASNLQSGVPSRFSGSGSGTD |
| | Amino Acid | FTLTISSLQPEDFATYYCQQAISLPITFGGGTKVEIK |
| | Sequence | |
| 670 | BIIB-12-1295_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAATTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGCAATCAGTC |
| | | TCCCTATCACTTTTGGCGGAGGGACCAAGGTTGAGATCAAA |
| 671 | BIIB-12-1296_VL | EIVLTQSPGTLSLSPGERATLSCRASQSVSSSYLAWYQQKPGQAPRLLIYGASSRATGIPDRFSGSGSGT |
| | Amino Acid | DFTLTISRLEPEDFAVYYCQQYADSPLTFGGGTKVEIK |
| | Sequence | |
| 672 | BIIB-12-1296_VL | GAAATTGTGTTGACGCAGTCTCCAGGCACCCTGTCTTTGTCTCCAGGGGAAAGAGCCACCCTCTCCTGCA |
| | Nucleic Acid | GGGCCAGTCAGAGTGTTAGCAGCAGCTACTTAGCCTGGTACCAGCAGAAACCTGGCCAGGCTCCCAGGCT |
| | Sequence | CCTCATCTATGGTGCATCCAGCAGGGCCACTGGCATCCCAGACAGGTTCAGTGGCAGTGGGTCTGGGACA |
| | | GACTTCACTCTCACCATCAGCAGACTGGAGCCTGAAGATTTTGCAGTGTATTACTGTCAGCAGTACGCCG |
| | | ACAGTCCTCTCACTTTTGGCGGAGGGACCAAGGTTGAGATCAAA |
| 673 | BIIB-12-1297_VL | EIVLTQSPATLSLSPGERATLSCRASQSVSRYLAWYQQKPGQAPRLLIYDASNRATGIPARFSGSGSGTD |
| | Amino Acid | FTLTISSLEPEDFAVYYCQQGNNWPPTFGGGTKVEIK |
| | Sequence | |
| 674 | BIIB-12-1297_VL | GAAATTGTGTTGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGGTACTTAGCCTGGTACCAACAGAAACCTGGCCAGGCTCCCAGGCTCCT |
| | Sequence | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGGGCAACAATT |
| | | GGCCTCCTACTTTTGGCGGAGGGACCAAGGTTGAGATCAAA |
| 675 | BIIB-12-1298_VL | DIQMTQSPSSVSASVGDRVTITCRASQGISSWLAWYQQKPGKAPKLLIYAASSLQSGVPSRFSGSGSGTD |
| | Amino Acid | FTLTISSLQPEDFATYYCQQTNSLPITFGGGTKVEIK |
| | Sequence | |
| 676 | BIIB-12-1298_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGACAAATAGTC |
| | | TCCCTATCACTTTTGGCGGAGGGACCAAGGTTGAGATCAAA |
| 677 | BIIB-12-1299_VL | EIVMTQSPATLSVSPGERATLSCRASQSVSSNLAWYQQKPGQAPRLLIYGASTRATGIPARFSGSGSGTE |
| | Amino Acid | FTLTISSLQSEDFAVYYCQQLNNFPITFGGGTKVEIK |
| | Sequence | |
| 678 | BIIB-12-1299_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGCTCAATAATT |
| | | TCCCTATCACTTTTGGCGGAGGGACCAAGGTTGAGATCAAA |
| 679 | BIIB-12-1300_VL | DIQMTQSPSSVSASVGDRVTITCRASQGISSWLAWYQQKPGKAPKLLIYAASSLQSGVPSRFSGSGSGTD |
| | Amino Acid | FTLTISSLQPEDFATYYCQQASSFPPTFGGGTKVEIK |
| | Sequence | |
| 680 | BIIB-12-1300_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGCATCCAGTT |
| | | TCCCTCCTACTTTTGGCGGAGGGACCAAGGTTGAGATCAAA |
| 681 | BIIB-12-1301_VL | EIVLTQSPATLSLSPGERATLSCRASQSVSSYLAWYQQKPGQAPRLLIYDASKRATGIPARFSGSGSGTD |
| | Amino Acid | FTLTISSLEPEDFAVYYCQQSSSWPPTFGGGTKVEIK |
| | Sequence | |
| 682 | BIIB-12-1301_VL | GAAATTGTGTTGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATGCATCCAAAAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGTCCAGTTCCT |
| | | GGCCTCCTACTTTTGGCGGAGGGACCAAGGTTGAGATCAAA |
| 683 | BIIB-12-1302_VL | EIVMTQSPATLSVSPGERATLSCRASQSVSSNLAWYQQKPGQAPRLLIYGASTRATGIPARFSGSGSGTE |
| | Amino Acid | FTLTISSLQSEDFAVYYCQQVNNLPLTFGGGTKVEIK |
| | Sequence | |
| 684 | BIIB-12-1302_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGGTCAATAATC |
| | | TCCCTCTCACTTTTGGCGGAGGGACCAAGGTTGAGATCAAA |
| 685 | BIIB-12-1303_VL | DIQMTQSPSSLSASVGDRVTITCRASQSISSYLNWYQQKPGKAPKLLIYAASSLQSGVPSRFSGSGSGTD |
| | Amino Acid | FTLTISSLQPEDFATYYCQQAYSLPITFGGGTKVEIK |
| | Sequence | |
| 686 | BIIB-12-1303_VL | GACATCCAGATGACCCAGTCTCCATCCTCCCTGTCTGCATCTGTAGGAGACAGAGTCACCATCACTTGCC |
| | Nucleic Acid | GGGCAAGTCAGAGCATTAGCAGCTATTTAAATTGGTATCAGCAGAAACCAGGGAAAGCCCCTAAGCTCCT |
| | Sequence | GATCTATGCTGCATCCAGTTTGCAAAGTGGGGTCCCATCAAGGTTCAGTGGCAGTGGATCTGGGACAGAT |
| | | TTCACTCTCACCATCAGCAGTCTGCAACCTGAAGATTTTGCAACTTACTACTGTCAGCAAGCATACAGTC |
| | | TCCCTATCACTTTTGGCGGAGGGACCAAGGTTGAGATCAAA |
| 687 | BIIB-12-1304_VL | EIVMTQSPATLSVSPGERATLSCRASQSVSSNLAWYQQKPGQAPRLLIYGASTRATGIPARFSGSGSGTE |
| | Amino Acid | FTLTISSLQSEDFAVYYCQQANVLPLTFGGGTKVEIK |
| | Sequence | |
| 688 | BIIB-12-1304_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGGCCAATGTCC |
| | | TTCCTCTCACTTTTGGCGGAGGGACCAAGGTTGAGATCAAA |
| 689 | BIIB-12-1305_VL | EIVLTQSPATLSLSPGERATLSCRASQSVSSYLAWYQQKPGQAPRLLIYDASNRATGIPARFSGSGSGTD |
| | Amino Acid | FTLTISSLEPEDFAVYYCQQSANWPPTFGGGTKVEIK |
| | Sequence | |
| 690 | BIIB-12-1305_VL | GAAATTGTGTTGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGTCCGCCAATT |
| | | GGCCTCCTACTTTTGGCGGAGGGACCAAGGTTGAGATCAAA |
| 691 | BIIB-12-1306_VL | DIVMTQSPDSLAVSLGERATINCKSSQSVLYSSNNKNYLAWYQQKPGQPPKLLIYWASTRESGVPDRFSG |
| | Amino Acid | SGSGTDFTLTISSLQAEDVAVYYCQQSVNTPLTFGGGTKVEIK |
| | Sequence | |
| 692 | BIIB-12-1306_VL | GACATCGTGATGACCCAGTCTCCAGACTCCCTGGCTGTGTCTCTGGGCGAGAGGGCCACCATCAACTGCA |
| | Nucleic Acid | AGTCCAGCCAGAGTGTTTTATACAGCTCCAACAATAAGAACTACTTAGCTTGGTACCAGCAGAAACCAGG |
| | Sequence | ACAGCCTCCTAAGCTGCTCATTTACTGGGCATCTACCCGGGAATCCGGGGTCCCTGACCGATTCAGTGGC |
| | | AGCGGGTCTGGGACAGATTTCACTCTCACCATCAGCAGCCTGCAGGCTGAAGATGTGGCAGTTTATTACT |
| | | GTCAGCAGTCCGTCAACACTCCTCTCACTTTTGGCGGAGGGACCAAGGTTGAGATCAAA |
| 693 | BIIB-12-1307_VL | DIQLTQSPSSLSASVGDRVTITCRASQSISSFLNWYQQKPGKAPKLLIYAASSLQSGVPSRFSGSGSGTD |
| | Amino Acid | FTLTISSLQPEDFATYYCQQTYSTPLTFGGGTKVEIK |
| | Sequence | |
| 694 | BIIB-12-1307_VL | GACATCCAGTTGACCCAGTCTCCATCCTCCCTGTCTGCATCTGTAGGAGACAGAGTCACCATCACTTGCC |
| | Nucleic Acid | GGGCAAGTCAGAGCATTAGCAGCTTTTTAAATTGGTATCAGCAGAAACCAGGGAAAGCCCCTAAGCTCCT |
| | Sequence | GATCTATGCTGCATCCAGTTTGCAAAGTGGGGTCCCATCAAGGTTCAGTGGCAGTGGATCTGGGACAGAT |
| | | TTCACTCTCACCATCAGCAGTCTGCAACCTGAAGATTTTGCAACTTACTACTGTCAGCAAACATACAGTA |
| | | CTCCTCTCACTTTTGGCGGAGGGACCAAGGTTGAGATCAAA |
| 695 | BIIB-12-1308_VL | EIVLTQSPGTLSLSPGERATLSCRASQSVSSDYLAWYQQKPGQAPRLLIYGASSRATGIPDRFSGSGSGT |
| | Amino Acid | DFTLTISRLEPEDFAVYYCQQAGSFPPTFGGGTKVEIK |
| | Sequence | |
| 696 | BIIB-12-1308_VL | GAAATTGTGTTGACGCAGTCTCCAGGCACCCTGTCTTTGTCTCCAGGGGAAAGAGCCACCCTCTCCTGCA |
| | Nucleic Acid | GGGCCAGTCAGAGTGTTAGCAGCGACTACTTAGCCTGGTACCAGCAGAAACCTGGCCAGGCTCCCAGGCT |
| | Sequence | CCTCATCTATGGTGCATCCAGCAGGGCCACTGGCATCCCAGACAGGTTCAGTGGCAGTGGGTCTGGGACA |
| | | GACTTCACTCTCACCATCAGCAGACTGGAGCCTGAAGATTTTGCAGTGTATTACTGTCAGCAGGCCGGAA |
| | | GTTTCCCTCCTACTTTTGGCGGAGGGACCAAGGTTGAGATCAAA |
| 697 | BIIB-12-1309_VL | DIQMTQSPSSVSASVGDRVTITCRASQGISSWLAWYQQKPGKAPKLLIYAASSLQSGVPSRFSGSGSGTD |
| | Amino Acid | FTLTISSLQPEDFATYYCQQANSLPITFGGGTKVEIK |
| | Sequence | |
| 698 | BIIB-12-1309_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGCAAATTCCC |
| | | TCCCTATCACTTTTGGCGGAGGGACCAAGGTTGAGATCAAA |
| 699 | BIIB-12-1310_VL | EIVLTQSPATLSLSPGERATLSCRASQSVSRYLAWYQQKPGQAPRLLIYDASNRATGIPARFSGSGSGTD |
| | Amino Acid | FTLTISSLEPEDFAVYYCEQASNWPPTFGGGTKVEIK |
| | Sequence | |
| 700 | BIIB-12-1310_VL | GAAATTGTGTTGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGGTACTTAGCCTGGTACCAACAGAAACCTGGCCAGGCTCCCAGGCTCCT |
| | Sequence | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTGAACAGGCCAGTAATT |
| | | GGCCTCCTACTTTTGGCGGAGGGACCAAGGTTGAGATCAAA |
| 701 | BIIB-12-1311_VL | EIVLTQSPATLSLSPGERATLSCRASQSVSSYLAWYQQKPGQAPRLLIYDASNRATGIPARFSGSGSGTD |
| | Amino Acid | FTLTISSLEPEDFAVYYCQQASNLPPTFGGGTKVEIK |
| | Sequence | |
| 702 | BIIB-12-1311_VL | GAAATTGTGTTGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGGCCTCCAATC |
| | | TCCCTCCTACTTTTGGCGGAGGGACCAAGGTTGAGATCAA |
| 703 | BIIB-12-1312_VL | DIQMTQSPSSVSASVGDRVTITCRASQGISSWLAWYQQKPGKAPKLLIYAASSLQSGVPSRFSGSGSGTD |
| | Amino Acid | FTLTISSLQPEDFATYYCQQASSFPPTFGGGTKVEIK |
| | Sequence | |
| 704 | BIIB-12-1312_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGCATCCAGTT |
| | | TCCCTCCTACTTTTGGCGGAGGGACCAAGGTTGAGATCAAA |
| 705 | BIIB-12-1313_VL | EIVMTQSPATLSVSPGERATLSCRASQSVSSNLAWYQQKPGQAPRLLIYGASTRATGIPARFSGSGSGTE |
| | Amino Acid | FTLTISSLQSEDFAVYYCQQHNHLPITFGGGTKVEIK |
| | Sequence | |
| 706 | BIIB-12-1313_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGCACAATCACC |
| | | TCCCTATCACTTTTGGCGGAGGGACCAAGGTTGAGATCAAA |
| 707 | BIIB-12-1314_VL | DIQMTQSPSSVSASVGDRVTITCRASQGISSWLAWYQQKPGKAPKLLIYAASSLQSGVPSRFSGSGSGTD |
| | Amino Acid | FTLTISSLQPEDFATYYCQQANFFPITFGGGTKVEIK |
| | Sequence | |
| 708 | BIIB-12-1314_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGCAAATTTCT |
| | | TCCCTATCACTTTTGGCGGAGGGACCAAGGTTGAGATCAAA |
| 709 | BIIB-12-1315_VL | EIVMTQSPATLSVSPGERATLSCRASQSVSSNLAWYQQKPGQAPRLLIYGASTRATGIPARFSGSGSGTE |
| | Amino Acid | FTLTISSLQSEDFAVYYCQQVFNWPPWTFGGGTKVEIK |
| | Sequence | |
| 710 | BIIB-12-1315_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGGTCTTCAATT |
| | | GGCCTCCTTGGACTTTTGGCGGAGGGACCAAGGTTGAGATCAAA |
| 711 | BIIB-12-1316_VL | DIQMTQSPSSVSASVGDRVTITCRASQGISSWLAWYQQKPGKAPKLLIYAASSLQSGVPSRFSGSGSGTD |
| | Amino Acid | FTLTISSLQPEDFATYYCQQINNFPPTFGGGTKVEIK |
| | Sequence | |
| 712 | BIIB-12-1316_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGATAAATAACT |
| | | TCCCTCCTACTTTTGGCGGAGGGACCAAGGTTGAGATCAAA |
| 713 | BIIB-12-1317_VL | DIQMTQSPSSVSASVGDRVTITCRASQGISSWLAWYQQKPGKAPKLLIYAASSLQSGVPSRFSGSGSGTD |
| | Amino Acid | FTLTISSLQPEDFATYYCQQRHSLPPTFGGGTKVEIK |
| | Sequence | |
| 714 | BIIB-12-1317_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGAGACACAGTC |
| | | TCCCTCCTACTTTTGGCGGAGGGACCAAGGTTGAGATCAAA |
| 715 | BIIB-12-1318_VL | EIVLTQSPATLSLSPGERATLSCRASQSVSSYLAWYQQKPGQAPRLLIYDASNRATGIPARFSGSGSGTD |
| | Amino Acid | FTLTISSLEPEDFAVYYCQQLNNWPPTFGGGTKVEIK |
| | Sequence | |
| 716 | BIIB-12-1318_VL | GAAATTGTGTTGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGCTCAACAATT |
| | | GGCCTCCTACTTTTGGCGGAGGGACCAAGGTTGAGATCAAA |
| 717 | BIIB-12-1319_VL | EIVMTQSPATLSVSPGERATLSCRASQSVSSNLAWYQQKPGQAPRLLIYGASTRATGIPARFSGSGSGTE |
| | Amino Acid | FTLTISSLQSEDFAVYYCQQASNFPPTFGGGTKVEI |
| | Sequence | |
| 718 | BIIB-12-1319_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGGCCTCCAATT |
| | | TCCCTCCTACTTTTGGCGGAGGGACCAAGGTTGAGATCAAA |
| 719 | BIIB-12-1322_VL | DIVMTQSPDSLAVSLGERATINCKSSQSVLYSSNNKNYLAWYQQKPGQPPKLLISWASTRESGVPDRFSG |
| | Amino Acid | SGSGTDFTLTISSLQAEDVAVYYCQQSYFTPFTFGGGTKVEIK |
| | Sequence | |
| 720 | BIIB-12-1322_VL | ACATCGTGATGACCCAGTCTCCAGACTCCCTGGCTGTGTCTCTGGGCGAGAGGGCCACCATCAACTGCAA |
| | Nucleic Acid | GTCCAGCCAGAGTGTTTTATACAGCTCCAACAATAAGAACTACTTAGCTTGGTACCAGCAGAAACCAGGA |
| | Sequence | CAGCCTCCTAAGCTGCTCATTTCCTGGGCATCTACCCGGGAATCCGGGGTCCCTGACCGATTCAGTGGCA |
| | | GCGGGTCTGGGACAGATTTCACTCTCACCATCAGCAGCCTGCAGGCTGAAGATGTGGCAGTTTATTACTG |
| | | TCAGCAGTCCTACTTCACTCCTTTCACTTTTGGCGGAGGGACCAAGGTTGAGATCAAA |
| 721 | BIIB-12-1323_VL | DIQMTQSPSSVSASVGDRVTITCRASQGISSWLAWYQQKPGKAPKLLIYAASSLQSGVPSRFSGSGSGTD |
| | Amino Acid | FTLTISSLQPEDFATYYCQQANIFPFTFGGGTKVEIK |
| | Sequence | |
| 722 | BIIB-12-1323_VL | GACATCCAGATGACCCAGTCTCCATCTTCCGTGTCTGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGCAAATATCT |
| | | TCCCTTTCACTTTTGGCGGAGGGACCAAGGTTGAGATCAAA |
| 723 | BIIB-12-1324_VL | EIVMTQSPATLSLSPGERATLSCRASQSVSSYLAWYQQKPGQAPRLLIYDSSNRATGIPARFSGSGSGTD |
| | Amino Acid | FTLTISSLEPEDFAVYYCQQLVSWPTFGGGTKVEIK |
| | Sequence | |
| 724 | BIIB-12-1324_VL | GAAATTGTGATGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATTCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGCTCGTCTCCT |
| | | GGCCTACTTTTGGCGGAGGGACCAAGGTTGAGATCAAA |
| 725 | BIIB-12-1325_VL | DIQMTQSPSTLSASVGDRVTITCRASQSISSWLAWYQQKPGKAPKLLIYKASSLESGVPSRFSGSGSGTE |
| | Amino Acid | FTLTISSLQPDDFATYYCQQSNRYPRTFGGGTKVEIK |
| | Sequence | |
| 726 | BIIB-12-1325_VL | GACATCCAGATGACCCAGTCTCCTTCCACCCTGTCTGCATCTGTAGGAGACAGAGTCACCATCACTTGCC |
| | Nucleic Acid | GGGCCAGTCAGAGTATTAGTAGCTGGTTGGCCTGGTATCAGCAGAAACCAGGGAAAGCCCCTAAGCTCCT |
| | Sequence | GATCTATAAAGCCTCCAGTTTGGAAAGTGGGGTCCCATCAAGGTTCAGCGGCAGTGGATCTGGGACAGAA |
| | | TTCACTCTCACCATCAGCAGCCTGCAGCCTGATGATTTTGCAACTTATTACTGCCAGCAGTCCAATCGCT |
| | | ACCCTAGGACTTTTGGCGGAGGGACCAAGGTTGAGATCAAA |
| 727 | BIIB-12-1326_VL | EIVLTQSPGTLSLSPGERATLSCRASQSVRSSYLAWYQQKPGQAPRLLIYGASSRATGIPDRFSGSGSGT |
| | Amino Acid | DFTLTISRLEPEDFAVYYCQQAYSSPLTFGGGTKVEIK |
| | Sequence | |
| 728 | BIIB-12-1326_VL | GAAATTGTGTTGACGCAGTCTCCAGGCACCCTGTCTTTGTCTCCAGGGGAAAGAGCCACCCTCTCCTGCA |
| | Nucleic Acid | GGGCCAGTCAGAGTGTTAGGAGCAGCTACTTAGCCTGGTACCAGCAGAAACCTGGCCAGGCTCCCAGGCT |
| | Sequence | CCTCATCTATGGTGCATCCAGCAGGGCCACTGGCATCCCAGACAGGTTCAGTGGCAGTGGGTCTGGGACA |
| | | GACTTCACTCTCACCATCAGCAGACTGGAGCCTGAAGATTTTGCAGTGTATTACTGTCAGCAGGCCTACA |
| | | GTTCCCCTTTGACTTTTGGCGGAGGGACCAAGGTTGAGATCAAA |
| 729 | BIIB-12-1327_VL | EIVMTQSPATQYVSPGERATLSCRASQSVSSNLAWYQQKPGQAPRLLIYGASTRATGIPARFSGSGSGTE |
| | Amino Acid | FTLTISSLQSEDFAVYYCQQYHNFPPTFGGGTKVEIK |
| | Sequence | |
| 730 | BIIB-12-1327_VL | GAAATAGTGATGACGCAGTCTCCAGCCACCCAGTATGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence737 | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGTACCACAATT |
| | | TCCCTCCTACTTTTGGCGGAGGGACCAAGGTTGAGATCAAA |
| 731 | BIIB-12-1328_VL | EIVMTQSPATLSVSPGERATLSCRASQSVSSNLAWYQQKPGQAPRLLIYGASTRATGIPARFSGSGSGTE |
| | Amino Acid | FTLTISSLQSEDFAVYYCQQPNSYPLTFGGGTKVEIK |
| | Sequence | |
| 732 | BIIB-12-1328_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGCCCAATTCCT |
| | | ACCCTCTCACTTTTGGCGGAGGGACCAAGGTTGAGATCAAA |
| 733 | BIIB-12-1329_VL | EIVMTQSPATKSVSPGERATLSCRASQSVSSNLAWYQQKPGQAPRLLIYSASTRATGIPARFSGSGSGTE |
| | Amino Acid | FTLTISSLQSEDFAVYYCQQVFNWPPTFGGGTKVKIK |
| | Sequence | |
| 734 | BIIB-12-1329_VL | GAAATAGTGATGACGCAGTCTCCAGCCACCAAGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATAGCGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGGTCTTCAATT |
| | | GGCCTCCTACTTTTGGCGGAGGGACCAAGGTTAAGATCAAA |
| 735 | BIIB-12-1330_VL | DIQMTQSPSSLSASVGDRVTITCRASQSISSYLNWYQQKPGKAPKLLIYAASSLQSGVPSRFSGSGSGTD |
| | Amino Acid | FTLTISSLQPEDFATYYCQQAYSVPITFGGGTKVEI |
| | Sequence | |
| 736 | BIIB-12-1330_VL | GACATCCAGATGACCCAGTCTCCATCCTCCCTGTCTGCATCTGTAGGAGACAGAGTCACCATCACTTGCC |
| | Nucleic Acid | GGGCAAGTCAGAGCATTAGCAGCTATTTAAATTGGTATCAGCAGAAACCAGGGAAAGCCCCTAAGCTCCT |
| | Sequence | GATCTATGCTGCATCCAGTTTGCAAAGTGGGGTCCCATCAAGGTTCAGTGGCAGTGGATCTGGGACAGAT |
| | | TTCACTCTCACCATCAGCAGTCTGCAACCTGAAGATTTTGCAACTTACTACTGTCAGCAAGCATACAGTG |
| | | TCCCTATCACTTTTGGCGGAGGGACCAAGGTTGAGATCAAA |
| 737 | BIIB-12-1331_VL | EIVLTQSPAKKSLSPGERATLSCRASQSVSSYLAWYQQKPGQAPRLLIYDASNRATGIPARFSGSGSGTD |
| | Amino Acid | FTLTISSLEPEDFAVYYCQQRVNLPITFGGGTKVVFK |
| | Sequence | |
| 738 | BIIB-12-1331_VL | GAAATTGTGTTGACACAGTCTCCAGCCAAAAAGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGAGAGTCAATC |
| | | TCCCTATCACTTTTGGCGGAGGGACCAAGGTTGTGTTCAAA |
| 739 | BIIB-12-1332_VL | DIQMTQSPSSVSASVGDRVTITCRASQGISSWLAWYQQKPGKAPKLLIYAASSLQSGVPSRFSGSGSGTD |
| | Amino Acid | FTLTISSLQPEDFATYYCQQVNSFPLTFGGGTKVEFK |
| | Sequence | |
| 740 | BIIB-12-1332_VL | GACATCCAGATGACCCAGTCTCCATCTTCCGTATCAGCATCTGTAGGAGACAGAGTCACCATCACTTGTC |
| | Nucleic Acid | GGGCGAGTCAGGGTATTAGCAGCTGGTTAGCCTGGTATCAGCAGAAACCAGGGAAAGCCCCTAAGCTCCT |
| | Sequence | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGTAAACAGTT |
| | | TCCCTCTCACTTTTGGCGGAGGGACCAAGGTTGAGTTCAAA |
| 741 | BIIB-12-1333_VL | EIVMTQSPATLSVSPGERATLSCRASQSVSSNLAWYQQKPGQAPRLLIYGASTRATGIPARFSGSGSGTE |
| | Amino Acid | FTLTISSLQSEDFAVYYCQQVNTWPTFGGGTKVEIK |
| | Sequence | |
| 742 | BIIB-12-1333_VL | GAAATAGTGATGACGCAGTCTCCAGCCACCCTGTCTGTGTCTCCAGGGGAAAGAGCCACCCTCTCCTGCA |
| | Nucleic Acid | GGGCCAGTCAGAGTGTTAGCAGCAACTTAGCCTGGTACCAGCAGAAACCTGGCCAGGCTCCCAGGCTCCT |
| | Sequence | CATCTATGGTGCATCCACCAGGGCCACTGGTATCCCAGCCAGGTTCAGTGGCAGTGGGTCTGGGACAGAG |
| | | TTCACTCTCACCATCAGCAGCCTGCAGTCTGAAGATTTTGCAGTTTATTACTGTCAGCAGGTCAATACCT |
| | | GGCCTACTTTTGGCGGAGGGACCAAGGTTGAGATCAAA |
| 743 | BIIB-12-1334_VL | DIVMTQSPLSLPVTPGEPASISCRSSQSLLHSNGYNYLDWYLQKPGQSPQLLIYLASNRASGVPDRFSGS |
| | Amino Acid | GSGTDFTLKISRVEAEDVGVYYCMQRIGTPWTFGGGTKVEIK |
| | Sequence | |
| 744 | BIIB-12-1334_VL | GATATTGTGATGACTCAGTCTCCACTCTCCCTGCCCGTCACCCCTGGAGAGCCGGCCTCCATCTCCTGCA |
| | Nucleic Acid | GGTCTAGTCAGAGCCTCCTGCATAGTAATGGATACAACTATTTGGATTGGTACCTGCAGAAGCCAGGGCA |
| | Sequence | GTCTCCACAGCTCCTGATCTATTTGGCTTCTAATCGGGCCTCCGGGGTCCCTGACAGGTTCAGTGGCAGT |
| | | GGATCAGGCACAGATTTTACACTGAAAATCAGCAGAGTGGAGGCTGAGGATGTTGGGGTTTATTACTGCA |
| | | TGCAGAGAATAGGCACTCCTTGGACTTTTGGCGGAGGGACCAAGGTTGAGATCAAA |

Class VI Antibodies - VL Sequences

| | | |
|---|---|---|
| 745 | BIIB-12-894_VL | DIQMTQSPSSLSASVGDRVTITCQASQDISNYLNWYQQKPGKAPKLLIYDASNLETGVPSRFSGSGSGTD |
| | Amino Acid | FTFTISSLQPEDIATYYCQQDDALPFTFGGGTKVEI |
| | Sequence | |
| 746 | BIIB-12-894_VL | GACATCCAGATGACCCAGTCTCCATCCTCCCTGTCTGCATCTGTAGGAGACAGAGTCACCATCACTTGCC |
| | Nucleic Acid | AGGCGAGTCAGGACATTAGCAACTATTTAAATTGGTATCAGCAGAAACCAGGGAAAGCCCCTAAGCTCCT |
| | Sequence | GATCTACGATGCATCCAATTTGGAAACAGGGGTCCCATCAAGGTTCAGTGGAAGTGGATCTGGGACAGAT |
| | | TTTACTTTCACCATCAGCAGCCTGCAGCCTGAAGATATTGCAACATATTACTGTCAGCAGGACGATGCCC |
| | | TCCCTTTCACTTTTGGCGGAGGGACCAAGGTTGAGATCAA |
| 747 | BIIB-12-925_VL | EIVLTQSPGTLSLSPGERATLSCRASQSVSSSYLAWYQQKPGQAPRLLIYGASSRATGIPDRFSGSGSGT |
| | Amino Acid | DFTLTISRLEPEDFAVYYCQQSGGSPLTFGGGTKVEIK |
| | Sequence | |
| 748 | BIIB-12-925_VL | GAAATTGTGTTGACGCAGTCTCCAGGCACCCTGTCTTTGTCTCCAGGGGAAAGAGCCACCCTCTCCTGCA |
| | Nucleic Acid | GGGCCAGTCAGAGTGTTAGCAGCAGCTACTTAGCCTGGTACCAGCAGAAACCTGGCCAGGCTCCCAGGCT |
| | Sequence | CCTCATCTATGGTGCATCCAGCAGGGCCACTGGCATCCCAGACAGGTTCAGTGGCAGTGGGTCTGGGACA |
| | | GACTTCACTCTCACCATCAGCAGACTGGAGCCTGAAGATTTTGCAGTGTATTACTGTCAGCAGTCCGGAG |
| | | GCTCCCCTCTCACTTTTGGCGGAGGGACCAAGGTTGAGATCAAA |
| 749 | BIIB-12-1320_VL | DIQMTQSPSSLSASVGDRVTITCRASQSISSYLNWYQQKPGKAPKLLIYAASSLQSGVPSRFSGSGSGTD |
| | Amino Acid | FTLTISSLQPEDFATYYCQQADDTPWTFGGGTKVEIK |
| | Sequence | |
| 750 | BIIB-12-1320_VL | GACATCCAGATGACCCAGTCTCCATCCTCCCTGTCTGCATCTGTAGGAGACAGAGTCACCATCACTTGCC |
| | Nucleic Acid | GGGCAAGTCAGAGCATTAGCAGCTATTTAAATTGGTATCAGCAGAAACCAGGGAAAGCCCCTAAGCTCCT |
| | Sequence | GATCTATGCTGCATCCAGTTTGCAAAGTGGGGTCCCATCAAGGTTCAGTGGCAGTGGATCTGGGACAGAT |
| | | TTCACTCTCACCATCAGCAGTCTGCAACCTGAAGATTTTGCAACTTACTACTGTCAGCAAGCAGACGACA |
| | | CTCCTTGGACTTTTGGCGGAGGGACCAAGGTTGAGATCAAA |
| 751 | BIIB-12-1321_VL | EIVMTQSPATLSLSPGERATLSCRASQSVSSYLAWYQQKPGQAPRLLIYDSSNRATGIPARFSGSGSGTD |
| | Amino Acid | FTLTISSLEPEDFAVYYCQQFTNLPYTFGGGTKVEIK |
| | Sequence | |
| 752 | BIIB-12-1321_VL | GAAATTGTGATGACACAGTCTCCAGCCACCCTGTCTTTGTCTCCAGGGGAAAGAGCCACCCTCTCCTGCA |
| | Nucleic Acid | GGGCCAGTCAGAGTGTTAGCAGCTACTTAGCCTGGTACCAACAGAAACCTGGCCAGGCTCCCAGGCTCCT |
| | Sequence | CATCTATGATTCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGTTCACCAATC |
| | | TCCCTTACACTTTTGGCGGAGGGACCAAGGTTGAGATCAAA |
| 756 | BIIB-FIX-147a- | YNSGKLEEFVQGNLERECMEEKCSFEEAREVFENTERTTEFWKQYVDGDQCESNPCLNGGSCKDDINSYE |
| | LC Amino Acid | CWCPFGFEGKNCELDVTCNIKNGRCEQFCKNSADNKWCSCTEGYRLAENQKSCEPAVPFPCGRVSVSQTS |
| | Sequence | KLTR |
| | (Light chain | |
| | of activated | |
| | Factor IX) | |
| 757 | BIIB-FIX-147a- | TATAATTCAGGTAAATTGGAAGAGTTTGTTCAAGGGAATCTAGAGAGAGAATGTATGGAAGAAAAGTGTA |
| | LC Nucleic Acid | GTTTTGAAGAAGCACGAGAAGTTTTTGAAAACACTGAAAGAACAACTGAATTTTGGAAGCAGTATGTTGA |
| | Sequence | TGGAGATCAGTGTGAGTCCAATCCATGTTTAAATGGCGGCAGTTGCAAGGATGACATTAATTCCTATGAA |
| | | TGTTGGTGTCCCTTTGGATTTGAAGGAAAGAACTGTGAATTAGATGTAACATGTAACATTAAGAATGGCA |
| | | GATGCGAGCAGTTTTGTAAAAATAGTGCTGATAACAAGGTGGTTTGCTCCTGTACTGAGGGATATCGACT |
| | | TGCAGAAAACCAGAAGTCCTGTGAACCAGCAGTGCCATTTCCATGTGGAAGAGTTTCTGTTTCACAAACT |
| | | TCTAAGCTCACCCGT |
| 758 | BIIB-FIX-147a- HC Amino Acid Sequence (Heavy chain | 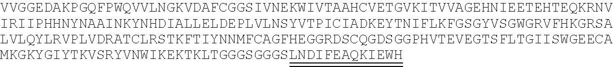 |
| | of activated | |
| | FIX) C-terminal | |
| | (G₃S)₂ linker | |
| | (underlined)and | |
| | BioTag (double | |
| | underlined) | |
| 759 | BIIB-FIX-147a- HC Nucleic Acid Sequence (Heavy chain of activated FIX) C-terminal(G₃S)₂ linker (under- lined)and BioTag (double underlined) | 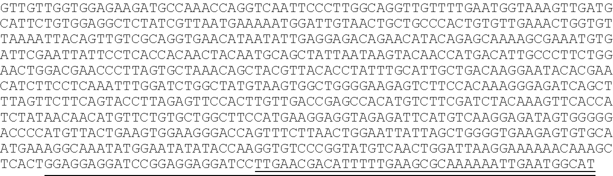 |
| 760 | BIIB-FIX-148 Amino Acid Sequence Non- activatable Factor IX construct comprising Arg | 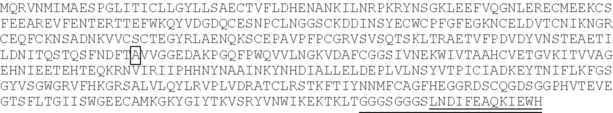 |
| | to Ala mutation | |
| | (boxed) to | |
| | prevent acti- | |
| | vation of FIX | |
| | and C-terminal | |
| | (G₃S)₂ linker | |
| | (underlined) | |
| | and BioTag | |
| | (double | |
| | underlined) | |
| 761 | BIIB-FIX-148 Nucleic Acid Sequence Non- activatable Factor X construct comprising Arg to Ala mutation(boxed) to prevent activation of FX, and C- terminal (G₃S)₂ linker (under- lined) and BioTag (double underlined) | 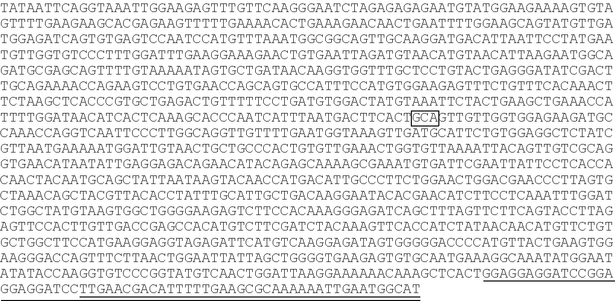 |
| 762 | BIIB-FX-015 Amino Acid Sequence | 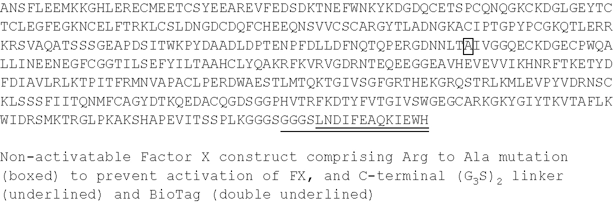 |
| 763 | BIIB-FX-015 Nucleic Acid Sequence Non- activatable Factor X construct comprising Arg to Ala mutation (boxed) to prevent acti- vation of FX, and C-terminal (G₃S)₂ linker (underlined) and BioTag (double underlined) | 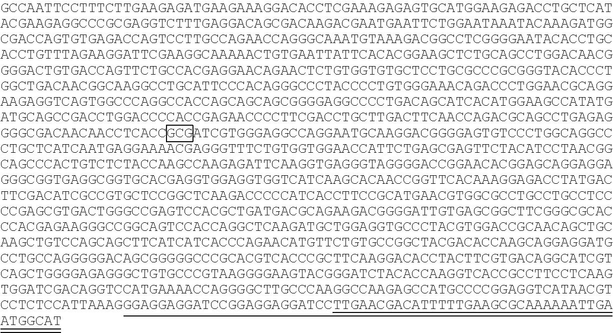 |
| 764 | pre-pro-FIX | MQRVNMIMAESPGLITICLLGYLLSAECTVFLDHENANKILNRPKRYNSGKLEEFVQGNLERECMEEKCS |
| | zymogen | FEEAREVFENTERTTEFWKQYVDGDQCESNPCLNGGSCKDDINSYECWCPFGFEGKNCELDVTCNIKNGR |
| | | CEQFCKNSADNKVVCSCTEGYRLAENQKSCEPAVPFPCGRVSVSQTSKLTRAETVFPDVDYVNSTEAETI |
| | | LDNITQSTQSFNDFTRVVGGEDAKPGQFPWQVVLNGKVDAFCGGSIVNEKWIVTAAHCVETGVKITWAGE |
| | | HNIEETEHTEQKRNVIRIIPHHNYNAAINKYNHDIALLELDEPLVLNSYVTPICIADKEYTNIFLKFGSG |
| | | YVSGWGRVFHKGRSALVLQYLRVPLVDRATCLRSTKFTIYNNMFCAGFHEGGRDSCQGDSGGPHVTEVEG |
| | | TSFLTGIISWGEECAMKGKYGIYTKVSRYVNWIKEKTKLT |

TABLE 5
SEQ ID NO Designation for CDRs.

| | | | | | | |
|---|---|---|---|---|---|---|
| Antibody | VH-CDR1 | VH-CDR2 | VH-CDR3 | VL-CDR1 | VL-CDR2 | VL-CDR3 |

Class I antibodies

| | | | | | | |
|---|---|---|---|---|---|---|
| BIIB-9-605 | 800 | 845 | 890 | 935 | 980 | 1025 |
| BIIB-9-475 | 801 | 846 | 891 | 936 | 981 | 1026 |
| BIIB-9-477 | 802 | 847 | 892 | 937 | 982 | 1027 |
| BIIB-9-479 | 803 | 848 | 893 | 938 | 983 | 1028 |
| BIIB-9-480 | 804 | 849 | 894 | 939 | 984 | 1029 |
| BIIB-9-558 | 805 | 850 | 895 | 940 | 985 | 1030 |
| BIIB-9-414 | 806 | 851 | 896 | 941 | 986 | 1031 |
| BIIB-9-415 | 807 | 852 | 897 | 942 | 987 | 1032 |
| BIIB-9-425 | 808 | 853 | 898 | 943 | 988 | 1033 |
| BIIB-9-440 | 809 | 854 | 899 | 944 | 989 | 1034 |
| BIIB-9-452 | 810 | 855 | 900 | 945 | 990 | 1035 |
| BIIB-9-460 | 811 | 856 | 901 | 946 | 991 | 1036 |
| BIIB-9-461 | 812 | 857 | 902 | 947 | 992 | 1037 |
| BIIB-9-465 | 813 | 858 | 903 | 948 | 993 | 1038 |
| BIIB-9-564 | 814 | 859 | 904 | 949 | 994 | 1039 |
| BIIB-9-484 | 815 | 860 | 905 | 950 | 995 | 1040 |
| BIIB-9-469 | 816 | 861 | 906 | 951 | 996 | 1041 |
| BIIB-9-566 | 817 | 862 | 907 | 952 | 997 | 1042 |
| BIIB-9-567 | 818 | 863 | 908 | 953 | 998 | 1043 |
| BIIB-9-569 | 819 | 864 | 909 | 954 | 999 | 1044 |
| BIIB-9-588 | 820 | 865 | 910 | 955 | 1000 | 1045 |
| BIIB-9-611 | 821 | 866 | 911 | 956 | 1001 | 1046 |
| BIIB-9-619 | 822 | 867 | 912 | 957 | 1002 | 1047 |
| BIIB-9-626 | 823 | 868 | 913 | 958 | 1003 | 1048 |
| BIIB-9-883 | 824 | 869 | 914 | 959 | 1004 | 1049 |
| BIIB-9-419 | 825 | 870 | 915 | 960 | 1005 | 1050 |
| BIIB-9-451 | 826 | 871 | 916 | 961 | 1006 | 1051 |
| BIIB-9-473 | 827 | 872 | 917 | 962 | 1007 | 1052 |
| BIIB-9-565 | 828 | 873 | 918 | 963 | 1008 | 1053 |
| BIIB-9-573 | 829 | 874 | 919 | 964 | 1009 | 1054 |
| BIIB-9-579 | 830 | 875 | 920 | 965 | 1010 | 1055 |
| BIIB-9-581 | 831 | 876 | 921 | 966 | 1011 | 1056 |
| BIIB-9-582 | 832 | 877 | 922 | 967 | 1012 | 1057 |
| BIIB-9-585 | 833 | 878 | 923 | 968 | 1013 | 1058 |
| BIIB-9-587 | 834 | 879 | 924 | 969 | 1014 | 1059 |
| BIIB-9-590 | 835 | 880 | 925 | 970 | 1015 | 1060 |
| BIIB-9-592 | 836 | 881 | 926 | 971 | 1016 | 1061 |
| BIIB-9-606 | 837 | 882 | 927 | 972 | 1017 | 1062 |
| BIIB-9-608 | 838 | 883 | 928 | 973 | 1018 | 1063 |
| BIIB-9-616 | 839 | 884 | 929 | 974 | 1019 | 1064 |
| BIIB-9-621 | 840 | 885 | 930 | 975 | 1020 | 1065 |
| BIIB-9-622 | 841 | 886 | 931 | 976 | 1021 | 1066 |
| BIIB-9-627 | 842 | 887 | 932 | 977 | 1022 | 1067 |
| BIIB-9-1335 | 843 | 888 | 933 | 978 | 1023 | 1068 |
| BIIB-9-1336 | 844 | 889 | 934 | 979 | 1024 | 1069 |

Class II Antibodies

| | | | | | | |
|---|---|---|---|---|---|---|
| BIIB-9-408 | 1070 | 1074 | 1078 | 1082 | 1086 | 1090 |
| BIIB-9-416 | 1071 | 1075 | 1079 | 1083 | 1087 | 1091 |
| BIIB-9-629 | 1072 | 1076 | 1080 | 1084 | 1088 | 1092 |
| BIIB-9-885 | 1073 | 1077 | 1081 | 1085 | 1089 | 1093 |

Class III Antibodies

| | | | | | | |
|---|---|---|---|---|---|---|
| BIIB-9-607 | 1094 | 1136 | 1178 | 1220 | 1262 | 1304 |
| BIIB-9-471 | 1095 | 1137 | 1179 | 1221 | 1263 | 1305 |
| BIIB-9-472 | 1096 | 1138 | 1180 | 1222 | 1264 | 1306 |
| BIIB-9-439 | 1097 | 1139 | 1181 | 1223 | 1265 | 1307 |
| BIIB-9-446 | 1098 | 1140 | 1182 | 1224 | 1266 | 1308 |
| BIIB-9-568 | 1099 | 1141 | 1183 | 1225 | 1267 | 1309 |
| BIIB-9-615 | 1100 | 1142 | 1184 | 1226 | 1268 | 1310 |
| BIIB-9-628 | 1101 | 1143 | 1185 | 1227 | 1269 | 1311 |
| BIIB-9-882 | 1102 | 1144 | 1186 | 1228 | 1270 | 1312 |
| BIIB-9-884 | 1103 | 1145 | 1187 | 1229 | 1271 | 1313 |
| BIIB-9-886 | 1104 | 1146 | 1188 | 1230 | 1272 | 1314 |
| BIIB-9-887 | 1105 | 1147 | 1189 | 1231 | 1273 | 1315 |
| BIIB-9-888 | 1106 | 1148 | 1190 | 1232 | 1274 | 1316 |
| BIIB-9-889 | 1107 | 1149 | 1191 | 1233 | 1275 | 1317 |
| BIIB-9-433 | 1108 | 1150 | 1192 | 1234 | 1276 | 1318 |
| BIIB-9-445 | 1109 | 1151 | 1193 | 1235 | 1277 | 1319 |
| BIIB-9-470 | 1110 | 1152 | 1194 | 1236 | 1278 | 1320 |
| BIIB-9-625 | 1111 | 1153 | 1195 | 1237 | 1279 | 1321 |
| BIIB-9-1264 | 1112 | 1154 | 1196 | 1238 | 1280 | 1322 |
| BIIB-9-1265 | 1113 | 1155 | 1197 | 1239 | 1281 | 1323 |
| BIIB-9-1266 | 1114 | 1156 | 1198 | 1240 | 1282 | 1324 |
| BIIB-9-1267 | 1115 | 1157 | 1199 | 1241 | 1283 | 1325 |
| BIIB-9-1268 | 1116 | 1158 | 1200 | 1242 | 1284 | 1326 |
| BIIB-9-1269 | 1117 | 1159 | 1201 | 1243 | 1285 | 1327 |
| BIIB-9-1270 | 1118 | 1160 | 1202 | 1244 | 1286 | 1328 |
| BIIB-9-1271 | 1119 | 1161 | 1203 | 1245 | 1287 | 1329 |
| BIIB-9-1272 | 1120 | 1162 | 1204 | 1246 | 1288 | 1330 |
| BIIB-9-1273 | 1121 | 1163 | 1205 | 1247 | 1289 | 1331 |
| BIIB-9-1274 | 1122 | 1164 | 1206 | 1248 | 1290 | 1332 |
| BIIB-9-1275 | 1123 | 1165 | 1207 | 1249 | 1291 | 1333 |
| BIIB-9-1276 | 1124 | 1166 | 1208 | 1250 | 1292 | 1334 |
| BIIB-9-1277 | 1125 | 1167 | 1209 | 1251 | 1293 | 1335 |
| BIIB-9-1278 | 1126 | 1168 | 1210 | 1252 | 1294 | 1336 |
| BIIB-9-1279 | 1127 | 1169 | 1211 | 1253 | 1295 | 1337 |
| BIIB-9-1280 | 1128 | 1170 | 1212 | 1254 | 1296 | 1338 |
| BIIB-9-1281 | 1129 | 1171 | 1213 | 1255 | 1297 | 1339 |
| BIIB-9-1282 | 1130 | 1172 | 1214 | 1256 | 1298 | 1340 |
| BIIB-9-1283 | 1131 | 1173 | 1215 | 1257 | 1299 | 1341 |
| BIIB-9-1284 | 1132 | 1174 | 1216 | 1258 | 1300 | 1342 |
| BIIB-9-1285 | 1133 | 1175 | 1217 | 1259 | 1301 | 1343 |
| BIIB-9-1286 | 1134 | 1176 | 1218 | 1260 | 1302 | 1344 |
| BIIB-9-1287 | 1135 | 1177 | 1219 | 1261 | 1303 | 1345 |

Class IV Antibodies

| | | | | | | |
|---|---|---|---|---|---|---|
| BIIB-9-397 | 1346 | 1350 | 1354 | 1358 | 1362 | 1366 |
| BIIB-9-578 | 1347 | 1351 | 1355 | 1359 | 1363 | 1367 |
| BIIB-9-631 | 1348 | 1352 | 1356 | 1360 | 1364 | 1368 |
| BIIB-9-612 | 1349 | 1353 | 1357 | 1361 | 1365 | 1369 |

Class V Antibodies

| | | | | | | |
|---|---|---|---|---|---|---|
| BIIB-12-891 | 1370 | 1460 | 1550 | 1640 | 1730 | 1820 |
| BIIB-12-892 | 1371 | 1461 | 1551 | 1641 | 1731 | 1821 |
| BIIB-12-893 | 1372 | 1462 | 1552 | 1642 | 1732 | 1822 |
| BIIB-12-895 | 1373 | 1463 | 1553 | 1643 | 1733 | 1823 |
| BIIB-12-896 | 1374 | 1464 | 1554 | 1644 | 1734 | 1824 |
| BIIB-12-897 | 1375 | 1465 | 1555 | 1645 | 1735 | 1825 |
| BIIB-12-898 | 1376 | 1466 | 1556 | 1646 | 1736 | 1826 |
| BIIB-12-899 | 1377 | 1467 | 1557 | 1647 | 1737 | 1827 |
| BIIB-12-900 | 1378 | 1468 | 1558 | 1648 | 1738 | 1828 |
| BIIB-12-901 | 1379 | 1469 | 1559 | 1649 | 1739 | 1829 |
| BIIB-12-902 | 1380 | 1470 | 1560 | 1650 | 1740 | 1830 |
| BIIB-12-903 | 1381 | 1471 | 1561 | 1651 | 1741 | 1831 |
| BIIB-12-904 | 1382 | 1472 | 1562 | 1652 | 1742 | 1832 |
| BIIB-12-905 | 1383 | 1473 | 1563 | 1653 | 1743 | 1833 |
| BIIB-12-906 | 1384 | 1474 | 1564 | 1654 | 1744 | 1834 |
| BIIB-12-907 | 1385 | 1475 | 1565 | 1655 | 1745 | 1835 |
| BIIB-12-908 | 1386 | 1476 | 1566 | 1656 | 1746 | 1836 |
| BIIB-12-909 | 1387 | 1477 | 1567 | 1657 | 1747 | 1837 |
| BIIB-12-910 | 1388 | 1478 | 1568 | 1658 | 1748 | 1838 |
| BIIB-12-911 | 1389 | 1479 | 1569 | 1659 | 1749 | 1839 |
| BIIB-12-912 | 1390 | 1480 | 1570 | 1660 | 1750 | 1840 |
| BIIB-12-913 | 1391 | 1481 | 1571 | 1661 | 1751 | 1841 |
| BIIB-12-914 | 1392 | 1482 | 1572 | 1662 | 1752 | 1842 |
| BIIB-12-915 | 1393 | 1483 | 1573 | 1663 | 1753 | 1843 |
| BIIB-12-916 | 1394 | 1484 | 1574 | 1664 | 1754 | 1844 |
| BIIB-12-917 | 1395 | 1485 | 1575 | 1665 | 1755 | 1845 |
| BIIB-12-918 | 1396 | 1486 | 1576 | 1666 | 1756 | 1846 |
| BIIB-12-919 | 1397 | 1487 | 1577 | 1667 | 1757 | 1847 |
| BIIB-12-920 | 1398 | 1488 | 1578 | 1668 | 1758 | 1848 |
| BIIB-12-921 | 1399 | 1489 | 1579 | 1669 | 1759 | 1849 |
| BIIB-12-922 | 1400 | 1490 | 1580 | 1670 | 1760 | 1850 |
| BIIB-12-923 | 1401 | 1491 | 1581 | 1671 | 1761 | 1851 |
| BIIB-12-924 | 1402 | 1492 | 1582 | 1672 | 1762 | 1852 |
| BIIB-12-926 | 1403 | 1493 | 1583 | 1673 | 1763 | 1853 |
| BIIB-12-927 | 1404 | 1494 | 1584 | 1674 | 1764 | 1854 |
| BIIB-12-928 | 1405 | 1495 | 1585 | 1675 | 1765 | 1855 |
| BIIB-12-929 | 1406 | 1496 | 1586 | 1676 | 1766 | 1856 |
| BIIB-12-930 | 1407 | 1497 | 1587 | 1677 | 1767 | 1857 |
| BIIB-12-931 | 1408 | 1498 | 1588 | 1678 | 1768 | 1858 |
| BIIB-12-932 | 1409 | 1499 | 1589 | 1679 | 1769 | 1859 |
| BIIB-12-933 | 1410 | 1500 | 1590 | 1680 | 1770 | 1860 |
| BIIB-12-934 | 1411 | 1501 | 1591 | 1681 | 1771 | 1861 |
| BIIB-12-935 | 1412 | 1502 | 1592 | 1682 | 1772 | 1862 |
| BIIB-12-936 | 1413 | 1503 | 1593 | 1683 | 1773 | 1863 |
| BIIB-12-937 | 1414 | 1504 | 1594 | 1684 | 1774 | 1864 |
| BIIB-12-1288 | 1415 | 1505 | 1595 | 1685 | 1775 | 1865 |
| BIIB-12-1289 | 1416 | 1506 | 1596 | 1686 | 1776 | 1866 |
| BIIB-12-1290 | 1417 | 1507 | 1597 | 1687 | 1777 | 1867 |
| BIIB-12-1291 | 1418 | 1508 | 1598 | 1688 | 1778 | 1868 |
| BIIB-12-1292 | 1419 | 1509 | 1599 | 1689 | 1779 | 1869 |
| BIIB-12-1293 | 1420 | 1510 | 1600 | 1690 | 1780 | 1870 |
| BIIB-12-1294 | 1421 | 1511 | 1601 | 1691 | 1781 | 1871 |
| BIIB-12-1295 | 1422 | 1512 | 1602 | 1692 | 1782 | 1872 |
| BIIB-12-1296 | 1423 | 1513 | 1603 | 1693 | 1783 | 1873 |
| BIIB-12-1297 | 1424 | 1514 | 1604 | 1694 | 1784 | 1874 |
| BIIB-12-1298 | 1425 | 1515 | 1605 | 1695 | 1785 | 1875 |
| BIIB-12-1299 | 1426 | 1516 | 1606 | 1696 | 1786 | 1876 |
| BIIB-12-1300 | 1427 | 1517 | 1607 | 1697 | 1787 | 1877 |
| BIIB-12-1301 | 1428 | 1518 | 1608 | 1698 | 1788 | 1878 |
| BIIB-12-1302 | 1429 | 1519 | 1609 | 1699 | 1789 | 1879 |
| BIIB-12-1303 | 1430 | 1520 | 1610 | 1700 | 1790 | 1880 |
| BIIB-12-1304 | 1431 | 1521 | 1611 | 1701 | 1791 | 1881 |
| BIIB-12-1305 | 1432 | 1522 | 1612 | 1702 | 1792 | 1882 |
| BIIB-12-1306 | 1433 | 1523 | 1613 | 1703 | 1793 | 1883 |
| BIIB-12-1307 | 1434 | 1524 | 1614 | 1704 | 1794 | 1884 |
| BIIB-12-1308 | 1435 | 1525 | 1615 | 1705 | 1795 | 1885 |
| BIIB-12-1309 | 1436 | 1526 | 1616 | 1706 | 1796 | 1886 |
| BIIB-12-1310 | 1437 | 1527 | 1617 | 1707 | 1797 | 1887 |
| BIIB-12-1311 | 1438 | 1528 | 1618 | 1708 | 1798 | 1888 |
| BIIB-12-1312 | 1439 | 1529 | 1619 | 1709 | 1799 | 1889 |
| BIIB-12-1313 | 1440 | 1530 | 1620 | 1710 | 1800 | 1890 |
| BIIB-12-1314 | 1441 | 1531 | 1621 | 1711 | 1801 | 1891 |
| BIIB-12-1315 | 1442 | 1532 | 1622 | 1712 | 1802 | 1892 |
| BIIB-12-1316 | 1443 | 1533 | 1623 | 1713 | 1803 | 1893 |
| BIIB-12-1317 | 1444 | 1534 | 1624 | 1714 | 1804 | 1894 |
| BIIB-12-1318 | 1445 | 1535 | 1625 | 1715 | 1805 | 1895 |
| BIIB-12-1319 | 1446 | 1536 | 1626 | 1716 | 1806 | 1896 |
| BIIB-12-1322 | 1447 | 1537 | 1627 | 1717 | 1807 | 1897 |
| BIIB-12-1323 | 1448 | 1538 | 1628 | 1718 | 1808 | 1898 |
| BIIB-12-1324 | 1449 | 1539 | 1629 | 1719 | 1809 | 1899 |
| BIIB-12-1325 | 1450 | 1540 | 1630 | 1720 | 1810 | 1900 |
| BIIB-12-1326 | 1451 | 1541 | 1631 | 1721 | 1811 | 1901 |
| BIIB-12-1327 | 1452 | 1542 | 1632 | 1722 | 1812 | 1902 |
| BIIB-12-1328 | 1453 | 1543 | 1633 | 1723 | 1813 | 1903 |
| BIIB-12-1329 | 1454 | 1544 | 1634 | 1724 | 1814 | 1904 |
| BIIB-12-1330 | 1455 | 1545 | 1635 | 1725 | 1815 | 1905 |
| BIIB-12-1331 | 1456 | 1546 | 1636 | 1726 | 1816 | 1906 |
| BIIB-12-1332 | 1457 | 1547 | 1637 | 1727 | 1817 | 1907 |
| BIIB-12-1333 | 1458 | 1548 | 1638 | 1728 | 1818 | 1908 |
| BIIB-12-1334 | 1459 | 1549 | 1639 | 1729 | 1819 | 1909 |

Class VI Antibodies

| BIIB-12-894 | 1910 | 1914 | 1918 | 1922 | | |
|---|---|---|---|---|---|---|
| BIIB-12-925 | 1911 | 1915 | 1919 | 1923 | 1927 | 1931 |
| BIIB-12-1320 | 1912 | 1916 | 1920 | 1924 | 1928 | 1932 |
| BIIB-12-1321 | 1913 | 1917 | 1921 | 1925 | 1929 | 1933 |

TABLE 6
Sequences of Example 14 daughter antibodies

| | | |
|---|---|---|
| | Description | |
| | (Antibody, | |
| SEQ | domain, | |
| ID | sequence | |
| NO | type) | Sequence |

Parent Antibody: BIIB-9-484

| | | |
|---|---|---|
| 1934 | BIIB-9-3595 | GAGGTGCAGCTGGTGGAGTCTGGGGGAGGCCTGGTCAAGCCTGGGGGGTCCCTGAGACTCTCCTGTGCAG |
| | VH (na) | CCTCTGGATTCACCTTCAGTAGCTATAGCATGAACTGGGTCCGCCAGGCTCCAGGGAAGGGGCTGGAGTG |
| | | GGTCTCATCCATTAGTAGTAGTAGTAGTTACATATACTACGCAGACTCAGTGAAAGGCCGATTCACCATC |
| | | TCCAGAGACAACGCCAAGAACTCACTGTATCTGCAAATGAACAGCCTGAGAGCCGAGGACACGGCGGTGT |
| | | ACTACTGCGCCAGAGATTTGGGTGGATACGCAGGGTACTACGGCATGGATGTATGGGGGCAAGGGACCAC |
| | | GGTCACCGTCTCCTCA |
| 1935 | BIIB-9-3595 | EVQLVESGGGLVKPGGSLRLSCAASGFTFSSYSMNWVRQAPGKGLEWVSSISSSSSYIYYADSVKGRFTI |
| | VH (aa) | SRDNAKNSLYLQMNSLRAEDTAVYYCARDLGGYAGYYGMDVWGQGTTVTVSS |
| 1936 | BIIB-9-3595 | GACATCCAGATGACCCAGTCTCCATCCTCCCTGTCTGCATCTGTAGGAGACAGAGTCACCATCACTTGCC |
| | VL (na) | AGGCGAGTCAGGACATTGCCAACTATTTAAATTGGTATCAGCAGAAACCAGGGAAAGCCCCTAAGCTCCT |
| | | GATCTACGATGCATCCAATTTGGAAACAGGGGTCCCATCAAGGTTCAGTGGAAGTGGATCTGGGACAGAT |
| | | TTTACTTTCACCATCAGCAGCCTGCAGCCTGAAGATATTGCAACATATTACTGTCAGCAGTACGCCAACT |
| | | TCCCTTACACTTTTGGCGGAGGGACCAAGGTTGAGATCAAA |
| 1937 | BIIB-9-3595 | DIQMTQSPSSLSASVGDRVTITCQASQDIANYLNWYQQKPGKAPKLLIYDASNLETGVPSRFSGSGSGTD |
| | VL (aa) | FTFTISSLQPEDIATYYCQQYANFPYTFGGGTKVEIK |
| 1938 | BIIB-9-3601 | GAGGTGCAGCTGGTGGAGTCTGGGGGAGGCCTGGTCAAGCCTGGGGGGTCCCTGAGACTCTCCTGTGCAG |
| | VH (na) | CCTCTGGATTCACCTTCAGTAGCTTCAGCATGAACTGGGTCCGCCAGGCTCCAGGGAAGGGGCTGGAGTG |
| | | GGTCTCATCCATTAGTAGTGCTGGGAGTTACATATACTACGCAGACTCAGTGAAGGGCCGATTCACCATC |
| | | TCCAGAGACAACGCCAAGAACTCACTGTATCTGCAAATGAACAGCCTGAGAGCCGAGGACACGGCGGTGT |
| | | ACTACTGCGCCAGAGATGTAGGAGGATACGCAGGGTACTACGGCATGGATGTATGGGGCCAGGGAACAAC |
| | | TGTCACCGTCTCCTCA |
| 1939 | BIIB-9-3601 | EVQLVESGGGLVKPGGSLRLSCAASGFTFSSFSMNWVRQAPGKGLEWVSSISSAGSYIYYADSVKGRFTI |
| | VH (aa) | SRDNAKNSLYLQMNSLRAEDTAVYYCARDVGGYAGYYGMDVWGQGTTVTVSS |
| 1940 | BIIB-9-3601 | GACATCCAGATGACCCAGTCTCCATCCTCCCTGTCTGCATCTGTAGGAGACAGAGTCACCATCACTTGCC |
| | VL (na) | AGGCGAGTCAGGACATTGCCAACTATTTAAATTGGTATCAGCAGAAACCAGGGAAAGCCCCTAAGCTCCT |
| | | GATCTACGATGCATCCAATTTGGAAACAGGGGTCCCATCAAGGTTCAGTGGAAGTGGATCTGGGACAGAT |
| | | TTTACTTTCACCATCAGCAGCCTGCAGCCTGAAGATATTGCAACATATTACTGTCAGCAGTACGCCAACT |
| | | TCCCTTACACTTTTGGCGGAGGGACCAAGGTTGAGATCAAA |
| 1941 | BIIB-9-3601 | DIQMTQSPSSLSASVGDRVTITCQASQDIANYLNWYQQKPGKAPKLLIYDASNLETGVPSRFSGSGSGTD |
| | VL faa) | FTFTISSLQPEDIATYYCQQYANFPYTFGGGTKVEIK |
| 1942 | BIIB-9-3604 | GAGGTGCAGCTGGTGGAGTCTGGGGGAGGCCTGGTCAAGCCTGGGGGGTCCCTGAGACTCTCCTGTGCAG |
| | VH (na) | CCTCTGGATTCACCTTCAGTAGCTATGATATGGTGTGGGTCCGCCAGGCTCCAGGGAAGGGGCTGGAGTG |
| | | GGTCTCATCCATTAGTAGTGGGGATAGTTACATATACTACGCAGACTCAGTGAAGGGCCGATTCACCATC |
| | | TCCAGAGACAACGCCAAGAACTCACTGTATCTGCAAATGAACAGCCTGAGAGCCGAGGACACGGCGGTGT |
| | | ACTACTGCGCCAGAGATGTAGGAGGATACGCAGGGTACTACGGCATGGATGTATGGGGCCAGGGAACAAC |
| | | TGTCACCGTCTCCTCA |
| 1943 | BIIB-9-3604 | EVQLVESGGGLVKPGGSLRLSCAASGFTFSSYDMVWVRQAPGKGLEWVSSISSGDSYIYYADSVKGRFTI |
| | VH (aa) | SRDNAKNSLYLQMNSLRAEDTAVYYCARDVGGYAGYYGMDVWGQGTTVTVSS |
| 1944 | BIIB-9-3604 | GACATCCAGATGACCCAGTCTCCATCCTCCCTGTCTGCATCTGTAGGAGACAGAGTCACCATCACTTGCC |
| | VL (na) | AGGCGAGTCAGGACATTGCCAACTATTTAAATTGGTATCAGCAGAAACCAGGGAAAGCCCCTAAGCTCCT |
| | | GATCTACGATGCATCCAATTTGGAAACAGGGGTCCCATCAAGGTTCAGTGGAAGTGGATCTGGGACAGAT |
| | | TTTACTTTCACCATCAGCAGCCTGCAGCCTGAAGATATTGCAACATATTACTGTCAGCAGTACGCCAACT |
| | | TCCCTTACACTTTTGGCGGAGGGACCAAGGTTGAGATCAAA |
| 1945 | BIIB-9-3604 | DIQMTQSPSSLSASVGDRVTITCQASQDIANYLNWYQQKPGKAPKLLIYDASNLETGVPSRFSGSGSGTD |
| | VL (aa) | FTFTISSLQPEDIATYYCQQYANFPYTFGGGTKVEIK |
| 1946 | BIIB-9-3617 | GAGGTGCAGCTGGTGGAGTCTGGGGGAGGCCTGGTCAAGCCTGGGGGGTCCCTGAGACTCTCCTGTGCAG |
| | VH (na) | CCTCTGGATTCACCTTCAGTAGCTATTCTATGACTTGGGTCCGCCAGGCTCCAGGGAAGGGGCTGGAGTG |
| | | GGTCTCATCCATTAGTAGTAGTGGTACGTACATATACTACGCAGACTCAGTGAAGGGCCGATTCACCATC |
| | | TCCAGAGACAACGCCAAGAACTCACTGTATCTGCAAATGAACAGCCTGAGAGCCGAGGACACGGCGGTGT |
| | | ACTACTGCGCCAGAGATGTAGGAGGATACGCAGGGTACTACGGCATGGATGTATGGGGCCAGGGAACAAC |
| | | TGTCACCGTCTCCTCA |
| 1947 | BIIB-9-3617 | EVQLVESGGGLVKPGGSLRLSCAASGFTFSSYSMTWVRQAPGKGLEWVSSISSSGTYIYYADSVKGRFTI |
| | VH (aa) | SRDNAKNSLYLQMNSLRAEDTAVYYCARDVGGYAGYYGMDVWGQGTTVTVSS |
| 1948 | BIIB-9-3617 | GACATCCAGATGACCCAGTCTCCATCCTCCCTGTCTGCATCTGTAGGAGACAGAGTCACCATCACTTGCC |
| | VL (na) | AGGCGAGTCAGGACATTGCCAACTATTTAAATTGGTATCAGCAGAAACCAGGGAAAGCCCCTAAGCTCCT |
| | | GATCTACGATGCATCCAATTTGGAAACAGGGGTCCCATCAAGGTTCAGTGGAAGTGGATCTGGGACAGAT |
| | | TTTACTTTCACCATCAGCAGCCTGCAGCCTGAAGATATTGCAACATATTACTGTCAGCAGTACGCCAACT |
| | | TCCCTTACACTTTTGGCGGAGGGACCAAGGTTGAGATCAAA |
| 1949 | BIIB-9-3617 | DIQMTQSPSSLSASVGDRVTITCQASQDIANYLNWYQQKPGKAPKLLIYDASNLETGVPSRFSGSGSGTD |
| | VL (aa) | FTFTISSLQPEDIATYYCQQYANFPYTFGGGTKVEIK |
| 1950 | BIIB-9-3618 | GAGGTGCAGCTGGTGGAGTCTGGGGGAGGCCTGGTCAAGCCTGGGGGGTCCATGAGACTCTCCTGTGCAG |
| | VH (na) | CCTCTGGATTCACCTTCAGTAGCTATGAGATGGTTTGGGTCCGCCAGGCTCCAGGGAAGGGGCTGGAGTG |
| | | GGTCTCATATATTAGTAGTGGTAGTAGTTACATATACTACGCAGACTCAGTGAAGGGCCGATTCACCATC |
| | | TCCAGAGACAACGCCAAGAACTCACTGTATCTGCAAATGAACAGCCTGAGAGCCGAGGACACGGCGGTGT |
| | | ACTACTGCGCCAGAGATGTAGGAGGATACGCAGGGTACTACGGCATGGATGTATGGGGCCAGGGAACAAC |
| | | TGTCACCGTCTCCTCA |
| 1951 | BIIB-9-3618 | EVQLVESGGGLVKPGGSMRLSCAASGFTFSSYEMVWVRQAPGKGLEWVSYISSGSSYIYYADSVKGRFTI |
| | VH (aa) | SRDNAKNSLYLQMNSLRAEDTAVYYCARDVGGYAGYYGMDVWGQGTTVTVSS |
| 1952 | BIIB-9-3618 | GACATCCAGATGACCCAGTCTCCATCCTCCCTGTCTGCATCTGTAGGAGACAGAGTCACCATCACTTGCC |
| | VL (na) | AGGCGAGTCAGGACATTGCCAACTATTTAAATTGGTATCAGCAGAAACCAGGGAAAGCCCCTAAGCTCCT |
| | | GATCTACGATGCATCCAATTTGGAAACAGGGGTCCCATCAAGGTTCAGTGGAAGTGGATCTGGGACAGAT |
| | | TTTACTTTCACCATCAGCAGCCTGCAGCCTGAAGATATTGCAACATATTACTGTCAGCAGTACGCCAACT |
| | | TCCCTTACACTTTTGGCGGAGGGACCAAGGTTGAGATCAAA |
| 1953 | BIIB-9-3618 | DIQMTQSPSSLSASVGDRVTITCQASQDIANYLNWYQQKPGKAPKLLIYDASNLETGVPSRFSGSGSGTD |
| | VL (aa) | FTFTISSLQPEDIATYYCQQYANFPYTFGGGTKVEIK |
| 1954 | BIIB-9-3621 | GAGGTGCAGCTGGTGGAGTCTGGGGGAGGCCTGGTCAAGCCTGGGGGGTCCCTGAGACTCTCCTGTGCAG |
| | VH (na) | CCTCTGGATTCACCTTCGGGAGCTATAGCATGGCTTGGGTCCGCCAGGCTCCAGGGAAGGGGCTGGAGTG |
| | | GGTCTCAGGTATTAGTAGTAGTAGTGGTTACATATACTACGCAGACTCAGTGAAGGGCCGATTCACCATC |
| | | TCCAGAGACAACGCCAAGAACTCACTGTATCTGCAAATGAACAGCCTGAGAGCCGAGGACACGGCGGTGT |
| | | ACTACTGCGCCAGAGATGTAGGAGGATACGCAGGGTACTACGGCATGGATGTATGGGGCCAGGGAACAAC |
| | | TGTCACCGTCTCCTCA |
| 1955 | BIIB-9-3621 | EVQLVESGGGLVKPGGSLRLSCAASGFTFGSYSMAWVRQAPGKGLEWVSGISSSSGYIYYADSVKGRFTI |
| | VH (aa) | SRDNAKNSLYLQMNSLRAEDTAVYYCARDVGGYAGYYGMDVWGQGTTVTVSS |
| 1956 | BIIB-9-3621 | GACATCCAGATGACCCAGTCTCCATCCTCCCTGTCTGCATCTGTAGGAGACAGAGTCACCATCACTTGCC |
| | VL (na) | AGGCGAGTCAGGACATTGCCAACTATTTAAATTGGTATCAGCAGAAACCAGGGAAAGCCCCTAAGCTCCT |
| | | GATCTACGATGCATCCAATTTGGAAACAGGGGTCCCATCAAGGTTCAGTGGAAGTGGATCTGGGACAGAT |
| | | TTTACTTTCACCATCAGCAGCCTGCAGCCTGAAGATATTGCAACATATTACTGTCAGCAGTACGCCAACT |
| | | TCCCTTACACTTTTGGCGGAGGGACCAAGGTTGAGATCAAA |
| 1957 | BIIB-9-3621 | DIQMTQSPSSLSASVGDRVTITCQASQDIANYLNWYQQKPGKAPKLLIYDASNLETGVPSRFSGSGSGTD |
| | VL (aa) | FTFTISSLQPEDIATYYCQQYANFPYTFGGGTKVEIK |
| 1958 | BIIB-9-3647 | GAGGTGCAGCTGGTGGAGTCTGGGGGAGGCCTGGTCAAGCCTGGGGGGTCCCTGAGACTCTCCTGTGCAG |
| | VH (na) | CCTCTGGATTCACCTTCAGTAGCTATGGTATGGTGTGGGTCCGCCAGGCTCCAGGGAAGGGGCTGGAGTG |
| | | GGTCTCATCCATTAGTAGTGCGAGTAGTTACATATACTACGCAGACTCAGTGAAGGGCCGATTCACCATC |
| | | TCCAGAGACAACGCCAAGAACTCACTGTATCTGCAAATGAACAGCCTGAGAGCCGAGGACACGGCGGTGT |
| | | ACTACTGCGCCAGAGATGTAGGAGGATACGCAGGGTACTACGGCATGGATGTATGGGGCCAGGGAACAAC |
| | | TGTCACCGTCTCCTCA |
| 1959 | BIIB-9-3647 | EVQLVESGGGLVKPGGSLRLSCAASGFTFSSYGMVWVRQAPGKGLEWVSSISSASSYIYYADSVKGRFTI |
| | VH (aa) | SRDNAKNSLYLQMNSLRAEDTAVYYCARDVGGYAGYYGMDVWGQGTTVTVSS |
| 1960 | BIIB-9-3647 | GACATCCAGATGACCCAGTCTCCATCCTCCCTGTCTGCATCTGTAGGAGACAGAGTCACCATCACTTGCC |
| | VL (na) | AGGCGAGTCAGGACATTGCCAACTATTTAAATTGGTATCAGCAGAAACCAGGGAAAGCCCCTAAGCTCCT |
| | | GATCTACGATGCATCCAATTTGGAAACAGGGGTCCCATCAAGGTTCAGTGGAAGTGGATCTGGGACAGAT |
| | | TTTACTTTCACCATCAGCAGCCTGCAGCCTGAAGATATTGCAACATATTACTGTCAGCAGTACGCCAACT |
| | | TCCCTTACACTTTTGGCGGAGGGACCAAGGTTGAGATCAAA |
| 1961 | BIIB-9-3647 | DIQMTQSPSSLSASVGDRVTITCQASQDIANYLNWYQQKPGKAPKLLIYDASNLETGVPSRFSGSGSGTD |
| | VL (aa) | FTFTISSLQPEDIATYYCQQYANFPYTFGGGTKVEIK |
| 1962 | BIIB-9-3649 | GAGGTGCAGCTGGTGGAGTCTGGGGGAGGCCTGGTCAAGCCTGGGGGGTCCCTGAGACTCTCCTGTGCAG |
| | VH (na) | CCTCTGGATTCACCTTCAGTAGCTATGGTATGGCTTGGGTCCGCCAGGCTCCAGGGAAGGGGCTGGAGTG |
| | | GGTCTCAGGTATTAGTAGTAGTTCGAGTTACATATACTACGCAGACTCAGTGAAGGGCCGATTCACCATC |
| | | TCCAGAGACAACGCCAAGAACTCACTGTATCTGCAAATGAACAGCCTGAGAGCCGAGGACACGGCGGTGT |
| | | ACTACTGCGCCAGAGATGTAGGAGGATACGCAGGGTACTACGGCATGGATGTATGGGGCCAGGGAACAAC |
| | | TGTCACCGTCTCCTCA |
| 1963 | BIIB-9-3649 | EVQLVESGGGLVKPGGSLRLSCAASGFTFSSYGMAWVRQAPGKGLEWVSGISSSSSYIYYADSVKGRFTI |
| | VH (aa) | SRDNAKNSLYLQMNSLRAEDTAVYYCARDVGGYAGYYGMDVWGQGTTVTVSS |
| 1964 | BIIB-9-3649 | GACATCCAGATGACCCAGTCTCCATCCTCCCTGTCTGCATCTGTAGGAGACAGAGTCACCATCACTTGCC |
| | VL (na) | AGGCGAGTCAGGACATTGCCAACTATTTAAATTGGTATCAGCAGAAACCAGGGAAAGCCCCTAAGCTCCT |
| | | GATCTACGATGCATCCAATTTGGAAACAGGGGTCCCATCAAGGTTCAGTGGAAGTGGATCTGGGACAGAT |
| | | TTTACTTTCACCATCAGCAGCCTGCAGCCTGAAGATATTGCAACATATTACTGTCAGCAGTACGCCAACT |
| | | TCCCTTACACTTTTGGCGGAGGGACCAAGGTTGAGATCAAA |
| 1965 | BIIB-9-3649 | DIQMTQSPSSLSASVGDRVTITCQASQDIANYLNWYQQKPGKAPKLLIYDASNLETGVPSRFSGSGSGTD |
| | VL (aa) | FTFTISSLQPEDIATYYCQQYANFPYTFGGGTKVEIK |
| 1966 | BIIB-9-3650 | GAGGTGCAGCTGGTGGAGTCTGGGGGAGGCTTGGTACAGCCTGGGGGGTCCCTGAGACTCTCCTGTGCAG |
| | VH (na) | CCTCTGGATTCACCTTCGGGAGCTATGAGATGAACTGGGTCCGCCAGGCTCCAGGGAAGGGGCTGGAGTG |
| | | GGTTTCAGCGATTAGTGCTAGTAGTAGTACCATATACTACGCAGACTCTGTGAAGGGCCGATTCACCATC |
| | | TCCAGAGACAATGCCAAGAACTCACTGTATCTGCAAATGAACAGCCTGAGAGCTGAGGACACGGCGGTGT |
| | | ACTACTGCGCCAGAGATGTAGGAGGATACGCAGGGTACTACGGCATGGATGTATGGGGCCAGGGAACAAC |
| | | TGTCACCGTCTCCTCA |
| 1967 | BIIB-9-3650 | EVQLVESGGGLVQPGGSLRLSCAASGFTFGSYEMNWVRQAPGKGLEWVSAISASSSTIYYADSVKGRFTI |
| | VH (aa) | SRDNAKNSLYLQMNSLRAEDTAVYYCARDVGGYAGYYGMDVWGQGTTVTVSS |
| 1968 | BIIB-9-3650 | GACATCCAGATGACCCAGTCTCCATCCTCCCTGTCTGCATCTGTAGGAGACAGAGTCACCATCACTTGCC |
| | VL (na) | AGGCGAGTCAGGACATTGCCAACTATTTAAATTGGTATCAGCAGAAACCAGGGAAAGCCCCTAAGCTCCT |
| | | GATCTACGATGCATCCAATTTGGAAACAGGGGTCCCATCAAGGTTCAGTGGAAGTGGATCTGGGACAGAT |
| | | TTTACTTTCACCATCAGCAGCCTGCAGCCTGAAGATATTGCAACATATTACTGTCAGCAGTACGCCAACT |
| | | TCCCTTACACTTTTGGCGGAGGGACCAAGGTTGAGATCAAA |
| 1969 | BIIB-9-3650 | DIQMTQSPSSLSASVGDRVTITCQASQDIANYLNWYQQKPGKAPKLLIYDASNLETGVPSRFSGSGSGTD |
| | VL (aa) | FTFTISSLQPEDIATYYCQQYANFPYTFGGGTKVEIK |
| 1970 | BIIB-9-3654 | GAGGTGCAGCTGGTGGAGTCTGGGGGAGGCCTGGTCAAGCCTGGGGGGTCCCTGAGACTCTCCTGTGCAG |
| | VH (na) | CCTCTGGATTCACCTTCGAGAGCTATAGCATGAACTGGGTCCGCCAGGCTCCAGGGAAGGGGCTGGAGTG |
| | | GGTCTCAGGGATTAGTAGTGCTAGTAGTTACATATACTACGCAGACTCAGTGAAGGGCCGATTCACCATC |
| | | TCCAGAGACAACGCCAAGAACTCACTGTATCTGCAAATGAACAGCCTGAGAGCCGAGGACACGGCGGTGT |
| | | ACTACTGCGCCAGAGATGTAGGAGGATACGCAGGGTACTACGGCATGGATGTATGGGGCCAGGGAACAAC |
| | | TGTCACCGTCTCCTCA |
| 1971 | BIIB-9-3654 | EVQLVESGGGLVKPGGSLRLSCAASGFTFESYSMNWVRQAPGKGLEWVSGISSASSYIYYADSVKGRFTI |
| | VH (aa) | SRDNAKNSLYLQMNSLRAEDTAVYYCARDVGGYAGYYGMDVWGQGTTVTVSS |
| 1972 | BIIB-9-3654 | GACATCCAGATGACCCAGTCTCCATCCTCCCTGTCTGCATCTGTAGGAGACAGAGTCACCATCACTTGCC |
| | VL (na) | AGGCGAGTCAGGACATTGCCAACTATTTAAATTGGTATCAGCAGAAACCAGGGAAAGCCCCTAAGCTCCT |
| | | GATCTACGATGCATCCAATTTGGAAACAGGGGTCCCATCAAGGTTCAGTGGAAGTGGATCTGGGACAGAT |
| | | TTTACTTTCACCATCAGCAGCCTGCAGCCTGAAGATATTGCAACATATTACTGTCAGCAGTACGCCAACT |
| | | TCCCTTACACTTTTGGCGGAGGGACCAAGGTTGAGATCAAA |
| 1973 | BIIB-9-3654 | DIQMTQSPSSLSASVGDRVTITCQASQDIANYLNWYQQKPGKAPKLLIYDASNLETGVPSRFSGSGSGTD |
| | VL (aa) | FTFTISSLQPEDIATYYCQQYANFPYTFGGGTKVEIK |

Parent Antibody: BIIB-9-1336

| | | |
|---|---|---|
| 1974 | BIIB-9-3753 | GAGGTGCAGCTGGTGGAGTCTGGGGGAGGCCTGGTCAAGCCTGGGGGGTCCCTGAGACTCTCCTGTGCAG |
| | VH (na) | CCTCTGGATTCACCTTCGGGAGCTATGATATGAACTGGGTCCGCCAGGCTCCAGGGAAGGGGCTGGAGTG |
| | | GGTCTCATCCATTAGTGACAGTGCAAGTTACATAGCCTACGCAGACTCAGTGAAGGGCCGATTCACCATC |
| | | TCCAGAGACAACGCCAAGAACTCACTGTATCTGCAAATGAACAGCCTGAGAGCCGAGGACACGGCGGTGT |
| | | ACTACTGCGCCAGAGATGTTTCGGGATACGCAGGGTACTACGGCATGGATGTATGGGGGCAAGGGACCAC |
| | | GGTCACCGTCTCCTCA |
| 1975 | BIIB-9-3753 | EVQLVESGGGLVKPGGSLRLSCAASGFTFGSYDMNWVRQAPGKGLEWVSSISDSASYIAYADSVKGRFTI |
| 1976 | BIIB-9-3753 | GACATCCAGATGACCCAGTCTCCATCCTCCCTGTCTGCATCTGTAGGAGACAGAGTCACCATCACTTGCC |
| | VL (na) | AGGCGAGTCAGGACATTGCCAACTATTTAAATTGGTATCAGCAGAAACCAGGGAAAGCCCCTAAGCTCCT |
| | | GATCTACGATGCATCCAATTTGGAAACAGGGGTCCCATCAAGGTTCAGTGGAAGTGGATCTGGGACAGAT |
| | | TTTACTTTCACCATCAGCAGCCTGCAGCCTGAAGATATTGCAACATATTACTGTCAGCAGTACGCCAACT |
| | | TCCCTTACACTTTTGGCGGAGGGACCAAGGTTGAGATCAAA |
| 1977 | BIIB-9-3753 | DIQMTQSPSSLSASVGDRVTITCQASQDIANYLNWYQQKPGKAPKLLIYDASNLETGVPSRFSGSGSGTD |
| | VL (aa) | FTFTISSLQPEDIATYYCQQYANFPYTFGGGTKVEIK |
| 1978 | BIIB-9-3754 | GAGGTGCAGCTGGTGGAGTCTGGGGGAGGCCTGGTCAAGCCTGGGGGGTCCCTGAGACTCTCCTGTGCAG |
| | VH (na) | CCTCTGGATTCACCTTCGGGAGCTATGATATGAACTGGGTCCGCCAGGCTCCAGGGAAGGGGCTGGAGTG |
| | | GGTCTCATCCATTAGTAGTGGTGAGAGTTACATATACTACGCAGAGTCAGTGAAGGGCCGATTCACCATC |
| | | TCCAGAGACAACGCCAAGAACTCACTGTATCTGCAAATGAACAGCCTGAGAGCCGAGGACACGGCGGTGT |
| | | ACTACTGCGCCAGAGATGTAGGAGGATACGCAGGGTTTTATGGCATGGATGTATGGGGGCAAGGGACCAC |
| | | GGTCACCGTCTCCTCA |
| 1979 | BIIB-9-3754 | EVQLVESGGGLVKPGGSLRLSCAASGFTFGSYDMNWVRQAPGKGLEWVSSISSGESYIYYAESVKGRFTI |
| | VH (aa) | SRDNAKNSLYLQMNSLRAEDTAVYYCARDVGGYAGFYGMDVWGQGTTVTVSS |
| 1980 | BIIB-9-3754 | GACATCCAGATGACCCAGTCTCCATCCTCCCTGTCTGCATCTGTAGGAGACAGAGTCACCATCACTTGCC |
| | VL (na) | AGGCGAGTCAGGACATTGCCAACTATTTAAATTGGTATCAGCAGAAACCAGGGAAAGCCCCTAAGCTCCT |
| | | GATCTACGATGCATCCAATTTGGAAACAGGGGTCCCATCAAGGTTCAGTGGAAGTGGATCTGGGACAGAT |
| | | TTTACTTTCACCATCAGCAGCCTGCAGCCTGAAGATATTGCAACATATTACTGTCAGCAGTACGCCAACT |
| | | TCCCTTACACTTTTGGCGGAGGGACCAAGGTTGAGATCAAA |
| 1981 | BIIB-9-3754 | DIQMTQSPSSLSASVGDRVTITCQASQDIANYLNWYQQKPGKAPKLLIYDASNLETGVPSRFSGSGSGTD |
| | VL (aa) | FTFTISSLQPEDIATYYCQQYANFPYTFGGGTKVEIK |
| 1982 | BIIB-9-3756 | GAGGTGCAGCTGGTGGAGTCTGGGGGAGGCCTGGTCAAGCCTGGGGGGTCCCTGAGACTCTCCTGTGCAG |
| | VH (na) | CCTCTGGATTCACCTTCGGGAGCTATGATATGAACTGGGTCCGCCAGGCTCCAGGGAAGGGGCTGGAGTG |
| | | GGTCTCATCCATTAGTAGTGGTGAGAGTTACATATACTACGCAGAGTCAGTGAAGGGCCGATTCACCATC |
| | | TCCAGAGACAACGCCAAGAACTCACTGTATCTGCAAATGAACAGCCTGAGAGCCGAGGACACGGCGGTGT |
| | | ACTACTGCGTCAGAGATGTAGGAGGATACGCAGGGTACTACGGCATGGATGTATGGGGGCAAGGGACCAC |
| | | GGTCACCGTCTCCTCA |
| 1983 | BIIB-9-3756 | EVQLVESGGGLVKPGGSLRLSCAASGFTFGSYDMNWVRQAPGKGLEWVSSISSGESYIYYAESVKGRFTI |
| | VH (aa) | SRDNAKNSLYLQMNSLRAEDTAVYYCVRDVGGYAGYYGMDVWGQGTTVTVSS |
| 1984 | BIIB-9-3756 | GACATCCAGATGACCCAGTCTCCATCCTCCCTGTCTGCATCTGTAGGAGACAGAGTCACCATCACTTGCC |
| | VL (na) | AGGCGAGTCAGGACATTGCCAACTATTTAAATTGGTATCAGCAGAAACCAGGGAAAGCCCCTAAGCTCCT |
| | | GATCTACGATGCATCCAATTTGGAAACAGGGGTCCCATCAAGGTTCAGTGGAAGTGGATCTGGGACAGAT |
| | | TTTACTTTCACCATCAGCAGCCTGCAGCCTGAAGATATTGCAACATATTACTGTCAGCAGTACGCCAACT |
| | | TCCCTTACACTTTTGGCGGAGGGACCAAGGTTGAGATCAAA |
| 1985 | BIIB-9-3756 | DIQMTQSPSSLSASVGDRVTITCQASQDIANYLNWYQQKPGKAPKLLIYDASNLETGVPSRFSGSGSGTD |
| | VL (aa) | FTFTISSLQPEDIATYYCQQYANFPYTFGGGTKVEIK |
| 1986 | BIIB-9-3764 | GAGGTGCAGCTGGTGGAGTCTGGGGGAGGCCTGGTCAAGCCTGGGGGGTCCCTGAGACTCTCCTGTGCAG |
| | VH (na) | CCTCTGGATTCACCTTCGGGAGCTATGATATGAACTGGGTCCGCCAGGCTCCAGGGAAGGGGCTGGAGTG |
| | | GGTCTCATCCATTAGTAGTGGTGAGAGTTACATATACTACGCAGAGTCAGTGAAGGGCCGATTCACCATC |
| | | TCCAGAGACAACGCCAAGAACTCACTGTATCTGCAAATGAACAGCCTGAGAGCCGAGGACACGGCGGTGT |
| | | ACTACTGCGCCAGAGATGTAGGAGGATACGCAGGGTACTACGGCATGGATGTATGGGGCCAGGGAACAAC |
| | | TGTCACCGTCTCCTCA |
| 1987 | BIIB-9-3764 | EVQLVESGGGLVKPGGSLRLSCAASGFTFGSYDMNWVRQAPGKGLEWVSSISSGESYIYYAESVKGRFTI |
| | VH (aa) | SRDNAKNSLYLQMNSLRAEDTAVYYCARDVGGYAGYYGMDVWGQGTTVTVSS |
| 1988 | BIIB-9-3764 | GACATCCAGATGACCCAGTCTCCATCCTCCCTGTCTGCATCTGTAGGAGACAGAGTCACCATCACTTGCG |
| | VL (na) | GAGCGAATCAGTACATTAGCGACTATTTAAATTGGTATCAGCAGAAACCAGGGAAAGCCCCTAAGCTCCT |
| | | GATCTACGATGCAGCCAATTTGCACACAGGGGTCCCATCAAGGTTCAGTGGAAGTGGATCTGGGACAGAT |
| | | TTTACTTTCACCATCAGCAGCCTGCAGCCTGAAGATATTGCAACATATTACTGTCAGCAGTACGCCAGGT |
| | | TCCCTTACACTTTCGGCGGAGGGACCAAGGTTGAGATCAAA |
| 1989 | BIIB-9-3764 | DIQMTQSPSSLSASVGDRVTITCGANQYISDYLNWYQQKPGKAPKLLIYDAANLHTGVPSRFSGSGSGTD |
| | VL (aa) | FTFTISSLQPEDIATYYCQQYARFPYTFGGGTKVEIK |
| 1990 | BIIB-9-3766 | GAGGTGCAGCTGGTGGAGTCTGGGGGAGGCCTGGTCAAGCCTGGGGGGTCCCTGAGACTCTCCTGTGCAG |
| | VH (na) | CCTCTGGATTCACCTTCGGGAGCTATGATATGAACTGGGTCCGCCAGGCTCCAGGGAAGGGGCTGGAGTG |
| | | GGTCTCATCCATTAGTAGTGGTGAGAGTTACATATACTACGCAGAGTCAGTGAAGGGCCGATTCACCATC |
| | | TCCAGAGACAACGCCAAGAACTCACTGTATCTGCAAATGAACAGCCTGAGAGCCGAGGACACGGCGGTGT |
| | | ACTACTGCGCCAGAGATGTAGGAGGATACGCAGGGTACTACGGCATGGATGTATGGGGCCAGGGAACAAC |
| | | TGTCACCGTCTCCTCA |
| 1991 | BIIB-9-3766 | EVQLVESGGGLVKPGGSLRLSCAASGFTFGSYDMNWVRQAPGKGLEWVSSISSGESYIYYAESVKGRFTI |
| | VH (aa) | SRDNAKNSLYLQMNSLRAEDTAVYYCARDVGGYAGYYGMDVWGQGTTVTVSS |
| 1992 | BIIB-9-3766 | GACATCCAGATGACCCAGTCTCCATCCTCCCTGTCTGCATCTGTAGGAGACAGAGTCACCATCACTTGCG |
| | VL (na) | AAGCGAGTGAAGACATTAGCAACTATTTAAATTGGTATCAGCAGAAACCAGGGAAAGCCCCTAAGCTCCT |
| | | GATCTACGATGCATCCAATTTGCAATACGGGGTCCCATCAAGGTTCAGTGGAAGTGGATCTGGGACAGAT |
| | | TTTACTTTCACCATCAGCAGCCTGCAGCCTGAAGATATTGCAACATATTACTGTAGTCAGTACGCCAACT |
| | | TCCCTTACACTTTCGGCGGAGGGACCAAGGTTGAGATCAAA |
| 1993 | BIIB-9-3766 | DIQMTQSPSSLSASVGDRVTITCEASEDISNYLNWYQQKPGKAPKLLIYDASNLQYGVPSRFSGSGSGTD |
| | VL (aa) | FTFTISSLQPEDIATYYCSQYANFPYTFGGGTKVEIK |

Parent Antibody: BIIB-9-619

| | | |
|---|---|---|
| 1994 | BIIB-9-3707 | CAGGTGCAGCTGGTGCAGTCTGGGGCTGAGGTGAGGAAGCCTGGGGCCTCAGTGAAGGTTTCCTGCAAGG |
| | VH (na) | CATCTGGATACACCTTCACCAGCTACTATATGCACTGGGTGCGACAGGCCCCTGGACAAGGGCTTGAGTG |
| | | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGACGGACCAAGAGAGTCGGACTACTACATGGACGTATGGGGCAAAGGGACCACGGT |
| | | CACCGTCTCCTCA |
| 1995 | BIIB-9-3707 | QVQLVQSGAEVRKPGASVKVSCKASGYTFTSYYMHWVRQAPGQGLEWMGIINPSGGSTSYAQKFQGRVTM |
| | VH faa) | TRDTSTSTVYMELSSLRSEDTAVYYCARDGPRESDYYMDVWGKGTTVTVSS |
| 1996 | BIIB-9-3707 | GAAATTGTGTTGACACAGTCTCCAGCCACCCTGTCTTTGTCTCCAGGGGAAAGAGCCACCCTCTCCTGCA |
| | VL (na) | GGGCCAGTCAGAGTGTTAGCAGCTACTTAGCCTGGTACCAACAGAAACCTGGCCAGGCTCCCAGGCTCCT |
| | | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGAGAGACAACT |
| | | GGCCTTTCACTTTTGGCGGAGGGACCAAGGTTGAGATCAAA |
| 1997 | BIIB-9-3707 | EIVLTQSPATLSLSPGERATLSCRASQSVSSYLAWYQQKPGQAPRLLIYDASNRATGIPARFSGSGSGTD |
| | VL (aa) | FTLTISSLEPEDFAVYYCQQRDNWPFTFGGGTKVEIK |
| 1998 | BIIB-9-3709 | CAGGTGCAGCTGGTGCAGTCTGGGGCTGAGGTGAGGAAGCCTGGGGCCTCAGTGAAGGTTTCCTGCAAGG |
| | VH (na) | CATCTGGATACACCTTCACCAGCTACTATATGCACTGGGTGCGACAGGCCCCTGGACAAGGGCTTGAGTG |
| | | GGTGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGACGGACCAAGAGATGTGGACTACTACATGGACGTATGGGGCAAAGGGACCACGGT |
| | | CACCGTCTCCTCA |
| 1999 | BIIB-9-3709 | QVQLVQSGAEVRKPGASVKVSCKASGYTFTSYYMHWVRQAPGQGLEWVGIINPSGGSTSYAQKFQGRVTM |
| | VH (aa) | TRDTSTSTVYMELSSLRSEDTAVYYCARDGPRDVDYYMDVWGKGTTVTVSS |
| 2000 | BIIB-9-3709 | GAAATTGTGTTGACACAGTCTCCAGCCACCCTGTCTTTGTCTCCAGGGGAAAGAGCCACCCTCTCCTGCA |
| | VL (na) | GGGCCAGTCAGAGTGTTAGCAGCTACTTAGCCTGGTACCAACAGAAACCTGGCCAGGCTCCCAGGCTCCT |
| | | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGAGAGACAACT |
| | | GGCCTTTCACTTTTGGCGGAGGGACCAAGGTTGAGATCAAA |
| 2001 | BIIB-9-3709 | EIVLTQSPATLSLSPGERATLSCRASQSVSSYLAWYQQKPGQAPRLLIYDASNRATGIPARFSGSGSGTD |
| | VL (aa) | FTLTISSLEPEDFAVYYCQQRDNWPFTFGGGTKVEIK |
| 2002 | BIIB-9-3720 | CAGGTGCAGCTGGTGCAGTCTGGGGCTGAGGTGAGGAAGCCTGGGGCCTCAGTGAAGGTTTCCTGCAAGG |
| | VH (na) | CATCTGGATACACCTTCACCAGCTACTATATGCACTGGGTGCGACAGGCCCCTGGACAAGGGCTTGAGTG |
| | | GATGGGAATAATCAACCCTAGTGGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGACGGACCACAGCTTAGTGACTACTACATGGACGTATGGGGCAAAGGGACCACGGT |
| | | CACCGTCTCCTCA |
| 2003 | BIIB-9-3720 | QVQLVQSGAEVRKPGASVKVSCKASGYTFTSYYMHWVRQAPGQGLEWMGIINPSGGSTSYAQKFQGRVTM |
| | VH (aa) | TRDTSTSTVYMELSSLRSEDTAVYYCARDGPQLSDYYMDVWGKGTTVTVSS |
| 2004 | BIIB-9-3720 | GAAATTGTGTTGACACAGTCTCCAGCCACCCTGTCTTTGTCTCCAGGGGAAAGAGCCACCCTCTCCTGCA |
| | VL (na) | GGGCCAGTCAGAGTGTTAGCAGCTACTTAGCCTGGTACCAACAGAAACCTGGCCAGGCTCCCAGGCTCCT |
| | | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGAGAGACAACT |
| | | GGCCTTTCACTTTTGGCGGAGGGACCAAGGTTGAGATCAAA |
| 2005 | BIIB-9-3720 | EIVLTQSPATLSLSPGERATLSCRASQSVSSYLAWYQQKPGQAPRLLIYDASNRATGIPARFSGSGSGTD |
| | VL (aa) | FTLTISSLEPEDFAVYYCQQRDNWPFTFGGGTKVEIK |
| 2006 | BIIB-9-3727 | CAGGTGCAGCTGGTGCAGTCTGGGGCTGAGGTGAAGAAGCCTGGGGCCTCAGTGAAGGTTTCCTGCAAGG |
| | VH (na) | CATCTGGATACACCTTCCATCATTACTATATGCACTGGGTGCGACAGGCCCCTGGACAAGGGCTTGAGTG |
| | | GATGGGAATAATCAACCCTAGTGGTGGTCGGACAGAGTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGACGGACCAAGAGTCAGTGACTACTACATGGACGTATGGGGCAAGGGTACAACTGT |
| | | CACCGTCTCCTCA |
| 2007 | BIIB-9-3727 | QVQLVQSGAEVKKPGASVKVSCKASGYTFHHYYMHWVRQAPGQGLEWMGIINPSGGRTEYAQKFQGRVTM |
| | VH (aa) | TRDTSTSTVYMELSSLRSEDTAVYYCARDGPRVSDYYMDVWGKGTTVTVSS |
| 2008 | BIIB-9-3727 | GAAATTGTGTTGACACAGTCTCCAGCCACCCTGTCTTTGTCTCCAGGGGAAAGAGCCACCCTCTCCTGCA |
| | VL (na) | GGGCCAGTCAGAGTGTTAGCAGCTACTTAGCCTGGTACCAACAGAAACCTGGCCAGGCTCCCAGGCTCCT |
| | | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGAGAGACAACT |
| | | GGCCTTTCACTTTTGGCGGAGGGACCAAGGTTGAGATCAAA |
| 2009 | BIIB-9-3727 | EIVLTQSPATLSLSPGERATLSCRASQSVSSYLAWYQQKPGQAPRLLIYDASNRATGIPARFSGSGSGTD |
| | VL (aa) | FTLTISSLEPEDFAVYYCQQRDNWPFTFGGGTKVEIK |
| 2010 | BIIB-9-3745 | CAGGTGCAGCTGGTGCAGTCTGGGGCTGAGGTGAAGAAGCCTGGGGCCTCAGTGAAGGTTTCCTGCAAGG |
| | VH (na) | CATCTGGATACACCTTCACCGGTTACCCTATGCACTGGGTGCGACAGGCCCCTGGACAAGGGCTTGAGTG |
| | | GATGGGATCGATCAACCCTAGTCGTGGTAGCACAAGCTACGCACAGAAGTTCCAGGGCAGAGTCACCATG |
| | | ACCAGGGACACGTCCACGAGCACAGTCTACATGGAGCTGAGCAGCCTGAGATCTGAGGACACGGCGGTGT |
| | | ACTACTGCGCCAGAGACGGACCAAGAGTCAGTGACTACTACATGGACGTATGGGGCAAGGGTACAACTGT |
| | | CACCGTCTCCTCA |
| 2011 | BIIB-9-3745 | QVQLVQSGAEVKKPGASVKVSCKASGYTFTGYPMHWVRQAPGQGLEWMGSINPSRGSTSYAQKFQGRVTM |
| | VH (aa) | TRDTSTSTVYMELSSLRSEDTAVYYCARDGPRVSDYYMDVWGKGTTVTVSS |
| 2012 | BIIB-9-3745 | GAAATTGTGTTGACACAGTCTCCAGCCACCCTGTCTTTGTCTCCAGGGGAAAGAGCCACCCTCTCCTGCA |
| | VL (na) | GGGCCAGTCAGAGTGTTAGCAGCTACTTAGCCTGGTACCAACAGAAACCTGGCCAGGCTCCCAGGCTCCT |
| | | CATCTATGATGCATCCAACAGGGCCACTGGCATCCCAGCCAGGTTCAGTGGCAGTGGGTCTGGGACAGAC |
| | | TTCACTCTCACCATCAGCAGCCTAGAGCCTGAAGATTTTGCAGTTTATTACTGTCAGCAGAGAGACAACT |
| | | GGCCTTTCACTTTTGGCGGAGGGACCAAGGTTGAGATCAAA |
| 2013 | BIIB-9-3745 | EIVLTQSPATLSLSPGERATLSCRASQSVSSYLAWYQQKPGQAPRLLIYDASNRATGIPARFSGSGSGTD |
| | VL (aa) | FTLTISSLEPEDFAVYYCQQRDNWPFTFGGGTKVEIK |

Parent Antibody: BIIB-9-578

| | | |
|---|---|---|
| 2014 | BIIB-9-3780 | CAGCTGCAGCTGCAGGAGTCGGGCCCAGGACTGGTGAAGCCTTCGGAGACCCTGTCCCTCACCTGCACTG |
| | VH (na) | TCTCTGGTGGCTCCATCAGCAGTAGTAGTTACTACTGGGGCTGGATCCGCCAGCCCCCAGGGAAGGGGCT |
| | | GGAGTGGATTGGGAGTATCTCCTATAGTGGGAGCACCTACTACAACCCGTCCCTCAAGAGTCGAGTCACC |
| | | ATATCCGTAGACACGTCCAAGAACCAGTTCTCCCTGAAGCTGAGTTCTGTGACCGCCGCAGACACGGCGG |
| | | TGTACTACTGCGCTAGAGATAAGTACCAAGACTATAGTGTTGACATATGGGGCCAAGGGACAATGGTCAC |
| | | CGTCTCCTCA |
| 2015 | BIIB-9-3780 | QLQLQESGPGLVKPSETLSLTCTVSGGSISSSSYYWGWIRQPPGKGLEWIGSISYSGSTYYNPSLKSRVT |
| | VH (aa) | ISVDTSKNQFSLKLSSVTAADTAVYYCARDKYQDYSVDIWGQGTMVTVSS |
| 2016 | BIIB-9-3780 | GACATCCAGATGACCCAGTCTCCATCTTCCGTGTCTGCATCTGTAGGAGACAGAGTCACCATCACTTGTC |
| | VL (na) | GGGCGAGTCAGGGTATTGACAGCTGGTTAGCCTGGTATCAGCAGAAACCAGGGAAAGCCCCTAAGCTCCT |
| | | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGCAAATTTCC |
| | | TCCCTTTCACTTTTGGCGGAGGGACCAAGGTTGAGATCAAA |
| 2017 | BIIB-9-3780 | DIQMTQSPSSVSASVGDRVTITCRASQGIDSWLAWYQQKPGKAPKLLIYAASSLQSGVPSRFSGSGSGTD |
| | VL (aa) | FTLTISSLQPEDFATYYCQQANFLPFTFGGGTKVEIK |
| 2018 | BIIB-9-3675 | CAGCTGCAGCTGCAGGAGTCGGGCCCAGGACTGGTGAAGCCTTCGGAGACCCTGTCCCTCACCTGCACTG |
| | VH (na) | TCTCTGGTGGCTCCATCAGCAGTACGAGTTACTACTGGGTGTGGATCCGCCAGCCCCCAGGGAAGGGGCT |
| | | GGAGTGGATTGGGAGTATCACTGCGAGTGGGAGCACCTACTACAACCCGTCCCTCAAGAGTCGAGTCACC |
| | | ATATCCGTAGACACGTCCAAGAACCAGTTCTCCCTGAAGCTGAGTTCTGTGACCGCCGCAGACACGGCGG |
| | | TGTACTACTGCGCCAGAGATAAGTACCAAGACTATTCATTCGACATATGGGGTCAGGGTACAATGGTCAC |
| | | CGTCTCCTCA |
| 2019 | BIIB-9-3675 | QLQLQESGPGLVKPSETLSLTCTVSGGSISSTSYYWVWIRQPPGKGLEWIGSITASGSTYYNPSLKSRVT |
| | VH (aa) | ISVDTSKNQFSLKLSSVTAADTAVYYCARDKYQDYSFDIWGQGTMVTVSS |
| 2020 | BIIB-9-3675 | GACATCCAGATGACCCAGTCTCCATCTTCCGTGTCTGCATCTGTAGGAGACAGAGTCACCATCACTTGTC |
| | VL (na) | GGGCGAGTCAGGGTATTGACAGCTGGTTAGCCTGGTATCAGCAGAAACCAGGGAAAGCCCCTAAGCTCCT |
| | | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGCAAATTTCC |
| | | TCCCTTTCACTTTTGGCGGAGGGACCAAGGTTGAGATCAAA |
| 2021 | BIIB-9-3675 | DIQMTQSPSSVSASVGDRVTITCRASQGIDSWLAWYQQKPGKAPKLLIYAASSLQSGVPSRFSGSGSGTD |
| | VL (aa) | FTLTISSLQPEDFATYYCQQANFLPFTFGGGTKVEIK |
| 2022 | BIIB-9-3681 | CAGCTGCAGCTGCAGGAGTCGGGCCCAGGACTGGTGAAGCCTTCGGAGACCCTGTCCCTCACCTGCACTG |
| | VH (na) | TCTCTGGTGGCTCCATCAGCAGTGGGAGTTACTACTGGAATTGGATCCGCCAGCCCCCAGGGAAGGGGCT |
| | | GGAGTGGATTGGGAGTATCCAGCCTAGTGGGAGCACCTACTACAACCCGTCCCTCAAGAGTCGAGTCACC |
| | | ATATCCGTAGACACGTCCAAGAACCAGTTCTCCCTGAAGCTGAGTTCTGTGACCGCCGCAGACACGGCGG |
| | | TGTACTACTGCGCTAGAGATAAGTACCAAGACTATTCATTCGACATATGGGGTCAGGGTACAATGGTCAC |
| | | CGTCTCCTCA |
| 2023 | BIIB-9-3681 | QLQLQESGPGLVKPSETLSLTCTVSGGSISSGSYYWNWIRQPPGKGLEWIGSIQPSGSTYYNPSLKSRVT |
| | VH (aa) | ISVDTSKNQFSLKLSSVTAADTAVYYCARDKYQDYSFDIWGQGTMVTVSS |
| 2024 | BIIB-9-3681 | GACATCCAGATGACCCAGTCTCCATCTTCCGTGTCTGCATCTGTAGGAGACAGAGTCACCATCACTTGTC |
| | VL (na) | GGGCGAGTCAGGGTATTGACAGCTGGTTAGCCTGGTATCAGCAGAAACCAGGGAAAGCCCCTAAGCTCCT |
| | | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGCAAATTTCC |
| | | TCCCTTTCACTTTTGGCGGAGGGACCAAGGTTGAGATCAAA |
| 2025 | BIIB-9-3681 | DIQMTQSPSSVSASVGDRVTITCRASQGIDSWLAWYQQKPGKAPKLLIYAASSLQSGVPSRFSGSGSGTD |
| | VL (aa) | FTLTISSLQPEDFATYYCQQANFLPFTFGGGTKVEIK |
| 2026 | BIIB-9-3684 | CAGCTGCAGCTGCAGGAGTCGGGCCCAGGACTGGTGAAGCCTTCGGAGACCCTGTCCCTCACCTGCACTG |
| | VH (na) | TCTCTGGTGGCTCCATCAGCAGTGTTAGTTACTACTGGAATTGGATCCGCCAGCCCCCAGGGAAGGGGCT |
| | | GGAGTGGATTGGGAGTATCACTTATAGTGGGAGCACCCAGTACAACCCGTCCCTCAAGAGTCGAGTCACC |
| | | ATATCCGTAGACACGTCCAAGAACCAGTTCTCCCTGAAGCTGAGTTCTGTGACCGCCGCAGACACGGCGG |
| | | TGTACTACTGCGCTAGAGATAAGTACCAAGACTATTCATTCGACATATGGGGTCAGGGTACAATGGTCAC |
| | | CGTCTCCTCA |
| 2027 | BIIB-9-3684 | QLQLQESGPGLVKPSETLSLTCTVSGGSISSVSYYWNWIRQPPGKGLEWIGSITYSGSTQYNPSLKSRVT |
| | VH (aa) | ISVDTSKNQFSLKLSSVTAADTAVYYCARDKYQDYSFDIWGQGTMVTVSS |
| 2028 | BIIB-9-3684 | GACATCCAGATGACCCAGTCTCCATCTTCCGTGTCTGCATCTGTAGGAGACAGAGTCACCATCACTTGTC |
| | VL (na) | GGGCGAGTCAGGGTATTGACAGCTGGTTAGCCTGGTATCAGCAGAAACCAGGGAAAGCCCCTAAGCTCCT |
| | | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGCAAATTTCC |
| | | TCCCTTTCACTTTTGGCGGAGGGACCAAGGTTGAGATCAAA |
| 2029 | BIIB-9-3684 | DIQMTQSPSSVSASVGDRVTITCRASQGIDSWLAWYQQKPGKAPKLLIYAASSLQSGVPSRFSGSGSGTD |
| | VL (aa) | FTLTISSLQPEDFATYYCQQANFLPFTFGGGTKVEIK |
| 2030 | BIIB-9-3698 | CAGCTGCAGCTGCAGGAGTCGGGCCCAGGACTGGTGAAGCCTTCGGAGACCCTGTCCCTCACCTGCACTG |
| | VH (na) | TCTCTGGTGGCTCCATCGCTAGTAGTAGTTACTACTGGTCGTGGATCCGCCAGCCCCCAGGGAAGGGGCT |
| | | GGAGTGGATTGGGAGTATCCGGGGTAGTGGGAGCACCTACTACAACCCGTCCCTCAAGAGTCGAGTCACC |
| | | ATATCCGTAGACACGTCCAAGAACCAGTTCTCCCTGAAGCTGAGTTCTGTGACCGCCGCAGACACGGCGG |
| | | TGTACTACTGCGCTAGAGATAAGTACCAAGACTATTCATTCGACATATGGGGTCAGGGTACAATGGTCAC |
| | | CGTCTCCTCA |
| 2031 | BIIB-9-3698 | QLQLQESGPGLVKPSETLSLTCTVSGGSIASSSYYWSWIRQPPGKGLEWIGSIRGSGSTYYNPSLKSRVT |
| | VH (aa) | ISVDTSKNQFSLKLSSVTAADTAVYYCARDKYQDYSFDIWGQGTMVTVSS |
| 2032 | BIIB-9-3698 | GACATCCAGATGACCCAGTCTCCATCTTCCGTGTCTGCATCTGTAGGAGACAGAGTCACCATCACTTGTC |
| | VL (na) | GGGCGAGTCAGGGTATTGACAGCTGGTTAGCCTGGTATCAGCAGAAACCAGGGAAAGCCCCTAAGCTCCT |
| | | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGCAAATTTCC |
| | | TCCCTTTCACTTTTGGCGGAGGGACCAAGGTTGAGATCAAA |
| 2033 | BIIB-9-3698 | DIQMTQSPSSVSASVGDRVTITCRASQGIDSWLAWYQQKPGKAPKLLIYAASSLQSGVPSRFSGSGSGTD |
| | VL (aa) | FTLTISSLQPEDFATYYCQQANFLPFTFGGGTKVEIK |
| 2034 | BIIB-9-3704 | CAGGTGCAGCTGCAGGAGTCGGGCCCAGGACTGGTGAAGCCTTCACAGACCCTGTCCCTCACCTGTACTG |
| | VH (na) | TCTCTGGTGGCTCCATCAGCAGTGGTGCGTACGCGTGGAGCTGGATCCGCCAGCACCCAGGGAAGGGCCT |
| | | GGAGTGGATTGGGTACATCTATTACCAGGGGAAGACCTACTACAACCCGTCCCTCAAGAGTCGAGTTACC |
| | | ATATCAGTAGACACGTCTAAGAACCAGTTCTCCCTGAAGCTGAGTTCTGTGACCGCCGCAGACACGGCGG |
| | | TGTACTACTGCGCTAGAGATAAGTACCAAGACTATTCATTCGACATATGGGGTCAGGGTACAATGGTCAC |
| | | CGTCTCCTCA |
| 2035 | BIIB-9-3704 | QVQLQESGPGLVKPSQTLSLTCTVSGGSISSGAYAWSWIRQHPGKGLEWIGYIYYQGKTYYNPSLKSRVT |
| | VH (aa) | ISVDTSKNQFSLKLSSVTAADTAVYYCARDKYQDYSFDIWGQGTMVTVSS |
| 2036 | BIIB-9-3704 | GACATCCAGATGACCCAGTCTCCATCTTCCGTGTCTGCATCTGTAGGAGACAGAGTCACCATCACTTGTC |
| | VL (na) | GGGCGAGTCAGGGTATTGACAGCTGGTTAGCCTGGTATCAGCAGAAACCAGGGAAAGCCCCTAAGCTCCT |
| | | GATCTATGCTGCATCCAGTTTGCAAAGTGGGGTCCCATCAAGGTTCAGCGGCAGTGGATCTGGGACAGAT |
| | | TTCACTCTCACCATCAGCAGCCTGCAGCCTGAAGATTTTGCAACTTATTACTGTCAGCAGGCAAATTTCC |
| | | TCCCTTTCACTTTTGGCGGAGGGACCAAGGTTGAGATCAAA |
| 2037 | BIIB-9-3704 | DIQMTQSPSSVSASVGDRVTITCRASQGIDSWLAWYQQKPGKAPKLLIYAASSLQSGVPSRFSGSGSGTD |
| | VL (aa) | FTLTISSLQPEDFATYYCQQANFLPFTFGGGTKVEIK |

(aa) = amino acid sequence; (nt) = nucleotide sequence

TABLE 7
CDRs of Example 14 daughter antibodies

| | | | | | | |
|---|---|---|---|---|---|---|
| Antibody | VH-CDR1 | VH-CDR2 | VH-CDR3 | VL-CDR1 | VL-CDR2 | VL-CDR3 |

Parent antibody: BIIB-9-484

| | | | | | | |
|---|---|---|---|---|---|---|
| BIIB-9-3595 | 2038 | 2064 | 2090 | 2116 | 2142 | 2168 |
| BIIB-9-3601 | 2039 | 2065 | 2091 | 2117 | 2143 | 2169 |
| BIIB-9-3604 | 2040 | 2066 | 2092 | 2118 | 2144 | 2170 |
| BIIB-9-3617 | 2041 | 2067 | 2093 | 2119 | 2145 | 2171 |
| BIIB-9-3618 | 2042 | 2068 | 2094 | 2120 | 2146 | 2172 |
| BIIB-9-3621 | 2043 | 2069 | 2095 | 2121 | 2147 | 2173 |
| BIIB-9-3647 | 2044 | 2070 | 2096 | 2122 | 2148 | 2174 |
| BIIB-9-3649 | 2045 | 2071 | 2097 | 2123 | 2149 | 2175 |
| BIIB-9-3650 | 2046 | 2072 | 2098 | 2124 | 2150 | 2176 |
| BIIB-9-3654 | 2047 | 2073 | 2099 | 2125 | 2151 | 2177 |

Parent antibody: BIIB-9-1336

| | | | | | | |
|---|---|---|---|---|---|---|
| BIIB-9-3753 | 2048 | 2074 | 2100 | 2126 | 2152 | 2178 |
| BIIB-9-3754 | 2049 | 2075 | 2101 | 2127 | 2153 | 2179 |
| BIIB-9-3756 | 2050 | 2076 | 2102 | 2128 | 2154 | 2180 |
| BIIB-9-3764 | 2051 | 2077 | 2103 | 2129 | 2155 | 2181 |
| BIIB-9-3766 | 2052 | 2078 | 2104 | 2130 | 2156 | 2182 |

Parent antibody: BIIB-9-619

| | | | | | | |
|---|---|---|---|---|---|---|
| BIIB-9-3707 | 2053 | 2079 | 2105 | 2131 | 2157 | 2183 |
| BIIB-9-3709 | 2054 | 2080 | 2106 | 2132 | 2158 | 2184 |
| BIIB-9-3720 | 2055 | 2081 | 2107 | 2133 | 2159 | 2185 |
| BIIB-9-3727 | 2056 | 2082 | 2108 | 2134 | 2160 | 2185 |
| BIIB-9-3745 | 2057 | 2083 | 2109 | 2135 | 2161 | 2187 |

Parent antibody: BIIB-9-578

| | | | | | | |
|---|---|---|---|---|---|---|
| BIIB-9-3780 | 2058 | 2084 | 2110 | 2136 | 2162 | 2188 |
| BIIB-9-3675 | 2059 | 2085 | 2111 | 2137 | 2163 | 2189 |
| BIIB-9-3681 | 2060 | 2086 | 2112 | 2138 | 2164 | 2190 |
| BIIB-9-3684 | 2061 | 2087 | 2113 | 2139 | 2164 | 2191 |
| BIIB-9-3698 | 2062 | 2088 | 2114 | 2140 | 2166 | 2192 |
| BIIB-9-3704 | 2063 | 2089 | 2115 | 2141 | 2167 | 2193 |

1. An isolated antibody, or an antigen binding portion thereof, that specifically binds to activated factor IX (FIXa) (“anti-FIXa antibody or antigen binding portion thereof”), wherein the anti-FIXa antibody or antigen binding portion thereof preferentially binds to FIXa in the presence of FIXa and factor IX zymogen (FIXz) or wherein the anti-FIXa antibody, or antigen binding portion thereof binds to FIXa with a binding affinity higher than a binding affinity of the anti-FIXa antibody or antigen binding portion thereof to FIXz.

2. The anti-FIXa antibody, or antigen binding portion thereof, of claim 1, which cross-competes with or binds to the same epitope as a reference antibody selected from the group consisting of the antibodies in FIG. 3A, FIG. 3B, and FIG. 3C.

3. The anti-FIXa antibody, or antigen binding portion thereof, of claim 3 wherein the epitope comprises chymotrypsinogen numbering amino acid residues H91, H92, N93, H101, D125, K126, E127, Y128, R165, Y177, N178, N179, S232, R233, Y234, V235, N236, W237, E240, and K241 of the sequence of the heavy chain of FIXa, or a combination thereof.

4. The anti-FIXa antibody, or antigen binding portion thereof, of claim 1 which comprises VH CDR1, VH CDR2, and VH CDR3, wherein
(i) the VH CDR1 comprises a VH CDR1 selected from the group consisting of VH CDR1s in FIG. 3A, FIG. 3B, and FIG. 3C, SEQ ID NOs: 2058 to 2063, or the VH CDR1 with one or two mutations; and/or
(ii) the VH CDR2 comprises a VH CDR2 selected from the group consisting of VH CDR2s in FIG. 3A, FIG. 3B, and FIG. 3C, SEQ ID NOs: 2084 to 2089, or the VH CDR2 with one or two mutations; and/or
(iii) the VH CDR3 comprises a VH CDR3 selected from the group consisting of VH CDR3s in FIG. 3A, FIG. 3B, and FIG. 3C, SEQ ID NOs: 2084 to 2089, or the VH CDR3 with one or two mutations; and/or wherein
(iv) the VL CDR1 comprises a VL CDR1 selected from the group consisting of VL CDR1s in FIG. 3A, FIG. 3B, and FIG. 3C, SEQ ID NOs: 2136 to 2141, or the VL CDR1 with one or two mutations; and/or
(v) the VL CDR2 comprises a VL CDR2 selected from the group consisting of VL CDR2s in FIG. 3A, FIG. 3B, and FIG. 3C, SEQ ID NOs: 2162 to 2167, or the VL CDR2 with one or two mutations; and/or
(vi) the VL CDR3 comprises a VL CDR3 selected from the group consisting of VL CDR3s in FIG. 3A, FIG. 3B, and FIG. 3C, SEQ ID NOs: 2188 to 2193, or the VL CDR3 with one or two mutations.

5. The anti-FIXa antibody, or antigen binding portion thereof of claim 1 comprising VH and VL, wherein (a1) VH and VL comprise SEQ ID NOs: 31 and 221, respectively (BIIB-9-484); (a2) VH and VL comprise SEQ ID NOs: 19 and 209, respectively (BIIB-9-440); (a3) VH and VL comprise SEQ ID NOs: 115 and 301, respectively (BIIB-9-882); (a4) VH and VL comprise SEQ ID NOs: 23 and 213, respectively (BIIB-9-460); (a5) VH and VL comprise SEQ ID NOs: 127 and 313, respectively (BIIB-9-433); (a6) VH and VL comprise SEQ ID NOs: 45 and 235, respectively (BIIB-9-619); (a7) VH and VL comprise SEQ ID NOs: 185 and 371, respectively (BIIB-9-578); (a8) VH and VL comprise SEQ ID NOs: 87 and 221, respectively (BIIB-9-1335); or, (a9) VH and VL comprise SEQ ID NOs: 89 and 221, respectively (BIIB-9-1336).

6. An isolated antibody, or an antigen binding portion thereof, that specifically binds to factor X zymogen (FXz) (“anti-FXz antibody or antigen binding portion thereof”), wherein the anti-FXz antibody or antigen binding portion thereof preferentially binds to FXz in the presence of FXz and activated factor X (FXa).

7. The anti-FXz antibody, or antigen binding portion thereof, of claim 6, which cross-competes with or binds to the same epitope as a reference antibody selected from the group consisting of the antibodies in FIG. 12A and FIG. 12B.

8. The anti-FXz antibody, or antigen binding portion thereof, of claim 6, comprising VH and VL, wherein (b1) VH and VL comprise SEQ ID NOs: 423 and 611, respectively (BIIB-12-915); (b2) VH and VL comprise SEQ ID NOs: 427 and 615, respectively (BIIB-12-917); or, (b3) VH and VL comprise SEQ ID NOs: 455 and 643, respectively (BIIB-12-932).

9. An isolated antibody, or an antigen binding portion thereof, that specifically binds to activated factor X (FXa) (“anti-FXa antibody or antigen binding portion thereof”), wherein the anti-FXa antibody or antigen binding portion thereof preferentially binds to FXa in the presence of FXz and FXa and/or binds to FXa with a binding affinity higher than a binding affinity of the antibody or antigen binding portion thereof to FXz.

10. The anti-FXa antibody, or antigen binding portion thereof, of claim 9, which cross-competes with a reference antibody selected from the group consisting of the antibodies in FIG. 12C or binds to the same epitope as a reference antibody selected from the group consisting of the antibodies in FIG. 12C.

11. The anti-FXa antibody, or antigen binding portion thereof, of claim 9, comprising VH and VL, wherein the VH and VL comprise SEQ ID NOs: 559 and 747, respectively (BIIB-12-925).

12. A bispecific molecule comprising the anti-FIX antibody, or antigen binding portion thereof, of claim 1.

13. A nucleic acid encoding the antibody, or antigen binding portion thereof, of claim 1.

14. A pharmaceutical composition comprising the antibody, or antigen binding portion thereof, of claim 1 and a pharmaceutically acceptable carrier.

15. (canceled)

16. A bispecific molecule comprising the anti-FIX antibody, or antigen binding portion thereof, of claim 6.

17. A bispecific molecule comprising the anti-FIX antibody, or antigen binding portion thereof, of claim 9.

18. A nucleic acid encoding the antibody, or antigen binding portion thereof, of claim 6.

19. A nucleic acid encoding the antibody, or antigen binding portion thereof, of claim 9.

20. A pharmaceutical composition comprising the antibody, or antigen binding portion thereof, of claim 6 and a pharmaceutically acceptable carrier.

21. A pharmaceutical composition comprising the antibody, or antigen binding portion thereof, of claim 9 and a pharmaceutically acceptable carrier.
